(19) United States
(12) Patent Application Publication
(10) Pub. No.: US 2024/0336608 A1
(43) Pub. Date: Oct. 10, 2024

(54) IL4I1 INHIBITORS AND METHODS OF USE

(71) Applicant: Merck Sharp & Dohme LLC, Rahway, NJ (US)

(72) Inventors: David A. Candito, Wrentham, MA (US); Anthony Donofrio, Medford, MA (US); Ronald Dale Ferguson, II, Basking Ridge, NJ (US); George Madalin Giambasu, Natick, MA (US); Anmol Gulati, Watertown, MA (US); Andrew M. Haidle, Somerville, MA (US); Brett A. Hopkins, Brownsburg, IN (US); William P. Kaplan, Moseley, VA (US); Ping Liu, Westfield, NJ (US); Michaelyn C. Lux, Brookline, MA (US); Xiao Mei Zheng, Dover, MA (US); Ryan D. Otte, Natick, MA (US); Alexander Pasternak, Jamaica Plain, MA (US); Elsie C. Yu, Brighton, MA (US)

(73) Assignee: Merck Sharp & Dohme LLC, Rahway, NJ (US)

(21) Appl. No.: 18/617,907

(22) Filed: Mar. 27, 2024

Related U.S. Application Data
(60) Provisional application No. 63/455,406, filed on Mar. 29, 2023.

(51) Int. Cl. C07D 417/14; A61K 31/422; A61K 31/433; A61K 31/437; A61K 31/4439; A61K 31/497; A61K 31/501; A61K 31/506; A61K 31/542; A61K 39/395; C07D 401/14; C07D 403/12; C07D 409/14; C07D 413/12; C07D 413/14; C07D 417/12; C07D 471/04; C07D 487/04; C07D 491/048; C07D 495/04; C07D 513/04

(52) U.S. Cl. CPC C07D 417/14; A61K 31/422; A61K 31/433; A61K 31/437; A61K 31/4439; A61K 31/497; A61K 31/501; A61K 31/506; A61K 31/542; A61K 39/3955; C07D 401/14; C07D 403/12; C07D 409/14; C07D 413/12; C07D 413/14; C07D 417/12; C07D 471/04; C07D 487/04; C07D 491/048; C07D 495/04; C07D 513/04

(57) ABSTRACT

Described herein are compounds of Formula I

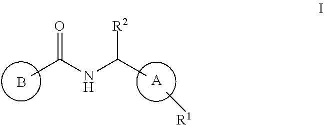

or a pharmaceutically acceptable salt thereof, wherein R¹, R², A, and B are as defined herein. The compounds of Formula I act as IL4I1 inhibitors and can be useful in preventing, treating or acting as a remedial agent for IL4I1-related diseases.

### CROSS-REFERENCE TO RELATED APPLICATIONS

This application claims priority to U.S. Provisional Application No. 63/455,406 filed Mar. 29, 2023, hereby incorporated by reference in its entirety.

### FIELD OF THE INVENTION

The invention is directed to IL4I1 inhibitor compounds. Specifically, the IL4I1 inhibitors described herein can be useful in preventing, treating or acting as a remedial agent for IL4I1-related diseases.

### BACKGROUND OF THE INVENTION

IL4I1 is a glycosylated protein that belongs to the L-amino-acid oxidase (LAAO) family of flavin adenine dinucleotide (FAD)-bound enzymes. IL4I1 is secreted from certain cells and performs oxidative deamination of phenylalanine into phenylpyruvate, liberating H₂O₂ and NH₃.

The highest production of IL4I1 is found in cells of myeloid origin (monocyte/macrophages and dendritic cells) of the human immune system, particularly after stimulation with inflammatory and T helper type 1 (Th1) stimuli. Accordingly, IL4I1 is strongly produced by dendritic cell and macrophage populations from chronic Th1 granulomas of sarcoidosis and tuberculosis, but not Th2 granulomas (schistosomiasis). Moreover, tumor-infiltrating macrophages from various histological types of tumors strongly produce IL4I1. Molinier-Frenkel V., Prévost-Blondel A. and Castellano F., The IL4I1 Enzyme: A New Player in the Immunosuppressive Tumor Microenvironment, Cells, 2019, 8, 757-765.

The presence of IL4I1-producing cells in the tumor cell microenvironment restrains the anti-tumor immune response by directly limiting the proliferation and functionality of cytotoxic T cells and Th1 cells, or indirectly by facilitating the accumulation of Treg cells. Analyses of human tumor and normal tissue biopsies have identified increased expression of both IL4I1 mRNA and protein in tumor infiltrating myeloid cells. The Cancer Genome Atlas (TCGA) indicate that, among solid tumors, endometrial carcinoma contains the highest levels of IL4I1 mRNA expression, followed by serious ovarian and triple negative breast cancers. Elevated levels have also been found in a few other cancer types like diffuse large B-cell lymphoma (DLBCL) and acute myeloid leukemia (AML). Phenylpyruvic acid, the product of phenylalanine oxidation by IL4I1, is elevated in endometrial and ovarian tumor samples relative to matched adjacent tissue from the same patients. Furthermore, accumulation of detectable phenylpyruvic acid in the tumor samples is dependent on the presence of IL4I1 itself.

Currently there are no approved specific IL4I1 inhibitors. Some molecules have been shown to inhibit the related LAAOs found in snake venom, but they are generally non-selective and have little activity. Therefore there is a need for specific inhibitors of IL4I1. More specifically, there is a need for compounds that specifically inhibit IL4I1 and can be useful for the treatment of indications where IL4I1 is most expressed and/or active, including endometrial, ovarian, and triple negative breast cancers, DLBCL and AML.

### BRIEF SUMMARY OF THE INVENTION

Described herein are compounds of Formula I:

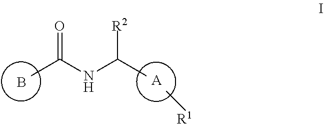

and pharmaceutically acceptable salts thereof, wherein A, B, R¹ and R² are described below.

The compounds described herein are IL4I1 inhibitors, which can be useful in the prevention, treatment or amelioration of IL4I1-related diseases.

Also described herein are methods of preventing, treating or ameliorating the symptoms of cancer comprising administering to a patient in need thereof a compound described herein, or a pharmaceutically acceptable salt thereof.

Also described herein are uses of a compound described herein, or a pharmaceutically acceptable salt thereof, to prevent, treat or ameliorate the conditions of cancer in a patient in need thereof.

Also described herein are pharmaceutical compositions comprising a compound described herein, or a pharmaceutically acceptable salt thereof, and a pharmaceutically acceptable carrier.

Also described herein are pharmaceutical compositions comprising a compound described herein and a pharmaceutically acceptable carrier.

Also described herein are methods of preventing, treating or ameliorating the symptoms of cancer comprising administering to a patient in need thereof a compound described herein, or a pharmaceutically acceptable salt thereof and another therapeutic agent.

Also described herein are uses of a compound described herein, or a pharmaceutically acceptable salt thereof, in combination with another therapeutic agent to prevent, treat or ameliorate the conditions of cancer in a patient in need thereof.

Also described herein are pharmaceutical compositions comprising a compound described herein, or a pharmaceutically acceptable salt thereof, another therapeutic agent and a pharmaceutically acceptable carrier.

Also described herein are pharmaceutical compositions comprising a compound described herein, another therapeutic agent and a pharmaceutically acceptable carrier.

### DETAILED DESCRIPTION OF THE INVENTION

Described herein are compounds of Formula I:

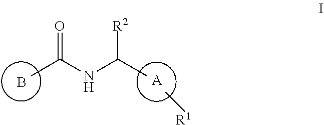

or a pharmaceutically acceptable salt thereof, wherein:
A is a five or six-membered, nitrogen-containing heteroaryl ring; B is a five-membered heteroaryl, bicyclic heterocycloalkyl or bicyclic heteroaryl, wherein the bicyclic heterocycloalkyl and bicyclic heteroaryl are unsubstituted or substituted with one to three substituents and the five-membered heteroaryl is substituted with one to three substituents, wherein the substituents are independently selected from the group consisting of aryl, haloC₁-C₆alkyl, C₁-C₆alkyl, —C₁-C₆alkylOH, alkoxy, —OH, C₁-C₆alkylaryl, C₃-C₆cycloalkyl, C₁-C₆alkylC₃-C₆cycloalkyl, heteroaryl, —C₁-C₆alkylCON(R^{a})₂, haloC₁-C₆alkoxy, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl, halogen, and —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, or —C₁-C₆alkylCOOH is unsubstituted or substituted with one to three substituents independently selected from the group consisting of C₁-C₆alkyl, halogen, —NH₂, —OH, —CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl;
R¹ is selected from C₃-C₆cycloalkyl, cycloheteroalkyl, aryl and heteroaryl, wherein the C₃-C₆cycloalkyl, cycloheteroalkyl, aryl or heteroaryl is unsubstituted or substituted with one to three substituents selected from the group consisting of alkoxy, CN, —C₁-C₆alkylOH, halogen, C₁-C₆alkyl, haloC₁-C₆alkyl, and —OH;
R² is hydrogen, C₁-C₆alkyl or haloC₁-C₆alkyl; and
R^{a} is hydrogen, C₁-C₆alkyl or haloC₁-C₆alkyl.

With regard to the compounds described herein, A is a five or six-membered, nitrogen-containing heteroaryl. In certain embodiments, A is a five-membered, nitrogen-containing ring. In certain embodiments, A is a five-membered, nitrogen-containing heteroaryl. In certain embodiments, A is a six-membered, nitrogen-containing ring. In certain embodiments, A is a six-membered, nitrogen-containing heteroaryl. In certain embodiments, A is a five-membered nitrogen containing ring, and the five-membered nitrogen containing ring is selected from the group consisting of pyrrolidine, pyrroline, pyrazolidine, pyrazoline, imidazolidine, imidazoline, pyrrole, pyrazole, imidazole, 1H-1,2,3-triazole, 4H-1,2,4-triazole, isoxazole, oxazole, 1,2,3-oxadiazole, 1,3,4-oxadiazole, furazan, 1,2,4-oxadiazole, 1,2,3,4-oxatrizole, 1,2,3,5-oxatriazole, isothiazole, thiazole, 1,2,3-thiadiazole, 1,3,4-thiadiazole, 1,2,5-thiadiazole, 1,2,4-thiadiazole, 1,2,3,4-thiatrizole and 1,2,3,5-thiatriazole.

In certain embodiments, A is a five-membered nitrogen containing heteroaryl ring, and the five-membered nitrogen containing heteroaryl ring is selected from the group consisting of

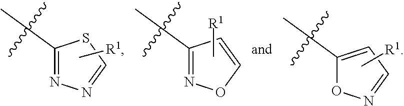

In certain embodiments, A is a six-membered nitrogen containing heteroaryl ring. In certain embodiments, A is a six-membered nitrogen containing heteroaryl ring, wherein the six-membered nitrogen containing ring is selected from the group consisting of pyridine, pyrazine, pyrimidine, and pyridazine. In certain embodiments, A is

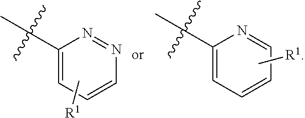

With regard to the compounds described herein, R¹ is selected from C₃-C₆cycloalkyl, cycloheteroalkyl, aryl or heteroaryl, wherein the C₃-C₆cycloalkyl, cycloheteroalkyl, aryl or heteroaryl is unsubstituted or substituted with 1 to 3 substituents selected from the group consisting of alkoxy, CN, —C₁-C₆alkylOH, halogen, C₁-C₆alkyl, haloC₁-C₆alkyl, and —OH. In certain embodiments, R¹ is selected from C₃-C₆cycloalkyl, aryl or heteroaryl, wherein the C₃-C₆cycloalkyl, aryl or heteroaryl is unsubstituted or substituted with 1 to 3 substituents selected from the group consisting of alkoxy, —CN, —C₁-C₆alkylOH, halogen, C₁-C₆alkyl, haloC₁-C₆alkyl, and —OH.

In certain embodiments, R¹ is C₃-C₆cycloalkyl. Suitable cycloalkyls include, but are not limited to, cyclopropyl, cyclobutyl, cyclopentyl, cyclohexyl, and cycloheptyl. In certain embodiments, R¹ is C₃-C₆cycloalkyl, wherein the C₃-C₆cycloalkyl is cyclopentane or cyclohexane. In certain embodiments, R¹ is C₃-C₆cycloalkyl, wherein the C₃-C₆cycloalkyl is unsubstituted. In certain embodiments, R¹ is C₃-C₆cycloalkyl, wherein the C₃-C₆cycloalkyl is substituted.

In certain embodiments, R¹ is cycloheteroalkyl. Suitable cycloheteroalkyls include, but are not limited to, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, tetrahydropyran and partially unsaturated monocyclic rings that are not aromatic, such as 2- or 4-pyridones attached through the nitrogen or N-substituted-(1H,3H)-pyrimidine-2,4-diones (N-substituted uracils). In certain embodiments, R¹ is piperidinyl. In certain embodiments, R¹ is cycloheteroalkyl, wherein the cycloheteroalkyl is unsubstituted. In certain embodiments, R¹ is cycloheteroalkyl, wherein the cycloheteroalkyl is substituted.

In certain embodiments, R¹ is aryl. Suitable aryls include, but are not limited to, phenyl and naphthyl. In certain embodiments, R¹ is aryl, wherein the aryl is phenyl. In certain embodiments, R¹ is aryl, wherein the aryl is unsubstituted. In certain embodiments, R¹ is aryl, wherein the aryl is substituted. In certain embodiments, R¹ is aryl, wherein the aryl is phenyl and the phenyl is unsubstituted.

In certain embodiments, R¹ is heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, R¹ is heteroaryl, wherein the heteroaryl is unsubstituted. In certain embodiments, R¹ is heteroaryl, wherein the heteroaryl is substituted. In certain embodiments, R¹ is heteroaryl, wherein the heteroaryl is pyridine, thiophene, thiazole, triazole and pyrazole. In certain embodiments, R¹ is heteroaryl, wherein the heteroaryl is pyridyl (pyridinyl). In certain embodiments, R¹ is heteroaryl, wherein the heteroaryls is pyrimidyl. In certain embodiments, R¹ is heteroaryl, wherein the heteroaryl is triazolyl. In certain embodiments, R¹ is heteroaryl, wherein the heteroaryl is thienyl.

In certain embodiments, R¹ is C₃-C₆cycloalkyl, cycloheteroalkyl, aryl or heteroaryl, wherein the C₃-C₆cycloalkyl, cycloheteroalkyl, aryl or heteroaryl is substituted with 1 to 3 substituents. In certain embodiments, the C₃-C₆cycloalkyl, cycloheteroalkyl, aryl or heteroaryl is substituted with 1 substituent. In certain embodiments, the C₃-C₆cycloalkyl, cycloheteroalkyl, aryl or heteroaryl is substituted with 2 substituents. In certain embodiments, the C₃-C₆cycloalkyl, cycloheteroalkyl, aryl or heteroaryl is substituted 3 substituents. In certain embodiments, the C₃-C₆cycloalkyl, cycloheteroalkyl, aryl or heteroaryl is substituted with 1 to 3 substituents elected from the group consisting of alkoxy, CN, —C₁-C₆alkylOH, halogen, C₁-C₆alkyl, haloC₁-C₆alkyl, and —OH.

In certain embodiments, R¹ is C₃-C₆cycloalkyl, cycloheteroalkyl, aryl or heteroaryl wherein the C₃-C₆cycloalkyl, cycloheteroalkyl, aryl or heteroaryl is substituted with alkoxy. Suitable alkoxys include, but are not limited to, methoxy, ethoxy, n-propoxy, isopropoxy and n-butoxy. In certain embodiments, the C₃-C₆cycloalkyl, cycloheteroalkyl, aryl or heteroaryl is substituted with —CN. In certain embodiments, the C₃-C₆cycloalkyl, cycloheteroalkyl, aryl or heteroaryl is substituted with —C₁-C₆alkylOH. In certain embodiments, the C₃-C₆cycloalkyl, cycloheteroalkyl, aryl or heteroaryl is substituted with halogen. Suitable halogens include, but are not limited to, a fluorine, a chlorine, a bromine or an iodine radical. In certain embodiments, the C₃-C₆cycloalkyl, cycloheteroalkyl, aryl or heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the C₃-C₆cycloalkyl, cycloheteroalkyl, aryl or heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₃-C₆cycloalkyl, cycloheteroalkyl, aryl or heteroaryl is substituted with —OH.

In certain embodiments, R¹ is aryl, wherein the aryl is phenyl, wherein the phenyl is substituted with one to three substituents independently selected from the group consisting of methyl, chlorine, —OH and fluorine.

In certain embodiments, R¹ is C₃-C₆cycloalkyl, wherein the C₃-C₆cycloalkyl is substituted with one to three substituents independently selected from the group consisting of methyl, chlorine, —OH and fluorine.

In certain embodiments, R¹ is heterocycloalkyl, wherein the heterocycloalkyl is substituted with one to three methyl groups.

In certain embodiments, R¹ is heteroaryl, wherein the heteroaryl is substituted with one to three substituents independently selected from the group consisting of methyl, chlorine, —OH and fluorine.

With regard to the compounds described herein, B is a five-membered heteroaryl, bicyclic heterocycloalkyl or bicyclic heteroaryl, wherein the bicyclic heterocycloalkyl and bicyclic heteroaryl are unsubstituted or substituted with one to three substituents and the five-membered heteroaryl is substituted with one to three substituents, wherein the substituents are independently selected from the group consisting of aryl, haloC₁-C₆alkyl, C₁-C₆alkyl, —C₁-C₆alkylOH, alkoxy, —OH, C₁-C₆alkylaryl, C₃-C₆cycloalkyl, C₁-C₆alkylC₃-C₆cycloalkyl, heteroaryl, —C₁-C₆alkylCON(R^{a})₂, haloC₁-C₆alkoxy, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl, halogen, and —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, or —C₁-C₆alkylCOOH is unsubstituted or substituted with 1 to 3 substituents independently selected from the group consisting of C₁-C₆alkyl, halogen, —NH₂, —OH, —CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl.

In certain embodiments, B is a five-membered heteroaryl, bicyclic heterocycloalkyl or bicyclic heteroaryl. In certain embodiments, B is a five-membered heteroaryl. Suitable five-membered heteroaryls include pyrazole, imidazole, triazole, tetrazole, furan, thiophen, oxazole, isoxazole, isothiazole, thiazole, oxadiazole and thiadiazole. In certain embodiments, B is triazole, pyrazole, isoxazole, thiophene, imidazole, thiazole, pyrrole, furan, oxadiazole, oxazole and isothiazole.

In certain embodiments, B is a bicyclic heterocycloalkyl. Suitable bicyclic heterocycloalkyls include cyclopenta[b]thiophene, dihydrothieno[3,4-b][1,4]dioxine, hydroxyhexahydrofurofuran, dihydropyrazolothiazine, dihydropyrrolopyrazole and fluoropyridine. In certain embodiments, B is a bicyclic heteroaryl. Suitable heteroaryls include, imidazole[1,2-a]pyridine, benzofuran, indole, dihydrofluoropyridine, benzoxazole and triazolopyridine.

In certain embodiments, the five-membered heteroaryl is substituted with 1 to 3 substituents. In certain embodiments, the five-membered heteroaryl is substituted with 1 substituent. In certain embodiments, the five-membered heteroaryl is substituted with 2 substituents. In certain embodiments, the five-membered heteroaryl is substituted with 3 substituents.

In certain embodiments, the bicyclic heterocycloalkyl is unsubstituted or substituted with one to three substituents. In certain embodiments, the bicyclic heteroaryl is unsubstituted or substituted with one to three substituents. In certain embodiments, the bicyclic heterocycloalkyl is unsubstituted. In certain embodiments, the bicyclic heterocycloalkyl is substituted with one to three substituents. In certain embodiments, the bicyclic heteroaryl is unsubstituted. In certain embodiments, the bicyclic heteroaryl is substituted with one to three substituents.

In certain embodiments, the bicyclic heterocycloalkyl is substituted with 1 to 3 substituents. In certain embodiments, the bicyclic heterocycloalkyl is substituted with 1 substituent. In certain embodiments, the bicyclic heterocycloalkyl is substituted with 2 substituents. In certain embodiments, the bicyclic heterocycloalkyl is substituted with 3 substituents.

In certain embodiments, the bicyclic heteroaryl is substituted with 1 to 3 substituents. In certain embodiments, the bicyclic heteroaryl is substituted with 1 substituent. In certain embodiments, the bicyclic heteroaryl is substituted with 2 substituents. In certain embodiments, the bicyclic heteroaryl is substituted with 3 substituents.

In certain embodiments, B is a bicyclic heterocycloalkyl, bicyclic heteroaryl, or five-membered heteroaryl, wherein the bicyclic heterocycloalkyl, bicyclic heteroaryl or five-membered heteroaryl are substituted with one, two or three substituents independently selected from the group consisting of aryl, haloC₁-C₆alkyl, C₁-C₆alkyl, —C₁-C₆alkylOH, alkoxy, —OH, C₁-C₆alkylaryl, C₃-C₆cycloalkyl, C₁-C₆alkylC₃-C₆cycloalkyl, heteroaryl, —C₁-C₆alkylCON(R^{a})₂, haloC₁-C₆alkoxy, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl, halogen, and —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, or —C₁-C₆alkylCOOH is unsubstituted or substituted with 1 to 3 substituents independently selected from the group consisting of C₁-C₆alkyl, halogen, —NH₂, —OH, —CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl.

In certain embodiments, B is substituted with aryl. Suitable aryls include, but are not limited to, phenyl and naphthyl. In certain embodiments, B is substituted with aryl, wherein the aryl is phenyl.

In certain embodiments, B is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, B is substituted with difluoromethyl. In certain embodiments, B is substituted with trifluoromethyl. In certain embodiments, B is substituted with fluoropropyl, trifluoroethyl, trifluoropropyl, trifluorobutyl, difluoromethyl and trifluoromethyl.

In certain embodiments, B is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, B is substituted with isopropyl, isobutyl, tertbutyl, methyl, propyl, ethylbutyl or ethyl. In certain embodiments, B is substituted with methyl. In certain embodiments, B is substituted with ethyl.

In certain embodiments, B is substituted with —C₁-C₆alkylOH. Suitable alcohols include, but are not limited to, methanol, ethanol, propanol, butanol and isopropanol. In certain embodiments, B is substituted with hydroxymethyl or hydroxyethyl.

In certain embodiments, B is substituted with alkoxy. Suitable alkoxys include, but are not limited to, methoxy, ethoxy, n-propoxy, isopropoxy and n-butoxy. In certain embodiments, B is substituted with methoxy or ethoxy.

In certain embodiments, B is substituted with —OH.

In certain embodiments, B is substituted with C₁-C₆alkylaryl. Suitable examples of —C₁-C₆alkylaryl, include any C₁-C₆alkyl as defined above wherein a hydrogen is replaced with an aryl group. In certain embodiments, B is substituted with CH₂phenyl.

In certain embodiments, B is substituted with C₃-C₆cycloalkyl. Suitable cycloalkyls include, but are not limited to, cyclopropyl, cyclobutyl, cyclopentyl, cyclohexyl, and cycloheptyl.

In certain embodiments, B is substituted with cyclobutyl, cyclohexyl, bicyclopentane or cyclopropyl.

In certain embodiments, B is substituted with C₁-C₆alkylC₃-C₆cycloalkyl. Suitable examples of C₁-C₆alkylC₃-C₆cycloalkyl, include any C₁-C₆alkyl as defined above wherein a hydrogen is replaced with a cycloalkyl group. In certain embodiments, B is substituted with CH₂cyclopentyl, CH₂cyclobutyl or CH₂cyclopentyl.

In certain embodiments, B is substituted with heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, B is substituted with pyridinyl, thiadiazolyl, pyrimidinyl or thiazolyl.

In certain embodiments, B is substituted with —C₁-C₆alkylCON(R^{a})₂, wherein R^{a} is selected from the group consisting of hydrogen, C₁-C₆alkyl and haloC₁-C₆alkyl. In certain embodiments, B is substituted with —CH₂CONH₂, —CH₂CON(CH₃)(H), —CONH₂ or —CH₂CH₂CONH₂.

In certain embodiments, B is substituted with haloC₁-C₆alkoxy. Suitable haloalkoxys include, but are not limited to, trifluoromethoxy, difluoromethoxy and monofluoromethoxy. In certain embodiments, B is substituted with trifluoromethoxy.

In certain embodiments, B is substituted with C₁-C₆alkylheteroaryl. Suitable examples of —C₁-C₆alkylheteroaryl, include any C₁-C₆alkyl as defined above wherein a hydrogen is replaced with a heteroaryl group. In certain embodiments, B is substituted with CH₂pyridyl, CH₂isoaxazole, CH₂pyrazole or CH₂CH₂Oxoimididazolidinyl.

In certain embodiments, B is substituted with heterocycloalkyl. Suitable cycloheteroalkyls include, but are not limited to, tetrahydropyranyl, tetrahydrofuranyl, pyrrolidinyl, piperidinyl, piperazinyl, dioxanyl, imidazolidinyl, oxetane, azetidine, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, tetrahydropyran, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. In certain embodiments, B is substituted with oxetane, tetrahydrofuranyl, azetidine, pyranyl, dioxidothietanyl, oxopyrrolidinyl, dioxidotetrahydrothiophenyl, oxomorpholinyl, oxabicyclohexanyl or

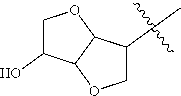 [SMILES: *C1COC2C(O)COC12]

In certain embodiments, B is substituted with —C₁-C₆alkylCN. Suitable examples of —C₁-C₆alkylCN, include any C₁-C₆alkyl as defined above wherein a hydrogen is replaced with a cyano group. In certain embodiments, B is substituted with cyanoethyl, cyanomethyl, cyanopropyl or cyanobutyl.

In certain embodiments, B is substituted with C₁-C₆alkylheterocycloalkyl. Suitable examples of —C₁-C₆alkylheterocycloalkyl, include any C₁-C₆alkyl as defined above wherein a hydrogen is replaced with a heterocycloalkyl group. In certain embodiment, B is substituted with CH₂tetrahydrofuranyl, CH₂tetrahydrofuranyl, or CH₂pyran.

In certain embodiments, B is substituted with —C₁-C₆alkylOC₁-C₆alkylOC₁-C₆alkyl. In certain embodiments, B is substituted with methoxyethoxyethyl.

In certain embodiments, B is substituted with —C₁-C₆alkylOC₁-C₆alkyl. In certain embodiments, B is substituted with methoxymethyl, ethoxyethyl, methoxypropyl or methoxybutanyl.

In certain embodiments, B is substituted with —C₁-C₆alkylCO₂C₁-C₆alkyl.

In certain embodiments, B is substituted with —C₁-C₆alkylSO₂C₁-C₆alkyl. In certain embodiments, B is substituted with CH₂SO₂CH₃.

In certain embodiments, B is substituted with halogen. Suitable halogens include, but are not limited to, a fluorine, a chlorine, a bromine or an iodine radical.

In certain embodiments, B is substituted with —C₁-C₆alkylCOOH. In certain embodiments, B is substituted with —CH₂CH₂COOH.

Additionally, in certain embodiments, B is substituted with aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, or —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, or —C₁-C₆alkylCOOH, is unsubstituted or substituted with 1 to 3 substituents independently selected from the group consisting of C₁-C₆alkyl, halogen, —NH₂, —OH, —CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl.

In certain embodiments, B is

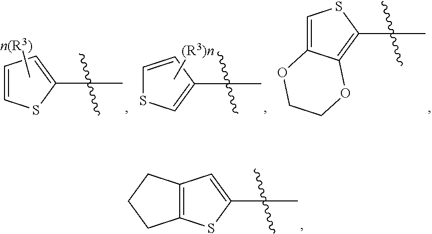

wherein each occurrence of R³ is independently —OC₁-C₆alkyl, C₁-C₆alkyl or halogen; and
n is 1 or 2.

In certain embodiments, R³ is ethoxy, methoxy, methyl or chlorine.

In certain embodiments, B is

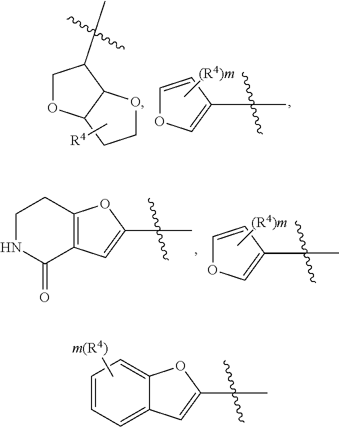

wherein each occurrence of R⁴ is independently selected from —OH, —C₁-C₆alkylOC₁-C₆alkyl, —C₁-C₆alkyl and -haloC₁-C₆alkyl; and m is 1, 2 or 3.

In certain embodiments, each occurrence of R⁴ is independently —OH, trifluoromethyl, methyl or —CH₂OCH₃.

In certain embodiments, B is

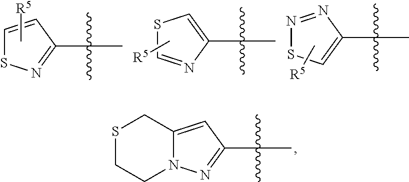

wherein R⁵ is methyl or CH₂phenyl.

In certain embodiments, B is

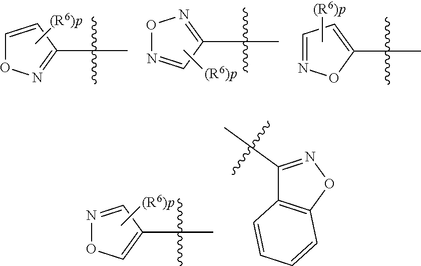

wherein each occurrence of R⁶ is independently selected from the group consisting of haloC₁-C₆alkyl, C₁-C₆alkyl, —C₁-C₆alkylOH, alkoxy, heteroaryl and C₃-C₆cycloalkyl, wherein the heteroaryl is unsubstituted or substituted with 1 to 3 C₁-C₆alkyls; and
p is 1, 2, or 3.

In certain embodiments, each occurrence of R⁶ is independently selected from the group consisting of methyl, butyl, cyclopropyl, propyl, ethanol, butanol, dimethylpyridine, methoxy and trifluorophenyl.

In certain embodiments, B is

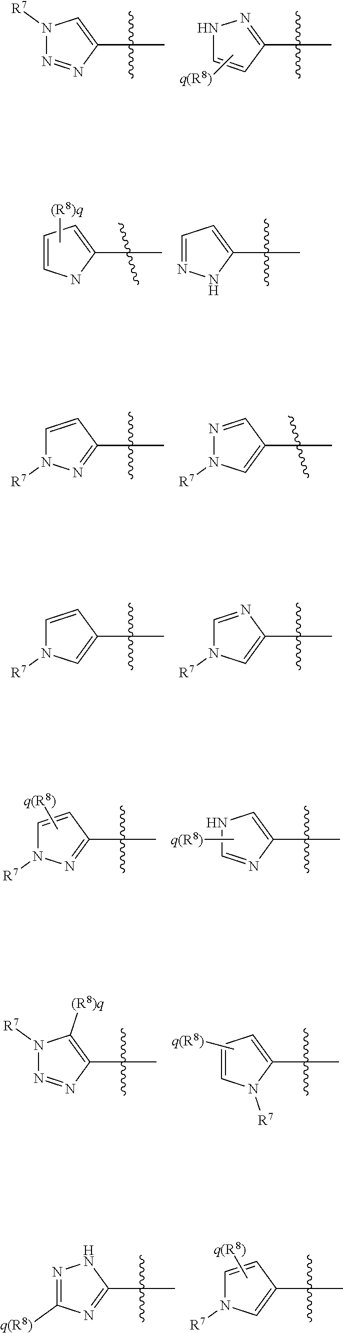

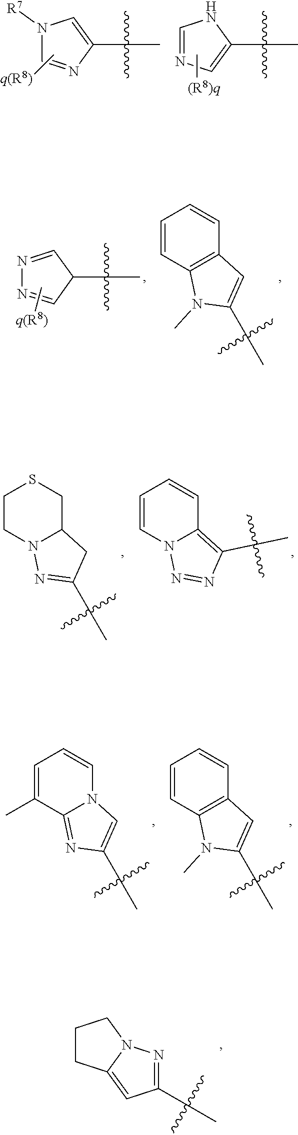

wherein R⁷ is aryl, haloC₁-C₆alkyl, C₁-C₆alkyl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl and —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, or —C₁-C₆alkylCOOH is unsubstituted or substituted with 1 to 3 substituents independently selected from the group consisting of C₁-C₆alkyl, halogen, —NH₂, —OH, CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl;
each occurrence of R¹ is independently C₁-C₆alkyl, —C₁-C₆alkylOH, C₃-C₆cycloalkyl, or halogen;
R^{a} is hydrogen, C₁-C₆alkyl or haloC₁-C₆alkyl; and
q is 1, 2, or 3.

In certain embodiments, R⁷ is dimethylphenyl, dimethylpyridyl, fluoromethyl, fluoroethyl, fluorobutyl, difluoroethyl, difluorobutyl, trifluoromethyl, trifluoroethyl, trifluorobutyl, fluoropentyl, methyl, butyl, hexyl, cyclopropyl, cyclobutyl, cyclohexane, propyl, ethanol, propanol, butanol, dimethylpyridine, methoxy, —CH₂CONH₂ and pyrazole, tetrahydrofuran, trifluorophenyl, oxetane, cyanomethyl, methylphenyl, difluorocyclobutyl, —CH₂SO₂CH₃, tetrahydropyran, pyridine, pyrimidine, pyrazine,

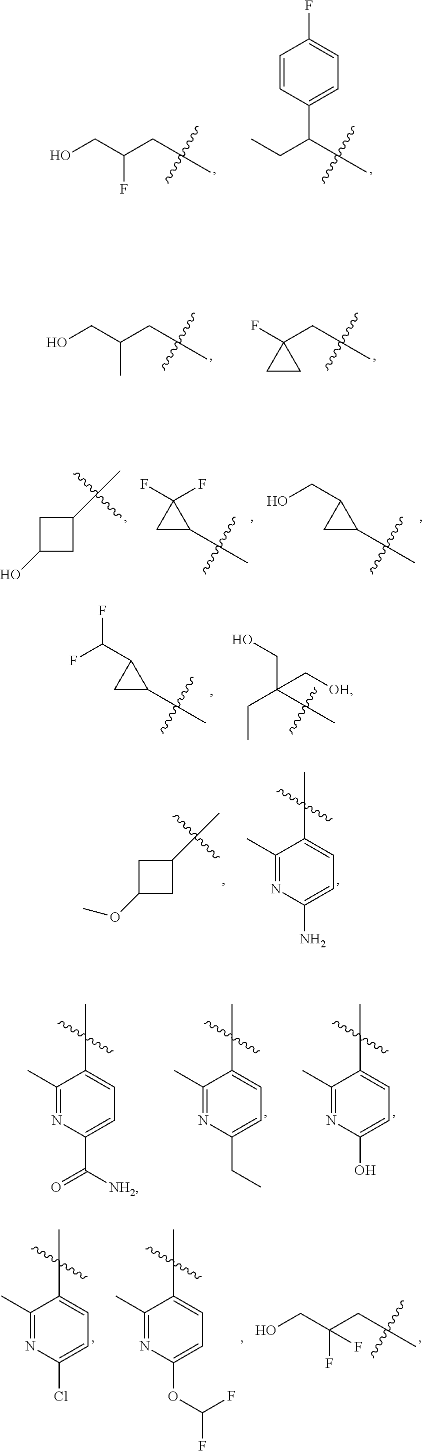

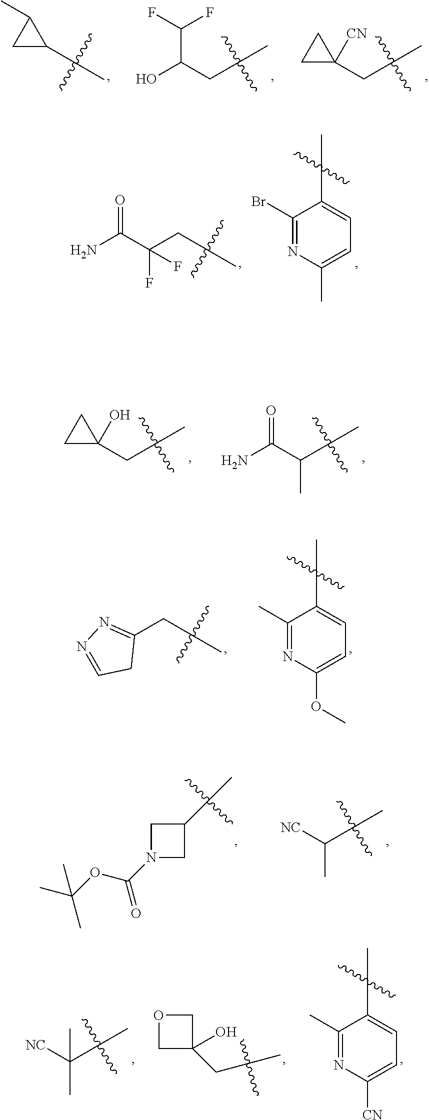

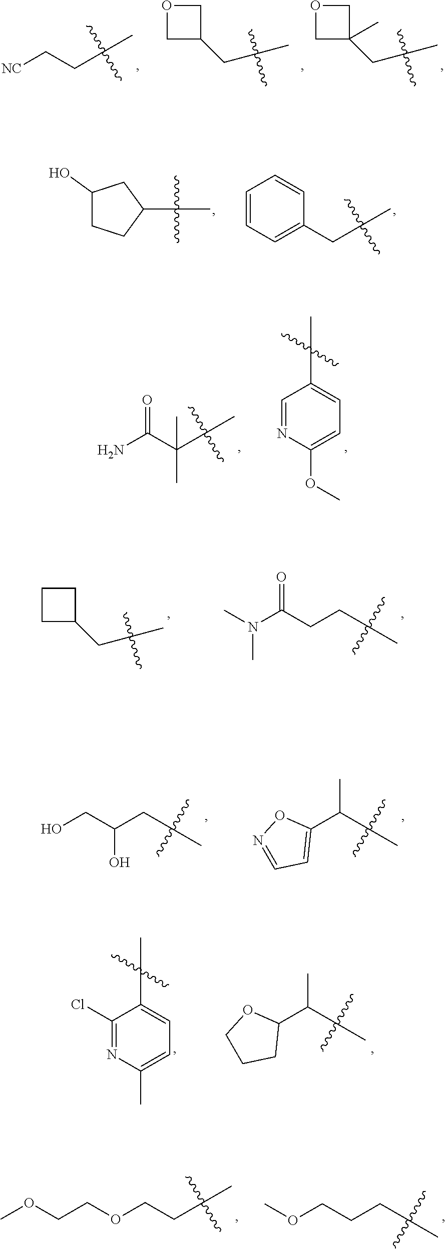

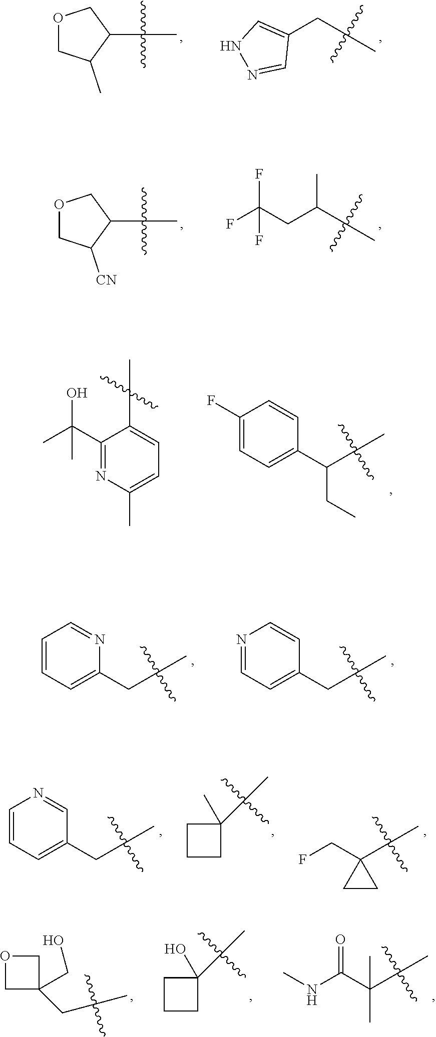

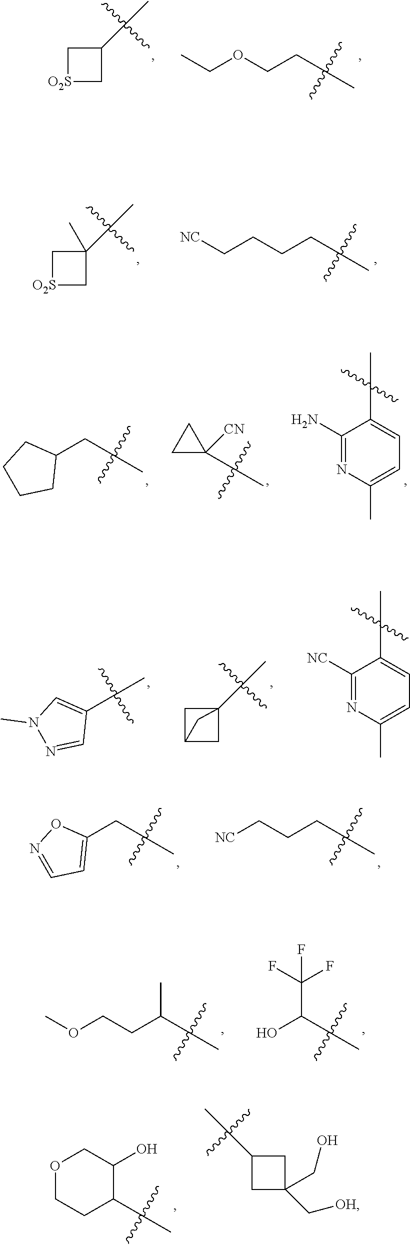

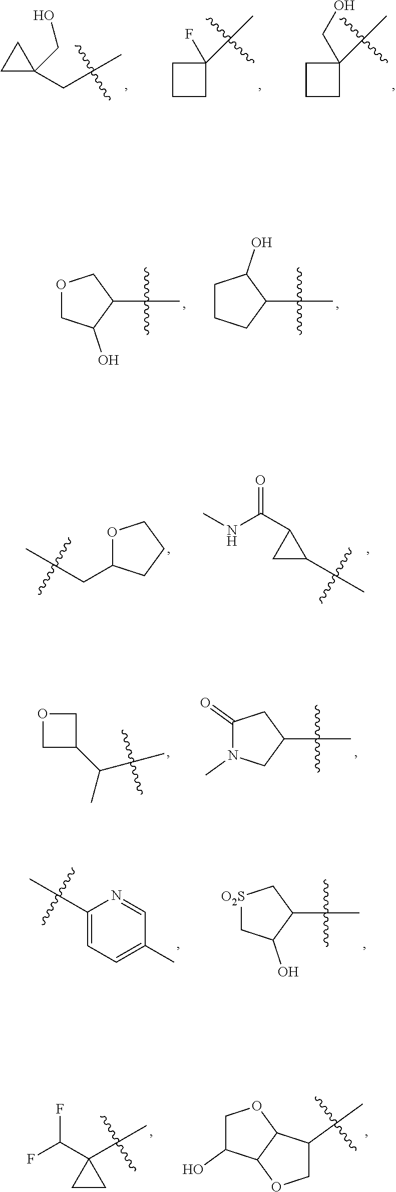

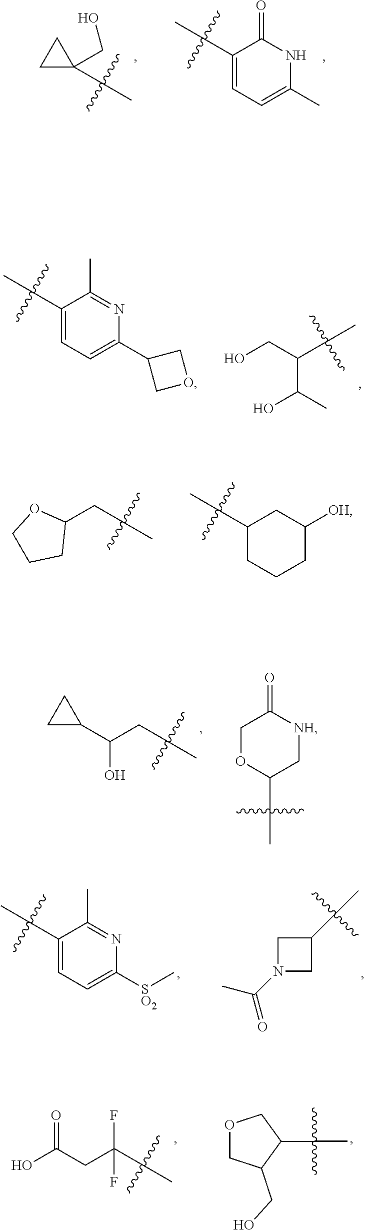

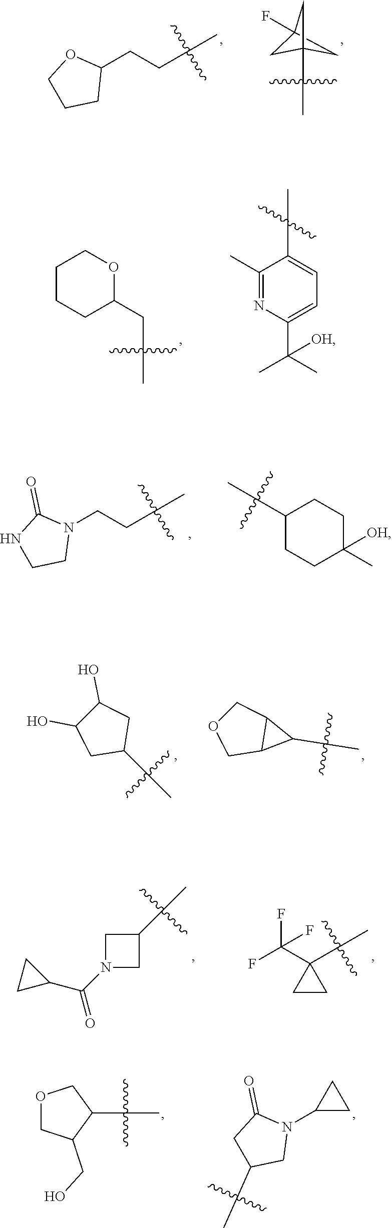

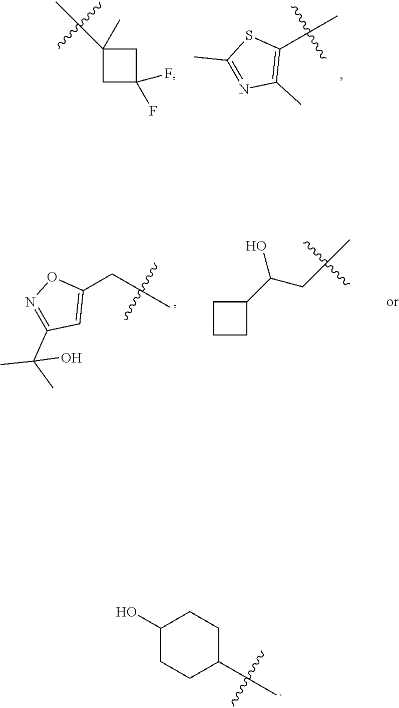

In certain embodiments, B is

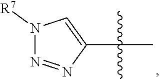

wherein R⁷ is dimethylphenyl, dimethylpyridyl, fluoromethyl, fluoroethyl, fluorobutyl, difluoroethyl, difluorobutyl, trifluoromethyl, trifluoroethyl, trifluorobutyl, fluoropentyl, methyl, butyl, hexyl, cyclopropyl, cyclobutyl, cyclohexane, propyl, ethanol, propanol, butanol, dimethylpyridine, methoxy, —CH₂CONH₂ and pyrazole, tetrahydrofuran, trifluorophenyl, oxetane, cyanomethyl, methylphenyl, difluorocyclobutyl, —CH₂SO₂CH₃, tetrahydropyran, pyridine, pyrimidine, pyrazine,

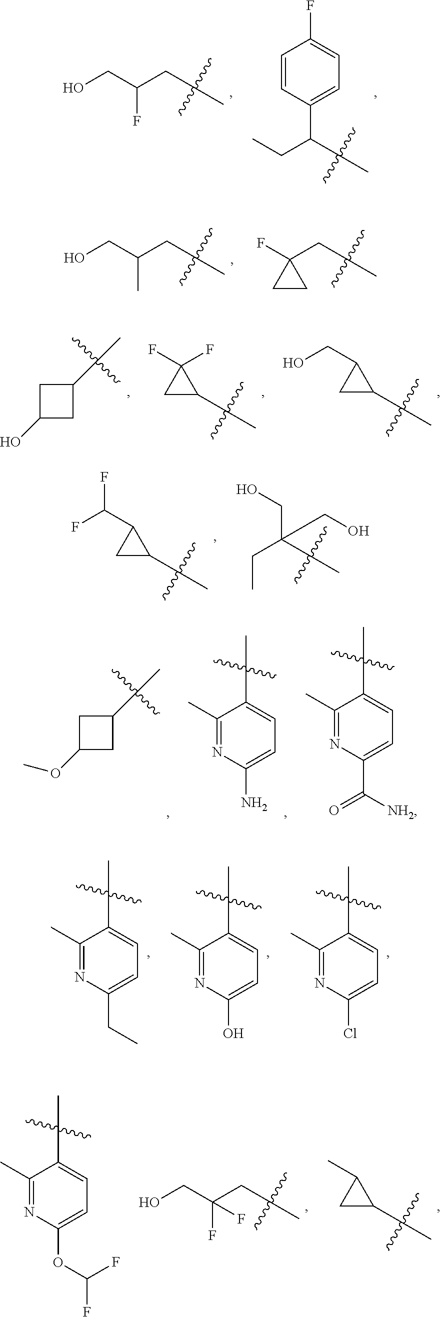

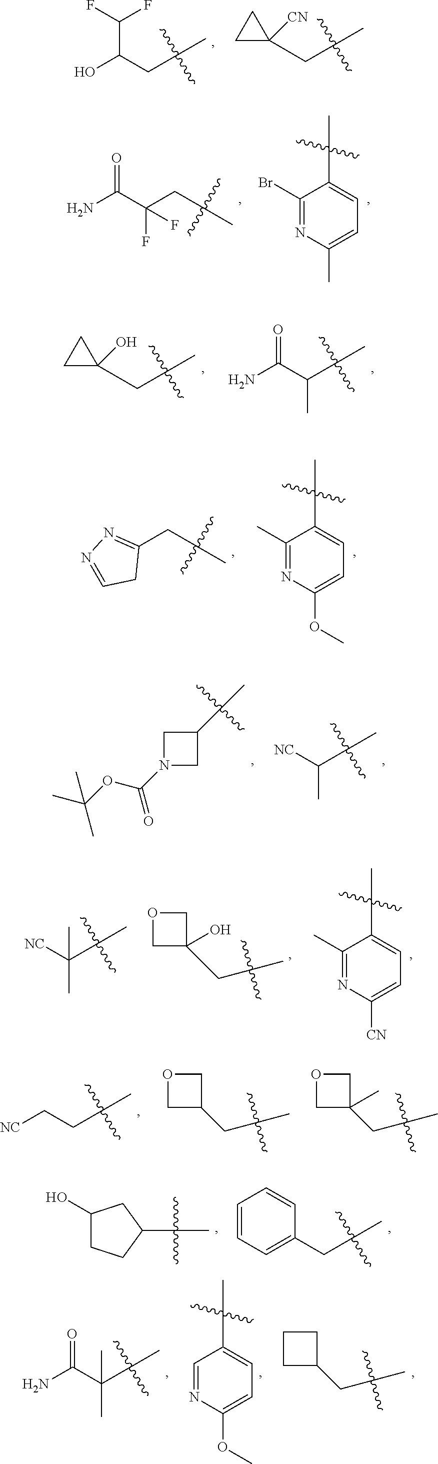

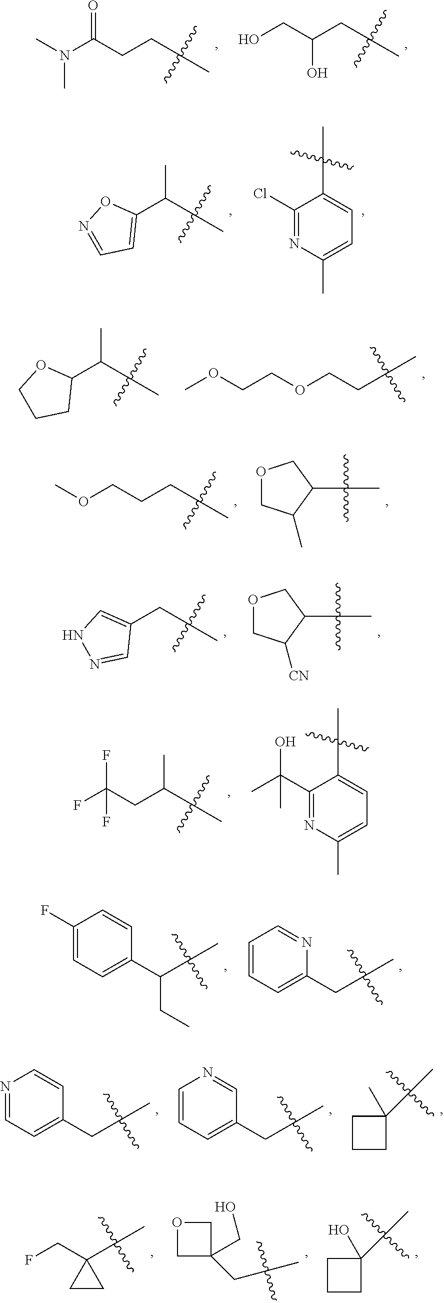

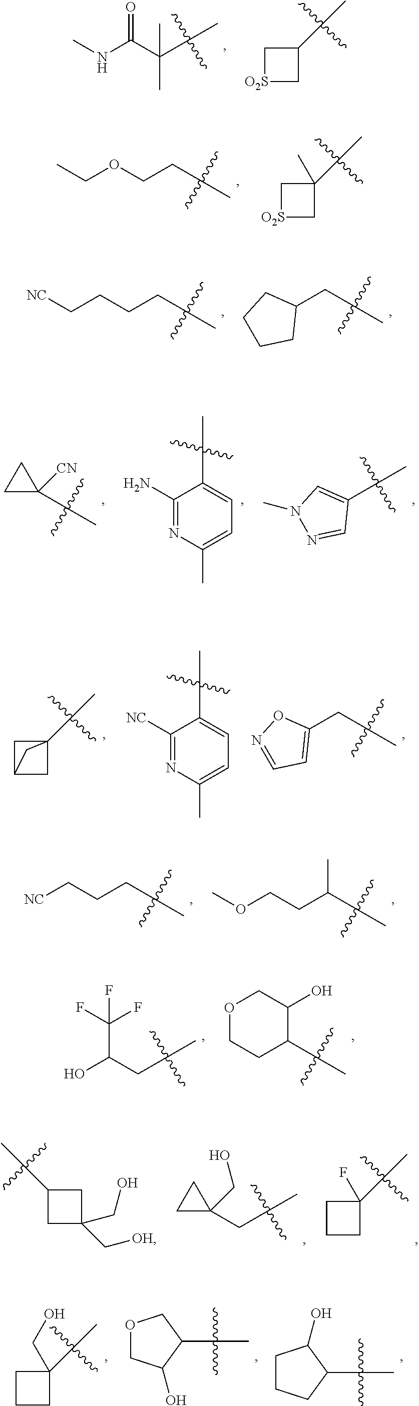

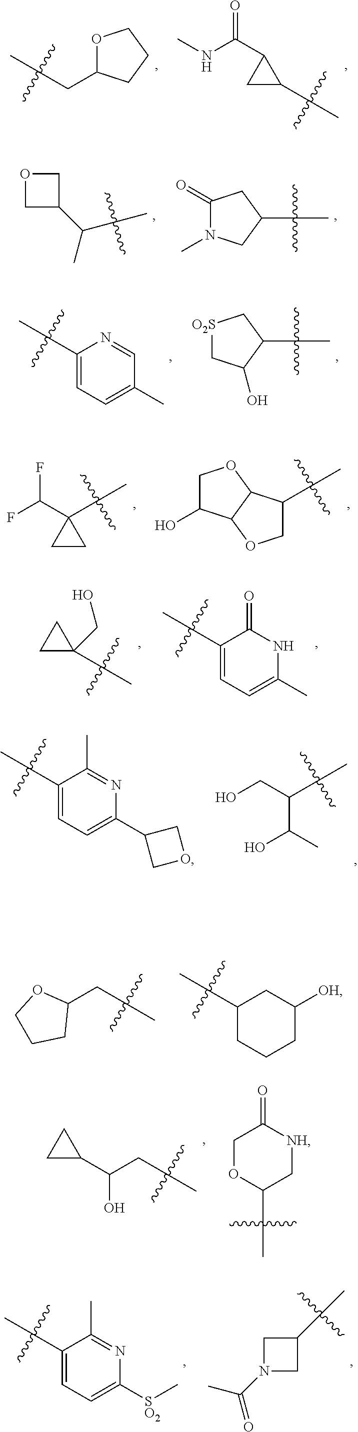

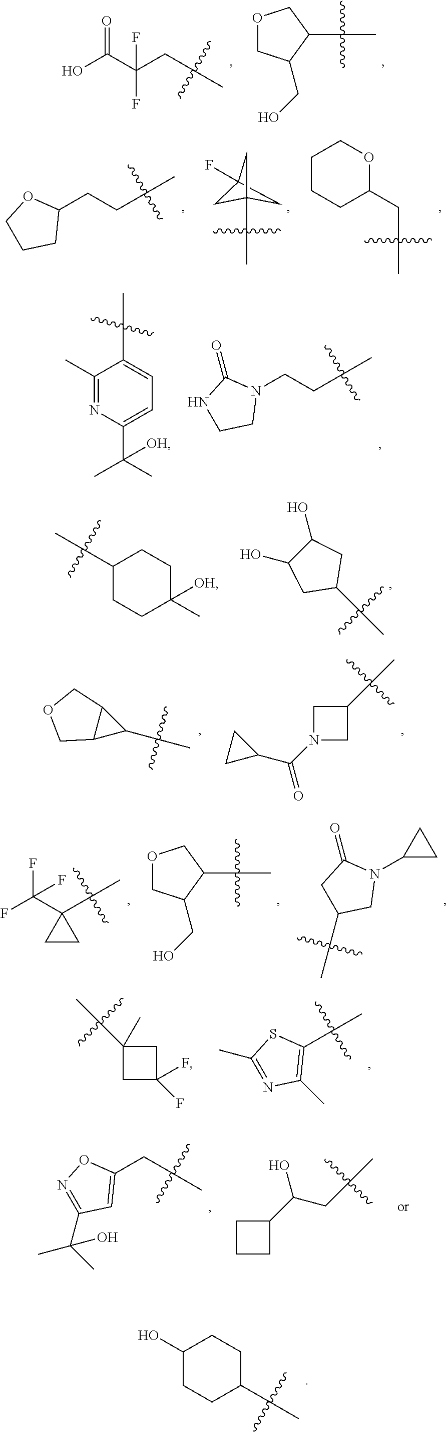

In certain embodiments, R is dimethylpyridine.

With regard to the compounds described herein, R² is hydrogen, C₁-C₆alkyl or haloC₁-C₆alkyl. In certain embodiments, R² is hydrogen. In certain embodiments, R² is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R² is methyl or ethyl. In certain embodiments, R² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

Also described herein are compounds of Formula IA:

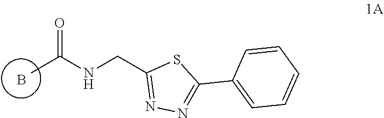

or a pharmaceutically acceptable salt thereof, wherein B is a five-membered heteroaryl, wherein five-membered heteroaryl is substituted with one to three substituents, wherein the substituents are independently selected from the group consisting of aryl, haloC₁-C₆alkyl, C₁-C₆alkyl, —C₁-C₆alkylOH, alkoxy, —OH, C₁-C₆alkylaryl, C₃-C₆cycloalkyl, C₁-C₆alkylC₃-C₆cycloalkyl, heteroaryl, —C₁-C₆alkylCON(R^{a})₂, haloC₁-C₆alkoxy, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl, halogen, and —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, and —C₁-C₆alkylCOOH is unsubstituted or substituted with one to three substituents independently selected from the group consisting of C₁-C₆alkyl, halogen, —NH₂, —OH, —CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl; and
R^{a} is hydrogen, C₁-C₆alkyl or haloC₁-C₆alkyl. All of the substituents are further defined above.

In certain embodiments, of compounds of Formula IA, or a pharmaceutically salt thereof, B is

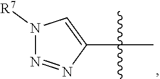

wherein R⁷ is dimethylphenyl, fluoromethyl, fluoroethyl, fluorobutyl, difluoroethyl, difluorobutyl, trifluoromethyl, trifluoroethyl, trifluorobutyl, fluoropentyl, methyl, butyl, hexyl, cyclopropyl, cyclobutyl, cyclohexane, propyl, ethanol, propanol, butanol, dimethylpyridine, methoxy, —CH₂CONH₂ and pyrazole, tetrahydrofuran, trifluorophenyl, oxetane, cyanomethyl, methylphenyl, difluorocyclobutyl, —CH₂SO₂CH₃, tetrahydropyran, pyridine, pyrimidine, pyrazine,

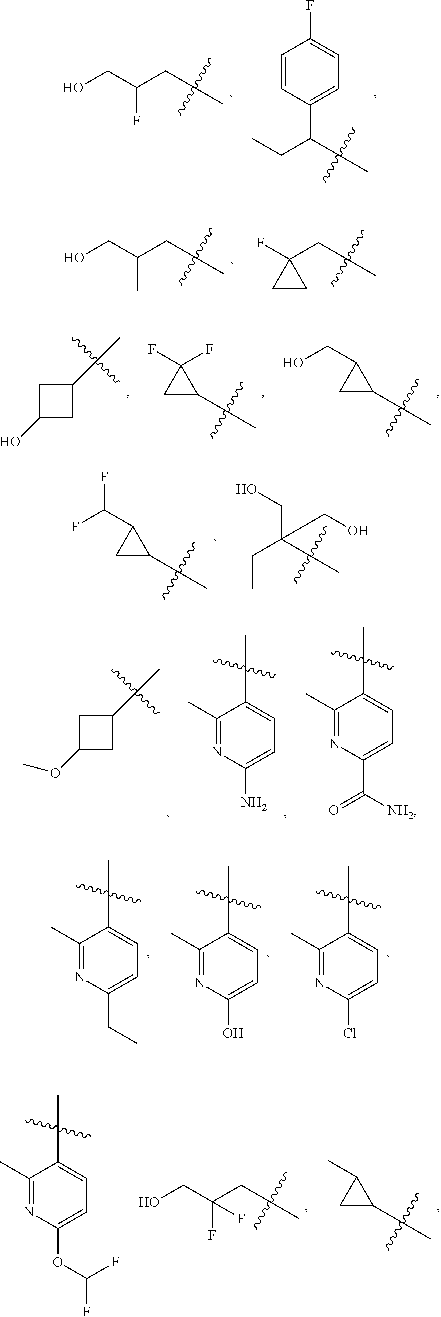

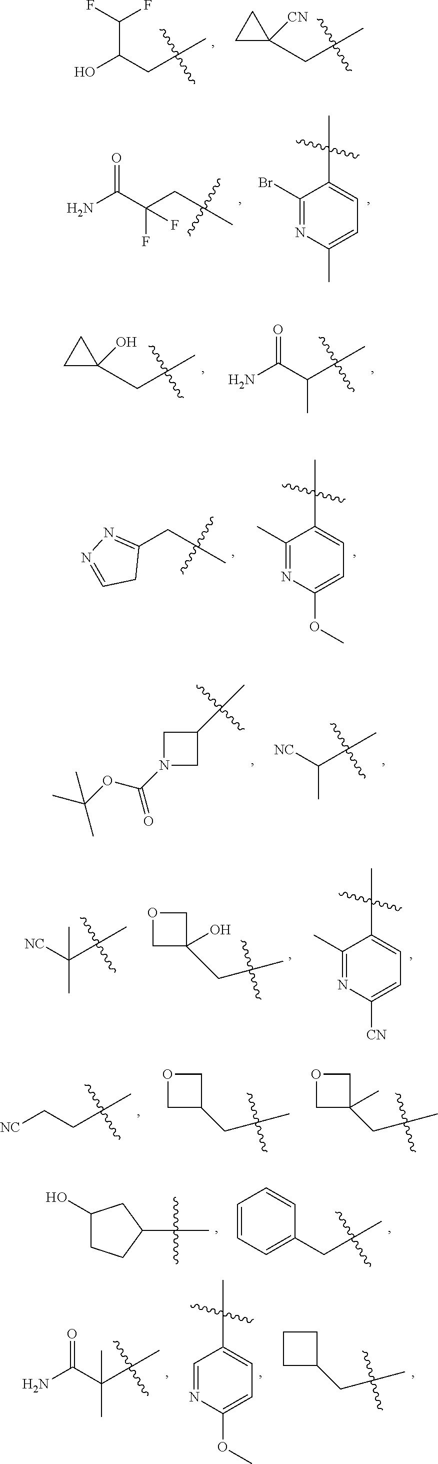

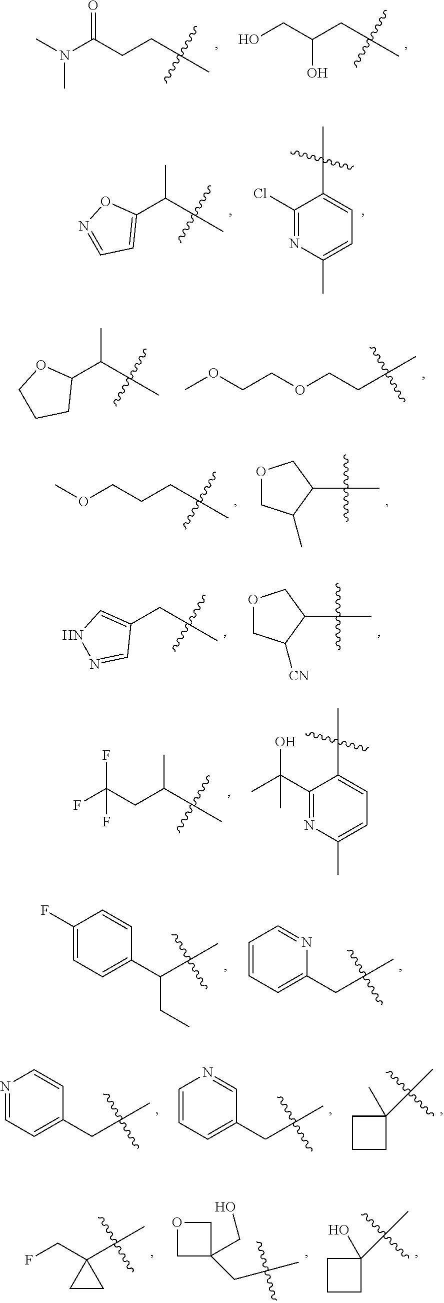

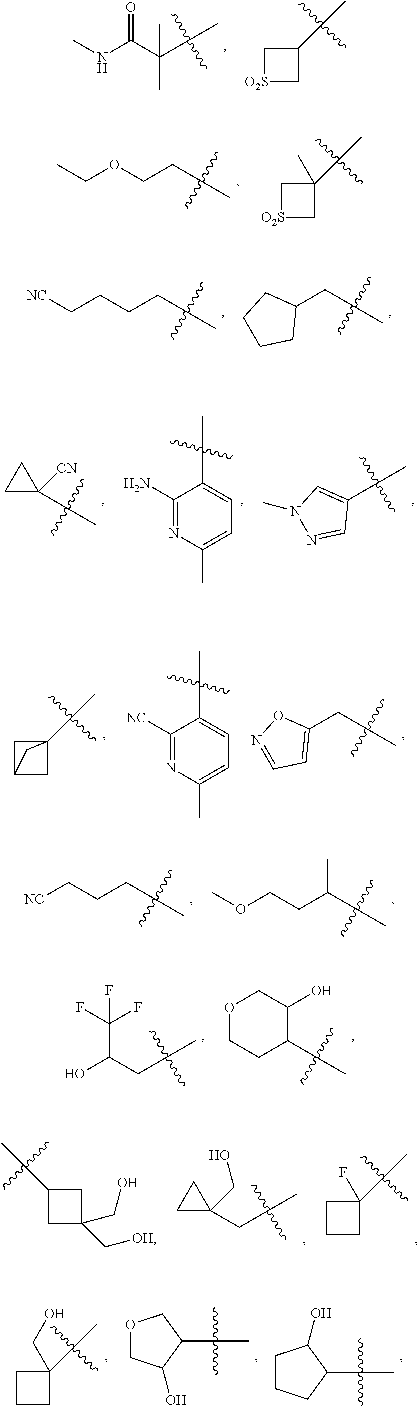

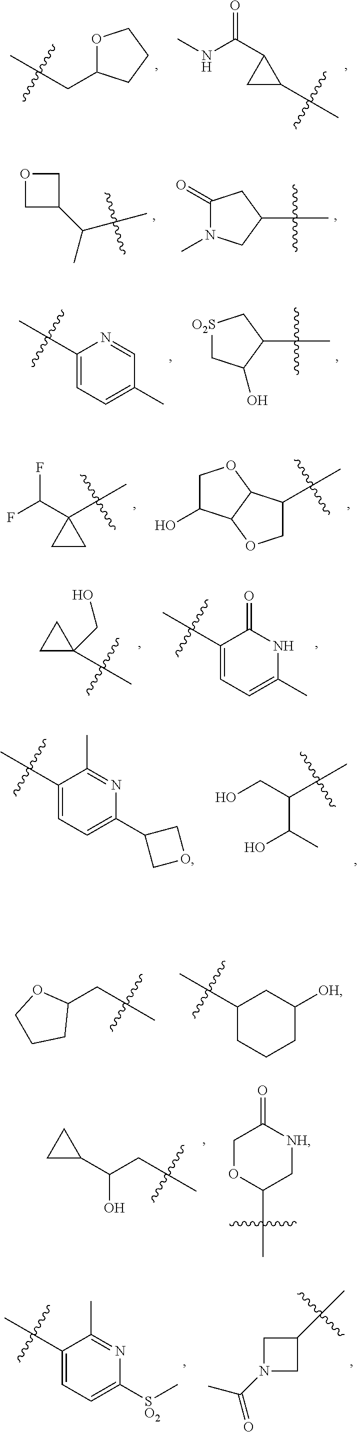

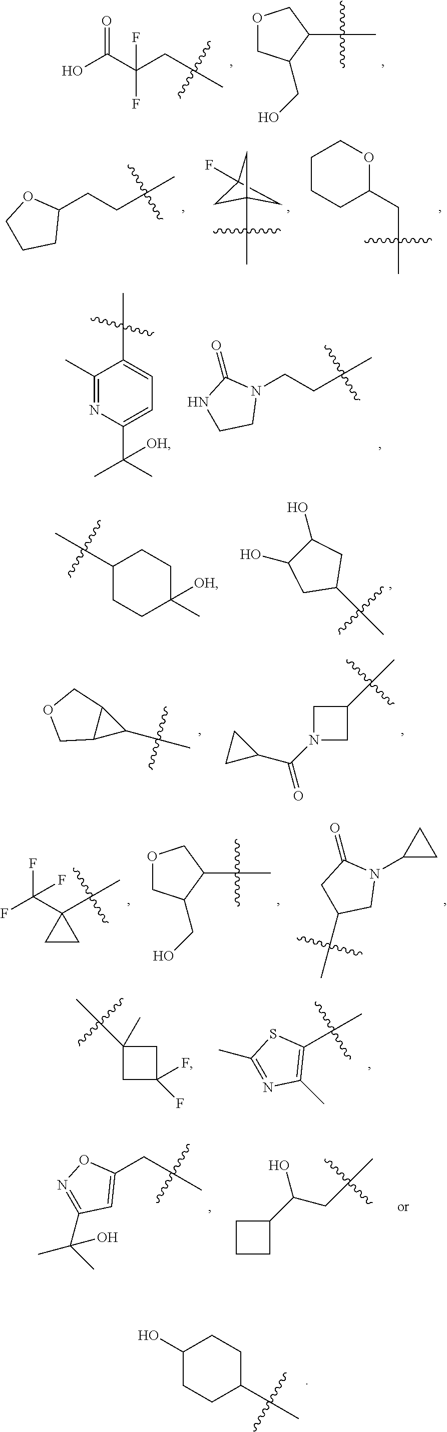

In certain embodiments of any of the compounds described above, R⁷ is selected from the group consisting of:

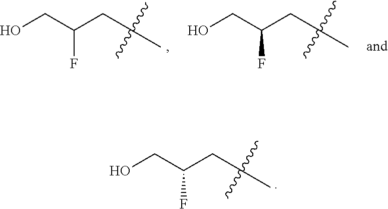 [SMILES: *CC(F)CO ; *C[C@@H](F)CO ; *C[C@H](F)CO]

Also described herein are compounds having the following structure:

| Structure | Name |
|---|---|
| 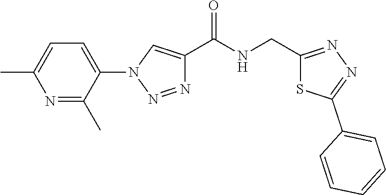 [SMILES: Cc1ccc(-n2cc(C(=O)NCc3nnc(-c4ccccc4)s3)nn2)c(C)n1] | 1-(2,6-dimethylpyridin-3-yl)-N-((5-phenyl-1,3,4- thiadiazol-2-yl)methyl)-1H-1,2,3-triazole-4- carboxamide |
| 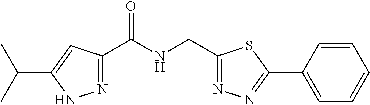 [SMILES: CC(C)c1cc(C(=O)NCc2nnc(-c3ccccc3)s2)n[nH]1] | 5-isopropyl-N-[(5-phenyl-1,3,4-thiadiazol-2- yl)methyl]-1H-pyrazole-3-carboxamide |
| 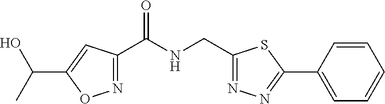 [SMILES: CC(O)c1cc(C(=O)NCc2nnc(-c3ccccc3)s2)no1] | 5-(1-hydroxyethyl)-N-[(5-phenyl-1,3,4-thiadiazol- 2-yl)methyl]isoxazole-3-carboxamide |
| 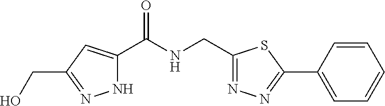 [SMILES: O=C(NCc1nnc(-c2ccccc2)s1)c1cc(CO)n[nH]1] | 3-(hydroxymethyl)-N-((5-phenyl-1,3,4-thiadiazol- 2-yl)methyl)-1H-pyrazole-5-carboxamide |
| 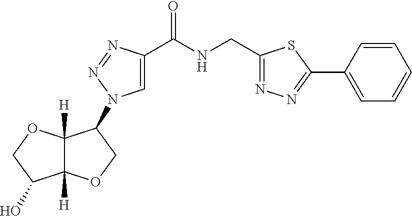 [SMILES: O=C(NCc1nnc(-c2ccccc2)s1)c1cn([C@H]2CO[C@H]3[C@@H]2OC[C@H]3O)nn1] | 1-[(3S,3aR,6R,6aR)-6-hydroxy-2,3,3a,5,6,6a- hexahydrofuro[3,2-b]furan-3-yl]-N-[(5-phenyl- 1,3,4-thiadiazol-2-yl)methyl]triazole-4- carboxamide |
| 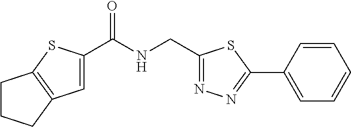 [SMILES: O=C(NCc1nnc(-c2ccccc2)s1)c1cc2c(s1)CCC2] | N-[(5-phenyl-1,3,4-thiadiazol-2-yl)methyl]-5,6- dihydro-4H-cyclopenta[b]thiophene-2-carboxamide |
| 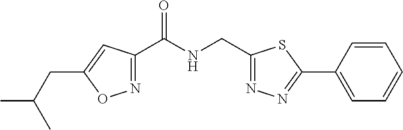 [SMILES: CC(C)Cc1cc(C(=O)NCc2nnc(-c3ccccc3)s2)no1] | 5-isobutyl-N-[(5-phenyl-1,3,4-thiadiazol-2- yl)methyl]isoxazole-3-carboxamide |
| 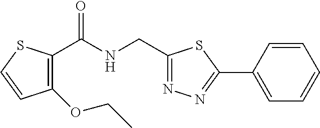 [SMILES: CCOc1ccsc1C(=O)NCc1nnc(-c2ccccc2)s1] | 3-ethoxy-N-[(5-phenyl-1,3,4-thiadiazol-2- yl)methyl]thiophene-2-carboxamide |
| 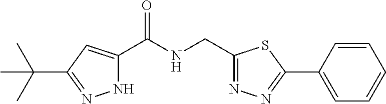 [SMILES: CC(C)(C)c1cc(C(=O)NCc2nnc(-c3ccccc3)s2)[nH]n1] | 3-(tert-butyl)-N-((5-phenyl-1,3,4-thiadiazol-2- yl)methyl)-1H-pyrazole-5-carboxamide |
| 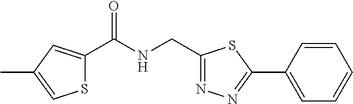 [SMILES: Cc1csc(C(=O)NCc2nnc(-c3ccccc3)s2)c1] | 4-methyl-N-[(5-phenyl-1,3,4-thiadiazol-2- yl)methyl]thiophene-2-carboxamide |
| 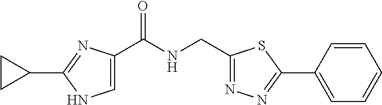 [SMILES: O=C(NCc1nnc(-c2ccccc2)s1)c1c[nH]c(C2CC2)n1] | 2-cyclopropyl-N-((5-phenyl-1,3,4-thiadiazol-2- yl)methyl)-1H-imidazole-4-carboxamide |
| 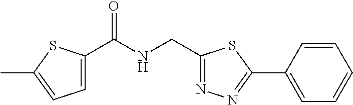 [SMILES: Cc1ccc(C(=O)NCc2nnc(-c3ccccc3)s2)s1] | 5-methyl-N-[(5-phenyl-1,3,4-thiadiazol-2- yl)methyl]thiophene-2-carboxamide |
| 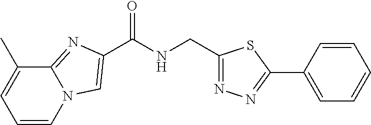 [SMILES: Cc1cccn2cc(C(=O)NCc3nnc(-c4ccccc4)s3)nc12] | 8-methyl-N-((5-phenyl-1,3,4-thiadiazol-2- yl)methyl)imidazo[1,2-a]pyridine-2-carboxamide |
| 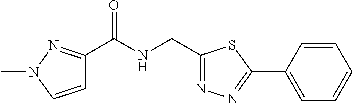 [SMILES: Cn1ccc(C(=O)NCc2nnc(-c3ccccc3)s2)n1] | 1-methyl-N-[(5-phenyl-1,3,4-thiadiazol-2- yl)methyl]pyrazole-3-carboxamide |
| 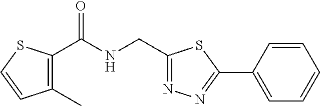 [SMILES: Cc1ccsc1C(=O)NCc1nnc(-c2ccccc2)s1] | 3-methyl-N-[(5-phenyl-1,3,4-thiadiazol-2- yl)methyl]thiophene-2-carboxamide |
| 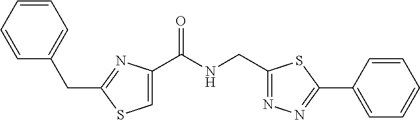 [SMILES: O=C(NCc1nnc(-c2ccccc2)s1)c1csc(Cc2ccccc2)n1] | 2-benzyl-N-((5-phenyl-1,3,4-thiadiazol-2- yl)methyl)thiazole-4-carboxamide |
| 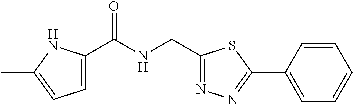 [SMILES: Cc1ccc(C(=O)NCc2nnc(-c3ccccc3)s2)[nH]1] | 5-methyl-N-[(5-phenyl-1,3,4-thiadiazol-2- yl)methyl]-1H-pyrrole-2-carboxamide |
| 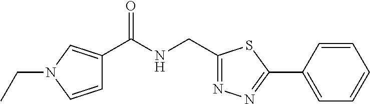 [SMILES: CCn1ccc(C(=O)NCc2nnc(-c3ccccc3)s2)c1] | 1-ethyl-N-[(5-phenyl-1,3,4-thiadiazol-2- yl)methyl]pyrrole-3-carboxamide |
| 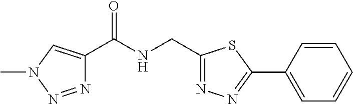 [SMILES: Cn1cc(C(=O)NCc2nnc(-c3ccccc3)s2)nn1] | 1-methyl-N-[(5-phenyl-1,3,4-thiadiazol-2- yl)methyl]triazole-4-carboxamide |
| 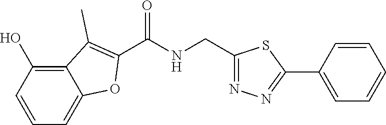 [SMILES: Cc1c(C(=O)NCc2nnc(-c3ccccc3)s2)oc2cccc(O)c12] | 4-hydroxy-3-methyl-N-[(5-phenyl-1,3,4-thiadiazol- 2-yl)methyl]benzofuran-2-carboxamide |
| 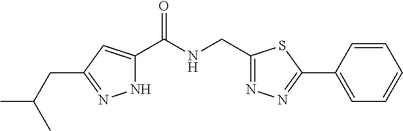 [SMILES: CC(C)Cc1cc(C(=O)NCc2nnc(-c3ccccc3)s2)[nH]n1] | 3-isobutyl-N-((5-phenyl-1,3,4-thiadiazol-2- yl)methyl)-1H-pyrazole-5-carboxamide |
| 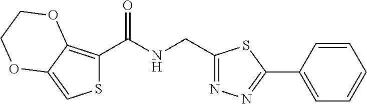 [SMILES: O=C(NCc1nnc(-c2ccccc2)s1)c1scc2c1OCCO2] | N-[(5-phenyl-1,3,4-thiadiazol-2-yl)methyl]-2,3- dihydrothieno[3,4-b][1,4]dioxine-5-carboxamide |
| 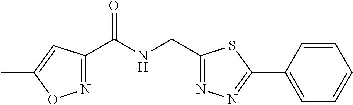 [SMILES: Cc1cc(C(=O)NCc2nnc(-c3ccccc3)s2)no1] | 5-methyl-N-[(5-phenyl-1,3,4-thiadiazol-2- yl)methyl]isoxazole-3-carboxamide |
| 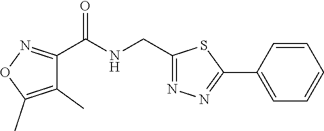 [SMILES: Cc1onc(C(=O)NCc2nnc(-c3ccccc3)s2)c1C] | 4,5-dimethyl-N-[(5-phenyl-1,3,4-thiadiazol-2- yl)methyl]isoxazole-3-carboxamide |
| 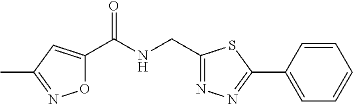 [SMILES: Cc1cc(C(=O)NCc2nnc(-c3ccccc3)s2)on1] | 3-methyl-N-[(5-phenyl-1,3,4-thiadiazol-2- yl)methyl]isoxazole-5-carboxamide |
| 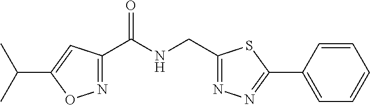 [SMILES: CC(C)c1cc(C(=O)NCc2nnc(-c3ccccc3)s2)no1] | 5-isopropyl-N-[(5-phenyl-1,3,4-thiadiazol-2- yl)methyl]isoxazole-3-carboxamide |
| 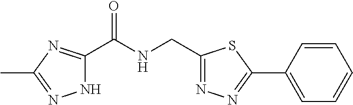 [SMILES: Cc1n[nH]c(C(=O)NCc2nnc(-c3ccccc3)s2)n1] | 3-methyl-N-((5-phenyl-1,3,4-thiadiazol-2- yl)methyl)-1H-1,2,4-triazole-5-carboxamide |
| 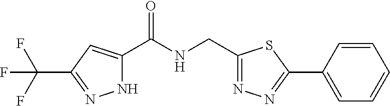 [SMILES: O=C(NCc1nnc(-c2ccccc2)s1)c1cc(C(F)(F)F)n[nH]1] | N-[(5-phenyl-1,3,4-thiadiazol-2-yl)methyl]-3- (trifluoromethyl)-1H-pyrazole-5-carboxamide |
| 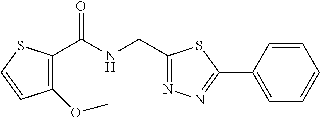 [SMILES: COc1ccsc1C(=O)NCc1nnc(-c2ccccc2)s1] | 3-methoxy-N-[(5-phenyl-1,3,4-thiadiazol-2- yl)methyl]thiophene-2-carboxamide |
| 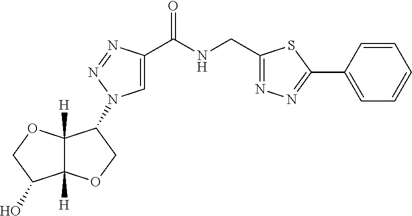 [SMILES: O=C(NCc1nnc(-c2ccccc2)s1)c1cn([C@@H]2CO[C@H]3[C@@H]2OC[C@H]3O)nn1] | 1-((3R,3aR,6R,6aR)-6-hydroxyhexahydrofuro[3,2- b]furan-3-yl)-N-((5-phenyl-1,3,4-thiadiazol-2- yl)methyl)-1H-1,2,3-triazole-4-carboxamide |
| 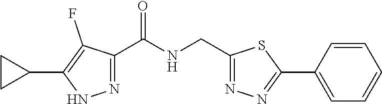 [SMILES: O=C(NCc1nnc(-c2ccccc2)s1)c1n[nH]c(C2CC2)c1F] | 5-cyclopropyl-4-fluoro-N-[(5-phenyl-1,3,4- thiadiazol-2-yl)methyl]-1H-pyrazole-3- carboxamide |
| 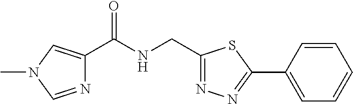 [SMILES: Cn1cnc(C(=O)NCc2nnc(-c3ccccc3)s2)c1] | 1-methyl-N-((5-phenyl-1,3,4-thiadiazol-2- yl)methyl)-1H-imidazole-4-carboxamide |
| 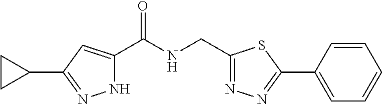 [SMILES: O=C(NCc1nnc(-c2ccccc2)s1)c1cc(C2CC2)n[nH]1] | 3-cyclopropyl-N-[(5-phenyl-1,3,4-thiadiazol-2- yl)methyl]-1H-pyrazole-5-carboxamide |
| 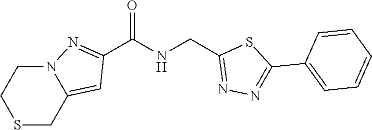 [SMILES: O=C(NCc1nnc(-c2ccccc2)s1)c1cc2n(n1)CCSC2] | N-[(5-phenyl-1,3,4-thiadiazol-2-yl)methyl]-6,7- dihydro-4H-pyrazolo[5,1-c][1,4]thiazine-2- carboxamide |
| 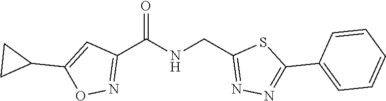 [SMILES: O=C(NCc1nnc(-c2ccccc2)s1)c1cc(C2CC2)on1] | 5-cyclopropyl-N-[(5-phenyl-1,3,4-thiadiazol-2- yl)methyl]isoxazole-3-carboxamide |
| 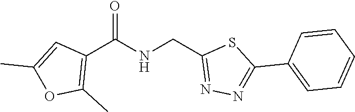 [SMILES: Cc1cc(C(=O)NCc2nnc(-c3ccccc3)s2)c(C)o1] | 2,5-dimethyl-N-[(5-phenyl-1,3,4-thiadiazol-2- yl)methyl]furan-3-carboxamide |
| 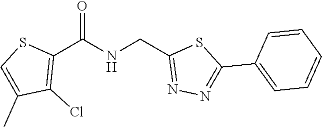 [SMILES: Cc1csc(C(=O)NCc2nnc(-c3ccccc3)s2)c1Cl] | 3-chloro-4-methyl-N-[(5-phenyl-1,3,4-thiadiazol-2- yl)methyl]thiophene-2-carboxamide |
| 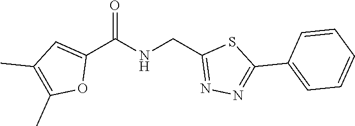 [SMILES: Cc1cc(C(=O)NCc2nnc(-c3ccccc3)s2)oc1C] | 4,5-dimethyl-N-[(5-phenyl-1,3,4-thiadiazol-2- yl)methyl]furan-2-carboxamide |
| 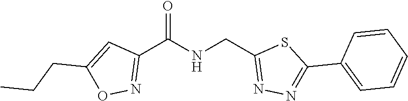 [SMILES: CCCc1cc(C(=O)NCc2nnc(-c3ccccc3)s2)no1] | N-[(5-phenyl-1,3,4-thiadiazol-2-yl)methyl]-5- propyl-isoxazole-3-carboxamide |
| 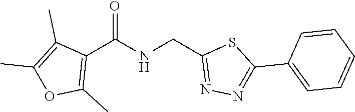 [SMILES: Cc1oc(C)c(C(=O)NCc2nnc(-c3ccccc3)s2)c1C] | 2,4,5-trimethyl-N-[(5-phenyl-1,3,4-thiadiazol-2- yl)methyl]furan-3-carboxamide |
| 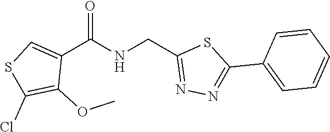 [SMILES: COc1c(C(=O)NCc2nnc(-c3ccccc3)s2)csc1Cl] | 5-chloro-4-methoxy-N-[(5-phenyl-1,3,4-thiadiazol- 2-yl)methyl]thiophene-3-carboxamide |
| 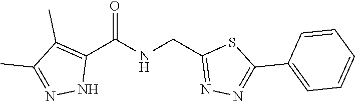 [SMILES: Cc1n[nH]c(C(=O)NCc2nnc(-c3ccccc3)s2)c1C] | 3,4-dimethyl-N-((5-phenyl-1,3,4-thiadiazol-2- yl)methyl)-1H-pyrazole-5-carboxamide |
| 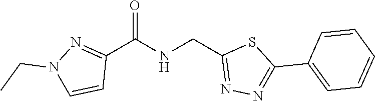 [SMILES: CCn1ccc(C(=O)NCc2nnc(-c3ccccc3)s2)n1] | 1-ethyl-N-[(5-phenyl-1,3,4-thiadiazol-2- yl)methyl]pyrazole-4-carboxamide |
| 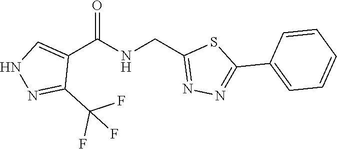 [SMILES: O=C(NCc1nnc(-c2ccccc2)s1)c1c[nH]nc1C(F)(F)F] | N-[(5-phenyl-1,3,4-thiadiazol-2-yl)methyl]-3- (trifluoromethyl)-1H-pyrazole-4-carboxamide |
| 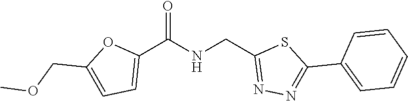 [SMILES: COCc1ccc(C(=O)NCc2nnc(-c3ccccc3)s2)o1] | 5-(methoxymethyl)-N-[(5-phenyl-1,3,4-thiadiazol- 2-yl)methyl]furan-2-carboxamide |
| 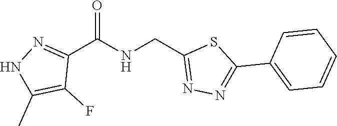 [SMILES: Cc1[nH]nc(C(=O)NCc2nnc(-c3ccccc3)s2)c1F] | 4-fluoro-5-methyl-N-[(5-phenyl-1,3,4-thiadiazol-2- yl)methyl]-1H-pyrazole-3-carboxamide |
| 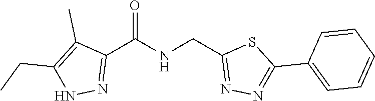 [SMILES: CCc1[nH]nc(C(=O)NCc2nnc(-c3ccccc3)s2)c1C] | 5-ethyl-4-methyl-N-((5-phenyl-1,3,4-thiadiazol-2- yl)methyl)-1H-pyrazole-3-carboxamide |
| 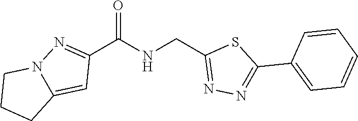 [SMILES: O=C(NCc1nnc(-c2ccccc2)s1)c1cc2n(n1)CCC2] | N-[(5-phenyl-1,3,4-thiadiazol-2-yl)methyl]-5,6- dihydro-4H-pyrrolo[1,2-b]pyrazole-2-carboxamide |
| 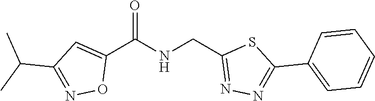 [SMILES: CC(C)c1cc(C(=O)NCc2nnc(-c3ccccc3)s2)on1] | 3-isopropyl-N-[(5-phenyl-1,3,4-thiadiazol-2- yl)methyl]isoxazole-5-carboxamide |
| 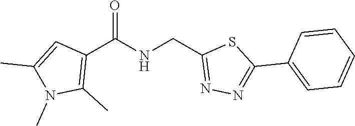 [SMILES: Cc1cc(C(=O)NCc2nnc(-c3ccccc3)s2)c(C)n1C] | 1,2,5-trimethyl-N-[(5-phenyl-1,3,4-thiadiazol-2- yl)methyl]pyrrole-3-carboxamide |
| 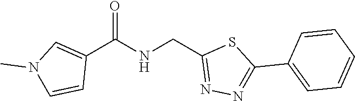 [SMILES: Cn1ccc(C(=O)NCc2nnc(-c3ccccc3)s2)c1] | 1-methyl-N-[(5-phenyl-1,3,4-thiadiazol-2- yl)methyl]pyrrole-3-carboxamide |
| 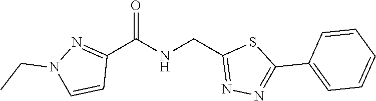 [SMILES: CCn1ccc(C(=O)NCc2nnc(-c3ccccc3)s2)n1] | 1-ethyl-N-[(5-phenyl-1,3,4-thiadiazol-2- yl)methyl]pyrazole-3-carboxamide |
| 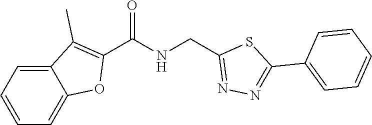 [SMILES: Cc1c(C(=O)NCc2nnc(-c3ccccc3)s2)oc2ccccc12] | 3-methyl-N-[(5-phenyl-1,3,4-thiadiazol-2- yl)methyl]benzofuran-2-carboxamide |
| 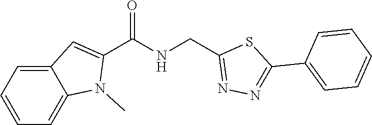 [SMILES: Cn1c(C(=O)NCc2nnc(-c3ccccc3)s2)cc2ccccc21] | 1-methyl-N-((5-phenyl-1,3,4-thiadiazol-2- yl)methyl)-1H-indole-2-carboxamide |
| 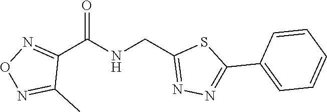 [SMILES: Cc1nonc1C(=O)NCc1nnc(-c2ccccc2)s1] | 4-methyl-N-[(5-phenyl-1,3,4-thiadiazol-2- yl)methyl]-1,2,5-oxadiazole-3-carboxamide |
| 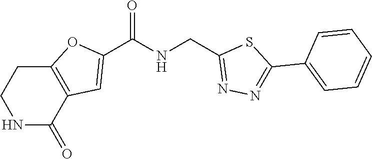 [SMILES: O=C(NCc1nnc(-c2ccccc2)s1)c1cc2c(o1)CCNC2=O] | 4-oxo-N-[(5-phenyl-1,3,4-thiadiazol-2-yl)methyl]- 6,7-dihydro-5H-furo[3,2-c]pyridine-2-carboxamide |
| 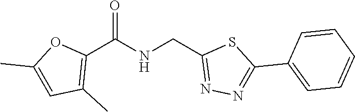 [SMILES: Cc1cc(C)c(C(=O)NCc2nnc(-c3ccccc3)s2)o1] | 3,5-dimethyl-N-[(5-phenyl-1,3,4-thiadiazol-2- yl)methyl]furan-2-carboxamide |
| 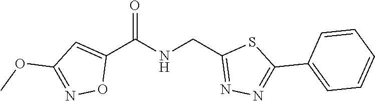 [SMILES: COc1cc(C(=O)NCc2nnc(-c3ccccc3)s2)on1] | 3-methoxy-N-[(5-phenyl-1,3,4-thiadiazol-2- yl)methyl]isoxazole-5-carboxamide |
| 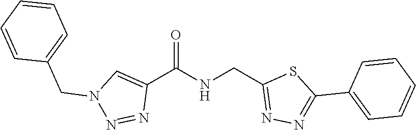 [SMILES: O=C(NCc1nnc(-c2ccccc2)s1)c1cn(Cc2ccccc2)nn1] | 1-benzyl-N-[(5-phenyl-1,3,4-thiadiazol-2- yl)methyl]triazole-4-carboxamide |
| 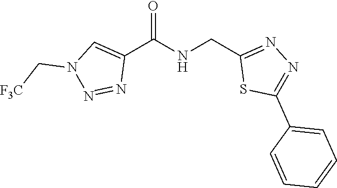 [SMILES: O=C(NCc1nnc(-c2ccccc2)s1)c1cn(CC(F)(F)F)nn1] | N-((5-phenyl-1,3,4-thiadiazol-2-yl)methyl)-1- (2,2,2-trifluoroethyl)-1H-1,2,3-triazole-4- carboxamide |
| 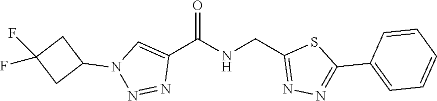 [SMILES: O=C(NCc1nnc(-c2ccccc2)s1)c1cn(C2CC(F)(F)C2)nn1] | 1-(3,3-difluorocyclobutyl)-N-((5-phenyl-1,3,4- thiadiazol-2-yl)methyl)-1H-1,2,3-triazole-4- carboxamide |
| 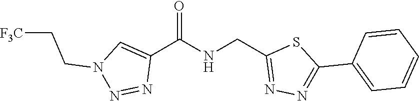 [SMILES: O=C(NCc1nnc(-c2ccccc2)s1)c1cn(CCC(F)(F)F)nn1] | N-((5-phenyl-1,3,4-thiadiazol-2-yl)methyl)-1- (3,3,3-trifluoropropyl)-1H-1,2,3-triazole-4- carboxamide |
| 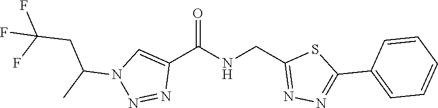 [SMILES: CC(CC(F)(F)F)n1cc(C(=O)NCc2nnc(-c3ccccc3)s2)nn1] | N-((5-phenyl-1,3,4-thiadiazol-2-yl)methyl)-1- (4,4,4-trifluorobutan-2-yl)-1H-1,2,3-triazole-4- carboxamide |
| 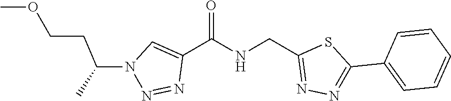 [SMILES: COCC[C@@H](C)n1cc(C(=O)NCc2nnc(-c3ccccc3)s2)nn1] | (R)-1-(4-methoxybutan-2-yl)-N-((5-phenyl-1,3,4- thiadiazol-2-yl)methyl)-1H-1,2,3-triazole-4- carboxamide |
| 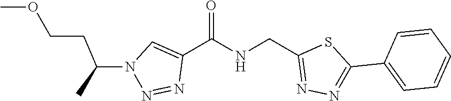 [SMILES: COCC[C@H](C)n1cc(C(=O)NCc2nnc(-c3ccccc3)s2)nn1] | (S)-1-(4-methoxybutan-2-yl)-N-((5-phenyl-1,3,4- thiadiazol-2-yl)methyl)-1H-1,2,3-triazole-4- carboxamide |
| 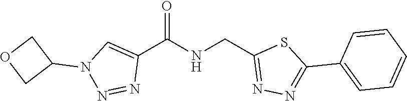 [SMILES: O=C(NCc1nnc(-c2ccccc2)s1)c1cn(C2COC2)nn1] | 1-(oxetan-3-yl)-N-((5-phenyl-1,3,4-thiadiazol-2- yl)methyl)-1H-1,2,3-triazole-4-carboxamide |
| 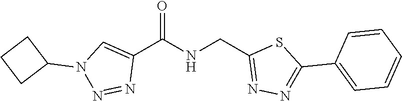 [SMILES: O=C(NCc1nnc(-c2ccccc2)s1)c1cn(C2CCC2)nn1] | 1-cyclobutyl-N-((5-phenyl-1,3,4-thiadiazol-2- yl)methyl)-1H-1,2,3-triazole-4-carboxamide |
| 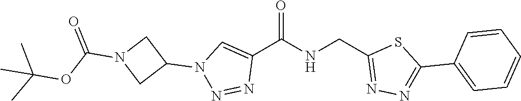 [SMILES: CC(C)(C)OC(=O)N1CC(n2cc(C(=O)NCc3nnc(-c4ccccc4)s3)nn2)C1] | tert-butyl 3-(4-(((5-phenyl-1,3,4-thiadiazol-2- yl)methyl)carbamoyl)-1H-1,2,3-triazol-1- yl)azetidine-1-carboxylate |
| 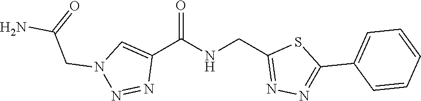 [SMILES: NC(=O)Cn1cc(C(=O)NCc2nnc(-c3ccccc3)s2)nn1] | 1-(2-amino-2-oxoethyl)-N-((5-phenyl-1,3,4- thiadiazol-2-yl)methyl)-1H-1,2,3-triazole-4- carboxamide |
| 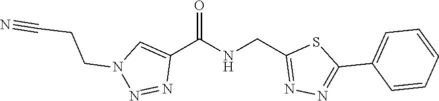 [SMILES: N#CCCn1cc(C(=O)NCc2nnc(-c3ccccc3)s2)nn1] | 1-(2-cyanoethyl)-N-((5-phenyl-1,3,4-thiadiazol-2- yl)methyl)-1H-1,2,3-triazole-4-carboxamide |
| 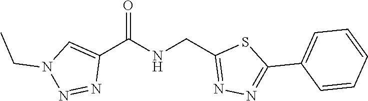 [SMILES: CCn1cc(C(=O)NCc2nnc(-c3ccccc3)s2)nn1] | 1-ethyl-N-((5-phenyl-1,3,4-thiadiazol-2-yl)methyl)- 1H-1,2,3-triazole-4-carboxamide |
| 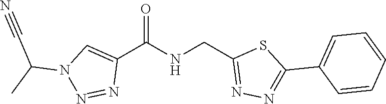 [SMILES: CC(C#N)n1cc(C(=O)NCc2nnc(-c3ccccc3)s2)nn1] | 1-(1-cyanoethyl)-N-((5-phenyl-1,3,4-thiadiazol-2- yl)methyl)-1H-1,2,3-triazole-4-carboxamide |
| 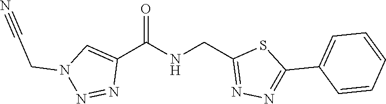 [SMILES: N#CCn1cc(C(=O)NCc2nnc(-c3ccccc3)s2)nn1] | 1-(cyanomethyl)-N-((5-phenyl-1,3,4-thiadiazol-2- yl)methyl)-1H-1,2,3-triazole-4-carboxamide |
| 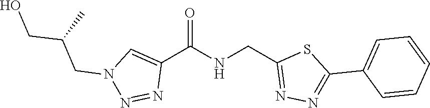 [SMILES: C[C@@H](CO)Cn1cc(C(=O)NCc2nnc(-c3ccccc3)s2)nn1] | (R)-1-(3-hydroxy-2-methylpropyl)-N-((5-phenyl- 1,3,4-thiadiazol-2-yl)methyl)-1H-1,2,3-triazole-4- carboxamide |
| 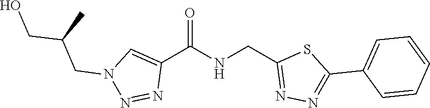 [SMILES: C[C@H](CO)Cn1cc(C(=O)NCc2nnc(-c3ccccc3)s2)nn1] | (S)-1-(3-hydroxy-2-methylpropyl)-N-((5-phenyl- 1,3,4-thiadiazol-2-yl)methyl)-1H-1,2,3-triazole-4- carboxamide |
| 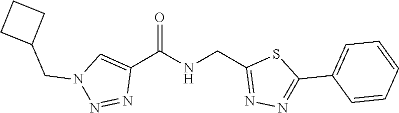 [SMILES: O=C(NCc1nnc(-c2ccccc2)s1)c1cn(CC2CCC2)nn1] | 1-(cyclobutylmethyl)-N-((5-phenyl-1,3,4- thiadiazol-2-yl)methyl)-1H-1,2,3-triazole-4- carboxamide |
| 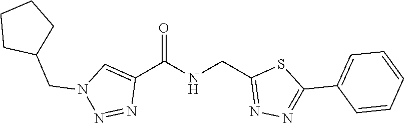 [SMILES: O=C(NCc1nnc(-c2ccccc2)s1)c1cn(CC2CCCC2)nn1] | 1-(cyclopentylmethyl)-N-((5-phenyl-1,3,4- thiadiazol-2-yl)methyl)-1H-1,2,3-triazole-4- carboxamide |
| 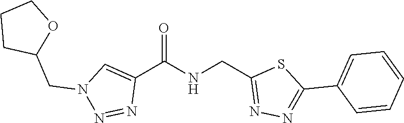 [SMILES: O=C(NCc1nnc(-c2ccccc2)s1)c1cn(CC2CCCO2)nn1] | N-((5-phenyl-1,3,4-thiadiazol-2-yl)methyl)-1- ((tetrahydrofuran-2-yl)methyl)-1H-1,2,3-triazole-4- carboxamide |
| 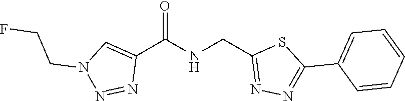 [SMILES: O=C(NCc1nnc(-c2ccccc2)s1)c1cn(CCF)nn1] | 1-(2-fluoroethyl)-N-((5-phenyl-1,3,4-thiadiazol-2- yl)methyl)-1H-1,2,3-triazole-4-carboxamide |
| 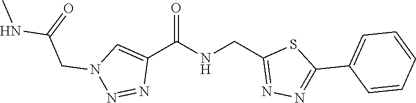 [SMILES: CNC(=O)Cn1cc(C(=O)NCc2nnc(-c3ccccc3)s2)nn1] | 1-(2-(methylamino)-2-oxoethyl)-N-((5-phenyl- 1,3,4-thiadiazol-2-yl)methyl)-1H-1,2,3-triazole-4- carboxamide |
| 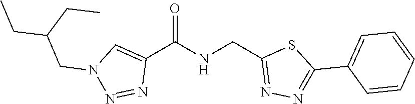 [SMILES: CCC(CC)Cn1cc(C(=O)NCc2nnc(-c3ccccc3)s2)nn1] | 1-(2-ethylbutyl)-N-((5-phenyl-1,3,4-thiadiazol-2- yl)methyl)-1H-1,2,3-triazole-4-carboxamide |
| 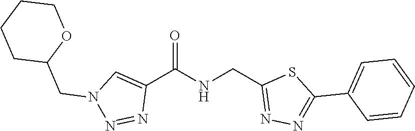 [SMILES: O=C(NCc1nnc(-c2ccccc2)s1)c1cn(CC2CCCCO2)nn1] | N-((5-phenyl-1,3,4-thiadiazol-2-yl)methyl)-1- ((tetrahydro-2H-pyran-2-yl)methyl)-1H-1,2,3- triazole-4-carboxamide |
| 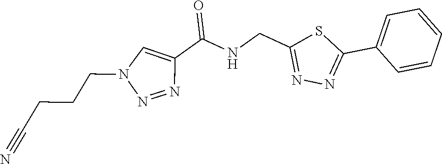 [SMILES: N#CCCCn1cc(C(=O)NCc2nnc(-c3ccccc3)s2)nn1] | 1-(3-cyanopropyl)-N-((5-phenyl-1,3,4-thiadiazol-2- yl)methyl)-1H-1,2,3-triazole-4-carboxamide |
| 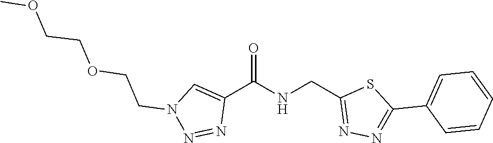 [SMILES: COCCOCCn1cc(C(=O)NCc2nnc(-c3ccccc3)s2)nn1] | 1-(2-(2-methoxyethoxy)ethyl)-N-((5-phenyl-1,3,4- thiadiazol-2-yl)methyl)-1H-1,2,3-triazole-4- carboxamide |
| 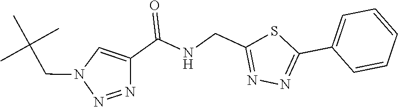 [SMILES: CC(C)(C)Cn1cc(C(=O)NCc2nnc(-c3ccccc3)s2)nn1] | 1-neopentyl-N-((5-phenyl-1,3,4-thiadiazol-2- yl)methyl)-1H-1,2,3-triazole-4-carboxamide |
| 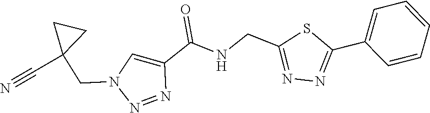 [SMILES: N#CC1(Cn2cc(C(=O)NCc3nnc(-c4ccccc4)s3)nn2)CC1] | 1-((1-cyanocyclopropyl)methyl)-N-((5-phenyl- 1,3,4-thiadiazol-2-yl)methyl)-1H-1,2,3-triazole-4- carboxamide |
| 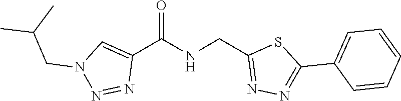 [SMILES: CC(C)Cn1cc(C(=O)NCc2nnc(-c3ccccc3)s2)nn1] | 1-isobutyl-N-((5-phenyl-1,3,4-thiadiazol-2- yl)methyl)-1H-1,2,3-triazole-4-carboxamide |
| 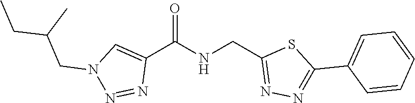 [SMILES: CCC(C)Cn1cc(C(=O)NCc2nnc(-c3ccccc3)s2)nn1] | 1-(2-methylbutyl)-N-((5-phenyl-1,3,4-thiadiazol-2- yl)methyl)-1H-1,2,3-triazole-4-carboxamide |
| 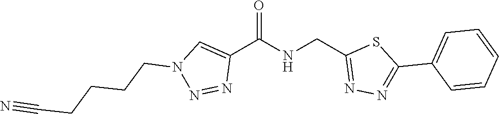 [SMILES: N#CCCCCn1cc(C(=O)NCc2nnc(-c3ccccc3)s2)nn1] | 1-(4-cyanobutyl)-N-((5-phenyl-1,3,4-thiadiazol-2- yl)methyl)-1H-1,2,3-triazole-4-carboxamide |
| 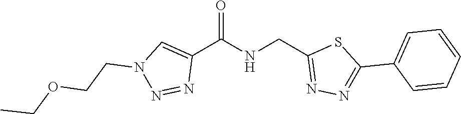 [SMILES: CCOCCn1cc(C(=O)NCc2nnc(-c3ccccc3)s2)nn1] | 1-(2-ethoxyethyl)-N-((5-phenyl-1,3,4-thiadiazol-2- yl)methyl)-1H-1,2,3-triazole-4-carboxamide |
| 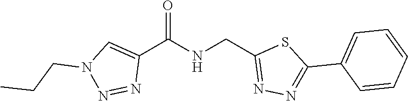 [SMILES: CCCn1cc(C(=O)NCc2nnc(-c3ccccc3)s2)nn1] | N-((5-phenyl-1,3,4-thiadiazol-2-yl)methyl)-1- propyl-1H-1,2,3-triazole-4-carboxamide |
| 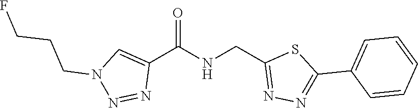 [SMILES: O=C(NCc1nnc(-c2ccccc2)s1)c1cn(CCCF)nn1] | 1-(3-fluoropropyl)-N-((5-phenyl-1,3,4-thiadiazol-2- yl)methyl)-1H-1,2,3-triazole-4-carboxamide |
| 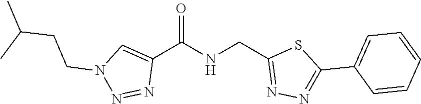 [SMILES: CC(C)CCn1cc(C(=O)NCc2nnc(-c3ccccc3)s2)nn1] | 1-isopentyl-N-((5-phenyl-1,3,4-thiadiazol-2- yl)methyl)-1H-1,2,3-triazole-4-carboxamide |
| 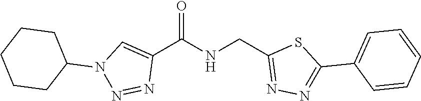 [SMILES: O=C(NCc1nnc(-c2ccccc2)s1)c1cn(C2CCCCC2)nn1] | 1-cyclohexyl-N-((5-phenyl-1,3,4-thiadiazol-2- yl)methyl)-1H-1,2,3-triazole-4-carboxamide |
| 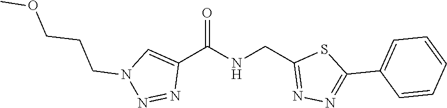 [SMILES: COCCCn1cc(C(=O)NCc2nnc(-c3ccccc3)s2)nn1] | 1-(3-methoxypropyl)-N-((5-phenyl-1,3,4- thiadiazol-2-yl)methyl)-1H-1,2,3-triazole-4- carboxamide |
| 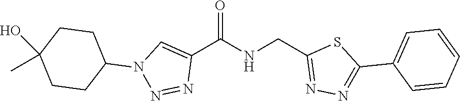 [SMILES: CC1(O)CCC(n2cc(C(=O)NCc3nnc(-c4ccccc4)s3)nn2)CC1] | 1-(4-hydroxy-4-methylcyclohexyl)-N-((5-phenyl- 1,3,4-thiadiazol-2-yl)methyl)-1H-1,2,3-triazole-4- carboxamide |
| 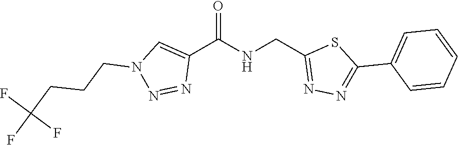 [SMILES: O=C(NCc1nnc(-c2ccccc2)s1)c1cn(CCCC(F)(F)F)nn1] | N-((5-phenyl-1,3,4-thiadiazol-2-yl)methyl)-1- (4,4,4-trifluorobutyl)-1H-1,2,3-triazole-4- carboxamide |
| 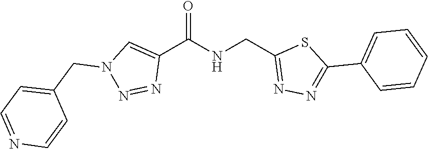 [SMILES: O=C(NCc1nnc(-c2ccccc2)s1)c1cn(Cc2ccncc2)nn1] | N-((5-phenyl-1,3,4-thiadiazol-2-yl)methyl)-1- (pyridin-4-ylmethyl)-1H-1,2,3-triazole-4- carboxamide |
| 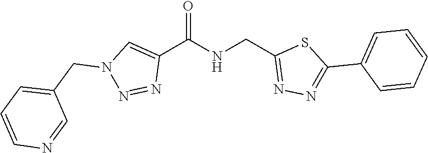 [SMILES: O=C(NCc1nnc(-c2ccccc2)s1)c1cn(Cc2cccnc2)nn1] | N-((5-phenyl-1,3,4-thiadiazol-2-yl)methyl)-1- (pyridin-3-ylmethyl)-1H-1,2,3-triazole-4- carboxamide |
| 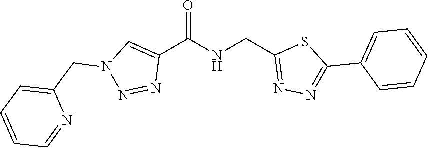 [SMILES: O=C(NCc1nnc(-c2ccccc2)s1)c1cn(Cc2ccccn2)nn1] | N-((5-phenyl-1,3,4-thiadiazol-2-yl)methyl)-1- (pyridin-2-ylmethyl)-1H-1,2,3-triazole-4- carboxamide |
| 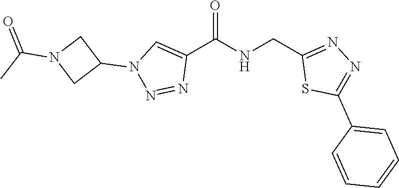 [SMILES: CC(=O)N1CC(n2cc(C(=O)NCc3nnc(-c4ccccc4)s3)nn2)C1] | 1-(1-acetylazetidin-3-yl)-N-((5-phenyl-1,3,4- thiadiazol-2-yl)methyl)-1H-1,2,3-triazole-4- carboxamide |
| 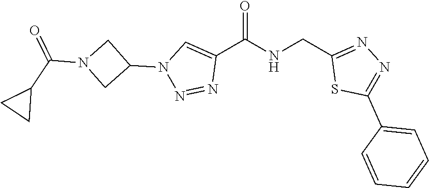 [SMILES: O=C(NCc1nnc(-c2ccccc2)s1)c1cn(C2CN(C(=O)C3CC3)C2)nn1] | 1-(1-(cyclopropanecarbonyl)azetidin-3-yl)-N-((5- phenyl-1,3,4-thiadiazol-2-yl)methyl)-1H-1,2,3- triazole-4-carboxamide |
| 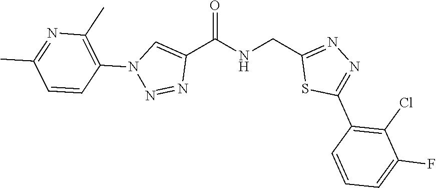 [SMILES: Cc1ccc(-n2cc(C(=O)NCc3nnc(-c4cccc(F)c4Cl)s3)nn2)c(C)n1] | N-((5-(3-chloro-2-fluorophenyl)-1,3,4-thiadiazol-2- yl)methyl)-1-(2,6-dimethylpyridin-3-yl)-1H-1,2,3- triazole-4-carboxamide |
| 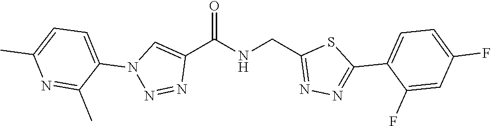 [SMILES: Cc1ccc(-n2cc(C(=O)NCc3nnc(-c4ccc(F)cc4F)s3)nn2)c(C)n1] | N-((5-(2,4-difluorophenyl)-1,3,4-thiadiazol-2- yl)methyl)-1-(2,6-dimethylpyridin-3-yl)-1H-1,2,3- triazole-4-carboxamide |
| 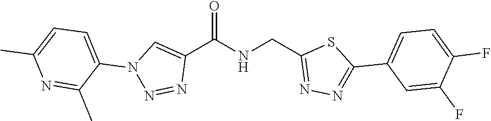 [SMILES: Cc1ccc(-n2cc(C(=O)NCc3nnc(-c4ccc(F)c(F)c4)s3)nn2)c(C)n1] | N-((5-(3,4-difluorophenyl)-1,3,4-thiadiazol-2- yl)methyl)-1-(2,6-dimethylpyridin-3-yl)-1H-1,2,3- triazole-4-carboxamide |
| 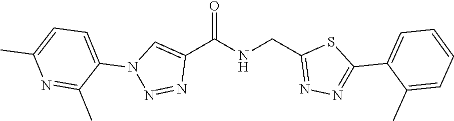 [SMILES: Cc1ccc(-n2cc(C(=O)NCc3nnc(-c4ccccc4C)s3)nn2)c(C)n1] | 1-(2,6-dimethylpyridin-3-yl)-N-((5-(o-tolyl)-1,3,4- thiadiazol-2-yl)methyl)-1H-1,2,3-triazole-4- carboxamide |
| 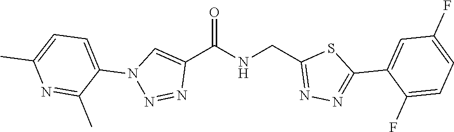 [SMILES: Cc1ccc(-n2cc(C(=O)NCc3nnc(-c4cc(F)ccc4F)s3)nn2)c(C)n1] | N-((5-(2,5-difluorophenyl)-1,3,4-thiadiazol-2- yl)methyl)-1-(2,6-dimethylpyridin-3-yl)-1H-1,2,3- triazole-4-carboxamide |
| 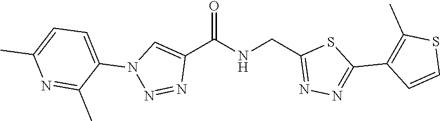 [SMILES: Cc1ccc(-n2cc(C(=O)NCc3nnc(-c4ccsc4C)s3)nn2)c(C)n1] | 1-(2,6-dimethylpyridin-3-yl)-N-((5-(2- methylthiophen-3-yl)-1,3,4-thiadiazol-2- yl)methyl)-1H-1,2,3-triazole-4-carboxamide |
| 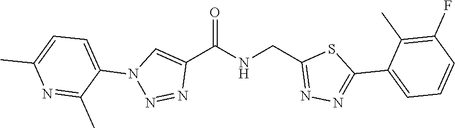 [SMILES: Cc1ccc(-n2cc(C(=O)NCc3nnc(-c4cccc(F)c4C)s3)nn2)c(C)n1] | 1-(2,6-dimethylpyridin-3-yl)-N-((5-(3-fluoro-2- methylphenyl)-1,3,4-thiadiazol-2-yl)methyl)-1H- 1,2,3-triazole-4-carboxamide |
| 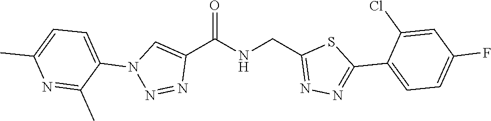 [SMILES: Cc1ccc(-n2cc(C(=O)NCc3nnc(-c4ccc(F)cc4Cl)s3)nn2)c(C)n1] | N-((5-(2-chloro-4-fluorophenyl)-1,3,4-thiadiazol-2- yl)methyl)-1-(2,6-dimethylpyridin-3-yl)-1H-1,2,3- triazole-4-carboxamide |
| 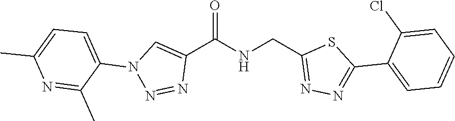 [SMILES: Cc1ccc(-n2cc(C(=O)NCc3nnc(-c4ccccc4Cl)s3)nn2)c(C)n1] | N-((5-(2-chlorophenyl)-1,3,4-thiadiazol-2- yl)methyl)-1-(2,6-dimethylpyridin-3-yl)-1H-1,2,3- triazole-4-carboxamide |
| 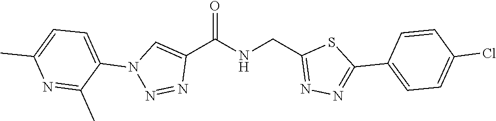 [SMILES: Cc1ccc(-n2cc(C(=O)NCc3nnc(-c4ccc(Cl)cc4)s3)nn2)c(C)n1] | N-((5-(4-chlorophenyl)-1,3,4-thiadiazol-2- yl)methyl)-1-(2,6-dimethylpyridin-3-yl)-1H-1,2,3- triazole-4-carboxamide |
| 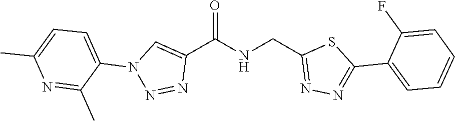 [SMILES: Cc1ccc(-n2cc(C(=O)NCc3nnc(-c4ccccc4F)s3)nn2)c(C)n1] | 1-(2,6-dimethylpyridin-3-yl)-N-((5-(2- fluorophenyl)-1,3,4-thiadiazol-2-yl)methyl)-1H- 1,2,3-triazole-4-carboxamide |
| 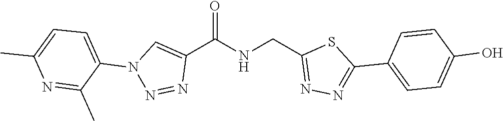 [SMILES: Cc1ccc(-n2cc(C(=O)NCc3nnc(-c4ccc(O)cc4)s3)nn2)c(C)n1] | 1-(2,6-dimethylpyridin-3-yl)-N-((5-(4- hydroxyphenyl)-1,3,4-thiadiazol-2-yl)methyl)-1H- 1,2,3-triazole-4-carboxamide |
| 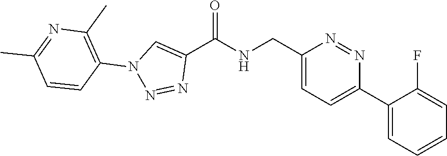 [SMILES: Cc1ccc(-n2cc(C(=O)NCc3ccc(-c4ccccc4F)nn3)nn2)c(C)n1] | Preparation of 1-(2,6-dimethylpyridin-3-yl)-N-((6- (2-fluorophenyl)pyridazin-3-yl)methyl)-1H-1,2,3- triazole-4-carboxamide |
| 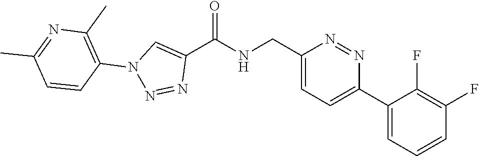 [SMILES: Cc1ccc(-n2cc(C(=O)NCc3ccc(-c4cccc(F)c4F)nn3)nn2)c(C)n1] | N-((6-(2,3-difluorophenyl)pyridazin-3-yl)methyl)- 1-(2,6-dimethylpyridin-3-yl)-1H-1,2,3-triazole-4- carboxamide |
| 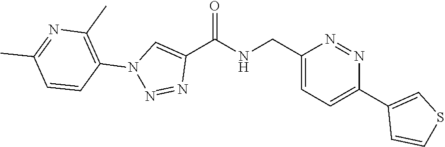 [SMILES: Cc1ccc(-n2cc(C(=O)NCc3ccc(-c4ccsc4)nn3)nn2)c(C)n1] | 1-(2,6-dimethylpyridin-3-yl)-N-((6-(thiophen-3- yl)pyridazin-3-yl)methyl)-1H-1,2,3-triazole-4- carboxamide |
| 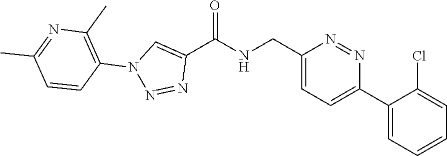 [SMILES: Cc1ccc(-n2cc(C(=O)NCc3ccc(-c4ccccc4Cl)nn3)nn2)c(C)n1] | N-((6-(2-chlorophenyl)pyridazin-3-yl)methyl)-1- (2,6-dimethylpyridin-3-yl)-1H-1,2,3-triazole-4- carboxamide |
| 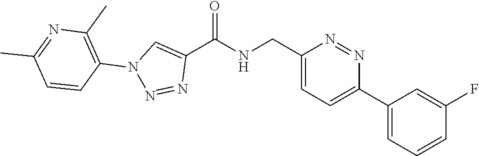 [SMILES: Cc1ccc(-n2cc(C(=O)NCc3ccc(-c4cccc(F)c4)nn3)nn2)c(C)n1] | 1-(2,6-dimethylpyridin-3-yl)-N-((6-(3- fluorophenyl)pyridazin-3-yl)methyl)-1H-1,2,3- triazole-4-carboxamide |
| 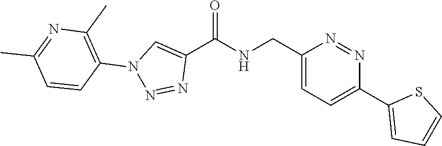 [SMILES: Cc1ccc(-n2cc(C(=O)NCc3ccc(-c4cccs4)nn3)nn2)c(C)n1] | 1-(2,6-dimethylpyridin-3-yl)-N-((6-(thiophen-2- yl)pyridazin-3-yl)methyl)-1H-1,2,3-triazole-4- carboxamide |
| 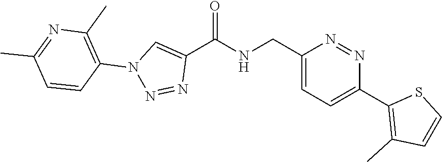 [SMILES: Cc1ccc(-n2cc(C(=O)NCc3ccc(-c4sccc4C)nn3)nn2)c(C)n1] | 1-(2,6-dimethylpyridin-3-yl)-N-((6-(3- methylthiophen-2-yl)pyridazin-3-yl)methyl)-1H- 1,2,3-triazole-4-carboxamide |
| 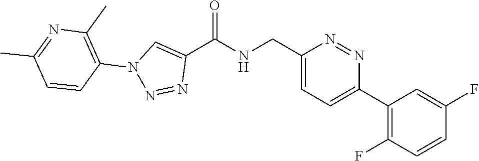 [SMILES: Cc1ccc(-n2cc(C(=O)NCc3ccc(-c4cc(F)ccc4F)nn3)nn2)c(C)n1] | N-((6-(2,5-difluorophenyl)pyridazin-3-yl)methyl)- 1-(2,6-dimethylpyridin-3-yl)-1H-1,2,3-triazole-4- carboxamide |
| 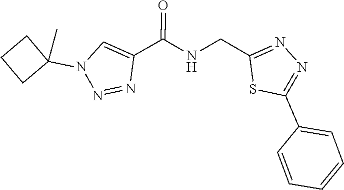 [SMILES: CC1(n2cc(C(=O)NCc3nnc(-c4ccccc4)s3)nn2)CCC1] | 1-(1-methylcyclobutyl)-N-((5-phenyl-1,3,4- thiadiazol-2-yl)methyl)-1H-1,2,3-triazole-4- carboxamide |
| 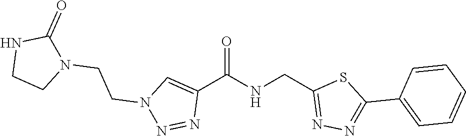 [SMILES: O=C(NCc1nnc(-c2ccccc2)s1)c1cn(CCN2CCNC2=O)nn1] | 1-(2-(2-oxoimidazolidin-1-yl)ethyl)-N-((5-phenyl- 1,3,4-thiadiazol-2-yl)methyl)-1H-1,2,3-triazole-4- carboxamide |
| 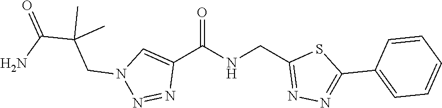 [SMILES: CC(C)(Cn1cc(C(=O)NCc2nnc(-c3ccccc3)s2)nn1)C(N)=O] | 1-(3-amino-2,2-dimethyl-3-oxo-propyl)-N-[(5- phenyl-1,3,4-thiadiazol-2-yl)methyl]triazole-4- carboxamide |
| 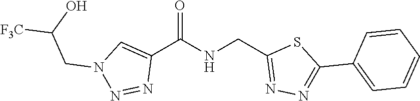 [SMILES: O=C(NCc1nnc(-c2ccccc2)s1)c1cn(CC(O)C(F)(F)F)nn1] | N-((5-phenyl-1,3,4-thiadiazol-2-yl)methyl)-1- (3,3,3-trifluoro-2-hydroxypropyl)-1H-1,2,3- triazole-4-carboxamide |
| 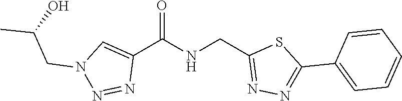 [SMILES: C[C@H](O)Cn1cc(C(=O)NCc2nnc(-c3ccccc3)s2)nn1] | (S)-1-(2-hydroxypropyl)-N-((5-phenyl-1,3,4- thiadiazol-2-yl)methyl)-1H-1,2,3-triazole-4- carboxamide |
| 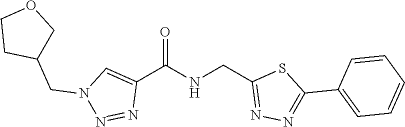 [SMILES: O=C(NCc1nnc(-c2ccccc2)s1)c1cn(CC2CCOC2)nn1] | N-((5-phenyl-1,3,4-thiadiazol-2-yl)methyl)-1- ((tetrahydrofuran-3-yl)methyl)-1H-1,2,3-triazole-4- carboxamide |
| 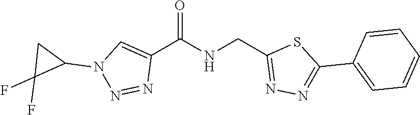 [SMILES: O=C(NCc1nnc(-c2ccccc2)s1)c1cn(C2CC2(F)F)nn1] | 1-(2,2-difluorocyclopropyl)-N-((5-phenyl-1,3,4- thiadiazol-2-yl)methyl)-1H-1,2,3-triazole-4- carboxamide |
| 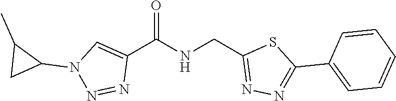 [SMILES: CC1CC1n1cc(C(=O)NCc2nnc(-c3ccccc3)s2)nn1] | Cis-1-(2-methylcyclopropyl)-N-((5-phenyl-1,3,4- thiadiazol-2-yl)methyl)-1H-1,2,3-triazole-4- carboxamide |
| 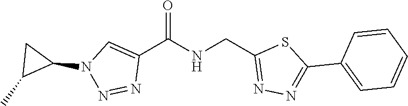 [SMILES: C[C@@H]1C[C@H]1n1cc(C(=O)NCc2nnc(-c3ccccc3)s2)nn1] | 1-((1R,2R)-2-methylcyclopropyl)-N-((5-phenyl- 1,3,4-thiadiazol-2-yl)methyl)-1H-1,2,3-triazole-4- carboxamide |
| 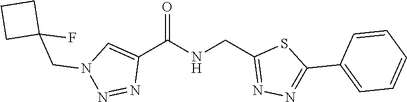 [SMILES: O=C(NCc1nnc(-c2ccccc2)s1)c1cn(CC2(F)CCC2)nn1] | 1-((1-fluorocyclobutyl)methyl)-N-((5-phenyl-1,3,4- thiadiazol-2-yl)methyl)-1H-1,2,3-triazole-4- carboxamide |
| 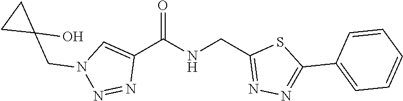 [SMILES: O=C(NCc1nnc(-c2ccccc2)s1)c1cn(CC2(O)CC2)nn1] | 1-((1-hydroxycyclopropyl)methyl)-N-((5-phenyl- 1,3,4-thiadiazol-2-yl)methyl)-1H-1,2,3-triazole-4- carboxamide |
| 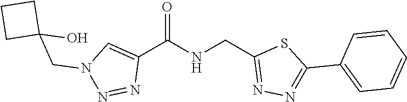 [SMILES: O=C(NCc1nnc(-c2ccccc2)s1)c1cn(CC2(O)CCC2)nn1] | 1-((1-hydroxycyclobutyl)methyl)-N-((5-phenyl- 1,3,4-thiadiazol-2-yl)methyl)-1H-1,2,3-triazole-4- carboxamide |
| 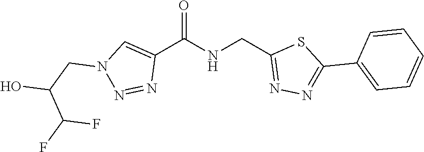 [SMILES: O=C(NCc1nnc(-c2ccccc2)s1)c1cn(CC(O)C(F)F)nn1] | 1-(3,3-difluoro-2-hydroxypropyl)-N-((5-phenyl- 1,3,4-thiadiazol-2-yl)methyl)-1H-1,2,3-triazole-4- carboxamide |
| 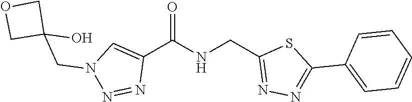 [SMILES: O=C(NCc1nnc(-c2ccccc2)s1)c1cn(CC2(O)COC2)nn1] | 1-((3-hydroxyoxetan-3-yl)methyl)-N-((5-phenyl- 1,3,4-thiadiazol-2-yl)methyl)-1H-1,2,3-triazole-4- carboxamide |
| 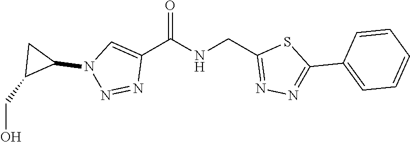 [SMILES: O=C(NCc1nnc(-c2ccccc2)s1)c1cn([C@@H]2C[C@H]2CO)nn1] | Trans-1-(2-(hydroxymethyl)cyclopropyl)-N-((5- phenyl-1,3,4-thiadiazol-2-yl)methyl)-1H-1,2,3- triazole-4-carboxamide |
| 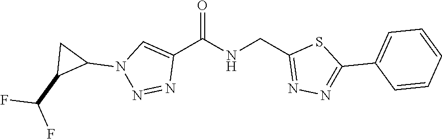 | Trans-1-(-2-(difluoromethyl)cyclopropyl]-N-[(5- phenyl-1,3,4-thiadiazol-2-yl)methyl]-1H-1,2,3- triazole-4-carboxamide |
| 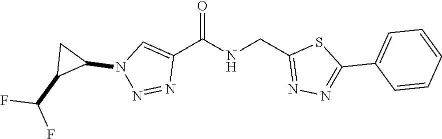 | cis-1-(-2-(difluoromethyl)cyclopropyl]-N-[(5- phenyl-1,3,4-thiadiazol-2-yl)methyl]-1H-1,2,3- triazole-4-carboxamide |
| 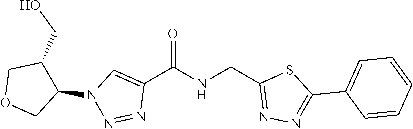 [SMILES: O=C(NCc1nnc(-c2ccccc2)s1)c1cn([C@H]2COC[C@@H]2CO)nn1] | 1-[(3S,4R)-4-(hydroxymethyl)oxolan-3-yl]-N-[(5- phenyl-1,3,4-thiadiazol-2-yl)methyl]-1H-1,2,3- triazole-4-carboxamide |
| 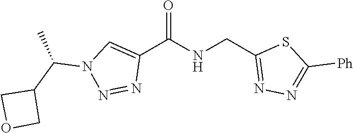 [SMILES: C[C@@H](C1COC1)n1cc(C(=O)NCc2nnc(-c3ccccc3)s2)nn1] | (S)-1-(1-(oxetan-3-yl)ethyl)-N-((5-phenyl-1,3,4- thiadiazol-2-yl)methyl)-1H-1,2,3-triazole-4- carboxamide |
| 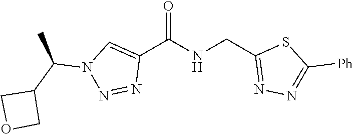 [SMILES: C[C@H](C1COC1)n1cc(C(=O)NCc2nnc(-c3ccccc3)s2)nn1] | (R)-1-(1-(oxetan-3-yl)ethyl)-N-((5-phenyl-1,3,4- thiadiazol-2-yl)methyl)-1H-1,2,3-triazole-4- carboxamide |
| 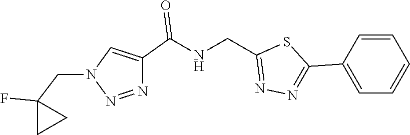 [SMILES: O=C(NCc1nnc(-c2ccccc2)s1)c1cn(CC2(F)CC2)nn1] | 1-((1-fluorocyclopropyl) methyl)-N-((5-phenyl-1,3,4-thiadiazol-2- yl)methyl)-1H-1,2,3-triazole-4-carboxamide |
| 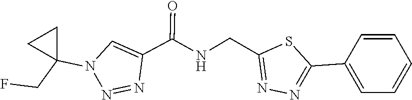 [SMILES: O=C(NCc1nnc(-c2ccccc2)s1)c1cn(C2(CF)CC2)nn1] | 1-(1-(fluoromethyl)cyclopropyl)-N-((5-phenyl- 1,3,4-thiadiazol-2-yl)methyl)-1H-1,2,3-triazole-4- carboxamide |
| 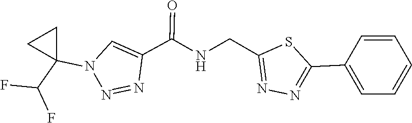 [SMILES: O=C(NCc1nnc(-c2ccccc2)s1)c1cn(C2(C(F)F)CC2)nn1] | 1-(1-(difluoromethyl)cyclopropyl)-N-((5-phenyl- 1,3,4-thiadiazol-2-yl)methyl)-1H-1,2,3-triazole-4- carboxamide |
| 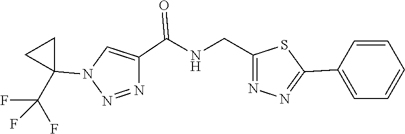 [SMILES: O=C(NCc1nnc(-c2ccccc2)s1)c1cn(C2(C(F)(F)F)CC2)nn1] | N-((5-phenyl-1,3,4-thiadiazol-2-yl)methyl)-1-(1- (trifluoromethyl)cyclopropyl)-1H-1,2,3-triazole-4- carboxamide |
| 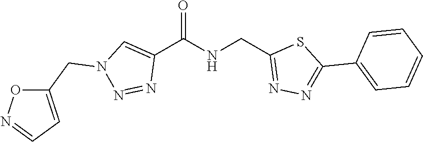 [SMILES: O=C(NCc1nnc(-c2ccccc2)s1)c1cn(Cc2ccno2)nn1] | 1-(isoxazol-5-ylmethyl)-N-((5-phenyl-1,3,4- thiadiazol-2-yl)methyl)-1H-1,2,3-triazole-4- carboxamide |
| 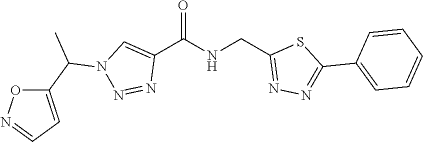 [SMILES: CC(c1ccno1)n1cc(C(=O)NCc2nnc(-c3ccccc3)s2)nn1] | 1-(1-(isoxazol-5-yl) ethyl)-N-((5-phenyl-1,3,4-thiadiazol-2-yl)methyl)- 1H-1,2,3-triazole-4-carboxamide |
| 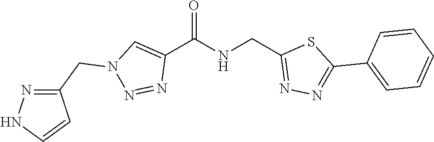 [SMILES: O=C(NCc1nnc(-c2ccccc2)s1)c1cn(Cc2cc[nH]n2)nn1] | 1-((1H-pyrazol-3-yl) methyl)-N-((5-phenyl-1,3,4-thiadiazol-2- yl)methyl)-1H-1,2,3-triazole-4-carboxamide |
| 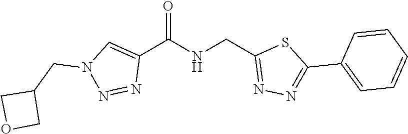 [SMILES: O=C(NCc1nnc(-c2ccccc2)s1)c1cn(CC2COC2)nn1] | 1-(oxetan-3-ylmethyl)-N-((5-phenyl-1,3,4- thiadiazol-2-yl)methyl)-1H-1,2,3-triazole-4- carboxamide |
| 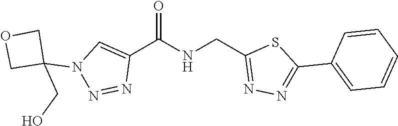 [SMILES: O=C(NCc1nnc(-c2ccccc2)s1)c1cn(C2(CO)COC2)nn1] | 1-(3-(hydroxymethyl)oxetan-3-yl)-N-((5-phenyl- 1,3,4-thiadiazol-2-yl)methyl)-1H- 1,2,3-triazole-4-carboxamide |
| 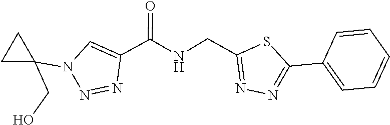 [SMILES: O=C(NCc1nnc(-c2ccccc2)s1)c1cn(C2(CO)CC2)nn1] | 1-(1-(hydroxymethyl)cyclopropyl)-N-((5-phenyl- 1,3,4-thiadiazol-2-yl)methyl)-1H- 1,2,3-triazole-4-carboxamide |
| 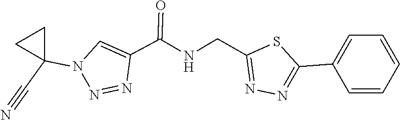 [SMILES: N#CC1(n2cc(C(=O)NCc3nnc(-c4ccccc4)s3)nn2)CC1] | 1-(1-cyanocyclopropyl)-N-((5-phenyl-1,3,4- thiadiazol-2-yl)methyl)-1H-1,2,3-triazole-4- carboxamide |
| 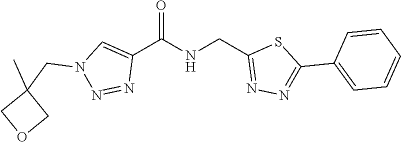 [SMILES: CC1(Cn2cc(C(=O)NCc3nnc(-c4ccccc4)s3)nn2)COC1] | 1-((3-methyloxetan-3-yl)methyl)-N-((5-phenyl- 1,3,4-thiadiazol-2-yl)methyl)-1H-1,2,3-triazole-4- carboxamide |
| 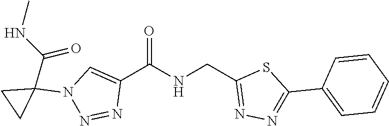 [SMILES: CNC(=O)C1(n2cc(C(=O)NCc3nnc(-c4ccccc4)s3)nn2)CC1] | 1-(1-(methylcarbamoyl)cyclopropyl)-N-((5-phenyl- 1,3,4-thiadiazol-2-yl)methyl)-1H- 1,2,3-triazole-4-carboxamide |
| 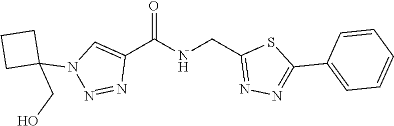 [SMILES: O=C(NCc1nnc(-c2ccccc2)s1)c1cn(C2(CO)CCC2)nn1] | 1-(1-(hydroxymethyl)cyclobutyl)-N-((5-phenyl- 1,3,4-thiadiazol-2-yl)methyl)-1H-1,2,3-triazole-4- carboxamide |
| 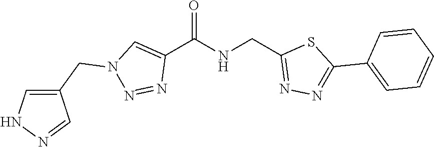 [SMILES: O=C(NCc1nnc(-c2ccccc2)s1)c1cn(Cc2cn[nH]c2)nn1] | 1-((1H-pyrazol-4-yl) methyl)-N-((5-phenyl-1,3,4-thiadiazol-2- yl)methyl)-1H-1,2,3-triazole-4-carboxamide |
| 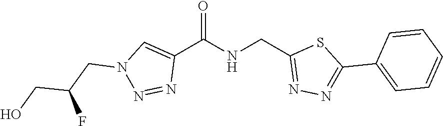 [SMILES: O=C(NCc1nnc(-c2ccccc2)s1)c1cn(C[C@@H](F)CO)nn1] | (R)-1-(2-fluoro-3-hydroxypropyl)-N-((5-phenyl- 1,3,4-thiadiazol-2-yl)methyl)-1H-1,2,3-triazole-4- carboxamide |
| 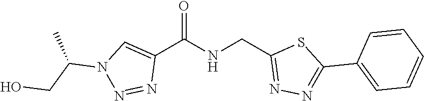 [SMILES: C[C@@H](CO)n1cc(C(=O)NCc2nnc(-c3ccccc3)s2)nn1] | (S)-1-(1-hydroxypropan-2-yl)-N-((5-phenyl-1,3,4- thiadiazol-2-yl)methyl)-1H-1,2,3-triazole-4- carboxamide |
| 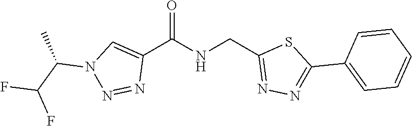 [SMILES: C[C@@H](C(F)F)n1cc(C(=O)NCc2nnc(-c3ccccc3)s2)nn1] | (S)-1-(1,1-difluoropropan-2-yl)-N-((5-phenyl- 1,3,4-thiadiazol-2-yl)methyl)-1H-1,2,3-triazole-4- carboxamide |
| 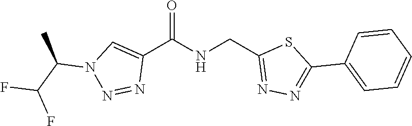 [SMILES: C[C@H](C(F)F)n1cc(C(=O)NCc2nnc(-c3ccccc3)s2)nn1] | (R)-1-(1,1-difluoropropan-2-yl)-N-((5-phenyl- 1,3,4-thiadiazol-2-yl)methyl)-1H-1,2,3-triazole-4- carboxamide |
| 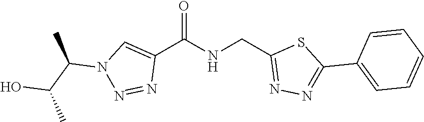 [SMILES: C[C@H](O)[C@@H](C)n1cc(C(=O)NCc2nnc(-c3ccccc3)s2)nn1] | 1-((2R,3S)-3-hydroxybutan-2-yl)-N-((5-phenyl- 1,3,4-thiadiazol-2-yl)methyl)-1H-1,2,3-triazole-4- carboxamide |
| 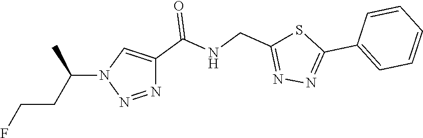 [SMILES: C[C@H](CCF)n1cc(C(=O)NCc2nnc(-c3ccccc3)s2)nn1] | (R)-1-(4-fluorobutan-2-yl)-N-((5-phenyl-1,3,4- thiadiazol-2-yl)methyl)-1H-1,2,3-triazole-4- carboxamide |
| 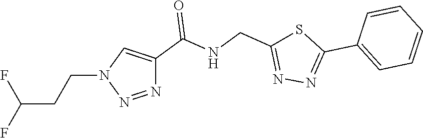 [SMILES: O=C(NCc1nnc(-c2ccccc2)s1)c1cn(CCC(F)F)nn1] | 1-(3,3-difluoropropyl)-N-((5-phenyl-1,3,4- thiadiazol-2-yl)methyl)-1H-1,2,3-triazole-4- carboxamide |
| 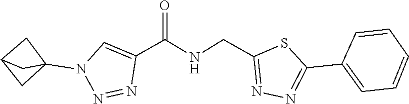 [SMILES: O=C(NCc1nnc(-c2ccccc2)s1)c1cn(C23CC(C2)C3)nn1] | 1-(bicyclo[1.1.1]pentan-1-yl)-N-((5-phenyl-1,3,4- thiadiazol-2-yl)methyl)-1H-1,2,3-triazole-4- carboxamide |
| 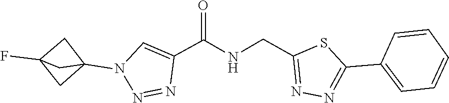 [SMILES: O=C(NCc1nnc(-c2ccccc2)s1)c1cn(C23CC(F)(C2)C3)nn1] | 1-(3-fluorobicyclo[1.1.1]pentan-1-yl)-N-((5- phenyl-1,3,4-thiadiazol-2-yl)methyl)-1H-1,2,3- triazole-4-carboxamide |
| 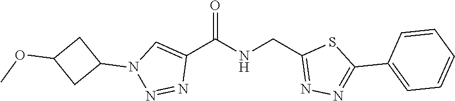 [SMILES: COC1CC(n2cc(C(=O)NCc3nnc(-c4ccccc4)s3)nn2)C1] | 1-(3-methoxycyclobutyl)-N-((5-phenyl-1,3,4- thiadiazol-2-yl)methyl)-1H-1,2,3-triazole-4- carboxamide |
| 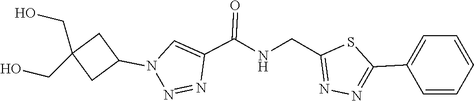 [SMILES: O=C(NCc1nnc(-c2ccccc2)s1)c1cn(C2CC(CO)(CO)C2)nn1] | Preparation of 1-(3,3- bis(hydroxymethyl)cyclobutyl)-N-((5-phenyl-1,3,4- thiadiazol-2-yl)methyl)-1H-1,2,3-triazole-4- carboxamide |
| 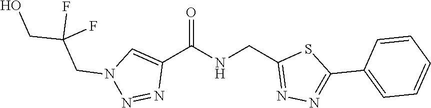 [SMILES: O=C(NCc1nnc(-c2ccccc2)s1)c1cn(CC(F)(F)CO)nn1] | 1-(2,2-difluoro-3-hydroxypropyl)-N-((5-phenyl- 1,3,4-thiadiazol-2-yl)methyl)-1H-1,2,3-triazole-4- carboxamide |
| 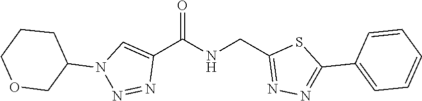 [SMILES: O=C(NCc1nnc(-c2ccccc2)s1)c1cn(C2CCCOC2)nn1] | N-((5-phenyl-1,3,4-thiadiazol-2-yl)methyl)-1- (tetrahydro-2H-pyran-3-yl)-1H-1,2,3-triazole-4- carboxamide |
| 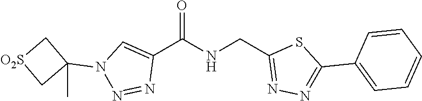 [SMILES: CC1(n2cc(C(=O)NCc3nnc(-c4ccccc4)s3)nn2)CS(=O)(=O)C1] | 1-(3-methyl-1,1-dioxidothietan-3-yl)-N-((5-phenyl- 1,3,4-thiadiazol-2-yl)methyl)-1H-1,2,3-triazole-4- carboxamide |
| 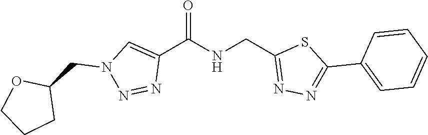 [SMILES: O=C(NCc1nnc(-c2ccccc2)s1)c1cn(C[C@H]2CCCO2)nn1] | (R)-N-((5-phenyl-1,3,4-thiadiazol-2-yl)methyl)-1- ((tetrahydrofuran-2-yl)methyl)-1H-1,2,3-triazole-4- carboxamide |
| 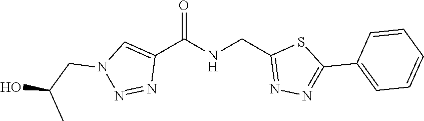 [SMILES: C[C@@H](O)Cn1cc(C(=O)NCc2nnc(-c3ccccc3)s2)nn1] | (R)-1-(2-hydroxypropyl)-N-((5-phenyl-1,3,4- thiadiazol-2-yl)methyl)-1H-1,2,3-triazole-4- carboxamide |
| 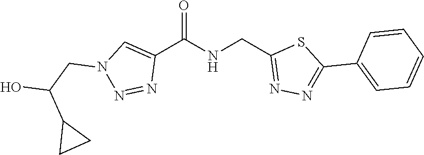 [SMILES: O=C(NCc1nnc(-c2ccccc2)s1)c1cn(CC(O)C2CC2)nn1] | 1-(2-cyclopropyl-2-hydroxyethyl)-N-((5-phenyl- 1,3,4-thiadiazol-2-yl)methyl)-1H-1,2,3-triazole-4- carboxamide |
| 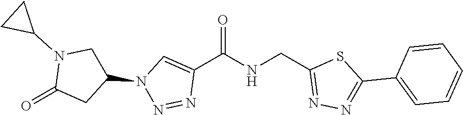 | (R)-1-(1-cyclopropyl-5-oxopyrrolidin-3-yl)-N-((5- phenyl-1,3,4-thiadiazol-2-yl)methyl)-1H-1,2,3- triazole-4-carboxamide |
| 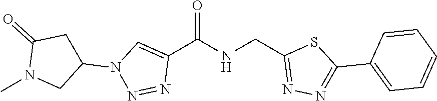 [SMILES: CN1CC(n2cc(C(=O)NCc3nnc(-c4ccccc4)s3)nn2)CC1=O] | 1-(1-methyl-5-oxopyrrolidin-3-yl)-N-((5-phenyl- 1,3,4-thiadiazol-2-yl)methyl)-1H-1,2,3-triazole-4- carboxamide |
| 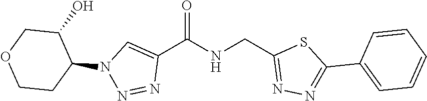 [SMILES: O=C(NCc1nnc(-c2ccccc2)s1)c1cn([C@H]2CCOC[C@@H]2O)nn1] | 1-((3R,4S)-3-hydroxytetrahydro-2H-pyran-4-yl)-N- ((5-phenyl-1,3,4-thiadiazol-2-yl)methyl)-1H-1,2,3- triazole-4-carboxamide |
| 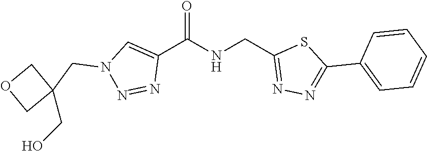 [SMILES: O=C(NCc1nnc(-c2ccccc2)s1)c1cn(CC2(CO)COC2)nn1] | 1-((3-(hydroxymethyl)oxetan-3-yl)methyl)-N-((5- phenyl-1,3,4-thiadiazol-2-yl)methyl)-1H-1,2,3- triazole-4-carboxamide |
| 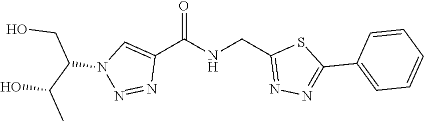 | 1-((2S,3S)-1,3-dihydroxybutan-2-yl)-N-((5-phenyl- 1,3,4-thiadiazol-2-yl)methyl)-1H-1,2,3-triazole-4- carboxamide |
| 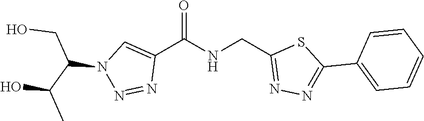 | 1-((2S,3R)-1,3-dihydroxybutan-2-yl)-N-((5-phenyl- 1,3,4-thiadiazol-2-yl)methyl)-1H-1,2,3-triazole-4- carboxamide |
| 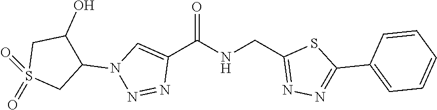 [SMILES: O=C(NCc1nnc(-c2ccccc2)s1)c1cn(C2CS(=O)(=O)CC2O)nn1] | 1-(4-hydroxy-1,1-dioxidotetrahydrothiophen-3-yl)- N-((5-phenyl-1,3,4-thiadiazol-2-yl)methyl)-1H- 1,2,3-triazole-4-carboxamide |
| 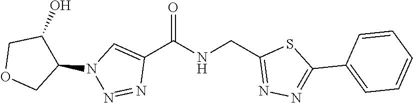 [SMILES: O=C(NCc1nnc(-c2ccccc2)s1)c1cn([C@H]2COC[C@@H]2O)nn1] | 1-((3S,4R)-4-hydroxytetrahydrofuran-3-yl)-N-((5- phenyl-1,3,4-thiadiazol-2-yl)methyl)-1H-1,2,3- triazole-4-carboxamide |
| 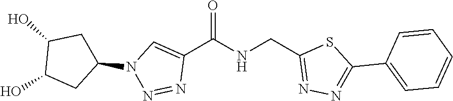 [SMILES: O=C(NCc1nnc(-c2ccccc2)s1)c1cn([C@@H]2C[C@@H](O)[C@@H](O)C2)nn1] | 1-((1s,3R,4S)-3,4-dihydroxycyclopentyl)-N-((5- phenyl-1,3,4-thiadiazol-2-yl)methyl)-1H-1,2,3- triazole-4-carboxamide |
| 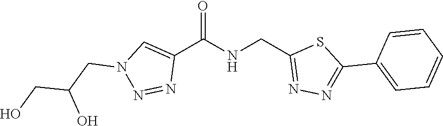 [SMILES: O=C(NCc1nnc(-c2ccccc2)s1)c1cn(CC(O)CO)nn1] | 1-(2,3-dihydroxypropyl)-N-((5-phenyl-1,3,4- thiadiazol-2-yl)methyl)-1H-1,2,3-triazole-4- carboxamide |
| 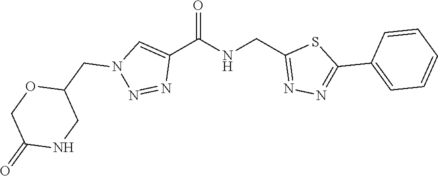 [SMILES: O=C1COC(Cn2cc(C(=O)NCc3nnc(-c4ccccc4)s3)nn2)CN1] | 1-((5-oxomorpholin-2-yl)methyl)-N-((5-phenyl- 1,3,4-thiadiazol-2-yl)methyl)-1H-1,2,3-triazole-4- carboxamide |
| 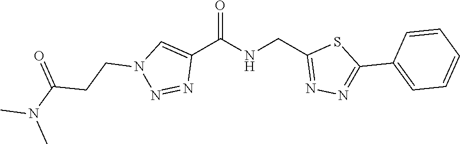 [SMILES: CN(C)C(=O)CCn1cc(C(=O)NCc2nnc(-c3ccccc3)s2)nn1] | 1-(3-(dimethylamino)-3-oxopropyl)-N-((5-phenyl- 1,3,4-thiadiazol-2-yl)methyl)-1H-1,2,3-triazole-4- carboxamide |
| 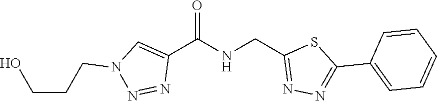 [SMILES: O=C(NCc1nnc(-c2ccccc2)s1)c1cn(CCCO)nn1] | 1-(3-hydroxypropyl)-N-((5-phenyl-1,3,4-thiadiazol- 2-yl)methyl)-1H-1,2,3-triazole-4-carboxamide |
| 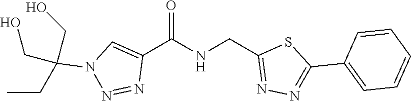 [SMILES: CCC(CO)(CO)n1cc(C(=O)NCc2nnc(-c3ccccc3)s2)nn1] | 1-(1-hydroxy-2-(hydroxymethyl)butan-2-yl)-N-((5- phenyl-1,3,4-thiadiazol-2-yl)methyl)-1H-1,2,3- triazole-4-carboxamide |
| 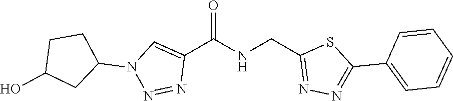 [SMILES: O=C(NCc1nnc(-c2ccccc2)s1)c1cn(C2CCC(O)C2)nn1] | 1-(3-hydroxycyclopentyl)-N-((5-phenyl-1,3,4- thiadiazol-2-yl)methyl)-1H-1,2,3-triazole-4- carboxamide |
| 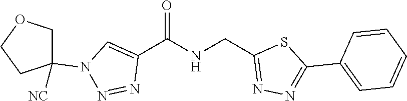 [SMILES: N#CC1(n2cc(C(=O)NCc3nnc(-c4ccccc4)s3)nn2)CCOC1] | 1-(3-cyanotetrahydrofuran-3-yl)-N-((5-phenyl- 1,3,4-thiadiazol-2-yl)methyl)-1H-1,2,3-triazole-4- carboxamide |
| 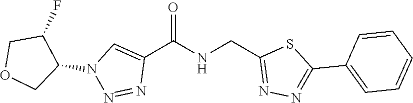 [SMILES: O=C(NCc1nnc(-c2ccccc2)s1)c1cn([C@@H]2COC[C@@H]2F)nn1] | 1-((3R,4R)-4-fluorotetrahydrofuran-3-yl)-N-((5- phenyl-1,3,4-thiadiazol-2-yl)methyl)-1H-1,2,3- triazole-4-carboxamide |
| 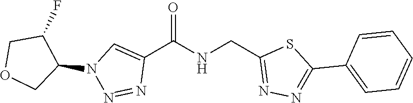 [SMILES: O=C(NCc1nnc(-c2ccccc2)s1)c1cn([C@H]2COC[C@@H]2F)nn1] | 1-((3S,4R)-4-fluorotetrahydrofuran-3-yl)-N-((5- phenyl-1,3,4-thiadiazol-2-yl)methyl)-1H-1,2,3- triazole-4-carboxamide |
| 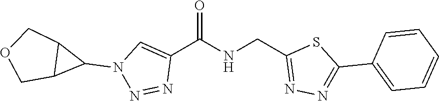 [SMILES: O=C(NCc1nnc(-c2ccccc2)s1)c1cn(C2C3COCC32)nn1] | 1-(3-oxabicyclo[3.1.0]hexan-6-yl)-N-((5-phenyl- 1,3,4-thiadiazol-2-yl)methyl)-1H-1,2,3-triazole-4- carboxamide |
| 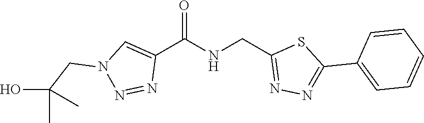 [SMILES: CC(C)(O)Cn1cc(C(=O)NCc2nnc(-c3ccccc3)s2)nn1] | 1-(2-hydroxy-2-methylpropyl)-N-((5-phenyl-1,3,4- thiadiazol-2-yl)methyl)-1H-1,2,3-triazole-4- carboxamide |
| 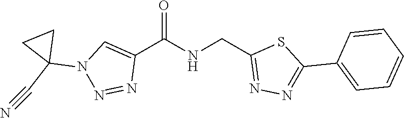 [SMILES: N#CC1(n2cc(C(=O)NCc3nnc(-c4ccccc4)s3)nn2)CC1] | 1-(1-cyanocyclopropyl)-N-((5-phenyl-1,3,4- thiadiazol-2-yl)methyl)-1H-1,2,3-triazole-4- carboxamide |
| 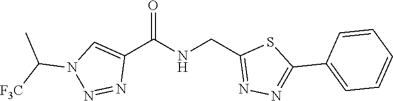 [SMILES: CC(n1cc(C(=O)NCc2nnc(-c3ccccc3)s2)nn1)C(F)(F)F] | N-((5-phenyl-1,3,4-thiadiazol-2-yl)methyl)-1- (1,1,1-trifluoropropan-2-yl)-1H-1,2,3-triazole-4- carboxamide |
| 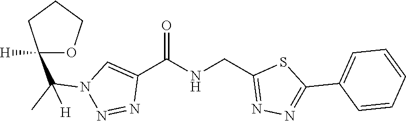 [SMILES: CC([C@H]1CCCO1)n1cc(C(=O)NCc2nnc(-c3ccccc3)s2)nn1] | N-((5-phenyl-1,3,4-thiadiazol-2-yl)methyl)-1-(1- ((R)-tetrahydrofuran-2-yl)ethyl)-1H-1,2,3-triazole- 4-carboxamide |
| 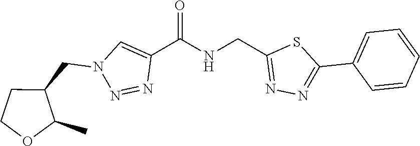 [SMILES: C[C@H]1OCC[C@H]1Cn1cc(C(=O)NCc2nnc(-c3ccccc3)s2)nn1] | 1-(((2R,3S)-2-methyltetrahydrofuran-3-yl)methyl)- N-((5-phenyl-1,3,4-thiadiazol-2-yl)methyl)-1H- 1,2,3-triazole-4-carboxamide |
| 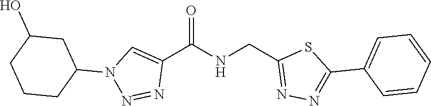 [SMILES: O=C(NCc1nnc(-c2ccccc2)s1)c1cn(C2CCCC(O)C2)nn1] | 1-(3-hydroxycyclohexyl)-N-((5-phenyl-1,3,4- thiadiazol-2-yl)methyl)-1H-1,2,3-triazole-4- carboxamide |
| 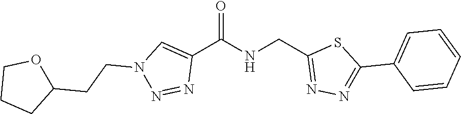 [SMILES: O=C(NCc1nnc(-c2ccccc2)s1)c1cn(CCC2CCCO2)nn1] | N-((5-phenyl-1,3,4-thiadiazol-2-yl)methyl)-1-(2- (tetrahydrofuran-2-yl)ethyl)-1H-1,2,3-triazole-4- carboxamide |
| 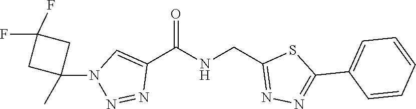 [SMILES: CC1(n2cc(C(=O)NCc3nnc(-c4ccccc4)s3)nn2)CC(F)(F)C1] | 1-(3,3-difluoro-1-methylcyclobutyl)-N-((5-phenyl- 1,3,4-thiadiazol-2-yl)methyl)-1H-1,2,3-triazole-4- carboxamide |
| 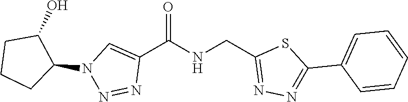 [SMILES: O=C(NCc1nnc(-c2ccccc2)s1)c1cn([C@H]2CCC[C@@H]2O)nn1] | 1-((1S,2S)-2-hydroxycyclopentyl)-N-((5-phenyl- 1,3,4-thiadiazol-2-yl)methyl)-1H-1,2,3-triazole-4- carboxamide |
| 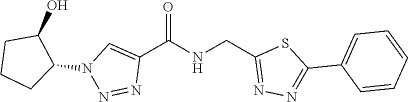 [SMILES: O=C(NCc1nnc(-c2ccccc2)s1)c1cn([C@@H]2CCC[C@H]2O)nn1] | 1-((1R,2R)-2-hydroxycyclopentyl)-N-((5-phenyl- 1,3,4-thiadiazol-2-yl)methyl)-1H-1,2,3-triazole-4- carboxamide |
| 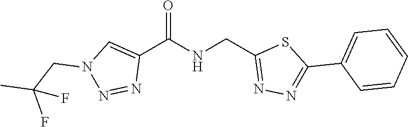 [SMILES: CC(F)(F)Cn1cc(C(=O)NCc2nnc(-c3ccccc3)s2)nn1] | 1-(2,2-difluoropropyl)-N-((5-phenyl-1,3,4- thiadiazol-2-yl)methyl)-1H-1,2,3-triazole-4- carboxamide |
| 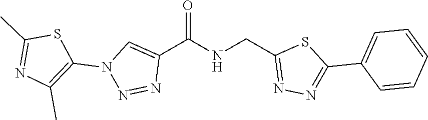 [SMILES: Cc1nc(C)c(-n2cc(C(=O)NCc3nnc(-c4ccccc4)s3)nn2)s1] | 1-(2,4-dimethylthiazol-5-yl)-N-((5-phenyl-1,3,4- thiadiazol-2-yl)methyl)-1H-1,2,3-triazole-4- carboxamide |
| 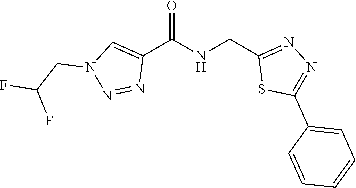 [SMILES: O=C(NCc1nnc(-c2ccccc2)s1)c1cn(CC(F)F)nn1] | Preparation of N-((5-phenyl-1,3,4-thiadiazol-2- yl)methyl)-1-(2,2,2-trifluoroethyl)-1H-1,2,3- triazole-4-carboxamide |
| 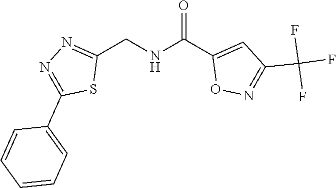 [SMILES: O=C(NCc1nnc(-c2ccccc2)s1)c1cc(C(F)(F)F)no1] | N-[(5-phenyl-1,3,4-thiadiazol-2-yl)methyl]-3- (trifluoromethyl)-1,2-oxazole-5-carboxamide |
| 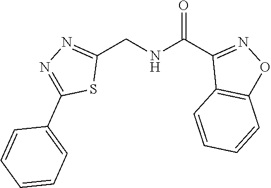 [SMILES: O=C(NCc1nnc(-c2ccccc2)s1)c1noc2ccccc12] | N-[(5-phenyl-1,3,4-thiadiazol-2-yl)methyl]-1,2- benzoxazole-3-carboxamide |
| 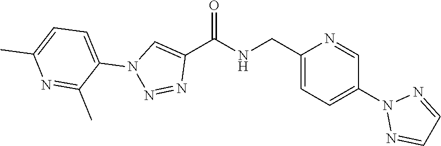 [SMILES: Cc1ccc(-n2cc(C(=O)NCc3ccc(-n4nccn4)cn3)nn2)c(C)n1] | 1-(2,6-dimethylpyridin-3-yl)-N-{[5-(2H-1,2,3- triazol-2-yl)pyridin-2-yl]methyl}-1H-1,2,3-triazole- 4-carboxamide |
| 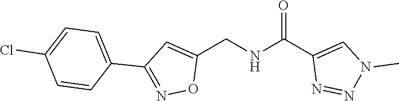 [SMILES: Cn1cc(C(=O)NCc2cc(-c3ccc(Cl)cc3)no2)nn1] | N-{[3-(4-chlorophenyl)-1,2-oxazol-5-yl]methyl}-1- methyl-1H-1,2,3-triazole-4-carboxamide |
| 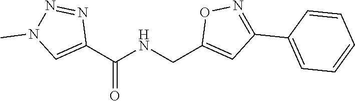 [SMILES: Cn1cc(C(=O)NCc2cc(-c3ccccc3)no2)nn1] | 1-methyl-N-[(3-phenyl-1,2-oxazol-5-yl)methyl]- 1H-1,2,3-triazole-4-carboxamide |
| 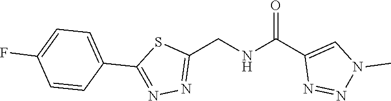 [SMILES: Cn1cc(C(=O)NCc2nnc(-c3ccc(F)cc3)s2)nn1] | N-{[5-(4-fluorophenyl)-1,3,4-thiadiazol-2- yl]methyl}-1-methyl-1H-1,2,3-triazole-4- carboxamide |
| 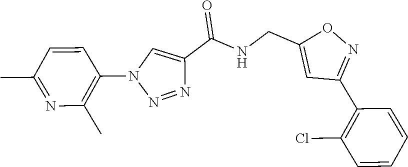 [SMILES: Cc1ccc(-n2cc(C(=O)NCc3cc(-c4ccccc4Cl)no3)nn2)c(C)n1] | N-{[3-(2-chlorophenyl)-1,2-oxazol-5-yl]methyl}-1- (2,6-dimethylpyridin-3-yl)-1H-1,2,3-triazole-4- carboxamide |
| 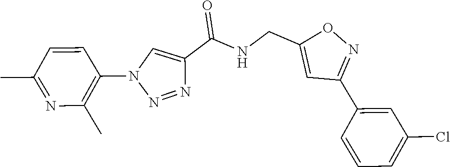 [SMILES: Cc1ccc(-n2cc(C(=O)NCc3cc(-c4cccc(Cl)c4)no3)nn2)c(C)n1] | N-{[3-(3-chlorophenyl)-1,2-oxazol-5-yl]methyl}-1- (2,6-dimethylpyridin-3-yl)-1H-1,2,3-triazole-4- carboxamide |
| 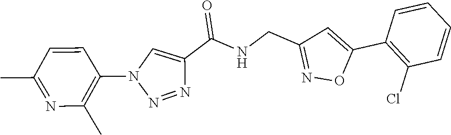 [SMILES: Cc1ccc(-n2cc(C(=O)NCc3cc(-c4ccccc4Cl)on3)nn2)c(C)n1] | N-{[5-(2-chlorophenyl)-1,2-oxazol-3-yl]methyl}-1- (2,6-dimethylpyridin-3-yl)-1H-1,2,3-triazole-4- carboxamide |
| 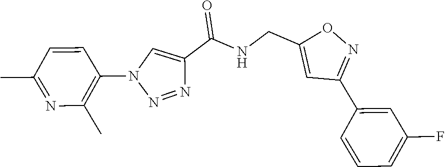 [SMILES: Cc1ccc(-n2cc(C(=O)NCc3cc(-c4cccc(F)c4)no3)nn2)c(C)n1] | 1-(2,6-dimethylpyridin-3-yl)-N-{[3-(3- fluorophenyl)-1,2-oxazol-5-yl]methyl}-1H-1,2,3- triazole-4-carboxamide |
| 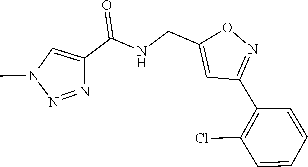 [SMILES: Cn1cc(C(=O)NCc2cc(-c3ccccc3Cl)no2)nn1] | N-{[3-(2-chlorophenyl)-1,2-oxazol-5-yl]methyl}-1- methyl-1H-1,2,3-triazole-4-carboxamide |
| 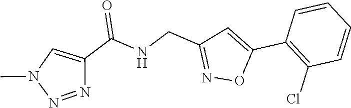 [SMILES: Cn1cc(C(=O)NCc2cc(-c3ccccc3Cl)on2)nn1] | N-{[5-(2-chlorophenyl)-1,2-oxazol-3-yl]methyl}-1- methyl-1H-1,2,3-triazole-4-carboxamide |
| 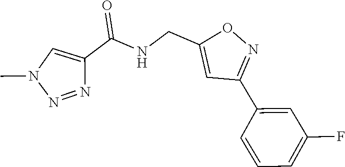 [SMILES: Cn1cc(C(=O)NCc2cc(-c3cccc(F)c3)no2)nn1] | N-{[3-(3-fluorophenyl)-1,2-oxazol-5-yl]methyl}-1- methyl-1H-1,2,3-triazole-4-carboxamide |
| 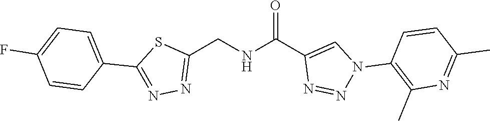 [SMILES: Cc1ccc(-n2cc(C(=O)NCc3nnc(-c4ccc(F)cc4)s3)nn2)c(C)n1] | 1-(2,6-dimethylpyridin-3-yl)-N-{[5-(4- fluorophenyl)-1,3,4-thiadiazol-2-yl]methyl}-1H- 1,2,3-triazole-4-carboxamide |
| 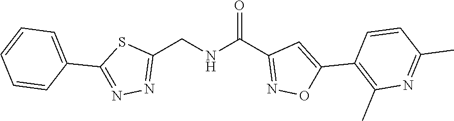 [SMILES: Cc1ccc(-c2cc(C(=O)NCc3nnc(-c4ccccc4)s3)no2)c(C)n1] | 5-(2,6-dimethylpyridin-3-yl)-N-[(5-phenyl-1,3,4- thiadiazol-2-yl)methyl]-1,2-oxazole-3-carboxamide |
| 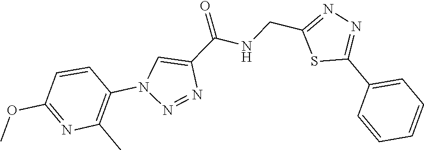 [SMILES: COc1ccc(-n2cc(C(=O)NCc3nnc(-c4ccccc4)s3)nn2)c(C)n1] | 1-(6-methoxy-2-methylpyridin-3-yl)- N-((5-phenyl-1,3,4-thiadiazol-2-yl)methyl)-1H- 1,2,3-triazole-4-carboxamide |
| 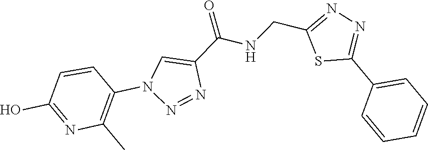 [SMILES: Cc1nc(O)ccc1-n1cc(C(=O)NCc2nnc(-c3ccccc3)s2)nn1] | 1-(6-hydroxy-2-methylpyridin-3-yl)-N- ((5-phenyl-1,3,4-thiadiazol-2-yl)methyl)- 1H-1,2,3-triazole-4-carboxamide |
| 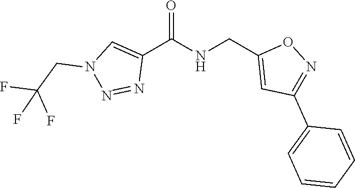 [SMILES: O=C(NCc1cc(-c2ccccc2)no1)c1cn(CC(F)(F)F)nn1] | N-((3-phenylisoxazol-5-yl)methyl)-1-(2,2,2- trifluoroethyl)-1H-1,2,3-triazole-4-carboxamide |
| 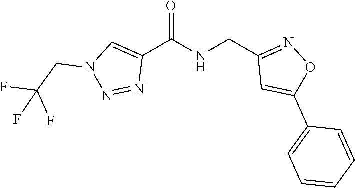 [SMILES: O=C(NCc1cc(-c2ccccc2)on1)c1cn(CC(F)(F)F)nn1] | N-((5-phenylisoxazol-3-yl)methyl)-1-(2,2,2- trifluoroethyl)-1H-1,2,3-triazole-4-carboxamide |
| 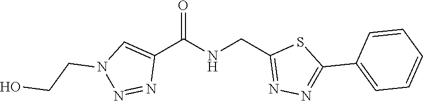 [SMILES: O=C(NCc1nnc(-c2ccccc2)s1)c1cn(CCO)nn1] | 1-(2-hydroxyethyl)-N-((5-phenyl-1,3,4-thiadiazol- 2-yl)methyl)-1H-1,2,3-triazole-4-carboxamide |
| 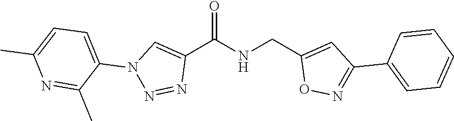 [SMILES: Cc1ccc(-n2cc(C(=O)NCc3cc(-c4ccccc4)no3)nn2)c(C)n1] | 1-(2,6-dimethylpyridin-3-yl)-N-((3-phenylisoxazol- 5-yl)methyl)-1H-1,2,3-triazole-4-carboxamide |
| 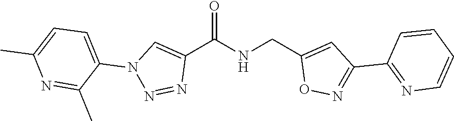 [SMILES: Cc1ccc(-n2cc(C(=O)NCc3cc(-c4ccccn4)no3)nn2)c(C)n1] | 1-(2,6-dimethylpyridin-3-yl)-N-((3-(pyridin-2- yl)isoxazol-5-yl)methyl)-1H-1,2,3-triazole-4- carboxamide |
| 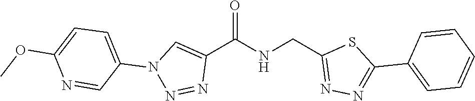 [SMILES: COc1ccc(-n2cc(C(=O)NCc3nnc(-c4ccccc4)s3)nn2)cn1] | 1-(6-methoxypyridin-3-yl)-N-((5-phenyl-1,3,4- thiadiazol-2-yl)methyl)-1H-1,2,3-triazole-4- carboxamide |
| 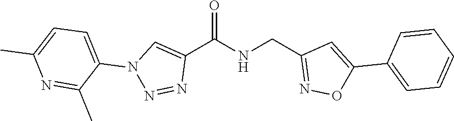 [SMILES: Cc1ccc(-n2cc(C(=O)NCc3cc(-c4ccccc4)on3)nn2)c(C)n1] | 1-(2,6-dimethylpyridin-3-yl)-N-((5-phenylisoxazol- 3-yl)methyl)-1H-1,2,3-triazole-4-carboxamide |
| 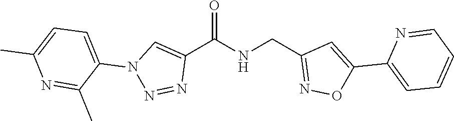 [SMILES: Cc1ccc(-n2cc(C(=O)NCc3cc(-c4ccccn4)on3)nn2)c(C)n1] | 1-(2,6-dimethylpyridin-3-yl)-N-((5-(pyridin-2- yl)isoxazol-3-yl)methyl)-1H-1,2,3-triazole-4- carboxamide |
| 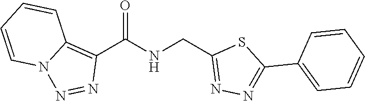 [SMILES: O=C(NCc1nnc(-c2ccccc2)s1)c1nnn2ccccc12] | N-((5-phenyl-1,3,4-thiadiazol-2-yl)methyl)- [1,2,3]triazolo[1,5-a]pyridine-3-carboxamide |
| 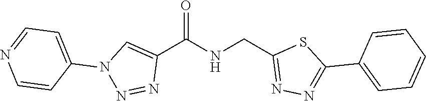 [SMILES: O=C(NCc1nnc(-c2ccccc2)s1)c1cn(-c2ccncc2)nn1] | N-((5-phenyl-1,3,4-thiadiazol-2-yl)methyl)-1- (pyridin-4-yl)-1H-1,2,3-triazole-4-carboxamide |
| 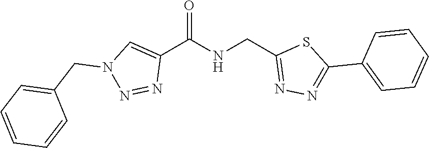 [SMILES: O=C(NCc1nnc(-c2ccccc2)s1)c1cn(Cc2ccccc2)nn1] | 1-benzyl-N-((5-phenyl-1,3,4-thiadiazol-2- yl)methyl)-1H-1,2,3-triazole-4-carboxamide |
| 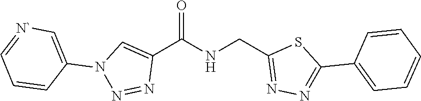 | N-((5-phenyl-1,3,4-thiadiazol-2-yl)methyl)-1- (pyridin-3-yl)-1H-1,2,3-triazole-4-carboxamide |
| 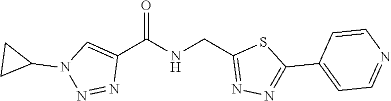 [SMILES: O=C(NCc1nnc(-c2ccncc2)s1)c1cn(C2CC2)nn1] | 1-cyclopropyl-N-((5-(pyridin-4-yl)-1,3,4- thiadiazol-2-yl)methyl)-1H-1,2,3-triazole-4- carboxamide |
| 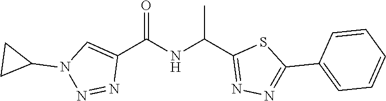 [SMILES: CC(NC(=O)c1cn(C2CC2)nn1)c1nnc(-c2ccccc2)s1] | 1-cyclopropyl-N-(1-(5-phenyl-1,3,4-thiadiazol-2- yl)ethyl)-1H-1,2,3-triazole-4-carboxamide |
| 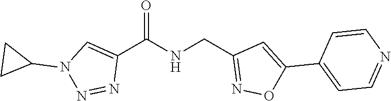 [SMILES: O=C(NCc1cc(-c2ccncc2)on1)c1cn(C2CC2)nn1] | 1-cyclopropyl-N-((5-(pyridin-4-yl)isoxazol-3- yl)methyl)-1H-1,2,3-triazole-4-carboxamide |
| 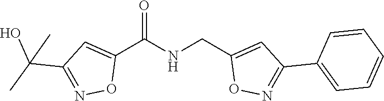 [SMILES: CC(C)(O)c1cc(C(=O)NCc2cc(-c3ccccc3)no2)on1] | 3-(2-hydroxypropan-2-yl)-N-((3-phenylisoxazol-5- yl)methyl)isoxazole-5-carboxamide |
| 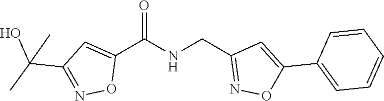 [SMILES: CC(C)(O)c1cc(C(=O)NCc2cc(-c3ccccc3)on2)on1] | 3-(2-hydroxypropan-2-yl)-N-((5-phenylisoxazol-3- yl)methyl)isoxazole-5-carboxamide |
| 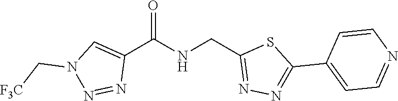 [SMILES: O=C(NCc1nnc(-c2ccncc2)s1)c1cn(CC(F)(F)F)nn1] | N-((5-(pyridin-4-yl)-1,3,4-thiadiazol-2-yl)methyl)- 1-(2,2,2-trifluoroethyl)-1H-1,2,3-triazole-4- carboxamide |
| 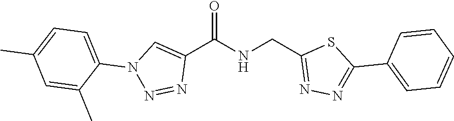 [SMILES: Cc1ccc(-n2cc(C(=O)NCc3nnc(-c4ccccc4)s3)nn2)c(C)c1] | 1-(2,4-dimethylphenyl)-N-((5-(pyridin-4-yl)-1,3,4- thiadiazol-2-yl)methyl)-1H-1,2,3-triazole-4- carboxamide |
| 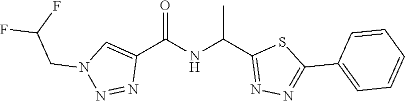 [SMILES: CC(NC(=O)c1cn(CC(F)F)nn1)c1nnc(-c2ccccc2)s1] | 1-(2,2-difluoroethyl)-N-(1-(5-phenyl-1,3,4- thiadiazol-2-yl)ethyl)-1H-1,2,3-triazole-4- carboxamide |
| 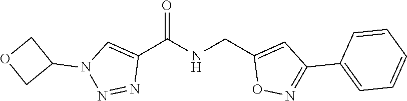 [SMILES: O=C(NCc1cc(-c2ccccc2)no1)c1cn(C2COC2)nn1] | 1-(oxetan-3-yl)-N-((3-phenylisoxazol-5-yl)methyl)- 1H-1,2,3-triazole-4-carboxamide |
| 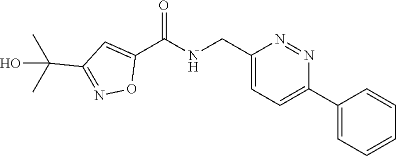 [SMILES: CC(C)(O)c1cc(C(=O)NCc2ccc(-c3ccccc3)nn2)on1] | 3-(2-hydroxypropan-2-yl)-N-((6-phenylpyridazin- 3-yl)methyl)isoxazole-5-carboxamide |
| 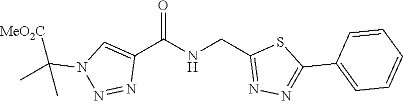 [SMILES: COC(=O)C(C)(C)n1cc(C(=O)NCc2nnc(-c3ccccc3)s2)nn1] | methyl 2-methyl-2-(4-(((5-phenyl-1,3,4-thiadiazol- 2-yl)methyl)carbamoyl)-1H-1,2,3-triazol-1- yl)propanoate |
| 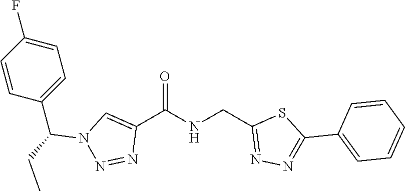 [SMILES: CC[C@H](c1ccc(F)cc1)n1cc(C(=O)NCc2nnc(-c3ccccc3)s2)nn1] | (R)-1-(1-(4-fluorophenyl)propyl)-N-((5-phenyl- 1,3,4-thiadiazol-2-yl)methyl)-1H-1,2,3-triazole-4- carboxamide |
| 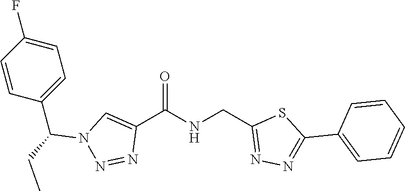 [SMILES: CC[C@H](c1ccc(F)cc1)n1cc(C(=O)NCc2nnc(-c3ccccc3)s2)nn1] | (S)-1-(1-(4-fluorophenyl)propyl)-N-((5-phenyl- 1,3,4-thiadiazol-2-yl)methyl)-1H-1,2,3-triazole-4- carboxamide |
| 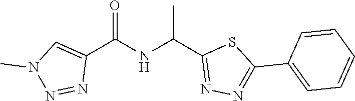 [SMILES: CC(NC(=O)c1cn(C)nn1)c1nnc(-c2ccccc2)s1] | 1-methyl-N-(1-(5-phenyl-1,3,4-thiadiazol-2- yl)ethyl)-1H-1,2,3-triazole-4-carboxamide |
| 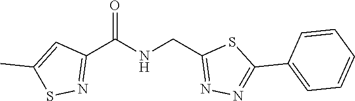 [SMILES: Cc1cc(C(=O)NCc2nnc(-c3ccccc3)s2)ns1] | 5-methyl-N-((5-phenyl-1,3,4-thiadiazol-2- yl)methyl)isothiazole-3-carboxamide |
| 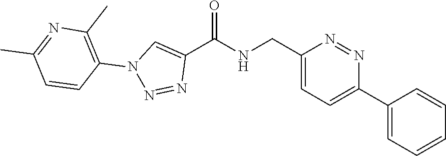 [SMILES: Cc1ccc(-n2cc(C(=O)NCc3ccc(-c4ccccc4)nn3)nn2)c(C)n1] | 1-(2,6-dimethylpyridin-3-yl)-N-((6- phenylpyridazin-3-yl)methyl)-1H-1,2,3-triazole-4- carboxamide |
| 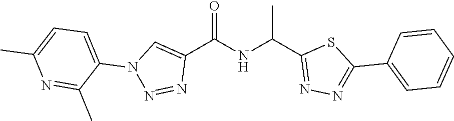 [SMILES: Cc1ccc(-n2cc(C(=O)NC(C)c3nnc(-c4ccccc4)s3)nn2)c(C)n1] | 1-(2,6-dimethylpyridin-3-yl)-N-(1-(5-phenyl-1,3,4- thiadiazol-2-yl)ethyl)-1H-1,2,3-triazole-4- carboxamide |
| 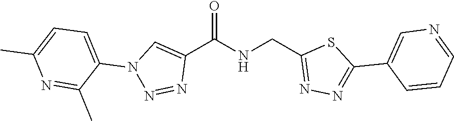 [SMILES: Cc1ccc(-n2cc(C(=O)NCc3nnc(-c4cccnc4)s3)nn2)c(C)n1] | 1-(2,6-dimethylpyridin-3-yl)-N-((5-(pyridin-3-yl)- 1,3,4-thiadiazol-2-yl)methyl)-1H-1,2,3-triazole-4- carboxamide |
| 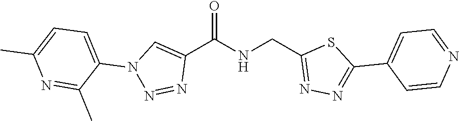 [SMILES: Cc1ccc(-n2cc(C(=O)NCc3nnc(-c4ccncc4)s3)nn2)c(C)n1] | 1-(2,6-dimethylpyridin-3-yl)-N-((5-(pyridin-4-yl)- 1,3,4-thiadiazol-2-yl)methyl)-1H-1,2,3-triazole-4- carboxamide |
| 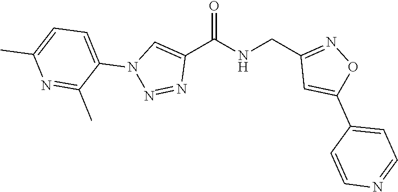 [SMILES: Cc1ccc(-n2cc(C(=O)NCc3cc(-c4ccncc4)on3)nn2)c(C)n1] | 1-(2,6-dimethylpyridin-3-yl)-N-((5-(pyridin-4- yl)isoxazol-3-yl)methyl)-1H-1,2,3-triazole-4- carboxamide |
| 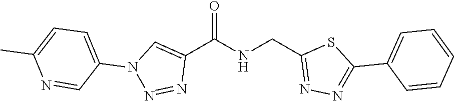 [SMILES: Cc1ccc(-n2cc(C(=O)NCc3nnc(-c4ccccc4)s3)nn2)cn1] | 1-(6-methylpyridin-3-yl)-N-((5-phenyl-1,3,4- thiadiazol-2-yl)methyl)-1H-1,2,3-triazole-4- carboxamide |
| 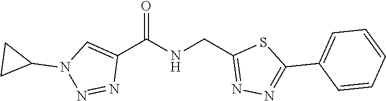 [SMILES: O=C(NCc1nnc(-c2ccccc2)s1)c1cn(C2CC2)nn1] | 1-cyclopropyl-N-((5-phenyl-1,3,4-thiadiazol-2- yl)methyl)-1H-1,2,3-triazole-4-carboxamide |
| 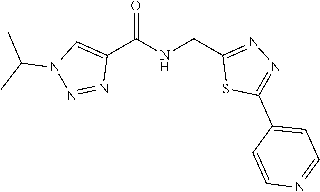 [SMILES: CC(C)n1cc(C(=O)NCc2nnc(-c3ccncc3)s2)nn1] | 1-isopropyl-N-((5-(pyridin-4-yl)-1,3,4-thiadiazol-2- yl)methyl)-1H-1,2,3-triazole-4-carboxamide |
| 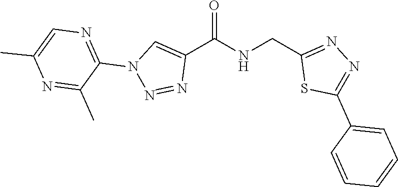 [SMILES: Cc1cnc(-n2cc(C(=O)NCc3nnc(-c4ccccc4)s3)nn2)c(C)n1] | 1-(3,5-dimethylpyrazin-2-yl)-N-((5-phenyl-1,3,4- thiadiazol-2-yl)methyl)-1H-1,2,3-triazole-4- carboxamide |
| 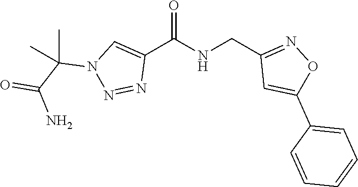 [SMILES: CC(C)(C(N)=O)n1cc(C(=O)NCc2cc(-c3ccccc3)on2)nn1] | 1-(1-amino-2-methyl-1-oxopropan-2-yl)-N-((5- phenylisoxazol-3-yl)methyl)-1H-1,2,3-triazole-4- carboxamide |
| 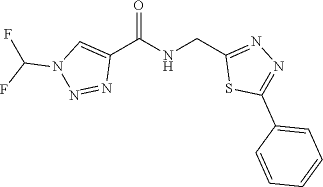 [SMILES: O=C(NCc1nnc(-c2ccccc2)s1)c1cn(C(F)F)nn1] | 1-(difluoromethyl)-N-((5-phenyl-1,3,4-thiadiazol- 2-yl)methyl)-1H-1,2,3-triazole-4-carboxamide |
| 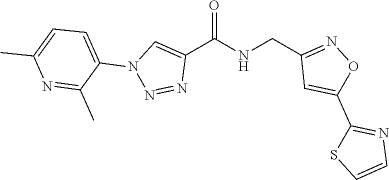 [SMILES: Cc1ccc(-n2cc(C(=O)NCc3cc(-c4nccs4)on3)nn2)c(C)n1] | 1-(2,6-dimethylpyridin-3-yl)-N-((5-(thiazol-2- yl)isoxazol-3-yl)methyl)-1H-1,2,3-triazole-4- carboxamide |
| 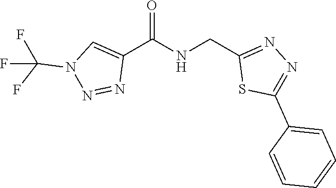 [SMILES: O=C(NCc1nnc(-c2ccccc2)s1)c1cn(C(F)(F)F)nn1] | N-((5-phenyl-1,3,4-thiadiazol-2-yl)methyl)-1- (trifluoromethyl)-1H-1,2,3-triazole-4-carboxamide |
| 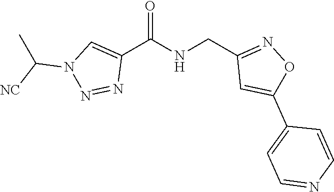 [SMILES: CC(C#N)n1cc(C(=O)NCc2cc(-c3ccncc3)on2)nn1] | 1-(1-cyanoethyl)-N-((5-(pyridin-4-yl)isoxazol-3- yl)methyl)-1H-1,2,3-triazole-4-carboxamide |
| 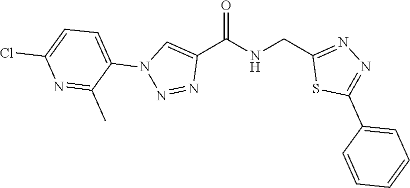 [SMILES: Cc1nc(Cl)ccc1-n1cc(C(=O)NCc2nnc(-c3ccccc3)s2)nn1] | 1-(6-chloro-2-methylpyridin-3-yl)-N-((5-phenyl- 1,3,4-thiadiazol-2-yl)methyl)-1H-1,2,3-triazole-4- carboxamide |
| 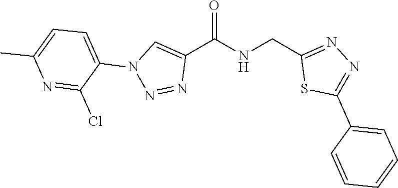 [SMILES: Cc1ccc(-n2cc(C(=O)NCc3nnc(-c4ccccc4)s3)nn2)c(Cl)n1] | 1-(2-chloro-6-methylpyridin-3-yl)-N-((5-phenyl- 1,3,4-thiadiazol-2-yl)methyl)-1H-1,2,3-triazole-4- carboxamide |
| 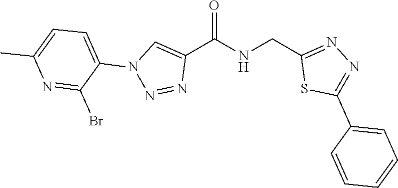 [SMILES: Cc1ccc(-n2cc(C(=O)NCc3nnc(-c4ccccc4)s3)nn2)c(Br)n1] | 1-(2-bromo-6-methylpyridin-3-yl)-N-((5-phenyl- 1,3,4-thiadiazol-2-yl)methyl)-1H-1,2,3-triazole-4- carboxamide |
| 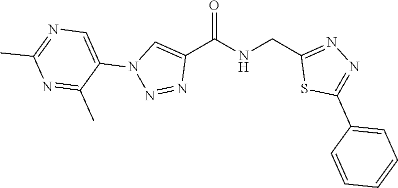 [SMILES: Cc1ncc(-n2cc(C(=O)NCc3nnc(-c4ccccc4)s3)nn2)c(C)n1] | 1-(2,4-dimethylpyrimidin-5-yl)-N-((5-phenyl-1,3,4- thiadiazol-2-yl)methyl)-1H-1,2,3-triazole-4- carboxamide |
| 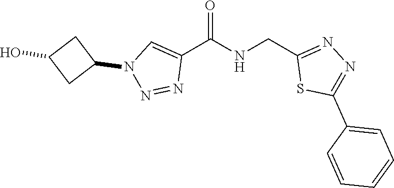 | 1-trans-3-hydroxycyclobutyl)-N-((5-phenyl-1,3,4- thiadiazol-2-yl)methyl)-1H-1,2,3-triazole-4- carboxamide |
| 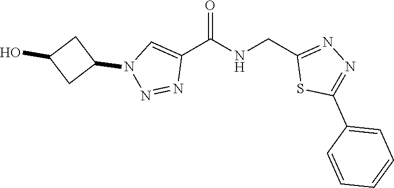 | 1-cis-3-hydroxycyclobutyl)-N-((5-phenyl-1,3,4- thiadiazol-2-yl)methyl)-1H-1,2,3-triazole-4- carboxamide |
| 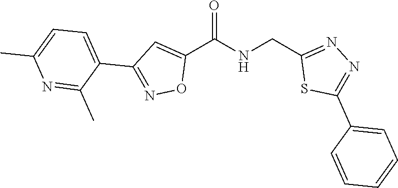 [SMILES: Cc1ccc(-c2cc(C(=O)NCc3nnc(-c4ccccc4)s3)on2)c(C)n1] | 3-(2,6-dimethylpyridin-3-yl)-N-((5-phenyl-1,3,4- thiadiazol-2-yl)methyl)isoxazole-5-carboxamide |
| 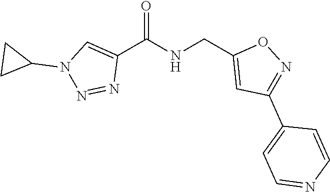 [SMILES: O=C(NCc1cc(-c2ccncc2)no1)c1cn(C2CC2)nn1] | 1-cyclopropyl-N-((3-(pyridin-4-yl)isoxazol-5- yl)methyl)-1H-1,2,3-triazole-4-carboxamide |
| 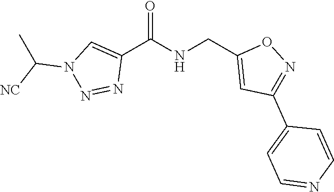 [SMILES: CC(C#N)n1cc(C(=O)NCc2cc(-c3ccncc3)no2)nn1] | 1-(1-cyanoethyl)-N-((3-(pyridin-4-yl)isoxazol-5- yl)methyl)-1H-1,2,3-triazole-4-carboxamide |
| 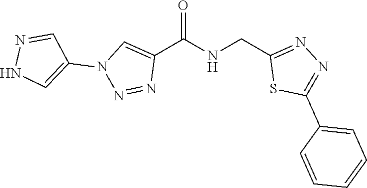 [SMILES: O=C(NCc1nnc(-c2ccccc2)s1)c1cn(-c2cn[nH]c2)nn1] | N-((5-phenyl-1,3,4-thiadiazol-2-yl)methyl)-1-(1H- pyrazol-4-yl)-1H-1,2,3-triazole-4-carboxamide |
| 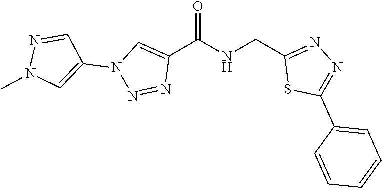 [SMILES: Cn1cc(-n2cc(C(=O)NCc3nnc(-c4ccccc4)s3)nn2)cn1] | 1-(1-methyl-1H-pyrazol-4-yl)-N-((5-phenyl-1,3,4- thiadiazol-2-yl)methyl)-1H-1,2,3-triazole-4- carboxamide |
| 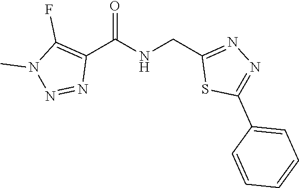 [SMILES: Cn1nnc(C(=O)NCc2nnc(-c3ccccc3)s2)c1F] | 5-fluoro-1-methyl-N-((5-phenyl-1,3,4-thiadiazol-2- yl)methyl)-1H-1,2,3-triazole-4-carboxamide |
| 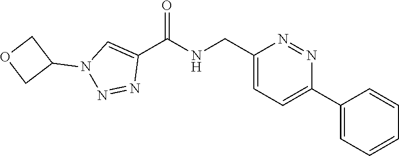 [SMILES: O=C(NCc1ccc(-c2ccccc2)nn1)c1cn(C2COC2)nn1] | 1-(oxetan-3-yl)-N-((3-phenylisoxazol-5-yl)methyl)- 1H-1,2,3-triazole-4-carboxamide |
| 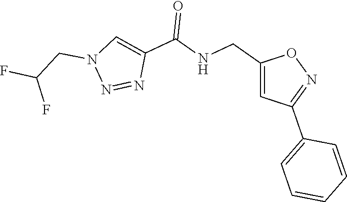 [SMILES: O=C(NCc1cc(-c2ccccc2)no1)c1cn(CC(F)F)nn1] | 1-(2,2-difluoroethyl)-N-((3-phenylisoxazol-5- yl)methyl)-1H-1,2,3-triazole-4-carboxamide |
| 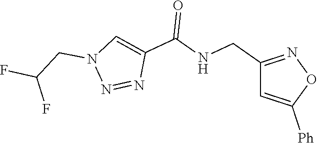 [SMILES: O=C(NCc1cc(-c2ccccc2)on1)c1cn(CC(F)F)nn1] | 1-(2,2-difluoroethyl)-N-((5-phenylisoxazol-3- yl)methyl)-1H-1,2,3-triazole-4-carboxamide |
| 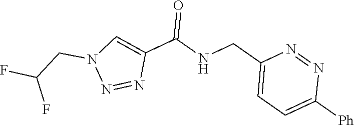 [SMILES: O=C(NCc1ccc(-c2ccccc2)nn1)c1cn(CC(F)F)nn1] | 1-(2,2-difluoroethyl)-N-((6-phenylpyridazin-3- yl)methyl)-1H-1,2,3-triazole-4-carboxamide |
| 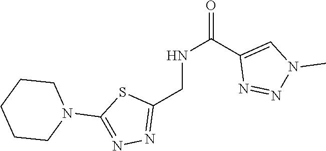 [SMILES: Cn1cc(C(=O)NCc2nnc(N3CCCCC3)s2)nn1] | 1-methyl-N-((5-(piperidin-1-yl)-1,3,4-thiadiazol-2- yl)methyl)-1H-1,2,3-triazole-4-carboxamide |
| 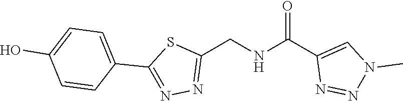 [SMILES: Cn1cc(C(=O)NCc2nnc(-c3ccc(O)cc3)s2)nn1] | N-[[5-(4-hydroxyphenyl)-1,3,4-thiadiazol-2- yl]methyl]-1-methyl-triazole-4-carboxamide |
| 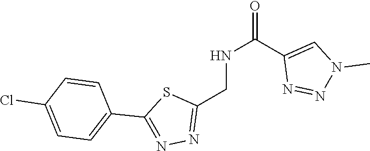 [SMILES: Cn1cc(C(=O)NCc2nnc(-c3ccc(Cl)cc3)s2)nn1] | N-((5-(4-chlorophenyl)-1,3,4-thiadiazol-2- yl)methyl)-1-methyl- 1H-1,2,3-triazole-4-carboxamide |
| 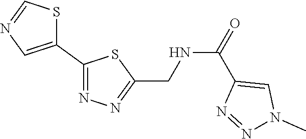 [SMILES: Cn1cc(C(=O)NCc2nnc(-c3cncs3)s2)nn1] | 1-methyl-N-((5-(thiazol-5-yl)-1,3,4-thiadiazol-2- yl)methyl)-1H-1,2,3-triazole-4-carboxamide |
| 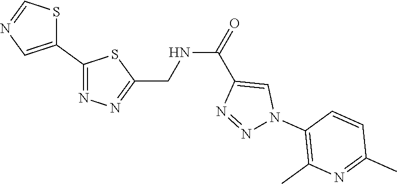 [SMILES: Cc1ccc(-n2cc(C(=O)NCc3nnc(-c4cncs4)s3)nn2)c(C)n1] | 1-(2,6-dimethylpyridin-3-yl)-N-((5-(thiazol- 5-yl)-1,3,4-thiadiazol-2-yl)methyl)-1H-1,2,3- triazole-4-carboxamide |
| 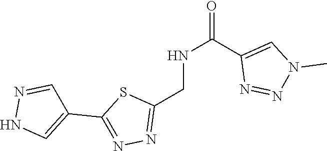 [SMILES: Cn1cc(C(=O)NCc2nnc(-c3cn[nH]c3)s2)nn1] | N-((5-(1H-pyrazol-4-yl)-1,3,4-thiadiazol-2- yl)methyl)-1-methyl-1H-1,2,3-triazole-4- carboxamide |
| 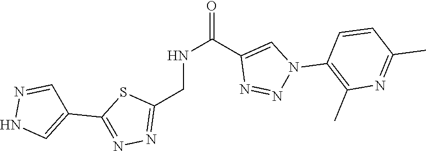 [SMILES: Cc1ccc(-n2cc(C(=O)NCc3nnc(-c4cn[nH]c4)s3)nn2)c(C)n1] | N-((5-(1H-pyrazol-4-yl)-1,3,4-thiadiazol-2- yl)methyl)-1-(2,6-dimethylpyridin-3-yl)-1H-1,2,3- triazole-4-carboxamide |
| 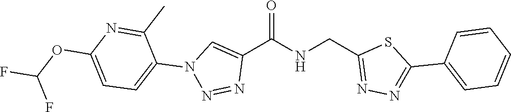 [SMILES: Cc1nc(OC(F)F)ccc1-n1cc(C(=O)NCc2nnc(-c3ccccc3)s2)nn1] | 1-(6-(difluoromethoxy)-2-methylpyridin-3-yl)-N- ((5-phenyl-1,3,4-thiadiazol-2-yl)methyl)-1H-1,2,3- triazole-4-carboxamide |
| 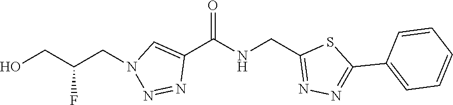 [SMILES: O=C(NCc1nnc(-c2ccccc2)s1)c1cn(C[C@H](F)CO)nn1] | (S)-1-(2-fluoro-3-hydroxypropyl)-N-((5-phenyl- 1,3,4-thiadiazol-2-yl)methyl)-1H-1,2,3-triazole-4- carboxamide |
| 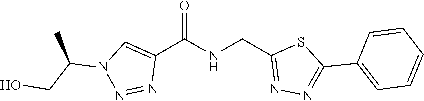 [SMILES: C[C@H](CO)n1cc(C(=O)NCc2nnc(-c3ccccc3)s2)nn1] | (R)-1-(1-hydroxypropan-2-yl)-N-((5-phenyl-1,3,4- thiadiazol-2-yl)methyl)-1H-1,2,3-triazole-4- carboxamide |
| 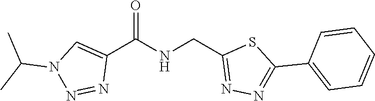 [SMILES: CC(C)n1cc(C(=O)NCc2nnc(-c3ccccc3)s2)nn1] | 1-isopropyl-N-((5-phenyl-1,3,4-thiadiazol-2- yl)methyl)-1H-1,2,3-triazole-4-carboxamide |
| 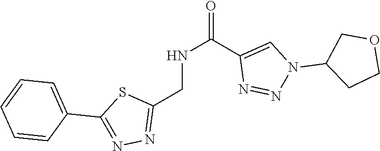 [SMILES: O=C(NCc1nnc(-c2ccccc2)s1)c1cn(C2CCOC2)nn1] | N-((5-phenyl-1,3,4-thiadiazol-2-yl)methyl)-1- (tetrahydrofuran-3-yl)-1H-1,2,3-triazole-4- carboxamide |
| 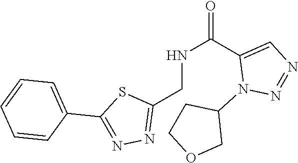 [SMILES: O=C(NCc1nnc(-c2ccccc2)s1)c1cnnn1C1CCOC1] | N-((5-phenyl-1,3,4-thiadiazol-2-yl)methyl)-1- (tetrahydrofuran-3-yl)-1H-1,2,3-triazole-5- carboxamide |
| 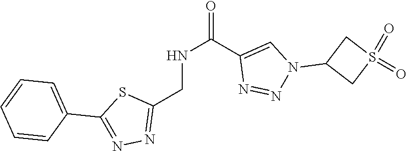 [SMILES: O=C(NCc1nnc(-c2ccccc2)s1)c1cn(C2CS(=O)(=O)C2)nn1] | 1-(1,1-dioxidothietan-3-yl)-N-((5-phenyl-1,3,4- thiadiazol-2-yl)methyl)-1H-1,2,3-triazole-4- carboxamide |
| 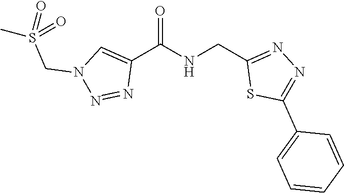 [SMILES: CS(=O)(=O)Cn1cc(C(=O)NCc2nnc(-c3ccccc3)s2)nn1] | (1-((methylsulfonyl)methyl)-N-((5-phenyl-1,3,4- thiadiazol-2-yl)methyl)-1H-1,2,3-triazole-4- carboxamide |
| 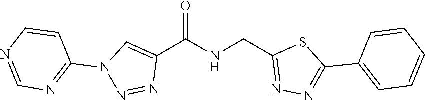 [SMILES: O=C(NCc1nnc(-c2ccccc2)s1)c1cn(-c2ccncn2)nn1] | N-((5-phenyl-1,3,4-thiadiazol-2-yl)methyl)-1- (pyrimidin-4-yl)-1H-1,2,3-triazole-4-carboxamide |
| 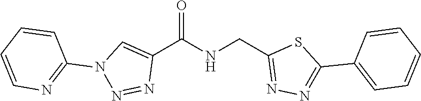 [SMILES: O=C(NCc1nnc(-c2ccccc2)s1)c1cn(-c2ccccn2)nn1] | N-((5-phenyl-1,3,4-thiadiazol-2-yl)methyl)-1- (pyridin-2-yl)-1H-1,2,3-triazole-4-carboxamide |
| 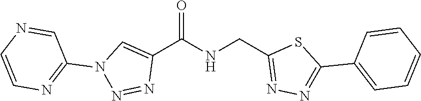 [SMILES: O=C(NCc1nnc(-c2ccccc2)s1)c1cn(-c2cnccn2)nn1] | N-((5-phenyl-1,3,4-thiadiazol-2-yl)methyl)-1- (pyrazin-2-yl)-1H-1,2,3-triazole-4-carboxamide |
| 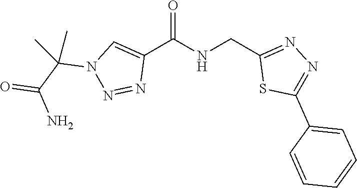 [SMILES: CC(C)(C(N)=O)n1cc(C(=O)NCc2nnc(-c3ccccc3)s2)nn1] | 1-(1-amino-2-methyl-1-oxopropan-2-yl)-N-((5- phenyl-1,3,4-thiadiazol-2-yl)methyl)-1H-1,2,3- triazole-4-carboxamide |
| 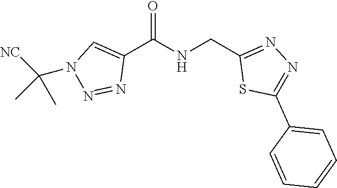 [SMILES: CC(C)(C#N)n1cc(C(=O)NCc2nnc(-c3ccccc3)s2)nn1] | 1-(2-cyanopropan-2-yl)-N-((5-phenyl-1,3,4- thiadiazol-2-yl)methyl)-1H-1,2,3-triazole-4- carboxamide |
| 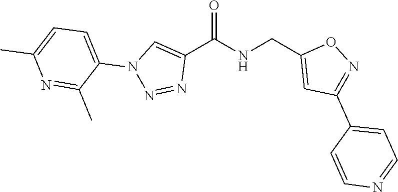 [SMILES: Cc1ccc(-n2cc(C(=O)NCc3cc(-c4ccncc4)no3)nn2)c(C)n1] | 1-(2,6-dimethylpyridin-3-yl)-N-((3-(pyridin-4- yl)isoxazol-5-yl)methyl)-1H-1,2,3-triazole-4- carboxamide |
| 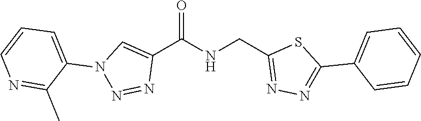 [SMILES: Cc1ncccc1-n1cc(C(=O)NCc2nnc(-c3ccccc3)s2)nn1] | 1-(2-methylpyridin-3-yl)-N-((5-phenyl-1,3,4- thiadiazol-2-yl)methyl)-1H-1,2,3-triazole-4- carboxamide |
| 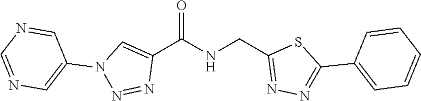 [SMILES: O=C(NCc1nnc(-c2ccccc2)s1)c1cn(-c2cncnc2)nn1] | N-((5-phenyl-1,3,4-thiadiazol-2-yl)methyl)-1- (pyrimidin-5-yl)-1H-1,2,3-triazole-4-carboxamide |
| 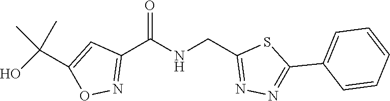 [SMILES: CC(C)(O)c1cc(C(=O)NCc2nnc(-c3ccccc3)s2)no1] | 5-(2-hydroxypropan-2-yl)-N-((5-phenyl-1,3,4- thiadiazol-2-yl)methyl)isoxazole-3-carboxamide |
| 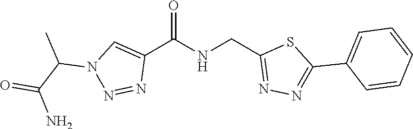 [SMILES: CC(C(N)=O)n1cc(C(=O)NCc2nnc(-c3ccccc3)s2)nn1] | 1-(1-amino-1-oxopropan-2-yl)-N-((5-phenyl-1,3,4- thiadiazol-2-yl)methyl)-1H-1,2,3-triazole-4- carboxamide |
| 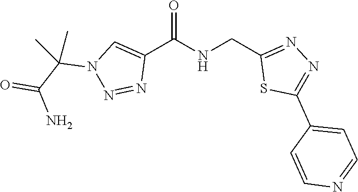 [SMILES: CC(C)(C(N)=O)n1cc(C(=O)NCc2nnc(-c3ccncc3)s2)nn1] | 1-(1-amino-2-methyl-1-oxopropan-2-yl)-N-((5- (pyridin-4-yl)-1,3,4-thiadiazol-2-yl)methyl)-1H- 1,2,3-triazole-4-carboxamide |
| 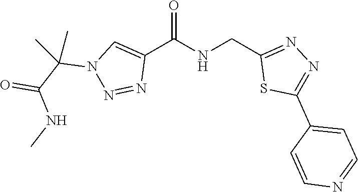 [SMILES: CNC(=O)C(C)(C)n1cc(C(=O)NCc2nnc(-c3ccncc3)s2)nn1] | 1-(2-methyl-1-(methylamino)-1-oxopropan-2-yl)- N-((5-(pyridin-4-yl)-1,3,4-thiadiazol-2-yl)methyl)- 1H-1,2,3-triazole-4-carboxamide |
| 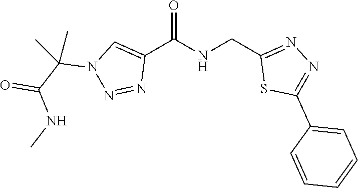 [SMILES: CNC(=O)C(C)(C)n1cc(C(=O)NCc2nnc(-c3ccccc3)s2)nn1] | 1-(2-methyl-1-(methylamino)-1-oxopropan-2-yl)- N-((5-phenyl-1,3,4-thiadiazol-2-yl)methyl)-1H- 1,2,3-triazole-4-carboxamide |
| 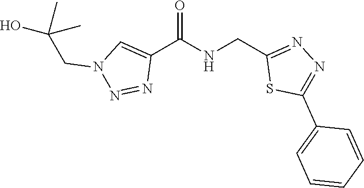 [SMILES: CC(C)(O)Cn1cc(C(=O)NCc2nnc(-c3ccccc3)s2)nn1] | 1-(2-hydroxy-2-methylpropyl)-N-((5-phenyl-1,3,4- thiadiazol-2-yl)methyl)-1H-1,2,3-triazole-4- carboxamide |
| 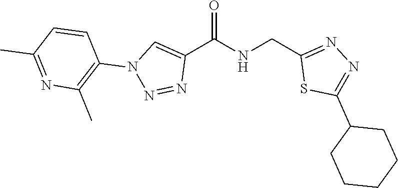 [SMILES: Cc1ccc(-n2cc(C(=O)NCc3nnc(C4CCCCC4)s3)nn2)c(C)n1] | N-((5-cyclohexyl-1,3,4-thiadiazol-2-yl)methyl)-1- (2,6-dimethylpyridin-3-yl)-1H-1,2,3-triazole-4- carboxamide |
| 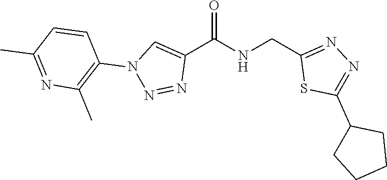 [SMILES: Cc1ccc(-n2cc(C(=O)NCc3nnc(C4CCCC4)s3)nn2)c(C)n1] | N-((5-cyclopentyl-1,3,4-thiadiazol-2-yl)methyl)-1- (2,6-dimethylpyridin-3-yl)-1H-1,2,3-triazole-4- carboxamide |
| 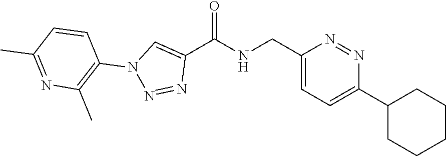 [SMILES: Cc1ccc(-n2cc(C(=O)NCc3ccc(C4CCCCC4)nn3)nn2)c(C)n1] | N-((6-cyclohexylpyridazin-3-yl)methyl)-1-(2,6- dimethylpyridin-3-yl)-1H-1,2,3-triazole-4- carboxamide |
| 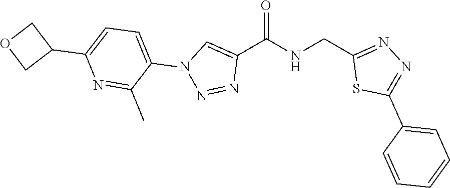 [SMILES: Cc1nc(C2COC2)ccc1-n1cc(C(=O)NCc2nnc(-c3ccccc3)s2)nn1] | 1-(2-methyl-6-(oxetan-3-yl)pyridin-3-yl)-N-((5- phenyl-1,3,4-thiadiazol-2-yl)methyl)-1H-1,2,3- triazole-4-carboxamide |
| 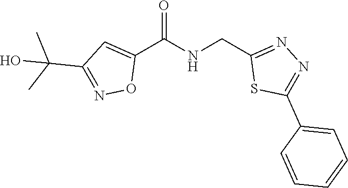 [SMILES: CC(C)(O)c1cc(C(=O)NCc2nnc(-c3ccccc3)s2)on1] | 3-(2-hydroxypropan-2-yl)-N-((5-phenyl-1,3,4- thiadiazol-2-yl)methyl)isoxazole-5-carboxamide |
| 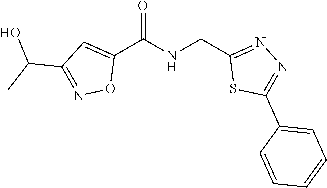 [SMILES: CC(O)c1cc(C(=O)NCc2nnc(-c3ccccc3)s2)on1] | 3-(1-hydroxyethyl)-N-((5-phenyl-1,3,4-thiadiazol- 2-yl)methyl)isoxazole-5-carboxamide |
| 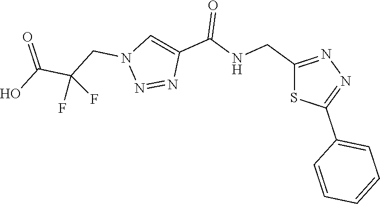 [SMILES: O=C(NCc1nnc(-c2ccccc2)s1)c1cn(CC(F)(F)C(=O)O)nn1] | 2,2-difluoro-3-(4-(((5-phenyl-1,3,4-thiadiazol-2- yl)methyl)carbamoyl)-1H-1,2,3-triazol-1- yl)propanoic acid |
| 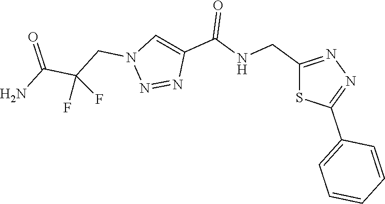 [SMILES: NC(=O)C(F)(F)Cn1cc(C(=O)NCc2nnc(-c3ccccc3)s2)nn1] | 1-(3-amino-2,2-difluoro-3-oxopropyl)-N-((5- phenyl-1,3,4-thiadiazol-2-yl)methyl)-1H-1,2,3- triazole-4-carboxamide |
| 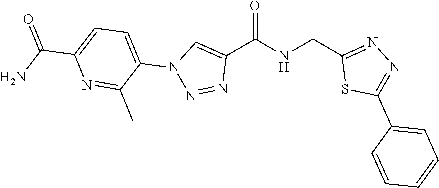 [SMILES: Cc1nc(C(N)=O)ccc1-n1cc(C(=O)NCc2nnc(-c3ccccc3)s2)nn1] | 6-methyl-5-(4-(((5-phenyl-1,3,4-thiadiazol-2- yl)methyl)carbamoyl)-1H-1,2,3-triazol-1- yl)picolinamide |
| 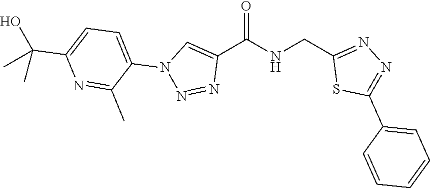 [SMILES: Cc1nc(C(C)(C)O)ccc1-n1cc(C(=O)NCc2nnc(-c3ccccc3)s2)nn1] | 1-(6-(2-hydroxypropan-2-yl)-2-methylpyridin-3- yl)-N-((5-phenyl-1,3,4-thiadiazol-2-yl)methyl)-1H- 1,2,3-triazole-4-carboxamide |
| 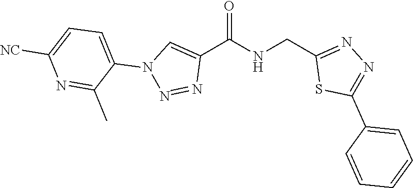 [SMILES: Cc1nc(C#N)ccc1-n1cc(C(=O)NCc2nnc(-c3ccccc3)s2)nn1] | 1-(6-cyano-2-methylpyridin-3-yl)-N-((5-phenyl- 1,3,4-thiadiazol-2-yl)methyl)-1H-1,2,3-triazole-4- carboxamide |
| 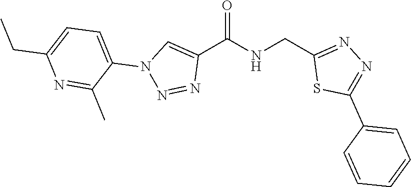 [SMILES: CCc1ccc(-n2cc(C(=O)NCc3nnc(-c4ccccc4)s3)nn2)c(C)n1] | 1-(6-ethyl-2-methylpyridin-3-yl)-N-((5-phenyl- 1,3,4-thiadiazol-2-yl)methyl)-1H-1,2,3-triazole-4- carboxamide |
| 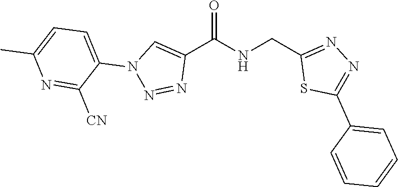 [SMILES: Cc1ccc(-n2cc(C(=O)NCc3nnc(-c4ccccc4)s3)nn2)c(C#N)n1] | 1-(2-cyano-6-methylpyridin-3-yl)-N-((5-phenyl- 1,3,4-thiadiazol-2-yl)methyl)-1H-1,2,3-triazole-4- carboxamide |
| 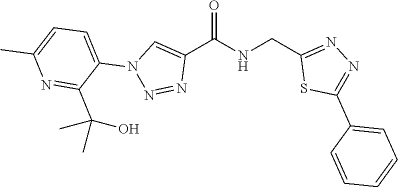 [SMILES: Cc1ccc(-n2cc(C(=O)NCc3nnc(-c4ccccc4)s3)nn2)c(C(C)(C)O)n1] | 1-(2-(2-hydroxypropan-2-yl)-6-methylpyridin-3- yl)-N-((5-phenyl-1,3,4-thiadiazol-2-yl)methyl)-1H- 1,2,3-triazole-4-carboxamide |
| 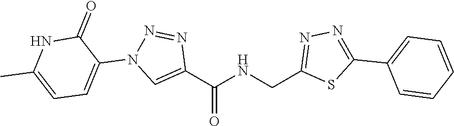 [SMILES: Cc1ccc(-n2cc(C(=O)NCc3nnc(-c4ccccc4)s3)nn2)c(=O)[nH]1] | 1-(6-methyl-2-oxo-1,2-dihydropyridin-3-yl)-N-((5- phenyl-1,3,4-thiadiazol-2-yl)methyl)-1H-1,2,3- triazole-4-carboxamide |
| 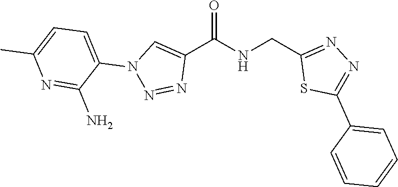 [SMILES: Cc1ccc(-n2cc(C(=O)NCc3nnc(-c4ccccc4)s3)nn2)c(N)n1] | 1-(2-amino-6-methylpyridin-3-yl)-N-((5-phenyl- 1,3,4-thiadiazol-2-yl)methyl)-1H-1,2,3-triazole-4- carboxamide |
| 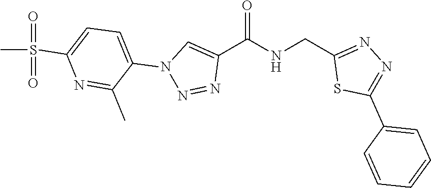 [SMILES: Cc1nc(S(C)(=O)=O)ccc1-n1cc(C(=O)NCc2nnc(-c3ccccc3)s2)nn1] | 1-(2-methyl-6-(methylsulfonyl)pyridin-3-yl)- N-((5-phenyl-1,3,4-thiadiazol-2-yl)methyl)-1H- 1,2,3-triazole-4-carboxamide |
| 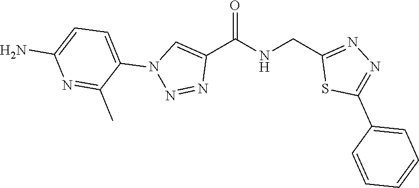 [SMILES: Cc1nc(N)ccc1-n1cc(C(=O)NCc2nnc(-c3ccccc3)s2)nn1] | 1-(6-amino-2-methylpyridin-3-yl)-N-((5-phenyl- 1,3,4-thiadiazol-2-yl)methyl)-1H-1,2,3-triazole-4- carboxamide |
| 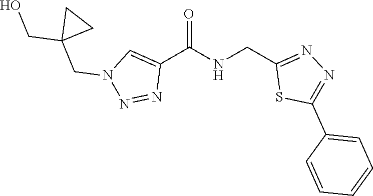 [SMILES: O=C(NCc1nnc(-c2ccccc2)s1)c1cn(CC2(CO)CC2)nn1] | 1-((1-(hydroxymethyl)cyclopropyl)methyl)-N-((5- phenyl-1,3,4-thiadiazol-2-yl)methyl)-1H-1,2,3- triazole-4-carboxamide |
| 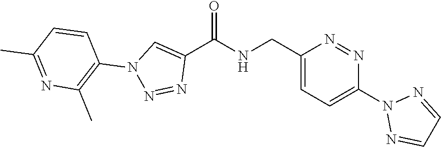 [SMILES: Cc1ccc(-n2cc(C(=O)NCc3ccc(-n4nccn4)nn3)nn2)c(C)n1] | N-((6-(2H-1,2,3-triazol-2-yl)pyridazin-3- yl)methyl)-1-(2,6-dimethylpyridin-3-yl)-1H-1,2,3- triazole-4-carboxamide |
| 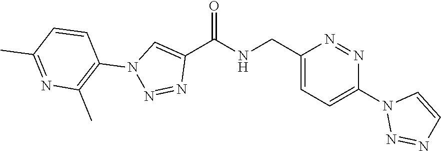 [SMILES: Cc1ccc(-n2cc(C(=O)NCc3ccc(-n4ccnn4)nn3)nn2)c(C)n1] | N-((6-(1H-1,2,3-triazol-1-yl)pyridazin-3- yl)methyl)-1-(2,6-dimethylpyridin-3-yl)-1H-1,2,3- triazole-4-carboxamide |
| 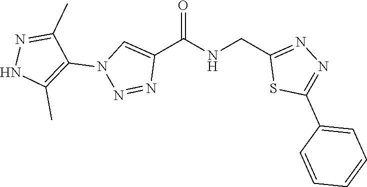 [SMILES: Cc1n[nH]c(C)c1-n1cc(C(=O)NCc2nnc(-c3ccccc3)s2)nn1] | 1-(3,5-dimethyl-1H-pyrazol-4-yl)-N-((5-phenyl- 1,3,4-thiadiazol-2-yl)methyl)-1H-1,2,3-triazole-4- carboxamide |
| 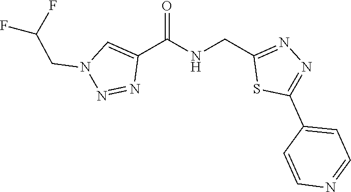 [SMILES: O=C(NCc1nnc(-c2ccncc2)s1)c1cn(CC(F)F)nn1] | 1-(2,2-difluoroethyl)-N-((5-(pyridin-4-yl)-1,3,4- thiadiazol-2-yl)methyl)-1H-1,2,3-triazole-4- carboxamide |
| 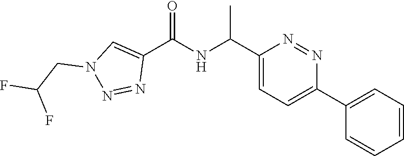 [SMILES: CC(NC(=O)c1cn(CC(F)F)nn1)c1ccc(-c2ccccc2)nn1] | 1-(2,2-difluoroethyl)-N-(1-(6-phenylpyridazin-3- yl)ethyl)-1H-1,2,3-triazole-4-carboxamide |

and pharmaceutically acceptable salts thereof.

### Definitions

The term “aryl” means a monocyclic, bicyclic or tricyclic carbocyclic aromatic ring or ring system containing 5-14 carbon atoms, wherein at least one of the rings is aromatic. Examples of aryl include phenyl and naphthyl.

The term “alkylene,” or “alkylenyl” by itself or as part of another substituent means a divalent straight or branched chain hydrocarbon radical having the stated number of carbon atoms. For example, —(C₁-C₅) alkylenyl, would include, e.g., —CH₂—, —CH₂CH₂—, —CH₂CH₂CH₂—, —CH₂CH₂CH₂CH₂—, —CH₂CH(CH₃)CH₂— or —CH₂CH₂CH₂CH₂CH₂—.

The term “halogen” includes a fluorine, a chlorine, a bromine or an iodine radical.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “C₃-C₆cycloalkyl” encompasses bridged, saturated or unsaturated cycloalkyl groups having 3 to 6 carbons. Examples of cycloalkyl include cyclopropyl, cyclobutyl, cyclopentyl and cyclohexyl.

The term “C₃-C₁₀cycloalkyl” encompasses bridged, saturated or unsaturated cycloalkyl groups having 3 to 10 carbons. “Cycloalkyl” also includes non-aromatic rings as well as monocyclic, non-aromatic rings fused to a saturated cycloalkyl group and aromatic rings fused to a saturated cycloalkyl group. Examples of cycloalkyl include cyclopropyl, cyclobutyl, cyclopentyl, cyclohexyl, cycloheptyl and the like. Examples described by structure include:

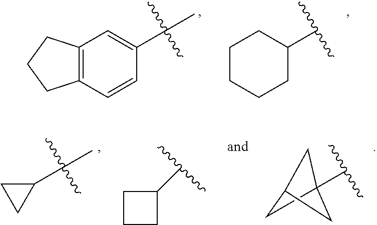

The term “heteroaryl” means an aromatic cycloheteroalkyl that contains at least one ring heteroatom selected from O, S and N. Examples of heteroaryl groups include pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, isoquinolyl, and the like. A “five or six-membered nitrogen-containing heteroaryl ring” means a heteroaryl with five or six ring atoms, wherein at least one ring atom is nitrogen.

The term “cycloheteroalkyl” means mono- or bicyclic or bridged partially unsaturated or saturated rings containing at least one heteroatom selected from N, S and O, each of said rings having from 3 to 10 atoms in which the point of attachment may be carbon or nitrogen. Examples include tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and tetrahydropyran. The term also includes partially unsaturated monocyclic rings that are not aromatic, such as 2- or 4-pyridones attached through the nitrogen or N-substituted-(1H,3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. Examples described by structure include:

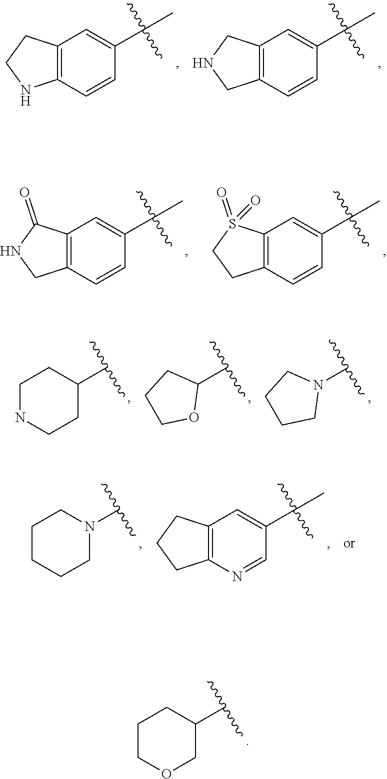

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, including a human, canine, feline, bovine, or porcine patient, preferably a human patient, receiving or about to receive medical treatment.

The compounds of the invention may contain one or more asymmetric centers and can thus occur as racemates, racemic mixtures, single enantiomers, diastereomeric mixtures, and individual diastereomers. The invention is meant to comprehend all such isomeric forms of these compounds.

Some of the compounds described herein contain olefinic double bonds, and unless specified otherwise, are meant to include both E and Z geometric isomers.

Some of the compounds described herein contain substituted cycloalkanes having cis- and trans-isomers, and unless specified otherwise, are meant to include both cis- and trans-geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that the invention is meant to include the pharmaceutically acceptable salts, and also salts that are not pharmaceutically acceptable, of the compounds described herein, when they are used as precursors to the free compounds or their pharmaceutically acceptable salts or in other synthetic manipulations.

Solvates, and in particular, the hydrates of the compounds of the structural formulas described herein are included in the invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples.

Isotopically-enriched compounds can be prepared without undue experimentation by conventional techniques well known to those skilled in the art or by processes analogous to those described in the Schemes and Examples herein using appropriate isotopically-enriched reagents or Intermediates.

A wavy line [symbol] as used herein, indicates a point of attachment to the rest of the compound.

A line drawn into a ring, for example:

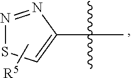

indicates that the bond may be attached to any of the substitutable ring atoms.

Unless expressly stated to the contrary in a particular context, any of the various cyclic ring and ring system variables or substituents described herein may be attached to the rest of the compound at any ring atom (i.e., any carbon atom or any heteroatom) provided that a stable compound results.

It should be noted that chemically unstable compounds are excluded from the embodiments contained herein.

Unless expressly stated to the contrary, all ranges cited herein are inclusive. For example, a heteroaryl described as containing from “one to three heteroatoms” means the ring can contain one, two, three or four heteroatoms. It is also to be understood that any range cited herein includes within its scope all of the sub-ranges within that range. Thus, for example, a heterocyclic ring described as containing from “one to four heteroatoms” is intended to include as aspects thereof, heterocyclic rings containing two to four heteroatoms, three to four heteroatoms, one to three heteroatoms, two or three heteroatoms, one or two heteroatoms, one heteroatom, two heteroatoms, three heteroatoms, and four heteroatoms. Similarly, C₁-C₆ when used with a chain, for example an alkyl chain, means that the chain can contain one, two, three, four, five and six carbon atoms. It also includes all ranges contained therein including C₁-C₅, C₁-C₄, C₁-C₃, C₁-C₂, C₂-C₆, C₃-C₆, C₄-C₆, C₅-C₆, and all other possible combinations.

### Methods of Treatment

Also encompassed by the invention are methods of preventing, treating or ameliorating IL4I1-related diseases. The compounds described herein can be effective in preventing, treating or ameliorating various IL4I1-related diseases, such as cancer. Described herein are methods for treatment of cancer displaying IL4I1-expressing cells in a patient. Described herein are methods for prevention of cancer displaying IL4I1-expressing cells in a patient. Described herein are methods for ameliorating of cancer displaying IL4I1-expressing cells in a patient.

In one embodiment described herein, the cancer to be treated is selected from the group consisting of cancers displaying IL4I1-expressing cells and lymphomas displaying IL411-expressing cells. In certain embodiment, the cancers to be treated are solid tumors. In certain embodiments, the cancers to be treated are selected from carcinomas, sarcomas, mesotheliomas, blastomas and germ cell tumors. In another particular embodiment, cancers to be treated are selected from the group consisting of mesotheliomas, non-small-cell lung carcinomas, colon carcinoma, breast carcinoma, thyroid carcinoma, testicular germ cell tumors and ovarian carcinoma, displaying IL4I1-expressing cells.

In another specific embodiment, the cancer to be treated is a lymphoma displaying IL4I1-expressing cells typically selected from B-cell lymphomas displaying IL4I1-expressing cells.

In certain embodiments, the cancer to be treated is selected from the group consisting of PMBL (Primary Mediastinal large B-cell Lymphoma), classical Hodgkin lymphoma (cHL), NLPHL (Nodular lymphocyte predominant Hodgkin lymphoma), non-mediastinal Diffuse Large B-Cell Lymphoma (DLBCL) and SLL/CLL (Small Lymphocytic Lymphoma/Chronic Lymphocytic Leukemia), displaying IL4I1-expressing cells. In another specific embodiment, the cancer to be treated is a lymphoma displaying IL4I1-expressing cells.

In one embodiment described herein, the cancer to be prevented is selected from the group consisting of cancers displaying IL4I1-expressing cells and lymphomas displaying IL4I1-expressing cells. In certain embodiment, the cancers to be prevented are solid tumors. In certain embodiments, the cancers to be prevented are typically selected from carcinomas, sarcomas, mesotheliomas, blastomas and germ cell tumors. In another particular embodiment, cancers to be prevented are typically selected from the group consisting of mesotheliomas, non-small-cell lung carcinomas, colon carcinoma, breast carcinoma, thyroid carcinoma, testicular germ cell tumors and ovarian carcinoma, displaying IL4I1-expressing cells.

In another specific embodiment, the cancer to be prevented is a lymphoma displaying IL4I1-expressing cells typically selected from B-cell lymphomas displaying IL4I1-expressing cells.

In certain embodiments, the cancer to be prevented is selected from the group consisting of PMBL (Primary Mediastinal large B-cell Lymphoma), classical Hodgkin lymphomas (cHL), NLPHL (Nodular lymphocyte predominant Hodgkin lymphoma), non-mediastinal Diffuse Large B-Cell Lymphoma (DLBCL), large B-cell lymphoma (DLBCL), acute myeloid leukemia (AML) and SLL/CLL (Small Lymphocytic Lymphoma/Chronic Lymphocytic Leukemia), displaying IL4I1-expressing cells. In another specific embodiment, the cancer to be treated is a lymphoma displaying IL4I1-expressing cells.

In one embodiment described herein, the cancer to be ameliorated is selected from the group consisting of cancers displaying IL4I1-expressing cells and lymphomas displaying IL4I1-expressing cells. In certain embodiments, the cancers to be ameliorated are typically selected from carcinomas, sarcomas, mesotheliomas, blastomas and germ cell tumors. In another particular embodiment, cancers to be ameliorated are typically selected from the group consisting of mesotheliomas, non-small-cell lung carcinomas, colon carcinoma, breast carcinoma, thyroid carcinoma, testicular germ cell tumors and ovarian carcinoma, displaying IL4I1-expressing cells.

In another specific embodiment, the cancer to be ameliorated is a lymphoma displaying IL4I1-expressing cells typically selected from B-cell lymphomas displaying IL4I1-expressing cells.

In certain embodiments, the cancer to be ameliorated is selected from the group consisting of PMBL (Primary Mediastinal large B-cell Lymphoma), classical Hodgkin lymphomas (cHL), NLPHL (Nodular lymphocyte predominant Hodgkin lymphoma), non-mediastinal Diffuse Large B-Cell Lymphoma (DLBCL) and SLL/CLL (Small Lymphocytic Lymphoma/Chronic Lymphocytic Leukemia), displaying IL4I1-expressing cells. In another specific embodiment, the cancer to be ameliorated is a lymphoma displaying IL4I1-expressing cells

### Pharmaceutical Compositions

Compounds described herein may be administered to a patient orally or parenterally. As formulated into a dosage form suitable for administration, the compounds described herein can be used as a pharmaceutical composition for the prevention, treatment, or remedy of the above diseases.

In clinical use of the compounds described herein, usually, the compound is formulated into various preparations together with pharmaceutically acceptable additives according to the dosage form, and may then be administered. By “pharmaceutically acceptable” it is meant the additive, carrier, diluent or excipient must be compatible with the other ingredients of the formulation and not deleterious to the recipient thereof. As such, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders and suppositories; and liquid preparations such as syrups, elixirs and injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. In specific embodiments, the dose is from about 0.01 to about 25 mg/kg/day, in particular embodiments, from about 0.05 to about 10 mg/kg/day, or from about 0.001 to about 50 mg/kg/day. For oral administration, the compositions are preferably provided in the form of tablets or capsules containing from 0.01 mg to 1,000 mg. In specific embodiments, the dose is 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 or 1,000 milligrams of a compound described herein. This dosage regimen may be adjusted to provide the optimal therapeutic response.

### Combination Therapy

The compounds of the invention are further useful in methods for the prevention or treatment of the aforementioned diseases, disorders and conditions in combination with other therapeutic agents.

The compounds of the invention may be used in combination with one or more other drugs in the treatment, prevention, suppression or amelioration of diseases or conditions for which compounds described herein or the other drugs may have utility, where the combination of the drugs together are safer or more effective than either drug alone. Such other drug(s) may be administered in an amount commonly used therefore, contemporaneously or sequentially with a compound described herein or a pharmaceutically acceptable salt thereof. When a compound described herein is used contemporaneously with one or more other drugs, the pharmaceutical composition may in specific embodiments contain such other drugs and the compound described herein or its pharmaceutically acceptable salt in unit dosage form. However, the combination therapy may also include therapies in which the compound described herein or its pharmaceutically acceptable salt and one or more other drugs are administered on different overlapping schedules. It is also contemplated that when used in combination with one or more other active ingredients, the compounds of the invention and the other active ingredients may be used in lower doses than when each is used singly. Accordingly, the pharmaceutical compositions of the invention include those that contain one or more other active ingredients, in addition to a compound described herein or a pharmaceutically acceptable salt thereof.

Examples of other active ingredients that may be administered in combination with a compound of any of the Formulas described herein or a pharmaceutically acceptable salt thereof and either administered separately or in the same pharmaceutical composition, include, but are not limited to pain relieving agents, anti-angiogenic agents, anti-neoplastic agents, anti-diabetic agents, anti-infective agents, or gastrointestinal agents, or combinations thereof.

Suitable compounds that may be used in combination with a compound according to the invention include without limitation sildenafil, vardenafil, tadalafil and alprostadil, epoprostenol, iloprost, bosentan, amlodipine, diltiazem, nifedipine, ambrisentan and warfarin, fluticasone, budesonide, mometasone, flunisolide, beclomethasone, montelukast, zafirlukast, zileuton, salmeterol, formoterol, theophylline, albuterol, levalbuterol, pirbuterol, ipratropium, prednisone, methylprednisolone, omalizumab, corticosteroid and cromolyn, atorvastatin, lovastatin, simvastatin, pravastatin, fluvastatin, rosuvastatin, gemfibrozil, fenofibrate, nicotinic acid, clopidogrel and pharmaceutically acceptable salts thereof.

Additionally, a compound of any of the Formulas disclosed herein may be used in combination with one or more other active agents, including but not limited to, other anti-cancer agents that are used in the prevention, treatment, control, amelioration, or reduction of risk of a particular disease or condition (e.g., cell proliferation disorders). In one embodiment, a compound disclosed herein is combined with one or more other anti-cancer agents for use in the prevention, treatment, control amelioration, or reduction of risk of a particular disease or condition for which the compounds disclosed herein are useful. Such other active agents may be administered, by a route and in an amount commonly used therefor, contemporaneously or sequentially with a compound of the invention.

In one embodiment, the other active agent is selected from the group consisting of vascular endothelial growth factor (VEGF) receptor inhibitors, topoisomerase II inhibitors, smoothen inhibitors, alkylating agents, anti-tumor antibiotics, anti-metabolites, retinoids, immunomodulatory agents including but not limited to anti-cancer vaccines, CTLA-4, LAG-3 and PD-1 antagonists.

PD-1 is recognized as having an important role in immune regulation and the maintenance of peripheral tolerance. PD-1 is moderately expressed on naive T-cells, B-cells and NKT-cells and up-regulated by T-cell and B-cell receptor signaling on lymphocytes, monocytes and myeloid cells (Sharpe et al., Nature Immunology (2007); 8:239-245).

Two known ligands for PD-1, PD-L1 (B7-H1) and PD-L2 (B7-DC) are expressed in human cancers arising in various tissues. In large sample sets of, for example, ovarian, renal, colorectal, pancreatic, and liver cancers, and in melanoma, it was shown that PD-L1 expression correlated with poor prognosis and reduced overall survival irrespective of subsequent treatment. (Dong et al., Nat Med. 8(8):793-800 (2002); Yang et al., Invest Ophthamol Vis Sci. 49: 2518-2525 (2008); Ghebeh et al., Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953; Inman et al., Cancer 109: 1499-1505 (2007); Shimauchi et al., Int. J. Cancer 121:2585-2590 (2007); Gao et al., Clin. Cancer Research 15: 971-979 (2009); Nakanishi J., Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T-cells in breast cancer and melanoma (Ghebeh et al., BMC Cancer. 2008 8:5714-15 (2008); and Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T-cells to attenuate T-cell activation and to evade immune surveillance, thereby contributing to an impaired immune response against the tumor.

Immune checkpoint therapies targeting the PD-1 axis have resulted in groundbreaking improvements in clinical response in multiple human cancers (Brahmer, et al., N Engl J Med 2012, 366: 2455-65; Garon et al., N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; and Wolchok et al., N Engl J Med 2013, 369: 122-33).

“PD-1 antagonist” means any chemical compound or biological molecule that blocks binding of PD-L1 expressed on a cancer cell to PD-1 expressed on an immune cell (T-cell, B-cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCDILI, PDL1, B7H1, B7-4, CD274 and B7-H for PD-L1; and PDCD1L2, PDL2, B7-DC, Btdc and CD273 for PD-L2. In any of the treatment methods, medicaments and uses of the invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP_054862 and NP_079515, respectively.

PD-1 antagonists useful in any of the treatment methods, medicaments and uses of the invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)₂, scFv and Fv fragments. Examples of PD-1 antagonists include, but are not limited to, pembrolizumab (KEYTRUDA®, Merck and Co., Inc., Rahway, NJ, USA). “Pembrolizumab” (formerly known as MK-3475, SCH 900475 and lambrolizumab and sometimes referred to as “pembro”) is a humanized IgG4 mAb with the structure described in WHO Drug Information, Vol. 27, No. 2, pages 161-162 (2013). Additional examples of PD-1 antagonists include nivolumab (OPDIVO®, Bristol-Myers Squibb Company, Princeton, NJ, USA), atezolizumab (MPDL3280A; TECENTRIQ®, Genentech, San Francisco, CA, USA), durvalumab (IMFINZI®, Astra Zeneca Pharmaceuticals, LP, Wilmington, DE), avelumab (BAVENCIO®, Merck KGaA, Darmstadt, Germany and Pfizer, Inc., New York, NY), cemiplimab (LIBTAYO®, Regeneron Pharmaceuticals, Inc., Tarrytown, NY, and Sanofi-Aventis LLC, Bridgewater, NJ, U.S.), and dostarlimab (JEMPERLI®, GlaxoSmithKline LLC, Philadelphia, PA).

Examples of monoclonal antibodies (mAbs) that bind to human PD-1, and useful in the treatment methods, medicaments and uses of the invention, are described in U.S. Pat. Nos. 7,488,802, 7,521,051, 8,008,449, 8,354,509, 8,168,757, WO2004/004771, WO2004/072286, WO2004/056875, and US2011/0271358.

Examples of mAbs that bind to human PD-L1, and useful in the treatment methods, medicaments and uses of the invention, are described in WO2013/019906, WO2010/077634 A1 and U.S. Pat. No. 8,383,796. Specific anti-human PD-L1 mAbs useful as the PD-1 antagonist in the treatment methods, medicaments and uses of the invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in any of the treatment methods, medicaments and uses of the invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010/027827 and WO2011/066342. Specific fusion proteins useful as the PD-1 antagonist in the treatment methods, medicaments and uses of the invention include AMP-224 (also known as B7-DCIg), which is a PD-L2-FC fusion protein that binds to human PD-1.

Thus, one embodiment provides a method of treating cancer comprising administering an effective amount of a compound of the invention, or a pharmaceutically acceptable salt thereof, in combination with a PD-1 antagonist to a subject in need thereof. In such embodiments, the compounds of the invention, or a pharmaceutically acceptable salt thereof, and the PD-1 antagonist are administered concurrently or sequentially.

Specific non-limiting examples of such cancers in accordance with this embodiment include melanoma (including unresectable or metastatic melanoma), head & neck cancer (including recurrent or metastatic head and neck squamous cell cancer (HNSCC)), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, and salivary cancer.

In one embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from unresectable or metastatic melanoma, melanoma following complete resection, recurrent, metastatic, or unresectable head and neck squamous cell cancer (HNSCC), classical Hodgkin lymphoma (cHL), urothelial carcinoma, gastric cancer, Merkel cell carcinoma, renal cell carcinoma, endometrial carcinoma, tumor mutational burden-high (TMB-H) cancer, cervical cancer, primary mediastinal large-B-cell lymphoma, microsatellite instability-high (MSI-H) or mismatch repair deficient cancer, non-small cell lung cancer, esophageal cancer, cutaneous squamous cell carcinoma, triple negative breast cancer, and hepatocellular carcinoma. In one such embodiment, the agent is a PD-1 antagonist. In one such embodiment, the agent is pembrolizumab. In another such embodiment, the agent is nivolumab. In another such embodiment, the agent is atezolizumab. In other such embodiments, the agent is durvalumab or avelumab. In one embodiment, the agent is cemiplimab. In one embodiment, the agent is dostarlimab.

Pembrolizumab is approved by the U.S. FDA for the treatment of patients with unresectable or metastatic melanoma and for the adjuvant treatment of melanoma following complete resection, and for the treatment of certain patients with recurrent, metastatic, or unresectable head and neck squamous cell cancer (HNSCC), classical Hodgkin lymphoma (cHL), urothelial carcinoma, gastric cancer, Merkel cell carcinoma, renal cell cancer, endometrial cancer, tumor mutational burden-high (TMB-H) cancer, cervical cancer, primary mediastinal large-B-cell lymphoma, microsatellite instability-high (MSI-H) or mismatch repair deficient cancer, non-small cell lung cancer, esophageal cancer, cutaneous squamous cell carcinoma, triple negative breast cancer, and hepatocellular carcinoma, as described in the Prescribing Information for KEYTRUDA™ (Merck & Co., Inc., Rahway, NJ USA; initial U.S. approval 2014, updated January 2023). In another embodiment, there is provided a method of treating cancer comprising administering an effective amount of a compound of the invention, or a pharmaceutically acceptable salt thereof, in combination with pembrolizumab to a person in need thereof, wherein said cancer is selected from unresectable or metastatic melanoma, adjuvant melanoma, recurrent, unresectable or metastatic head and neck squamous cell cancer (HNSCC), classical Hodgkin lymphoma (cHL), urothelial carcinoma, gastric cancer, Merkel cell carcinoma, renal cell cancer, endometrial cancer, tumor mutational burden-high (TMB-H) cancer, cervical cancer, primary mediastinal large-B-cell lymphoma, microsatellite instability-high (MSI-H) or mismatch repair deficient cancer, non-small cell lung cancer, esophageal cancer, cutaneous squamous cell carcinoma, triple negative breast cancer, and hepatocellular carcinoma.

In another embodiment, there is provided a method of treating cancer comprising administering an effective amount of a compound of the invention, or a pharmaceutically acceptable salt thereof, in combination with a PD-1 antagonist to a person in need thereof, wherein said cancer is selected from melanoma, non-small cell lung cancer, head and neck squamous cell cancer (HNSCC), Hodgkin lymphoma, primary mediastinal large B-cell lymphoma, urothelial carcinoma, microsatellite instability-high cancer, gastric cancer, Merkel cell carcinoma, hepatocellular carcinoma, esophageal cancer and cervical cancer. In one such embodiment, the agent is a PD-1 antagonist. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In other such embodiment, the agent is durvalumab or avelumab.

In another embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from melanoma, non-small cell lung cancer, small cell lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In other such embodiment, the agent is durvalumab or avelumab.

In one embodiment, there is provided a method of treating unresectable or metastatic melanoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating recurrent or metastatic head and neck squamous cell cancer (HNSCC) comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating classical Hodgkin lymphoma (cHL) comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating urothelial carcinoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating gastric cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating cervical cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating primary mediastinal large-B-cell lymphoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating microsatellite instability-high (MSI-H) cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating non-small cell lung cancer comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

In one embodiment, there is provided a method of treating hepatocellular carcinoma comprising administering an effective amount of a compound of the invention, or a pharmaceutically acceptable salt thereof, to a person in need thereof, in combination with a PD-1 antagonist. In one such embodiment, the agent is pembrolizumab. In another such embodiment, the agent is nivolumab. In another such embodiment, the agent is atezolizumab. In other such embodiment, the agent is durvalumab or avelumab.

Examples of vascular endothelial growth factor (VEGF) receptor inhibitors include, but are not limited to, bevacizumab (sold under the trademark AVASTIN by Genentech/Roche), axitinib, (N-methyl-2-[[3-[([pound])-2-pyridin-2-ylethenyl]-1H-indazol-6-yl]sulfanyl]benzamide, also known as AG013736, and described in PCT Publication No. WO01/002369), Brivanib Alaninate ((S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, also known as BMS-582664), motesanib (N-(2,3-dihydro-3,3-dimethyl-1H-indol-6-yl)-2-[(4-pyridinyimethy)amino]-3-pyridinecarboxamide. and described in PCT Publication No. WO 02/068470), pasireotide (also known as SO 230, and described in PCT Publication No. WO02/010192), and sorafenib (sold under the tradename NEXAVAR).

Examples of topoisomerase II inhibitors include but are not limited to, etoposide (also known as VP-16 and Etoposide phosphate, sold under the tradenames TOPOSAR, VEPESID and ETOPOPHOS), and teniposide (also known as VM-26, sold under the tradename VUMON).

Examples of alkylating agents include but are not limited to, 5-azacytidine (sold under the trade name VIDAZA), decitabine (sold under the trade name of DECOGEN), temozolomide (sold under the trade names TEMODAR and TEMODAL by Schering-Plough/Merck), dactinomycin (also known as actinomycin-D and sold under the tradename COSMEGEN), melphalan (also known as L-PAM, L-sarcolysin, and phenylalanine mustard, sold under the tradename ALKERAN), altretamine (also known as hexamethylmelamine (HMM), sold under the tradename HEXALEN), carmustine (sold under the tradename BCNU), bendamustine (sold under the tradename TREANDA), busulfan (sold under the tradenames BUSULFEX and MYLERAN), carboplatin (sold under the tradename PARAPLATIN), lomustine (also known as CCNU, sold under the tradename CeeNU), cisplatin (also known as CDDP, sold under the tradenames PLATINOL and PLATINOL-AQ), chlorambucil (sold under the tradename LEUKERAN), cyclophosphamide (sold under the tradenames CYTOXAN and NEOSAR), dacarbazine (also known as DTIC, DIC and imidazole carboxamide, sold under the tradename DTIC-DOME), altretamine (also known as hexamethylmelamine (HMM) sold under the tradename HEXALEN), ifosfamide (sold under the tradename IFEX), procarbazine (sold under the tradename MATULANE), mechlorethamine (also known as nitrogen mustard, mustine and mechloroethamine hydrochloride, sold under the tradename MUSTARGEN), streptozocin (sold under the tradename ZANOSAR), thiotepa (also known as thiophosphoamide, TESPA and TSPA, and sold under the tradename THIOPLEX).

Examples of anti-tumor antibiotics include, but are not limited to, doxorubicin (sold under the tradenames ADRIAMYCIN and RUB EX), bleomycin (sold under the tradename LENOXANE), daunorubicin (also known as dauorubicin hydrochloride, daunomycin, and rubidomycin hydrochloride, sold under the tradename CERUBIDINE), daunorubicin liposomal (daunorubicin citrate liposome, sold under the tradename DAUNOXOME), mitoxantrone (also known as DHAD, sold under the tradename NOVANTRONE), epirubicin (sold under the tradename ELLENCE), idarubicin (sold under the tradenames IDAMYCIN, IDAMYCIN PFS), and mitomycin C (sold under the tradename MUTAMYCIN).

Examples of anti-metabolites include, but are not limited to, claribine (2-chlorodeoxyadenosine, sold under the tradename LEUSTATIN), 5-fluorouracil (sold under the tradename ADRUCIL), 6-thioguanine (sold under the tradename PURINETHOL), pemetrexed (sold under the tradename ALIMTA), cytarabine (also known as arabinosylcytosine (Ara-C), sold under the tradename CYTOSAR-U), cytarabine liposomal (also known as Liposomal Ara-C, sold under the tradename DEPOCYT), decitabine (sold under the tradename DACOGEN), hydroxyurea (sold under the tradenames HYDREA, DROXIA and MYLOCEL), fludarabine (sold under the tradename FLUDARA), floxuridine (sold under the tradename FUDR), cladribine (also known as 2-chlorodeoxyadenosine (2-CdA) sold under the tradename LEUSTATIN), methotrexate (also known as amethopterin, methotrexate sodium (MTX), sold under the tradenames RHEUMATREX and TREXALL), and pentostatin (sold under the tradename NIPENT).

Examples of retinoids include, but are not limited to, alitretinoin (sold under the tradename PANRETIN), tretinoin (all-trans retinoic acid, also known as ATRA, sold under the tradename VESANOID), Isotretinoin (13-c/s-retinoic acid, sold under the tradenames ACCUTANE, AMNESTEEM, CLARAVIS, CLARUS, DECUTAN, ISOTANE, IZOTECH, ORATANE, ISOTRET, and SOTRET), and bexarotene (sold under the tradename TARGRETIN).

In such combinations a compound of the invention and other active agents may be administered separately or in conjunction. In addition, the administration of one element may be prior to, concurrent to, or subsequent to the administration of other agent(s).

### EXAMPLES

The meanings of the abbreviations in Examples are shown below.
ACN=CH₃CN=MeCN=Acetonitrile
Ac=acetyl
AcOH=acetic acid
AIBN=azobisisobutyronitrile
APhos-Pd-G3=Palladium G3-(4-(N,N-Dimethylamino)phenyl)di-tert-butylphosphine=[4-(Di-tert-butylphosphino)-N,N-dimethylaniline-2-(2′-aminobiphenyl)]palladium(II) methanesulfonate
APhos-Pd-G4=4-Ditert-butylphosphanyl-N,N-dimethylaniline; methanesulfonic acid; N-methyl-2-phenylaniline; palladium
Boc=tert-butyloxycarbonyl
Boc-Ser(Bzl)-OH=N-(tert-Butoxycarbonyl)-O-benzyl-L-serine
BPO=benzoyl peroxide
cataCXium A®=Di(1-adamantyl)-N-butylphosphine
CDI=1,1′-carbonyldiimidazole
CELITE=diatomaceous earth
CF₃CH₂OH=2,2,2-trifluoroethanol
Conc.=concentrated
CO₂=carbon dioxide
Cs₂CO₃=cesium carbonate
DCE=dichloroethane
DCM=dichloromethane
DEA=diethylamine
DIEA=DIPEA=N,N-diisopropylethylamine=Hünig's base
DMA=Dimethylacetamide
DMAP=4-Dimethylaminopyridine
DMF=N,N-dimethylformamide
DMSO=dimethyl sulfoxide
DPPA=Diphenylphosphoryl azide
DPPE=1,2-bis(diphenylphosphino)ethane
EDCI=EDC=1-ethyl-3-(3-dimethylaminopropyl)carbodiimide
EtOAc=ethyl acetate
h=hours
H₂=hydrogen
H₂O=water
HATU=1-[bis(dimethylamino)methylene]-1H-1,2,3-triazolo[4,5-b]pyridinium 3-oxid
hexafluorophosphate
HBr=hydrogen bromide
HCl=hydrochloric acid
HFBA=heptafluorobutyric acid
HOBT=hydroxybenzotriazole
K₂CO₃=potassium carbonate
K₃PO₄=tripotassium phosphate
LCMS=liquid chromatography-mass spectrometry
LHMDS=LiHMDS=lithium bis(trimethylsilyl)amide
LiAlH₄=lithium aluminum hydride
LG=leaving group
LiF=lithium fluoride
LiOH=lithium hydroxide
MBTE=methyl tert-butyl ether
min=minutes
MeOH=methanol
MgSO₄=magnesium sulfate
Ms=mesyl
NaBH₄=sodium borohydride
NaCl=sodium chloride
NaHCO₃=sodium bicarbonate
NaOH=sodium hydroxide
Na₂SO₄=sodium sulfate
NaH=sodium hydride
NBS=N-Bromosuccinimide
NCS=N-Chlorosuccinimide
NH₄Cl=ammonium chloride
NH₄OH=ammonium hydroxide
OMs=mesylate
OTs=tosylate
OTf=triflates
Pd(OH)₂/C=Pearlman's catalysts=palladium hydroxide on carbon
Pd₂(dba)₃=Tris(dibenzylideneacetone)dipalladium(0)
Pd(dppf)Cl₂=[1,1′-Bis(diphenylphosphino)ferrocene]palladium(II) dichloride
Pd (dtbpf) Cl₂=[1,1′-Bis(di-tert-butylphosphino)ferrocene]dichloropalladium(II)
rpm=revolutions per minute
RuPhos Pd G3=(2-Dicyclohexylphosphino-2′,6′-diisopropoxy-1,1′-biphenyl)[2-(2′-amino-1,1′-biphenyl)]palladium(II) methanesulfonate=RuPhos-G3-Palladacycle
SFC=supercritical fluid chromatography
sSPhos Pd G2=chloro(sodium-2-dicyclohexylphosphino-2′,6′-dimethoxy-1,1′-biphenyl-3′-sulfonate)[2-(2′-amino-1,1′-biphenyl)]palladium(II)
TBD=3,4,6,7,8,9-hexahydro-2H-pyrido[1,2-a]pyrimidine
t-BuOH=tert-butyl alcohol
TCCA=Trichloroisocyanuric acid
TEA=triethylamine
Tf=triflate
TFA=trifluoroacetic acid
TFAA=Trifluoroacetic anhydride
THF=tetrahydrofuran
TLC=Thin Layer Chromatography
TMEDA: tetramethylethylenediamine
Ts=tosyl
Xphos-Pd G2=Chloro(2-dicyclohexylphosphino-2′,4′,6′-triisopropyl-1,1′-biphenyl)[2-(2′-amino-1,1′-biphenyl)]palladium(II)=X-Phos aminobiphenyl palladium chloride precatalyst 1 Standard atmosphere [atm]=101325 pascal [Pa]=14.6959488 psi

The meanings of the abbreviations in the nuclear magnetic resonance spectra are shown below:

s=singlet, d=doublet, dd=double doublet, dt=double triplet, ddd=double double doublet, Sept=septet, t=triplet, m=multiplet, br=broad, brs=broad singlet, q=quartet J=coupling constant and Hz=hertz.

Compounds of this invention can be prepared using the intermediates and processes outlined below. The various starting materials used are commercially available or are readily made by a person skilled in the art. The following exemplified compounds of the invention may contain one or more asymmetric centers and can thus occur as racemates, racemic mixtures, single enantiomers, diastereomeric mixtures, and individual diastereomers. The invention is meant to comprehend all such isomeric forms of these compounds. In the examples, where a compound has one or more stereocenters, the stereocenters are indicated with an asterisk, as shown below:

### Schemes

In general, intermediates 1.3 can be synthesized according to scheme 1.

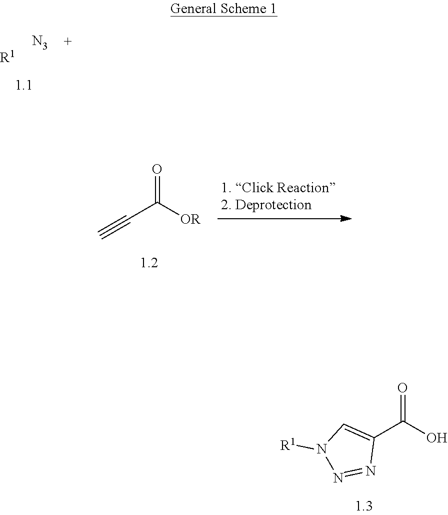

There are several methods known to one skilled in the art to form azides 1.1 through substitution reactions of alkyl or heteroaryl halides or pseudohalides and a source of azide ion. Alternatively, the substrate could activated in situ to form a suitable leaving group that then undergoes substitution with an azide ion. Azides 1.1 can also be obtained from amines using an appropriate diazo transfer reagent. These azides can be isolated or preferably used in crude form without isolation. The azides can engage in a copper catalyzed “click reaction” with suitably substituted alkynoates 1.2. The protected intermediate triazoles are then hydrolyzed using acidic or basic conditions to afford the triazole carboxylic acid intermediates 1.3.

In general, compounds of Formula I were synthesized according to one of Schemes 2-5

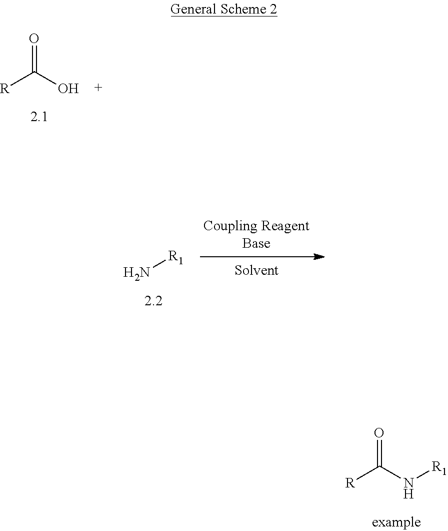

Certain compounds of Formula I were synthesized by reacting the appropriate carboxylic acid 2.1 with a primary amine 2.2 using an appropriate coupling reagent, base, solvent, time and temperature.

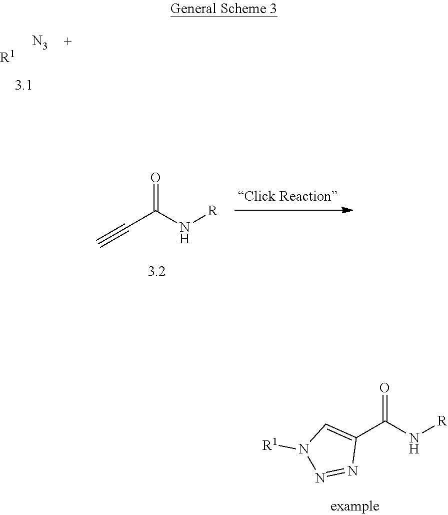

Certain compounds of Formula I were synthesized by reacting the azide 3.1 with an intermediate 3.2 in a copper catalyzed “click reaction”. The azide may be isolated or generated and used without isolation. There are multiple methods available for the synthesis of azides 3.1 such as substitution reactions where the starting material has a suitable leaving group or diazo transfer reactions where primary amines can be directly converted to azides. Sometimes these approaches require an appropriate metal catalyst.

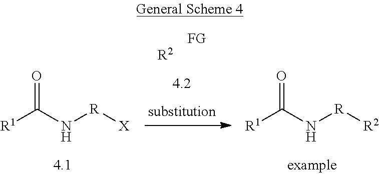

Certain compounds of Formula I were synthesized by substitution reaction with an appropriately functionalized intermediate 4.1 with appropriate starting materials 4.2. These reactions can be carried out using an appropriate base, solvent and temperature. Other conditions can employ an appropriate metal catalyst to effect the substitution.

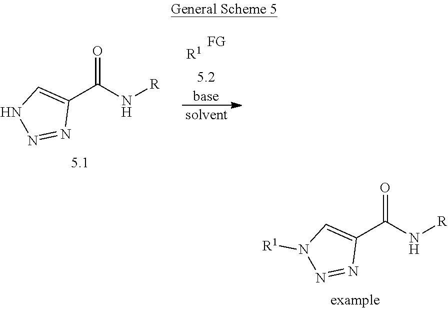

Certain compounds of Formula I were synthesized by functionalization of intermediates 5.1 with appropriately functionalized starting materials 5.2. These reactions can be carried out using an appropriate base, solvent and temperature. Other conditions can employ an appropriate metal catalyst to effect the functionalization.

### Intermediates

### Preparation of Intermediate (5-phenyl-1,3,4-thiadiazol-2-yl)methanamine hydrochloride (A.6

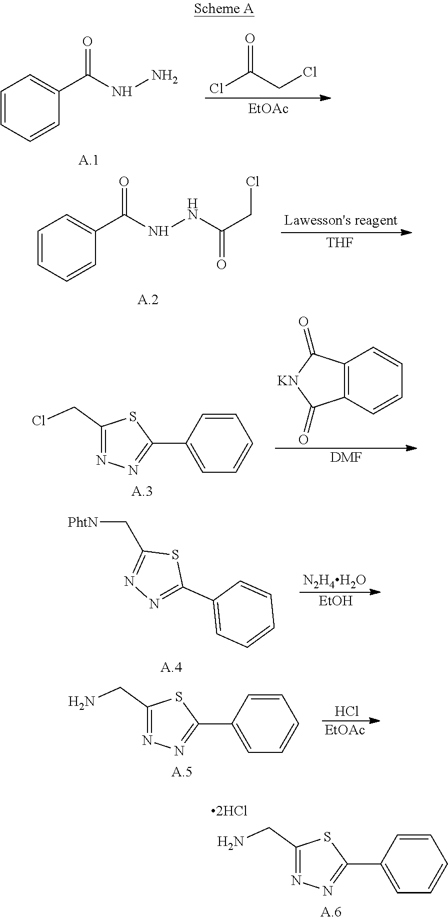

### Step 1. Preparation of N′-(2-chloroacetyl)benzohydrazide

A solution of benzohydrazide (600 g, 4.41 mol) in DCM (4.2 L) was cooled to 0° C. and 2-chloroacetyl chloride (597 g, 5.29 mol) was added dropwise at 0° C. The reaction mixture was stirred at 80° C. for 3 hours. The reaction was concentrated under reduced pressure and the crude product was triturated with MBTE (1.5 L). The solid was filtered, washed with MBTE (500 mL) and dried under vacuum to afford N′-(2-chloroacetyl)benzohydrazide. LC/MS (m/z): 213 (M+H)⁺

### Step 2. Preparation of 2-(chloromethyl)-5-phenyl-1,3,4-thiadiazole

Lawesson's reagent (1.04 kg, 2.57 mol) was added to a solution of N′-(2-chloroacetyl)benzohydrazide (780 g, 3.67 mol) in THF (7.8 L) at 20° C. and the reaction was stirred for 3 hours. The reaction was quenched by the addition saturated NaHCO₃ solution (7.80 L) and extracted with EtOAc (2×3 L). The combined organic fractions were washed with Brine (2×3 L), dried over anhydrous Na₂SO₄, filtered and concentrated. The residue was purified by silica gel chromatography (eluting ethyl acetate in petroleum ether) to afford 2-(chloromethyl)-5-phenyl-1,3,4-thiadiazole. LC/MS (m/z): 211 (M+H)⁺

### Step 3. Preparation of 2-((5-phenyl-1,3,4-thiadiazol-2-yl)methyl)isoindoline-1,3-dione

Potassium 1,3-dioxoisoindolin-2-ide (338 g, 1.82 mol) was added to a solution of 2-(chloromethyl)-5-phenyl-1,3,4-thiadiazole in DMF (3.2 L) at 0° C. The reaction was then heated to 60° C. for 3 hours. The reaction was cooled to 0° C., distilled water (3.2 L) was added, the mixture was warmed to 20° C. and stirred for 10 minutes. The solid was filtered, washed with distilled water (2 L) and dried under vacuum to afford crude 2-((5-phenyl-1,3,4-thiadiazol-2-yl)methyl)isoindoline-1,3-dione.

### Step 4. Preparation of (5-phenyl-1,3,4-thiadiazol-2-yl)methanamine

Two reactions were carried out in parallel. N₂H₄·H₂O (80.4 g, 1.57 mol, 98% purity) was added to a solution of 2-((5-phenyl-1,3,4-thiadiazol-2-yl)methyl)isoindoline-1,3-dione (232 g, 0.722 mol) in EtOH (3.48 L) at 20° C. The reaction was heated to 80° C. for 1 hour. The reactions were cooled to room temperature, combined and filtered. The filtrate was concentrated and the residue was triturated with EtOAc (4 L) and then filtered. The filtrate was concentrated to afford (5-phenyl-1,3,4-thiadiazol-2-yl)methanamine. LC/MS (m/z): 192 (M+H)⁺

### Step 5. Preparation of (5-phenyl-1,3,4-thiadiazol-2-yl)methanamine dihydrochloride

HCl (4 M in EtOAc, 1.25 L, 5 mol) was added to a solution of (5-phenyl-1,3,4-thiadiazol-2-yl)methanamine (240 g, 1.25 mol) in EtOAc (3.6 L) at 20° C. over 0.5 hours. The reaction was stirred for 1 hour. The mixture was filtered and the solid was triturated with MBTE (1.5 L) for 1 hour. The mixture was filtered and dried under vacuum to provide (5-phenyl-1,3,4-thiadiazol-2-yl)methanamine dihydrochloride. ¹H NMR (400 MHz, MeOD) δ 7.97-8.01 (m, 2H), 7.52-7.61 (m, 3H), 5.02 (s, 2H). LC/MS (m/z): 192 (M+H)⁺.

### Preparation of Intermediate N-((5-phenyl-1,3,4-thiadiazol-2-yl)methyl)propiolamide (B.1

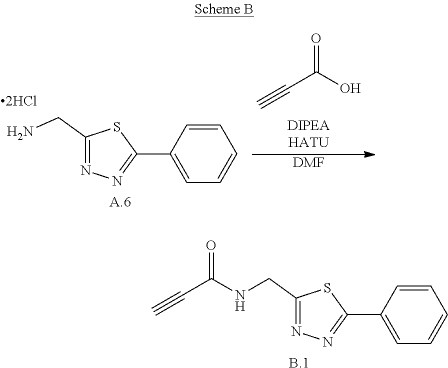

DIPEA (1020 g, 7.87 mol) and HATU (691 g, 1.82 mol) were added to a solution of (5-phenyl-1,3,4-thiadiazol-2-yl)methanamine hydrochloride (400 g, 1.51 mol) and propiolic acid (117 g, 1.67 mol) in DMF (2.8 L) at 25° C. and the reaction was stirred for 12 hours. The reaction was poured onto ice/water (600 mL) and extracted with DCM (3×200 mL). The combined organic fractions were washed with brine (3×200 mL), dried over sodium sulfate, filtered and concentrated. The residue was purified by silica gel chromatography (eluting ethyl acetate in petroleum ether) to afford N-((5-phenyl-1,3,4-thiadiazol-2-yl)methyl)propiolamide. ¹H NMR (499 MHz, DMSO) δ 9.75-9.66 (m, 1H), 8.01-7.93 (m, 2H), 7.60-7.51 (m, 3H), 4.73 (d, J=5.9 Hz, 2H), 4.31 (s, 1H). LC/MS (m/z): 244 (M+H)⁺

### Preparation of Intermediate (5-(pyridin-4-yl)-1,3,4-thiadiazol-2-yl)methanamine dihydrochloride (C.4

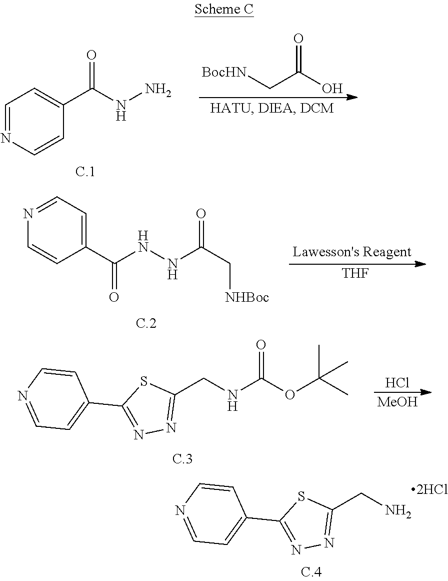

### Step 1. Preparation of tert-butyl (2-(2-isonicotinoylhydrazineyl)-2-oxoethyl carbamate

A 20 L 4-necked round-bottom flask was purged and maintained with an inert atmosphere of nitrogen. The flask was charged with (tert-butoxycarbonyl)glycine (1064.5 g, 6.076 mol), DCM (10 L), DIEA (2356.1 g, 18.23 mol), isonicotinohydrazide (1000 g, 7.292 mol) and HATU (3465.7 g, 9.115 mol). The reaction was stirred overnight at room temperature and the mixture was quenched with water (8 L) and extracted with DCM (3×5 L). The combined organic fractions were washed with brine (1×5 L), dried over anhydrous Na₂SO₄, filtered and concentrated. The residue was purified by silica gel chromatography (eluting MeOH in DCM) to afford tert-butyl (2-(2-isonicotinoylhydrazineyl)-2-oxoethyl)carbamate. LC/MS (m/z): 295 (M+H)⁺.

### Step 2. Preparation of tert-butyl ((5-(pyridin-4-yl)-1,3,4-thiadiazol-2-yl)methyl)carbamate

A 20 L 4-necked round-bottom flask was purged and maintained under an inert atmosphere of nitrogen. The flask was charged with tert-butyl ((5-(pyridin-4-yl)-1,3,4-thiadiazol-2-yl)methyl)carbamate (1.3 kg, 4.417 mol), THE (13 L) and Lawesson's Reagent (1.07 kg, 2.650 mol). The reaction was stirred at 70° C. for 16 hours. The reaction mixture was quenched with sat. NaHCO₃ (50 mL) and extracted with EtOAc (3×50 mL). The combined organic phases were washed with brine (1×50 mL), dried over anhydrous Na₂SO₄, filtered and concentrated. The residue was purified by silica gel chromatography (eluting EtOAc in petroleum ether) to afford tert-butyl ((5-(pyridin-4-yl)-1,3,4-thiadiazol-2-yl)methyl)carbamate. LC/MS (m/z): 293 (M+H)⁺.

### Step 3. Preparation of (5-(pyridin-4-yl)-1,3,4-thiadiazol-2-yl)methanamine dihydrochloride

A 10 L 4-necked round-bottom flask was purged and maintained under an inert atmosphere of nitrogen. The flask was charged with tert-butyl ((5-(pyridin-4-yl)-1,3,4-thiadiazol-2-yl)methyl)carbamate (600 g, 2.05 mol) and HCl (6 L, 24 mol, 4 M in MeOH) was added at room temperature and the mixture was stirred for 2 hours. The mixture was concentrated to afford (5-(pyridin-4-yl)-1,3,4-thiadiazol-2-yl)methanamine dihydrochloride. LC/MS (m/z): 193 (M+H)⁺. ¹H NMR (400 MHz, Deuterium Oxide) δ 8.92 (d, J=6.8 Hz, 2H), 8.54 (d, J=6.8 Hz, 2H), 4.82 (s, 2H).

Examples shown in Intermediate Table C below, were or may be prepared according to procedures analogous to those outlined in Scheme C above using the appropriate starting materials, described in the Preparations or Intermediates above, or as obtained from commercial sources.

Intermediate Table C

| | | | Mass |
|---|---|---|---|
| Example | Structure | Name | [M + H]⁺ |
| C.5 | 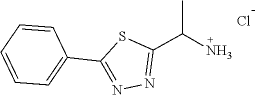 | 1-(5-phenyl-1,3,4-thiadiazol- 2-yl)ethan-1-aminium chloride | 206 |
| C.6 | 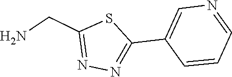 [SMILES: NCc1nnc(-c2cccnc2)s1] | (5-(pyridin-3-yl)-1,3,4- thiadiazol-2-yl)methanamine | Used directly |

### Preparation of Intermediate 1-(2,6-dimethylpyridin-3-yl)-1H-1,2,3-triazole-4-carboxylic acid hydrochloride (D.4

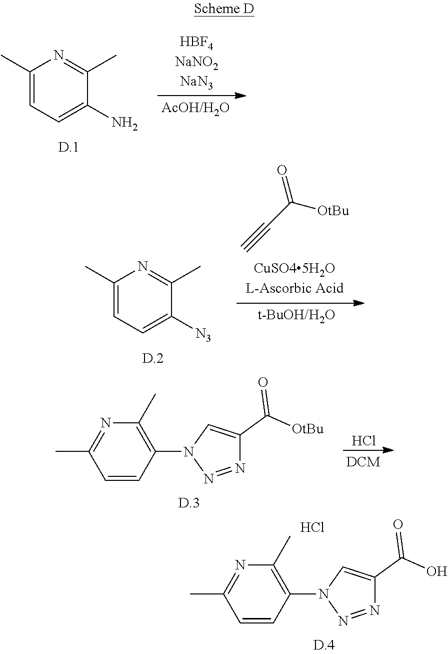

### Step 1. Preparation of 3-azido-2,6-dimethylpyridine

HBF₄ (299 g, 1.65 mol, 212 mL, 48% purity) was added dropwise to a solution of 2,6-dimethylpyridin-3-amine (40 g, 0.33 mol) in AcOH (180 mL) and distilled H₂O (120 mL) at 0° C. and the solution was stirred for 0.5 hours. A solution of NaNO₂ (24.8 g, 0.36 mol) in distilled H₂O (80.0 mL) was added dropwise to the at 0° C. and the reaction was stirred for 0.5 hours. A solution of NaN₃ (21.2 g, 0.33 mol) in distilled H₂O (80.0 mL) was added dropwise at 0° C. and the reaction was stirred for 0.5 hours. The pH was adjusted to 6 with 4 N NaOH solution. The mixture was filtered and the filtrate was used was used directly in the next step. LC/MS (m/z): 149 (M+H)⁺.

### Step 2. Preparation of tert-butyl 1-(2,6-dimethylpyridin-3-yl)-1H-1,2,3-triazole-4-carboxylate

T-BuOH (500 mL), distilled H₂O (1.5 L), tert-butyl propiolate (33 g, 261 mmol), CuSO₄·5H₂O (65.4 g, 261 mmol) and L-Ascorbic Acid (51.9 g, 261 mmol) was added to the above flask containing 3-azido-2,6-dimethylpyridine. The reaction was stirred at 50° C. for 2 hours. The reaction was quenched with NH₄OH solution (38% wt %) and the pH was adjusted to pH=8, CELITE was added and the mixture was stirred for 20 minutes. The suspension was filtered, the filter cake was washed with EtOAc (1 L) and the filtrate was concentrated. The residue was purified by silica gel chromatography (eluting EtOAc in petroleum ether) to afford tert-butyl 1-(2,6-dimethylpyridin-3-yl)-1H-1,2,3-triazole-4-carboxylate. LC/MS (m/z): 275 (M+H)⁺.

### Step 3. Preparation of 1-(2,6-dimethylpyridin-3-yl)-1H-1,2,3-triazole-4-carboxylic acid hydrochloride

HCl (1.22 L, 4.9 mol, 4 M in dioxane) was added to a solution of tert-butyl 1-(2,6-dimethylpyridin-3-yl)-1H-1,2,3-triazole-4-carboxylate (153 g, 558 mmol) in DCM (600 mL) and the reaction was stirred at 25° C. for 12 hours. The suspension was filtered, the filter cake was washed with DCM (200 mL), and the solid was dried at 45° C. under vacuum to afford 1-(2,6-dimethylpyridin-3-yl)-1H-1,2,3-triazole-4-carboxylic acid hydrochloride. LC/MS (m/z): 219 (M+H)⁺. ¹H NMR (400 MHz, DMSO) δ 9.15 (s, 1H), 8.10 (d, J=8.8 Hz, 1H), 7.56 (d, J=8.4 Hz, 1H), 2.65 (s, 3H), 2.44 (s, 3H).

Intermediates shown in Intermediate Table D below, were prepared according to procedures analogous to those outlined in Scheme D above using the appropriate starting materials, described in the Preparations or Intermediates above, or as obtained from commercial sources.

Intermediate Table D

| | | | Mass |
|---|---|---|---|
| Example | Structure | Name | [M + H]⁺ |
| D.5 | 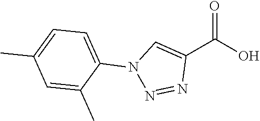 [SMILES: Cc1ccc(-n2cc(C(=O)O)nn2)c(C)c1] | N-((5-bromo-1,3,4- thiadiazol-2-yl)methyl)-1- methyl-1H-1,2,3-triazole-4- carboxamide | 218 |
| D.6 | 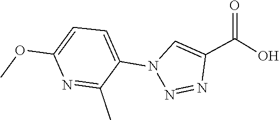 [SMILES: COc1ccc(-n2cc(C(=O)O)nn2)c(C)n1] | 1-(6-methoxy-2- methylpyridin-3-yl)-1H- 1,2,3-triazole-4-carboxylic acid | 235 |
| D.7 | 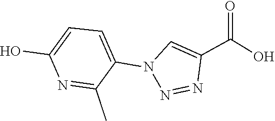 [SMILES: Cc1nc(O)ccc1-n1cc(C(=O)O)nn1] | 1-(6-hydroxy-2- methylpyridin-3-yl)-1H- 1,2,3-triazole-4-carboxylic acid | 221 |
| D.8 | 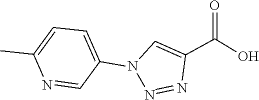 | 1-(6-methylpyridin-3-yl)-1H- 1,2,3-triazole-4-carboxylic acid | 205 |
| D.9 | 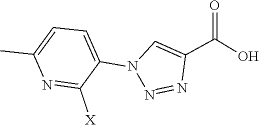 | 1-(2-bromo-6-methylpyridin- 3-yl)-1H-1,2,3-triazole-4- carboxylic acid and 1-(2- chloro-6-methylpyridin-3- yl)-1H-1,2,3-triazole-4- carboxylic acid | 283, 239 |
| | X = Br, Cl | | |
| | mixture | | |
| D.10 | 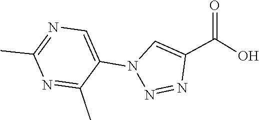 [SMILES: Cc1ncc(-n2cc(C(=O)O)nn2)c(C)n1] | 1-(2,4-dimethylpyrimidin-5- yl)-1H-1,2,3-triazole-4- carboxylic acid | 220 |
| D.11 | 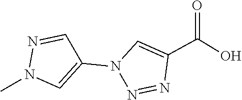 | 1-(1-methyl-1H-pyrazol-4- yl)-1H-1,2,3-triazole-4- carboxylic acid | 194 |

### Preparation of Intermediate 1-(2,2-difluoroethyl)-1H-1,2,3-triazole-4-carboxylic acid (E.2

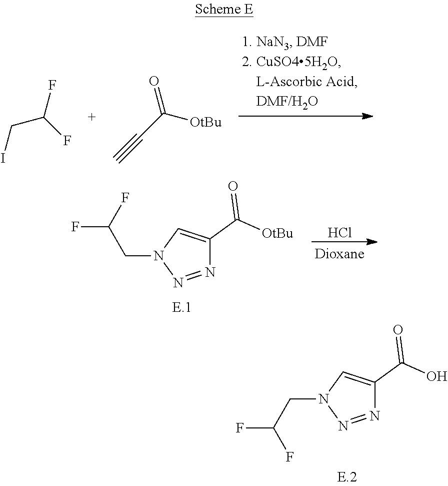

### Step 1. Preparation of tert-butyl 1-(2,2-difluoroethyl)-1H-1,2,3-triazole-4-carboxylate

A round bottomed flask with stir bar and rubber septum was charged with 1,1-difluoro-2-iodoethane (9.51 g, 49.5 mmol), DMF (19.82 ml) and sodium azide. The vessel was sealed and heated to 60° C. overnight. The reaction was cooled to room temperature tert-butyl propiolate (2.72 mL, 19.82 mmol) was added, followed by DMF (19.8 mL) and aqueous solutions of copper(II) sulfate (4.95 mL, 1.98 mmol, 0.4 M) and L-sodium ascorbate (4.95 ml, 3.96 mmol, 0.8 M). The reaction was stirred at room temperature overnight. Distilled H₂O (50 mL) was added and the precipitate was collected by vacuum filtration. The filter cake was washed with a 15 wt % aqueous NH₄OH solution (3×50 mL), then distilled H₂O (1×50 mL), and the solid was dried on the frit under vacuum with a stream of nitrogen blowing over top) to afford tert-butyl 1-(2,2-difluoroethyl)-1H-1,2,3-triazole-4-carboxylate. LC/MS (m/z): 256 (M+Na)+.

### Step 2. Preparation of 1-(2,2-difluoroethyl)-1H-1,2,3-triazole-4-carboxylic acid

HCl (15 mL, 60.0 mmol, 4 M in dioxane) was added to a solution of tert-butyl 1-(2,2-difluoroethyl)-1H-1,2,3-triazole-4-carboxylate (4.68 g, 20.1 mmol) in 1,4-dioxane (30 mL) and the reaction was heated to 50° C. overnight. The reaction was cooled room temperature, concentrated under reduced pressure and triturated with diethyl ether (50 mL), the solid was collect by filtration and washed with additional diethyl ether (3×50 mL) to afford 1-(2,2-difluoroethyl)-1H-1,2,3-triazole-4-carboxylic acid. LC/MS (m/z): 178 (M+H)⁺. ¹H NMR (499 MHz, DMSO) δ 8.70 (s, 1H), 6.69-6.29 (m, 1H), 5.11-4.89 (m, 2H).

Intermediates shown in Intermediate Table E below, were prepared according to procedures analogous to those outlined in Scheme E above using the appropriate starting materials, described in the Preparations or Intermediates above, or as obtained from commercial sources.

Intermediate Table E

| | | | Mass |
|---|---|---|---|
| Example | Structure | Name | [M + H]⁺ |
| E.3 | 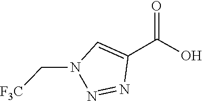 [SMILES: O=C(O)c1cn(CC(F)(F)F)nn1] | N-((5-bromo-1,3,4- thiadiazol-2-yl)methyl)-1- methyl-1H-1,2,3-triazole-4- carboxamide | 303 |
| E.4 | 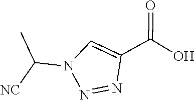 | 1-(1-cyanoethyl)-1H-1,2,3- triazole-4-carboxylic acid | 167 |
| E.5 | 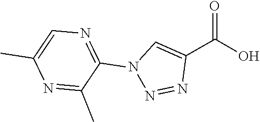 [SMILES: Cc1cnc(-n2cc(C(=O)O)nn2)c(C)n1] | 1-(3,5-dimethylpyrazin-2- yl)-1H-1,2,3-triazole-4- carboxylic acid | 220 |

### Preparation of Intermediate N-((5-bromo-1,3,4-thiadiazol-2-yl)methyl)-1-(2,6-dimethylpyridin-3-yl)-1H-1,2,3-triazole-4-carboxamide (F.2

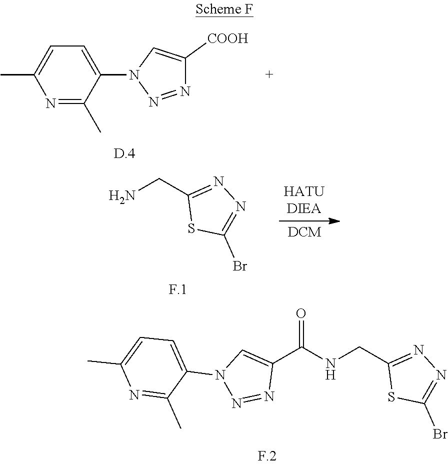 [SMILES: Cc1ccc(-n2cc(C(=O)O)nn2)c(C)n1 ; NCc1nnc(Br)s1 ; Cc1ccc(-n2cc(C(=O)NCc3nnc(Br)s3)nn2)c(C)n1]

A mixture of 1-(2,6-dimethylpyridin-3-yl)-1H-1,2,3-triazole-4-carboxylic acid (1.5 g, 6.87 mmol), HATU (3.92 g, 10.31 mmol), DIEA (3.60 mL, 20.62 mmol) and (5-bromo-1,3,4-thiadiazol-2-yl)methanamine (1.33 g, 6.87 mmol) in DCM (10 mL) was stirred at 25° C. for 2 h to give a mixture. The mixture was filtered and the filtrate was concentrated under reduced pressure. The residue was purified by silica gel chromatography (eluenting ethyl acetate in petroleum ether) to afford N-((5-bromo-1,3,4-thiadiazol-2-yl)methyl)-1-(2,6-dimethylpyridin-3-yl)-1H-1,2,3-triazole-4-carboxamide. LC/MS (m/z): 394 (M+H)⁺. ¹H NMR (400 MHz, MeOD) δ 8.76 (s, 1H), 7.80 (d, J=8.23 Hz, 1H), 7.37 (d, J=8.23 Hz, 1H), 4.97-5.00 (m, 2H), 2.62 (s, 3H), 2.40 (s, 3H)

Examples shown in Intermediate Table F below, were or may be prepared according to procedures analogous to those outlined in Scheme F above using the appropriate starting materials, described in the Preparations or Intermediates above, or as obtained from commercial sources.

Intermediate Table F

| | | | Mass |
|---|---|---|---|
| Example | Structure | Name | [M + H]⁺ |
| F.3 | 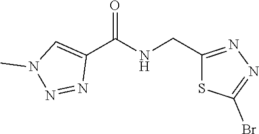 [SMILES: Cn1cc(C(=O)NCc2nnc(Br)s2)nn1] | N-((5-bromo-1,3,4- thiadiazol-2-yl)methyl)-1- methyl-1H-1,2,3-triazole-4- carboxamide | 303 |

### Preparation of Intermediate N-((6-chloropyridazin-3-yl)methyl)-1-(2,6-dimethylpyridin-3-yl)-1H-1,2,3-triazole-4-carboxamide (G.4

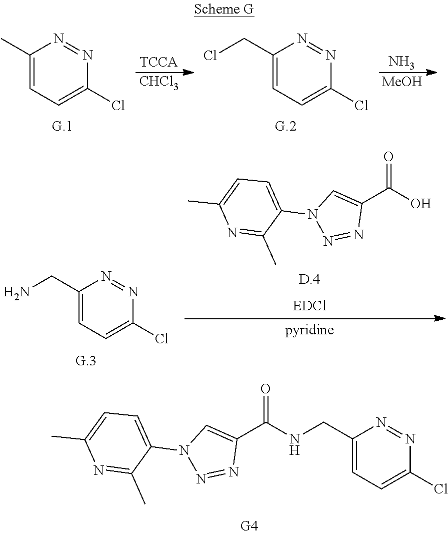

### Step 1. Preparation of 3-chloro-6-(chloromethyl)pyridazine

A mixture of 3-chloro-6-methylpyridazine (20 g, 156 mmol) and TCCA (14.46 g, 62.2 mmol) in CHCl₃ (250 mL) was stirred at 60° C. for 16 hours. The mixture was filtered and the filtrate was concentrated under reduced pressure. The residue was purified by silica gel chromatography (eluting EtOAc in petroleum ether) to afford give 3-chloro-6-(chloromethyl)pyridazine. LC/MS (m/z): 163 (M+H)⁺.

### Step 2. Preparation of (6-chloropyridazin-3-yl)methanamine

A mixture of 3-chloro-6-(chloromethyl)pyridazine (11.8 g, 72.4 mmol) in ammonia in MeOH (300 mL, 72.4 mmol, 7M) was stirred at 25° C. for 16 hours. The solvent was removed under reduced pressure and the residue was dissolved in water (150 mL) and EtOAc (50 mL). The organic layer was separated and the aqueous phase was extracted with EtOAc (3×50 mL) and 20:1 DCM/MeOH (2×50 mL), the aqueous phase was lyophilized to afford (6-chloropyridazin-3-yl)methanamine which was used in next step without further purification. LC/MS (m/z): 144 (M+H)⁺.

### Step 3. Preparation of N-((6-chloropyridazin-3-yl)methyl)-1-(2,6-dimethylpyridin-3-yl)-1H-1,2,3-triazole-4-carboxamide

EDCI (6.33 g, 33.0 mmol) and pyridine (5.00 mL, 61.9 mmol) were added to a solution of 1-(2,6-dimethylpyridin-3-yl)-1H-1,2,3-triazole-4-carboxylic acid (4.5 g, 20.62 mmol) in DMF (50 mL) at 25° C. over 1 minutes. After stirring for 2 minutes at 25° C., (6-chloropyridazin-3-yl)methanamine (2.96 g, 20.62 mmol) was added to the mixture at 25° C. and the reaction was stirred for 3 hours. The solvent was removed under reduced pressure and the residue was dissolved in 1:1 DCM/MeOH (100 mL) and water (100 mL). The organic layer was separated and the aqueous layer was extracted with 1:1 DCM/MeOH (5×60 mL) and the combined organic layers were washed with brine (100 mL), dried over anhydrous Na₂SO₄, filtered and concentrated under reduced pressure to afford N-((6-chloropyridazin-3-yl)methyl)-1-(2,6-dimethylpyridin-3-yl)-1H-1,2,3-triazole-4-carboxamide. LC/MS (m/z): 344 (M+H)⁺.

### Preparation of Intermediate 5-(2,6-dimethylpyridin-3-yl)isoxazole-3-carboxylic acid (H.4

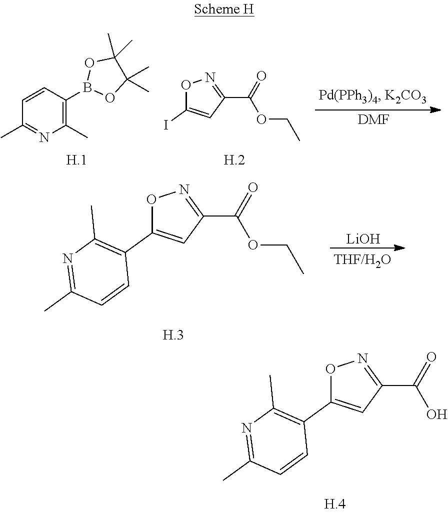

### Step 1. Preparation of ethyl 5-(2,6-dimethylpyridin-3-yl)isoxazole-3-carboxylate

5-iodo-isoxazole-3-carboxylic acid ethyl ester (120 mg, 0.448 mmol), tetrakis(triphenylphosphine)palladium(0) (104 mg, 0.090 mmol) and K₂CO₃ (186 mg, 1.344 mmol) were added to a solution of 2,6-dimethyl-3-(4,4,5,5-tetramethyl-1,3,2-dioxaborolan-2-yl)pyridine (125 mg, 0.536 mmol) in DMF (1.1 mL). The resulting mixture was stirred at 80° C. for 12 hours. The reaction mixture was diluted with ethyl acetate (5 mL). The layers were separated. The aqueous layer was extracted with ethyl acetate (3×5 mL). The combined organic layers were washed with brine, dried over Na₂SO₄, filtered and concentrated under reduced pressure to afford ethyl 5-(2,6-dimethylpyridin-3-yl)isoxazole-3-carboxylate as an oil. LC/MS (m/z): 247 (M+H)⁺.

### Step 2. Preparation of 5-(2,6-dimethylpyridin-3-yl)isoxazole-3-carboxylic acid

Lithium hydroxide monohydrate (11.63 mg, 0.277 mmol) was added to a vial containing ethyl 5-(2,6-dimethylpyridin-3-yl)isoxazole-3-carboxylate (27.3 mg, 0.111 mmol). The mixture was suspended in a mixture of THF (277 μL) and water (277 μL). The reaction mixture was allowed to stir and slowly dissolve overnight at 22° C. The reaction mixture was adjusted to a pH ˜4-5 with HCl. The mixture was concentrated under reduced pressure to afford 5-(2,6-dimethylpyridin-3-yl)isoxazole-3-carboxylic acid.

### Preparation of Intermediate 1-(1-methoxy-2-methyl-1-oxopropan-2-yl)-1H-1,2,3-triazole-4-carboxylic acid (I.3

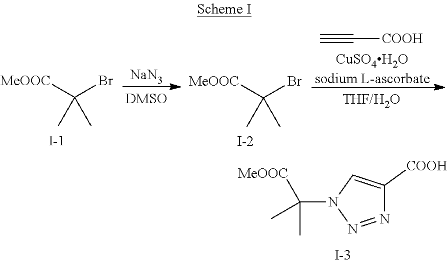

### Step 1. Preparation of methyl 2-azido-2-methylpropanoate

A solution of methyl 2-bromo-2-methylpropanoate (2 g, 11 mmol) and NaN₃ (1.590 g, 24.46 mmol) in DMSO (20 mL) was stirred at 50° C. for 2 hours. The reaction mixture was quenched with sat. Na₂CO_{3(aq)} (50 mL), extracted with EtOAc (2×40 mL) and the organic phase was dried over Na₂SO₄ and concentrated to afford methyl 2-azido-2-methylpropanoate.

### Step 2. Preparation of 1-(1-methoxy-2-methyl-1-oxopropan-2-yl)-1H-1,2,3-triazole-4-carboxylic acid

A mixture of but-3-ynoic acid (200 mg, 2.379 mmol), methyl 2-azido-2-methylpropanoate (511 mg, 3.57 mmol), copper(II) sulfate pentahydrate (38.0 mg, 0.238 mmol) and (+)-sodium 1-ascorbate (47.1 mg, 0.238 mmol) in water (1 mL) and t-BuOH (9 mL) was stirred for 16 hours at 50° C. The mixture was filtered and the filtrate was purified via reverse phase HPLC (eluting acetonitrile in water with 0.1% TFA to afford 1-(1-methoxy-2-methyl-1-oxopropan-2-yl)-1H-1,2,3-triazole-4-carboxylic acid. LCMS (ESI) m/z: 214 (M+H)⁺.

### Preparation of Intermediate N-((5-phenyl-1,3,4-thiadiazol-2-yl)methyl)-1H-1,2,3-triazole-4-carboxamide (J.2

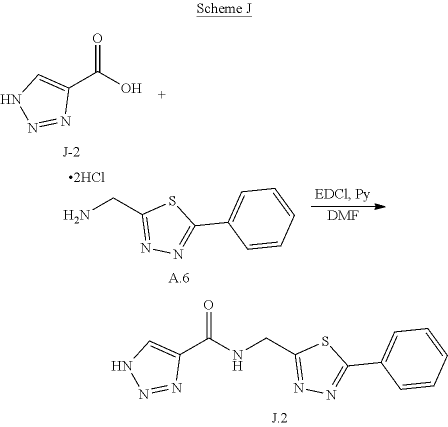

(5-phenyl-1,3,4-thiadiazol-2-yl)methanamine dihydrochloride (1.31 g, 4.99 mmol) was added to a mixture of 1H-1,2,3-triazole-4-carboxylic acid (0.564 g, 4.99 mmol), pyridine (1.614 mL, 19.96 mmol) and EDC (1.435 g, 7.48 mmol) in DMF (15 mL) at 20° C. The resulting mixture was stirred at 20° C. for 2 hours. The reaction mixture was filtered and the residue was purified via reverse phase HPLC (eluting acetonitrile in water with 0.1% TFA) to afford N-((5-phenyl-1,3,4-thiadiazol-2-yl)methyl)-1H-1,2,3-triazole-4-carboxamide. LCMS (ESI) m/z: 287 (M+H)⁺.

### Preparation of Intermediate 1-(2,6-dimethylpyridin-3-yl)-N-(prop-2-yn-1-yl)-1H-1,2,3-triazole-4-carboxamide (K.2

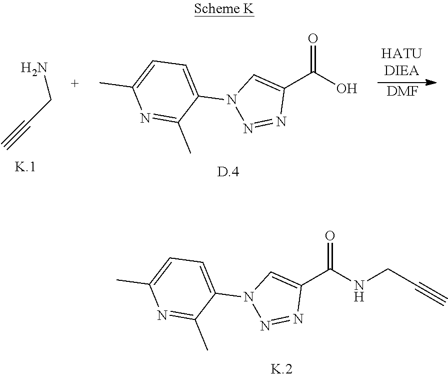

DIEA (0.096 mL, 0.550 mmol) and HATU (78 mg, 0.206 mmol) was added to a solution of 1-(2,6-dimethylpyridin-3-yl)-1H-1,2,3-triazole-4-carboxylic acid (30 mg, 0.137 mmol) in DMF (1 mL) at 25° C. over 2 minutes. After stirring for 5 min at 25° C., prop-2-yn-1-amine (9.69 μL, 0.151 mmol) was added to the mixture at 25° C. The resulting mixture was stirred for another 2 hours. The mixture was filtered and the filtrate was purified via reverse phase HPLC (eluting acetonitrile in water with 0.1% TFA) to afford 1-(2,6-dimethylpyridin-3-yl)-N-(prop-2-yn-1-yl)-1H-1,2,3-triazole-4-carboxamide. LCMS (ESI) m/z: 256 (M+H)⁺.

### Preparation of Intermediate 5-methylisothiazole-3-carboxylic acid (L.4

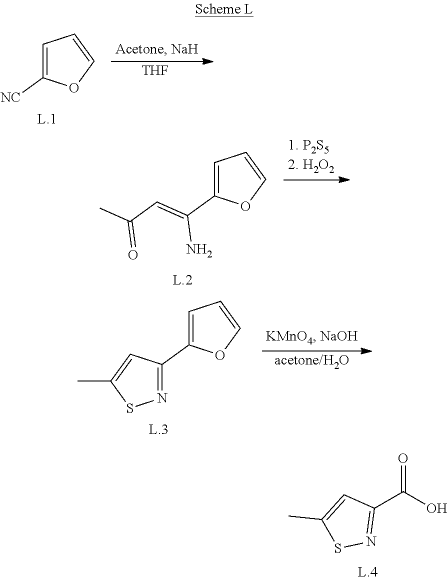

### Step 1. Preparation of (Z)-4-amino-4-(furan-2-yl)but-3-en-2-one

NaH (0.516 g, 12.89 mmol) was added to a mixture of furan-2-carbonitrile (1 g, 11 mmol) and propan-2-one (0.949 mL, 12.89 mmol) in THF (20 mL) at 25° C. The resulting mixture was stirred for another 16 h at 65° C. The reaction mixture was quenched with H₂O (50 mL) and extracted with EtOAc (3×50 mL). The combined organic phases were washed with brine (20 mL), dried over anhydrous Na₂SO₄, filtered and concentrated under reduced pressure. The residue was purified by flash silica gel chromatography (eluting ethyl acetate in petroleum ether) to afford (Z)-4-amino-4-(furan-2-yl)but-3-en-2-one LCMS (ESI) m/z: 152 (M+H)⁺.

### Step 2. Preparation of 3-(furan-2-yl)-5-methylisothiazole

A mixture of (Z)-4-amino-4-(furan-2-yl)but-3-en-2-one (300 mg, 1.985 mmol) and P₂S₅ (221 mg, 0.992 mmol) in THF (10 mL) was stirred at 25° C. for 12 hours. The mixture was concentrated under reduced pressure to afford crude (Z)-4-amino-4-(furan-2-yl)but-3-ene-2-thione which was used in the next step without further purification.

### Step 3. Preparation of 3-(furan-2-yl)-5-methylisothiazole

H₂O₂ (4.79 mL, 46.9 mmol) was added to a mixture of (Z)-4-amino-4-(furan-2-yl)but-3-ene-2-thione (300 mg, 1.794 mmol) in MTBE (5 mL) at 25° C. The resulting mixture was stirred at 25° C. for 16 hours. The reaction mixture was quenched with saturated Na₂SO₃ (20 mL) and extracted with EtOAc (3×20 mL). The combined organic phases were washed with brine (10 mL), dried over anhydrous Na₂SO₄, filtered and concentrated under reduced pressure. The residue was purified by silica gel chromatography (eluting ethyl acetate in petroleum ether) to give afford 3-(furan-2-yl)-5-methylisothiazole. LCMS (ESI) m/z: 166 (M+H)⁺.

### Step 4. Preparation of 5-methylisothiazole-3-carboxylic acid

KMnO₄ (287 mg, 1.816 mmol) was added to a mixture of 3-(furan-2-yl)-5-methylisothiazole (150 mg, 0.908 mmol) in acetone (1 mL) and water (2 mL) at 25° C. The resulting mixture was stirred at 25° C. for 1.5 hours. Then NaOH (2 mL, 2 M) was added and heated to 50° C. for 48 hours. The resulting mixture was acidified with 2M HCl and extracted with ethyl acetate (3×20 mL). The residue was purified via reverse phase HPLC (eluting acetonitrile in water with 0.1% TFA modifier) to afford 5-methylisothiazole-3-carboxylic acid.

### Preparation of intermediate (5-(pyridin-4-yl)isoxazol-3-yl)methanamine (M.5

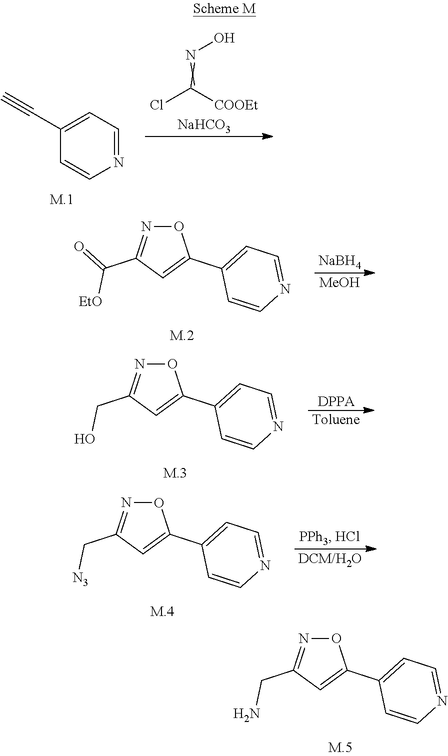

### Step 1. Preparation of ethyl 5-(pyridin-4-yl)isoxazole-3-carboxylate

A mixture of ethyl (E)-2-chloro-2-(hydroxyimino)acetate (4.89 g, 32.2 mmol) and 4-ethynylpyridine hydrochloride (1.5 g, 10.75 mmol), NaHCO₃ (2.71 g, 32.2 mmol) in EtOAc (20 mL) was stirred at 100° C. for 1 hour. The mixture was filtered and the filtrate was purified by flash silica gel chromatography (eluting ethyl acetate in petroleum ether) to afford ethyl 5-(pyridin-4-yl)isoxazole-3-carboxylate. ¹H NMR (400 MHz, CDCl₃) δ 8.80 (br d, J=5.25 Hz, 2H), 7.72 (d, J=5.96 Hz, 2H), 7.15 (s, 1H), 4.50 (q, J=7.15 Hz, 2H), 1.46 (t, J=7.15 Hz, 3H).

### Step 2. Preparation of (5-(pyridin-4-yl)isoxazol-3-yl)methanol

NaBH₄ (69.3 mg, 1.83 mmol) was added to a solution of ethyl 5-(pyridin-4-yl)isoxazole-3-carboxylate (100 mg, 0.458 mmol) in MeOH (10 mL) at 0° C. over 5 minutes. The reaction was warmed to 25° C. and stirred for 20 hours. The reaction mixture was quenched with HCl (2 mL, 1N). The solvent was removed under vacuum and the residue was purified via reverse phase HPLC (eluting acetonitrile in water with 0.1% TFA modifier) to afford (5-(pyridin-4-yl)isoxazol-3-yl)methanol. LCMS (ESI) m/z: 177 (M+H)⁺.

### Step 3. Preparation of 3-(azidomethyl)-5-(pyridin-4-yl)isoxazole

A mixture of (5-(pyridin-4-yl)isoxazol-3-yl)methanol (30 mg, 0.170 mmol), DBU (31.1 mg, 0.204 mmol) and DPPA (0.044 mL, 0.204 mmol) in toluene (2 mL) was stirred at 25° C. for 16 hours. The solvent was removed under vacuum and the residue was purified via reverse phase HPLC (eluting acetonitrile in water with 0.1% TFA modifier) to afford 3-(azidomethyl)-5-(pyridin-4-yl)isoxazole. LCMS (ESI) m/z: 202 (M+H)⁺.

### Step 4: Preparation of (5-(pyridin-4-yl)isoxazol-3-yl)methanamine

A mixture of 3-(azidomethyl)-5-(pyridin-4-yl)isoxazole (30 mg, 0.149 mmol), PPh₃ (19.56 mg, 0.075 mmol) and HCl (0.149 mL, 0.149 mmol) in DCM (3 mL) was stirred at 25° C. for 16 hours. The reaction mixture was quenched with water (20 mL) and extracted with DCM (3×15 mL). The combined aqueous phases were lyophilized to afford (5-(pyridin-4-yl)isoxazol-3-yl)methanamine that was used without further purification. LCMS (ESI) m/z: 176 (M+H)⁺.

### Preparation of Intermediate 1-(1-amino-2-methyl-1-oxopropan-2-yl)-1H-1,2,3-triazole-4-carboxylic acid (N3)

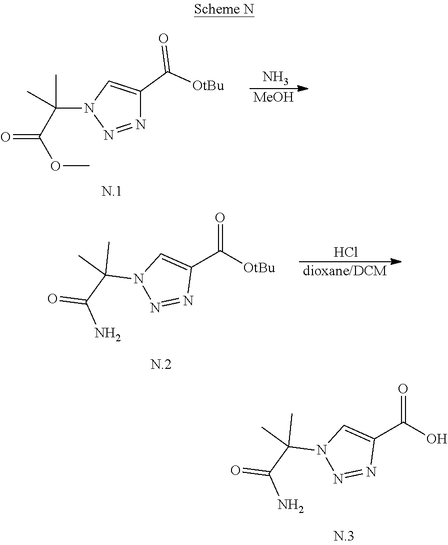

### Step 1. Preparation of tert-butyl 1-(1-amino-2-methyl-1-oxopropan-2-yl)-1H-1,2,3-triazole-4-carboxylate

A mixture of tert-butyl 1-(1-methoxy-2-methyl-1-oxopropan-2-yl)-1H-1,2,3-triazole-4-carboxylate (400 mg, 1.485 mmol) in NH₃ (3 mL, 21 mmol, 7 M in MeOH) was stirred at 25° C. for 12 hours. The mixture was concentrated under reduced pressure to afford tert-butyl 1-(1-amino-2-methyl-1-oxopropan-2-yl)-1H-1,2,3-triazole-4-carboxylate that was used without further purification. LCMS (ESI) m/z: 255 (M+H)⁺.

### Step 2. Preparation of 1-(1-amino-2-methyl-1-oxopropan-2-yl)-1H-1,2,3-triazole-4-carboxylic acid

A mixture of tert-butyl 1-(1-amino-2-methyl-1-oxopropan-2-yl)-1H-1,2,3-triazole-4-carboxylate (350 mg, 1.376 mmol) and HCl/dioxane (3 mL, 12 mmol, 4M in dioxane) in DCM (3 mL) was stirred at 20° C. for 12 hours. The mixture was concentrated under reduced pressure to afford 1-(1-amino-2-methyl-1-oxopropan-2-yl)-1H-1,2,3-triazole-4-carboxylic acid. LCMS (ESI) m/z: 199 (M+H)⁺.

### Preparation of Intermediate methyl 1-(difluoromethyl)-1H-1,2,3-triazole-4-carboxylate (O.3

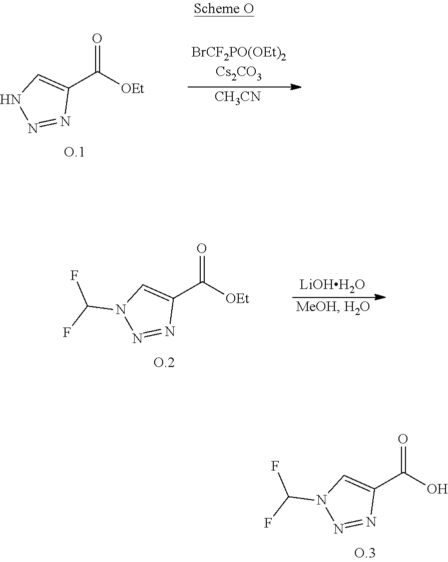

### Step 1. Preparation of methyl 1-(difluoromethyl)-1H-1,2,3-triazole-4-carboxylate

A mixture of methyl 1H-1,2,3-triazole-4-carboxylate (1.2 g, 9.44 mmol), Cs₂CO₃ (3.08 g, 9.44 mmol) and diethyl (bromodifluoromethyl)phosphonate (3.28 g, 12.27 mmol) in DMF (5 mL) was stirred at 20° C. for 12 hours. The reaction mixture was filtered, the filtrate was concentrated and the residue was purified via reverse phase HPLC (eluting acetonitrile in water with 0.1% TFA modifier) to afford methyl 1-(difluoromethyl)-1H-1,2,3-triazole-4-carboxylate. LCMS (ESI) m/z: 178 (M+H)⁺.

### Step 2. Preparation of methyl 1-(difluoromethyl)-1H-1,2,3-triazole-4-carboxylate

A mixture of methyl 1-(difluoromethyl)-1H-1,2,3-triazole-4-carboxylate (623 mg, 3.52 mmol) and LiOH·H₂O (148 mg, 3.52 mmol) in MeOH (6 mL) and water (2 mL) was stirred at 25° C. for 2 hours. The mixture was filtered and the filtrate was concentrated under reduced pressure to afford 1-(difluoromethyl)-1H-1,2,3-triazole-4-carboxylic acid which was used without further purification.

### Preparation of Intermediate 1-(2,6-dimethylpyridin-3-yl)-N-((5-(thiazol-2-yl)isoxazol-3-yl)methyl)-1H-1,2,3-triazole-4-carboxamide (P.3

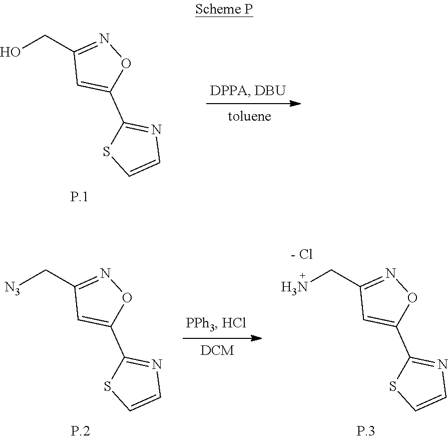 [SMILES: OCc1cc(-c2nccs2)on1 ; [N-]=[N+]=NCc1cc(-c2nccs2)on1 ; NCc1cc(-c2nccs2)on1]

### Step 1. Preparation of 3-(azidomethyl)-5-(thiazol-2-yl)isoxazole

A mixture of (5-(thiazol-2-yl)isoxazol-3-yl)methanol (114 mg, 0.626 mmol) and DBU (0.118 mL, 0.782 mmol), DPPA (0.169 mL, 0.782 mmol) in toluene (5 mL) was stirred at 25° C. for 16 hours. The reaction mixture was quenched with H₂O (5 mL) and extracted with EtOAc (3×5 mL). The combined organic phases were washed with brine (3 mL), dried over anhydrous Na₂SO₄, filtered and concentrated under reduced pressure to afford 3-(azidomethyl)-5-(thiazol-2-yl)isoxazole that was used without further purification. LCMS (ESI) m/z: 208 (M+H)⁺.

### Step 2. Preparation of (5-(thiazol-2-yl)isoxazol-3-yl)methanamine

Ph₃P (247 mg, 0.941 mmol) was added to a mixture of 3-(azidomethyl)-5-(thiazol-2-yl)isoxazole (130 mg, 0.627 mmol) in DCM (3 mL) and HCl (3 mL, 12 mmol, 4N) at 0° C. The resulting mixture was stirred at 25° C. for 16 hours. Then water (10 mL) was added and the mixture was extracted with DCM (3×10 mL). The combined the aqueous phases were lyophilized to afford (5-(thiazol-2-yl)isoxazol-3-yl)methanamine that was used without further purification. LCMS (ESI) m/z: 182 (M+H)⁺.

### Preparation of Intermediate 1-(trifluoromethyl)-1H-1,2,3-triazole-4-carboxylic acid (Q.2

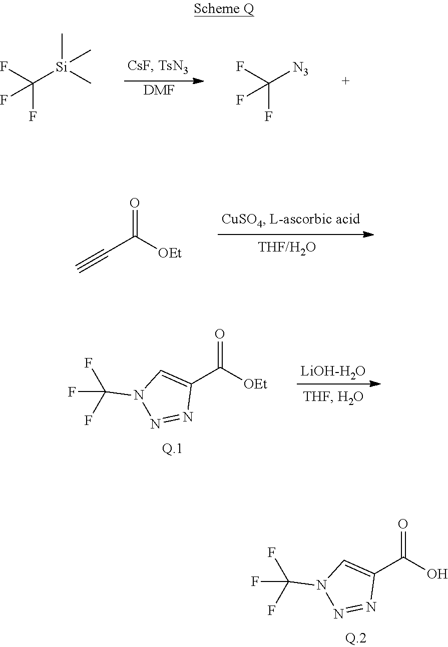

### Step 1. Preparation of azidotrifluoromethane

Cesium fluoride (534 mg, 3.52 mmol) was dried overnight at 120° C. under high vacuum in a two-neck round bottomed flask. The flask was cooled to room temperature, backfilled with argon, and charged with dry DMF (3 mL). The mixture was cooled to −60° C. and a cold solution of (trifluoromethyl)trimethylsilane (500 mg, 3.52 mmol) and TsN₃ (3.07 mL, 20 mmol) in dry DMF (6 mL) was added dropwise over 20 minutes. The reaction mixture was stirred at −60° C. to −30° C. for 4 hours. This solution was used directly in the next step.

### Step 2. Preparation ethyl 1-(trifluoromethyl)-1H-1,2,3-triazole-4-carboxylate

Copper(II) sulfate (0.259 g, 1.621 mmol) and a solution of (+)-sodium 1-ascorbate (0.321 g, 1.621 mmol) in water (1 mL) was added to a mixture of ethyl propiolate (1.590 g, 16.21 mmol) and azidotrifluoromethane (1.8 g, 16.2 mmol) in THF (9 mL) at 25° C. over 1 minute. The reaction was stirred for 4 hours at 20° C. The mixture was filtered, concentrated and the residue was purified via reverse phase HPLC (eluting acetonitrile in water with 0.1% TFA modifier) to afford ethyl 1-(trifluoromethyl)-1H-1,2,3-triazole-4-carboxylate. LCMS (ESI) m/z: 210 (M+H)⁺.

### Step 3. Preparation of 1-(trifluoromethyl)-1H-1,2,3-triazole-4-carboxylic acid

A mixture of ethyl 1-(trifluoromethyl)-1H-1,2,3-triazole-4-carboxylate (0.70 g, 3.35 mmol) and LiOH·H₂O (0.421 g, 10.04 mmol) in THF (2 mL) and water (2 mL) was stirred at 25° C. for 16 hours. The reaction mixture was quenched with 1 M HCl to pH ˜4 and extracted with EtOAc (5×3 mL). The combined organic phases were washed with brine (15 mL), dried over anhydrous Na₂SO₄, filtered and concentrated under reduced pressure. The residue was purified via reverse phase HPLC (eluting acetonitrile in water with 0.04% HCl modifier) to afford 1-(trifluoromethyl)-1H-1,2,3-triazole-4-carboxylic acid. LCMS (ESI) m/z: 182.0 (M+H)⁺.

### Preparation of Intermediate 1-(2,6-dimethylpyridin-3-yl)-1H-1,2,3-triazole-4-carboxylic acid hydrochloride (R.4

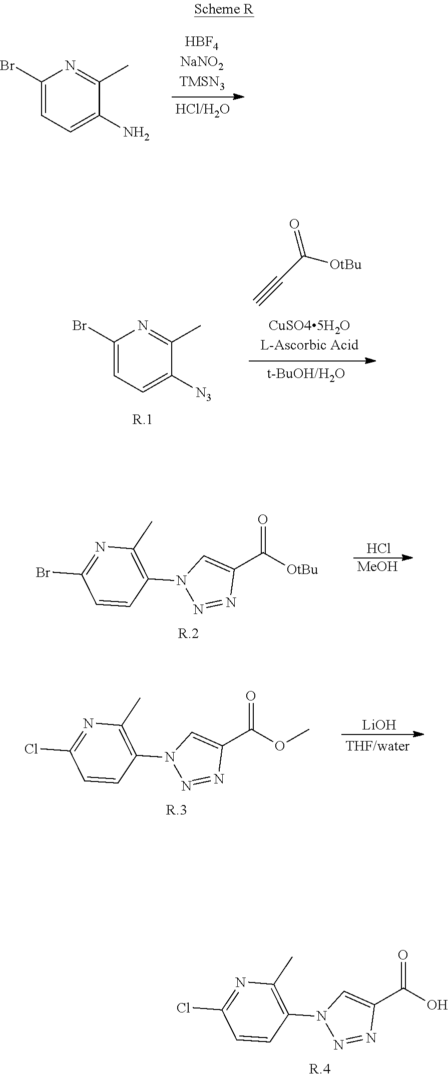

### Step 1. Preparation of 3-azido-6-bromo-2-methylpyridine

Sodium nitrite (1.383 g, 20.05 mmol) (dissolved in 20 mL water) was added to a solution of 6-bromo-2-methylpyridin-3-amine (2.5 g, 13.37 mmol) in HCl (6M) (35 mL) at 0° C. over 10 min. After stirring for 0.5 hours at 0° C., TMS-N3 (7.10 mL, 53.5 mmol) was added to the mixture at 0° C. The resulting mixture was stirred for another 8 hours. The mixture was basified with aqueous NaOH (pH >9) and extracted with EtOAc (3×50 mL). The combined organic phases were washed with brine (20 mL), dried over anhydrous Na₂SO₄, filtered and concentrated under reduced pressure. The residue was purified by flash silica gel chromatography (eluting EtOAc in petroleum ether) to afford 3-azido-6-bromo-2-methylpyridine LCMS (m/z): 213 (M+H)⁺.

### Step 2. Preparation of tert-butyl 1-(6-bromo-2-methylpyridin-3-yl)-1H-1,2,3-triazole-4-carboxylate

tert-butyl propiolate (1.658 g, 13.14 mmol) was added to a solution of 3-azido-6-bromo-2-methylpyridine (2.8 g, 13.14 mmol), (+)-sodium L-ascorbate (0.260 g, 1.314 mmol), copper(II) sulfate (0.210 g, 1.314 mmol) and in tert-BuOH (30 mL) and water (3 mL). After stirring for 16 hours at 50° C. the reaction mixture was quenched with water (100 mL) and extracted with EtOAc (3×100 mL). The combined organic phases were washed with brine (100 mL), dried over anhydrous Na₂SO₄, filtered and concentrated under reduced pressure. The residue was purified by flash silica gel chromatography (eluting EtOAc in petroleum ether) to afford tert-butyl 1-(6-bromo-2-methylpyridin-3-yl)-1H-1,2,3-triazole-4-carboxylate. LCMS (m/z): 339 (M+H)⁺.

### Step 3. Preparation of methyl 1-(6-chloro-2-methylpyridin-3-yl)-1H-1,2,3-triazole-4-carboxylate

HCl in MeOH (30 mL, 90 mmol, 3 M) was added to a solution of tert-butyl 1-(6-bromo-2-methylpyridin-3-yl)-1H-1,2,3-triazole-4-carboxylate (3.8 g, 11.20 mmol) in MeOH (10 mL) at 25° C. over 5 minutes. After stirring for 16 hours at 25° C. the mixture was concentrated under reduced pressure to afford methyl 1-(6-chloro-2-methylpyridin-3-yl)-1H-1,2,3-triazole-4-carboxylate which was used without further purification. LCMS (m/z): 253 (M+H)⁺.

### Step 4. Preparation of 1-(6-chloro-2-methylpyridin-3-yl)-1H-1,2,3-triazole-4-carboxylic acid

A mixture of methyl 1-(6-chloro-2-methylpyridin-3-yl)-1H-1,2,3-triazole-4-carboxylate (3 g, 11.87 mmol) and lithium hydroxide monohydrate (0.996 g, 23.75 mmol) in THF (15 mL) and water (15 mL) was stirred at 25° C. for 4 hours. The reaction mixture was quenched with water (20 mL), acidified with HCl_{(aq)} (2 M) (pH=1), and extracted with EtOAc (3×50 mL). The combined organic phases were washed with brine (50 mL), dried over anhydrous Na₂SO₄, filtered and concentrated under reduced pressure to afford 1-(6-chloro-2-methylpyridin-3-yl)-1H-1,2,3-triazole-4-carboxylic acid. LCMS (m/z): 239 (M+H)⁺.

### Preparation of Intermediate 1-((1R,3R)-3-hydroxycyclobutyl)-1H-1,2,3-triazole-4-carboxylic acid

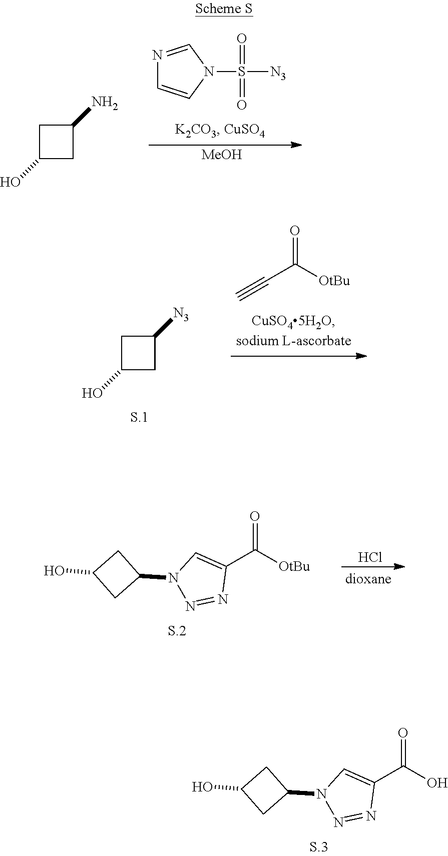

### Step 1. Preparation of (1R,3R)-3-azidocyclobutan-1-ol

A mixture of (1R,3R)-3-aminocyclobutan-1-ol hydrochloride (500 mg, 4.05 mmol), K₂CO₃ (839 mg, 6.07 mmol), copper(II) sulfate (32.3 mg, 0.202 mmol) and 1H-imidazole-1-sulfonyl azide (701 mg, 4.05 mmol) in MeOH (50 mL) was stirred for 16 hour at 25° C. The reaction mixture was quenched with water (50 mL) and extracted with EtOAc (3×100 mL). The combined organic phases were washed with brine (100 mL), dried over anhydrous Na₂SO₄, filtered and concentrated under reduced pressure to afford (1R,3R)-3-aminocyclobutan-1-ol hydrochloride which was used without further purification.

### Step 2. Preparation of tert-butyl 1-((1R,3R)-3-hydroxycyclobutyl)-1H-1,2,3-triazole-4-carboxylate

A solution of (1R,3R)-3-azidocyclobutan-1-ol (250 mg, 2.210 mmol), (+)-sodium 1-ascorbate (43.8 mg, 0.221 mmol), copper(II) sulfate (35.3 mg, 0.221 mmol) and tert-butyl propiolate (558 mg, 4.42 mmol) in t-BuOH (5 mL) and water (0.5 mL) was stirred at 50° C. for 16 hours. The reaction mixture was quenched with water (100 mL) and extracted with EtOAc (3×100 mL). The combined organic phases were washed with brine (100 mL), dried over anhydrous Na₂SO₄, filtered and concentrated under reduced pressure. The residue was purified by silica gel chromatography (eluting ethyl acetate in hexanes) to give afford tert-butyl 1-((1R,3R)-3-hydroxycyclobutyl)-1H-1,2,3-triazole-4-carboxylate. LCMS (ESI) m/z: 184 (M+2H-tBu)⁺

### Step 3. Preparation of 1-((1R,3R)-3-hydroxycyclobutyl)-1H-1,2,3-triazole-4-carboxylic acid

A mixture of tert-butyl 1-((1R,3R)-3-hydroxycyclobutyl)-1H-1,2,3-triazole-4-carboxylate (200 mg, 0.836 mmol) in HCl/dioxane (5 mL) was stirred at 25° C. for 16 hours. The mixture was concentrated under reduced pressure to afford 1-((1R,3R)-3-hydroxycyclobutyl)-1H-1,2,3-triazole-4-carboxylic acid which was used without further purification.

Examples shown in Intermediate Table S below, were or may be prepared according to procedures analogous to those outlined in Scheme S above using the appropriate starting materials, described in the Preparations or Intermediates above, or as obtained from commercial sources.

Intermediate Table S

| | | | Mass |
|---|---|---|---|
| Example | Structure | Name | [M + H]⁺ |
| S.4 | 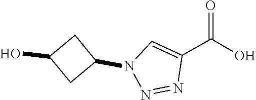 [SMILES: O=C(O)c1cn([C@H]2C[C@@H](O)C2)nn1] | 1-((1S,3S)-3- hydroxycyclobutyl)-1H- 1,2,3-triazole-4-carboxylic acid | 184 |

### Preparation of Intermediate 3-(2,6-dimethylpyridin-3-yl)isoxazole-5-carboxylic acid (S.4

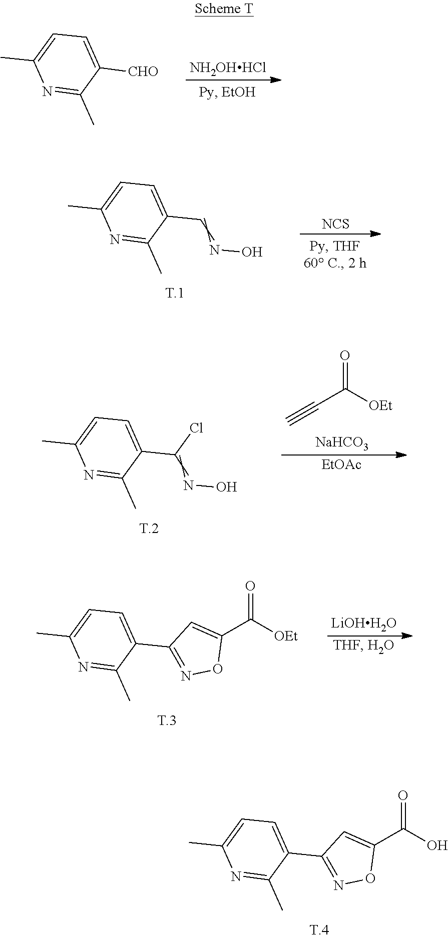

### Step 1. Preparation of 2,6-dimethylnicotinaldehyde oxime

A mixture of hydroxylamine hydrochloride (1.671 g, 24.04 mmol), 2,6-dimethylnicotinaldehyde (2.5 g, 18.50 mmol), pyridine (2.98 mL, 37.0 mmol) in EtOH (30 mL) was stirred for 3 hours at 80° C. The reaction mixture was quenched with water (50 mL) and extracted with EtOAc (3×50 mL). The combined organic phases were washed with brine (30 mL), dried over anhydrous Na₂SO₄, filtered and concentrated under reduced pressure to afford 2,6-dimethylnicotinaldehyde oxime.

### Step 2. Preparation of N-hydroxy-2,6-dimethylnicotinimidoyl chloride

A mixture of 2,6-dimethylnicotinaldehyde oxime (500 mg, 3.33 mmol), NCS (489 mg, 3.66 mmol) and pyridine (0.027 mL, 0.333 mmol) in THF (30 mL) was stirred for 6 hours at 60° C. The mixture was filtered and the filtrate was concentrated under reduced pressure to give the afford N-hydroxy-2,6-dimethylnicotinimidoyl chloride which was used without further purification.

### Step 3. Preparation of ethyl 3-(2,6-dimethylpyridin-3-yl)isoxazole-5-carboxylate

A mixture of N-hydroxy-2,6-dimethylnicotinimidoyl chloride (260 mg, 1.408 mmol)), ethyl propiolate (207 mg, 2.112 mmol) and sodium bicarbonate (237 mg, 2.82 mmol)) in EtOAc (3 mL) was stirred for 2 hours at 100° C. The mixture was filtered and the filtrate was concentrated under reduced pressure. The residue was purified by silica gel chromatography (eluting ethyl acetate in petroleum ether) to afford ethyl 3-(2,6-dimethylpyridin-3-yl)isoxazole-5-carboxylate. LCMS (ESI) m/z: 247 (M+H)⁺.

### Step 4. Preparation of 3-(2,6-dimethylpyridin-3-yl)isoxazole-5-carboxylic acid

A mixture of ethyl 3-(2,6-dimethylpyridin-3-yl)isoxazole-5-carboxylate (300 mg, 1.218 mmol) and lithium hydroxide hydrate (102 mg, 2.436 mmol) in THF (3 mL) was stirring for 1 hour at 25° C. The mixture was adjust to pH=3 and then purified via reverse phase HPLC (eluting acetonitrile in water with 0.1% TFA) to afford 3-(2,6-dimethylpyridin-3-yl)isoxazole-5-carboxylic acid. ¹H NMR (MeOD, 400 MHz) δ 8.61 (d, J=8.11 Hz, 1H), 7.81 (d, J=8.23 Hz, 1H), 7.54 (s, 1H), 2.94 (s, 3H), 2.82 (s, 3H)

### Preparation of Intermediate 1-(1H-pyrazol-4-yl)-1H-1,2,3-triazole-4-carboxylic acid (U.3

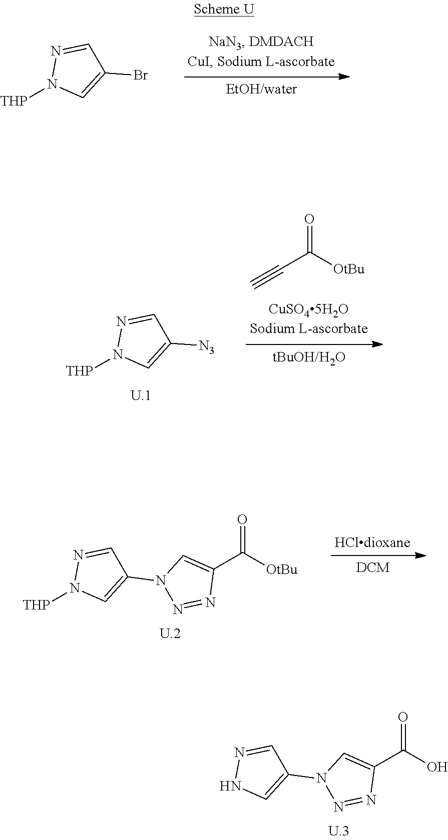

### Step 1. Preparation of 4-azido-1-(tetrahydro-2H-pyran-2-yl)-1H-pyrazole

A mixture of 4-bromo-1-(tetrahydro-2H-pyran-2-yl)-1H-pyrazole (2 g, 8.65 mmol), copper(I) iodide (0.165 g, 0.865 mmol), (+)-sodium L-ascorbate (0.086 g, 0.433 mmol), (1R,2R)—N,N′-dimethyl-1,2-cyclohexanediamine (0.185 g, 1.298 mmol) and NaN₃ (0.830 g, 12.77 mmol) in Ethanol/H₂O=7:3 (20 mL) were charged in a vial and the mixture was degassed and backfilled with N₂ (three times). The mixture was heated to 80° C. for 16 hours. After cooling to room temperature the reaction was quenched by the addition of NaOH solution (1M) bringing the pH>9 and the mixture was extracted with EtOAc (3×40 mL). The combined organic phases were washed with brine (30 mL), dried over anhydrous Na2SO4, filtered and concentrated under reduced pressure. The residue was purified by silica gel chromatography (eluting ethyl acetate in petroleum ether) to afford 4-azido-1-(tetrahydro-2H-pyran-2-yl)-1H-pyrazole. LCMS (ESI) m/z: 166 [M+H−N2]⁺.

### Step 2. Preparation of tert-butyl 1-(1-(tetrahydro-2H-pyran-2-yl)-1H-pyrazol-4-yl)-1H-1,2,3-triazole-4-carboxylate

A solution of tert-butyl but-3-ynoate (229 mg, 1.630 mmol), 4-azido-1-(tetrahydro-2H-pyran-2-yl)-1H-pyrazole (300 mg, 1.553 mmol), copper(II) sulfate (24.78 mg, 0.155 mmol) and (+)-sodium L-ascorbate (30.8 mg, 0.155 mmol) in t-BuOH (3 mL) and water (0.300 mL) was heated to 50° C. and stirred for 2 hours. The reaction mixture was quenched with NH₄OH (15 mL) and extracted with EtOAc (3×40 mL). The combined organic phases were washed with brine (50 mL), dried over anhydrous Na₂SO₄, filtered and concentrated under reduced pressure. The residue was purified by silica gel chromatography (eluting ethyl acetate in petroleum ether) to afford tert-butyl 1-(1-(tetrahydro-2H-pyran-2-yl)-1H-pyrazol-4-yl)-1H-1,2,3-triazole-4-carboxylate. LCMS (ESI) m/z: 264 [M+2H-tBu]⁺

### Step 3. Preparation of 1-(1H-pyrazol-4-yl)-1H-1,2,3-triazole-4-carboxylic acid

A mixture of tert-butyl 1-(1-(tetrahydro-2H-pyran-2-yl)-1H-pyrazol-4-yl)-1H-1,2,3-triazole-4-carboxylate (260 mg, 0.814 mmol) and hydrogen chloride in dioxane (2 mL, 8.00 mmol) in DCM (10 mL) was stirred at 25° C. for 1 hour. The solvent was removed under vacuum to afford 1-(1H-pyrazol-4-yl)-1H-1,2,3-triazole-4-carboxylic acid which was used without further purification. LCMS (ESI) m/z: 180 (M+H)⁺.

### Preparation of Intermediate 5-fluoro-1-methyl-1H-1,2,3-triazole-4-carboxylic acid (V.3

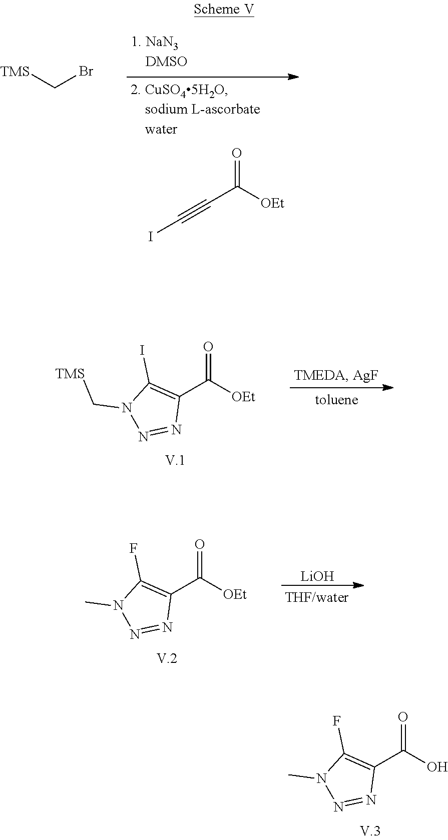

### Step 1. Preparation of ethyl 5-iodo-1-((trimethylsilyl)methyl)-1H-1,2,3-triazole-4-carboxylate

Sodium azide (1.53 g, 23.53 mmol) was added to a solution of (bromomethyl)trimethylsilane (3 g, 18 mmol) in DMSO (30 mL) at 25° C. over 3 minutes. After stirring for 48 hours at 80° C. the reaction was cooled to 25° C. and ethyl 3-iodopropiolate (4.83 g, 21.54 mmol), copper (II) sulfate pentahydrate (0.287 g, 1.795 mmol) and a solution of (+)-sodium L-ascorbate (0.356 g, 1.795 mmol) were added and the mixture was stirred for another 16 hours at 50° C. The mixture was adjust pH>9 with 2M NaOH solution and extracted with EtOAc (3×50 mL). The combined organic phases were washed with brine (50 mL), dried over anhydrous Na₂SO₄, filtered and concentrated under reduced pressure. The residue was purified by flash silica gel chromatography (eluting ethyl acetate in petroleum ether) to afford ethyl 5-iodo-1-((trimethylsilyl)methyl)-1H-1,2,3-triazole-4-carboxylate. LCMS (ESI) m/z: 354 (M+H)⁺.

### Step 2. Preparation of ethyl 5-fluoro-1-methyl-1H-1,2,3-triazole-4-carboxylate

A mixture of ethyl 5-iodo-1-((trimethylsilyl)methyl)-1H-1,2,3-triazole-4-carboxylate (640 mg, 1.812 mmol), TMEDA (0.137 mL, 0.906 mmol) and silver(I) fluoride (1149 mg, 9.06 mmol) in toluene (15 mL) was stirred at 120° C. for 16 hours. The mixture was filtered and the filtrate was purified via reverse phase HPLC (eluting acetonitrile in water with 0.1% TFA modifier) to afford ethyl 5-fluoro-1-methyl-1H-1,2,3-triazole-4-carboxylate. LCMS (ESI) m/z: 174 (M+H)⁺.

### Step 3. Preparation of 5-fluoro-1-methyl-1H-1,2,3-triazole-4-carboxylic acid

LiOH·H₂O (10.91 mg, 0.260 mmol) was added to a solution of ethyl 5-fluoro-1-methyl-1H-1,2,3-triazole-4-carboxylate (30 mg, 0.173 mmol) in THF:water=5:1 (1 mL) at 25° C. over 1 minute. After stirring for 1 hour at 25° C. HCl in dioxane (0.5 mL, 4 M) was added and the solvent was removed under vacuum to afford 5-fluoro-1-methyl-1H-1,2,3-triazole-4-carboxylic acid which was used in the next step without further purification. LCMS (ESI) m/z: 146 (M+H)⁺.

### Preparation of Intermediate 1-(6-phenylpyridazin-3-yl)ethanamine (W.3

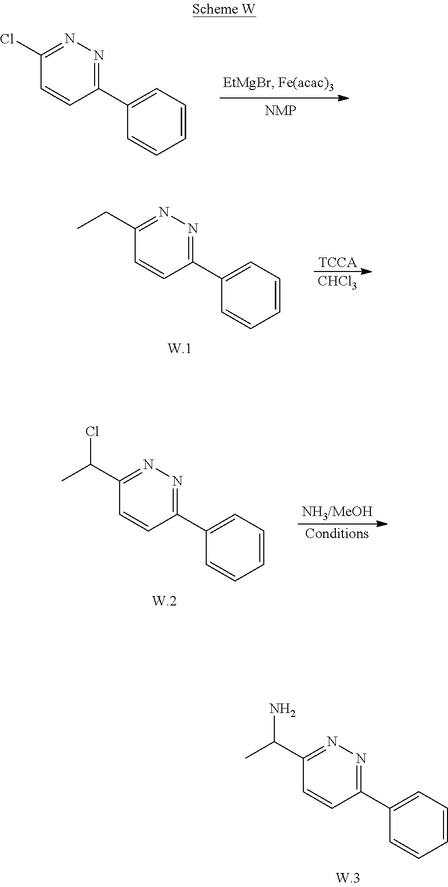

### Step 1. Preparation of 3-ethyl-6-phenylpyridazine

Ferric acetylacetonate (0.185 g, 0.525 mmol) was added to a solution of 3-chloro-6-phenylpyridazine (1 g, 5 mmol) in THF (20 mL) and NMP (2 mL) was added at 0° C. over 1 minute. After stirring for 2 minutes at 0° C., ethylmagnesium bromide (2 mL, 6 mmol) was added to the mixture at 0° C. The resulting mixture was stirred for another 16 hours at 25° C. The reaction mixture was quenched with NH₄Cl aqueous solution (20 mL) and extracted with EtOAc (3×30 mL). The combined organic phases were washed with brine (20 mL), dried over anhydrous Na₂SO₄, filtered and concentrated under reduced pressure. The residue was purified by silica gel chromatography (eluting ethyl acetate in petroleum ether) to afford 3-ethyl-6-phenylpyridazine. LCMS (ESI) m/z: 185 (M+H)⁺.

### Step 2. Preparation of 3-(1-chloroethyl)-6-phenylpyridazine

A mixture of 3-ethyl-6-phenylpyridazine (640 mg, 3.47 mmol) and trichloroisocyanuric acid (323 mg, 1.389 mmol) in CHCl₃ (30 mL) was stirred at 60° C. for 4 hours. The reaction mixture was quenched with water (20 mL) and extracted with EtOAc (3×20 mL). The combined organic phases were washed with brine (15 mL), dried over anhydrous Na₂SO₄, filtered and concentrated under reduced pressure. The residue was purified by silica gel chromatography (eluting ethyl acetate in petroleum ether) to afford 3-(1-chloroethyl)-6-phenylpyridazine. LCMS (ESI) m/z: 219.0 (M+H)⁺.

### Step 3: Preparation of 1-(6-phenylpyridazin-3-yl)ethanamine

A mixture of 3-(1-chloroethyl)-6-phenylpyridazine (190 mg, 0.869 mmol) in NH₃ in MeOH (7 mL, 7 mol) was stirred at 80° C. for 48 hours. The mixture was filtered and the filtrate was purified via reverse phase HPLC (eluting acetonitrile in water with 0.04% HCl modifier) to afford 1-(6-phenylpyridazin-3-yl)ethan-1-amine. LCMS (ESI) m/z: 200 (M+H)⁺.

### Example 1

### Preparation of 1-(2,6-dimethylpyridin-3-yl)-N-((5-phenyl-1,3,4-thiadiazol-2-yl)methyl)-1H-1,2,3-triazole-4-carboxamide

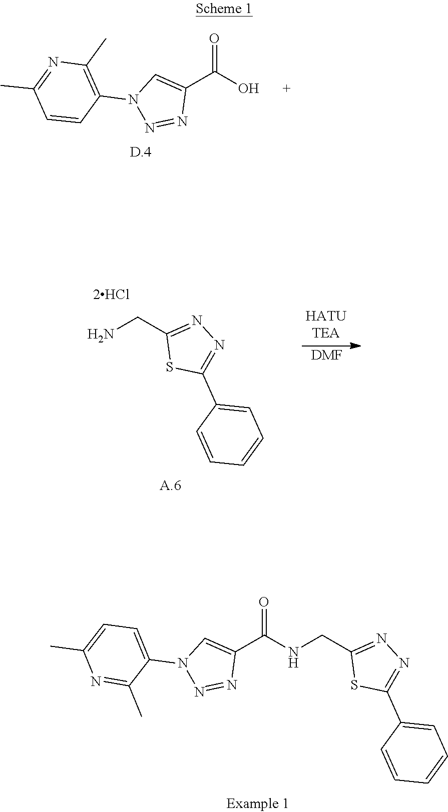

In a 384 well plate, a solution of HATU (15.6 μL, 7.8 μmol, 0.5 M in DMF) in DMF was added to a solution of 1-(2,6-dimethylpyridin-3-yl)-1H-1,2,3-triazole-4-carboxylic acid (29.4 μL, 7.2 μmol, 0.254 M in DMF) in DMF and aged at room temperature for 20 minutes. After which time TEA (1.67 μL 12 μmol) was added to a solution of (5-phenyl-1,3,4-thiadiazol-2-yl)methanamine dihydrochloride (13.33 μL, 6 μmol, 0.4 M in DMF) in DMF. This solution was added to the above, pre-aged, activated carboxylic acid. The 384-well plate containing 60 μL of total reaction volume was sealed and the plate was at shaken at 700 rmp at 25° C. for 16 hours. The reaction quenched with 40 μL of a DMSO/water (9:1) solution generating a 60 mM solution. 1 μL of this solution was diluted to 30 μL with DMSO to prepare a 2 mM stock solution for “direct to biology” (DtB) assay where potency is measured on the crude reaction mixture. Then 0.833 μL of the above, quenched stock was diluted to 100 μL with DMSO for analytical UPLC-MS analysis. The remaining 98.17 μL was sent for HPLC purification (mass directed) to afford (5-phenyl-1,3,4-thiadiazol-2-yl)methyl 1-(2,6-dimethylpyridin-3-yl)-1H-1,2,3-triazole-4-carboxylate. The purified compound is then submitted to the biochemical assay. LC/MS (m/z): 392 (M+H)⁺. ¹H NMR (600 MHz, DMSO) δ 9.71 (t, J=6.0 Hz, 1H), 9.09 (s, 1H), 7.98-7.95 (m, 2H), 7.85 (d, J=8.1 Hz, 1H), 7.58-7.52 (m, 3H), 7.35 (d, J=8.1 Hz, 1H), 4.92 (d, J=6.1 Hz, 2H), 2.55 (s, 3H), 2.33 (s, 3H).

Examples shown in Example Table 1 below, were prepared according to procedures analogous to those outlined in Example 1 and general Scheme 2 above using the appropriate starting materials obtained from methods described above or elsewhere, or as obtained from commercial sources.

Example Table 1

| | | | Mass |
|---|---|---|---|
| Example | Structure | Name | [M + H]⁺ |
| Example 2 | 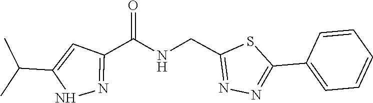 [SMILES: CC(C)c1cc(C(=O)NCc2nnc(-c3ccccc3)s2)n[nH]1] | 5-isopropyl-N-[(5-phenyl-1,3,4- thiadiazol-2-yl)methyl]-1H- pyrazole-3-carboxamide | 328 |
| Example 3 | 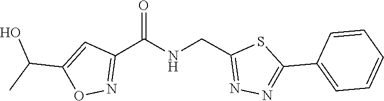 [SMILES: CC(O)c1cc(C(=O)NCc2nnc(-c3ccccc3)s2)no1] | 5-(1-hydroxyethyl)-N-[(5- phenyl-1,3,4-thiadiazol-2- yl)methyl]isoxazole-3- carboxamide | 331 |
| Example 4 | 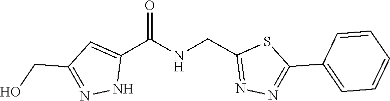 [SMILES: O=C(NCc1nnc(-c2ccccc2)s1)c1cc(CO)n[nH]1] | 3-(hydroxymethyl)-N-((5- phenyl-1,3,4-thiadiazol-2- yl)methyl)-1H-pyrazole-5- carboxamide | 316 |
| Example 5 | 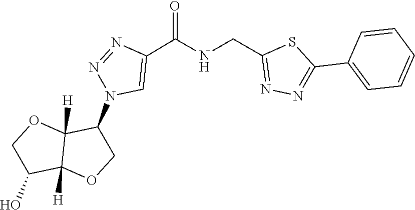 [SMILES: O=C(NCc1nnc(-c2ccccc2)s1)c1cn([C@H]2CO[C@H]3[C@@H]2OC[C@H]3O)nn1] | 1-[(3S,3aR,6R,6aR)-6-hydroxy- 2,3,3a,5,6,6a- hexahydrofuro[3,2-b]furan-3- yl]-N-[(5-phenyl-1,3,4- thiadiazol-2-yl)methyl]triazole- 4-carboxamide | 415 |
| Example 6 | 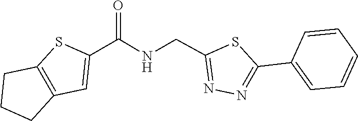 [SMILES: O=C(NCc1nnc(-c2ccccc2)s1)c1cc2c(s1)CCC2] | N-[(5-phenyl-1,3,4-thiadiazol- 2-yl)methyl]-5,6-dihydro-4H- cyclopenta[b]thiophene-2- carboxamide | 342 |
| Example 7 | 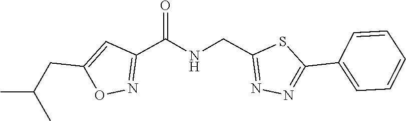 [SMILES: CC(C)Cc1cc(C(=O)NCc2nnc(-c3ccccc3)s2)no1] | 5-isobutyl-N-[(5-phenyl-1,3,4- thiadiazol-2- yl)methyl]isoxazole-3- carboxamide | 343 |
| Example 8 | 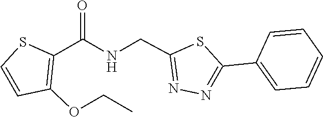 [SMILES: CCOc1ccsc1C(=O)NCc1nnc(-c2ccccc2)s1] | 3-ethoxy-N-[(5-phenyl-1,3,4- thiadiazol-2- yl)methyl]thiophene-2- carboxamide | 346 |
| Example 9 | 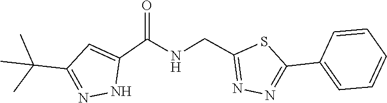 [SMILES: CC(C)(C)c1cc(C(=O)NCc2nnc(-c3ccccc3)s2)[nH]n1] | 3-(tert-butyl)-N-((5-phenyl- 1,3,4-thiadiazol-2-yl)methyl)- 1H-pyrazole-5-carboxamide | 342 |
| Example 10 | 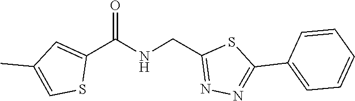 [SMILES: Cc1csc(C(=O)NCc2nnc(-c3ccccc3)s2)c1] | 4-methyl-N-[(5-phenyl-1,3,4- thiadiazol-2- yl)methyl]thiophene-2- carboxamide | 316 |
| Example 11 | 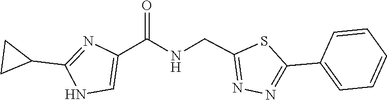 [SMILES: O=C(NCc1nnc(-c2ccccc2)s1)c1c[nH]c(C2CC2)n1] | 2-cyclopropyl-N-((5-phenyl- 1,3,4-thiadiazol-2- yl)methyl)-1H-imidazole-4- carboxamide | 326 |
| Example 12 | 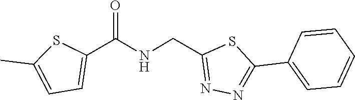 [SMILES: Cc1ccc(C(=O)NCc2nnc(-c3ccccc3)s2)s1] | 5-methyl-N-[(5-phenyl-1,3,4- thiadiazol-2- yl)methyl]thiophene-2- carboxamide | 316 |
| Example 13 | 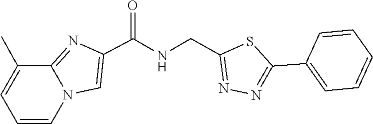 [SMILES: Cc1cccn2cc(C(=O)NCc3nnc(-c4ccccc4)s3)nc12] | 8-methyl-N-((5-phenyl-1,3,4- thiadiazol-2- yl)methyl)imidazo[1,2- a]pyridine-2-carboxamide | 350 |
| Example 14 | 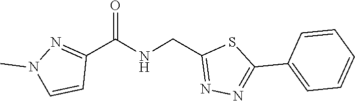 [SMILES: Cn1ccc(C(=O)NCc2nnc(-c3ccccc3)s2)n1] | 1-methyl-N-[(5-phenyl-1,3,4- thiadiazol-2- yl)methyl]pyrazole-3- carboxamide | 300 |
| Example 15 | 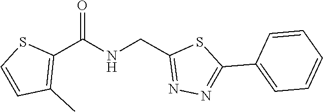 [SMILES: Cc1ccsc1C(=O)NCc1nnc(-c2ccccc2)s1] | 3-methyl-N-[(5-phenyl-1,3,4- thiadiazol-2- yl)methyl]thiophene-2- carboxamide | 316 |
| Example 16 | 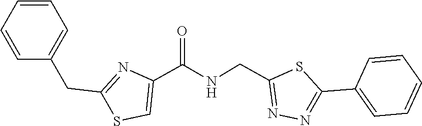 [SMILES: O=C(NCc1nnc(-c2ccccc2)s1)c1csc(Cc2ccccc2)n1] | 2-benzyl-N-((5-phenyl-1,3,4- thiadiazol-2- yl)methyl)thiazole-4- carboxamide | 393 |
| Example 17 | 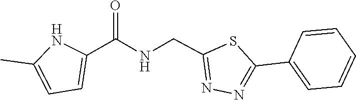 [SMILES: Cc1ccc(C(=O)NCc2nnc(-c3ccccc3)s2)[nH]1] | 5-methyl-N-[(5-phenyl-1,3,4- thiadiazol-2-yl)methyl]-1H- pyrrole-2-carboxamide | 299 |
| Example 18 | 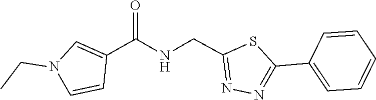 [SMILES: CCn1ccc(C(=O)NCc2nnc(-c3ccccc3)s2)c1] | 1-ethyl-N-[(5-phenyl-1,3,4- thiadiazol-2-yl)methyl]pyrrole- 3-carboxamide | 313 |
| Example 19 | 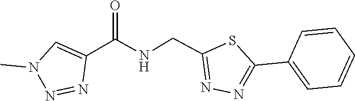 [SMILES: Cn1cc(C(=O)NCc2nnc(-c3ccccc3)s2)nn1] | 1-methyl-N-[(5-phenyl-1,3,4- thiadiazol-2-yl)methyl]triazole- 4-carboxamide | 301 |
| Example 20 | 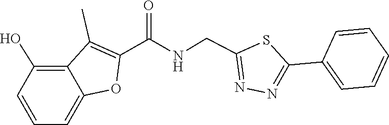 [SMILES: Cc1c(C(=O)NCc2nnc(-c3ccccc3)s2)oc2cccc(O)c12] | 4-hydroxy-3-methyl-N-[(5- phenyl-1,3,4-thiadiazol-2- yl)methyl]benzofuran-2- carboxamide | 366 |
| Example 21 | 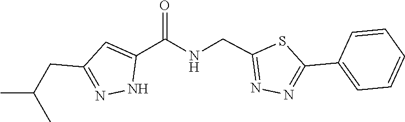 [SMILES: CC(C)Cc1cc(C(=O)NCc2nnc(-c3ccccc3)s2)[nH]n1] | 3-isobutyl-N-((5-phenyl-1,3,4- thiadiazol-2-yl)methyl)-1H- pyrazole-5-carboxamide | 342 (456) |
| Example 22 | 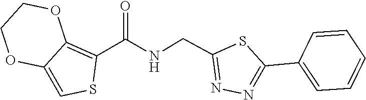 [SMILES: O=C(NCc1nnc(-c2ccccc2)s1)c1scc2c1OCCO2] | N-[(5-phenyl-1,3,4-thiadiazol- 2-yl)methyl]-2,3- dihydrothieno[3,4- b][1,4]dioxine-5-carboxamide | 360 |
| Example 23 | 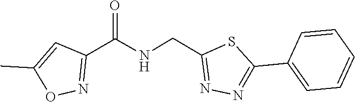 [SMILES: Cc1cc(C(=O)NCc2nnc(-c3ccccc3)s2)no1] | 5-methyl-N-[(5-phenyl-1,3,4- thiadiazol-2- yl)methyl]isoxazole-3- carboxamide | 301 |
| Example 24 | 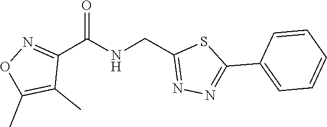 [SMILES: Cc1onc(C(=O)NCc2nnc(-c3ccccc3)s2)c1C] | 4,5-dimethyl-N-[(5-phenyl- 1,3,4-thiadiazol-2- yl)methyl]isoxazole-3- carboxamide | 315 |
| Example 25 | 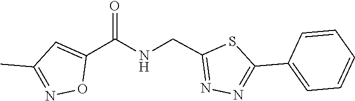 [SMILES: Cc1cc(C(=O)NCc2nnc(-c3ccccc3)s2)on1] | 3-methyl-N-[(5-phenyl-1,3,4- thiadiazol-2- yl)methyl]isoxazole-5- carboxamide | 301 |
| Example 26 | 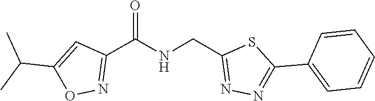 [SMILES: CC(C)c1cc(C(=O)NCc2nnc(-c3ccccc3)s2)no1] | 5-isopropyl-N-[(5-phenyl-1,3,4- thiadiazol-2- yl)methyl]isoxazole-3- carboxamide | 329 |
| Example 27 | 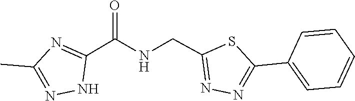 [SMILES: Cc1n[nH]c(C(=O)NCc2nnc(-c3ccccc3)s2)n1] | 3-methyl-N-((5-phenyl-1,3,4- thiadiazol-2-yl)methyl)-1H- 1,2,4-triazole-5-carboxamide | 301 |
| Example 28 | 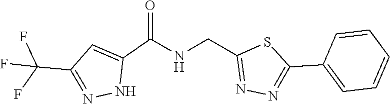 [SMILES: O=C(NCc1nnc(-c2ccccc2)s1)c1cc(C(F)(F)F)n[nH]1] | N-[(5-phenyl-1,3,4-thiadiazol- 2-yl)methyl]-3- (trifluoromethyl)-1H-pyrazole- 5-carboxamide | 354 |
| Example 29 | 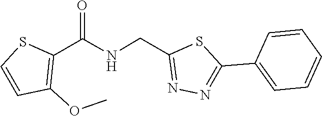 [SMILES: COc1ccsc1C(=O)NCc1nnc(-c2ccccc2)s1] | 3-methoxy-N-[(5-phenyl-1,3,4- thiadiazol-2- yl)methyl]thiophene-2- carboxamide | 332 |
| Example 30 | 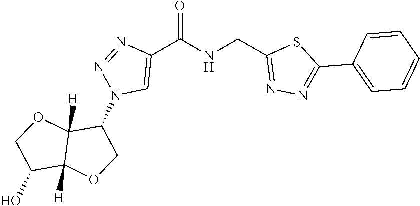 [SMILES: O=C(NCc1nnc(-c2ccccc2)s1)c1cn([C@@H]2CO[C@H]3[C@@H]2OC[C@H]3O)nn1] | 1-((3R,3aR,6R,6aR)-6- hydroxyhexahydrofuro[3,2- b]furan-3-yl)-N-((5-phenyl- 1,3,4-thiadiazol-2-yl)methyl)- 1H-1,2,3-triazole-4- carboxamide | 416 |
| Example 31 | 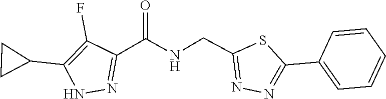 [SMILES: O=C(NCc1nnc(-c2ccccc2)s1)c1n[nH]c(C2CC2)c1F] | 5-cyclopropyl-4-fluoro-N-[(5- phenyl-1,3,4-thiadiazol-2- yl)methyl]-1H-pyrazole-3- carboxamide | 344 |
| Example 32 | 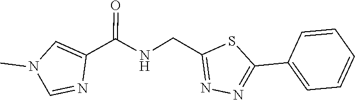 [SMILES: Cn1cnc(C(=O)NCc2nnc(-c3ccccc3)s2)c1] | 1-methyl-N-((5-phenyl-1,3,4- thiadiazol-2-yl)methyl)-1H- imidazole-4-carboxamide | 300 |
| Example 33 | 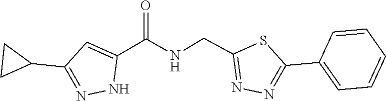 [SMILES: O=C(NCc1nnc(-c2ccccc2)s1)c1cc(C2CC2)n[nH]1] | 3-cyclopropyl-N-[(5-phenyl- 1,3,4-thiadiazol-2-yl)methyl]- 1H-pyrazole-5-carboxamide | 326 |
| Example 34 | 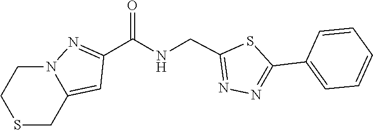 [SMILES: O=C(NCc1nnc(-c2ccccc2)s1)c1cc2n(n1)CCSC2] | N-[(5-phenyl-1,3,4-thiadiazol- 2-yl)methyl]-6,7-dihydro-4H- pyrazolo[5,1-c][1,4]thiazine-2- carboxamide | 358 |
| Example 35 | 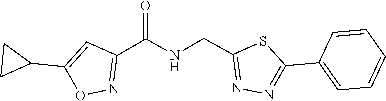 [SMILES: O=C(NCc1nnc(-c2ccccc2)s1)c1cc(C2CC2)on1] | 5-cyclopropyl-N-[(5-phenyl- 1,3,4-thiadiazol-2- yl)methyl]isoxazole-3- carboxamide | 327 |
| Example 36 | 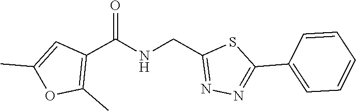 [SMILES: Cc1cc(C(=O)NCc2nnc(-c3ccccc3)s2)c(C)o1] | 2,5-dimethyl-N-[(5-phenyl- 1,3,4-thiadiazol-2- yl)methyl]furan-3-carboxamide | 314 |
| Example 37 | 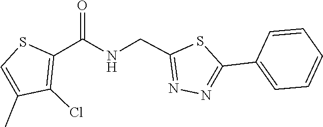 [SMILES: Cc1csc(C(=O)NCc2nnc(-c3ccccc3)s2)c1Cl] | 3-chloro-4-methyl-N-[(5- phenyl-1,3,4-thiadiazol-2- yl)methyl]thiophene-2- carboxamide | 350 |
| Example 38 | 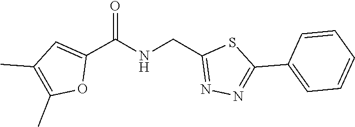 [SMILES: Cc1cc(C(=O)NCc2nnc(-c3ccccc3)s2)oc1C] | 4,5-dimethyl-N-[(5-phenyl- 1,3,4-thiadiazol-2- yl)methyl]furan-2-carboxamide | 314 |
| Example 39 | 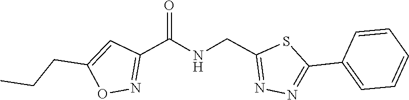 [SMILES: CCCc1cc(C(=O)NCc2nnc(-c3ccccc3)s2)no1] | N-[(5-phenyl-1,3,4-thiadiazol- 2-yl)methyl]-5-propyl- isoxazole-3-carboxamide | 329 |
| Example 40 | 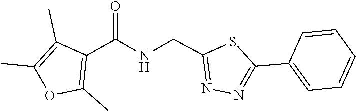 [SMILES: Cc1oc(C)c(C(=O)NCc2nnc(-c3ccccc3)s2)c1C] | 2,4,5-trimethyl-N-[(5-phenyl- 1,3,4-thiadiazol-2- yl)methyl]furan-3-carboxamide | 328 |
| Example 41 | 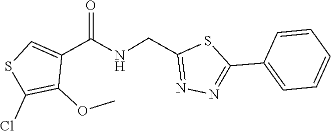 [SMILES: COc1c(C(=O)NCc2nnc(-c3ccccc3)s2)csc1Cl] | 5-chloro-4-methoxy-N-[(5- phenyl-1,3,4-thiadiazol-2- yl)methyl]thiophene-3- carboxamide | 366 |
| Example 42 | 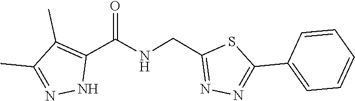 [SMILES: Cc1n[nH]c(C(=O)NCc2nnc(-c3ccccc3)s2)c1C] | 3,4-dimethyl-N-((5-phenyl- 1,3,4-thiadiazol-2- yl)methyl)-1H-pyrazole-5- carboxamide | 314 |
| Example 43 | 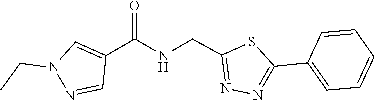 [SMILES: CCn1cc(C(=O)NCc2nnc(-c3ccccc3)s2)cn1] | 1-ethyl-N-[(5-phenyl-1,3,4- thiadiazol-2- yl)methyl]pyrazole-4- carboxamide | 314 |
| Example 44 | 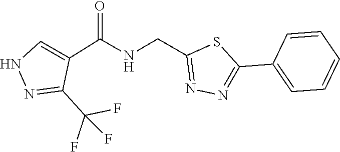 [SMILES: O=C(NCc1nnc(-c2ccccc2)s1)c1c[nH]nc1C(F)(F)F] | N-[(5-phenyl-1,3,4-thiadiazol- 2-yl)methyl]-3- (trifluoromethyl)-1H-pyrazole- 4-carboxamide | 354 |
| Example 45 | 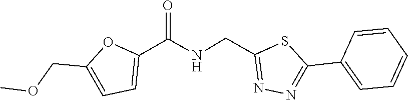 [SMILES: COCc1ccc(C(=O)NCc2nnc(-c3ccccc3)s2)o1] | 5-(methoxymethyl)-N-[(5- phenyl-1,3,4-thiadiazol-2- yl)methyl]furan-2-carboxamide | 330 |
| Example 46 | 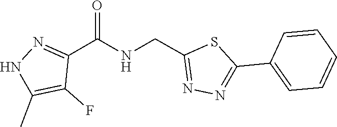 [SMILES: Cc1[nH]nc(C(=O)NCc2nnc(-c3ccccc3)s2)c1F] | 4-fluoro-5-methyl-N-[(5- phenyl-1,3,4-thiadiazol-2- yl)methyl]-1H-pyrazole-3- carboxamide | 318 |
| Example 47 | 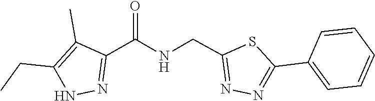 [SMILES: CCc1[nH]nc(C(=O)NCc2nnc(-c3ccccc3)s2)c1C] | 5-ethyl-4-methyl-N-((5- phenyl-1,3,4-thiadiazol-2- yl)methyl)-1H-pyrazole-3- carboxamide | 328 |
| Example 48 | 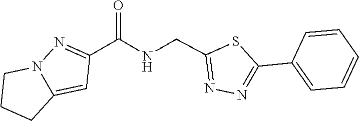 [SMILES: O=C(NCc1nnc(-c2ccccc2)s1)c1cc2n(n1)CCC2] | N-[(5-phenyl-1,3,4-thiadiazol- 2-yl)methyl]-5,6-dihydro-4H- pyrrolo[1,2-b]pyrazole-2- carboxamide | 326 |
| Example 49 | 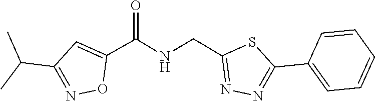 [SMILES: CC(C)c1cc(C(=O)NCc2nnc(-c3ccccc3)s2)on1] | 3-isopropyl-N-[(5-phenyl-1,3,4- thiadiazol-2- yl)methyl]isoxazole-5- carboxamide | 329 |
| Example 50 | 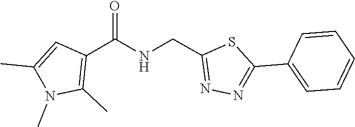 [SMILES: Cc1cc(C(=O)NCc2nnc(-c3ccccc3)s2)c(C)n1C] | 1,2,5-trimethyl-N-[(5-phenyl- 1,3,4-thiadiazol-2- yl)methyl]pyrrole-3- carboxamide | 327 |
| Example 51 | 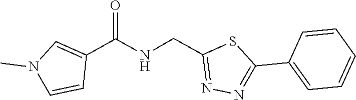 [SMILES: Cn1ccc(C(=O)NCc2nnc(-c3ccccc3)s2)c1] | 1-methyl-N-[(5-phenyl-1,3,4- thiadiazol-2-yl)methyl]pyrrole- 3-carboxamide | 299 |
| Example 52 | 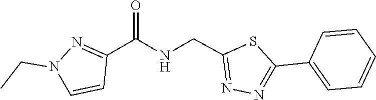 [SMILES: CCn1ccc(C(=O)NCc2nnc(-c3ccccc3)s2)n1] | 1-ethyl-N-[(5-phenyl-1,3,4- thiadiazol-2- yl)methyl]pyrazole-3- carboxamide | 314 |
| Example 53 | 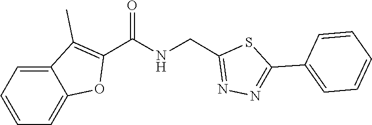 [SMILES: Cc1c(C(=O)NCc2nnc(-c3ccccc3)s2)oc2ccccc12] | 3-methyl-N-[(5-phenyl-1,3,4- thiadiazol-2- yl)methyl]benzofuran-2- carboxamide | 350 |
| Example 54 | 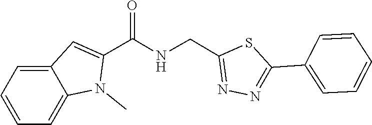 [SMILES: Cn1c(C(=O)NCc2nnc(-c3ccccc3)s2)cc2ccccc21] | 1-methyl-N-((5-phenyl-1,3,4- thiadiazol-2-yl)methyl)-1H- indole-2-carboxamide | 349 |
| Example 55 | 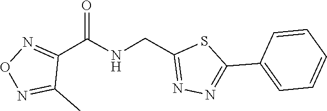 [SMILES: Cc1nonc1C(=O)NCc1nnc(-c2ccccc2)s1] | 4-methyl-N-[(5-phenyl-1,3,4- thiadiazol-2-yl)methyl]-1,2,5- oxadiazole-3-carboxamide | 302 |
| Example 56 | 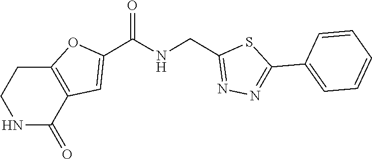 [SMILES: O=C(NCc1nnc(-c2ccccc2)s1)c1cc2c(o1)CCNC2=O] | 4-oxo-N-[(5-phenyl-1,3,4- thiadiazol-2-yl)methyl]-6,7- dihydro-5H-furo[3,2- c]pyridine-2-carboxamide | 355 |
| Example 57 | 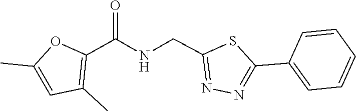 [SMILES: Cc1cc(C)c(C(=O)NCc2nnc(-c3ccccc3)s2)o1] | 3,5-dimethyl-N-[(5-phenyl- 1,3,4-thiadiazol-2- yl)methyl]furan-2-carboxamide | 314 |
| Example 58 | 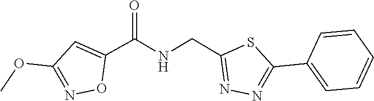 [SMILES: COc1cc(C(=O)NCc2nnc(-c3ccccc3)s2)on1] | 3-methoxy-N-[(5-phenyl-1,3,4- thiadiazol-2- yl)methyl]isoxazole-5- carboxamide | 317 |
| Example 59 | 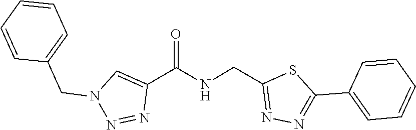 [SMILES: O=C(NCc1nnc(-c2ccccc2)s1)c1cn(Cc2ccccc2)nn1] | 1-benzyl-N-[(5-phenyl-1,3,4- thiadiazol-2-yl)methyl]triazole- 4-carboxamide | 377 |

### Example 60

### Preparation of N-((5-phenyl-1,3,4-thiadiazol-2-yl)methyl)-1-(2,2,2-trifluoroethyl)-1H-1,2,3-triazole-4-carboxamide

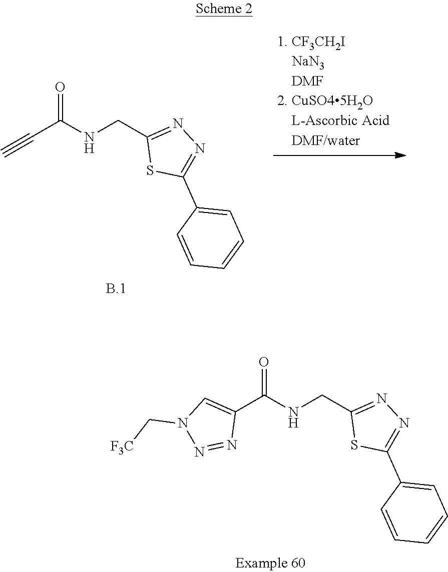 [SMILES: C#CC(=O)NCc1nnc(-c2ccccc2)s1 ; O=C(NCc1nnc(-c2ccccc2)s1)c1cn(CC(F)(F)F)nn1]

In a 4 mL vial with stir bar, screw cap and septum, sodium azide (33 mg, 0.5 mmol) was added to a solution of 1,1,1-trifluoro-2-iodoethane (105 mg, 0.5 mmol) in DMF (0.25 mL) and the reaction was heated to 60° C. for 24 h. Then a solution of N-((5-phenyl-1,3,4-thiadiazol-2-yl)methyl)propiolamide (24.3 mg, 0.1 mmol) in DMF (0.25 mL) was added followed by aqueous solutions of copper(II) sulfate pentahydrate (0.125 mL, 0.01 mmol, 0.08 M in distilled water) and L-ascorbic acid (0.125 mL, 0.02 mmol, 0.16 M in distilled water). The reaction was stirred overnight at room temperature. The reaction was diluted with DMF (2 mL), filtered to remove solids and the residue was purified via reverse phase HPLC (eluting acetonitrile in water, with TFA modifier) to afford N-((5-phenyl-1,3,4-thiadiazol-2-yl)methyl)-1-(2,2,2-trifluoroethyl)-1H-1,2,3-triazole-4-carboxamide. LC/MS (m/z): 369 (M+H)⁺. ¹H NMR (499 MHz, DMSO) δ 9.73-9.61 (m, 1H), 8.80 (s, 1H), 8.02-7.88 (m, 2H), 7.62-7.46 (m, 3H), 5.70-5.53 (m, 2H), 4.88 (d, J=5.4 Hz, 2H).

Examples shown in Example Table 2 below, were prepared according to procedures analogous to those outlined in Example 60 and General Scheme 3 above using the appropriate starting materials obtained from methods described above or elsewhere, or as obtained from commercial sources.

EXAMPLE TABLE 2

| Example | Structure | Name | Mass [M + H]⁺ |
|---|---|---|---|
| Example 61 | 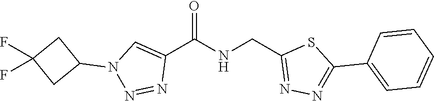 [SMILES: O=C(NCc1nnc(-c2ccccc2)s1)c1cn(C2CC(F)(F)C2)nn1] | 1-(3,3-difluorocyclobutyl)- N-((5-phenyl-1,3,4- thiadiazol-2-yl)methyl)-1H- 1,2,3-triazole-4- carboxamide | 377.1 |
| Example 62 | 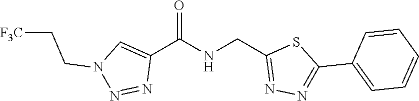 [SMILES: O=C(NCc1nnc(-c2ccccc2)s1)c1cn(CCC(F)(F)F)nn1] | N-((5-phenyl-1,3,4- thiadiazol-2-yl)methyl)-1- (3,3,3-trifluoropropyl)-1H- 1,2,3-triazole-4- carboxamide | 383 |
| Example 63 | 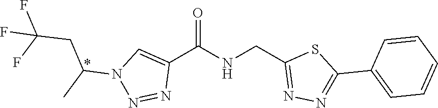 [SMILES: CC(CC(F)(F)F)n1cc(C(=O)NCc2nnc(-c3ccccc3)s2)nn1] | N-((5-phenyl-1,3,4- thiadiazol-2-yl)methyl)-1- (4,4,4-trifluorobutan-2-yl)- 1H-1,2,3-triazole-4- carboxamide, peak 2 | 397 |
| Example 64 | 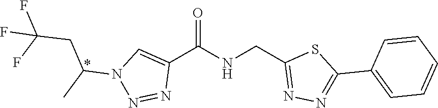 [SMILES: CC(CC(F)(F)F)n1cc(C(=O)NCc2nnc(-c3ccccc3)s2)nn1] | N-((5-phenyl-1,3,4- thiadiazol-2-yl)methyl)-1- (4,4,4-trifluorobutan-2-yl)- 1H-1,2,3-triazole-4- carboxamide, peak 1 | 397 |
| Example 65 | 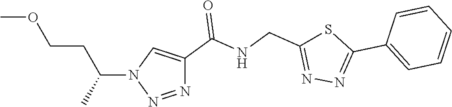 [SMILES: COCC[C@@H](C)n1cc(C(=O)NCc2nnc(-c3ccccc3)s2)nn1] | (R)-1-(4-methoxybutan-2- yl)-N-((5-phenyl-1,3,4- thiadiazol-2-yl)methyl)-1H- 1,2,3-triazole-4- carboxamide | 373 |
| Example 66 | 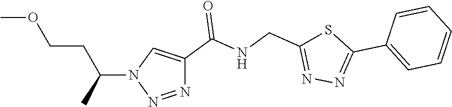 [SMILES: COCC[C@H](C)n1cc(C(=O)NCc2nnc(-c3ccccc3)s2)nn1] | (S)-1-(4-methoxybutan-2- yl)-N-((5-phenyl-1,3,4- thiadiazol-2-yl)methyl)-1H- 1,2,3-triazole-4- carboxamide | 373 |
| Example 67 | 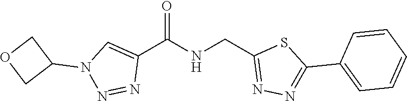 [SMILES: O=C(NCc1nnc(-c2ccccc2)s1)c1cn(C2COC2)nn1] | 1-(oxetan-3-yl)-N-((5- phenyl-1,3,4-thiadiazol-2- yl)methyl)-1H-1,2,3- triazole-4-carboxamide | 343 |
| Example 68 | 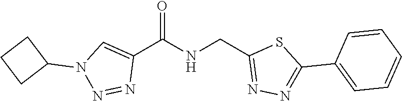 [SMILES: O=C(NCc1nnc(-c2ccccc2)s1)c1cn(C2CCC2)nn1] | 1-cyclobutyl-N-((5-phenyl- 1,3,4-thiadiazol-2- yl)methyl)-1H-1,2,3- triazole-4-carboxamide | 341 |
| Example 69 | 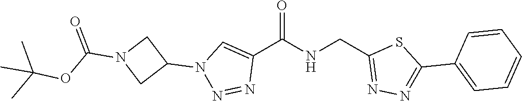 [SMILES: CC(C)(C)OC(=O)N1CC(n2cc(C(=O)NCc3nnc(-c4ccccc4)s3)nn2)C1] | tert-butyl 3-(4-(((5-phenyl- 1,3,4-thiadiazol-2- yl)methyl)carbamoyl)-1H- 1,2,3-triazol-1-yl)azetidine- 1-carboxylate | 442 |
| Example 70 | 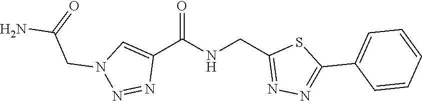 [SMILES: NC(=O)Cn1cc(C(=O)NCc2nnc(-c3ccccc3)s2)nn1] | 1-(2-amino-2-oxoethyl)-N- ((5-phenyl-1,3,4-thiadiazol- 2-yl)methyl)-1H-1,2,3- triazole-4-carboxamide | 344 |
| Example 71 | 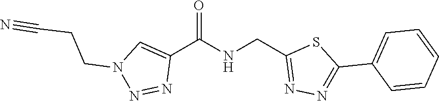 [SMILES: N#CCCn1cc(C(=O)NCc2nnc(-c3ccccc3)s2)nn1] | 1-(2-cyanoethyl)-N-((5- phenyl-1,3,4-thiadiazol-2- yl)methyl)-1H-1,2,3- triazole-4-carboxamide | 340 |
| Example 72 | 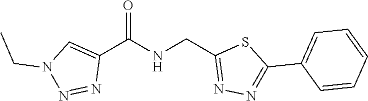 [SMILES: CCn1cc(C(=O)NCc2nnc(-c3ccccc3)s2)nn1] | 1-ethyl-N-((5-phenyl-1,3,4- thiadiazol-2-yl)methyl)-1H- 1,2,3-triazole-4- carboxamide | 315 |
| Example 73 | 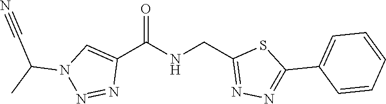 [SMILES: CC(C#N)n1cc(C(=O)NCc2nnc(-c3ccccc3)s2)nn1] | 1-(1-cyanoethyl)-N-((5- phenyl-1,3,4-thiadiazol-2- yl)methyl)-1H-1,2,3- triazole-4-carboxamide | 340 |
| Example 74 | 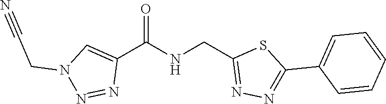 [SMILES: N#CCn1cc(C(=O)NCc2nnc(-c3ccccc3)s2)nn1] | 1-(cyanomethyl)-N-((5- phenyl-1,3,4-thiadiazol-2- yl)methyl)-1H-1,2,3- triazole-4-carboxamide | 326 |
| Example 75 | 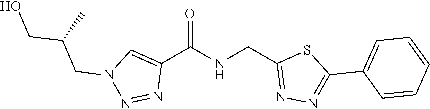 [SMILES: C[C@@H](CO)Cn1cc(C(=O)NCc2nnc(-c3ccccc3)s2)nn1] | (R)-1-(3-hydroxy-2- methylpropyl)-N-((5- phenyl-1,3,4-thiadiazol-2- yl)methyl)-1H-1,2,3- triazole-4-carboxamide | 359 |
| Example 76 | 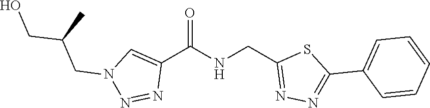 [SMILES: C[C@H](CO)Cn1cc(C(=O)NCc2nnc(-c3ccccc3)s2)nn1] | (S)-1-(3-hydroxy-2- methylpropyl)-N-((5- phenyl-1,3,4-thiadiazol-2- yl)methyl)-1H-1,2,3- triazole-4-carboxamide | 359 |
| Example 77 | 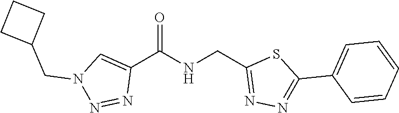 [SMILES: O=C(NCc1nnc(-c2ccccc2)s1)c1cn(CC2CCC2)nn1] | 1-(cyclobutylmethyl)-N-((5- phenyl-1,3,4-thiadiazol-2- yl)methyl)-1H-1,2,3- triazole-4-carboxamide | 355 |
| Example 78 | 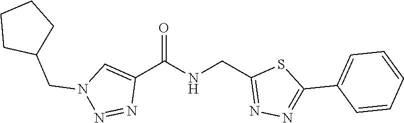 [SMILES: O=C(NCc1nnc(-c2ccccc2)s1)c1cn(CC2CCCC2)nn1] | 1-(cyclopentylmethyl)-N- ((5-phenyl-1,3,4-thiadiazol- 2-yl)methyl)-1H-1,2,3- triazole-4-carboxamide | 369 |
| Example 79 | 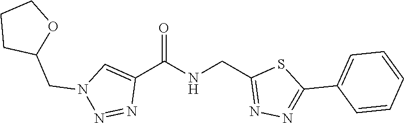 [SMILES: O=C(NCc1nnc(-c2ccccc2)s1)c1cn(CC2CCCO2)nn1] | N-((5-phenyl-1,3,4- thiadiazol-2-yl)methyl)-1- ((tetrahydrofuran-2- yl)methyl)-1H-1,2,3- triazole-4-carboxamide | 371 |
| Example 80 | 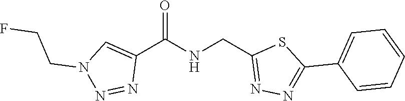 [SMILES: O=C(NCc1nnc(-c2ccccc2)s1)c1cn(CCF)nn1] | 1-(2-fluoroethyl)-N-((5- phenyl-1,3,4-thiadiazol-2- yl)methyl)-1H-1,2,3- triazole-4-carboxamide | 333 |
| Example 81 | 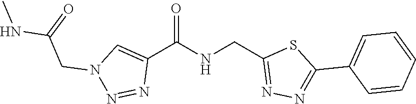 [SMILES: CNC(=O)Cn1cc(C(=O)NCc2nnc(-c3ccccc3)s2)nn1] | 1-(2-(methylamino)-2- oxoethyl)-N-((5-phenyl- 1,3,4-thiadiazol-2- yl)methyl)-1H-1,2,3- triazole-4-carboxamide | 358 |
| Example 82 | 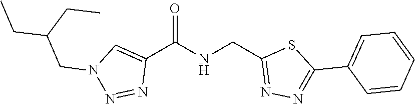 [SMILES: CCC(CC)Cn1cc(C(=O)NCc2nnc(-c3ccccc3)s2)nn1] | 1-(2-ethylbutyl)-N-((5- phenyl-1,3,4-thiadiazol-2- yl)methyl)-1H-1,2,3- triazole-4-carboxamide | 371 |
| Example 83 | 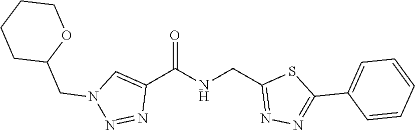 [SMILES: O=C(NCc1nnc(-c2ccccc2)s1)c1cn(CC2CCCCO2)nn1] | N-((5-phenyl-1,3,4- thiadiazol-2-yl)methyl)-1- ((tetrahydro-2H-pyran-2- yl)methyl)-1H-1,2,3- triazole-4-carboxamide | 385 |
| Example 84 | 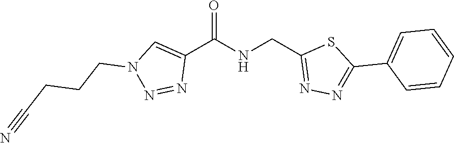 [SMILES: N#CCCCn1cc(C(=O)NCc2nnc(-c3ccccc3)s2)nn1] | 1-(3-cyanopropyl)-N-((5- phenyl-1,3,4-thiadiazol-2- yl)methyl)-1H-1,2,3- triazole-4-carboxamide | 354 |
| Example 85 | 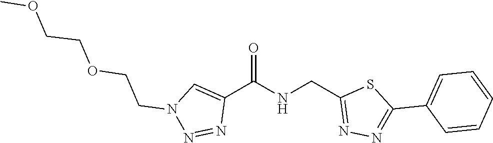 [SMILES: COCCOCCn1cc(C(=O)NCc2nnc(-c3ccccc3)s2)nn1] | 1-(2-(2- methoxyethoxy)ethyl)-N- ((5-phenyl-1,3,4-thiadiazol- 2-yl)methyl)-1H-1,2,3- triazole-4-carboxamide | 389 |
| Example 86 | 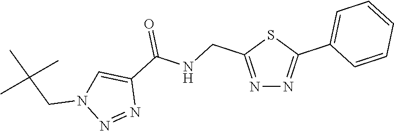 [SMILES: CC(C)(C)Cn1cc(C(=O)NCc2nnc(-c3ccccc3)s2)nn1] | 1-neopentyl-N-((5-phenyl- 1,3,4-thiadiazol-2- yl)methyl)-1H-1,2,3- triazole-4-carboxamide | 357 |
| Example 87 | 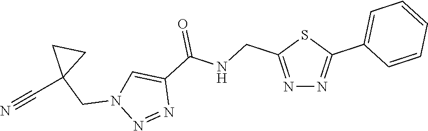 [SMILES: N#CC1(Cn2cc(C(=O)NCc3nnc(-c4ccccc4)s3)nn2)CC1] | 1-((1- cyanocyclopropyl)methyl)- N-((5-phenyl-1,3,4- thiadiazol-2-yl)methyl)-1H- 1,2,3-triazole-4- carboxamide | 367 |
| Example 88 | 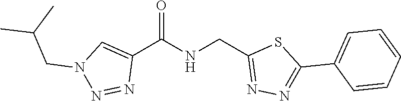 [SMILES: CC(C)Cn1cc(C(=O)NCc2nnc(-c3ccccc3)s2)nn1] | 1-isobutyl-N-((5-phenyl- 1,3,4-thiadiazol-2- yl)methyl)-1H-1,2,3- triazole-4-carboxamide | 343 |
| Example 89 | 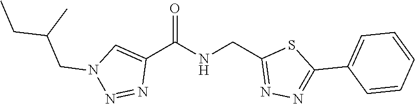 [SMILES: CCC(C)Cn1cc(C(=O)NCc2nnc(-c3ccccc3)s2)nn1] | 1-(2-methylbutyl)-N-((5- phenyl-1,3,4-thiadiazol-2- yl)methyl)-1H-1,2,3- triazole-4-carboxamide | 357 |
| Example 90 | 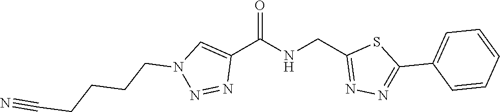 [SMILES: N#CCCCCn1cc(C(=O)NCc2nnc(-c3ccccc3)s2)nn1] | 1-(4-cyanobutyl)-N-((5- phenyl-1,3,4-thiadiazol-2- yl)methyl)-1H-1,2,3- triazole-4-carboxamide | 368 |
| Example 91 | 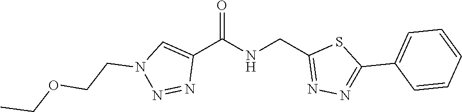 [SMILES: CCOCCn1cc(C(=O)NCc2nnc(-c3ccccc3)s2)nn1] | 1-(2-ethoxyethyl)-N-((5- phenyl-1,3,4-thiadiazol-2- yl)methyl)-1H-1,2,3- triazole-4-carboxamide | 359 |
| Example 92 | 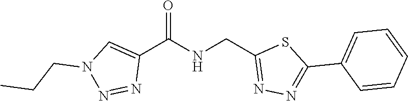 [SMILES: CCCn1cc(C(=O)NCc2nnc(-c3ccccc3)s2)nn1] | N-((5-phenyl-1,3,4- thiadiazol-2-yl)methyl)-1- propyl-1H-1,2,3-triazole-4- carboxamide | 329 |
| Example 93 | 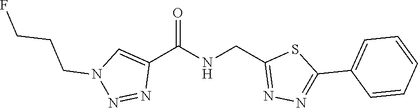 [SMILES: O=C(NCc1nnc(-c2ccccc2)s1)c1cn(CCCF)nn1] | 1-(3-fluoropropyl)-N-((5- phenyl-1,3,4-thiadiazol-2- yl)methyl)-1H-1,2,3- triazole-4-carboxamide | 347 |
| Example 94 | 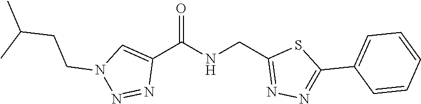 [SMILES: CC(C)CCn1cc(C(=O)NCc2nnc(-c3ccccc3)s2)nn1] | 1-isopentyl-N-((5-phenyl- 1,3,4-thiadiazol-2- yl)methyl)-1H-1,2,3- triazole-4-carboxamide | 357 |
| Example 95 | 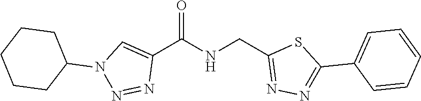 [SMILES: O=C(NCc1nnc(-c2ccccc2)s1)c1cn(C2CCCCC2)nn1] | 1-cyclohexyl-N-((5-phenyl- 1,3,4-thiadiazol-2- yl)methyl)-1H-1,2,3- triazole-4-carboxamide | 369 |
| Example 96 | 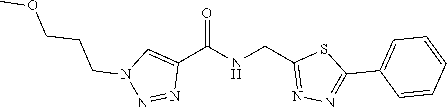 [SMILES: COCCCn1cc(C(=O)NCc2nnc(-c3ccccc3)s2)nn1] | 1-(3-methoxypropyl)-N-((5- phenyl-1,3,4-thiadiazol-2- yl)methyl)-1H-1,2,3- triazole-4-carboxamide | 359 |
| Example 97 | 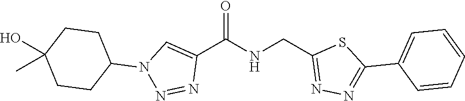 [SMILES: CC1(O)CCC(n2cc(C(=O)NCc3nnc(-c4ccccc4)s3)nn2)CC1] | 1-(4-hydroxy-4- methylcyclohexyl)-N-((5- phenyl-1,3,4-thiadiazol-2- yl)methyl)-1H-1,2,3- triazole-4-carboxamide | 399 |
| Example 98 | 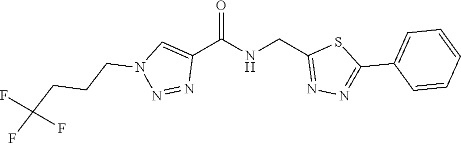 [SMILES: O=C(NCc1nnc(-c2ccccc2)s1)c1cn(CCCC(F)(F)F)nn1] | N-((5-phenyl-1,3,4- thiadiazol-2-yl)methyl)-1- (4,4,4-trifluorobutyl)-1H- 1,2,3-triazole-4- carboxamide | 397 |
| Example 99 | 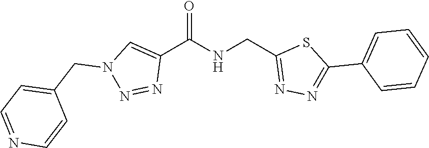 [SMILES: O=C(NCc1nnc(-c2ccccc2)s1)c1cn(Cc2ccncc2)nn1] | N-((5-phenyl-1,3,4- thiadiazol-2-yl)methyl)-1- (pyridin-4-ylmethyl)-1H- 1,2,3-triazole-4- carboxamide | 378 |
| Example 100 | 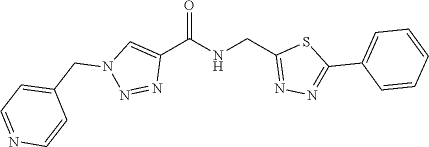 [SMILES: O=C(NCc1nnc(-c2ccccc2)s1)c1cn(Cc2ccncc2)nn1] | N-((5-phenyl-1,3,4- thiadiazol-2-yl)methyl)-1- (pyridin-3-ylmethyl)-1H- 1,2,3-triazole-4- carboxamide | 378 |
| Example 101 | 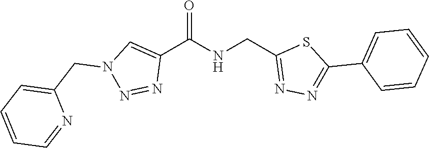 [SMILES: O=C(NCc1nnc(-c2ccccc2)s1)c1cn(Cc2ccccn2)nn1] | N-((5-phenyl-1,3,4- thiadiazol-2-yl)methyl)-1- (pyridin-2-ylmethyl)-1H- 1,2,3-triazole-4- carboxamide | 378 |

### Separation of Racemic N-((5-phenyl-1,3,4-thiadiazol-2-yl)methyl)-1-(4,4,4-trifluorobutan-2-yl)-1H-1,2,3-triazole-4-carboxamide, Example 63 and 64

The racemic mixture of N-((5-phenyl-1,3,4-thiadiazol-2-yl)methyl)-1-(4,4,4-trifluorobutan-2-yl)-1H-1,2,3-triazole-4-carboxamide was resolved by chiral SFC purification (IZ, 21×250 mm column, 25% MeOH w/0.1% NH₄OH as cosolvent).

### Example 102

### Preparation of 1-(1-acetylazetidin-3-yl)-N-((5-phenyl-1,3,4-thiadiazol-2-yl)methyl)-1H-1,2,3-triazole-4-carboxamide

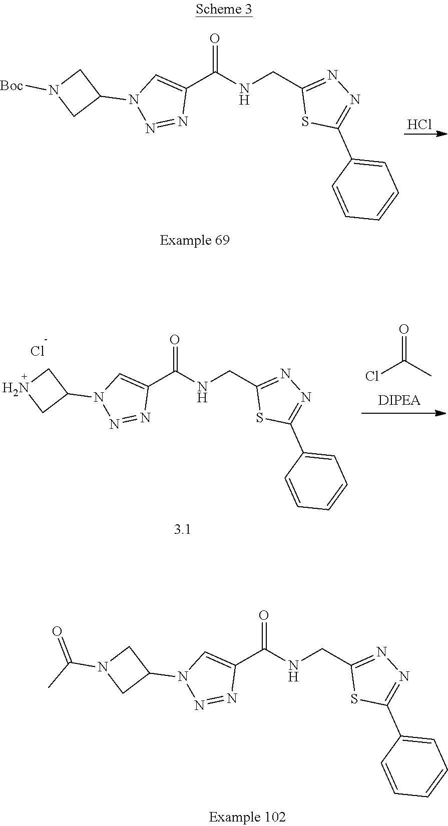

### Step 1. Preparation of 3-(4-(((5-phenyl-1,3,4-thiadiazol-2-yl)methyl)carbamoyl)-1H-1,2,3-triazol-1-yl)azetidin-1-ium chloride

HCl in dioxane (5 mL, 20 mmol, 4 M) was added to the stirred solution of tert-butyl 3-(4-(((5-phenyl-1,3,4-thiadiazol-2-yl)methyl)carbamoyl)-1H-1,2,3-triazol-1-yl)azetidine-1-carboxylate Example 69 (1.7 g, 3.7 mmol) in DCM (15 mL) under N₂ atmosphere at 0° C. dropwise. The resulting reaction mixture was stirred to 25° C. for 4 hours. The reaction mixture was concentrated under reduced pressure and the residue was triturated with diethyl ether to afford 3-(4-(((5-phenyl-1,3,4-thiadiazol-2-yl)methyl)carbamoyl)-1H-1,2,3-triazol-1-yl)azetidin-1-ium chloride. LC/MS (m/z): 342 (M+H)⁺.

### Step 2. Preparation of 1-(1-acetylazetidin-3-yl)-N-((5-phenyl-1,3,4-thiadiazol-2-yl)methyl)-1H-1,2,3-triazole-4-carboxamide

DIPEA (0.135 mL, 0.770 mmol), and acetyl chloride (0.024 mL, 0.334 mmol) was added to a stirred solution of 3-(4-(((5-phenyl-1,3,4-thiadiazol-2-yl)methyl)carbamoyl)-1H-1,2,3-triazol-1-yl)azetidin-1-ium chloride (100 mg, 0.257 mmol) in dioxane (3 mL) at 0° C. and the reaction mixture was stirred at 25° C. for 16 hours. The reaction mixture was diluted with water (30 mL) and extracted with EtOAc (3×20 mL). The organic layer was dried over Na₂SO₄, filtered and concentrated under reduced pressure. The residue was purified via reverse phase HPLC (eluting MeCN in water with ammonium bicarbonate modifier) to afford 1-(1-acetylazetidin-3-yl)-N-((5-phenyl-1,3,4-thiadiazol-2-yl)methyl)-1H-1,2,3-triazole-4-carboxamide. LC/MS (m/z): 484 (M+H)⁺.

Examples shown in Example Table 3 below, were prepared according to procedures analogous to those outlined in Example 102 using the appropriate starting materials obtained from methods described above or elsewhere, or as obtained from commercial sources.

EXAMPLE TABLE 3

| Example | Structure | Name | Mass [M + H]⁺ |
|---|---|---|---|
| Example 103 | 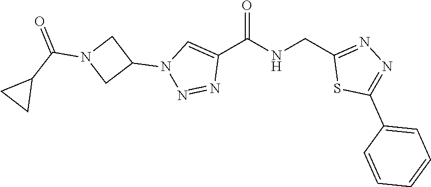 [SMILES: O=C(NCc1nnc(-c2ccccc2)s1)c1cn(C2CN(C(=O)C3CC3)C2)nn1] | 1-(1- (cyclopropanecarbonyl) azetidin-3-yl)-N-((5-phenyl- 1,3,4-thiadiazol-2- yl)methyl)-1H-1,2,3- triazole-4-carboxamide | 410 |

### Example 104

### Preparation of N-((5-(3-chloro-2-fluorophenyl)-1,3,4-thiadiazol-2-yl)methyl)-1-(2,6-dimethylpyridin-3-yl)-1H-1,2,3-triazole-4-carboxamide

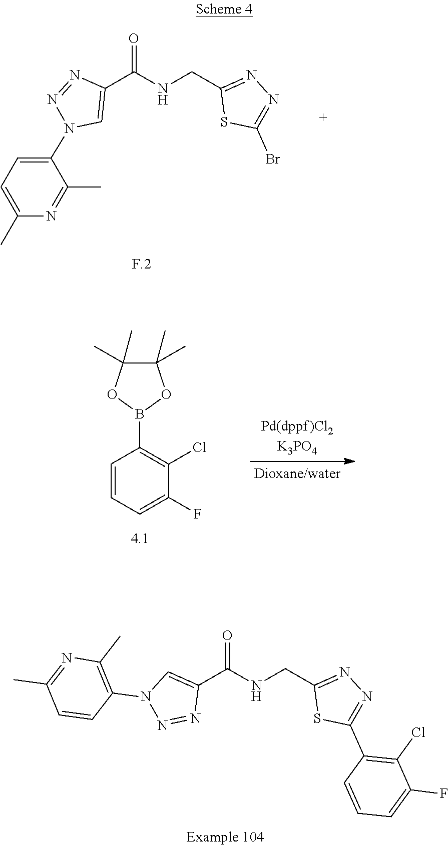 [SMILES: Cc1ccc(-n2cc(C(=O)NCc3nnc(Br)s3)nn2)c(C)n1 ; CC1(C)OB(c2cccc(F)c2Cl)OC1(C)C ; Cc1ccc(-n2cc(C(=O)NCc3nnc(-c4cccc(F)c4Cl)s3)nn2)c(C)n1]

An 8 mL vial was charged with N-((5-bromo-1,3,4-thiadiazol-2-yl)methyl)-1-(2,6-dimethylpyridin-3-yl)-1H-1,2,3-triazole-4-carboxamide (15.8 mg, 40.0 μmol), 2-(2-chloro-3-fluorophenyl)-4,4,5,5-tetramethyl-1,3,2-dioxaborolane (21 mg, 80.0 μmol), potassium phosphate tribasic (25.4 mg, 120 μmol) and [1,1′-bis(diphenylphosphino)ferrocene]dichloropalladium(II) (5.85 mg, 8.00 μmol eq) under an inert atmosphere. Dioxane (400 μL) and H₂O (40.0 μL) were added and the reaction was heated to 120° C. for 0.5 hours in a microwave reactor. The reaction mixture was cooled to room temperature, concentrated by speedvac, and the residue was purified via reverse phase HPLC (eluting acetonitrile in water, with TFA modifier) to afford N-((5-(3-chloro-2-fluorophenyl)-1,3,4-thiadiazol-2-yl)methyl)-1-(2,6-dimethylpyridin-3-yl)-1H-1,2,3-triazole-4-carboxamide. LC/MS (m/z): 444 (M+H)⁺.

Examples shown in Example Table 4 below, were or may be prepared according to procedures analogous to those outlined in Example 104 and General Scheme 4 above using the appropriate starting materials obtained from methods described above or elsewhere, or as obtained from commercial sources.

EXAMPLE TABLE 4

| Example | Structure | Name | Mass [M + H]⁺ |
|---|---|---|---|
| Example 105 | 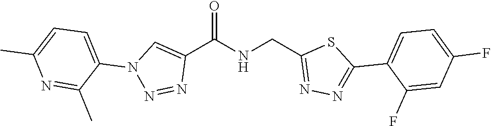 [SMILES: Cc1ccc(-n2cc(C(=O)NCc3nnc(-c4ccc(F)cc4F)s3)nn2)c(C)n1] | N-((5-(2,4-difluorophenyl)- 1,3,4-thiadiazol-2- yl)methyl)-1-(2,6- dimethylpyridin-3-yl)-1H- 1,2,3-triazole-4- carboxamide | 428 |
| Example 106 | 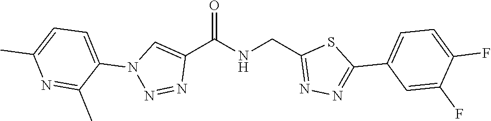 [SMILES: Cc1ccc(-n2cc(C(=O)NCc3nnc(-c4ccc(F)c(F)c4)s3)nn2)c(C)n1] | N-((5-(3,4-difluorophenyl)- 1,3,4-thiadiazol-2- yl)methyl)-1-(2,6- dimethylpyridin-3-yl)-1H- 1,2,3-triazole-4- carboxamide | 428 |
| Example 107 | 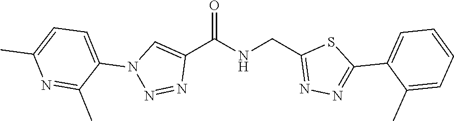 [SMILES: Cc1ccc(-n2cc(C(=O)NCc3nnc(-c4ccccc4C)s3)nn2)c(C)n1] | 1-(2,6-dimethylpyridin-3- yl)-N-((5-(o-tolyl)-1,3,4- thiadiazol-2-yl)methyl)-1H- 1,2,3-triazole-4- carboxamide | 406 |
| Example 108 | 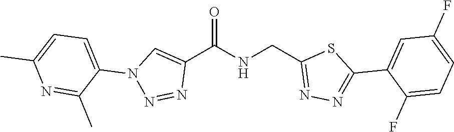 [SMILES: Cc1ccc(-n2cc(C(=O)NCc3nnc(-c4cc(F)ccc4F)s3)nn2)c(C)n1] | N-((5-(2,5-difluorophenyl)- 1,3,4-thiadiazol-2- yl)methyl)-1-(2,6- dimethylpyridin-3-yl)-1H- 1,2,3-triazole-4- carboxamide | 428 |
| Example 109 | 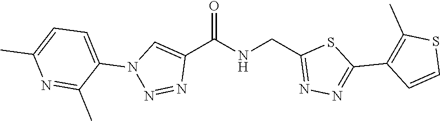 [SMILES: Cc1ccc(-n2cc(C(=O)NCc3nnc(-c4ccsc4C)s3)nn2)c(C)n1] | 1-(2,6-dimethylpyridin-3- yl)-N-((5-(2- methylthiophen-3-yl)-1,3,4- thiadiazol-2-yl)methyl)-1H- 1,2,3-triazole-4- carboxamide | 412 |
| Example 110 | 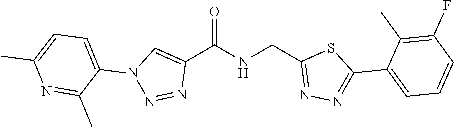 [SMILES: Cc1ccc(-n2cc(C(=O)NCc3nnc(-c4cccc(F)c4C)s3)nn2)c(C)n1] | 1-(2,6-dimethylpyridin-3- yl)-N-((5-(3-fluoro-2- methylphenyl)-1,3,4- thiadiazol-2-yl)methyl)-1H- 1,2,3-triazole-4- carboxamide | 424 |
| Example 111 | 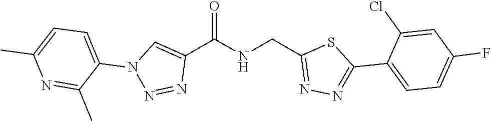 [SMILES: Cc1ccc(-n2cc(C(=O)NCc3nnc(-c4ccc(F)cc4Cl)s3)nn2)c(C)n1] | N-((5-(2-chloro-4- fluorophenyl)-1,3,4- thiadiazol-2-yl)methyl)-1- (2,6-dimethylpyridin-3-yl)- 1H-1,2,3-triazole-4- carboxamide | 444 |
| Example 112 | 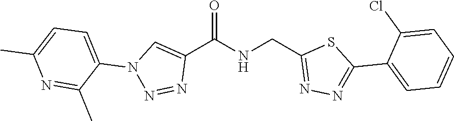 [SMILES: Cc1ccc(-n2cc(C(=O)NCc3nnc(-c4ccccc4Cl)s3)nn2)c(C)n1] | N-((5-(2-chlorophenyl)- 1,3,4-thiadiazol-2- yl)methyl)-1-(2,6- dimethylpyridin-3-yl)-1H- 1,2,3-triazole-4- carboxamide | 426 |
| Example 113 | 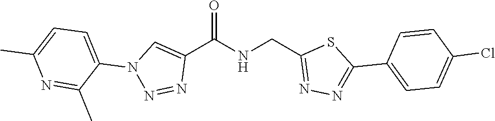 [SMILES: Cc1ccc(-n2cc(C(=O)NCc3nnc(-c4ccc(Cl)cc4)s3)nn2)c(C)n1] | N-((5-(4-chlorophenyl)- 1,3,4-thiadiazol-2- yl)methyl)-1-(2,6- dimethylpyridin-3-yl)-1H- 1,2,3-triazole-4- carboxamide | 426 |
| Example 114 | 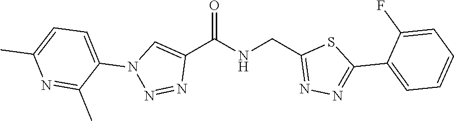 [SMILES: Cc1ccc(-n2cc(C(=O)NCc3nnc(-c4ccccc4F)s3)nn2)c(C)n1] | 1-(2,6-dimethylpyridin-3- yl)-N-((5-(2-fluorophenyl)- 1,3,4-thiadiazol-2- yl)methyl)-1H-1,2,3- triazole-4-carboxamide | 410 |
| Example 115 | 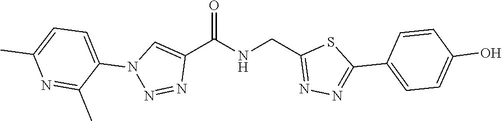 [SMILES: Cc1ccc(-n2cc(C(=O)NCc3nnc(-c4ccc(O)cc4)s3)nn2)c(C)n1] | 1-(2,6-dimethylpyridin-3- yl)-N-((5-(4- hydroxyphenyl)-1,3,4- thiadiazol-2-yl)methyl)-1H- 1,2,3-triazole-4- carboxamide | 408 |

### Example 116

### Preparation of 1-(2,6-dimethylpyridin-3-yl)-N-((6-(2-fluorophenyl)pyridazin-3-yl)methyl)-1H-1,2,3-triazole-4-carboxamide

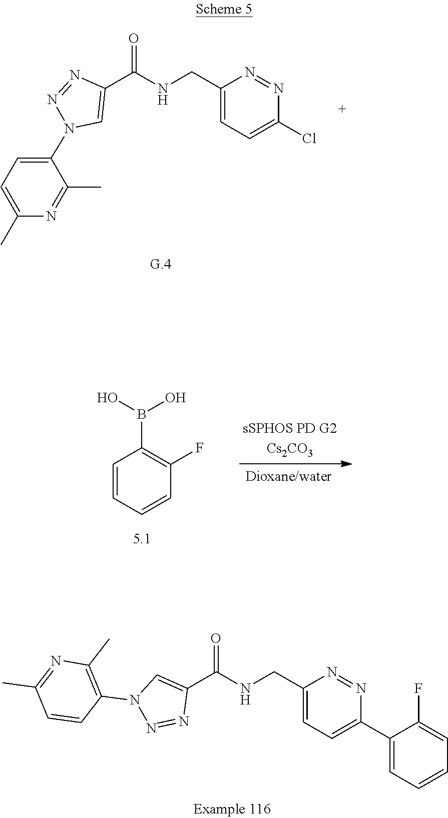 [SMILES: Cc1ccc(-n2cc(C(=O)NCc3ccc(Cl)nn3)nn2)c(C)n1 ; OB(O)c1ccccc1F ; Cc1ccc(-n2cc(C(=O)NCc3ccc(-c4ccccc4F)nn3)nn2)c(C)n1]

An 8 mL vials was charged with N-((6-chloropyridazin-3-yl)methyl)-1-(2,6-dimethylpyridin-3-yl)-1H-1,2,3-triazole-4-carboxamide (20.0 mg, 60.0 μmol), (2-fluorophenyl)boronic acid (13 mg, 90.0 μmol), Chloro(sodium-2-dicyclohexylphosphino-2′,6′-dimethoxy-1,1′-biphenyl-3′-sulfonate)[2-(2′-amino-1,1′-biphenyl)]palladium(II) (9.87 mg, 12.0 μmol) and Cesium carbonate (58.5 mg, 180 μmol) under and inert atmosphere. Dioxane (600 μL) and H₂O (200 μL) were added and reaction was heated to 100° C. for 16 hours. The reaction mixture was cooled to room temperature, concentrated by speedvac, and the residue was purified via reverse phase HPLC (eluting acetonitrile in water, with TFA modifier) to afford 1-(2,6-dimethylpyridin-3-yl)-N-((6-(2-fluorophenyl)pyridazin-3-yl)methyl)-1H-1,2,3-triazole-4-carboxamide. LC/MS (m/z): 404 (M+H)⁺.

Examples shown in Example Table 5 below, were prepared according to procedures analogous to those outlined in Example 116 and General Scheme 4 above using the appropriate staffing materials obtained from methods described above or elsewhere, or as obtained from commercial sources.

EXAMPLE TABLE 5

| Example | Structure | Name | Mass [M + H]⁺ |
|---|---|---|---|
| Example 117 | 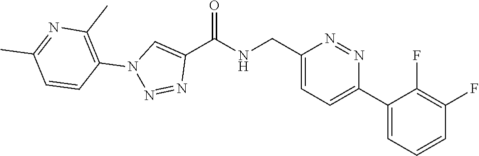 [SMILES: Cc1ccc(-n2cc(C(=O)NCc3ccc(-c4cccc(F)c4F)nn3)nn2)c(C)n1] | N-((6-(2,3- difluorophenyl)pyridazin-3- yl)methyl)-1-(2,6- dimethylpyridin-3-yl)-1H- 1,2,3-triazole-4- carboxamide | 422 |
| Example 118 | 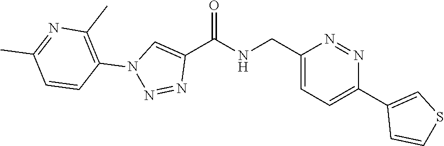 [SMILES: Cc1ccc(-n2cc(C(=O)NCc3ccc(-c4ccsc4)nn3)nn2)c(C)n1] | 1-(2,6-dimethylpyridin-3- yl)-N-((6-(thiophen-3- yl)pyridazin-3-yl)methyl)- 1H-1,2,3-triazole-4- carboxamide | 392 |
| Example 119 | 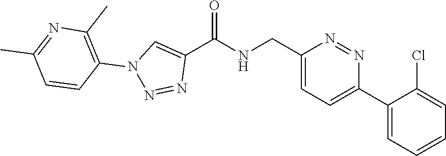 [SMILES: Cc1ccc(-n2cc(C(=O)NCc3ccc(-c4ccccc4Cl)nn3)nn2)c(C)n1] | N-((6-(2- chlorophenyl)pyridazin-3- yl)methyl)-1-(2,6- dimethylpyridin-3-yl)-1H- 1,2,3-triazole-4- carboxamide | 420 |
| Example 120 | 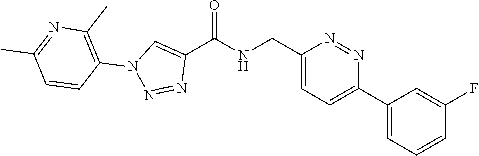 [SMILES: Cc1ccc(-n2cc(C(=O)NCc3ccc(-c4cccc(F)c4)nn3)nn2)c(C)n1] | 1-(2,6-dimethylpyridin-3- yl)-N-((6-(3- fluorophenyl)pyridazin-3- yl)methyl)-1H-1,2,3- triazole-4-carboxamide | 404 |
| Example 121 | 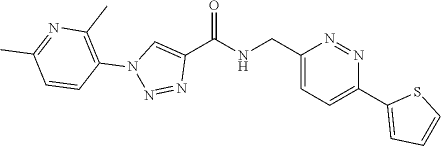 [SMILES: Cc1ccc(-n2cc(C(=O)NCc3ccc(-c4cccs4)nn3)nn2)c(C)n1] | 1-(2,6-dimethylpyridin-3- yl)-N-((6-(thiophen-2- yl)pyridazin-3-yl)methyl)- 1H-1,2,3-triazole-4- carboxamide | 392 |
| Example 122 | 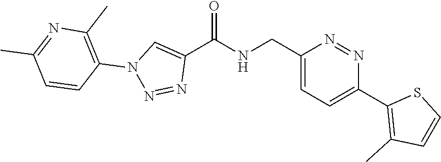 [SMILES: Cc1ccc(-n2cc(C(=O)NCc3ccc(-c4sccc4C)nn3)nn2)c(C)n1] | 1-(2,6-dimethylpyridin-3- yl)-N-((6-(3- methylthiophen-2- yl)pyridazin-3-yl)methyl)- 1H-1,2,3-triazole-4- carboxamide | 406 |
| Example 123 | 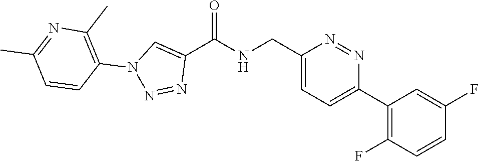 [SMILES: Cc1ccc(-n2cc(C(=O)NCc3ccc(-c4cc(F)ccc4F)nn3)nn2)c(C)n1] | N-((6-(2,5- difluorophenyl)pyridazin-3- yl)methyl)-1-(2,6- dimethylpyridin-3-yl)-1H- 1,2,3-triazole-4- carboxamide | 422 |

### Example 124

### Preparation of 1-(1-methylcyclobutyl)-N-((5-phenyl-1,3,4-thiadiazol-2-yl)methyl)-1H-1,2,3-triazole-4-carboxamide

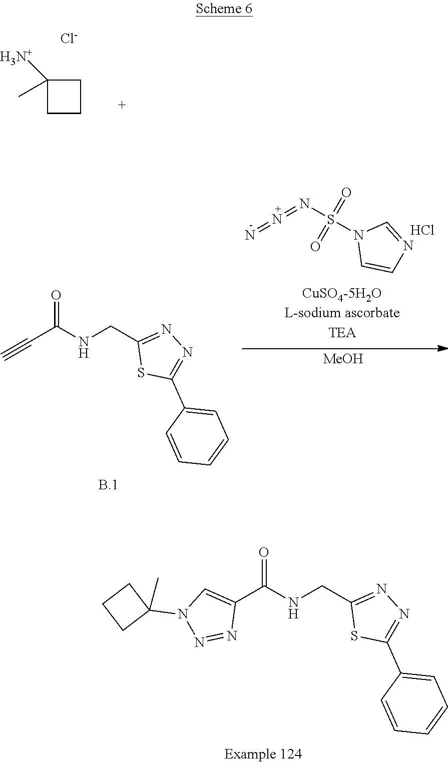

### Preparation of 1-(1-methylcyclobutyl)-N-((5-phenyl-1,3,4-thiadiazol-2-yl)methyl)-1H-1,2,3-triazole-4-carboxamide

N-((5-phenyl-1,3,4-thiadiazol-2-yl)methyl)propiolamide (48.7 mg, 0.200 mmol), 1-methylcyclobutan-1-aminium chloride (24 mg, 0.200 mmol), copper(II) sulfate pentahydrate (4.99 mg, 0.020 mmol), L-ascorbic acid sodium salt (7.92 mg, 0.040 mmol) and 1-(azidosulfonyl)-1H-imidazol-3-ium chloride (41.9 mg, 0.200 mmol) were charged in a 2 dram vial with stir bar and septum and the vial was evacuated and backfilled with argon (3×), MeOH (1000 μL) was added followed by triethylamine (55.8 μL, 0.400 mmol) and the reaction was stirred at room temperature. The reaction was diluted with DMSO (1 mL), filtered and purified reverse phase HPLC (eluting acetonitrile in water, with TFA modifier) to afford 1-(1-methylcyclobutyl)-N-((5-phenyl-1,3,4-thiadiazol-2-yl)methyl)-1H-1,2,3-triazole-4-carboxamide. LC/MS (m/z): 355 (M+H)⁺.

Examples shown in Example Table 6 below, were prepared according to procedures analogous to those outlined in Example 124 and General Scheme 3 above using the appropriate starting materials obtained from methods described above or elsewhere, or as obtained from commercial sources.

EXAMPLE TABLE 6

| Example | Structure | Name | Mass [M + H]⁺ |
|---|---|---|---|
| Example 125 | 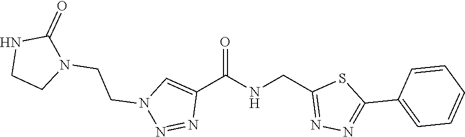 [SMILES: O=C(NCc1nnc(-c2ccccc2)s1)c1cn(CCN2CCNC2=O)nn1] | 1-(2-(2-oxoimidazolidin-1- yl)ethyl)-N-((5-phenyl- 1,3,4-thiadiazol-2- yl)methyl)-1H-1,2,3- triazole-4-carboxamide | 399 |
| Example 126 | 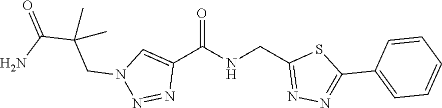 [SMILES: CC(C)(Cn1cc(C(=O)NCc2nnc(-c3ccccc3)s2)nn1)C(N)=O] | 1-(3-amino-2,2-dimethyl-3- oxo-propyl)-N-[(5-phenyl- 1,3,4-thiadiazol-2- yl)methyl]triazole-4- carboxamide | 386 |
| Example 127 | 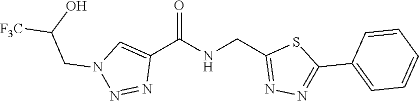 [SMILES: O=C(NCc1nnc(-c2ccccc2)s1)c1cn(CC(O)C(F)(F)F)nn1] | N-((5-phenyl-1,3,4- thiadiazol-2-yl)methyl)-1- (3,3,3-trifluoro-2- hydroxypropyl)-1H-1,2,3- triazole-4-carboxamide | 399 |
| Example 128 | 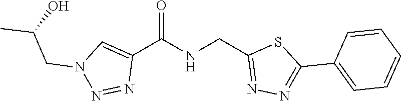 [SMILES: C[C@H](O)Cn1cc(C(=O)NCc2nnc(-c3ccccc3)s2)nn1] | (S)-1-(2-hydroxypropyl)-N- ((5-phenyl-1,3,4-thiadiazol- 2-yl)methyl)-1H-1,2,3- triazole-4-carboxamide | 345 |
| Example 129 | 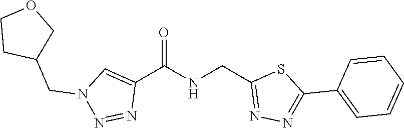 [SMILES: O=C(NCc1nnc(-c2ccccc2)s1)c1cn(CC2CCOC2)nn1] | N-((5-phenyl-1,3,4- thiadiazol-2-yl)methyl)-1- ((tetrahydrofuran-3- yl)methyl)-1H-1,2,3- triazole-4-carboxamide | 371 |
| Example 130 | 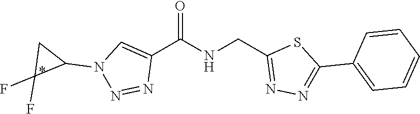 [SMILES: O=C(NCc1nnc(-c2ccccc2)s1)c1cn(C2CC2(F)F)nn1] | 1-(2,2- difluorocyclopropyl)-N-((5- phenyl-1,3,4-thiadiazol-2- yl)methyl)-1H-1,2,3- triazole-4-carboxamide, Peak 2 | 363 |
| Example 131 | 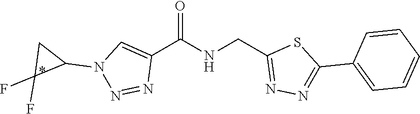 [SMILES: O=C(NCc1nnc(-c2ccccc2)s1)c1cn(C2CC2(F)F)nn1] | 1-(2,2- difluorocyclopropyl)-N-((5- phenyl-1,3,4-thiadiazol-2- yl)methyl)-1H-1,2,3- triazole-4-carboxamide, Peak 1 | 363 |
| Example 132 | 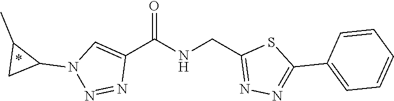 [SMILES: CC1CC1n1cc(C(=O)NCc2nnc(-c3ccccc3)s2)nn1] | Cis-1-(2- methylcyclopropyl)-N-((5- phenyl-1,3,4-thiadiazol-2- yl)methyl)-1H-1,2,3- triazole-4-carboxamide, peak 2 | 341 |
| Example 133 | 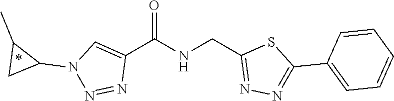 [SMILES: CC1CC1n1cc(C(=O)NCc2nnc(-c3ccccc3)s2)nn1] | Cis-1-(2- methylcyclopropyl)-N-((5- phenyl-1,3,4-thiadiazol-2- yl)methyl)-1H-1,2,3- triazole-4-carboxamide, peak 1 | 341 |
| Example 134 | 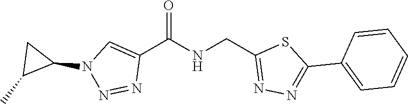 [SMILES: C[C@@H]1C[C@H]1n1cc(C(=O)NCc2nnc(-c3ccccc3)s2)nn1] | 1-((1R,2R)-2- methylcyclopropyl)-N-((5- phenyl-1,3,4-thiadiazol-2- yl)methyl)-1H-1,2,3- triazole-4-carboxamide | 341 |
| Example 135 | 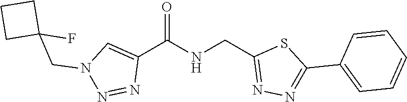 [SMILES: O=C(NCc1nnc(-c2ccccc2)s1)c1cn(CC2(F)CCC2)nn1] | 1-((1- fluorocyclobutyl)methyl)- N-((5-phenyl-1,3,4- thiadiazol-2-yl)methyl)-1H- 1,2,3-triazole-4- carboxamide | 373 |
| Example 136 | 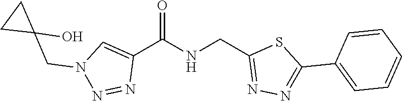 [SMILES: O=C(NCc1nnc(-c2ccccc2)s1)c1cn(CC2(O)CC2)nn1] | 1-((1- hydroxycyclopropyl)methyl)- N-((5-phenyl-1,3,4- thiadiazol-2-yl)methyl)-1H- 1,2,3-triazole-4- carboxamide | 357 |
| Example 137 | 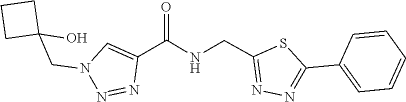 [SMILES: O=C(NCc1nnc(-c2ccccc2)s1)c1cn(CC2(O)CCC2)nn1] | 1-((1- hydroxycyclobutyl)methyl)- N-((5-phenyl-1,3,4- thiadiazol-2-yl)methyl)-1H- 1,2,3-triazole-4- carboxamide | 371 |
| Example 138 | 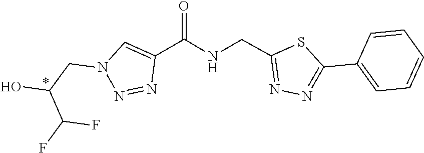 [SMILES: O=C(NCc1nnc(-c2ccccc2)s1)c1cn(CC(O)C(F)F)nn1] | 1-(3,3-difluoro-2- hydroxypropyl)-N-((5- phenyl-1,3,4-thiadiazol-2- yl)methyl)-1H-1,2,3- triazole-4-carboxamide, peak 1 | 381 |
| Example 139 | 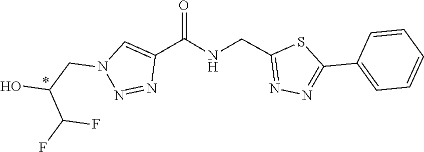 [SMILES: O=C(NCc1nnc(-c2ccccc2)s1)c1cn(CC(O)C(F)F)nn1] | 1-(3,3-difluoro-2- hydroxypropyl)-N-((5- phenyl-1,3,4-thiadiazol-2- yl)methyl)-1H-1,2,3- triazole-4-carboxamide, peak 2 | 381 |
| Example 140 | 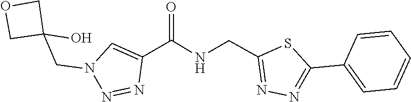 [SMILES: O=C(NCc1nnc(-c2ccccc2)s1)c1cn(CC2(O)COC2)nn1] | 1-((3-hydroxyoxetan-3- yl)methyl)-N-((5-phenyl- 1,3,4-thiadiazol-2- yl)methyl)-1H-1,2,3- triazole-4-carboxamide | 373 |
| Example 141 | 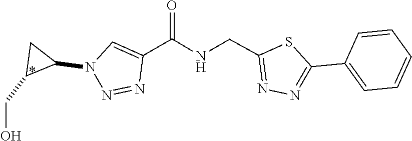 | Trans-1-(2- (hydroxymethyl)cyclopropyl)- N-((5-phenyl-1,3,4- thiadiazol-2-yl)methyl)-1H- 1,2,3-triazole-4- carboxamide, peak 2 | 357 |
| Example 142 | 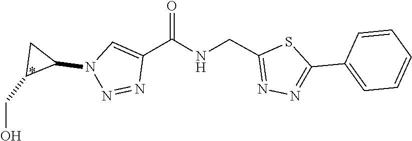 | Trans-1-(2- (hydroxymethyl)cyclopropyl)- N-((5-phenyl-1,3,4- thiadiazol-2-yl)methyl)-1H- 1,2,3-triazole-4- carboxamide, peak 1 | 357 |
| Example 143 | 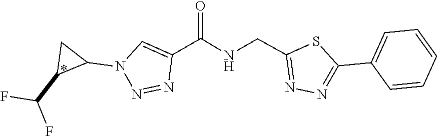 [SMILES: O=C(NCc1nnc(-c2ccccc2)s1)c1cn(C2C[C@@H]2C(F)F)nn1] | Trans-1-(-2- (difluoromethyl)cyclopropyl]- N-[(5-phenyl-1,3,4- thiadiazol-2-yl)methyl]-1H- 1,2,3-triazole-4- carboxamide, peak 1 | 377 |
| Example 144 | 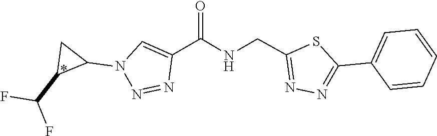 [SMILES: O=C(NCc1nnc(-c2ccccc2)s1)c1cn(C2C[C@@H]2C(F)F)nn1] | Trans-1-(-2- (difluoromethyl)cyclopropyl]- N-[(5-phenyl-1,3,4- thiadiazol-2-yl)methyl]-1H- 1,2,3-triazole-4- carboxamide, peak 2 | 377 |
| Example 145 | 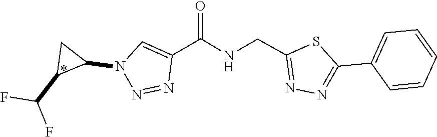 | cis-1-(-2- (difluoromethyl)cyclopropyl]- N-[(5-phenyl-1,3,4- thiadiazol-2-yl)methyl]-1H- 1,2,3-triazole-4- carboxamide, peak 1 | 377 |
| Example 146 | 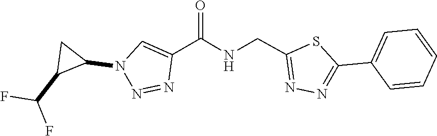 | cis-1-(-2- (difluoromethyl)cyclopropyl]- N-[(5-phenyl-1,3,4- thiadiazol-2-yl)methyl]-1H- 1,2,3-triazole-4- carboxamide, peak 2 | 377 |
| Example 147 | 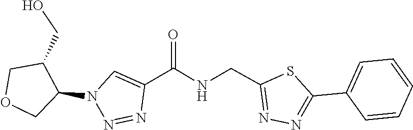 [SMILES: O=C(NCc1nnc(-c2ccccc2)s1)c1cn([C@H]2COC[C@@H]2CO)nn1] | 1-[(3S,4R)-4- (hydroxymethyl)oxolan-3- yl]-N-[(5-phenyl-1,3,4- thiadiazol-2-yl)methyl]-1H- 1,2,3-triazole-4- carboxamide | 387 |
| Example 148 | 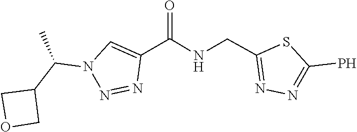 | (S)-1-(1-(oxetan-3- yl)ethyl)-N-((5-phenyl- 1,3,4-thiadiazol-2- yl)methyl)-1H-1,2,3- triazole-4-carboxamide | 371 |
| Example 149 | 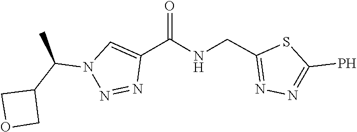 | (R)-1-(1-(oxetan-3- yl)ethyl)-N-((5-phenyl- 1,3,4-thiadiazol-2- yl)methyl)-1H-1,2,3- triazole-4-carboxamide | 371 |
| Example 150 | 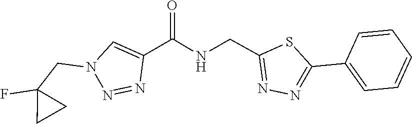 [SMILES: O=C(NCc1nnc(-c2ccccc2)s1)c1cn(CC2(F)CC2)nn1] | 1-((1-fluorocyclopropyl) methyl)-N-((5-phenyl- 1,3,4-thiadiazol-2- yl)methyl)-1H-1,2,3- triazole-4-carboxamide | 359 |
| Example 151 | 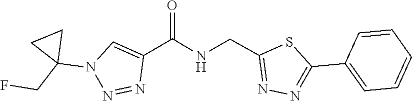 [SMILES: O=C(NCc1nnc(-c2ccccc2)s1)c1cn(C2(CF)CC2)nn1] | 1-(1- (fluoromethyl)cyclopropyl)- N-((5-phenyl-1,3,4- thiadiazol-2-yl)methyl)-1H- 1,2,3-triazole-4- carboxamide | 359 |
| Example 152 | 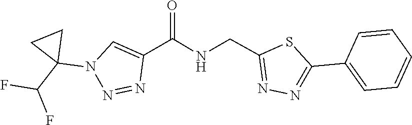 [SMILES: O=C(NCc1nnc(-c2ccccc2)s1)c1cn(C2(C(F)F)CC2)nn1] | 1-(1- (difluoromethyl)cyclopropyl)- N-((5-phenyl-1,3,4- thiadiazol-2-yl)methyl)-1H- 1,2,3-triazole-4- carboxamide | 377 |
| Example 153 | 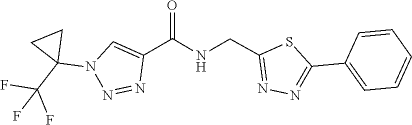 [SMILES: O=C(NCc1nnc(-c2ccccc2)s1)c1cn(C2(C(F)(F)F)CC2)nn1] | N-((5-phenyl-1,3,4- thiadiazol-2-yl)methyl)-1- (1- (trifluoromethyl)cyclopropyl)- 1H-1,2,3-triazole-4- carboxamide | 395 |
| Example 154 | 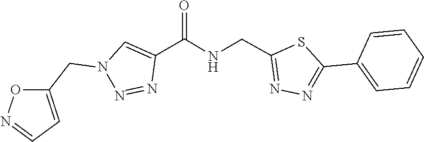 [SMILES: O=C(NCc1nnc(-c2ccccc2)s1)c1cn(Cc2ccno2)nn1] | 1-(isoxazol-5-ylmethyl)-N- ((5-phenyl-1,3,4-thiadiazol- 2-yl)methyl)-1H-1,2,3- triazole-4-carboxamide | 368 |
| Example 155 | 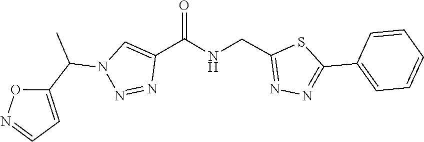 [SMILES: CC(c1ccno1)n1cc(C(=O)NCc2nnc(-c3ccccc3)s2)nn1] | 1-(1-(isoxazol-5-yl) ethyl)-N-((5-phenyl-1,3,4- thiadiazol-2-yl)methyl)-1H- 1,2,3-triazole-4- carboxamide | 382 |
| Example 156 | 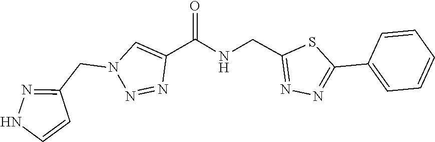 [SMILES: O=C(NCc1nnc(-c2ccccc2)s1)c1cn(Cc2cc[nH]n2)nn1] | 1-((1H-pyrazol-3-yl) methyl)-N-((5-phenyl-1,3,4- thiadiazol-2-yl)methyl)-1H- 1,2,3-triazole-4- carboxamide | 367 |
| Example 157 | 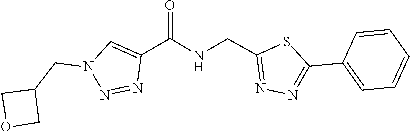 [SMILES: O=C(NCc1nnc(-c2ccccc2)s1)c1cn(CC2COC2)nn1] | 1-(oxetan-3-ylmethyl)-N- ((5-phenyl-1,3,4- thiadiazol-2-yl)methyl)- 1H-1,2,3-triazole-4- carboxamide | 357 |
| Example 158 | 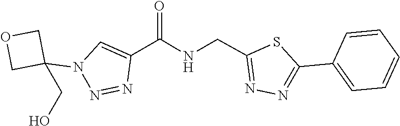 [SMILES: O=C(NCc1nnc(-c2ccccc2)s1)c1cn(C2(CO)COC2)nn1] | 1-(3- (hydroxymethyl)oxetan-3- yl)-N-((5-phenyl-1,3,4- thiadiazol-2-yl)methyl)-1H- 1,2,3-triazole-4- carboxamide | 373 |
| Example 159 | 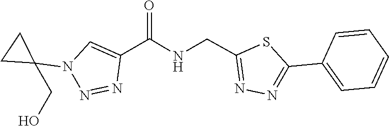 [SMILES: O=C(NCc1nnc(-c2ccccc2)s1)c1cn(C2(CO)CC2)nn1] | 1-(1- (hydroxymethyl)cyclopropyl)- N-((5-phenyl-1,3,4- thiadiazol-2-yl)methyl)-1H- 1,2,3-triazole-4- carboxamide | 357 |
| Example 160 | 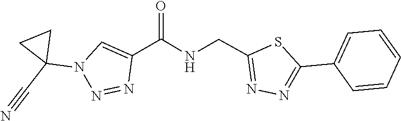 [SMILES: N#CC1(n2cc(C(=O)NCc3nnc(-c4ccccc4)s3)nn2)CC1] | 1-(1-cyanocyclopropyl)-N- ((5-phenyl-1,3,4-thiadiazol- 2-yl)methyl)-1H-1,2,3- triazole-4-carboxamide | 352 |
| Example 161 | 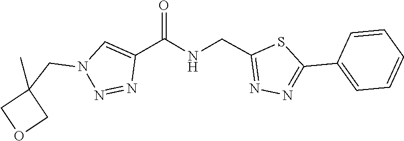 [SMILES: CC1(Cn2cc(C(=O)NCc3nnc(-c4ccccc4)s3)nn2)COC1] | 1-((3-methyloxetan-3- yl)methyl)-N-((5-phenyl- 1,3,4-thiadiazol-2- yl)methyl)-1H-1,2,3- triazole-4-carboxamide | 371 |
| Example 162 | 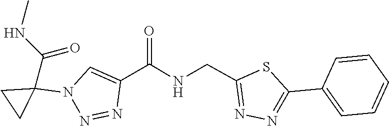 [SMILES: CNC(=O)C1(n2cc(C(=O)NCc3nnc(-c4ccccc4)s3)nn2)CC1] | 1-(1- (methylcarbamoyl) cyclopropyl)- N-((5-phenyl-1,3,4- thiadiazol-2-yl)methyl)-1 H-1,2,3-triazole-4- carboxamide | 384 |
| Example 163 | 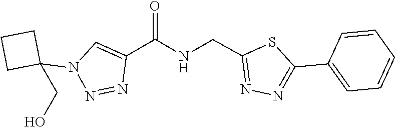 [SMILES: O=C(NCc1nnc(-c2ccccc2)s1)c1cn(C2(CO)CCC2)nn1] | 1-(1- (hydroxymethyl)cyclobutyl)- N-((5-phenyl-1,3,4- thiadiazol-2-yl)methyl)-1H- 1,2,3-triazole-4- carboxamide | 371 |
| Example 164 | 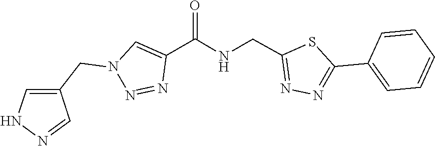 [SMILES: O=C(NCc1nnc(-c2ccccc2)s1)c1cn(Cc2cn[nH]c2)nn1] | 1-((1H-pyrazol-4-yl) methyl)-N-((5-phenyl-1,3,4- thiadiazol-2-yl)methyl)-1H- 1,2,3-triazole-4- carboxamide | 367 |
| Example 165 | 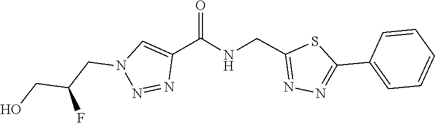 [SMILES: O=C(NCc1nnc(-c2ccccc2)s1)c1cn(C[C@@H](F)CO)nn1] | (R)-1-(2-fluoro-3- hydroxypropyl)-N-((5- phenyl-1,3,4-thiadiazol-2- yl)methyl)-1H-1,2,3- triazole-4-carboxamide | 363 |
| Example 166 | 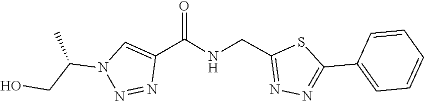 [SMILES: C[C@@H](CO)n1cc(C(=O)NCc2nnc(-c3ccccc3)s2)nn1] | (S)-1-(1-hydroxypropan-2- yl)-N-((5-phenyl-1,3,4- thiadiazol-2-yl)methyl)-1H- 1,2,3-triazole-4- carboxamide | 345 |
| Example 167 | 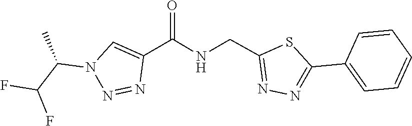 [SMILES: C[C@@H](C(F)F)n1cc(C(=O)NCc2nnc(-c3ccccc3)s2)nn1] | (S)-1-(1,1-difluoropropan- 2-yl)-N-((5-phenyl-1,3,4- thiadiazol-2-yl)methyl)-1H- 1,2,3-triazole-4- carboxamide | 365 |
| Example 168 | 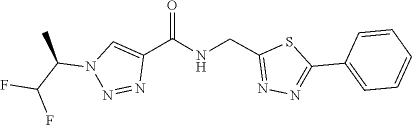 [SMILES: C[C@H](C(F)F)n1cc(C(=O)NCc2nnc(-c3ccccc3)s2)nn1] | (R)-1-(1,1-difluoropropan- 2-yl)-N-((5-phenyl-1,3,4- thiadiazol-2-yl)methyl)-1H- 1,2,3-triazole-4- carboxamide | 365 |
| Example 169 | 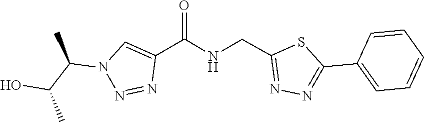 [SMILES: C[C@H](O)[C@@H](C)n1cc(C(=O)NCc2nnc(-c3ccccc3)s2)nn1] | 1-((2R,3S)-3-hydroxybutan- 2-yl)-N-((5-phenyl-1,3,4- thiadiazol-2-yl)methyl)-1H- 1,2,3-triazole-4- carboxamide | 359 |
| Example 170 | 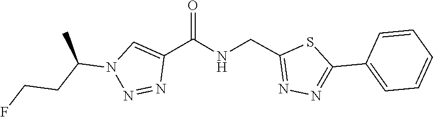 [SMILES: C[C@H](CCF)n1cc(C(=O)NCc2nnc(-c3ccccc3)s2)nn1] | (R)-1-(4-fluorobutan-2-yl)- N-((5-phenyl-1,3,4- thiadiazol-2-yl)methyl)-1H- 1,2,3-triazole-4- carboxamide | 361 |
| Example 171 | 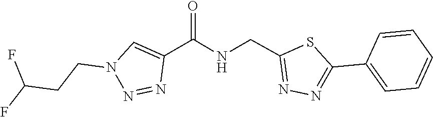 [SMILES: O=C(NCc1nnc(-c2ccccc2)s1)c1cn(CCC(F)F)nn1] | 1-(3,3-difluoropropyl)-N- ((5-phenyl-1,3,4-thiadiazol- 2-yl)methyl)-1H-1,2,3- triazole-4-carboxamide | 365 |
| Example 172 | 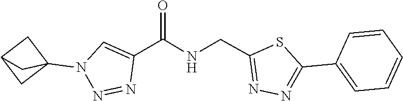 [SMILES: O=C(NCc1nnc(-c2ccccc2)s1)c1cn(C23CC(C2)C3)nn1] | 1-(bicyclo[1.1.1]pentan-1- yl)-N-((5-phenyl-1,3,4- thiadiazol-2-yl)methyl)-1H- 1,2,3-triazole-4- carboxamide | 353 |
| Example 173 | 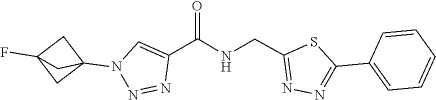 [SMILES: O=C(NCc1nnc(-c2ccccc2)s1)c1cn(C23CC(F)(C2)C3)nn1] | 1-(3- fluorobicyclo[1.1.1]pentan- 1-yl)-N-((5-phenyl-1,3,4- thiadiazol-2-yl)methyl)-1H- 1,2,3-triazole-4- carboxamide | 371 |
| Example 174 | 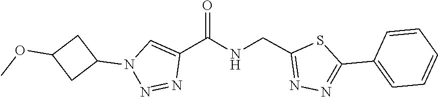 [SMILES: COC1CC(n2cc(C(=O)NCc3nnc(-c4ccccc4)s3)nn2)C1] | 1-(3-methoxycyclobutyl)- N-((5-phenyl-1,3,4- thiadiazol-2-yl)methyl)-1H- 1,2,3-triazole-4- carboxamide | 371 |

### Separation of Racemic 1-(2,2-difluorocyclopropyl)-N-((5-phenyl-1,3,4-thiadiazol-2-yl)methyl)-1H-1,2,3-triazole-4-carboxamide, Examples 130 and 131

The racemic mixture of 1-(2,2-difluorocyclopropyl)-N-((5-phenyl-1,3,4-thiadiazol-2-yl)methyl)-1H-1,2,3-triazole-4-carboxamide was resolved by chiral SFC purification (OJ-H, 21×250 mm column, 35% MeOH w/0.1% NH₄OH as cosolvent).

### Separation of Racemic cis-1-(2-methylcyclopropyl)-N-((5-phenyl-1,3,4-thiadiazol-2-yl)methyl)-1H-1,2,3-triazole-4-carboxamide, Examples 132 and 133

The racemic mixture of cis-1-(2-methylcyclopropyl)-N-((5-phenyl-1,3,4-thiadiazol-2-yl)methyl)-1H-1,2,3-triazole-4-carboxamide was resolved by chiral SFC purification (CCO F4, 21×250 mm column, 30% MeOH w/0.1% NH₄OH as cosolvent).

### Separation of Racemic 1-(3,3-difluoro-2-hydroxypropyl)-N-((5-phenyl-1,3,4-thiadiazol-2-yl)methyl)-1H-1,2,3-triazole-4-carboxamide, Examples 138 and 139

The racemic mixture of 1-(3,3-difluoro-2-hydroxypropyl)-N-((5-phenyl-1,3,4-thiadiazol-2-yl)methyl)-1H-1,2,3-triazole-4-carboxamide was resolved by chiral SFC purification (Lux-3, 21×250 mm column, 20% MeOH w/0.1% NH₄OH as cosolvent).

### Separation of Racemic trans-1-(2-(hydroxymethyl)cyclopropyl)-N-((5-phenyl-1,3,4-thiadiazol-2-yl)methyl)-1H-1,2,3-triazole-4-carboxamide, Examples 141 and 142

The racemic mixture of trans-1-(2-(hydroxymethyl)cyclopropyl)-N-((5-phenyl-1,3,4-thiadiazol-2-yl)methyl)-1H-1,2,3-triazole-4-carboxamide was resolved by chiral SFC purification (CCO F4 21×250 mm column, 40% IPA w/0.1% NH₄OH as cosolvent).

### Separation of Racemic trans-1-(-2-(difluoromethyl)cyclopropyl]-N-[(5-phenyl-1,3,4-thiadiazol-2-yl)methyl]-1H-1,2,3-triazole-4-carboxamide, Examples 143 and 144

The racemic mixture of trans-1-(-2-(difluoromethyl)cyclopropyl]-N-[(5-phenyl-1,3,4-thiadiazol-2-yl)methyl]-1H-1,2,3-triazole-4-carboxamide was resolved by chiral SFC purification (IZ 21×250 mm column, 35% MeOH w/0.1% NH₄OH as cosolvent).

### Separation of cis-1-(-2-(difluoromethyl)cyclopropyl]-N-[(5-phenyl-1,3,4-thiadiazol-2-yl)methyl]-1H-1,2,3-triazole-4-carboxamide, Examples 145 and 146

The isomeric mixture of 1 cis-1-(-2-(difluoromethyl)cyclopropyl]-N-[(5-phenyl-1,3,4-thiadiazol-2-yl)methyl]-1H-1,2,3-triazole-4-carboxamide was resolved by chiral SFC purification (CCO F4 21×250 mm column, 30% MeOH w/0.1% NH₄OH as cosolvent).

### Example 175

### Preparation of 1-(3,3-bis(hydroxymethyl)cyclobutyl)-N-((5-phenyl-1,3,4-thiadiazol-2-yl)methyl)-1H-1,2,3-triazole-4-carboxamide

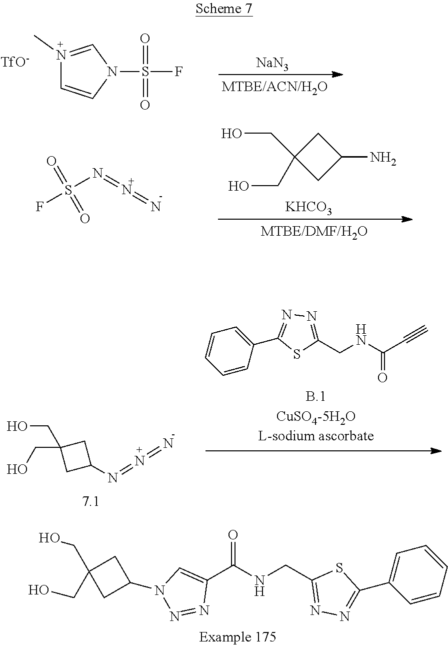

### Step 1. Preparation of Fluorosulfuryl Azide Stock Solution

Following a published procedure (Meng, G.; Guo, T.; Ma, T.; Zhang, J.; Shen, Y.; Sharpless, K. B.; Dong, J. Nature 2019, 574, 86-89), a stock solution of fluorosulfuryl azide was prepared. A 100-mL cylindrical plastic bottle was charged with aqueous NaN₃ solution (29.6 mL, 14.8 mmol, 0.5 M) and MBTE (29.6 mL). 1-(fluorosulfonyl)-3-methyl-1H-imidazol-3-ium trifluoromethanesulfonate (5.8 g, 17.7 mmol) was dissolved in MeCN (1.5 mL), and the resultant viscous solution was added rapidly to the solution of NaN₃ at 0° C. (ice-water bath). This was followed by a rinse of the vial used for preparing the solution of 1-(fluorosulfonyl)-3-methyl-1H-imidazol-3-ium trifluoromethanesulfonate with additional MeCN (1.5 mL), which was also added to the reaction mixture. The reaction mixture was stirred vigorously for 10 min in the loosely sealed plastic bottle, then the mixture was poured into a glass separating funnel. The mixture was kept in the funnel at room temperature for 30 min for phase separation. The organic phase was separated from the aqueous phase, and this organic phase was kept in a loosely sealed plastic bottle at room temperature for at least 12 h. The residual aqueous phase (approximately 1 mL in volume), which developed during the 12-hour resting period, was removed with a plastic pipette. The organic phase could be used as a solution of FSO₂N₃ in MTBE without further purification.

### Step 2. Preparation of (3-azidocyclobutane-1,1-diyl)dimethanol

An 8 mL vial was charged with (3-aminocyclobutane-1,1-diyl)dimethanol (7.9 mg, 0.060 mmol) dissolved in DMSO (100 μL), followed by FSO₂N₃ stock solution (180 μL, 0.060 mmol, approximately 0.2 M in DMF/MTBE 1:1 v/v, made by diluting 200 μL of above stock with 200 μL DMF) and aqueous KHCO₃ (80 μL, 0.240 mmol, 3 M). The reaction mixture was stirred for 16 h at room temperature. After completion, EtOAc (2 mL) was added and the mixture was washed sequentially with brine (2×2 mL), water (2×2 mL) and brine (2×2 mL), dried over MgSO₄, and concentrated to afford (3-azidocyclobutane-1,1-diyl)dimethanol which was used directly in the next step.

### Step 3. Preparation of 1-(3,3-bis(hydroxymethyl)cyclobutyl)-N-((5-phenyl-1,3,4-thiadiazol-2-yl)methyl)-1H-1,2,3-triazole-4-carboxamide

In a glovebox a vial was charged with copper(II) sulfate pentahydrate (1.49 mg, 6 μmol), L-ascorbic acid sodium salt (2.37 mg, 12 μmol) and N-((5-phenyl-1,3,4-thiadiazol-2-yl)methyl)propiolamide (14.6 mg, 60 μmol), followed by a solution of (3-azidocyclobutane-1,1-diyl)dimethanol in DMF (600 μL). The vial was capped and stirred at 30° C. for 16 hours. The crude was diluted with DMSO (500 μL), filtered and purified via reverse phase HPLC (eluting acetonitrile in water, with TFA modifier) to afford 1-(3,3-bis(hydroxymethyl)cyclobutyl)-N-((5-phenyl-1,3,4-thiadiazol-2-yl)methyl)-1H-1,2,3-triazole-4-carboxamide. as a mixture of cis/trans-isomers. LC/MS (m/z): 401 (M+H)⁺.

Examples shown in Example Table 7 below, were prepared according to procedures analogous to those outlined in Example 175 and General Scheme 3 above using the appropriate starting materials obtained from methods described above or elsewhere, or as obtained from commercial sources.

EXAMPLE TABLE 7

| Example | Structure | Name | Mass [M + H]⁺ |
|---|---|---|---|
| Example 176 | 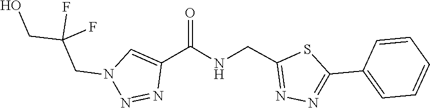 [SMILES: O=C(NCc1nnc(-c2ccccc2)s1)c1cn(CC(F)(F)CO)nn1] | 1-(2,2-difluoro-3- hydroxypropyl)-N-((5- phenyl-1,3,4-thiadiazol-2- yl)methyl)-1H-1,2,3- triazole-4-carboxamide | 381 |
| Example 177 | 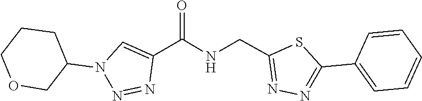 [SMILES: O=C(NCc1nnc(-c2ccccc2)s1)c1cn(C2CCCOC2)nn1] | N-((5-phenyl-1,3,4- thiadiazol-2-yl)methyl)-1- (tetrahydro-2H-pyran-3-yl)- 1H-1,2,3-triazole-4- carboxamide | 371 |
| Example 178 | 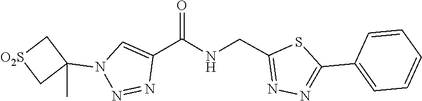 [SMILES: CC1(n2cc(C(=O)NCc3nnc(-c4ccccc4)s3)nn2)CS(=O)(=O)C1] | 1-(3-methyl-1,1- dioxidothietan-3-yl)-N-((5- phenyl-1,3,4-thiadiazol-2- yl)methyl)-1H-1,2,3- triazole-4-carboxamide | 405 |
| Example 179 | 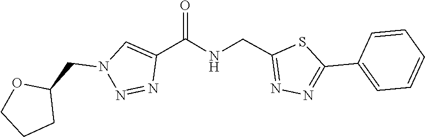 [SMILES: O=C(NCc1nnc(-c2ccccc2)s1)c1cn(C[C@H]2CCCO2)nn1] | (R)-N-((5-phenyl-1,3,4- thiadiazol-2-yl)methyl)-1- ((tetrahydrofuran-2- yl)methyl)-1H-1,2,3- triazole-4-carboxamide | 371 |
| Example 180 | 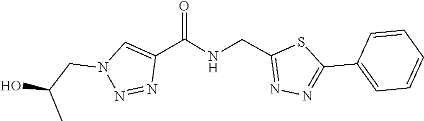 [SMILES: C[C@@H](O)Cn1cc(C(=O)NCc2nnc(-c3ccccc3)s2)nn1] | (R)-1-(2-hydroxypropyl)-N- ((5-phenyl-1,3,4-thiadiazol- 2-yl)methyl)-1H-1,2,3- triazole-4-carboxamide | 345 |
| Example 181 | 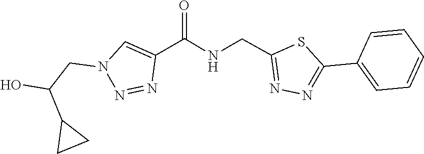 [SMILES: O=C(NCc1nnc(-c2ccccc2)s1)c1cn(CC(O)C2CC2)nn1] | 1-(2-cyclopropyl-2- hydroxyethyl)-N-((5- phenyl-1,3,4-thiadiazol-2- yl)methyl)-1H-1,2,3- triazole-4-carboxamide | 371 |
| Example 182 | 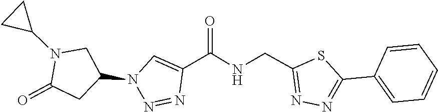 | (R)-1-(1-cyclopropyl-5- oxopyrrolidin-3-yl)-N-((5- phenyl-1,3,4-thiadiazol-2- yl)methyl)-1H-1,2,3- triazole-4-carboxamide | 410 |
| Example 183 | 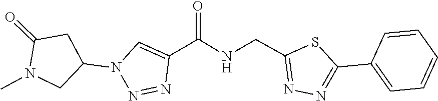 [SMILES: CN1CC(n2cc(C(=O)NCc3nnc(-c4ccccc4)s3)nn2)CC1=O] | 1-(1-methyl-5- oxopyrrolidin-3-yl)-N-((5- phenyl-1,3,4-thiadiazol-2- yl)methyl)-1H-1,2,3- triazole-4-carboxamide | 384 |
| Example 184 | 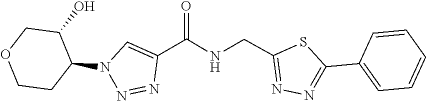 [SMILES: O=C(NCc1nnc(-c2ccccc2)s1)c1cn([C@H]2CCOC[C@@H]2O)nn1] | 1-((3R,4S)-3- hydroxytetrahydro-2H- pyran-4-yl)-N-((5-phenyl- 1,3,4-thiadiazol-2- yl)methyl)-1H-1,2,3- triazole-4-carboxamide | 387 |
| Example 185 | 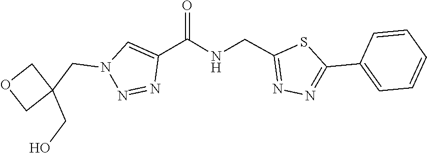 [SMILES: O=C(NCc1nnc(-c2ccccc2)s1)c1cn(CC2(CO)COC2)nn1] | 1-((3- (hydroxymethyl)oxetan-3- yl)methyl)-N-((5-phenyl- 1,3,4-thiadiazol-2- yl)methyl)-1H-1,2,3- triazole-4-carboxamide | 387 |
| Example 186 | 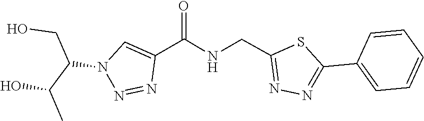 | 1-((2S,3S)-1,3- dihydroxybutan-2-yl)-N- ((5-phenyl-1,3,4-thiadiazol- 2-yl)methyl)-1H-1,2,3- triazole-4-carboxamide | 375 |
| Example 187 | 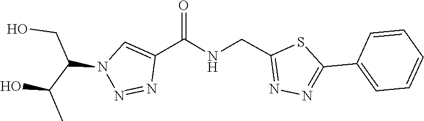 | 1-((2S,3R)-1,3- dihydroxybutan-2-yl)-N- ((5-phenyl-1,3,4-thiadiazol- 2-yl)methyl)-1H-1,2,3- triazole-4-carboxamide | 375 |
| Example 188 | 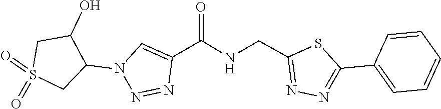 [SMILES: O=C(NCc1nnc(-c2ccccc2)s1)c1cn(C2CS(=O)(=O)CC2O)nn1] | 1-(4-hydroxy-1,1- dioxidotetrahydrothiophen- 3-yl)-N-((5-phenyl-1,3,4- thiadiazol-2-yl)methyl)-1H- 1,2,3-triazole-4- carboxamide | 421 |
| Example 189 | 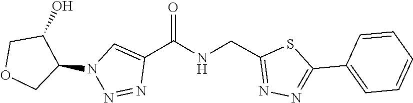 [SMILES: O=C(NCc1nnc(-c2ccccc2)s1)c1cn([C@H]2COC[C@@H]2O)nn1] | 1-((3S,4R)-4- hydroxytetrahydrofuran-3- yl)-N-((5-phenyl-1,3,4- thiadiazol-2-yl)methyl)-1H- 1,2,3-triazole-4- carboxamide | 373 |
| Example 190 | 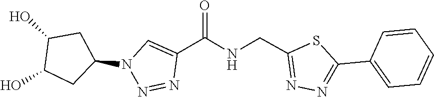 [SMILES: O=C(NCc1nnc(-c2ccccc2)s1)c1cn([C@@H]2C[C@@H](O)[C@@H](O)C2)nn1] | 1-((1s,3R,4S)-3,4- dihydroxycyclopentyl)-N- ((5-phenyl-1,3,4-thiadiazol- 2-yl)methyl)-1H-1,2,3- triazole-4-carboxamide | 387 |
| Example 191 | 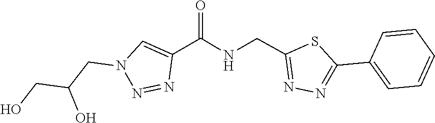 [SMILES: O=C(NCc1nnc(-c2ccccc2)s1)c1cn(CC(O)CO)nn1] | 1-(2,3-dihydroxypropyl)-N- ((5-phenyl-1,3,4-thiadiazol- 2-yl)methyl)-1H-1,2,3- triazole-4-carboxamide | 361 |
| Example 192 | 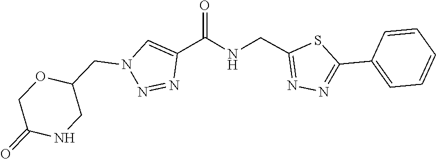 [SMILES: O=C1COC(Cn2cc(C(=O)NCc3nnc(-c4ccccc4)s3)nn2)CN1] | 1-((5-oxomorpholin-2- yl)methyl)-N-((5-phenyl- 1,3,4-thiadiazol-2- yl)methyl)-1H-1,2,3- triazole-4-carboxamide | 400 |
| Example 193 | 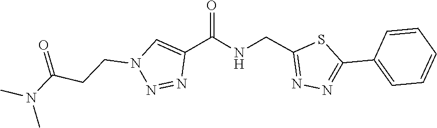 [SMILES: CN(C)C(=O)CCn1cc(C(=O)NCc2nnc(-c3ccccc3)s2)nn1] | 1-(3-(dimethylamino)-3- oxopropyl)-N-((5-phenyl- 1,3,4-thiadiazol-2- yl)methyl)-1H-1,2,3- triazole-4-carboxamide | 386 |
| Example 194 | 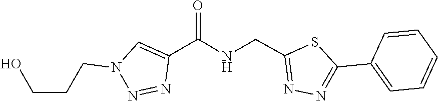 [SMILES: O=C(NCc1nnc(-c2ccccc2)s1)c1cn(CCCO)nn1] | 1-(3-hydroxypropyl)-N-((5- phenyl-1,3,4-thiadiazol-2- yl)methyl)-1H-1,2,3- triazole-4-carboxamide | 345 |
| Example 195 | 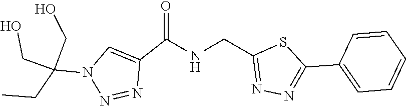 [SMILES: CCC(CO)(CO)n1cc(C(=O)NCc2nnc(-c3ccccc3)s2)nn1] | 1-(1-hydroxy-2- (hydroxymethyl)butan-2- yl)-N-((5-phenyl-1,3,4- thiadiazol-2-yl)methyl)-1H- 1,2,3-triazole-4- carboxamide | 389 |
| Example 196 | 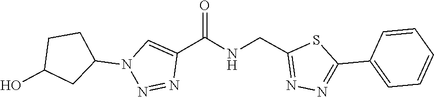 [SMILES: O=C(NCc1nnc(-c2ccccc2)s1)c1cn(C2CCC(O)C2)nn1] | 1-(3-hydroxycyclopentyl)- N-((5-phenyl-1,3,4- thiadiazol-2-yl)methyl)-1H- 1,2,3-triazole-4- carboxamide | 371 |
| Example 197 | 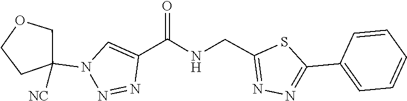 [SMILES: N#CC1(n2cc(C(=O)NCc3nnc(-c4ccccc4)s3)nn2)CCOC1] | 1-(3-cyanotetrahydrofuran- 3-yl)-N-((5-phenyl-1,3,4- thiadiazol-2-yl)methyl)-1H- 1,2,3-triazole-4- carboxamide | 382 |
| Example 198 | 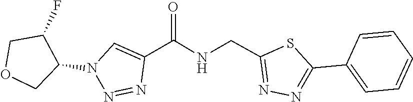 [SMILES: O=C(NCc1nnc(-c2ccccc2)s1)c1cn([C@@H]2COC[C@@H]2F)nn1] | 1-((3R,4R)-4- fluorotetrahydrofuran-3-yl)- N-((5-phenyl-1,3,4- thiadiazol-2-yl)methyl)-1H- 1,2,3-triazole-4- carboxamide | 375 |
| Example 199 | 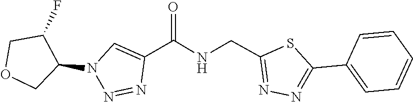 [SMILES: O=C(NCc1nnc(-c2ccccc2)s1)c1cn([C@H]2COC[C@@H]2F)nn1] | 1-((3S,4R)-4- fluorotetrahydrofuran-3-yl)- N-((5-phenyl-1,3,4- thiadiazol-2-yl)methyl)-1H- 1,2,3-triazole-4- carboxamide | 375 |
| Example 200 | 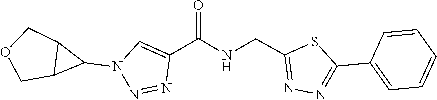 [SMILES: O=C(NCc1nnc(-c2ccccc2)s1)c1cn(C2C3COCC32)nn1] | 1-(3- oxabicyclo[3.1.0]hexan-6- yl)-N-((5-phenyl-1,3,4- thiadiazol-2-yl)methyl)-1H- 1,2,3-triazole-4- carboxamide | 369 |
| Example 201 | 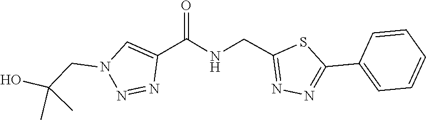 [SMILES: CC(C)(O)Cn1cc(C(=O)NCc2nnc(-c3ccccc3)s2)nn1] | 1-(2-hydroxy-2- methylpropyl)-N-((5- phenyl-1,3,4-thiadiazol-2- yl)methyl)-1H-1,2,3- triazole-4-carboxamide | 359 |
| Example 202 | 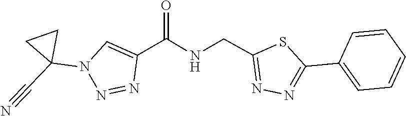 [SMILES: N#CC1(n2cc(C(=O)NCc3nnc(-c4ccccc4)s3)nn2)CC1] | 1-(1-cyanocyclopropyl)-N- ((5-phenyl-1,3,4-thiadiazol- 2-yl)methyl)-1H-1,2,3- triazole-4-carboxamide | 352 |
| Example 203 | 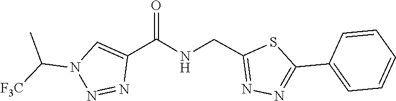 [SMILES: CC(n1cc(C(=O)NCc2nnc(-c3ccccc3)s2)nn1)C(F)(F)F] | N-((5-phenyl-1,3,4- thiadiazol-2-yl)methyl)-1- (1,1,1-trifluoropropan-2- yl)-1H-1,2,3-triazole-4- carboxamide | 383 |
| Example 204 | 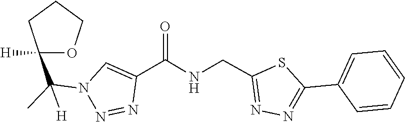 [SMILES: CC([C@H]1CCCO1)n1cc(C(=O)NCc2nnc(-c3ccccc3)s2)nn1] | N-((5-phenyl-1,3,4- thiadiazol-2-yl)methyl)-1- (1-((R)-tetrahydrofuran-2- yl)ethyl)-1H-1,2,3-triazole- 4-carboxamide | 385 |
| Example 205 | 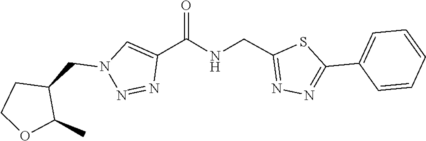 [SMILES: C[C@H]1OCC[C@H]1Cn1cc(C(=O)NCc2nnc(-c3ccccc3)s2)nn1] | 1-(((2R,3S)-2- methyltetrahydrofuran-3- yl)methyl)-N-((5-phenyl- 1,3,4-thiadiazol-2- yl)methyl)-1H-1,2,3- triazole-4-carboxamide | 385 |
| Example 206 | 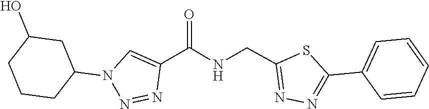 [SMILES: O=C(NCc1nnc(-c2ccccc2)s1)c1cn(C2CCCC(O)C2)nn1] | 1-(3-hydroxycyclohexyl)- N-((5-phenyl-1,3,4- thiadiazol-2-yl)methyl)-1H- 1,2,3-triazole-4- carboxamide | 385 |
| Example 207 | 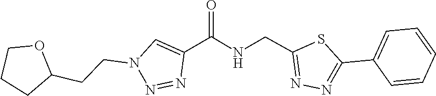 [SMILES: O=C(NCc1nnc(-c2ccccc2)s1)c1cn(CCC2CCCO2)nn1] | N-((5-phenyl-1,3,4- thiadiazol-2-yl)methyl)-1- (2-(tetrahydrofuran-2- yl)ethyl)-1H-1,2,3-triazole- 4-carboxamide | 385 |
| Example 208 | 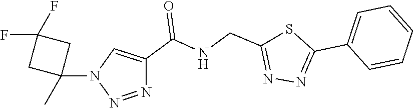 [SMILES: CC1(n2cc(C(=O)NCc3nnc(-c4ccccc4)s3)nn2)CC(F)(F)C1] | 1-(3,3-difluoro-1- methylcyclobutyl)-N-((5- phenyl-1,3,4-thiadiazol-2- yl)methyl)-1H-1,2,3- triazole-4-carboxamide | 391 |
| Example 209 | 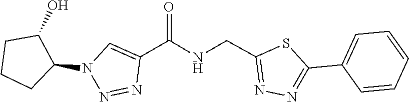 [SMILES: O=C(NCc1nnc(-c2ccccc2)s1)c1cn([C@H]2CCC[C@@H]2O)nn1] | 1-((1S,2S)-2- hydroxycyclopentyl)-N-((5- phenyl-1,3,4-thiadiazol-2- yl)methyl)-1H-1,2,3- triazole-4-carboxamide | 371 |
| Example 210 | 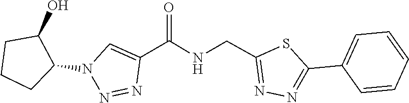 [SMILES: O=C(NCc1nnc(-c2ccccc2)s1)c1cn([C@@H]2CCC[C@H]2O)nn1] | 1-((1R,2R)-2- hydroxycyclopentyl)-N-((5- phenyl-1,3,4-thiadiazol-2- yl)methyl)-1H-1,2,3- triazole-4-carboxamide | 371 |
| Example 211 | 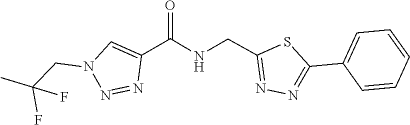 [SMILES: CC(F)(F)Cn1cc(C(=O)NCc2nnc(-c3ccccc3)s2)nn1] | 1-(2,2-difluoropropyl)-N- ((5-phenyl-1,3,4-thiadiazol- 2-yl)methyl)-1H-1,2,3- triazole-4-carboxamide | 365 |
| Example 212 | 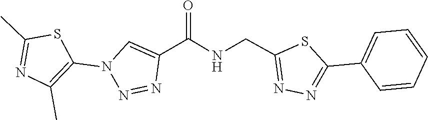 [SMILES: Cc1nc(C)c(-n2cc(C(=O)NCc3nnc(-c4ccccc4)s3)nn2)s1] | 1-(2,4-dimethylthiazol-5- yl)-N-((5-phenyl-1,3,4- thiadiazol-2-yl)methyl)-1H- 1,2,3-triazole-4- carboxamide | 398 |

### Example 213

### Preparation of N-((5-phenyl-1,3,4-thiadiazol-2-yl)methyl)-1-(2,2,2-trifluoroethyl)-1H-1,2,3-triazole-4-carboxamide

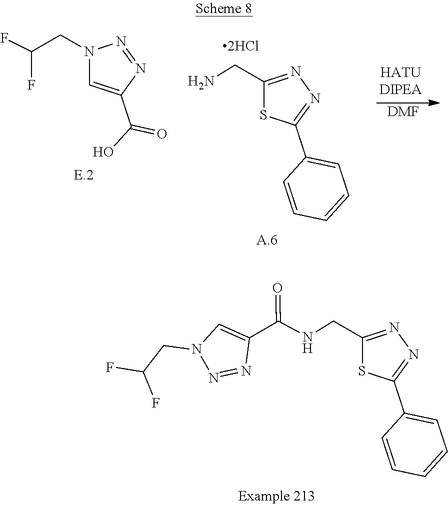

HATU (5.15 g, 13.6 mmol) was added to a solution of 1-(2,2-difluoroethyl)-1H-1,2,3-triazole-4-carboxylic acid (2.00 g, 11.3 mmol), (5-phenyl-1,3,4-thiadiazol-2-yl)methanamine dihydrochloride (3.28 g, 12.4 mmol) and DIPEA (5.92 mL, 33.9 mmol) in DMF (100 mL) at room temperature and the reaction was stirred overnight. The reaction was quenched with saturated NaHCO₃ solution (100 mL) and stirred at room temperature for 10 minutes. The solid was filtered and washed with saturated NaHCO₃ solution (3×50 mL), followed by distilled water (3×50 mL) and dried on the frit under vacuum with a stream of nitrogen blowing over top. The crude product was dissolved in 3:1 EtOAc/EtOH (approximately 500 mL) and absorbed on silica gel. The crude was purified by silica gel chromatography (eluting 3:1 EtOAc/EtOH in hexanes) to afford N-((5-phenyl-1,3,4-thiadiazol-2-yl)methyl)-1-(2,2,2-trifluoroethyl)-1H-1,2,3-triazole-4-carboxamide. LC/MS (m/z): 351 (M+H)⁺. ¹H NMR (499 MHz, DMSO) δ 9.69-9.56 (m, 1H), 8.69 (s, 1H), 8.00-7.90 (m, 2H), 7.59-7.47 (m, 3H), 6.67-6.37 (m, 1H), 5.10-4.97 (m, 2H), 4.87 (d, J=5.7 Hz, 2H).

Examples shown in Example Table 8 below, were prepared according to procedures analogous to those outlined in Example 213 and General Scheme 2 above using the appropriate starting materials obtained from methods described above or elsewhere, or as obtained from commercial sources.

Example Table 8

| | | | Mass |
|---|---|---|---|
| Example | Structure | Name | [M + H]⁺ |
| Example 214 | 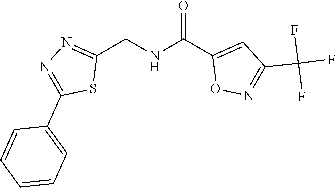 [SMILES: O=C(NCc1nnc(-c2ccccc2)s1)c1cc(C(F)(F)F)no1] | N-[(5-phenyl-1,3,4- thiadiazol-2-yl)methyl]-3- (trifluoromethyl)-1,2- oxazole-5-carboxamide | 355 |
| Example 215 | 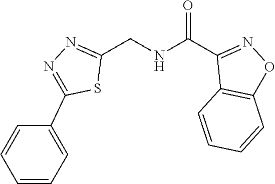 [SMILES: O=C(NCc1nnc(-c2ccccc2)s1)c1noc2ccccc12] | N-[(5-phenyl-1,3,4- thiadiazol-2-yl)methyl]-1,2- benzoxazole-3-carboxamide | 337 |
| Example 216 | 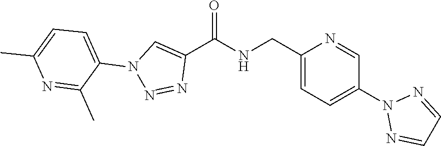 [SMILES: Cc1ccc(-n2cc(C(=O)NCc3ccc(-n4nccn4)cn3)nn2)c(C)n1] | 1-(2,6-dimethylpyridin-3- yl)-N-{[5-(2H-1,2,3-triazol- 2-yl)pyridin-2-yl]methyl}- 1H-1,2,3-triazole-4- carboxamide | 376 |
| Example 217 | 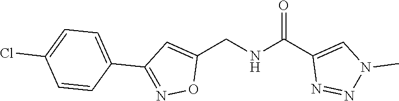 [SMILES: Cn1cc(C(=O)NCc2cc(-c3ccc(Cl)cc3)no2)nn1] | N-{[3-(4-chlorophenyl)- 1,2-oxazol-5-yl]methyl}-1- methyl-1H-1,2,3-triazole-4- carboxamide | 318 |
| Example 218 | 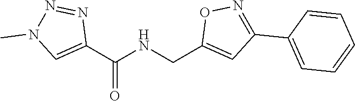 [SMILES: Cn1cc(C(=O)NCc2cc(-c3ccccc3)no2)nn1] | 1-methyl-N-[(3-phenyl-1,2- oxazol-5-yl)methyl]-1H- 1,2,3-triazole-4- carboxamide | 284 |
| Example 219 | 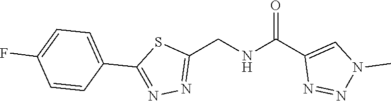 [SMILES: Cn1cc(C(=O)NCc2nnc(-c3ccc(F)cc3)s2)nn1] | N-{[5-(4-fluorophenyl)- 1,3,4-thiadiazol-2- yl]methyl}-1-methyl-1H- 1,2,3-triazole-4- carboxamide | [M + Na]⁺ 341 |
| Example 220 | 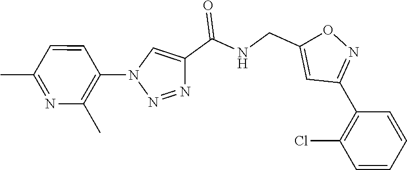 [SMILES: Cc1ccc(-n2cc(C(=O)NCc3cc(-c4ccccc4Cl)no3)nn2)c(C)n1] | N-{[3-(2-chlorophenyl)- 1,2-oxazol-5-yl]methyl}-1- (2,6-dimethylpyridin-3-yl)- 1H-1,2,3-triazole-4- carboxamide | 409 |
| Example 221 | 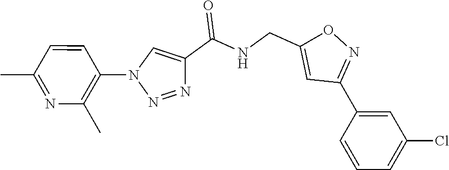 [SMILES: Cc1ccc(-n2cc(C(=O)NCc3cc(-c4cccc(Cl)c4)no3)nn2)c(C)n1] | N-{[3-(3-chlorophenyl)- 1,2-oxazol-5-yl]methyl}-1- (2,6-dimethylpyridin-3-yl)- 1H-1,2,3-triazole-4- carboxamide | 409 |
| Example 222 | 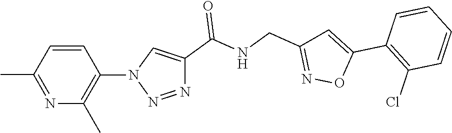 [SMILES: Cc1ccc(-n2cc(C(=O)NCc3cc(-c4ccccc4Cl)on3)nn2)c(C)n1] | N-{[5-(2-chlorophenyl)- 1,2-oxazol-3-yl]methyl}-1- (2,6-dimethylpyridin-3-yl)- 1H-1,2,3-triazole-4- carboxamide | 409 |
| Example 223 | 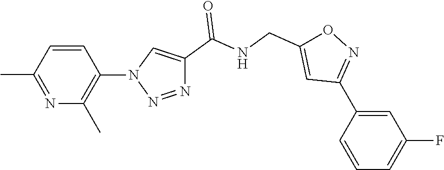 [SMILES: Cc1ccc(-n2cc(C(=O)NCc3cc(-c4cccc(F)c4)no3)nn2)c(C)n1] | 1-(2,6-dimethylpyridin-3- yl)-N-{[3-(3-fluorophenyl)- 1,2-oxazol-5-yl]methyl}- 1H-1,2,3-triazole-4- carboxamide | 393 |
| Example 224 | 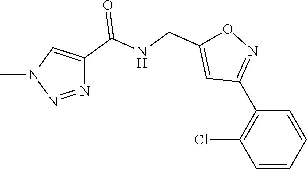 [SMILES: Cn1cc(C(=O)NCc2cc(-c3ccccc3Cl)no2)nn1] | N-{[3-(2-chlorophenyl)- 1,2-oxazol-5-yl]methyl}-1- methyl-1H-1,2,3-triazole-4- carboxamide | 318 |
| Example 225 | 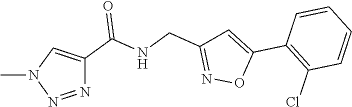 [SMILES: Cn1cc(C(=O)NCc2cc(-c3ccccc3Cl)on2)nn1] | N-{[5-(2-chlorophenyl)- 1,2-oxazol-3-yl]methyl}-1- methyl-1H-1,2,3-triazole-4- carboxamide | 318 |
| Example 226 | 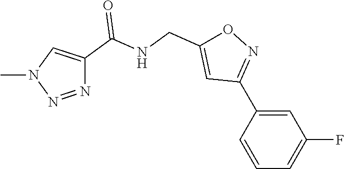 [SMILES: Cn1cc(C(=O)NCc2cc(-c3cccc(F)c3)no2)nn1] | N-{[3-(3-fluorophenyl)-1,2- oxazol-5-yl]methyl}-1- methyl-1H-1,2,3-triazole-4- carboxamide | 302 |
| Example 227 | 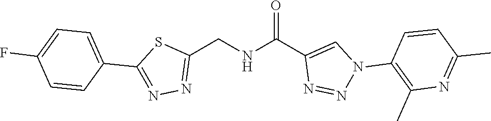 [SMILES: Cc1ccc(-n2cc(C(=O)NCc3nnc(-c4ccc(F)cc4)s3)nn2)c(C)n1] | 1-(2,6-dimethylpyridin-3- yl)-N-{[5-(4-fluorophenyl)- 1,3,4-thiadiazol-2- yl]methyl}-1H-1,2,3- triazole-4-carboxamide | 410 |
| Example 228 | 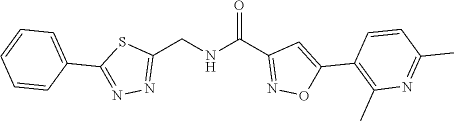 [SMILES: Cc1ccc(-c2cc(C(=O)NCc3nnc(-c4ccccc4)s3)no2)c(C)n1] | 5-(2,6-dimethylpyridin-3- yl)-N-[(5-phenyl-1,3,4- thiadiazol-2-yl)methyl]-1,2- oxazole-3-carboxamide | 392 |
| Example 229 | 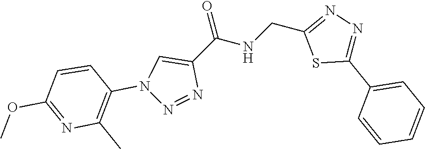 [SMILES: COc1ccc(-n2cc(C(=O)NCc3nnc(-c4ccccc4)s3)nn2)c(C)n1] | 1-(6-methoxy-2- methylpyridin-3-yl)- N-((5-phenyl-1,3,4- thiadiazol-2-yl)methyl)-1H- 1,2,3-triazole-4- carboxamide | 408 |
| Example 230 | 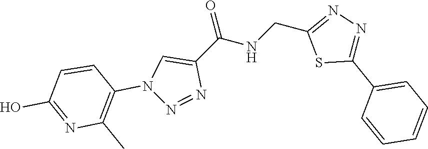 [SMILES: Cc1nc(O)ccc1-n1cc(C(=O)NCc2nnc(-c3ccccc3)s2)nn1] | 1-(6-hydroxy-2- methylpyridin-3-yl)-N-((5- phenyl-1,3,4-thiadiazol- 2-yl)methyl)- 1H-1,2,3-triazole-4- carboxamide | 394 |
| Example 231 | 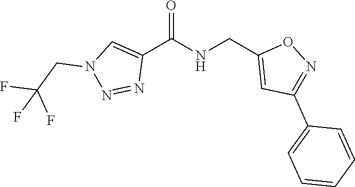 [SMILES: O=C(NCc1cc(-c2ccccc2)no1)c1cn(CC(F)(F)F)nn1] | N-((3-phenylisoxazol-5- yl)methyl)-1-(2,2,2- trifluoroethyl)-1H-1,2,3- triazole-4-carboxamide | 352 |
| Example 232 | 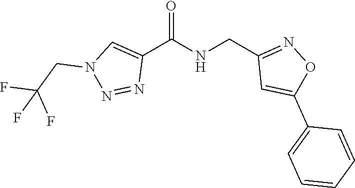 [SMILES: O=C(NCc1cc(-c2ccccc2)on1)c1cn(CC(F)(F)F)nn1] | N-((5-phenylisoxazol-3- yl)methyl)-1-(2,2,2- trifluoroethyl)-1H-1,2,3- triazole-4-carboxamide | 352 |
| Example 233 | 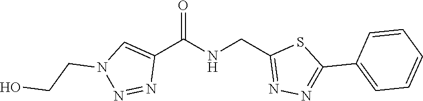 [SMILES: O=C(NCc1nnc(-c2ccccc2)s1)c1cn(CCO)nn1] | 1-(2-hydroxyethyl)-N-((5- phenyl-1,3,4-thiadiazol-2- yl)methyl)-1H-1,2,3- triazole-4-carboxamide | 331 |
| Example 234 | 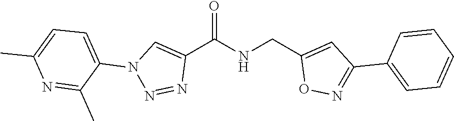 [SMILES: Cc1ccc(-n2cc(C(=O)NCc3cc(-c4ccccc4)no3)nn2)c(C)n1] | 1-(2,6-dimethylpyridin-3- yl)-N-((3-phenylisoxazol-5- yl)methyl)-1H-1,2,3- triazole-4-carboxamide | 375 |
| Example 235 | 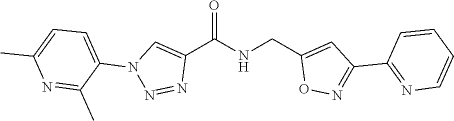 [SMILES: Cc1ccc(-n2cc(C(=O)NCc3cc(-c4ccccn4)no3)nn2)c(C)n1] | 1-(2,6-dimethylpyridin-3- yl)-N-((3-(pyridin-2- yl)isoxazol-5-yl)methyl)- 1H-1,2,3-triazole-4- carboxamide | 376 |
| Example 236 | 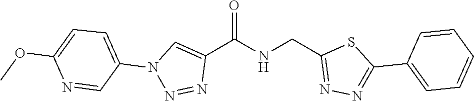 [SMILES: COc1ccc(-n2cc(C(=O)NCc3nnc(-c4ccccc4)s3)nn2)cn1] | 1-(6-methoxypyridin-3-yl)- N-((5-phenyl-1,3,4- thiadiazol-2-yl)methyl)-1H- 1,2,3-triazole-4- carboxamide | 394 |
| Example 237 | 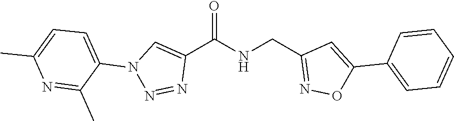 [SMILES: Cc1ccc(-n2cc(C(=O)NCc3cc(-c4ccccc4)on3)nn2)c(C)n1] | 1-(2,6-dimethylpyridin-3- yl)-N-((5-phenylisoxazol-3- yl)methyl)-1H-1,2,3- triazole-4-carboxamide | 375 |
| Example 238 | 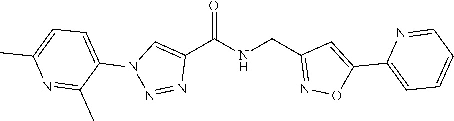 [SMILES: Cc1ccc(-n2cc(C(=O)NCc3cc(-c4ccccn4)on3)nn2)c(C)n1] | 1-(2,6-dimethylpyridin-3- yl)-N-((5-(pyridin-2- yl)isoxazol-3-yl)methyl)- 1H-1,2,3-triazole-4- carboxamide | 376 |
| Example 239 | 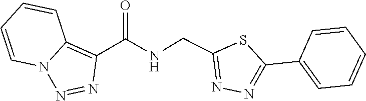 [SMILES: O=C(NCc1nnc(-c2ccccc2)s1)c1nnn2ccccc12] | N-((5-phenyl-1,3,4- thiadiazol-2-yl)methyl)- [1,2,3]triazolo[1,5- a]pyridine-3-carboxamide | 337 |
| Example 240 | 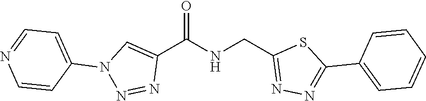 [SMILES: O=C(NCc1nnc(-c2ccccc2)s1)c1cn(-c2ccncc2)nn1] | N-((5-phenyl-1,3,4- thiadiazol-2-yl)methyl)-1- (pyridin-4-yl)-1H-1,2,3- triazole-4-carboxamide | 364 |
| Example 241 | 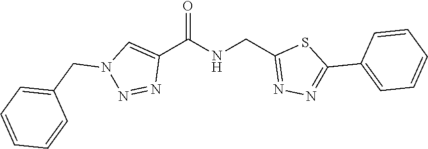 [SMILES: O=C(NCc1nnc(-c2ccccc2)s1)c1cn(Cc2ccccc2)nn1] | 1-benzyl-N-((5-phenyl- 1,3,4-thiadiazol-2- yl)methyl)-1H-1,2,3- triazole-4-carboxamide | 377 |
| Example 242 | 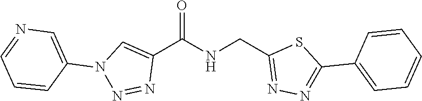 [SMILES: O=C(NCc1nnc(-c2ccccc2)s1)c1cn(-c2cccnc2)nn1] | N-((5-phenyl-1,3,4- thiadiazol-2-yl)methyl)-1- (pyridin-3-yl)-1H-1,2,3- triazole-4-carboxamide | 364 |
| Example 243 | 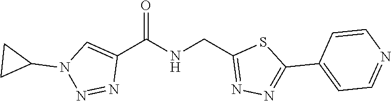 [SMILES: O=C(NCc1nnc(-c2ccncc2)s1)c1cn(C2CC2)nn1] | 1-cyclopropyl-N-((5- (pyridin-4-yl)-1,3,4- thiadiazol-2-yl)methyl)-1H- 1,2,3-triazole-4- carboxamide | 328 |
| Example 244 | 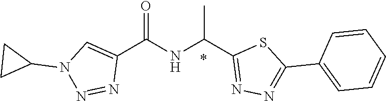 [SMILES: CC(NC(=O)c1cn(C2CC2)nn1)c1nnc(-c2ccccc2)s1] | 1-cyclopropyl-N-(1-(5- phenyl-1,3,4-thiadiazol-2- yl)ethyl)-1H-1,2,3-triazole- 4-carboxamide, peak 1 | 341 |
| Example 245 | 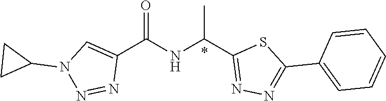 [SMILES: CC(NC(=O)c1cn(C2CC2)nn1)c1nnc(-c2ccccc2)s1] | 1-cyclopropyl-N-(1-(5- phenyl-1,3,4-thiadiazol-2- yl)ethyl)-1H-1,2,3-triazole- 4-carboxamide, peak 2 | 341 |
| Example 246 | 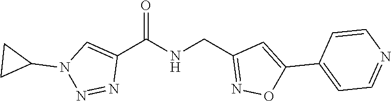 [SMILES: O=C(NCc1cc(-c2ccncc2)on1)c1cn(C2CC2)nn1] | 1-cyclopropyl-N-((5- (pyridin-4-yl)isoxazol-3- yl)methyl)-1H-1,2,3- triazole-4-carboxamide | 311 |
| Example 247 | 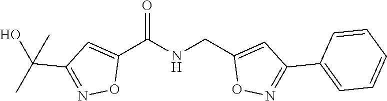 [SMILES: CC(C)(O)c1cc(C(=O)NCc2cc(-c3ccccc3)no2)on1] | 3-(2-hydroxypropan-2-yl)- N-((3-phenylisoxazol-5- yl)methyl)isoxazole-5- carboxamide | 328 |
| Example 248 | 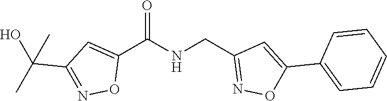 [SMILES: CC(C)(O)c1cc(C(=O)NCc2cc(-c3ccccc3)on2)on1] | 3-(2-hydroxypropan-2-yl)- N-((5-phenylisoxazol-3- yl)methyl)isoxazole-5- carboxamide | 328 |
| Example 249 | 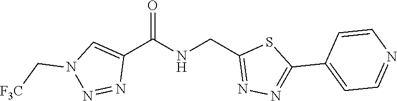 [SMILES: O=C(NCc1nnc(-c2ccncc2)s1)c1cn(CC(F)(F)F)nn1] | N-((5-(pyridin-4-yl)-1,3,4- thiadiazol-2-yl)methyl)-1- (2,2,2-trifluoroethyl)-1H- 1,2,3-triazole-4- carboxamide | 370 |
| Example 250 | 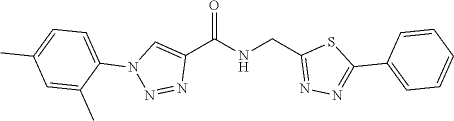 [SMILES: Cc1ccc(-n2cc(C(=O)NCc3nnc(-c4ccccc4)s3)nn2)c(C)c1] | 1-(2,4-dimethylphenyl)-N- ((5-(pyridin-4-yl)-1,3,4- thiadiazol-2-yl)methyl)-1H- 1,2,3-triazole-4- carboxamide | 391 |
| Example 251 | 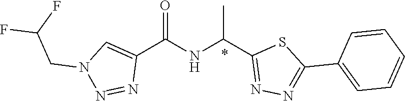 [SMILES: CC(NC(=O)c1cn(CC(F)F)nn1)c1nnc(-c2ccccc2)s1] | 1-(2,2-difluoroethyl)-N-(1- (5-phenyl-1,3,4-thiadiazol- 2-yl)ethyl)-1H-1,2,3- triazole-4-carboxamide, peak 1 | 365 |
| Example 252 | 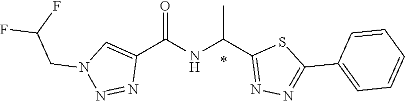 [SMILES: CC(NC(=O)c1cn(CC(F)F)nn1)c1nnc(-c2ccccc2)s1] | 1-(2,2-difluoroethyl)-N-(1- (5-phenyl-1,3,4-thiadiazol- 2-yl)ethyl)-1H-1,2,3- triazole-4-carboxamide, peak 2 | 365 |
| Example 253 | 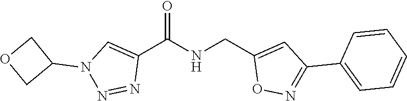 [SMILES: O=C(NCc1cc(-c2ccccc2)no1)c1cn(C2COC2)nn1] | 1-(oxetan-3-yl)-N-((3- phenylisoxazol-5- yl)methyl)-1H-1,2,3- triazole-4-carboxamide | 326 |
| Example 254 | 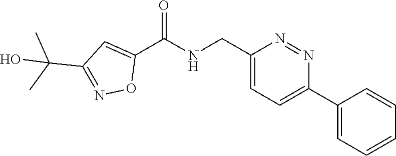 [SMILES: CC(C)(O)c1cc(C(=O)NCc2ccc(-c3ccccc3)nn2)on1] | 3-(2-hydroxypropan-2-yl)- N-((6-phenylpyridazin-3- yl)methyl)isoxazole-5- carboxamide | 339 |
| Example 255 | 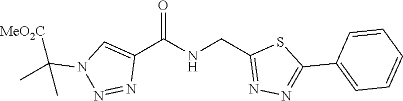 [SMILES: COC(=O)C(C)(C)n1cc(C(=O)NCc2nnc(-c3ccccc3)s2)nn1] | methyl 2-methyl-2-(4-(((5- phenyl-1,3,4-thiadiazol-2- yl)methyl)carbamoyl)-1H- 1,2,3-triazol-1- yl)propanoate | 387 |
| Example 256 | 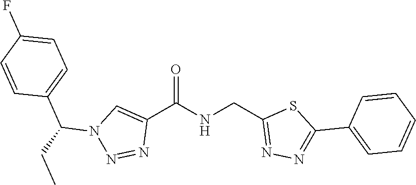 [SMILES: CC[C@H](c1ccc(F)cc1)n1cc(C(=O)NCc2nnc(-c3ccccc3)s2)nn1] | (R)-1-(1-(4- fluorophenyl)propyl)-N-((5- phenyl-1,3,4-thiadiazol-2- yl)methyl)-1H-1,2,3- triazole-4-carboxamide | 423 |
| Example 257 | 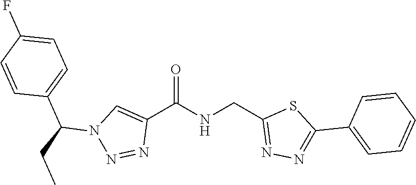 [SMILES: CC[C@@H](c1ccc(F)cc1)n1cc(C(=O)NCc2nnc(-c3ccccc3)s2)nn1] | (S)-1-(1-(4- fluorophenyl)propyl)-N-((5- phenyl-1,3,4-thiadiazol-2- yl)methyl)-1H-1,2,3- triazole-4-carboxamide | 423 |
| Example 258 | 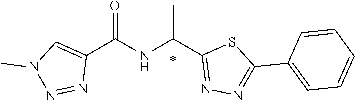 [SMILES: CC(NC(=O)c1cn(C)nn1)c1nnc(-c2ccccc2)s1] | 1-methyl-N-(1-(5-phenyl- 1,3,4-thiadiazol-2-yl)ethyl)- 1H-1,2,3-triazole-4- carboxamide, peak 1 | 315 |
| Example 259 | 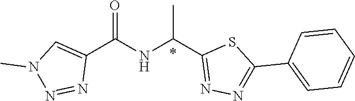 [SMILES: CC(NC(=O)c1cn(C)nn1)c1nnc(-c2ccccc2)s1] | 1-methyl-N-(1-(5-phenyl- 1,3,4-thiadiazol-2-yl)ethyl)- 1H-1,2,3-triazole-4- carboxamide, peak 2 | 315 |
| Example 260 | 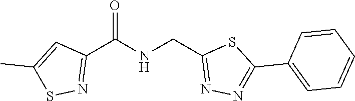 [SMILES: Cc1cc(C(=O)NCc2nnc(-c3ccccc3)s2)ns1] | 5-methyl-N-((5-phenyl- 1,3,4-thiadiazol-2- yl)methyl)isothiazole-3- carboxamide | 317 |
| Example 261 | 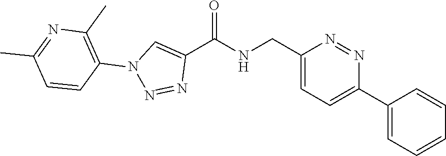 [SMILES: Cc1ccc(-n2cc(C(=O)NCc3ccc(-c4ccccc4)nn3)nn2)c(C)n1] | 1-(2,6-dimethylpyridin-3- yl)-N-((6-phenylpyridazin- 3-yl)methyl)-1H-1,2,3- triazole-4-carboxamide | 386 |
| Example 262 | 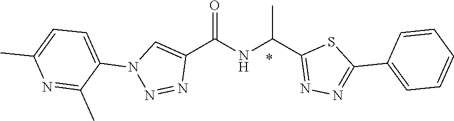 | 1-(2,6-dimethylpyridin-3- yl)-N-(1-(5-phenyl-1,3,4- thiadiazol-2-yl)ethyl)-1H- 1,2,3-triazole-4- carboxamide, peak 1 | 406 |
| Example 263 | 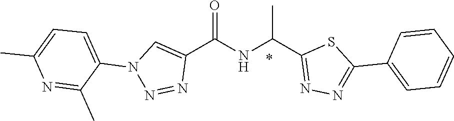 | 1-(2,6-dimethylpyridin-3- yl)-N-(1-(5-phenyl-1,3,4- thiadiazol-2-yl)ethyl)-1H- 1,2,3-triazole-4- carboxamide, peak 2 | 406 |
| Example 264 | 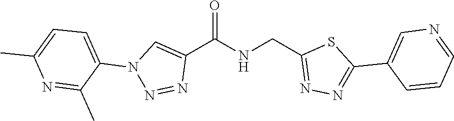 | 1-(2,6-dimethylpyridin-3- yl)-N-((5-(pyridin-3-yl)- 1,3,4-thiadiazol-2- yl)methyl)-1H-1,2,3- triazole-4-carboxamide | 393 |
| Example 265 | 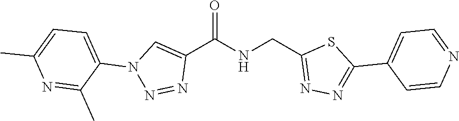 | 1-(2,6-dimethylpyridin-3- yl)-N-((5-(pyridin-4-yl)- 1,3,4-thiadiazol-2- yl)methyl)-1H-1,2,3- triazole-4-carboxamide | 393 |
| Example 266 | 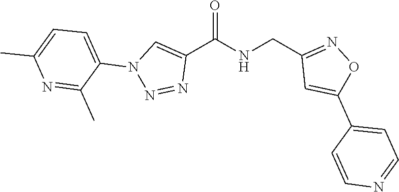 | 1-(2,6-dimethylpyridin-3- yl)-N-((5-(pyridin-4- yl)isoxazol-3-yl)methyl)- 1H-1,2,3-triazole-4- carboxamide | 376 |
| Example 267 | 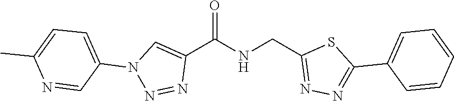 | 1-(6-methylpyridin-3-yl)-N- ((5-phenyl-1,3,4-thiadiazol- 2-yl)methyl)-1H-1,2,3- triazole-4-carboxamide | 378 |
| Example 268 | 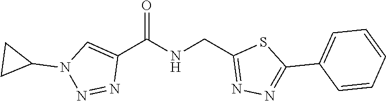 | 1-cyclopropyl-N-((5- phenyl-1,3,4-thiadiazol-2- yl)methyl)-1H-1,2,3- triazole-4-carboxamide | 327 |
| Example 269 | 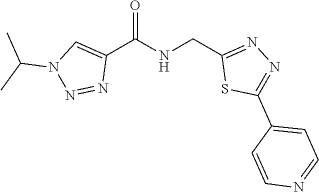 | 1-isopropyl-N-((5-(pyridin- 4-yl)-1,3,4-thiadiazol-2- yl)methyl)-1H-1,2,3- triazole-4-carboxamide | 330 |
| Example 270 | 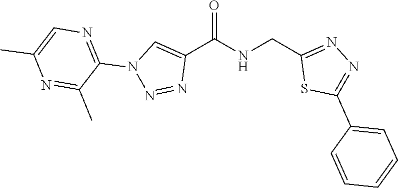 | 1-(3,5-dimethylpyrazin-2- yl)-N-((5-phenyl-1,3,4- thiadiazol-2-yl)methyl)-1H- 1,2,3-triazole-4- carboxamide | 393 |
| Example 271 | 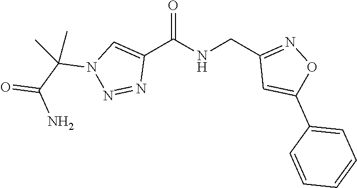 | 1-(1-amino-2-methyl-1- oxopropan-2-yl)-N-((5- phenylisoxazol-3- yl)methyl)-1H-1,2,3- triazole-4-carboxamide | 355 |
| Example 272 | 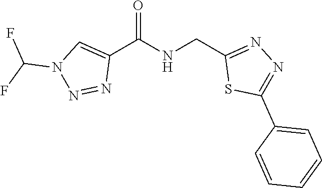 | 1-(difluoromethyl)-N-((5- phenyl-1,3,4-thiadiazol-2- yl)methyl)-1H-1,2,3- triazole-4-carboxamide | 337 |
| Example 273 | 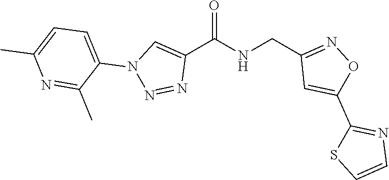 | 1-(2,6-dimethylpyridin-3- yl)-N-((5-(thiazol-2- yl)isoxazol-3-yl)methyl)- 1H-1,2,3-triazole-4- carboxamide | 382 |
| Example 274 | 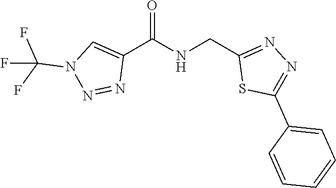 | N-((5-phenyl-1,3,4- thiadiazol-2-yl)methyl)-1- (trifluoromethyl)-1H-1,2,3- triazole-4-carboxamide | 355 |
| Example 275 | 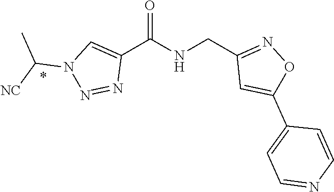 | 1-(1-cyanoethyl)-N-((5- (pyridin-4-yl)isoxazol-3- yl)methyl)-1H-1,2,3- triazole-4-carboxamide, peak 1 | 324 |
| Example 276 | 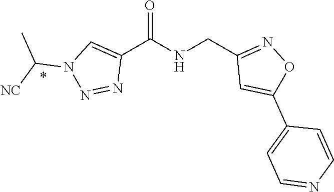 | 1-(1-cyanoethyl)-N-((5- (pyridin-4-yl)isoxazol-3- yl)methyl)-1H-1,2,3- triazole-4-carboxamide, peak 2 | 324 |
| Example 277 | 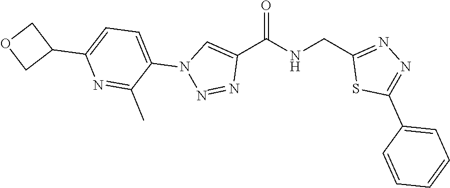 | 1-(2-methyl-6-(oxetan-3- yl)pyridin-3-yl)-N-((5- phenyl-1,3,4-thiadiazol-2- yl)methyl)-1H-1,2,3- triazole-4-carboxamide | 434 |
| Example 278 | 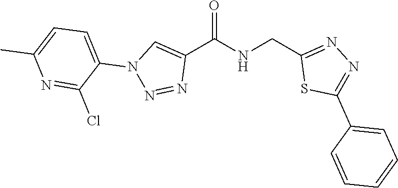 | 1-(2-chloro-6- methylpyridin-3-yl)-N-((5- phenyl-1,3,4-thiadiazol-2- yl)methyl)-1H-1,2,3- triazole-4-carboxamide | 412 |
| Example 279 | 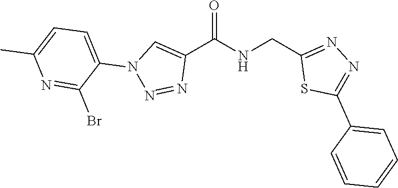 | 1-(2-bromo-6- methylpyridin-3-yl)-N-((5- phenyl-1,3,4-thiadiazol-2- yl)methyl)-1H-1,2,3- triazole-4-carboxamide | 456 |
| Example 280 | 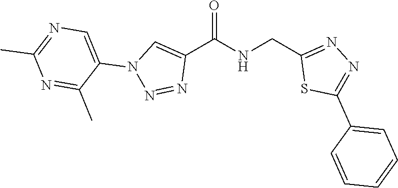 | 1-(2,4-dimethylpyrimidin- 5-yl)-N-((5-phenyl-1,3,4- thiadiazol-2-yl)methyl)-1H- 1,2,3-triazole-4- carboxamide | 393 |
| Example 281 | 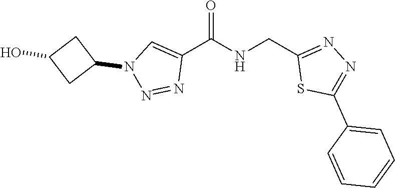 | 1-trans-3- hydroxycyclobutyl)-N-((5- phenyl-1,3,4-thiadiazol-2- yl)methyl)-1H-1,2,3- triazole-4-carboxamide | 357 |
| Example 282 | 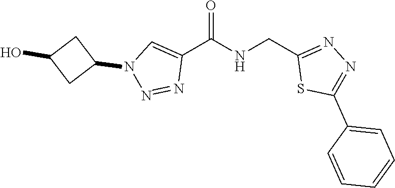 | 1-cis-3-hydroxycyclobutyl)- N-((5-phenyl-1,3,4- thiadiazol-2-yl)methyl)-1H- 1,2,3-triazole-4- carboxamide | 357 |
| Example 283 | 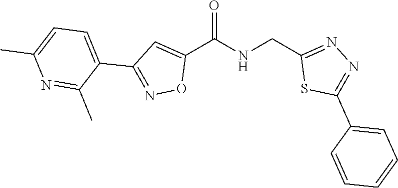 | 3-(2,6-dimethylpyridin-3- yl)-N-((5-phenyl-1,3,4- thiadiazol-2- yl)methyl)isoxazole-5- carboxamide | 392 |
| Example 284 | 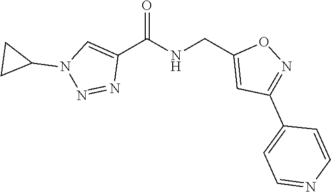 | 1-cyclopropyl-N-((3- (pyridin-4-yl)isoxazol-5- yl)methyl)-1H-1,2,3- triazole-4-carboxamide | 311 |
| Example 285 | 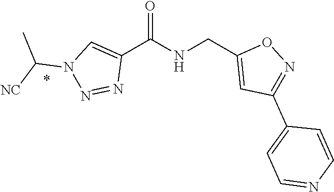 | 1-(1-cyanoethyl)-N-((3- (pyridin-4-yl)isoxazol-5- yl)methyl)-1H-1,2,3- triazole-4-carboxamide, peak 1 | 324 |
| Example 286 | 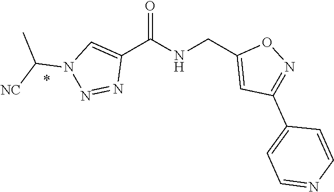 | 1-(1-cyanoethyl)-N-((3- (pyridin-4-yl)isoxazol-5- yl)methyl)-1H-1,2,3- triazole-4-carboxamide, peak 2 | 324 |
| Example 287 | 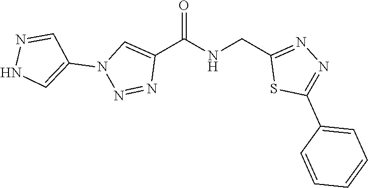 | N-((5-phenyl-1,3,4- thiadiazol-2-yl)methyl)-1- (1H-pyrazol-4-yl)-1H- 1,2,3-triazole-4- carboxamide | 353 |
| Example 288 | 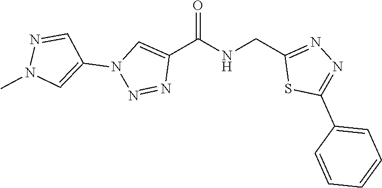 | 1-(1-methyl-1H-pyrazol-4- yl)-N-((5-phenyl-1,3,4- thiadiazol-2-yl)methyl)-1H- 1,2,3-triazole-4- carboxamide | 367 |
| Example 289 | 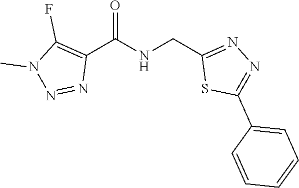 | 5-fluoro-1-methyl-N-((5- phenyl-1,3,4-thiadiazol-2- yl)methyl)-1H-1,2,3- triazole-4-carboxamide | 319 |

### Separation of Racemic 1-cyclopropyl-N-(1-(5-phenyl-1,3,4-thiadiazol-2-yl)ethyl)-1H-1,2,3-triazole-4-carboxamide, Example 244 and 245

The racemic mixture of 1-cyclopropyl-N-(1-(5-phenyl-1,3,4-thiadiazol-2-yl)ethyl)-1H-1,2,3-triazole-4-carboxamide was resolved by chiral SFC purification (Daicel Chiralcel OJ 21×250 mm column, 40% EtOH w/0.1% NH₄OH as cosolvent).

### Separation of Racemic 1-(2,2-difluoroethyl)-N-(1-(5-phenyl-1,3,4-thiadiazol-2-yl)ethyl)-1H-1,2,3-triazole-4-carboxamide, Examples 251 and 252

The racemic mixture of 1-(2,2-difluoroethyl)-N-(1-(5-phenyl-1,3,4-thiadiazol-2-yl)ethyl)-1H-1,2,3-triazole-4-carboxamide was resolved by chiral SFC purification (Chiralpak IC-3 150×4.6 mm column, 40% EtOH w/0.05% DEA as cosolvent).

### Separation of Racemic 1-(2,6-dimethylpyridin-3-yl)-N-(1-(5-phenyl-1,3,4-thiadiazol-2-yl)ethyl)-1H-1,2,3-triazole-4-carboxamide, Example 262 and 263

The racemic mixture of 1-(2,6-dimethylpyridin-3-yl)-N-(1-(5-phenyl-1,3,4-thiadiazol-2-yl)ethyl)-1H-1,2,3-triazole-4-carboxamide was resolved by Chiral SFC purification (Chiralcel OJ-3 100×4.6 mm, 40% EtOH w/0.05% DEA as cosolvent).

### Separation of Racemic 1-(1-cyanoethyl)-N-((5-(pyridin-4-yl)isoxazol-3-yl)methyl)-1H-1,2,3-triazole-4-carboxamide, Example 275 and 276

The racemic mixture of 1-(1-cyanoethyl)-N-((5-(pyridin-4-yl)isoxazol-3-yl)methyl)-1H-1,2,3-triazole-4-carboxamide was resolved by chiral SFC purification (Daicel Chiralpak AD 250×30 mm column, 35% IPA w/0.05% DEA as cosolvent).

### Example 290

### Preparation of 1-(oxetan-3-yl)-N-((3-phenylisoxazol-5-yl)methyl)-1H-1,2,3-triazole-4-carboxamide

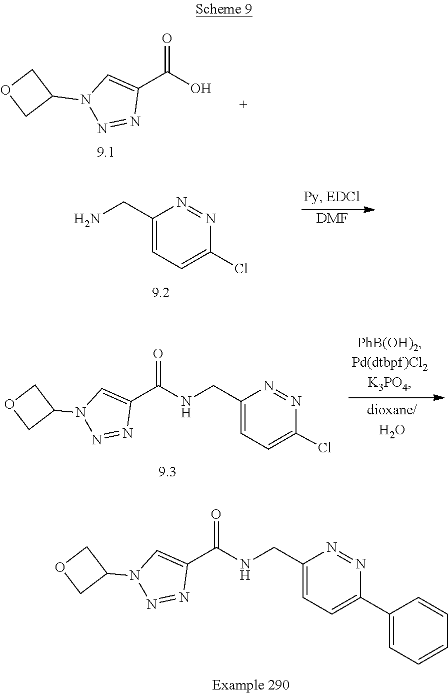

### Step 1. Preparation of N-((6-(chloromethyl)pyridazin-3-yl)methyl)-1-(oxetan-3-yl)-1H-1,2,3-triazole-4-carboxamide

To a solution of 1-(oxetan-3-yl)-1H-1,2,3-triazole-4-carboxylic acid (90 mg, 0.532 mmol) in DMF (3 mL) was added pyridine (126 mg, 1.596 mmol) and 3-(((ethylimino)methylene)amino)-N,N-dimethylpropan-1-amine hydrochloride (153 mg, 0.798 mmol) at 25° C. over 1 minute. After stirring for 2 minutes at 25° C., (6-(chloromethyl)pyridazin-3-yl)methanamine (84 mg, 0.532 mmol) was added to the mixture at 25° C. The resulting mixture was stirred for another 2 hours. The mixture was filtered and the filtrate was purified via reverse phase HPLC (eluting MeCN in water with 0.1% TFA modifier) to afford N-((6-(chloromethyl)pyridazin-3-yl)methyl)-1-(oxetan-3-yl)-1H-1,2,3-triazole-4-carboxamide. LC/MS (ESI) m/z: 295 (M+H)⁺.

### Step 2. 1-(oxetan-3-yl)-N-((6-phenylpyridazin-3-yl)methyl)-1H-1,2,3-triazole-4-carboxamide

A mixture of N-((6-chloropyridazin-3-yl)methyl)-1-(oxetan-3-yl)-1H-1,2,3-triazole-4-carboxamide (30 mg, 0.102 mmol), phenylboronic acid (18.62 mg, 0.153 mmol), potassium phosphate tribasic (64.8 mg, 0.305 mmol) and PdCl₂(dtbpf) (6.63 mg, 10.18 μmol) in dioxane:H₂O=5:1 (3 mL) was degassed and backfilled with N₂ (three times). The mixture was heated to 80° C. for 2 hours. After cooling to room temperature the mixture was filtered and the filtrate was purified via reverse phase HPLC (eluting MeCN in water with 10 mM NH₄HCO₃) to afford 1-(oxetan-3-yl)-N-((6-phenylpyridazin-3-yl)methyl)-1H-1,2,3-triazole-4-carboxamide). LC/MS (ESI) m/z: 337 (M+H)⁺. ¹H NMR (400 MHz, CDCl₃) δ 8.39 (s, 1H), 8.11-8.18 (m, 1H), 8.04-8.10 (m, 2H), 7.86 (d, J=8.82 Hz, 1H), 7.63 (d, J=8.82 Hz, 1H), 7.48-7.57 (m, 3H), 5.74-5.87 (m, 1H), 5.19 (t, J=7.45 Hz, 2H), 4.99-5.06 (m, 4H)

### Example 291

### Preparation of 1-(2,2-difluoroethyl)-N-((3-phenylisoxazol-5-yl)methyl)-1H-1,2,3-triazole-4-carboxamide

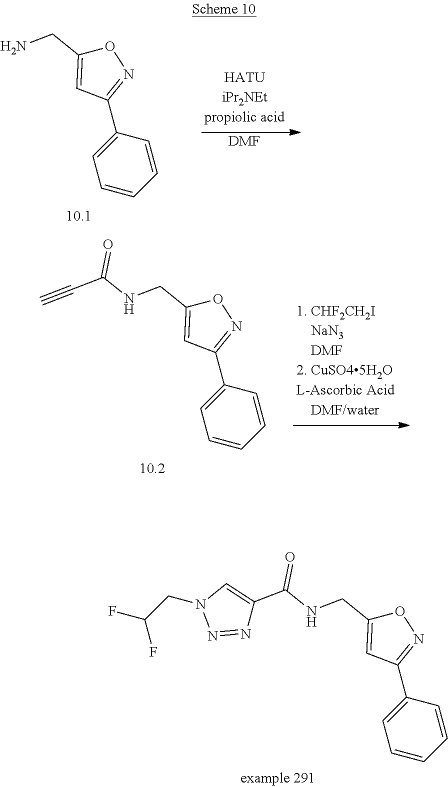

### Step 1. Preparation of N-((3-phenylisoxazol-5-yl)methyl)propiolamide

Propiolic acid (97 μL, 1.579 mmol) and (3-phenylisoxazol-5-yl)methanamine (250 mg, 1.435 mmol) were charged in a 20 mL round bottomed flask, DMF (4784 μL) and Hunig's Base (1303 μL, 7.46 mmol) were added followed by HATU (655 mg, 1.722 mmol) and the reaction was stirred overnight. The reaction was concentrated and the crude material was taken up in DCM and water, and the organic layer was separated using a phase separator. The organic layer was concentrated and the residue was purified by column chromatography on silica gel (eluting EtOAc in hexanes) to afford N-((3-phenylisoxazol-5-yl)methyl)propiolamide.

### Step 2. Preparation of 1-(2,2-difluoroethyl)-N-((3-phenylisoxazol-5-yl)methyl)-1H-1,2,3-triazole-4-carboxamide

Sodium azide (35.9 mg, 0.553 mmol) was added to a solution of 1,1-difluoro-2-iodoethane (48.6 μL, 0.553 mmol) in DMF (1105 μL) and the reaction was stirred at 60° C. for 24 hours. Then in the same pot a solution of N-((3-phenylisoxazol-5-yl)methyl)propiolamide (50 mg, 0.221 mmol) in DMF (1105 μL) was added followed by an aqueous solutions of copper(II) sulfate pentahydrate (5.52 mg, 0.022 mmol) and sodium ascorbate (8.76 mg, 0.044 mmol) was added and the reaction was stirred at room temperature overnight. The solution was filtered and purified via reverse phase HPLC (eluting MeCN in water gradient with ammonium hydroxide as a modifier) to afford 1-(2,2-difluoroethyl)-N-((3-phenylisoxazol-5-yl)methyl)-1H-1,2,3-triazole-4-carboxamide LC/MS (m/z): 334 (M+H)⁺. ¹H NMR (499 MHz, DMSO) δ 9.3 (s, 1H), 8.7 (s, 1H), 7.9 (d, J=3.0 Hz, 1H), 7.5 (s, 2H), 6.9 (s, 1H), 6.7-6.4 (m, 2H), 5.0 (t, J=15.0 Hz, 2H), 4.6 (d, J=5.9 Hz, 2H).

### Example 292

### Preparation of 1-(2,2-difluoroethyl)-N-((5-phenylisoxazol-3-yl)methyl)-1H-1,2,3-triazole-4-carboxamide

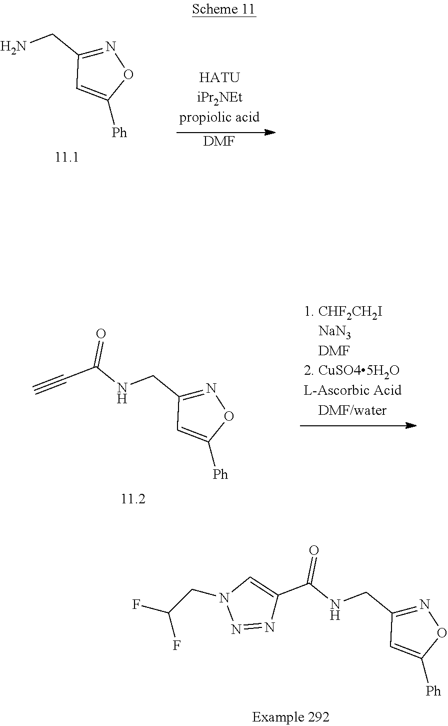

### Step 1. Preparation of N-((5-phenylisoxazol-3-yl)methyl)propiolamide

Propiolic acid (97 μL, 1.579 mmol) and (5-phenylisoxazol-3-yl)methanamine (250 mg, 1.435 mmol) were charged in a 20 mL round bottomed flask, DMF (4784 μL) and Hunig's base (1303 μL, 7.46 mmol) were added followed by HATU (655 mg, 1.72 mmol) and the reaction was stirred overnight. The reaction was concentrated and the residue was purified by column chromatography on silica gel (eluting EtOAc in hexanes) to afford N-((5-phenylisoxazol-3-yl)methyl)propiolamide.

### Step 2. Preparation of 1-(2,2-difluoroethyl)-N-((5-phenylisoxazol-3-yl)methyl)-1H-1,2,3-triazole-4-carboxamide

Sodium azide (35.9 mg, 0.553 mmol) as added to a solution of 2-bromo-1,1-difluoroethane (80 μL, 0.553 mmol) in DMF (1105 μL) and the reaction was stirred at 60° C. for 24 h. Then in the same pot a solution of N-((5-phenylisoxazol-3-yl)methyl)propiolamide (50 mg, 0.221 mmol) in DMF (1105 μL) was added followed by an aqueous solutions of copper(II) sulfate pentahydrate (5.52 mg, 0.022 mmol) and sodium ascorbate (8.76 mg, 0.044 mmol). The reaction was stirred at room temperature overnight. The reaction was filtered and purified via reverse phase HPLC (eluting MeCN in water gradient with ammonium hydroxide as a modifier) to afford 1-(2,2-difluoroethyl)-N-((5-phenylisoxazol-3-yl)methyl)-1H-1,2,3-triazole-4-carboxamide. LC/MS (m/z): 356 (M+Na)+. ¹H NMR (499 MHz, DMSO) δ 9.2 (s, 1H), 8.7 (s, 1H), 7.9 (d, J=6.7 Hz, 1H), 7.5 (d, J=7.7 Hz, 2H), 6.9 (s, 1H), 6.5 (t, J=54.2 Hz, 2H), 5.0 (t, J=15.4 Hz, 2H), 4.6 (d, J=5.9 Hz, 2H).

### Example 293

### Preparation of 1-(2,2-difluoroethyl)-N-((6-phenylpyridazin-3-yl)methyl)-1H-1,2,3-triazole-4-carboxamide

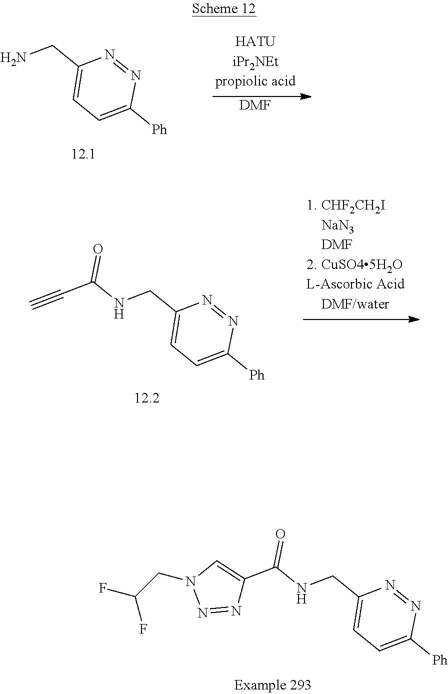

### Step 1: Preparation of N-((6-phenylpyridazin-3-yl)methyl)propiolamide

HATU (266 mg, 0.700 mmol) and Hunig's base (530 μL, 3.03 mmol) were added to a solution of propiolic acid (39.5 μL, 0.641 mmol) and (6-phenylpyridazin-3-yl)methanamine (108 mg, 0.583 mmol) in DMF (1944 μL). The reaction was allowed to stir for 16 hours and then the reaction was filtered, and concentrated. The residue was purified by column chromatography on silica gel (eluting with EtOAc in hexanes) to afford N-((6-phenylpyridazin-3-yl)methyl)propiolamide.

### Step 2: Preparation of 1-(2,2-difluoroethyl)-N-((6-phenylpyridazin-3-yl)methyl)-1H-1,2,3-triazole-4-carboxamide

Sodium azide (34.3 mg, 0.527 mmol) was added to a solution of 1,1-difluoro-2-iodoethane (46.4 μL, 0.527 mmol) in DMF (1054 μL) and the reaction was stirred at 60° C. for 24 hours. In the same pot a solution of N-((6-phenylpyridazin-3-yl)methyl)propiolamide (50 mg, 0.211 mmol) in DMF (1054 μL) was added followed by an aqueous solutions of copper(II) sulfate pentahydrate (5.26 mg, 0.021 mmol) and sodium ascorbate (8.35 mg, 0.042 mmol). The reaction was stirred at room temperature overnight. 3-Mercaptopropyl-functionalized silica gel was added to the reaction to scavenge the copper, and then the reaction was filtered and purified via reverse phase HPLC (eluting MeCN in water with 0.1% TFA modifier) to afford 1-(2,2-difluoroethyl)-N-((6-phenylpyridazin-3-yl)methyl)-1H-1,2,3-triazole-4-carboxamide. LC/MS (m/z): 345 (M+H)⁺. ¹H NMR (499 MHz, DMSO) δ 9.4 (s, 1H), 8.7 (s, 1H), 8.2 (d, J=8.5 Hz, 1H), 8.1 (d, J=6.9 Hz, 2H), 7.7 (d, J=8.7 Hz, 1H), 7.6 (d, J=7.2 Hz, 2H), 6.7-6.4 (m, 2H), 5.1-5.0 (m, 1H), 4.8 (d, J=5.8 Hz, 2H).

### Example 294

### Preparation of 1-methyl-N-((5-(piperidin-1-yl)-1,3,4-thiadiazol-2-yl)methyl)-1H-1,2,3-triazole-4-carboxamide

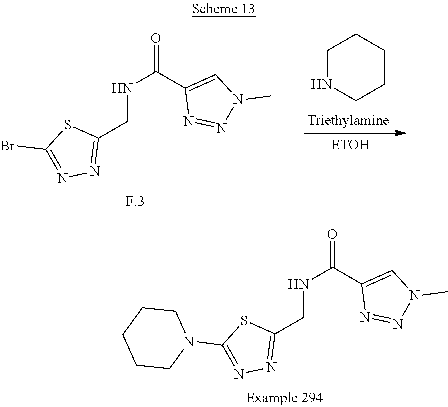 [SMILES: Cn1cc(C(=O)NCc2nnc(Br)s2)nn1 ; C1CCNCC1 ; Cn1cc(C(=O)NCc2nnc(N3CCCCC3)s2)nn1]

In a 2 mL microwave vial containing N-((5-bromo-1,3,4-thiadiazol-2-yl)methyl)-1-methyl-1H-1,2,3-triazole-4-carboxamide (40 mg, 0.13 mmol) and piperidine (131 μL, 1.32 mmol) in ethanol (0.7 mL) was added triethylamine (55 μL, 0.40 mmol). The reaction was heated in a microwave reactor at 120° C. for 10 minutes. The reaction was cooled to room temperature and the residue was purified via reverse phase HPLC (eluting acetonitrile in water, with TFA modifier) to afford 1-methyl-N-((5-(piperidin-1-yl)-1,3,4-thiadiazol-2-yl)methyl)-1H-1,2,3-triazole-4-carboxamide. LC/MS (m/z): 308 (M+H)⁺. ¹H NMR (600 MHz, DMSO-d₆) δ 9.32 (t, J=6.0 Hz, 1H), 8.56 (s, 1H), 4.59 (d, J=6.1 Hz, 2H), 4.10 (s, 3H), 3.60 (s, 65H), 3.41 (s, 4H), 1.58 (s, 6H).

### Example 295

### Preparation of N-[[5-(4-hydroxyphenyl)-1,3,4-thiadiazol-2-yl]methyl]-1-methyl-triazole-4-carboxamide

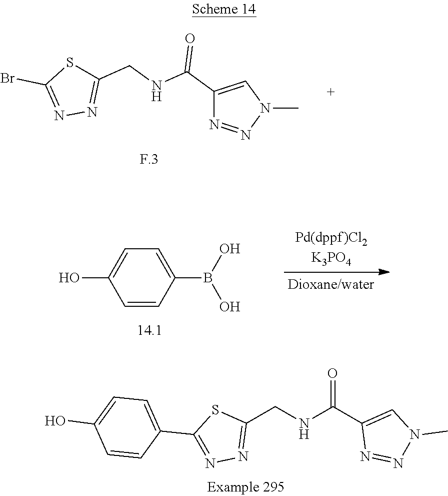 [SMILES: Cn1cc(C(=O)NCc2nnc(Br)s2)nn1 ; OB(O)c1ccc(O)cc1 ; Cn1cc(C(=O)NCc2nnc(-c3ccc(O)cc3)s2)nn1]

Pd(dppf)Cl₂ (9 mg, 0.012 mmol) in 10:1 water/dioxane (0.5 mL) was added to a microwave vial containing (4-hydroxyphenyl)boronic acid (22 mg, 0.16 mmol), N-((5-bromo-1,3,4-thiadiazol-2-yl)methyl)-1-methyl-1H-1,2,3-triazole-4-carboxamide (24 mg, 0.08 mmol) and K₃PO₄ (52 mg, 0.24 mmol). The vial was purged with argon for 5 minutes then it was heated in to 120° C. for 30 minutes in a microwave reactor. The reaction was cooled to room temperature, diluted with MeOH and purified via reverse phase HPLC (eluting acetonitrile in water, with TFA modifier) to afford N-((5-(4-hydroxyphenyl)-1,3,4-thiadiazol-2-yl)methyl)-1-methyl-1H-1,2,3-triazole-4-carboxamide. LC/MS (m/z): 317 (M+H)⁺.

Examples shown in Example Table 9 below, were prepared according to procedures analogous to those outlined in Example 295 and General Scheme 4 above using the appropriate starting materials obtained from methods described above or elsewhere, or as obtained from commercial sources.

Example Table 9

| | | | Mass |
|---|---|---|---|
| Example | Structure | Name | [M + H]⁺ |
| Example 296 | 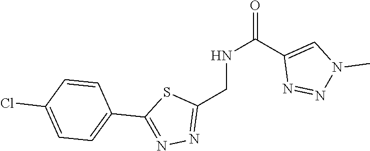 [SMILES: Cn1cc(C(=O)NCc2nnc(-c3ccc(Cl)cc3)s2)nn1] | N-((5-(4-chlorophenyl)- 1,3,4-thiadiazol-2- yl)methyl)-1-methyl- 1H-1,2,3-triazole-4- carboxamide | 335 |
| Example 297 | 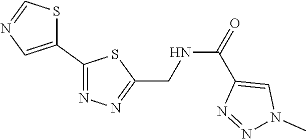 [SMILES: Cn1cc(C(=O)NCc2nnc(-c3cncs3)s2)nn1] | 1-methyl-N-((5-(thiazol-5- yl)-1,3,4-thiadiazol-2- yl)methyl)-1H-1,2,3- triazole-4-carboxamide | 308 |
| Example 298 | 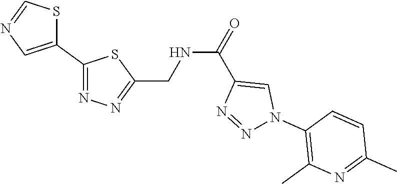 [SMILES: Cc1ccc(-n2cc(C(=O)NCc3nnc(-c4cncs4)s3)nn2)c(C)n1] | 1-(2,6-dimethylpyridin-3- yl)-N-((5-(thiazol- 5-yl)-1,3,4-thiadiazol-2- yl)methyl)-1H-1,2,3- triazole-4-carboxamide | 399 |

### Example 299

### Preparation of N-((5-(1H-pyrazol-4-yl)-1,3,4-thiadiazol-2-yl)methyl)-1-methyl-1H-1,2,3-triazole-4-carboxamide

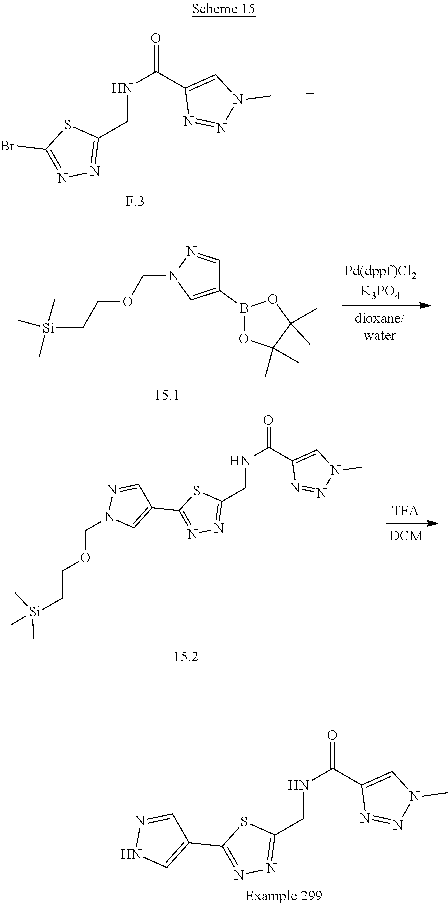

### Step 1. Preparation of 1-methyl-N-((5-(1-((2-(trimethylsilyl)ethoxy)methyl)-1H-pyrazol-4-yl)-1,3,4-thiadiazol-2-yl)methyl)-1H-1,2,3-triazole-4-carboxamide

A 2 mL microwave vial containing 4-(4,4,5,5-tetramethyl-1,3,2-dioxaborolan-2-yl)-1-((2-(trimethylsilyl)ethoxy)methyl)-1H-pyrazole (72.2 mg, 0.223 mmol) and N-((5-bromo-1,3,4-thiadiazol-2-yl)methyl)-1-methyl-1H-1,2,3-triazole-4-carboxamide (25 mg, 0.082 mmol) in 1,4-dioxane (500 μL)/water (50.0 μL) was purged with Ar_{(g)} and maintained under inert atmosphere. PdC₂(dppf) (9.05 mg, 0.012 mmol) and potassium phosphate tribasic (52.5 mg, 0.247 mmol) were added and the reaction was heated in a microwave reactor at a 120° C. for 15 minutes. The reaction mixture was cooled to room temperature, diluted with MeOH and filtered through a SiliaMetS® ((Si-thiol) (1 g). The filtrate was concentrated to afford 1-methyl-N-((5-(1-((2-(trimethylsilyl)ethoxy)methyl)-1H-pyrazol-4-yl)-1,3,4-thiadiazol-2-yl)methyl)-1H-1,2,3-triazole-4-carboxamide 15.2. The crude material was dissolved in DCM (1 mL) was added TFA (159 μL, 2.1 mmol). The reaction was stirred at room temperature overnight. The reaction was concentrated and purified via reverse phase HPLC (eluting acetonitrile in water, with NH₄OH modifier) to afford N-((5-(1H-pyrazol-4-yl)-1,3,4-thiadiazol-2-yl)methyl)-1-methyl-1H-1,2,3-triazole-4-carboxamide. LC/MS (m/z): 291 (M+H)⁺. ¹H NMR (600 MHz, DMSO-d₆) δ 13.41 (s, 1H), 9.50 (t, J=5.6 Hz, 1H), 8.59 (s, 1H), 8.46 (s, 1H), 8.03 (s, 1H), 4.81 (d, J=5.9 Hz, 2H), 4.11 (s, 3H).

Examples shown in Example Table 10 below, were according to procedures analogous to those outlined in Example 299 and General Scheme 4 above using the appropriate starting materials obtained from methods described above or elsewhere, or as obtained from commercial sources.

Example Table 10

| | | | Mass |
|---|---|---|---|
| Example | Structure | Name | [M + H]⁺ |
| Example 300 | 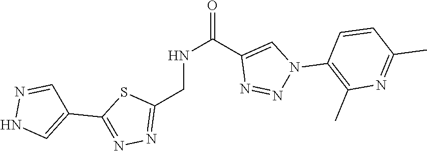 [SMILES: Cc1ccc(-n2cc(C(=O)NCc3nnc(-c4cn[nH]c4)s3)nn2)c(C)n1] | N-((5-(1H-pyrazol-4-yl)- 1,3,4-thiadiazol-2- yl)methyl)-1-(2,6- dimethylpyridin-3-yl)-1H- 1,2,3-triazole-4- carboxamide | 382 |

### Example 301

### Preparation of 1-(6-(difluoromethoxy)-2-methylpyridin-3-yl)-N-((5-phenyl-1,3,4-thiadiazol-2-yl)methyl)-1H-1,2,3-triazole-4-carboxamide

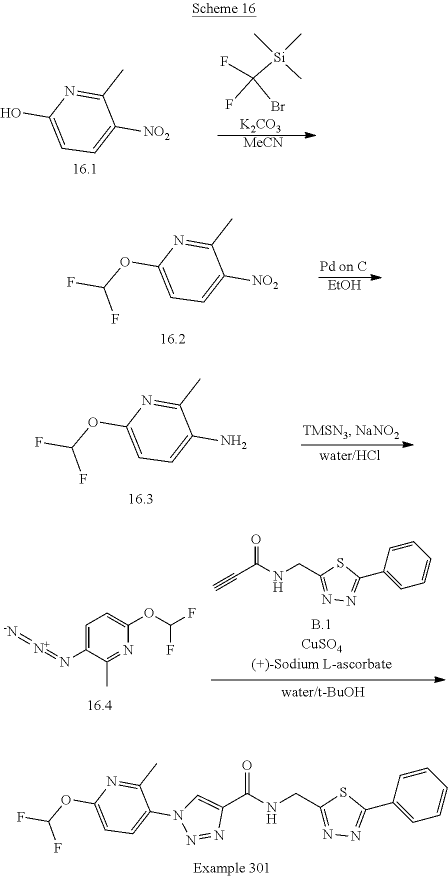

### Step 1. Preparation of 6-(difluoromethoxy)-2-methyl-3-nitropyridine

6-methyl-5-nitropyridin-2-ol (250 mg, 1.622 mmol) and potassium carbonate (336 mg, 2.433 mmol) in acetonitrile (6 mL) were added to a 20 mL vial. The suspension was stirred and heated at 60° C. for 10 minutes. Then (bromodifluoromethyl)trimethyl silane (377 μL, 2.43 mmol) was added. The reaction mixture was stirred at 60° C. for 1 hour. The reaction was quenched with water (5 mL) and extracted with DCM (3×5 mL). The organic extract was concentrated to afford 6-(difluoromethoxy)-2-methyl-3-nitropyridine that was used directly in the next step.

### Step 2. Preparation of 6-(difluoromethoxy)-2-methylpyridin-3-amine

Palladium on carbon (173 mg, 0.162 mmol) was added to a 40 mL vial containing 6-(difluoromethoxy)-2-methyl-3-nitropyridine. The vessel was purged with N_{2(g)} and maintained under an inert atmosphere. Then EtOH (6 mL) was added and the reaction was stirred at room temperature for 3 days under an atmosphere of H₂ (1 atm). The reaction mixture was filtered through celite plug and the filtrate was concentrated to afford 6-(difluoromethoxy)-2-methylpyridin-3-amine which used directly in the next step. LC/MS (m/z): 175 (M+H)⁺.

### Step 3. Preparation of 3-azido-6-(difluoromethoxy)-2-methylpyridine

Sodium nitrite (107 mg, 1.550 mmol) in water (0.65 mL) was added to a solution of 6-(difluoromethoxy)-2-methylpyridin-3-amine (180 mg, 1.03 mmol) in aq. HCl (6M) (1.9 mL) at 0° C. over 2 minutes. After stirring for 15 minutes at 0° C., TMS-N₃ (274 μL, 2.067 mmol) was added to the mixture. The resulting mixture was warmed to room temperature and stirred for 0.5 hours. The mixture was cooled to 0° C. and made basic (pH >9) with aqueous NaOH (1N) and extracted with EtOAc (3×5 mL). The combined organic phases were washed with brine (3×5 mL), dried over anhydrous MgSO₄, filtered and concentrated under reduced pressure. The residue was purified by column chromatography on silica gel (eluting EtOAc in Hexanes) to afford 3-azido-6-(difluoromethoxy)-2-methylpyridine. LC/MS (m/z): 201 (M+H)⁺.

### Step 4. Preparation of 1-(6-(difluoromethoxy)-2-methylpyridin-3-yl)-N-((5-phenyl-1,3,4-thiadiazol-2-1)methyl)-1H-1,2,3-triazole-4-carboxamide

N-((5-phenyl-1,3,4-thiadiazol-2-yl)methyl)propiolamide (66.9 mg, 0.275 mmol) was added to a solution containing 3-azido-6-(difluoromethoxy)-2-methylpyridine (50 mg, 0.250 mmol), (+)-sodium L-ascorbate (9.90 mg, 0.050 mmol) and copper (II) sulfate (3.99 mg, 0.025 mmol) in t-BuOH (500 μL) and water (500 μL) at room temperature. The mixture was heated at 50° C. for 0.5 hours. The reaction was quenched with water (2 mL) and extracted with DCM (3×5 mL). The organic extract was concentrated and purified via reverse phase HPLC (eluting acetonitrile in water, with NH₄OH modifier) to afford 1-(6-(difluoromethoxy)-2-methylpyridin-3-yl)-N-((5-phenyl-1,3,4-thiadiazol-2-yl)methyl)-1H-1,2,3-triazole-4-carboxamide. LC/MS (m/z): 444 (M+H)⁺. ¹H NMR (600 MHz, DMSO-d₆) δ 9.74 (t, J=6.0 Hz, 1H), 9.12 (s, 1H), 8.13 (d, J=8.6 Hz, 1H), 8.02-7.96 (m, 2H), 7.81 (t, J=72.4 Hz, 1H), 7.56 (q, J=6.7, 6.1 Hz, 3H), 7.20 (d, J=8.6 Hz, 1H), 4.93 (d, J=6.1 Hz, 2H), 2.34 (s, 3H).

### Example 302

### Preparation of (S)-1-(2-fluoro-3-hydroxypropyl)-N-((5-phenyl-1,3,4-thiadiazol-2-yl)methyl)-1H-1,2,3-triazole-4-carboxamide

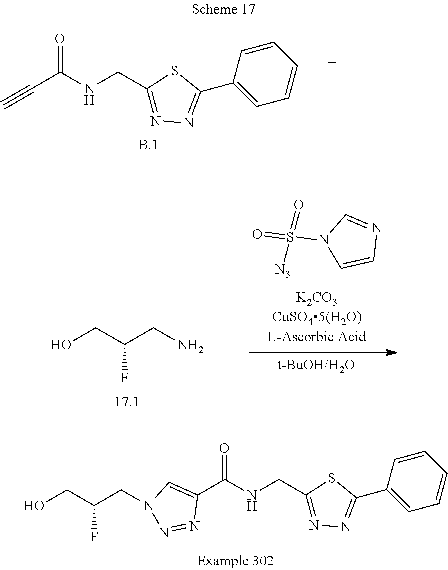 [SMILES: C#CC(=O)NCc1nnc(-c2ccccc2)s1 ; [N-]=[N+]=NS(=O)(=O)n1ccnc1 ; NC[C@H](F)CO ; O=C(NCc1nnc(-c2ccccc2)s1)c1cn(C[C@H](F)CO)nn1]

tert-butanol (27 mL) was added to a 100-mL round bottom flask containing potassium carbonate (4.0 g, 29 mmol) copper (II) sulfate pentahydrate (0.62 g, 2.5 mmol), and (S)-3-amino-2-fluoropropan-1-ol (0.50 g, 5.3 mmol). 1H-imidazole-1-sulfonyl azide hydrochloride (1.1 g, 5.3 mmol) was added, followed by water (14 mL). The reaction was stirred for 30 minutes then L-ascorbic acid (1.3 g, 6.6 mmol) and N-((5-phenyl-1,3,4-thiadiazol-2-yl)methyl)propiolamide (1.0 g, 4.1 mmol) were added. The reaction was stirred for one hour, the mixture was diluted with ethyl acetate (50 mL) and saturated sodium bicarbonate (50 mL). The resulting mixture was filtered through CELITE and the CELITE was washed with ethyl acetate (2×20 mL). The organic layer of the filtrate was then separated, dried over magnesium sulfate and filtered. Silica gel (15 g) was added to the filtrate and the mixture was concentrated under reduced pressure and placed under vacuum. The crude material was purified by silica gel column chromatography (eluting EtOAc in hexanes). Dichloromethane (˜100 mL) and water (˜100 mL) were added to the isolated residue and the mixture was stirred. The solids were filtered, washed with diethyl ether (2×50 mL) and dried under vacuum overnight to afford (S)-1-(2-fluoro-3-hydroxypropyl)-N-((5-phenyl-1,3,4-thiadiazol-2-yl)methyl)-1H-1,2,3-triazole-4-carboxamide. LC/MS (m/z): 363 (M+H)⁺. 1H NMR (500 MHz, DMSO-d6) δ 9.59 (t, J=5.9 Hz, 1H), 8.69 (s, 1H), 7.96 (d, J=7.5 Hz, 2H), 7.59-7.51 (m, 3H), 5.25 (t, J=5.4 Hz, 1H), 5.04-4.90 (m, 1H), 4.88 (d, J=5.9 Hz, 2H), 4.80-4.77 (m, 1H), 4.76-4.73 (m, 1H), 3.76-3.54 (m, 2H).

### Example 303

### Preparation of (R)-1-(1-hydroxypropan-2-yl)-N-((5-phenyl-1,3,4-thiadiazol-2-yl)methyl)-1H-1,2,3-triazole-4-carboxamide

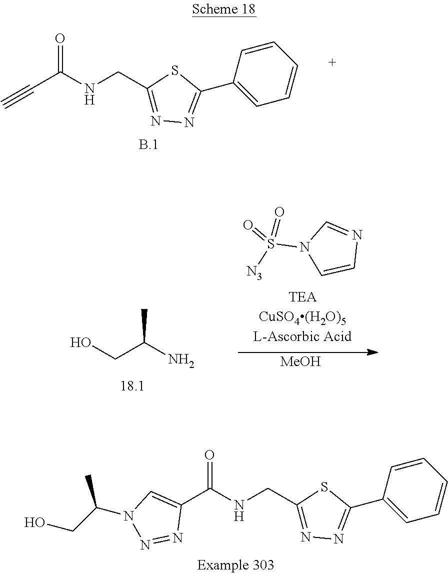

N-((5-phenyl-1,3,4-thiadiazol-2-yl)methyl)propiolamide (97 mg, 0.34 mmol), (R)-2-aminopropan-1-ol (30 mg, 0.40 mmol), 1H-imidazole-1-sulfonyl azide (84 mg, 0.79 mmol), copper (II) sulfate pentahydrate (10 mg, 0.040 mmol), and L-ascorbic acid (16 mg, 0.080 mmol) were charged in a 2 dram vial with stir bar and septa and the vial was evacuated and backfilled with argon (3×). MeOH (2.0 mL) was added, followed by triethylamine (110 μL) and the reaction was stirred at room temperature for 16 hours. The resulting mixture was diluted with EtOAc (50 mL) and washed with saturated NaHCO₃ (1×50 mL) and water (3×50 mL). The organic layers were combined and washed with brine (50 mL), dried over anhydrous MgSO₄, and concentrated under reduced pressure. The solid was purified via reverse phase HPLC (eluting acetonitrile in water with a 0.1% TFA modifier) to afford (R)-1-(1-hydroxypropan-2-yl)-N-((5-phenyl-1,3,4-thiadiazol-2-yl)methyl)-1H-1,2,3-triazole-4-carboxamide. LC/MS (m/z): 345 (M+H)⁺. ¹H NMR (499 MHz, DMSO) δ 9.53 (s, 1H), 8.66 (s, 1H), 7.99-7.92 (m, 2H), 7.58-7.50 (m, 2H), 5.11 (s, 1H), 4.87 (d, J=5.0 Hz, 2H), 4.80-4.72 (m, 1H), 3.75-3.69 (m, 1H), 1.47 (d, J=6.8 Hz, 3H).

### Example 304

### Preparation of 1-isopropyl-N-((5-phenyl-1,3,4-thiadiazol-2-yl)methyl)-1H-1,2,3-triazole-4-carboxamide

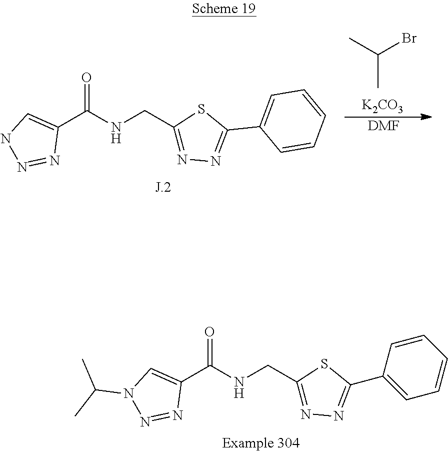

A mixture of N-((5-phenyl-1,3,4-thiadiazol-2-yl)methyl)-1H-1,2,3-triazole-4-carboxamide (50 mg, 0.175 mmol), K₂CO₃ (72.4 mg, 0.524 mmol) and 2-bromopropane (107 mg, 0.873 mmol) in DMF (1 mL) was stirred at 50° C. for 2 hours. The reaction mixture was filtered and the residue was purified via reverse phase HPLC (eluting acetonitrile in water with 0.1% TFA) to afford 1-isopropyl-N-((5-phenyl-1,3,4-thiadiazol-2-yl)methyl)-1H-1,2,3-triazole-4-carboxamide. LC/MS (ESI) m/z: 329 (M+H)⁺. ¹H NMR (400 MHz, MeOD) δ 8.46 (s, 1H), 7.94 (dd, J=1.73, 7.81 Hz, 2H), 7.48-7.57 (m, 3H), 5.00 (s, 2H), 4.63 (br s, 1H), 1.60 (d, J=6.68 Hz, 6H).

Examples shown in Example Table 11 below, were prepared according to procedures analogous to those outlined in Example 304 and General Scheme 5 above using the appropriate starting materials obtained from methods described above or elsewhere, or as obtained from commercial sources.

Example Table 11

| | | | Mass |
|---|---|---|---|
| Example | Structure | Name | [M + H]⁺ |
| Example 305 | 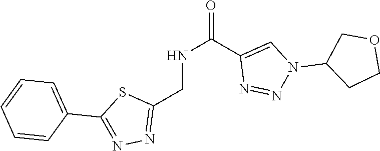 [SMILES: O=C(NCc1nnc(-c2ccccc2)s1)c1cn(C2CCOC2)nn1] | N-((5-phenyl-1,3,4- thiadiazol-2-yl)methyl)-1- (tetrahydrofuran-3-yl)-1H- 1,2,3-triazole-4- carboxamide | 357 |
| Example 306 | 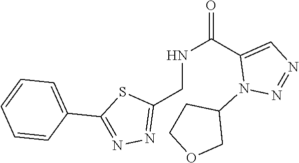 [SMILES: O=C(NCc1nnc(-c2ccccc2)s1)c1cnnn1C1CCOC1] | N-((5-phenyl-1,3,4- thiadiazol-2-yl)methyl)-1- (tetrahydrofuran-3-yl)-1H- 1,2,3-triazole-5- carboxamide | 357 |
| Example 307 | 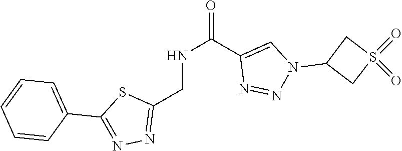 [SMILES: O=C(NCc1nnc(-c2ccccc2)s1)c1cn(C2CS(=O)(=O)C2)nn1] | 1-(1,1-dioxidothietan-3-yl)- N-((5-phenyl-1,3,4- thiadiazol-2-yl)methyl)-1H- 1,2,3-triazole-4- carboxamide | 391 |

### Example 308

### Preparation of (1-((methylsulfonyl)methyl)-N-((5-phenyl-1,3,4-thiadiazol-2-yl)methyl)-1H-1,2,3-triazole-4-carboxamide

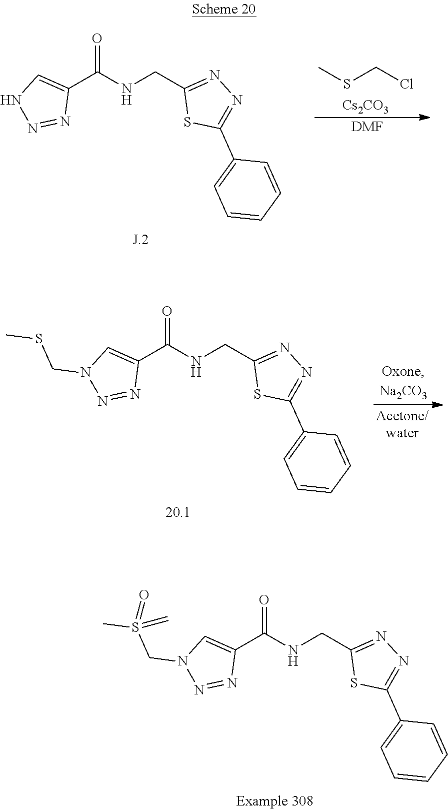

### Step 1. 1-((methylthio)methyl)-N-((5-phenyl-1,3,4-thiadiazol-2-yl)methyl)-1H-1,2,3-triazole-4-carboxamide

A mixture of N-((5-phenyl-1,3,4-thiadiazol-2-yl)methyl)-1H-1,2,3-triazole-4-carboxamide (100 mg, 0.349 mmol), Cs₂CO₃ (114 mg, 0.349 mmol) and (chloromethyl)(methyl)sulfane (0.032 mL, 0.384 mmol) in DMF (2 mL) was stirred at 60° C. for 16 hours. The mixture was filtered and the filtrate was purified via reverse phase HPLC (eluting acetonitrile in water with 0.1% TFA) to afford 1-((methylthio)methyl)-N-((5-phenyl-1,3,4-thiadiazol-2-yl)methyl)-1H-1,2,3-triazole-4-carboxamide. LCMS (ESI) m/z: 347 (M+H)⁺.

### Step 2. 1-((methylsulfonyl)methyl)-N-((5-phenyl-1,3,4-thiadiazol-2-yl)methyl)-1H-1,2,3-triazole-4-carboxamide

Na₂CO₃ (107 mg, 1.013 mmol) was added to a solution of 1-((methylthio)methyl)-N-((5-phenyl-1,3,4-thiadiazol-2-yl)methyl)-1H-1,2,3-triazole-4-carboxamide (27 mg, 0.078 mmol) in acetone (1.5 mL) at 25° C. over 2 minutes. The reaction was stirred for 1 minute at 0° C. then a solution of oxone (316 mg, 0.514 mmol) in water (0.8 mL) was added to the mixture at 25° C. and the mixture was stirred for 0.5 hours. The reaction mixture was quenched with saturated Na₂SO₃ (3 mL) and extracted with EtOAc (3×3 mL). The combined organic phases were washed with brine (10 mL), dried over anhydrous Na₂SO₄, filtered and concentrated under reduced pressure. The residue was purified via reverse phase HPLC (eluting acetonitrile in water with 0.05% NH₄OH and 10 mM NH₄HCO₃ modifier) to afford 1-((methylsulfonyl)methyl)-N-((5-phenyl-1,3,4-thiadiazol-2-yl)methyl)-1H-1,2,3-triazole-4-carboxamide. LCMS (ESI) m/z: 379 (M+H)⁺. ¹H NMR (MeOD, 400 MHz) δ 8.63 (s, 1H), 7.93-7.98 (m, 2H), 7.51-7.56 (m, 3H), 5.02 (s, 2H), 4.83-4.84 (m, 2H), 3.06 (s, 3H)

### Example 309

### Preparation of N-((5-phenyl-1,3,4-thiadiazol-2-yl)methyl)-1-(pyrimidin-4-yl)-1H-1,2,3-triazole-4-carboxamide

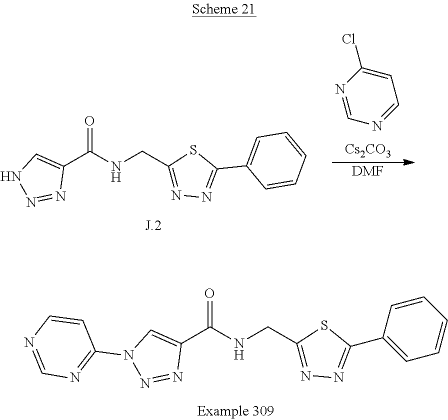

4-chloropyrimidine hydrochloride (31.6 mg, 0.210 mmol) was added to a mixture of N-((5-phenyl-1,3,4-thiadiazol-2-yl)methyl)-1H-1,2,3-triazole-4-carboxamide (60 mg, 0.210 mmol) and Cs₂CO₃ (68.3 mg, 0.210 mmol) in DMF (2 mL) at 20° C. The resulting mixture was stirred at 80° C. for 2 hours. The reaction mixture was filtered and the residue was purified via reverse phase HPLC (eluting acetonitrile in water with 0.1% TFA) to afford N-((5-phenyl-1,3,4-thiadiazol-2-yl)methyl)-2-(pyrimidin-4-yl)-2H-1,2,3-triazole-4-carboxamide. LCMS (ESI) m/z: 365 (M+H)⁺. ¹H NMR (400 MHz, MeOD) δ 9.23 (d, J=1.07 Hz, 1H), 9.05 (d, J=5.60 Hz, 1H), 8.55 (s, 1H), 8.26 (dd, J=1.19, 5.60 Hz, 1H), 7.94-8.02 (m, 2H), 7.49-7.61 (m, 3H), 5.05-5.10 (m, 2H).

Examples shown in Example Table 12 below, were prepared according to procedures analogous to those outlined in Example 309 and General Scheme 5 above using the appropriate starting materials obtained from methods described above or elsewhere, or as obtained from commercial sources.

Example Table 12

| | | | Mass |
|---|---|---|---|
| Example | Structure | Name | [M + H]⁺ |
| Example 310 | 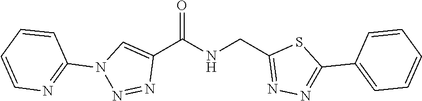 | N-((5-phenyl-1,3,4- thiadiazol-2-yl)methyl)-1- (pyridin-2-yl)-1H-1,2,3- triazole-4-carboxamide | 36 |
| Example 311 | 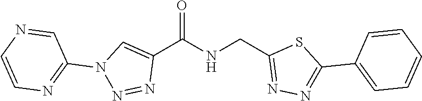 | N-((5-phenyl-1,3,4- thiadiazol-2-yl)methyl)-1- (pyrazin-2-yl)-1H-1,2,3- triazole-4-carboxamide | 365 |

### Example 312

### Preparation of 1-(1-amino-2-methyl-1-oxopropan-2-yl)-N-((5-phenyl-1,3,4-thiadiazol-2-yl)methyl)-1H-1,2,3-triazole-4-carboxamide

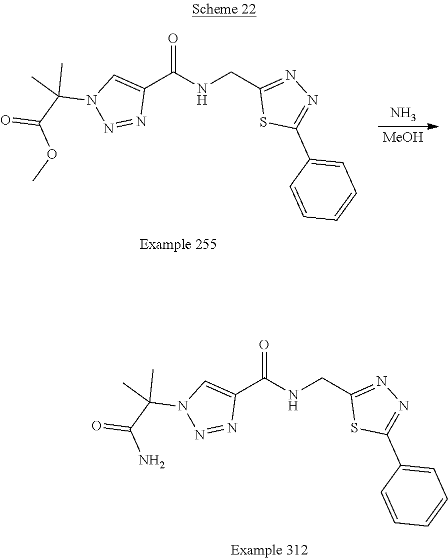

A mixture of methyl 2-methyl-2-(4-(((5-phenyl-1,3,4-thiadiazol-2-yl)methyl)carbamoyl)-1H-1,2,3-triazol-1-yl)propanoate (80 mg, 0.207 mmol) in NH₃ in MeOH (1 mL, 7 mmol, 7 M) was stirred at 25° C. for 2 h. The reaction mixture was concentrated to provide a residue which was purified via reverse phase via reverse phase HPLC (eluting acetonitrile water 0.05% NH₄OH and 10 mM NH₄HCO₃) to afford 1-(1-amino-2-methyl-1-oxopropan-2-yl)-N-((5-phenyl-1,3,4-thiadiazol-2-yl)methyl)-1H-1,2,3-triazole-4-carboxamide. LCMS (ESI) m/z: 372 (M+H)⁺. ¹H NMR (400 MHz, DMSO) δ 9.55 (t, J=6.00 Hz, 1H), 8.73 (s, 1H), 7.86-8.01 (m, 2H), 7.48-7.58 (m, 3H), 7.41 (d, J=1.00 Hz, 2H), 4.87 (d, J=5.70 Hz, 2H), 1.85 (s, 6H).

### Example 313

### Preparation of 1-(2-cyanopropan-2-yl)-N-((5-phenyl-1,3,4-thiadiazol-2-yl)methyl)-1H-1,2,3-triazole-4-carboxamide

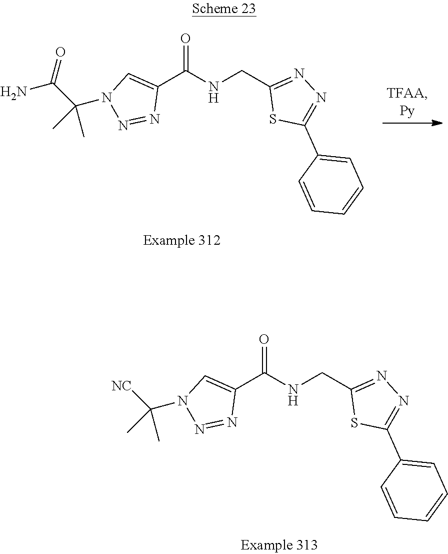 [SMILES: CC(C)(C(N)=O)n1cc(C(=O)NCc2nnc(-c3ccccc3)s2)nn1 ; "TFAA,Py" ; CC(C)(C#N)n1cc(C(=O)NCc2nnc(-c3ccccc3)s2)nn1]

Pyridine (0.02 mL, 0.242 mmol) and TFAA (0.023 mL, 0.162 mmol) were added to a solution of 1-(1-amino-2-methyl-1-oxopropan-2-yl)-N-((5-phenyl-1,3,4-thiadiazol-2-yl)methyl)-1H-1,2,3-triazole-4-carboxamide (30 mg, 0.081 mmol) in 1,4-dioxane (0.5 mL) under N₂ atmosphere. After stirring for 16 hours at 25° C. the solvent was removed under vacuum and the residue was purified via reverse phase HPLC (eluting acetonitrile in water with 0.1% TFA) to afford 1-(2-cyanopropan-2-yl)-N-((5-phenyl-1,3,4-thiadiazol-2-yl)methyl)-1H-1,2,3-triazole-4-carboxamide. LCMS (ESI) m/z: 354 (M+H)⁺. ¹H NMR (400 MHz, MeOD) δ 8.76 (s, 1H), 7.96 (dd, J=1.67, 7.75 Hz, 2H), 7.49-7.59 (m, 3H), 5.01-5.08 (m, 2H), 2.16 (s, 6H).

### Example 314

### Preparation of 1-(2,6-dimethylpyridin-3-yl)-N-((3-(pyridin-4-yl)isoxazol-5-yl)methyl)-1H-1,2,3-triazole-4-carboxamide

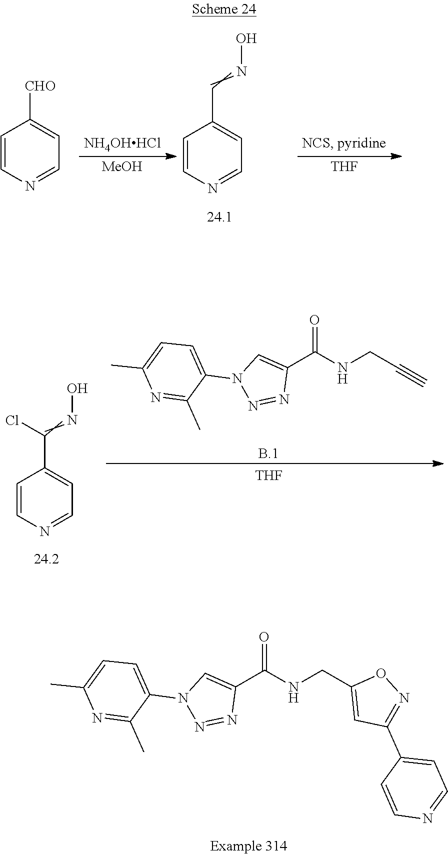

### Step 1. Preparation of isonicotinaldehyde oxime

A mixture of isonicotinaldehyde (5 g, 47 mmol) and hydroxylamine hydrochloride (3.89 g, 56.0 mmol) in MeOH (70 mL) was stirred at 80° C. for 16 hours. The reaction was cooled, and the mixture was concentrated under reduced pressure. Saturated aqueous sodium bicarbonate residue (50 mL) and the mixture was extracted with ethyl acetate (3×50 mL). The organic layer was dried with magnesium sulfate, filtered and concentrated to afford isonicotinaldehyde oxime which was used in next step without further purification. ¹H NMR (400 MHz, DMSO-d6) δ 11.82 (s, 1H), 8.54-8.62 (m, 2H), 8.17 (s, 1H), 7.51-7.56 (m, 2H)

### Step 2. Preparation of N-hydroxyisonicotinimidoyl chloride

NCS (3.61 g, 27.0 mmol) was added to a mixture of isonicotinaldehyde oxime (3 g, 24 mmol) and pyridine (0.199 mL, 2.46 mmol) in THF (40 mL) was added at 25° C. The resulting mixture was stirred at 50° C. for 5 hours. The reaction mixture was filtered and the filter cake was washed with ethyl acetate (3×20 mL) to afford N-hydroxyisonicotinimidoyl chloride which was used in next step without further purification. LCMS (ESI) m/z: 157 (M+H)⁺.

### Step 3. Preparation of 1-(2,6-dimethylpyridin-3-yl)-N-((3-(pyridin-4-yl)isoxazol-5-yl)methyl)-1H-1,2,3-triazole-4-carboxamide

A mixture of 1-(2,6-dimethylpyridin-3-yl)-N-(prop-2-yn-1-yl)-1H-1,2,3-triazole-4-carboxamide (38 mg, 0.149 mmol), TEA (0.025 mL, 0.179 mmol) and N-hydroxyisonicotinimidoyl chloride (58.3 mg, 0.372 mmol) in THF (1 mL) was stirred at 50° C. for 16 hours. The reaction was concentrated and the residue was purified via reverse phase HPLC (eluting acetonitrile in water) to afford give 1-(2,6-dimethylpyridin-3-yl)-N-((3-(pyridin-4-yl)isoxazol-5-yl)methyl)-1H-1,2,3-triazole-4-carboxamide. LCMS (ESI) m/z: 376 (M+H)⁺. ¹H NMR (400 MHz, DMSO-d6) δ 9.47 (t, J=6.14 Hz, 1H), 9.06 (s, 1H), 8.70-8.74 (m, 2H), 7.81-7.88 (m, 3H), 7.35 (d, J=8.11 Hz, 1H), 7.09 (s, 1H), 4.70 (d, J=5.96 Hz, 2H), 2.55 (s, 3H), 2.33 (s, 3H)

### Example 315

### Preparation of 1-(2-methylpyridin-3-yl)-N-((5-phenyl-1,3,4-thiadiazol-2-yl)methyl)-1H-1,2,3-triazole-4-carboxamide

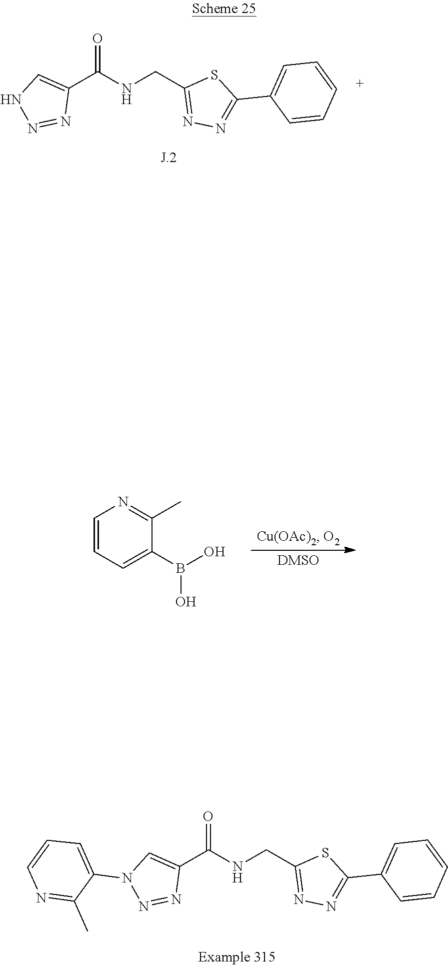

A mixture of N-((5-phenyl-1,3,4-thiadiazol-2-yl)methyl)-1H-1,2,3-triazole-4-carboxamide (45 mg, 0.157 mmol), (2-methylpyridin-3-yl)boronic acid (32.3 mg, 0.236 mmol) and copper (II) acetate (2.85 mg, 0.016 mmol) in DMSO (1 mL) was stirred at 100° C. under an O₂ atmosphere for 5 hours. The mixture was filtered and the filtrate was purified via reverse phase HPLC (eluting acetonitrile in water with 0.04% NH₄OH and 10 mM NH₄HCO₃ modifier) to afford 1-(2-methylpyridin-3-yl)-N-((5-phenyl-1,3,4-thiadiazol-2-yl)methyl)-1H-1,2,3-triazole-4-carboxamide LCMS (ESI) m/z: 378 (M+H)⁺. ¹H NMR (400 MHz, CDCl₃) δ 8.73 (d, J=3.81 Hz, 1H), 8.38 (s, 1H), 8.03 (t, J=6.38 Hz, 1H), 7.93-7.98 (m, 2H), 7.72 (dd, J=1.55, 7.99 Hz, 1H), 7.47-7.52 (m, 3H), 7.39 (dd, J=4.83, 7.81 Hz, 1H), 5.12-5.18 (m, 2H), 2.51 (s, 3H).

Examples shown in Example Table 13 below, were prepared according to procedures analogous to those outlined in Example 315 and General Scheme 5 above using the appropriate starting materials obtained from methods described above or elsewhere, or as obtained from commercial sources.

Example Table 13

| | | | Mass |
|---|---|---|---|
| Example | Structure | Name | [M + H]⁺ |
| Example 316 | 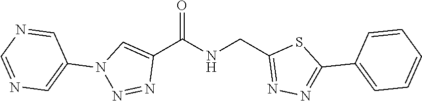 | N-((5-phenyl-1,3,4- thiadiazol-2-yl)methyl)-1- (pyrimidin-5-yl)-1H-1,2,3- triazole-4-carboxamide | 365 |

### Example 317

### Preparation of 5-(2-hydroxypropan-2-yl)-N-((5-phenyl-1,3,4-thiadiazol-2-yl)methyl)isoxazole-3-carboxamide

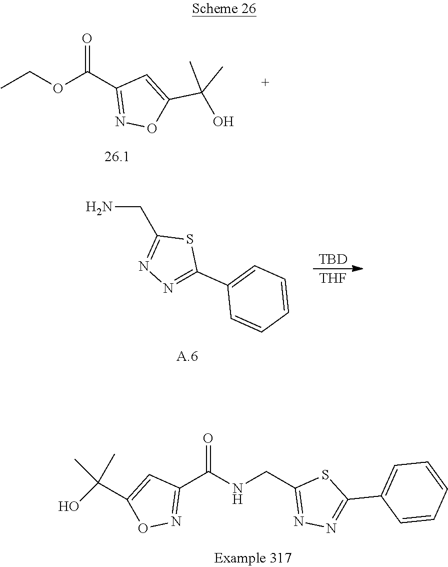

A mixture of ethyl 5-(2-hydroxypropan-2-yl)isoxazole-3-carboxylate (20 mg, 0.100 mmol), (5-phenyl-1,3,4-thiadiazol-2-yl)methanamine (33.2 mg, 0.110 mmol) and 3,4,6,7,8,9-hexahydro-2H-pyrido[1,2-a]pyrimidine (20.81 mg, 0.151 mmol) in THF (2 mL) was stirred at 70° C. for 20 hours. The mixture was filtered and the filtrate was concentrated under reduced pressure. The residue was purified via reverse phase HPLC (eluting acetonitrile in water with 0.1% TFA modifier) to afford 5-(2-hydroxypropan-2-yl)-N-((5-phenyl-1,3,4-thiadiazol-2-yl)methyl)isoxazole-3-carboxamide LCMS (ESI) m/z: 345 (M+H)⁺. ¹H NMR (400 MHz, CDCl₃) δ 7.92-7.97 (m, 2H), 7.72 (s, 1H), 7.46-7.53 (m, 3H), 6.67 (s, 1H), 5.08 (d, J=6.20 Hz, 2H), 1.67 (s, 6H)

### Example 318 and 319

### Preparation of 1-(1-amino-1-oxopropan-2-yl)-N-((5-phenyl-1,3,4-thiadiazol-2-yl)methyl)-1H-1,2,3-triazole-4-carboxamide & 1-(1-amino-1-oxopropan-2-yl)-N-((5-phenyl-1,3,4-thiadiazol-2-yl)methyl)-1H-1,2,3-triazole-4-carboxamide

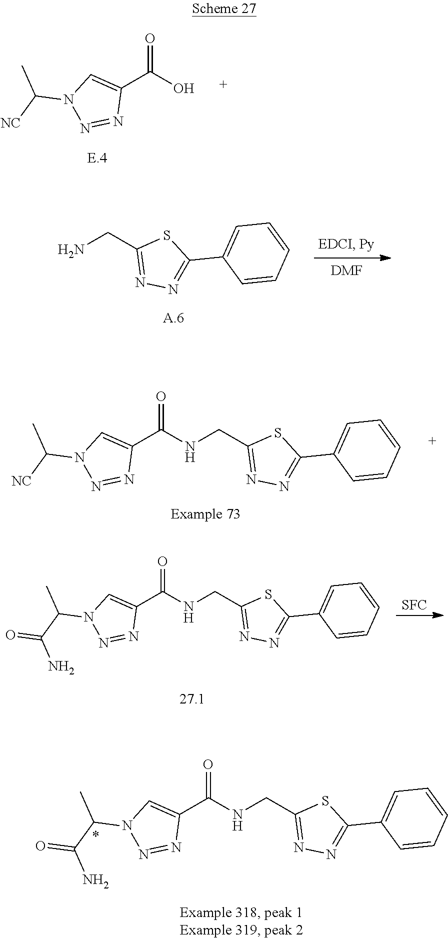

### Step 1. Preparation of racemic 1-(1-amino-1-oxopropan-2-yl)-N-((5-phenyl-1,3,4-thiadiazol-2-yl)methyl)-1H-1,2,3-triazole-4-carboxamide

HATU (172 mg, 0.451 mmol) and DIEA (0.210 mL, 1.204 mmol) were added to a solution of 1-(1-cyanoethyl)-1H-1,2,3-triazole-4-carboxylic acid (50 mg, 0.301 mmol) in DMF (1 mL) at 25° C. over 1 minute. After stirring for 2 minutes at 25° C., (5-phenyl-1,3,4-thiadiazol-2-yl)methanamine hydrochloride (109 mg, 0.361 mmol) was added to the mixture at 25° C. and the resulting mixture was stirred for another 2 hours. The mixture was filtered and the filtrate was purified via reverse phase HPLC (eluting acetonitrile in water with 0.1% TFA modifier) to afford racemic 1-(1-amino-1-oxopropan-2-yl)-N-((5-phenyl-1,3,4-thiadiazol-2-yl)methyl)-1H-1,2,3-triazole-4-carboxamide.

### Separation of Racemic 1-(1-amino-1-oxopropan-2-yl)-N-((5-phenyl-1,3,4-thiadiazol-2-yl)methyl)-1H-1,2,3-triazole-4-carboxamide

The racemic mixture of 1-(1-amino-1-oxopropan-2-yl)-N-((5-phenyl-1,3,4-thiadiazol-2-yl)methyl)-1H-1,2,3-triazole-4-carboxamide was resolved by chiral SFC purification (DAICEL CHIRALCEL OD 250×30 mm column, 100% IPA w/0.1% NH₄OH as cosolvent).

### Example 318 Peak 1

LCMS (ESI) m/z: 358 (M+H)⁺. ¹H NMR (400 MHz, MeOD) δ 8.56 (s, 1H), 7.92-7.97 (m, 2H), 7.49-7.56 (m, 3H), 5.51 (q, J=7.31 Hz, 1H), 5.01 (s, 2H), 1.84 (d, J=7.27 Hz, 3H)

### Example 319 Peak 2

LCMS (ESI) m/z: 358 (M+H)⁺. ¹H NMR (400 MHz, MeOD) δ 8.56 (s, 1H), 7.92-7.97 (m, 2H), 7.49-7.56 (m, 3H), 5.51 (q, J=7.31 Hz, 1H), 5.01 (s, 2H), 1.84 (d, J=7.27 Hz, 3H)

### Example 320

### Preparation of 1-(1-amino-2-methyl-1-oxopropan-2-yl)-N-((5-(pyridin-4-yl)-1,3,4-thiadiazol-2-yl)methyl)-1H-1,2,3-triazole-4-carboxamide

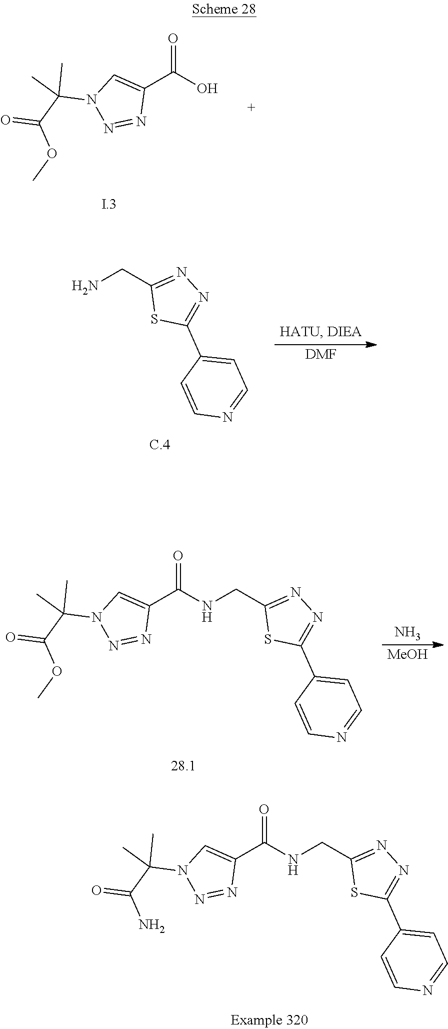

### Step 1. Preparation of methyl 2-methyl-2-(4-(((5-(pyridin-4-yl)-1,3,4-thiadiazol-2-yl)methyl)carbamoyl)-1H-1,2,3-triazol-1-yl)propanoate

A mixture of 1-(1-methoxy-2-methyl-1-oxopropan-2-yl)-1H-1,2,3-triazole-4-carboxylic acid (158 mg, 0.741 mmol), (5-(pyridin-4-yl)-1,3,4-thiadiazol-2-yl)methanamine (142 mg, 0.741 mmol), DIEA (0.259 mL, 1.482 mmol) and HATU (282 mg, 0.741 mmol) in DCM (5 mL) was stirred at 25° C. for 16 hours. The reaction mixture was quenched with sat. NH₄Cl (20 mL) and extracted with EtOAc (3×10 mL). The combined organic phases were washed with brine (20 mL), dried over anhydrous Na₂SO₄, filtered and concentrated under reduced pressure. The residue was purified by flash silica gel chromatography (eluting ethyl acetate in petroleum ether) to afford methyl 2-methyl-2-(4-(((5-(pyridin-4-yl)-1,3,4-thiadiazol-2-yl)methyl)carbamoyl)-1H-1,2,3-triazol-1-yl)propanoate. LCMS (ESI) m/z: 388 (M+H)⁺.

### Step 2: Preparation of 1-(1-amino-2-methyl-1-oxopropan-2-yl)-N-((5-(pyridin-4-yl)-1,3,4-thiadiazol-2-yl)methyl)-1H-1,2,3-triazole-4-carboxamide

A mixture of methyl 2-methyl-2-(4-(((5-(pyridin-4-yl)-1,3,4-thiadiazol-2-yl)methyl)carbamoyl)-1H-1,2,3-triazol-1-yl)propanoate (25 mg, 0.065 mmol) and NH₃ in MeOH (1.5 mL, 0.065 mmol) in MeOH (1 mL) was stirred at 25° C. for 12 hours. The reaction mixture was concentrated to afford 1-(1-amino-2-methyl-1-oxopropan-2-yl)-N-((5-(pyridin-4-yl)-1,3,4-thiadiazol-2-yl)methyl)-1H-1,2,3-triazole-4-carboxamide. LCMS (ESI) m/z: 373 (M+H)⁺. 1H NMR (400 MHz, MeOD) δ 8.72 (d, J=5.25 Hz, 2H), 8.57 (s, 1H), 7.97 (d, J=5.36 Hz, 2H), 5.05 (s, 2H), 1.95 (s, 6H).

Examples shown in Example Table 14 below, were prepared according to procedures analogous to those outlined in Example 320 and General Scheme 2 above using the appropriate starting materials obtained from methods described above or elsewhere, or as obtained from commercial sources.

Example Table 14

| | | | Mass |
|---|---|---|---|
| Example | Structure | Name | [M + H]⁺ |
| Example 321 | 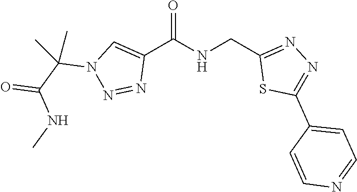 | 1-(2-methyl-1- (methylamino)-1- oxopropan-2-yl)-N-((5- (pyridin-4-yl)-1,3,4- thiadiazol-2-yl)methyl)-1H- 1,2,3-triazole-4- carboxamide | 387 |
| Example 322 | 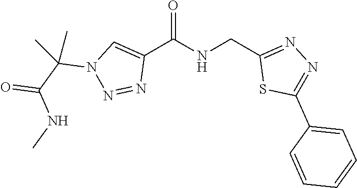 | 1-(2-methyl-1- (methylamino)-1- oxopropan-2-yl)-N-((5- phenyl-1,3,4-thiadiazol-2- yl)methyl)-1H-1,2,3- triazole-4-carboxamide | 386 |

### Example 323

### Preparation of 1-(2-hydroxy-2-methylpropyl)-N-((5-phenyl-1,3,4-thiadiazol-2-yl)methyl)-1H-1,2,3-triazole-4-carboxamide

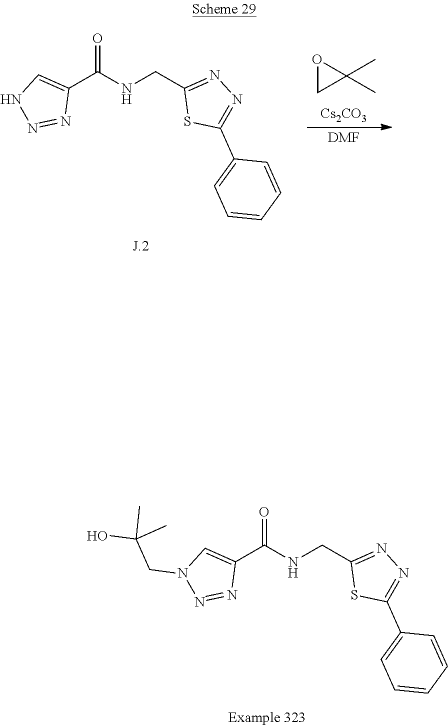

A mixture of N-((5-phenyl-1,3,4-thiadiazol-2-yl)methyl)-1H-1,2,3-triazole-4-carboxamide (100 mg, 0.349 mmol), 2,2-dimethyloxirane (0.047 mL, 0.524 mmol) and Cs₂CO₃ (171 mg, 0.524 mmol) in DMF (2 mL) was stirred at 100° C. for 16 hours. The mixture was filtered and the filtrate was purified via reverse phase HPLC (eluting acetonitrile in water with 0.05% NH₄OH+10 mM NH₄HCO₃ modifier) to afford 1-(2-hydroxy-2-methylpropyl)-N-((5-phenyl-1,3,4-thiadiazol-2-yl)methyl)-1H-1,2,3-triazole-4-carboxamide. LCMS (ESI) m/z: 359 (M+H)⁺. ¹H NMR (400 MHz, MeOD) δ 8.41 (s, 1H), 7.95 (dd, J=1.61, 7.69 Hz, 2H), 7.49-7.56 (m, 3H), 5.01 (s, 2H), 4.42 (s, 2H), 1.20 (s, 6H)

### Example 324

### Preparation of N-((5-cyclohexyl-1,3,4-thiadiazol-2-yl)methyl)-1-(2,6-dimethylpyridin-3-yl)-1H-1,2,3-triazole-4-carboxamide

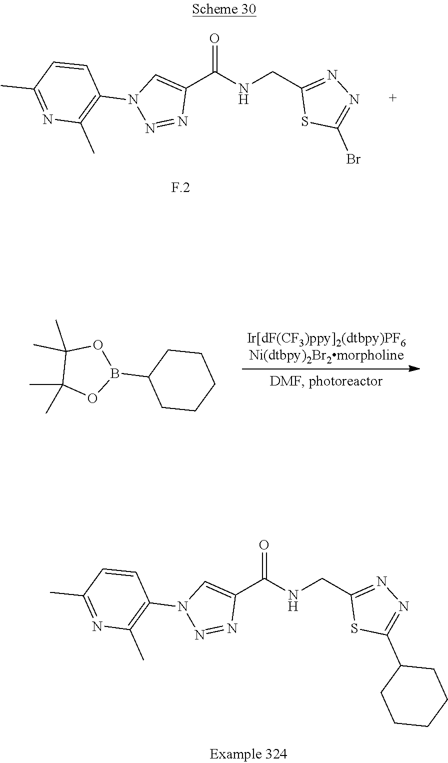 [SMILES: Cc1ccc(-n2cc(C(=O)NCc3nnc(Br)s3)nn2)c(C)n1 ; CC1(C)OB(C2CCCCC2)OC1(C)C ; Cc1ccc(-n2cc(C(=O)NCc3nnc(C4CCCCC4)s3)nn2)c(C)n1]

A mixture of N-((5-bromo-1,3,4-thiadiazol-2-yl)methyl)-1-(2,6-dimethylpyridin-3-yl)-1H-1,2,3-triazole-4-carboxamide (50 mg, 0.13 mmol), 2-cyclohexyl-4,4,5,5-tetramethyl-1,3,2-dioxaborolane (0.057 mL, 0.254 mmol), morpholine (0.017 mL, 0.190 mmol), (Ir[dF(CF₃)ppy]₂(dtbpy))PF₆ (1.423 mg, 1.268 μmol), and Ni(dtbpy)Br₂ (3.09 mg, 6.34 μmol) in DMF (1 mL) was degassed and backfilled with N₂ (three times). The mixture was stirred at 25° C. for 4 hours under irradiation of 450 nm lights in a photoreactor. The mixture was filtered and the filtrate was purified via reverse phase HPLC (eluting acetonitrile in water with (0.05% NH₄OH+10 mM NH₄HCO₃) to afford N-((5-cyclohexyl-1,3,4-thiadiazol-2-yl)methyl)-1-(2,6-dimethylpyridin-3-yl)-1H-1,2,3-triazole-4-carboxamide. LCMS (ESI) m/z: 398 (M+H)⁺. ¹H NMR (400 MHz, CDCl₃) δ 8.32 (s, 1H), 7.87-7.99 (m, 1H), 7.60 (d, J=8.11 Hz, 1H), 7.23 (d, J=8.23 Hz, 1H), 5.07 (d, J=6.32 Hz, 2H), 3.08-3.22 (m, 1H), 2.68 (s, 3H), 2.47 (s, 3H), 2.12-2.20 (m, 2H), 1.83-1.89 (m, 2H), 1.69-1.81 (m, 2H), 1.39-1.47 (m, 2H), 1.20-1.37 (m, 2H).

Examples shown in Example Table 15 below, were prepared according to procedures analogous to those outlined in Example 324 and General Scheme 4 above using the appropriate starting materials obtained from methods described above or elsewhere, or as obtained from commercial sources.

Example Table 15

| | | | Mass |
|---|---|---|---|
| Example | Structure | Name | [M + H]⁺ |
| Example 325 | 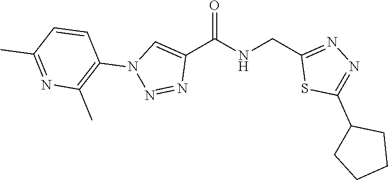 | N-((5-cyclopentyl-1,3,4- thiadiazol-2-yl)methyl)-1- (2,6-dimethylpyridin-3-yl)- 1H-1,2,3-triazole-4- carboxamide | 384 |
| Example 326 | 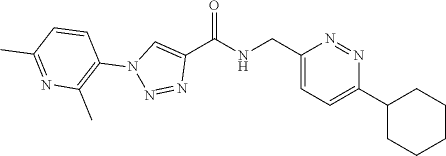 | N-((6-cyclohexylpyridazin- 3-yl)methyl)-1-(2,6- dimethylpyridin-3-yl)-1H- 1,2,3-triazole-4- carboxamide | 392 |

### Example 327

### Preparation of 1-(2-methyl-6-(oxetan-3-yl)pyridin-3-yl)-N-((5-phenyl-1,3,4-thiadiazol-2-yl)methyl)-1H-1,2,3-triazole-4-carboxamide

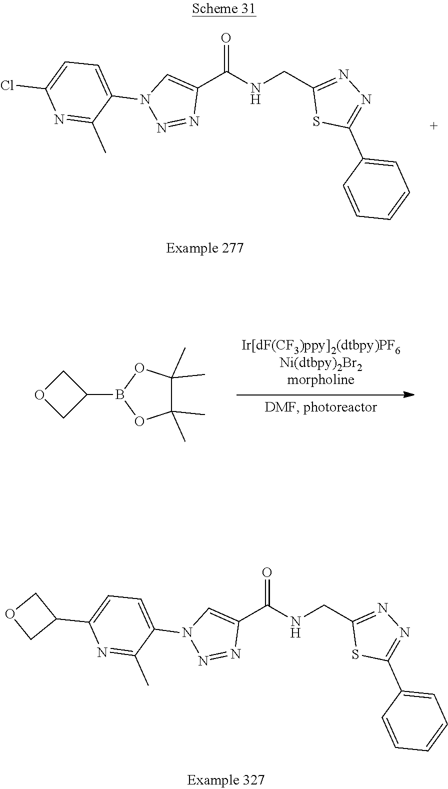 [SMILES: Cc1nc(Cl)ccc1-n1cc(C(=O)NCc2nnc(-c3ccccc3)s2)nn1 ; CC1(C)OB(C2COC2)OC1(C)C ; Cc1nc(C2COC2)ccc1-n1cc(C(=O)NCc2nnc(-c3ccccc3)s2)nn1]

A mixture of 4,4,5,5-tetramethyl-2-(oxetan-3-yl)-1,3,2-dioxaborolane (57.2 mg, 0.311 mmol), 1-(6-chloro-2-methylpyridin-3-yl)-N-((5-phenyl-1,3,4-thiadiazol-2-yl)methyl)-1H-1,2,3-triazole-4-carboxamide (64 mg, 0.155 mmol), morpholine (0.020 mL, 0.233 mmol), Ir[dF(CF₃)ppy]₂(dtbpy))PF₆ (1.743 mg, 1.554 μmol), and Ni(dtbpy)Br₂ (3.78 mg, 7.77 μmol) in DMF (1 mL) was degassed and backfilled with N₂ (three times). The mixture was stirred at 25° C. for 4 hours under irradiation of 450 nm lights in a photoreactor. The mixture was filtered and the filtrate was purified via reverse phase HPLC (eluting acetonitrile in water with 0.05% NH₄OH+10 mM NH₄HCO₃ modifier) to afford 1-(2-methyl-6-(oxetan-3-yl)pyridin-3-yl)-N-((5-phenyl-1,3,4-thiadiazol-2-yl)methyl)-1H-1,2,3-triazole-4-carboxamide. LCMS (ESI) m/z: 434 (M+H)⁺. ¹H NMR (400 MHz, MeOD) δ 8.79 (s, 1H), 7.94-7.98 (m, 2H), 7.86 (d, J=8.11 Hz, 1H), 7.51-7.57 (m, 3H), 7.44 (d, J=8.11 Hz, 1H), 5.07-5.11 (m, 2H), 5.05 (s, 2H), 4.98 (t, J=6.26 Hz, 2H), 4.49-4.57 (m, 1H), 2.48 (s, 3H).

### Example 328

### Preparation of 3-(2-hydroxypropan-2-yl)-N-((5-phenyl-1,3,4-thiadiazol-2-yl)methyl)isoxazole-5-carboxamide

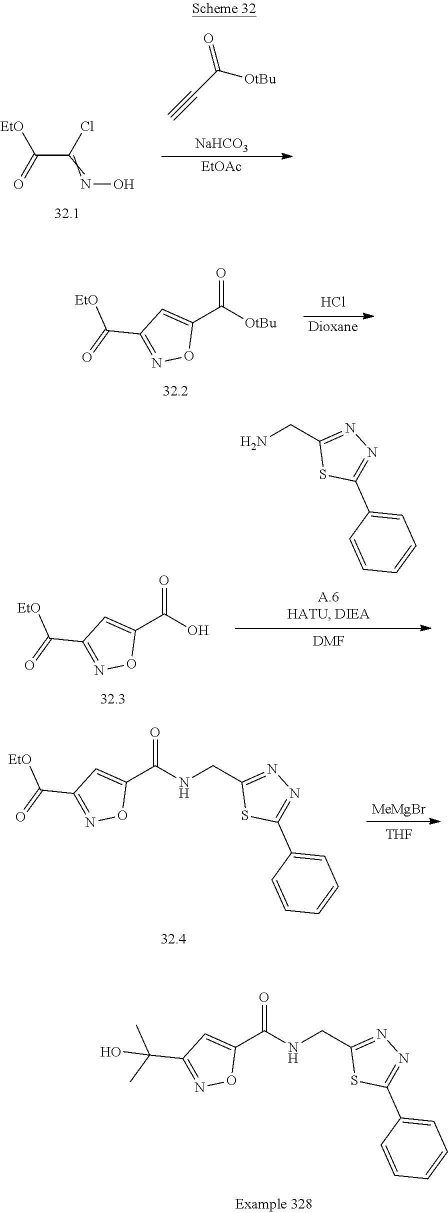

### Step 1. Preparation of 5-(tert-butyl) 3-ethyl isoxazole-3,5-dicarboxylate

A mixture of ethyl (E)-2-chloro-2-(hydroxyimino)acetate (4.81 g, 31.7 mmol) and tert-butyl propiolate (2.176 mL, 15.85 mmol), NaHCO₃ (2.66 g, 31.7 mmol) in EtOAc (8 mL) was stirred at 100° C. for 1 hour in a microwave reactor. The mixture was filtered and concentrated. The residue was purified by silica gel chromatography (eluting ethyl acetate in petroleum ether) to afford 5-(tert-butyl) 3-ethyl isoxazole-3,5-dicarboxylate. LCMS (ESI) m/z: 242 (M+H)⁺.

### Step 2. Preparation of 3-(ethoxycarbonyl)isoxazole-5-carboxylic acid

A mixture of 5-(tert-butyl) 3-ethyl isoxazole-3,5-dicarboxylate (3.6 g, 14.92 mmol) and HCl in (11.19 mL, 44.8 mmol, 4M in dioxane) in 1,4-dioxane (20 mL) was stirred at 25° C. for 16 hours. The mixture was concentrated under reduced pressure and the residue was purified by silica gel chromatography (eluting acetonitrile in water) to afford 3-(ethoxycarbonyl)isoxazole-5-carboxylic acid. LCMS (ESI) m/z: 186 (M+H)⁺.

### Step 3. Preparation of ethyl 5-(((5-phenyl-1,3,4-thiadiazol-2-yl)methyl)carbamoyl)isoxazole-3-carboxylate

A mixture of 3-(ethoxycarbonyl)isoxazole-5-carboxylic acid (500 mg, 2.70 mmol), HATU (1540 mg, 4.05 mmol), DIEA (1.415 mL, 8.10 mmol) and (5-phenyl-1,3,4-thiadiazol-2-yl)methanamine hydrochloride (812 mg, 2.70 mmol) in DCM (8 mL) was stirred at 25° C. for 16 hours. The mixture was filtered and the filtrate was concentrated under reduced pressure. The residue was purified by silica gel chromatography (eluting ethyl acetate in petroleum ether) to afford ethyl 5-(((5-phenyl-1,3,4-thiadiazol-2-yl)methyl)carbamoyl)isoxazole-3-carboxylate. LCMS (ESI) m/z: 359 (M+H)⁺.

### Step 4. Preparation of 3-(2-hydroxypropan-2-yl)-N-((5-phenyl-1,3,4-thiadiazol-2-yl)methyl)isoxazole-5-carboxamide

Methylmagnesium bromide (0.558 mL, 1.674 mmol) was added to a mixture of ethyl 5-(((5-phenyl-1,3,4-thiadiazol-2-yl)methyl)carbamoyl)isoxazole-3-carboxylate (200 mg, 0.558 mmol) in THF (8 mL) was added at 0° C. The resulting mixture was stirred at 25° C. for 2 hours. The reaction mixture was quenched with sat.NH₄Cl (5 mL) and extracted with EtOAc (3×5 mL). The combined organic phases were washed with brine (2 mL), dried over anhydrous Na₂SO₄, filtered and concentrated under reduced pressure. The residue was purified via reverse phase HPLC (eluting acetonitrile in water with 0.05% NH₄OH+10 mM NH₄HCO₃ modifier) to afford 3-(2-hydroxypropan-2-yl)-N-((5-phenyl-1,3,4-thiadiazol-2-yl)methyl)isoxazole-5-carboxamide. LCMS (ESI) m/z: 345 (M+H)⁺. ¹H NMR (400 MHz, MeOD) δ 7.94-7.98 (m, 2H), 7.50-7.55 (m, 3H), 7.07 (s, 1H), 4.98 (s, 2H), 1.58 (s, 6H).

### Example 329 Peak 1 & 330 Peak 2

### Preparation of 3-(1-hydroxyethyl)-N-((5-phenyl-1,3,4-thiadiazol-2-yl)methyl)isoxazole-5-carboxamide

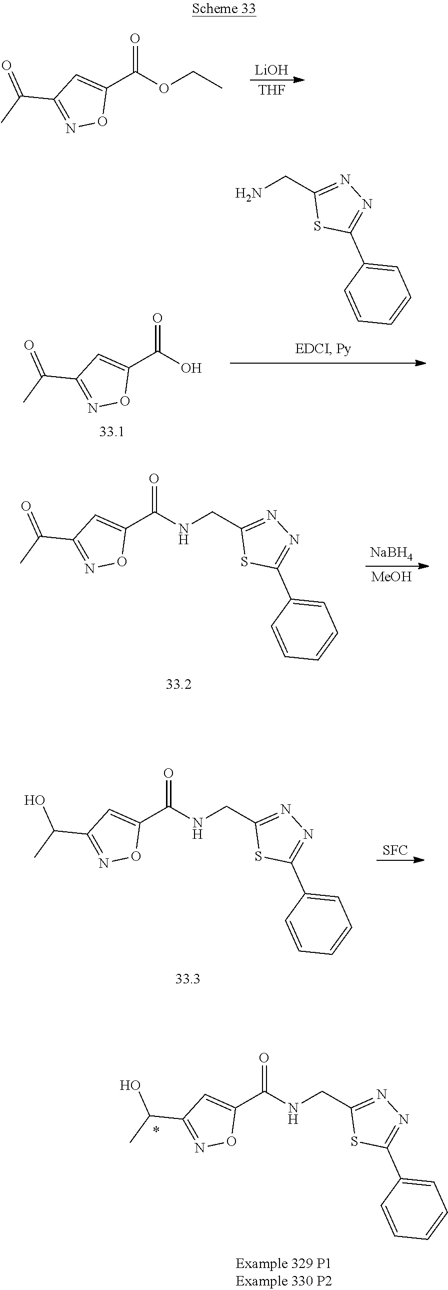

### Step 1. Preparation of 3-acetylisoxazole-5-carboxylic acid

A mixture of ethyl 3-acetylisoxazole-5-carboxylate (200 mg, 1.092 mmol) and LiOH·H₂O (55.0 mg, 1.310 mmol) in THF (2 mL) and water (0.5 mL) was stirred at 25° C. for 2 hours. The pH was adjusted by 4M HCl (0.1 mL, pH 4) and then the mixture was diluted with H₂O (2 mL) and extracted with EtOAc (3×2 mL). The combined organic phases were washed with brine (2 mL), dried over anhydrous Na₂SO₄, filtered and concentrated under reduced pressure to give the crude 3-acetylisoxazole-5-carboxylic acid, which was used directly without further purification.

### Step 2. Preparation of 3-acetyl-N-((5-phenyl-1,3,4-thiadiazol-2-yl)methyl)isoxazole-5-carboxamide

Pyridine (0.501 mL, 6.19 mmol) and 3-(((ethylimino)methylene)amino)-N,N-dimethylpropan-1-amine hydrochloride (222 mg, 1.16 mmol) were added to a solution of 3-acetylisoxazole-5-carboxylic acid (120 mg, 0.774 mmol) in DMF (3 mL) at 25° C. over 1 minute. After stirring for 2 minutes at 25° C., (5-phenyl-1,3,4-thiadiazol-2-yl)methanamine trihydrochloride (233 mg, 0.774 mmol) was added to the mixture at 25° C. The resulting mixture was stirred for another 2 hours. The mixture was filtered and the filtrate was purified via reverse phase HPLC (eluting acetonitrile in water with 0.04% NH₄OH+10 mM NH₄HCO₃ modifier) to afford 3-acetyl-N-((5-phenyl-1,3,4-thiadiazol-2-yl)methyl)isoxazole-5-carboxamide. LCMS (ESI) m/z: 329 (M+H)⁺.

### Step 3. Preparation of 3-(1-hydroxyethyl)-N-((5-phenyl-1,3,4-thiadiazol-2-yl)methyl)isoxazole-5-carboxamide

NaBH₄ (2.42 mg, 0.0640 mmol) was added to a solution of 3-acetyl-N-((5-phenyl-1,3,4-thiadiazol-2-yl)methyl)isoxazole-5-carboxamide (70 mg, 0.213 mmol) in MeOH (1 mL) at 0° C. The mixture was stirred at 25° C. for another 2 hours. The mixture was concentrated under reduced pressure. The residue was purified via reverse phase HPLC (acetonitrile in water with 0.1% TFA modifier) to afford 3-(1-hydroxyethyl)-N-((5-phenyl-1,3,4-thiadiazol-2-yl)methyl)isoxazole-5-carboxamide. LCMS (ESI) m/z: 331 (M+H)⁺.

### Step 4. Separation of racemic 3-(1-hydroxyethyl)-N-((5-phenyl-1,3,4-thiadiazol-2-yl)methyl)isoxazole-5-carboxamide

The racemic mixture of 3-(1-hydroxyethyl)-N-((5-phenyl-1,3,4-thiadiazol-2-yl)methyl)isoxazole-5-carboxamide was resolved by chiral SFC purification (Daicel Chiralpak AD 250×30 mm column, 40% EtOH w/0.05% DEA as cosolvent).

### Example 329, Peak 1

LCMS (ESI) m/z: 331 (M+H)⁺. ¹H NMR (400 MHz, MeOD) δ7.93-7.98 (m, 2H), 7.50-7.57 (m, 3H), 7.06 (s, 1H), 4.99 (s, 2H), 4.94-4.98 (m, 1H), 1.52 (d, J=6.56 Hz, 3H)

### Example 330, Peak 2

LCMS (ESI) m/z: 331 ((M+H)⁺. ¹H NMR (400 MHz, MeOD) δ7.93-7.98 (m, 2H), 7.50-7.57 (m, 3H), 7.06 (s, 1H), 4.99 (s, 2H), 4.94-4.98 (m, 1H), 1.52 (d, J=6.56 Hz, 3H)

### Example 331

### 2,2-difluoro-3-(4-(((5-phenyl-1,3,4-thiadiazol-2-yl)methyl)carbamoyl)-1H-1,2,3-triazol-1-yl)propanoic acid

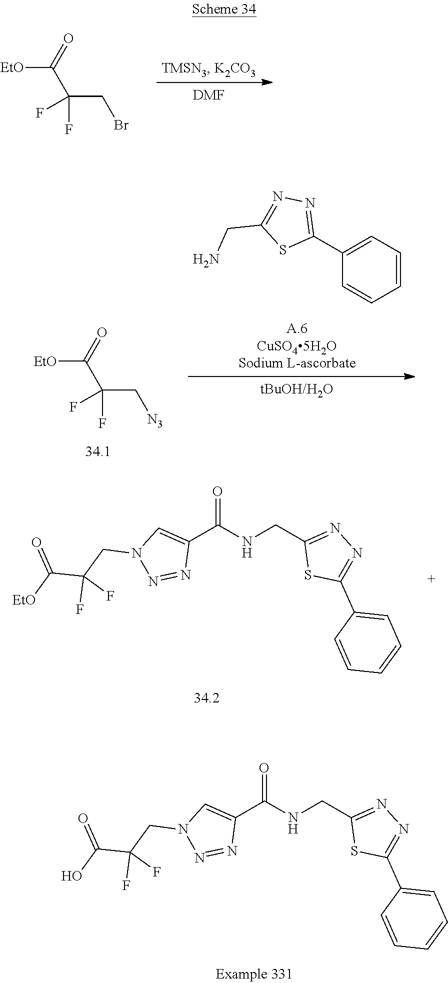

### Step 1. Preparation of ethyl 3-azido-2,2-difluoropropanoate

A mixture of ethyl 3-bromo-2,2-difluoropropanoate (0.064 mL, 0.461 mmol), K₂CO₃ (191 mg, 1.382 mmol) and TMS-N₃ (0.092 mL, 0.691 mmol) in DMF (1.5 mL) was stirred at 80° C. for 2 hours. The mixture was filtered and the filtrate was concentrated under reduced pressure to afford ethyl 3-azido-2,2-difluoropropanoate which was used without further purification.

### Step 2. Preparation of 2,2-difluoro-3-(4-(((5-phenyl-1,3,4-thiadiazol-2-yl)methyl)carbamoyl)-1H-1,2,3-triazol-1-yl)propanoic acid

CuSO₄ (7.13 mg, 0.045 mmol) and a solution of (+)-Sodium L-ascorbate (17.70 mg, 0.089 mmol) in water (0.4 mL) were added to a solution of ethyl 3-azido-2,2-difluoropropanoate (80 mg, 0.447 mmol) and N-((5-phenyl-1,3,4-thiadiazol-2-yl)methyl)propiolamide (163 mg, 0.670 mmol) in DMF (2 mL) at 25° C. After stirring for 2 hours at 50° C. the mixture was cooled to room temperature, filtered and the filtrate was concentrated under reduced pressure. The residue was purified via reverse phase HPLC (eluting acetonitrile in water with 0.1% TFA) to afford give ethyl 2,2-difluoro-3-(4-(((5-phenyl-1,3,4-thiadiazol-2-yl)methyl)carbamoyl)-1H-1,2,3-triazol-1-yl)propanoate and 2,2-difluoro-3-(4-(((5-phenyl-1,3,4-thiadiazol-2-yl)methyl)carbamoyl)-1H-1,2,3-triazol-1-yl)propanoic acid.

### Example 331

LCMS (ESI) m/z: 395 (M+H)⁺. ¹H NMR (400 MHz, DMSO-d₆) δ 9.57-9.68 (m, 1H), 8.63 (s, 1H), 7.83-8.04 (m, 2H), 7.46-7.60 (m, 3H), 5.35 (s, 2H), 4.87 (d, J=5.25 Hz, 2H)

### Example 332

### Preparation of 1-(3-amino-2,2-difluoro-3-oxopropyl)-N-((5-phenyl-1,3,4-thiadiazol-2-yl)methyl)-1H-1,2,3-triazole-4-carboxamide

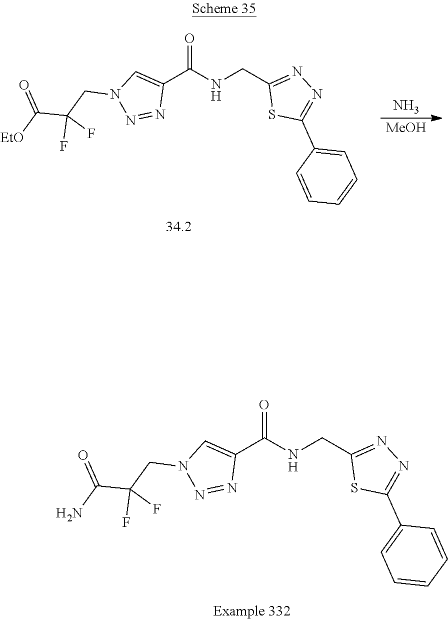

A mixture of ethyl 2,2-difluoro-3-(4-(((5-phenyl-1,3,4-thiadiazol-2-yl)methyl)carbamoyl)-1H-1,2,3-triazol-1-yl)propanoate (70 mg, 0.166 mmol) and NH₃ (0.237 mL, 1.657 mmol, 7 M in MeOH) in MeOH (2 mL) was stirred at 25° C. for 2 hours. The mixture was purified via reverse phase HPLC (eluting acetonitrile in water with 0.05% NH₄OH+10 mM NH₄HCO₃ modifier) to afford 1-(3-amino-2,2-difluoro-3-oxopropyl)-N-((5-phenyl-1,3,4-thiadiazol-2-yl)methyl)-1H-1,2,3-triazole-4-carboxamide. LCMS (ESI) m/z: 394 (M+H)⁺. ¹H NMR (400 MHz, DMSO-d6) δ 9.64 (t, J=6.02 Hz, 1H), 8.64 (s, 1H), 8.38 (s, 1H), 8.18 (s, 1H), 7.96 (dd, J=1.79, 7.63 Hz, 2H), 7.48-7.61 (m, 3H), 5.27 (t, J=14.78 Hz, 2H), 4.87 (d, J=5.96 Hz, 2H)

### Example 333

### Preparation of 6-methyl-5-(4-(((5-phenyl-1,3,4-thiadiazol-2-yl)methyl)carbamoyl)-1H-1,2,3-triazol-1-yl)picolinamide

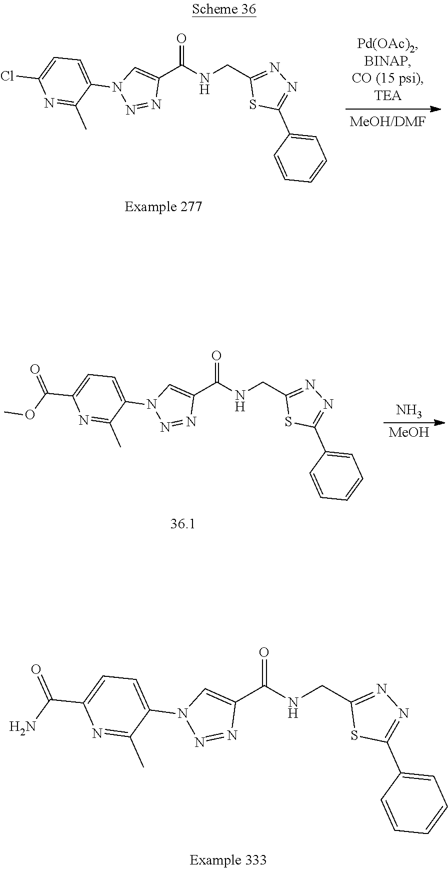

### Step 1. Preparation of methyl 6-methyl-5-(4-(((5-phenyl-1,3,4-thiadiazol-2-yl)methyl)carbamoyl)-1H-1,2,3-triazol-1-yl)picolinate

A mixture of 1-(6-chloro-2-methylpyridin-3-yl)-N-((5-phenyl-1,3,4-thiadiazol-2-yl)methyl)-1H-1,2,3-triazole-4-carboxamide (100 mg, 0.243 mmol), TEA (0.102 mL, 0.728 mmol), (R)-(+)-2,2′-bis(diphenylphosphino)-1,1′-binaphthyl (7.56 mg, 0.012 mmol) and palladium(II) acetate (2.73 mg, 0.012 mmol) in MeOH (3 mL) and DMF (3 mL) was stirring for 16 hours at 80° C. under an atmosphere of carbon monoxide (15 psi). The mixture was filtered and the filtrate was concentrated under reduced pressure. The residue was purified by preparative TLC on silica gel (eluting ethyl acetate) to afford methyl 6-methyl-5-(4-(((5-phenyl-1,3,4-thiadiazol-2-yl)methyl)carbamoyl)-1H-1,2,3-triazol-1-yl)picolinate as a solid. LCMS (ESI) m/z: 436 (M+H)⁺.

### Step 2. Preparation of 6-methyl-5-(4-(((5-phenyl-1,3,4-thiadiazol-2-yl)methyl)carbamoyl)-1H-1,2,3-triazol-1-yl)picolinamide

A mixture of methyl 6-methyl-5-(4-(((5-phenyl-1,3,4-thiadiazol-2-yl)methyl)carbamoyl)-1H-1,2,3-triazol-1-yl)picolinate (20 mg, 0.046 mmol) in NH₃ (1 mL, 7M in MeOH) in MeOH (1 mL) was stirred for 16 hours at 25° C. The mixture was filtered and the filtrate was concentrated under reduced pressure. The residue was purified by reverse phase HPLC (eluting acetonitrile in water with 0.1% TFA as a modifier) to afford 6-methyl-5-(4-(((5-phenyl-1,3,4-thiadiazol-2-yl)methyl)carbamoyl)-1H-1,2,3-triazol-1-yl)picolinamide. LCMS (ESI) m/z: 421 (M+H)⁺. ¹H NMR (DMSO, 400 MHz) δ 9.73-9.80 (m, 1H), 9.20 (s, 1H), 8.20-8.24 (m, 1H), 8.18 (d, J=8.11 Hz, 1H), 8.06-8.11 (m, 1H), 7.94-8.02 (m, 2H), 7.81-7.86 (m, 1H), 7.51-7.59 (m, 3H), 4.93 (d, J=6.08 Hz, 2H), 3.36 (s, 3H).

### Example 334

### 1-(6-(2-hydroxypropan-2-yl)-2-methylpyridin-3-yl)-N-((5-phenyl-1,3,4-thiadiazol-2-yl)methyl)-1H-1,2,3-triazole-4-carboxamide

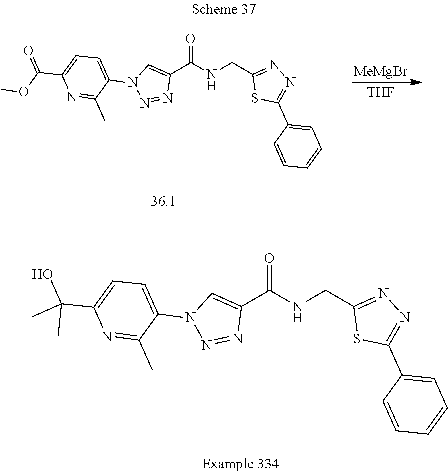 [SMILES: "MeMgBr" ; COC(=O)c1ccc(-n2cc(C(=O)NCc3nnc(-c4ccccc4)s3)nn2)c(C)n1 ; "THF" ; Cc1nc(C(C)(C)O)ccc1-n1cc(C(=O)NCc2nnc(-c3ccccc3)s2)nn1]

Methylmagnesium bromide (0.115 mL, 0.344 mmol, 3 M in THF) was added to a solution of methyl 6-methyl-5-(4-(((5-phenyl-1,3,4-thiadiazol-2-yl)methyl)carbamoyl)-1H-1,2,3-triazol-1-yl)picolinate (30 mg, 0.069 mmol)) in THF (1 mL) at 0° C. over 1 minute. The resulting mixture was stirred at for 1 hour. The mixture was filtered and the filtrate was concentrated under reduced pressure. The residue was purified via reverse phase HPLC (acetonitrile in water with 0.1% TFA modifier) to afford 1-(6-(2-hydroxypropan-2-yl)-2-methylpyridin-3-yl)-N-((5-phenyl-1,3,4-thiadiazol-2-yl)methyl)-1H-1,2,3-triazole-4-carboxamide. LCMS (ESI) m/z: 436 (M+H)⁺. ¹H NMR (CDCl₃, 400 MHz) δ 8.41 (s, 1H), 8.09-8.18 (m, 1H), 7.91-7.96 (m, 2H), 7.75 (d, J=8.23 Hz, 1H), 7.44-7.54 (m, 4H), 5.16 (d, J=6.32 Hz, 2H), 2.52 (s, 3H), 1.61 (s, 6H)

### Example 335

### Preparation of 1-(6-cyano-2-methylpyridin-3-yl)-N-((5-phenyl-1,3,4-thiadiazol-2-yl)methyl)-1H-1,2,3-triazole-4-carboxamide

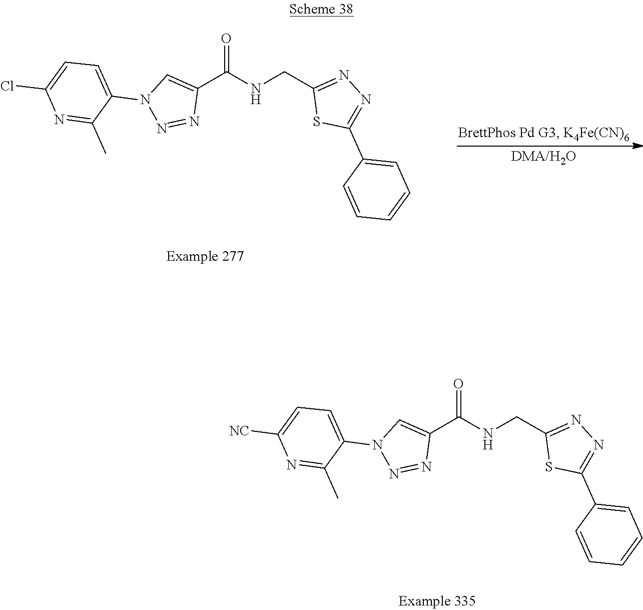 [SMILES: Cc1nc(Cl)ccc1-n1cc(C(=O)NCc2nnc(-c3ccccc3)s2)nn1 ; Cc1nc(C#N)ccc1-n1cc(C(=O)NCc2nnc(-c3ccccc3)s2)nn1]

A mixture of 1-(6-chloro-2-methylpyridin-3-yl)-N-((5-phenyl-1,3,4-thiadiazol-2-yl)methyl)-1H-1,2,3-triazole-4-carboxamide (50 mg, 0.121 mmol), potassium hexacyanoferrate(II) trihydrate (25.6 mg, 0.061 mmol) and BrettPhos Pd G3 (11.00 mg, 0.012 mmol) in DMA (3 mL) and water (0.5 mL) was stirred at 100° C. for 4 hours. The reaction mixture filtered and purified via reverse phase HPLC (eluting acetonitrile in water with 0.1% TFA modifier) to afford 1-(6-cyano-2-methylpyridin-3-yl)-N-((5-phenyl-1,3,4-thiadiazol-2-yl)methyl)-1H-1,2,3-triazole-4-carboxamide. LCMS (ESI) m/z: 403 (M+H)⁺. ¹H NMR (CDCl₃, 400 MHz) δ 8.44 (s, 1H), 8.00-8.07 (m, 1H), 7.92-7.99 (m, 2H), 7.91 (d, J=8.11 Hz, 1H), 7.78 (d, J=8.11 Hz, 1H), 7.44-7.53 (m, 3H), 5.15 (d, J=6.32 Hz, 2H), 2.61 (s, 3H).

### Example 336

### 1-(6-ethyl-2-methylpyridin-3-yl)-N-((5-phenyl-1,3,4-thiadiazol-2-yl)methyl)-1H-1,2,3-triazole-4-carboxamide

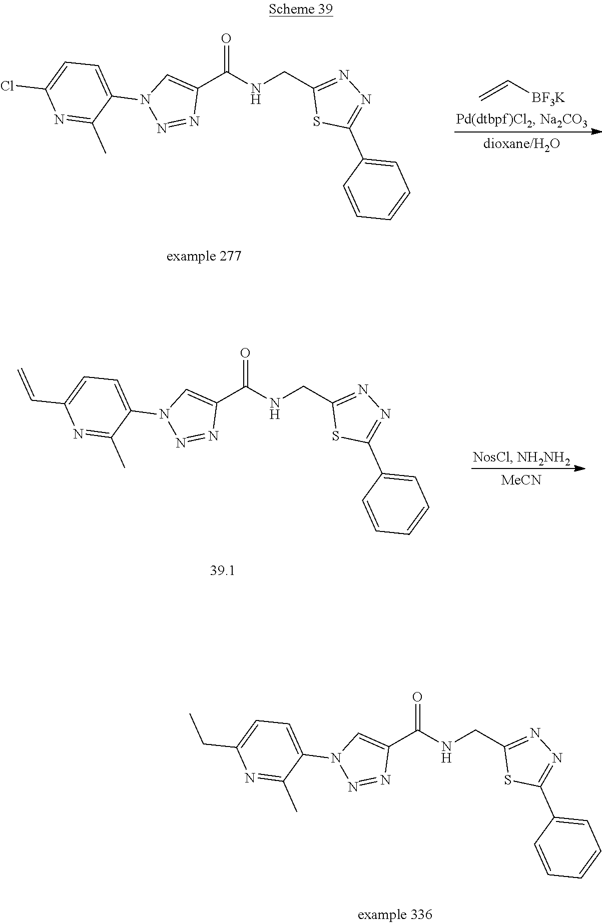

### Step 1: Preparation of 1-(2-methyl-6-vinylpyridin-3-yl)-N-((5-phenyl-1,3,4-thiadiazol-2-yl)methyl)-1H-1,2,3-triazole-4-carboxamide

A mixture of 1-(6-chloro-2-methylpyridin-3-yl)-N-((5-phenyl-1,3,4-thiadiazol-2-yl)methyl)-1H-1,2,3-triazole-4-carboxamide (65 mg, 0.158 mmol), potassium vinyltrifluoroborate (31.7 mg, 0.237 mmol), Na₂CO₃ (50.2 mg, 0.473 mmol) and PdCl₂(dtbpf) (10.29 mg, 0.016 mmol) in 1,4-Dioxane (0.9 mL) and water (0.1 mL) was degassed and backfilled with N₂ (three times). The mixture was heated to 100° C. for 1 hour. After cooling to room temperature the mixture was purified by silica gel chromatography (eluting ethyl acetate in petroleum ether gradient) to afford 1-(2-methyl-6-vinylpyridin-3-yl)-N-((5-phenyl-1,3,4-thiadiazol-2-yl)methyl)-1H-1,2,3-triazole-4-carboxamide. LCMS (ESI) m/z: 404 (M+H)⁺.

### Step 2: preparation of 1-(6-ethyl-2-methylpyridin-3-yl)-N-((5-phenyl-1,3,4-thiadiazol-2-yl)methyl)-1H-1,2,3-triazole-4-carboxamide

Hydrazinium hydroxide (13.43 mg, 0.228 mmol) was added to a solution of 1-(2-methyl-6-vinylpyridin-3-yl)-N-((5-phenyl-1,3,4-thiadiazol-2-yl)methyl)-1H-1,2,3-triazole-4-carboxamide (23 mg, 0.057 mmol) and 2-nitrobenzenesulfonyl chloride (25.3 mg, 0.114 mmol) in MeCN (1 mL) at 0° C. over 1 minute. After stirring for 10 minutes at 0° C., the reaction was warmed to 25° C. and stirred vigorously for 16 hours. The mixture was purified via reverse phase HPLC (eluting acetonitrile in water with 0.05% NH₄OH+10 mM NH₄HCO₃) to afford 1-(6-ethyl-2-methylpyridin-3-yl)-N-((5-phenyl-1,3,4-thiadiazol-2-yl)methyl)-1H-1,2,3-triazole-4-carboxamide. LCMS (ESI) m/z: 406 [M+H]⁺. ¹H NMR (400 MHz, CDCl₃) δ 8.33 (s, 1H), 7.90-8.02 (m, 3H), 7.60 (d, J=8.11 Hz, 1H), 7.44-7.52 (m, 3H), 7.22 (d, J=8.34 Hz, 1H), 5.14 (d, J=6.20 Hz, 2H), 2.88-2.95 (m, 2H), 2.45 (s, 3H), 1.36 (t, J=7.63 Hz, 3H)

### Example 337

### Preparation of 1-(2-cyano-6-methylpyridin-3-yl)-N-((5-phenyl-1,3,4-thiadiazol-2-yl)methyl)-1H-1,2,3-triazole-4-carboxamide

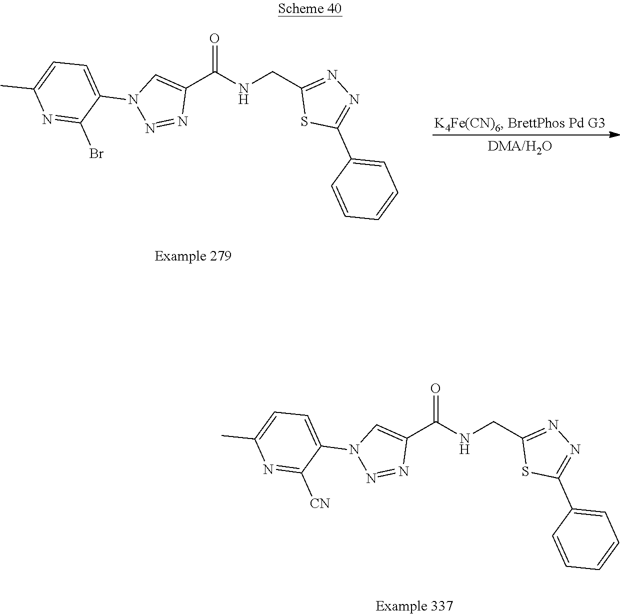 [SMILES: Cc1ccc(-n2cc(C(=O)NCc3nnc(-c4ccccc4)s3)nn2)c(Br)n1 ; Cc1ccc(-n2cc(C(=O)NCc3nnc(-c4ccccc4)s3)nn2)c(C#N)n1]

A mixture of 1-(2-bromo-6-methylpyridin-3-yl)-N-((5-phenyl-1,3,4-thiadiazol-2-yl)methyl)-1H-1,2,3-triazole-4-carboxamide (300 mg, 0.657 mmol), potassium hexacyanoferrate(II) trihydrate (139 mg, 0.329 mmol) and BrettPhos Pd G3 (59.6 mg, 0.066 mmol) in DMA (5 mL) and water (0.2 mL) was stirred at 100° C. for 18 hours. The mixture was filtered and the filtrate was purified via reverse phase HPLC (eluting acetonitrile in water with 0.1% NH₄OH) to afford 1-(2-cyano-6-methylpyridin-3-yl)-N-((5-phenyl-1,3,4-thiadiazol-2-yl)methyl)-1H-1,2,3-triazole-4-carboxamide. LCMS (ESI) m/z: 403 (M+H)⁺. ¹H NMR (CDCl₃, 400 MHz) δ 8.79 (s, 1H), 8.09 (d, J=8.46 Hz, 1H), 7.99-8.06 (m, 1H), 7.90-7.98 (m, 2H), 7.62 (d, J=8.46 Hz, 1H), 7.43-7.52 (m, 3H), 5.15 (d, J=6.32 Hz, 2H), 2.74 (s, 3H).

### Example 338

### Preparation of 1-(2-(2-hydroxypropan-2-yl)-6-methylpyridin-3-yl)-N-((5-phenyl-1,3,4-thiadiazol-2-yl)methyl)-1H-1,2,3-triazole-4-carboxamide

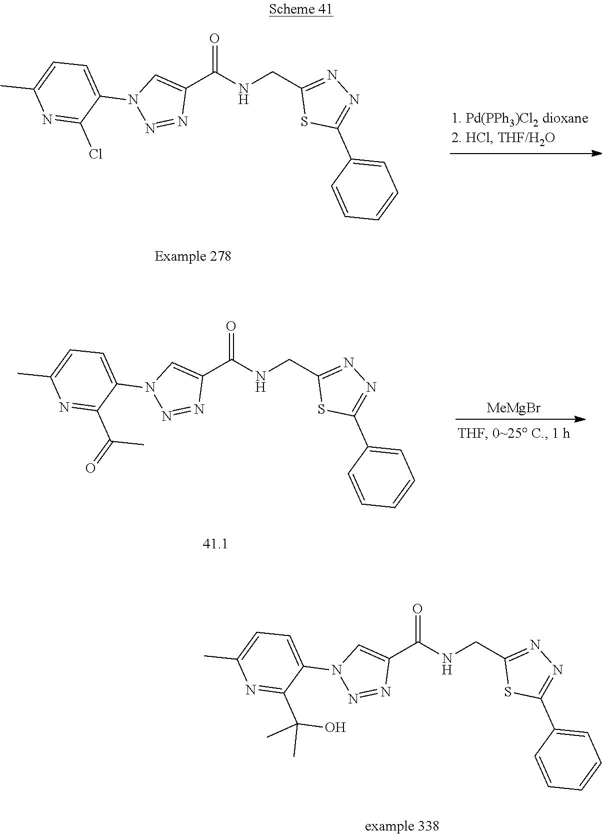

### Step 1. Preparation of 1-(2-acetyl-6-methylpyridin-3-yl)-N-((5-phenyl-1,3,4-thiadiazol-2-yl)methyl)-1H-1,2,3-triazole-4-carboxamide

A mixture of 1-(2-chloro-6-methylpyridin-3-yl)-N-((5-phenyl-1,3,4-thiadiazol-2-yl)methyl)-1H-1,2,3-triazole-4-carboxamide (50 mg, 0.121 mmol), Pd(PPh₃)₂Cl₂ (3.41 mg, 4.86 μmol) and tributyl(1-ethoxyvinyl)stannane (0.047 mL, 0.138 mmol) in dioxane (2 mL) was stirred at 100° C. for 1 hour. After cooling to room temperature, HCl (0.020 mL, 0.121 mmol) was added and the mixture was left to stir for 1 hour. The reaction mixture was quenched with saturated aqueous KF solution (10 mL). The mixture was filtered and the filtrate was extracted with EtOAc (3×10 mL). The combined organic phases were washed with brine (20 mL), dried over anhydrous Na₂SO₄, filtered and concentrated under reduced pressure. The residue was purified by silica gel chromatography (eluting ethyl acetate in petroleum ether) to afford 1-(2-acetyl-6-methylpyridin-3-yl)-N-((5-phenyl-1,3,4-thiadiazol-2-yl)methyl)-1H-1,2,3-triazole-4-carboxamide. LCMS (ESI) m/z: 420 (M+H)⁺.

### Step 2. Preparation of 1-(2-(2-hydroxypropan-2-yl)-6-methylpyridin-3-yl)-N-((5-phenyl-1,3,4-thiadiazol-2-yl)methyl)-1H-1,2,3-triazole-4-carboxamide

A mixture of 1-(2-acetyl-6-methylpyridin-3-yl)-N-((5-phenyl-1,3,4-thiadiazol-2-yl)methyl)-1H-1,2,3-triazole-4-carboxamide (40 mg, 0.095 mmol), methylmagnesium bromide solution (0.064 mL, 0.191 mmol) in THF (1 mL) was stirred at 0° C. for 1 hour. The reaction mixture was quenched with saturated NH₄Cl (2 mL) and the mixture was filtered and the filtrate was extracted with EtOAc (3×5 mL). The combined organic phases were washed with brine (20 mL), dried over anhydrous Na₂SO₄, filtered and concentrated under reduced pressure. The residue was purified via reverse phase HPLC (eluting acetonitrile in water with 0.1% NH₄OH) to afford 1-(2-(2-hydroxypropan-2-yl)-6-methylpyridin-3-yl)-N-((5-phenyl-1,3,4-thiadiazol-2-yl)methyl)-1H-1,2,3-triazole-4-carboxamide. LCMS (ESI) m/z: 436 (M+H)⁺. ¹H NMR (CDCl₃, 400 MHz) δ 8.29 (s, 1H), 7.88-8.08 (m, 3H), 7.44-7.51 (m, 4H), 7.28 (d, J=7.99 Hz, 1H), 5.13 (d, J=6.32 Hz, 2H), 2.69 (s, 3H), 1.28 (s, 6H).

### Example 339

### Preparation of 1-(6-methyl-2-oxo-1,2-dihydropyridin-3-yl)-N-((5-phenyl-1,3,4-thiadiazol-2-yl)methyl)-1H-1,2,3-triazole-4-carboxamide

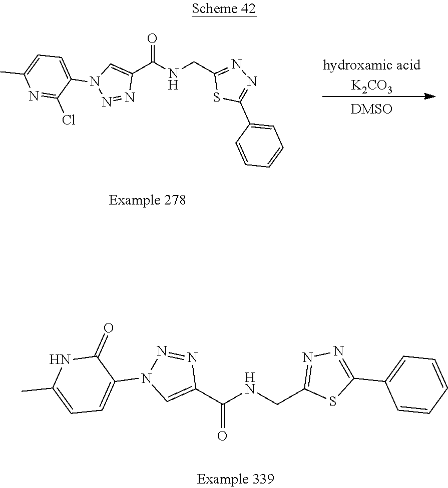 [SMILES: Cc1ccc(-n2cc(C(=O)NCc3nnc(-c4ccccc4)s3)nn2)c(Cl)n1 ; Cc1ccc(-n2cc(C(=O)NCc3nnc(-c4ccccc4)s3)nn2)c(=O)[nH]1]

A mixture of 1-(2-chloro-6-methylpyridin-3-yl)-N-((5-phenyl-1,3,4-thiadiazol-2-yl)methyl)-1H-1,2,3-triazole-4-carboxamide (50 mg, 0.121 mmol), acetohydroxamic acid (27.3 mg, 0.364 mmol) and K₂CO₃ (84 mg, 0.607 mmol) in DMSO (1 mL) was stirred for 16 hours at 60° C., The mixture was quenched with water (5 mL) and washed with brine (10 mL), then filtered to give 1-(6-methyl-2-oxo-1,2-dihydropyridin-3-yl)-N-((5-phenyl-1,3,4-thiadiazol-2-yl)methyl)-1H-1,2,3-triazole-4-carboxamide. LCMS (ESI) m/z: 394 (M+H)⁺)⁺. ¹H NMR (DMSO, 400 MHz) δ 12.49-12.64 (m, 1H), 9.66-9.72 (m, 1H), 9.15 (s, 1H), 8.08 (d, J=7.51 Hz, 1H), 7.94-8.01 (m, 2H), 7.49-7.59 (m, 3H), 6.29 (d, J=7.75 Hz, 1H), 4.87-4.96 (m, 2H), 2.31 (s, 3H).

### Example 340

### Preparation of 1-(2-amino-6-methylpyridin-3-yl)-N-((5-phenyl-1,3,4-thiadiazol-2-yl)methyl)-1H-1,2,3-triazole-4-carboxamide

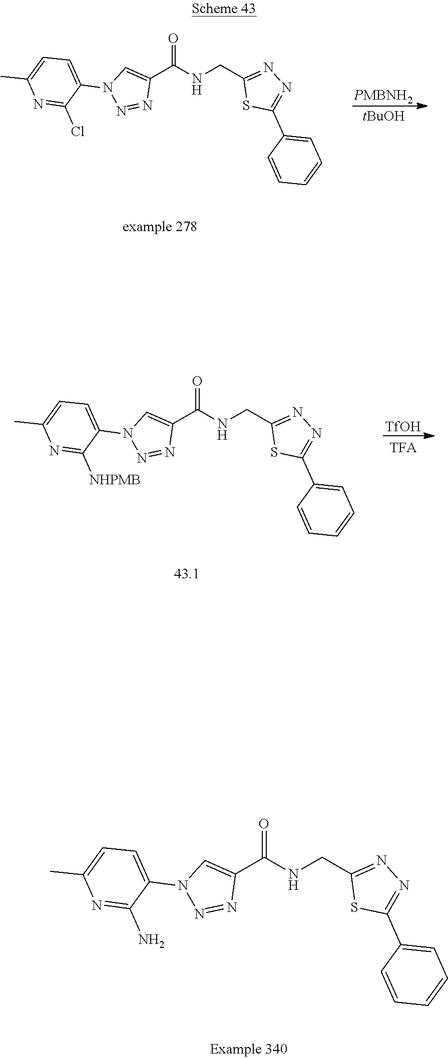

### Step 1. Preparation of 1-(2-((4-methoxybenzyl)amino)-6-methylpyridin-3-yl)-N-((5-phenyl-1,3,4-thiadiazol-2-yl)methyl)-1H-1,2,3-triazole-4-carboxamide

A mixture of (4-methoxyphenyl)methanamine (33.3 mg, 0.243 mmol), 1-(2-chloro-6-methylpyridin-3-yl)-N-((5-phenyl-1,3,4-thiadiazol-2-yl)methyl)-1H-1,2,3-triazole-4-carboxamide (50 mg, 0.121 mmol), DIEA (0.064 mL, 0.364 mmol) in tBuOH (1 mL) was stirred for 72 hours at 120° C. The mixture was concentrated under reduced pressure to afford the title compound which was used without further purification. LCMS (ESI) m/z: 513 (M+H)⁺.

### Step 2: Preparation of 1-(2-amino-6-methylpyridin-3-yl)-N-((5-phenyl-1,3,4-thiadiazol-2-yl)methyl)-1H-1,2,3-triazole-4-carboxamide

A mixture of 1-(2-((4-methoxybenzyl)amino)-6-methylpyridin-3-yl)-N-((5-phenyl-1,3,4-thiadiazol-2-yl)methyl)-1H-1,2,3-triazole-4-carboxamide (50 mg, 0.098 mmol) in TFA (1 mL) and TfOH (0.2 mL) was stirred for 1 hour at 25° C. The reaction mixture was quenched with NH₄OH (5 mL) and extracted with DCM:MeOH (10:1, 3×20 mL). The combined organic phases were washed with brine (30 mL), dried over anhydrous Na₂SO₄, filtered and concentrated. The residue was washed with MeOH (10 mL) then concentrated and dried under vacuum to afford 1-(2-amino-6-methylpyridin-3-yl)-N-((5-phenyl-1,3,4-thiadiazol-2-yl)methyl)-1H-1,2,3-triazole-4-carboxamide. LCMS (ESI) m/z: 393 (M+H)⁺. ¹H NMR (DMSO, 400 MHz) δ 9.61-9.68 (m, 1H), 8.93 (s, 1H), 7.94-7.99 (m, 2H), 7.52-7.58 (m, 4H), 6.59 (d, J=7.87 Hz, 1H), 6.21 (s, 2H), 4.91 (d, J=6.08 Hz, 2H), 2.35 (s, 3H)

### Example 341

### Preparation of 1-(2-methyl-6-(methylsulfonyl)pyridin-3-yl)-N-((5-phenyl-1,3,4-thiadiazol-2-yl)methyl)-1H-1,2,3-triazole-4-carboxamide

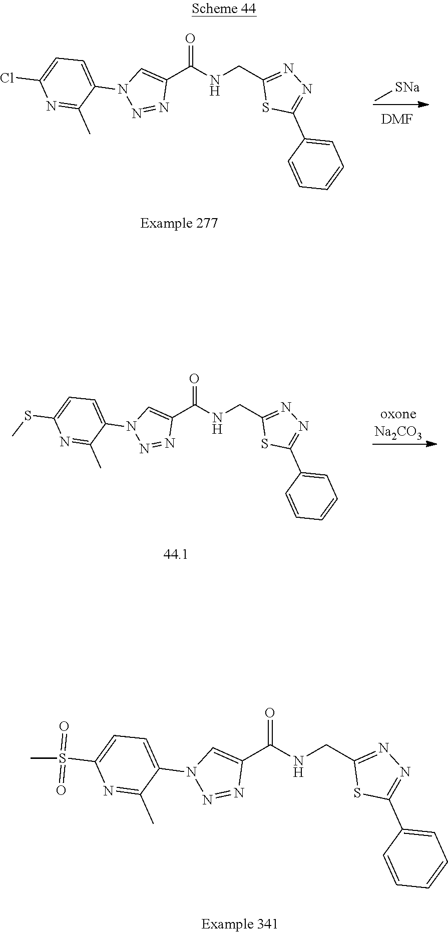

### Step 1. Preparation of 1-(2-methyl-6-(methylthio)pyridin-3-yl)-N-((5-phenyl-1,3,4-thiadiazol-2-yl)methyl)-1H-1,2,3-triazole-4-carboxamide

A mixture of 1-(6-chloro-2-methylpyridin-3-yl)-N-((5-phenyl-1,3,4-thiadiazol-2-yl)methyl)-1H-1,2,3-triazole-4-carboxamide (40 mg, 0.097 mmol) and MeSNa (13.61 mg, 0.194 mmol) in DMF (2 mL) was stirred at 70° C. for 1 hour. The reaction mixture was quenched with saturated aqueous NaHCO₃ (2 mL) and extracted with EtOAc (3×2 mL). The combined organic phases were washed with brine (2 mL), dried over anhydrous Na₂SO₄, filtered and concentrated under reduced pressure to afford 1-(2-methyl-6-(methylthio)pyridin-3-yl)-N-((5-phenyl-1,3,4-thiadiazol-2-yl)methyl)-1H-1,2,3-triazole-4-carboxamide. LCMS (ESI) m/z: 424 (M+H)⁺.

### Step 2. 1-(2-methyl-6-(methylsulfonyl)pyridin-3-yl)-N-((5-phenyl-1,3,4-thiadiazol-2-yl)methyl)-1H-1,2,3-triazole-4-carboxamide

Oxone (383 mg, 0.623 mmol) was added to a solution of 1-(2-methyl-6-(methylthio)pyridin-3-yl)-N-((5-phenyl-1,3,4-thiadiazol-2-yl)methyl)-1H-1,2,3-triazole-4-carboxamide (40 mg, 0.094 mmol) and Na₂CO₃ (130 mg, 1.228 mmol) in acetone (2 mL) and water (1.2 mL) at 0° C. over 3 minutes. After stirring for 1 hours at 25° C. the reaction mixture was quenched with saturated aqueous Na₂SO₃ (5 mL) and extracted with EtOAc (3×5 mL). The combined organic phases were washed with brine (5 mL), dried over anhydrous Na₂SO₄, filtered and concentrated under reduced pressure. The residue was purified via reverse phase HPLC (eluting acetonitrile in water with 0.05% NH₄OH+10 mM NH₄HCO₃ modifier) to afford the title compound. LCMS (ESI) m/z: 456 (M+H)⁺. ¹H NMR (400 MHz, CDCl₃) δ 8.44 (s, 1H), 8.13-8.19 (m, 1H), 8.02-8.08 (m, 1H), 7.98-8.02 (m, 1H), 7.92-7.97 (m, 2H), 7.45-7.53 (m, 3H), 5.14 (d, J=6.32 Hz, 2H), 3.31 (s, 3H), 2.64 (s, 3H)

### Example 342

### Preparation of 1-(6-amino-2-methylpyridin-3-yl)-N-((5-phenyl-1,3,4-thiadiazol-2-yl)methyl)-1H-1,2,3-triazole-4-carboxamide

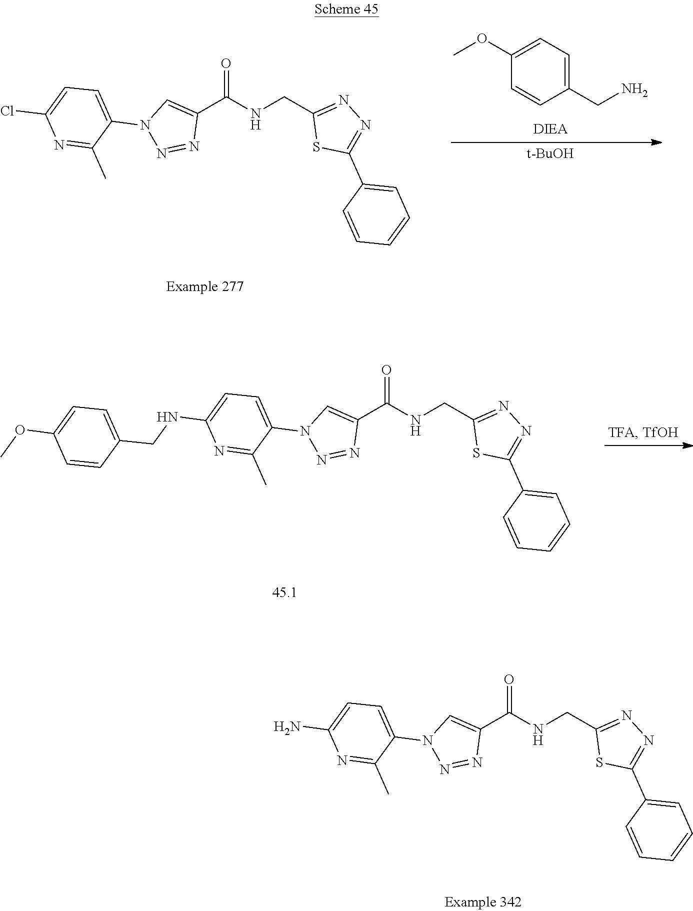

### Step 1. Preparation of 1-(6-((4-methoxybenzyl)amino)-2-methylpyridin-3-yl)-N-((5-phenyl-1,3,4-thiadiazol-2-yl)methyl)-1H-1,2,3-triazole-4-carboxamide

A mixture of 1-(6-chloro-2-methylpyridin-3-yl)-N-((5-phenyl-1,3,4-thiadiazol-2-yl)methyl)-1H-1,2,3-triazole-4-carboxamide (50 mg, 0.121 mmol), (4-methoxyphenyl)methanamine (0.032 mL, 0.243 mmol) and DIEA (0.064 mL, 0.364 mmol) in t-BuOH (1 mL) was stirred at 120° C. for 24 hours. The reaction mixture was quenched with water (2 mL) and extracted with EtOAc (3×2 mL). The combined organic phases were washed with brine (2 mL), dried over anhydrous Na₂SO₄, filtered and concentrated under reduced pressure to afford 1-(6-((4-methoxybenzyl)amino)-2-methylpyridin-3-yl)-N-((5-phenyl-1,3,4-thiadiazol-2-yl)methyl)-1H-1,2,3-triazole-4-carboxamide. LCMS: 513 (M+H)⁺.

### Step 2. Preparation of 1-(6-amino-2-methylpyridin-3-yl)-N-((5-phenyl-1,3,4-thiadiazol-2-yl)methyl)-1H-1,2,3-triazole-4-carboxamide

A mixture of 1-(6-((4-methoxybenzyl)amino)-2-methylpyridin-3-yl)-N-((5-phenyl-1,3,4-thiadiazol-2-yl)methyl)-1H-1,2,3-triazole-4-carboxamide (50 mg, 0.098 mmol) in TFA (1 mL) and TfOH (0.2 mL) was stirred for 0.5 hour at 25° C. The reaction mixture was quenched with NH₄OH (5 mL) and extracted with EtOAc (3×2 mL). The combined organic phases were washed with brine (2 mL), dried over anhydrous Na₂SO₄, filtered and concentrated under reduced pressure. The residue purified via reverse phase HPLC (eluting acetonitrile in water 0.05% NH₄OH+10 mM NH₄OH) to afford 1-(6-amino-2-methylpyridin-3-yl)-N-((5-phenyl-1,3,4-thiadiazol-2-yl)methyl)-1H-1,2,3-triazole-4-carboxamide. LCMS (ESI) m/z: 393 (M+H)⁺. ¹H NMR (400 MHz, DMSO-d₆) δ 9.65 (t, J=6.14 Hz, 1H), 8.91 (s, 1H), 7.93-8.00 (m, 2H), 7.51-7.58 (m, 3H), 7.44 (d, J=8.58 Hz, 1H), 6.46 (s, 2H), 6.40 (d, J=8.58 Hz, 1H), 4.88-4.92 (m, 2H), 2.07 (s, 3H).

### Example 343

### Preparation of 1-((1-(hydroxymethyl)cyclopropyl)methyl)-N-((5-phenyl-1,3,4-thiadiazol-2-yl)methyl)-1H-1,2,3-triazole-4-carboxamide

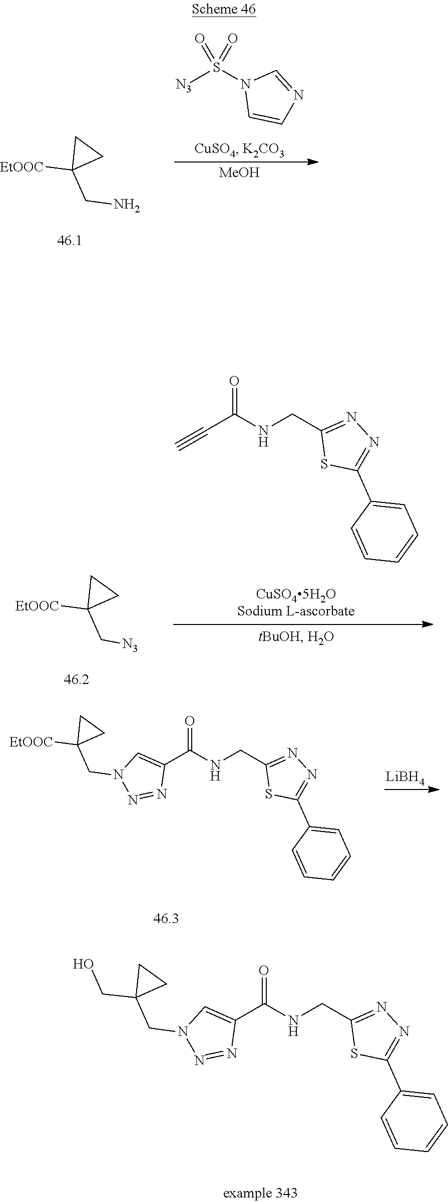

### Step 1. Preparation of ethyl 1-(azidomethyl)cyclopropane-1-carboxylate

A mixture of ethyl 1-(aminomethyl)cyclopropanecarboxylate (200 mg, 1.397 mmol), 1H-imidazole-1-sulfonyl azide hydrochloride (293 mg, 1.397 mmol), K₂CO₃ (290 mg, 2.095 mmol) and copper(II) sulfate (11.15 mg, 0.070 mmol) in MeOH (5 mL) was stirred for 2 hours at 25° C. The reaction mixture was quenched with water (10 mL) and extracted with EtOAc (3×10 mL). The combined organic phases were washed with brine (10 mL), dried over anhydrous Na₂SO₄, filtered and concentrated under reduced pressure to afford ethyl 1-(azidomethyl)cyclopropane-1-carboxylate, which was used in the next step directly without further purification.

### Step 2. Preparation of ethyl 1-((4-(((5-phenyl-1,3,4-thiadiazol-2-yl)methyl)carbamoyl)-1H-1,2,3-triazol-1-yl)methyl)cyclopropane-1-carboxylate

A mixture of ethyl 1-(azidomethyl)cyclopropane-1-carboxylate (100 mg, 0.591 mmol), N-((5-phenyl-1,3,4-thiadiazol-2-yl)methyl)propiolamide (158 mg, 0.650 mmol), copper(II) sulfate (9.43 mg, 0.059 mmol) and (+)-sodium L-ascorbate (11.71 mg, 0.059 mmol) in t-BuOH (1 mL) was stirring for 16 hours at 50° C. The reaction mixture was quenched with water (2 mL) and extracted with EtOAc (3×2 mL). The combined organic phases were washed with brine (2 mL), dried over anhydrous Na₂SO₄, filtered and concentrated under reduced pressure. The residue was purified by silica gel chromatography (eluting ethyl acetate in petroleum ether) to afford ethyl 1-((4-(((5-phenyl-1,3,4-thiadiazol-2-yl)methyl)carbamoyl)-1H-1,2,3-triazol-1-yl)methyl)cyclopropane-1-carboxylate. LCMS (ESI) m/z: 413 (M+H)⁺.

### Step 3. Preparation of 1-(5-chloro-2-(difluoromethoxy)benzyl)-4-(thiazol-5-yl)pyrimidin-2(1H)-one

LiBH₄ (2.72 mg, 0.125 mmol) was added to a solution of ethyl 1-((4-(((5-phenyl-1,3,4-thiadiazol-2-yl)methyl)carbamoyl)-1H-1,2,3-triazol-1-yl)methyl)cyclopropane-1-carboxylate (40 mg, 0.083 mmol) in THF (1 mL) at 0° C. over 3 minutes. After stirring for 16 hours at 25° C. the reaction mixture was quenched with water (2 mL) and extracted with EtOAc (3×2 mL). The combined organic phases were washed with brine (2 mL), dried over anhydrous Na₂SO₄, filtered and concentrated. The residue was purified via reverse phase HPLC (eluting acetonitrile in water with 0.05% NH₄OH+10 mM NH₄HCO₃) to afford 1-((1-(hydroxymethyl)cyclopropyl)methyl)-N-((5-phenyl-1,3,4-thiadiazol-2-yl)methyl)-1H-1,2,3-triazole-4-carboxamide. LCMS (ESI) m/z: 371 (M+H)⁺. ¹H NMR (400 MHz, DMSO) δ 9.55 (t, J=6.14 Hz, 1H), 8.62 (s, 1H), 7.93-7.99 (m, 2H), 7.50-7.57 (m, 3H), 4.83-4.90 (m, 2H), 4.77 (t, J=5.42 Hz, 1H), 4.41 (s, 2H), 3.10-3.16 (m, 2H), 0.65-0.72 (m, 2H), 0.48-0.54 (m, 2H)

### Example 344 & 345

### Preparation of N-((6-(2H-1,2,3-triazol-2-yl)pyridazin-3-yl)methyl)-1-(2,6-dimethylpyridin-3-yl)-1H-1,2,3-triazole-4-carboxamide, 344, & 345, N-((6-(1H-1,2,3-triazol-1-yl)pyridazin-3-yl)methyl)-1-(2,6-dimethylpyridin-3-yl)-1H-1,2,3-triazole-4-carboxamide

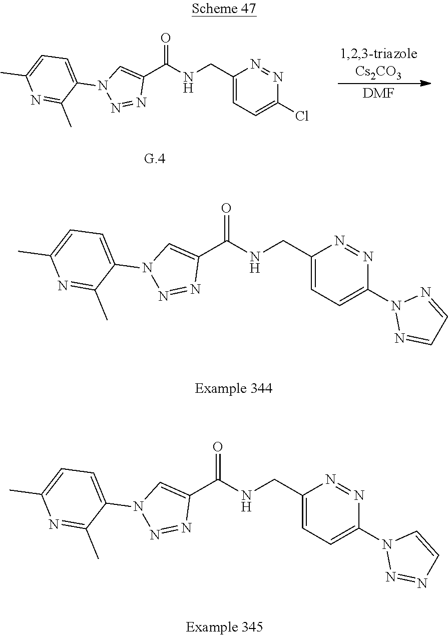

A mixture of N-((6-chloropyridazin-3-yl)methyl)-1-(2,6-dimethylpyridin-3-yl)-1H-1,2,3-triazole-4-carboxamide (99 mg, 0.288 mmol), 1H-1,2,3-triazole (0.050 mL, 0.864 mmol) and Cs₂CO₃ (281 mg, 0.864 mmol) in DMF (2 mL) was stirred at 110° C. for 16 hours. The mixture was filtered and the filtrate was purified via reverse phase HPLC (eluting acetonitrile in water with 0.1% TFA) to afford a mixture of N-((6-(2H-1,2,3-triazol-2-yl)pyridazin-3-yl)methyl)-1-(2,6-dimethylpyridin-3-yl)-1H-1,2,3-triazole-4-carboxamide and N-((6-(1H-1,2,3-triazol-1-yl)pyridazin-3-yl)methyl)-1-(2,6 dimethylpyridin-3-yl)-1H-1,2,3-triazole-4-carboxamide.

The products were separated by Chiral-SFC (DAICEL CHIRALPAK AD, 250 mm×30 mm, 40% EtOH, 0.05% DEA co-solvent)

### Example 344, Peak 1

LCMS (ESI) m/z: 377 (M+H)⁺. ¹H NMR (CDCl₃, 400 MHz) δ 8.23-8.34 (m, 2H), 8.15-8.22 (m, 1H), 8.01 (s, 2H), 7.82-7.94 (m, 1H), 7.59 (d, J=8.11 Hz, 1H), 7.22 (d, J=8.11 Hz, 1H), 5.11 (d, J=6.08 Hz, 2H), 2.66 (s, 3H), 2.45 (s, 3H)

### Example 345, Peak 2

LCMS (ESI) m/z: 377 (M+H)⁺. ¹H NMR (CDCl₃, 400 MHz) δ 8.82-8.86 (m, 1H), 8.46 (d, J=9.06 Hz, 1H), 8.32 (s, 1H), 8.13-8.20 (m, 1H), 7.84-7.97 (m, 2H), 7.59 (d, J=8.11 Hz, 1H), 7.22 (d, J=8.11 Hz, 1H), 5.10 (d, J=6.08 Hz, 2H), 2.67 (s, 3H), 2.46 (s, 3H)

### Example 346

### Preparation of 1-(3,5-dimethyl-1H-pyrazol-4-yl)-N-((5-phenyl-1,3,4-thiadiazol-2-yl)methyl)-1H-1,2,3-triazole-4-carboxamide

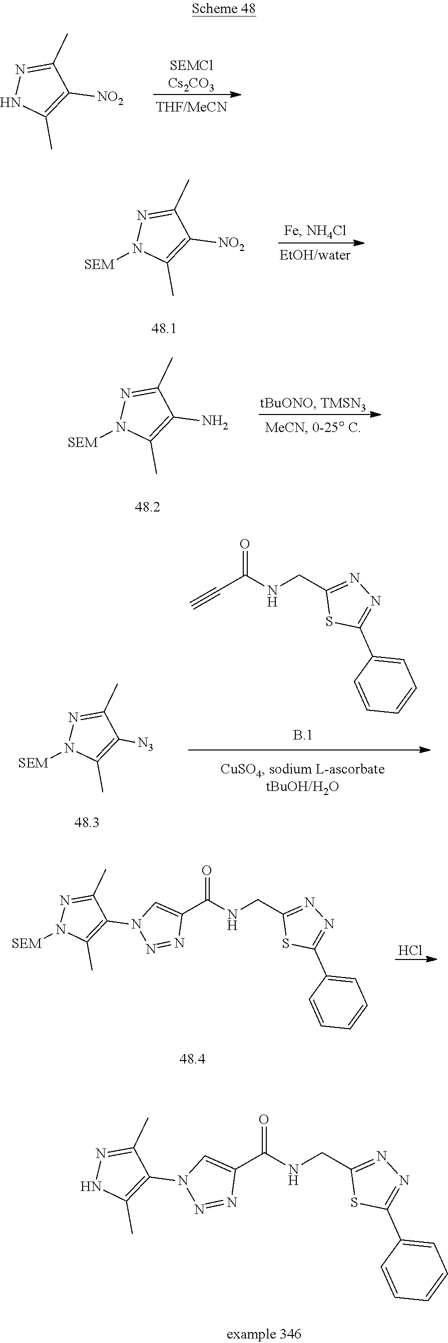

### Step 1. Preparation of 3,5-dimethyl-4-nitro-1-((2-(trimethylsilyl)ethoxy)methyl)-1H-pyrazole

A mixture of 3,5-dimethyl-4-nitro-1H-pyrazole (1 g, 7.09 mmol), Cs₂CO₃ (4.62 g, 14.17 mmol) and SEM-Cl (1.885 mL, 10.63 mmol) in THF (12 mL) and MeCN (8.00 mL) was stirred at 25° C. for 16 hours. The mixture was filtered and the filtrate was concentrated under reduced pressure. The residue was dissolved in water (100 mL) and EtOAc (100 mL). The organic layer was separated and the aqueous was extracted with EtOAc (3×50 mL) and the combined organic layers were washed with brine (200 mL), dried over anhydrous Na₂SO₄, filtered and concentrated under reduced pressure. The residue was purified by silica gel chromatography (eluting with ethyl acetate in petroleum ether) to afford 3,5-dimethyl-4-nitro-1-((2-(trimethylsilyl)ethoxy)methyl)-1H-pyrazole. LCMS (ESI) m/z: 272 (M+H)⁺.

### Step 2. Preparation of 3,5-dimethyl-1-((2-(trimethylsilyl)ethoxy)methyl)-1H-pyrazol-4-amine

A mixture of 3,5-dimethyl-4-nitro-1-((2-(trimethylsilyl)ethoxy)methyl)-1H-pyrazole (1.2 g, 4.42 mmol), iron (1.235 g, 22.11 mmol) and ammonium chloride (1.183 g, 22.11 mmol) in ethanol (15 mL) and Water (3.75 mL) was stirred at 80° C. for 2 hours. The mixture was filtered and the filtrate was concentrated under reduced pressure to afford 3,5-dimethyl-1-((2-(trimethylsilyl)ethoxy)methyl)-1H-pyrazol-4-amine which was used in the next step without further purification. LCMS (ESI) m/z: 242 (M+H)⁺.

### Step 3. Preparation of 4-azido-3,5-dimethyl-1-((2-(trimethylsilyl)ethoxy)methyl)-1H-pyrazole

Tert-butyl nitrite (0.074 mL, 0.621 mmol) was added to a solution of 3,5-dimethyl-1-((2-(trimethylsilyl)ethoxy)methyl)-1H-pyrazol-4-amine (100 mg, 0.414 mmol) in MeCN (1 mL) at 0° C. over 1 minutes, then TMS-N₃ (0.088 mL, 0.663 mmol) was added to the mixture at 0° C. The resulting mixture was stirred for another 16 hours. The reaction mixture was quenched with water (20 mL) and extracted with EtOAc (3×20 mL). The combined organic phases were washed with brine (50 mL), dried over anhydrous Na₂SO₄, filtered and concentrated under reduced pressure to afford 4-azido-3,5-dimethyl-1-((2-(trimethylsilyl)ethoxy)methyl)-1H-pyrazole which was used in the next step without further purification. LCMS (ESI) m/z: 268 (M+H)⁺.

### Step 4. Preparation of 1-(3,5-dimethyl-1-((2-(trimethylsilyl)ethoxy)methyl)-1H-pyrazol-4-yl)-N-((5-phenyl-1,3,4-thiadiazol-2-yl)methyl)-1H-1,2,3-triazole-4-carboxamide

CuSO₄ (5.97 mg, 0.037 mmol) and a solution of (+)-sodium L-ascorbate (14.82 mg, 0.075 mmol) in water (0.6 mL) were added to a solution of 4-azido-3,5-dimethyl-1-((2-(trimethylsilyl)ethoxy)methyl)-1H-pyrazole (100 mg, 0.374 mmol) and N-((5-phenyl-1,3,4-thiadiazol-2-yl)methyl)propiolamide (100 mg, 0.411 mmol) in t-BuOH (3 mL) at 25° C. over 2 minutes. After stirring for 16 hours at 50° C. the mixture was cooled to room temperature, filtered and the filtrate was concentrated under reduced pressure. The residue was purified via reverse phase HPLC (eluting acetonitrile in water with 0.1% TFA modifier) to afford 1-(3,5-dimethyl-1-((2-(trimethylsilyl)ethoxy)methyl)-1H-pyrazol-4-yl)-N-((5-phenyl-1,3,4-thiadiazol-2-yl)methyl)-1H-1,2,3-triazole-4-carboxamide. LCMS (ESI) m/z: 511 (M+H)⁺.

### Step 5. Preparation of 1-(3,5-dimethyl-1H-pyrazol-4-yl)-N-((5-phenyl-1,3,4-thiadiazol-2-yl)methyl)-1H-1,2,3-triazole-4-carboxamide

A mixture of 1-(3,5-dimethyl-1-((2-(trimethylsilyl)ethoxy)methyl)-1H-pyrazol-4-yl)-N-((5-phenyl-1,3,4-thiadiazol-2-yl)methyl)-1H-1,2,3-triazole-4-carboxamide (60 mg, 0.117 mmol) and HCl in MeOH (2 mL, 8.00 mmol) in DCM (2 mL) was stirred at 25° C. for 16 hours. The solvent was removed under reduced pressure and the residue was purified via reverse phase HPLC (eluting acetonitrile in water with 0.05% NH₄OH+10 mM NH₄HCO₃) to afford 1-(3,5-dimethyl-1H-pyrazol-4-yl)-N-((5-phenyl-1,3,4-thiadiazol-2-yl)methyl)-1H-1,2,3-triazole-4-carboxamide.

LCMS (ESI) m/z: 381 (M+H)⁺. ¹H NMR (400 MHz, DMSO-d₆) δ 12.82 (br s, 1H), 9.64 (t, J=6.02 Hz, 1H), 8.92 (s, 1H), 7.94-7.99 (m, 2H), 7.51-7.58 (m, 3H), 4.90 (d, J=6.08 Hz, 2H), 2.15 (s, 6H).

### Example 347

### Preparation of 1-(2,2-difluoroethyl)-N-((5-(pyridin-4-yl)-1,3,4-thiadiazol-2-yl)methyl)-1H-1,2,3-triazole-4-carboxamide

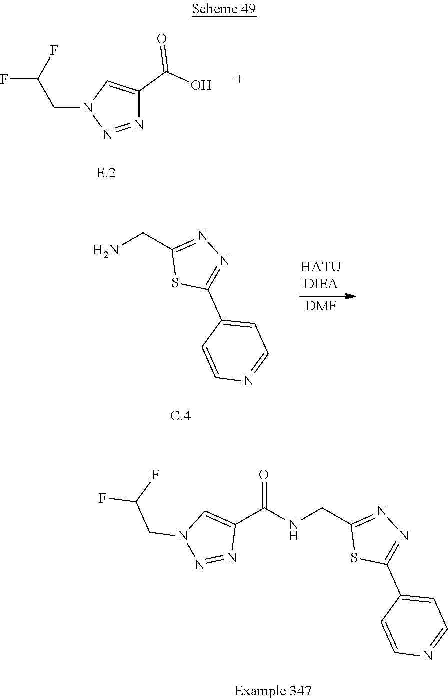

HATU (97 mg, 0.254 mmol) and DIEA (0.118 mL, 0.678 mmol) were added to a solution of 1-(2,2-difluoroethyl)-1H-1,2,3-triazole-4-carboxylic acid (30 mg, 0.169 mmol), in DMF (1 mL) at 25° C. over 2 minutes. After stirring for 5 minutes at 25° C., (5-(pyridin-4-yl)-1,3,4-thiadiazol-2-yl)methanamine hydrochloride (51.1 mg, 0.169 mmol) was added to the mixture at 25° C. and the resulting mixture was stirred for another 3 hours. The mixture was filtered and the filtrate was purified via reverse phase HPLC (eluting acetonitrile in water with 0.05% NH₄OH+10 mM NH₄HCO₃ modifier) to afford 1-(2,2-difluoroethyl)-N-((5-(pyridin-4-yl)-1,3,4-thiadiazol-2-yl)methyl)-1H-1,2,3-triazole-4-carboxamide. LCMS (ESI) m/z: 352 (M+H)⁺. ¹H NMR (400 MHz, DMSO-d₆) δ 9.67 (t, J=6.02 Hz, 1H), 8.74 (d, J=5.84 Hz, 2H), 8.70 (s, 1H), 7.91-7.95 (m, 2H), 6.38-6.69 (m, 1H), 5.04 (dt, J=3.04, 15.65 Hz, 2H), 4.91 (d, J=5.96 Hz, 2H)

### Example 348 & 349

### Preparation of 1-(2,2-difluoroethyl)-N-(1-(6-phenylpyridazin-3-yl)ethyl)-1H-1,2,3-triazole-4-carboxamide & 1-(2,2-difluoroethyl)-N-(1-(6-phenylpyridazin-3-yl)ethyl)-1H-1,2,3-triazole-4-carboxamide

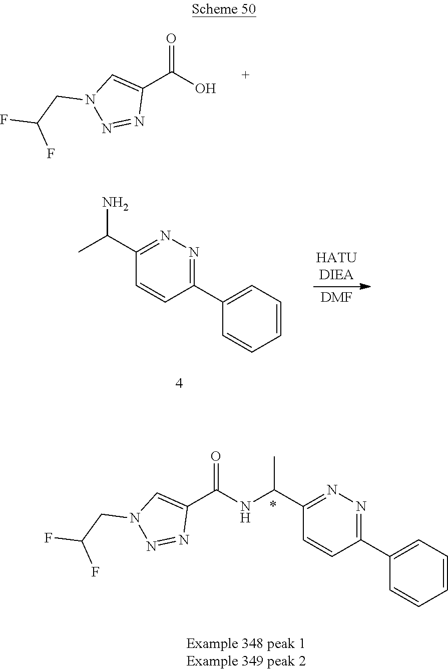

HATU (192 mg, 0.504 mmol) and DIEA (0.235 mL, 1.345 mmol) were added to a solution of 1-(2,2-difluoroethyl)-1H-1,2,3-triazole-4-carboxylic acid (59.6 mg, 0.336 mmol) in DMF (1 mL) at 25° C. over 1 minute. After stirring for 10 minutes at 25° C., 1-(6-phenylpyridazin-3-yl)ethan-1-amine (67 mg, 0.336 mmol) was added to the mixture at 25° C. The resulting mixture was stirred for another 1 hour at 25° C. The mixture was filtered and the filtrate was purified via reverse phase HPLC (eluting acetonitrile in water with 0.1% TFA modifier) to afford 1-(2,2-difluoroethyl)-N-(1-(6-phenylpyridazin-3-yl)ethyl)-1H-1,2,3-triazole-4-carboxamide. LCMS (ESI) m/z: 359 (M+H)⁺. The racemic mixture was resolved by chiral SFC purification (Cellulose-2 100×4.6 mm column, 40% EtOH w/0.05% DEA as cosolvent) to afford two peaks.

### Example 348 Peak 1

LCMS (ESI) m/z: 359 (M+H)⁺. ¹H NMR (CDCl₃, 400 MHz) δ 8.11-8.18 (m, 2H), 8.01 (dd, J=1.55, 7.87 Hz, 2H), 7.78 (d, J=8.82 Hz, 1H), 7.52 (d, J=8.82 Hz, 1H), 7.42-7.49 (m, 3H), 6.08 (tt, J=3.84, 54.63 Hz, 1H), 5.50 (quin, J=7.12 Hz, 1H), 4.73 (dt, J=3.81, 13.65 Hz, 2H), 1.72 (d, J=7.03 Hz, 3H)

### Example 349 Peak 2

LCMS (ESI) m/z: 359 (M+H)⁺. ¹H NMR (CDCl₃, 400 MHz) δ 8.11-8.18 (m, 2H), 8.01 (dd, J=1.61, 7.81 Hz, 2H), 7.78 (d, J=8.70 Hz, 1H), 7.52 (d, J=8.70 Hz, 1H), 7.43-7.49 (m, 3H), 6.08 (tt, J=3.87, 54.60 Hz, 1H), 5.50 (quin, J=7.18 Hz, 1H), 4.73 (dt, J=3.81, 13.65 Hz, 2H), 1.72 (d, J=6.91 Hz, 3H).

### Assay

### IL4I1 Enzymatic Assay

Interleukin 4 inducible protein 1 (IL4I1) is an L-amino oxidase that catalyzes the oxidation of aromatic residues (Phe, Trp and Tyr): L-amino acid+H₂O+O₂→2-oxo acid+NH₃+H₂O₂. Equal molar of H₂O₂ and the corresponding alpha-ketoacid are produced when IL4I1 and substrate are added. In this assay, the hydrogen peroxide generated by IL4I1 is then detected through a coupled reaction with Amplex Red (10-acetyl-3,7-dihydroxyphenoxazine) and Horse Peroxidase (HRP) to produce Resorufin product that could be detected in the form of fluorescence signals. The assessment of the inhibitory effect of small molecules (EC₅₀) on IL4I1 is measured by the effectiveness of the compounds to inhibit the production of H₂02.

Using this assay, the potency (EC₅₀) of each compound was determined from a ten-point (1:3 serial dilution) titration curve using the following outlined procedure. To each well of a black flat-bottom Greiner (Cat #781076) 384 well-plate, 125 nL of compound (0.5% DMSO in final assay volume of 25 μL) was dispensed, followed by the addition of 12.5 μL of 1× assay buffer (50 mM Hepes 7.0 and 0.005% Tween20 (Sigma, Cat #P8341; low peroxide grade)) containing 2 nM of recombinant IL4I1 (R&D Systems, Cat #5684-AO-020). Plates were placed in an ambient temperature humidified chamber for a four-hour pre-incubation with compound. Subsequently, each reaction was initiated by the addition of 12.5 μL 1× assay buffer containing 2 mM of each aromatic amino acids (Phe/Tyr/Trp), 0.1 mM Amplex Red and 2 U/mL of HRP. The final reaction in each well of 25 μL consists of 1 nM of IL4I1, 1 mM of each residue (Phe, Tyr and Trp), 0.05 mM Amplex Red and 1 U/mL of HRP. It should be noted that the concentrations of Amplex Red and HRP used here are in excess such that the conversion of H₂O₂ to Resorufin product occurs instantaneously and non-rate limiting. Reactions were allowed to proceed for 120 minutes followed by fluorescence readout on a Spectramax with the following set parameters: 544 nm excitation/590 nm emission, 570 nm cutoff (EnVision is an alternative reader). Dose-response curves were generated by plotting percent effect (% inhibition; Y-axis) vs. Log₁₀ compound concentrations (X-axis). EC₅₀ values were calculated using a non-linear regression, four-parameters sigmoidal dose-response model and are shown in Table 16.

Potency Table 16:

| Example No. | IL4I1 EC₅₀ (nM) |
|---|---|
| Example 1 | 15 |
| Example 2 | 197 |
| Example 3 | 9 |
| Example 4 | 69 |
| Example 5 | 164 |
| Example 6 | 62 |
| Example 7 | 8 |
| Example 8 | 3 |
| Example 9 | 480 |
| Example 10 | 10 |
| Example 11 | 265 |
| Example 12 | 90 |
| Example 13 | 341 |
| Example 14 | 291 |
| Example 15 | 153 |
| Example 16 | 84 |
| Example 17 | 474 |
| Example 18 | 40 |
| Example 19 | 18 |
| Example 20 | 340 |
| Example 21 | 209 |
| Example 22 | 27 |
| Example 23 | 207 |
| Example 24 | 93 |
| Example 25 | 76 |
| Example 26 | 43 |
| Example 27 | 292 |
| Example 28 | 710 |
| Example 29 | 10 |
| Example 30 | 130 |
| Example 31 | 42 |
| Example 32 | 77 |
| Example 33 | 170 |
| Example 34 | 453 |
| Example 35 | 8 |
| Example 36 | 487 |
| Example 37 | 12 |
| Example 38 | 890 |
| Example 39 | 3 |
| Example 40 | 266 |
| Example 41 | 59 |
| Example 42 | 458 |
| Example 43 | 177 |
| Example 44 | 770 |
| Example 45 | 552 |
| Example 46 | 92 |
| Example 47 | 155 |
| Example 48 | 237 |
| Example 49 | 22 |
| Example 50 | 537 |
| Example 51 | 118 |
| Example 52 | 128 |
| Example 53 | 357 |
| Example 54 | 322 |
| Example 55 | 276 |
| Example 56 | 234 |
| Example 57 | 974 |
| Example 58 | 92 |
| Example 59 | 11 |
| Example 60 | 4 |
| Example 61 | 68 |
| Example 62 | 7 |
| Example 63 | 18 |
| Example 64 | 93 |
| Example 65 | 68 |
| Example 66 | 221 |
| Example 67 | 14 |
| Example 68 | 17 |
| Example 69 | 9 |
| Example 70 | 5 |
| Example 71 | 11 |
| Example 72 | 3 |
| Example 73 | 9 |
| Example 74 | 14 |
| Example 75 | 17 |
| Example 76 | 4 |
| Example 77 | 12 |
| Example 78 | 34 |
| Example 79 | 110 |
| Example 80 | 2 |
| Example 81 | 6 |
| Example 82 | 282 |
| Example 83 | 361 |
| Example 84 | 52 |
| Example 85 | 15 |
| Example 86 | 42 |
| Example 87 | 18 |
| Example 88 | 13 |
| Example 89 | 37 |
| Example 90 | 27 |
| Example 91 | 27 |
| Example 92 | 3 |
| Example 93 | 8 |
| Example 94 | 68 |
| Example 95 | 189 |
| Example 96 | 16 |
| Example 97 | 486 |
| Example 98 | 272 |
| Example 99 | 21 |
| Example 100 | 138 |
| Example 101 | 19 |
| Example 102 | 296 |
| Example 103 | 542 |
| Example 104 | 176 |
| Example 105 | 909 |
| Example 106 | 695 |
| Example 107 | 58 |
| Example 108 | 651 |
| Example 109 | 29 |
| Example 110 | 312 |
| Example 111 | 727 |
| Example 112 | 82 |
| Example 113 | 838 |
| Example 114 | 37 |
| Example 115 | 36 |
| Example 116 | 74 |
| Example 117 | 692 |
| Example 118 | 31 |
| Example 119 | 482 |
| Example 120 | 123 |
| Example 121 | 24 |
| Example 122 | 571 |
| Example 123 | 223 |
| Example 124 | 23 |
| Example 125 | 453 |
| Example 126 | 843 |
| Example 127 | 73 |
| Example 128 | 27 |
| Example 129 | 124 |
| Example 130 | 5 |
| Example 131 | 54 |
| Example 132 | 7 |
| Example 133 | 92 |
| Example 134 | 166 |
| Example 135 | 100 |
| Example 136 | 29 |
| Example 137 | 574 |
| Example 138 | 25 |
| Example 139 | 123 |
| Example 140 | 10 |
| Example 141 | 5 |
| Example 142 | 33 |
| Example 143 | 179 |
| Example 144 | 383 |
| Example 145 | 5 |
| Example 146 | 244 |
| Example 147 | 325 |
| Example 148 | 164 |
| Example 149 | 132 |
| Example 150 | 4 |
| Example 151 | 25 |
| Example 152 | 167 |
| Example 153 | 593 |
| Example 154 | 49 |
| Example 155 | 13 |
| Example 156 | 8 |
| Example 157 | 11 |
| Example 158 | 335 |
| Example 159 | 170 |
| Example 160 | 36 |
| Example 161 | 22 |
| Example 162 | 120 |
| Example 163 | 104 |
| Example 164 | 17 |
| Example 165 | 7 |
| Example 166 | 9 |
| Example 167 | 7 |
| Example 168 | 7 |
| Example 169 | 68 |
| Example 170 | 34 |
| Example 171 | 10 |
| Example 172 | 49 |
| Example 173 | 334 |
| Example 174 | 16 |
| Example 175 | 80 |
| Example 176 | 7 |
| Example 177 | 114 |
| Example 178 | 267 |
| Example 179 | 238 |
| Example 180 | 75 |
| Example 181 | 239 |
| Example 182 | 610 |
| Example 183 | 139 |
| Example 184 | 77 |
| Example 185 | 25 |
| Example 186 | 274 |
| Example 187 | 196 |
| Example 188 | 155 |
| Example 189 | 107 |
| Example 190 | 499 |
| Example 191 | 12 |
| Example 192 | 264 |
| Example 193 | 958 |
| Example 194 | 7 |
| Example 195 | 532 |
| Example 196 | 11 |
| Example 197 | 28 |
| Example 198 | 17 |
| Example 199 | 140 |
| Example 200 | 537 |
| Example 201 | 226 |
| Example 202 | 36 |
| Example 203 | 31 |
| Example 204 | 74 |
| Example 205 | 33 |
| Example 206 | 226 |
| Example 207 | 327 |
| Example 208 | 711 |
| Example 209 | 146 |
| Example 210 | 109 |
| Example 211 | 14 |
| Example 212 | 882 |
| Example 213 | 3 |
| Example 214 | 251 |
| Example 215 | 127 |
| Example 216 | 326 |
| Example 217 | 648 |
| Example 218 | 184 |
| Example 219 | 154 |
| Example 220 | 195 |
| Example 221 | 722 |
| Example 222 | 172 |
| Example 223 | 58 |
| Example 224 | 832 |
| Example 225 | 856 |
| Example 226 | 349 |
| Example 227 | 69 |
| Example 228 | 51 |
| Example 229 | 8 |
| Example 230 | 6 |
| Example 231 | 30 |
| Example 232 | 6 |
| Example 233 | 10 |
| Example 234 | 9 |
| Example 235 | 388 |
| Example 236 | 12 |
| Example 237 | 7 |
| Example 238 | 76 |
| Example 239 | 591 |
| Example 240 | 163 |
| Example 241 | 11 |
| Example 242 | 705 |
| Example 243 | 23 |
| Example 244 | 587 |
| Example 245 | 23 |
| Example 246 | 104 |
| Example 247 | 916 |
| Example 248 | 303 |
| Example 249 | 15 |
| Example 250 | 2 |
| Example 251 | 84 |
| Example 252 | 15 |
| Example 253 | 209 |
| Example 254 | 995 |
| Example 255 | 45 |
| Example 256 | 3 |
| Example 257 | 19 |
| Example 258 | 492 |
| Example 259 | 26 |
| Example 260 | 69 |
| Example 261 | 25 |
| Example 262 | 85 |
| Example 263 | 33 |
| Example 264 | 480 |
| Example 265 | 16 |
| Example 266 | 26 |
| Example 267 | 145 |
| Example 268 | 8 |
| Example 269 | 10 |
| Example 270 | 20 |
| Example 271 | 27 |
| Example 272 | 12 |
| Example 273 | 511 |
| Example 274 | 31 |
| Example 275 | 25 |
| Example 276 | 113 |
| Example 277 | 179 |
| Example 278 | 15 |
| Example 279 | 8 |
| Example 280 | 859 |
| Example 281 | 11 |
| Example 282 | 5 |
| Example 283 | 165 |
| Example 284 | 74 |
| Example 285 | 184 |
| Example 286 | 72 |
| Example 287 | 9 |
| Example 288 | 39 |
| Example 289 | 301 |
| Example 290 | 209 |
| Example 291 | 12 |
| Example 292 | 5 |
| Example 293 | 21 |
| Example 294 | 251 |
| Example 295 | 81 |
| Example 296 | 418 |
| Example 297 | 79 |
| Example 298 | 14 |
| Example 299 | 215 |
| Example 300 | 22 |
| Example 301 | 8 |
| Example 302 | 5 |
| Example 303 | 6 |
| Example 304 | 4 |
| Example 305 | 29 |
| Example 306 | 654 |
| Example 307 | 73 |
| Example 308 | 75 |
| Example 309 | 720 |
| Example 310 | 182 |
| Example 311 | 294 |
| Example 312 | 12 |
| Example 313 | 26 |
| Example 314 | 12 |
| Example 315 | 86 |
| Example 316 | 241 |
| Example 317 | 21 |
| Example 318 | 209 |
| Example 319 | 8 |
| Example 320 | 27 |
| Example 321 | 51 |
| Example 322 | 26 |
| Example 323 | 205 |
| Example 324 | 274 |
| Example 325 | 274 |
| Example 326 | 263 |
| Example 327 | 179 |
| Example 328 | 306 |
| Example 329 | 149 |
| Example 330 | 69 |
| Example 331 | 382 |
| Example 332 | 8 |
| Example 333 | 6 |
| Example 334 | 446 |
| Example 335 | 10 |
| Example 336 | 9 |
| Example 337 | 53 |
| Example 338 | 24 |
| Example 339 | 174 |
| Example 340 | 36 |
| Example 341 | 271 |
| Example 342 | 5 |
| Example 343 | 92 |
| Example 344 | 67 |
| Example 345 | 175 |
| Example 346 | 108 |
| Example 347 | 7 |
| Example 348 | 880 |
| Example 349 | 14 |

What is claimed is:

1. A compound of Formula I: 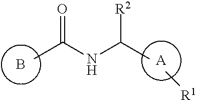
or a pharmaceutically acceptable salt thereof, wherein:
A is a five or six membered, nitrogen-containing heteroaryl ring;
B is a five-membered heteroaryl, bicyclic heterocycloalkyl or bicyclic heteroaryl, wherein the bicyclic heterocycloalkyl and bicyclic heteroaryl are unsubstituted or substituted with one to three substituents and the five-membered heteroaryl is substituted with one to three substituents, wherein the substituents are independently selected from the group consisting of aryl, haloC₁-C₆alkyl, C₁-C₆alkyl, —C₁-C₆alkylOH, alkoxy, —OH, C₁-C₆alkylaryl, C₃-C₆cycloalkyl, C₁-C₆alkylC₃-C₆cycloalkyl, heteroaryl, —C₁-C₆alkylCON(R^{a})₂, haloC₁-C₆alkoxy, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl, halogen, and —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, and —C₁-C₆alkylCOOH is unsubstituted or substituted with one to three substituents independently selected from the group consisting of C₁-C₆alkyl, halogen, —NH₂, —OH, —CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl;
R¹ is C₃-C₆cycloalkyl, cycloheteroalkyl, aryl or heteroaryl, wherein the C₃-C₆cycloalkyl, cycloheteroalkyl, aryl or heteroaryl is unsubstituted or substituted with one to three substituents independently selected from the group consisting of alkoxy, —CN, —C₁-C₆alkylOH, halogen, C₁-C₆alkyl, haloC₁-C₆alkyl, and —OH;
each occurrence of R^{a} is independently hydrogen, C₁-C₆alkyl or haloC₁-C₆alkyl; and
R² is hydrogen, C₁-C₆alkyl or haloC₁-C₆alkyl.

2. The compound of claim 1, or a pharmaceutically salt thereof, wherein A is a five-membered, nitrogen-containing heteroaryl ring.

3. The compound of claim 1, or a pharmaceutically salt thereof, wherein A is a five-membered nitrogen-containing heteroaryl ring, wherein the five-membered nitrogen-containing heteroaryl ring is selected from the group consisting of pyrrolidine, pyrroline, pyrazolidine, pyrazoline, imidazolidine, imidazoline, pyrrole, pyrazole, imidazole, 1H-1,2,3-triazole, 4H-1,2,4-triazole, isoaxazole, oxazole, 1,2,3-oxadiazole, 1,3,4-oxadiazole, furazan, 1,2,4-oxadiazole, 1,2,3,4-oxatrizole, 1,2,3,5-oxatriazole, isothiazole, thiazole, 1,2,3-thiadiazole, 1,3,4-thiadiazole, 1,2,5-thiadiazole, 1,2,4-thiadiazole, 1,2,3,4-thiatrizole and 1,2,3,5-thiatriazole.

4. The compound of claim 1, or a pharmaceutically salt thereof, wherein A is

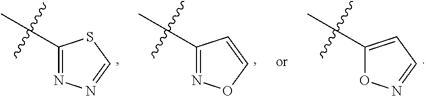 [SMILES: *c1nncs1 ; *c1ccon1 ; *c1ccno1]

5. The compound of claim 1, or a pharmaceutically salt thereof, wherein A is a six-membered, nitrogen-containing heteroaryl ring.

6. The compound of claim 5, or a pharmaceutically salt thereof, wherein the six-membered, nitrogen-containing heteroaryl ring is selected from the group consisting of pyridine, pyrazine, pyrimidine, and pyridazine.

7. The compound of claim 5, or a pharmaceutically salt thereof, wherein A is

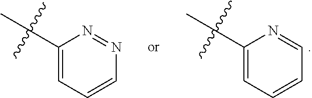 [SMILES: *c1cccnn1 ; *c1ccccn1]

8. The compound of claim 1, or a pharmaceutically salt thereof, wherein R¹ is aryl, wherein the aryl is phenyl.

9. The compound of claim 8, or a pharmaceutically salt thereof, wherein R¹ is unsubstituted.

10. The compound of claim 8, or a pharmaceutically salt thereof, wherein R¹ is substituted with one to three substituents independently selected from the group consisting of —OH, methyl, chlorine and fluorine.

11. The compound of claim 1, or a pharmaceutically salt thereof, wherein R¹ is C₃-C₆cycloalkyl, wherein the C₃-C₆cycloalkyl is cyclopentane or cyclohexane.

12. The compound of claim 1, or a pharmaceutically salt thereof, wherein R¹ is C₃-C₆cycloalkyl, wherein the C₃-C₆cycloalkyl is unsubstituted.

13. The compound of claim 1, or a pharmaceutically salt thereof, wherein R¹ is C₃-C₆cycloalkyl, wherein the C₃-C₆cycloalkyl is substituted with one to three substituents independently selected from the group consisting of —OH, methyl, chlorine and fluorine.

14. The compound of claim 1, or a pharmaceutically salt thereof, wherein R¹ is heterocycloalkyl, wherein the heterocycloalkyl is piperidine.

15. The compound of claim 1, or a pharmaceutically salt thereof, wherein R¹ is heterocycloalkyl, wherein the heterocycloalkyl is unsubstituted.

16. The compound of claim 1, or a pharmaceutically salt thereof, wherein R¹ is heterocycloalkyl, wherein the heterocycloalkyl is substituted with one to three methyl groups.

17. The compound claim 1, or a pharmaceutically salt thereof, wherein R¹ is heteroaryl, wherein the heteroaryl is pyridine, thiophene, thiazole, triazole or pyrazole.

18. The compound claim 1, or a pharmaceutically salt thereof, wherein R¹ is heteroaryl, wherein the heteroaryl is unsubstituted.

19. The compound claim 1, or a pharmaceutically salt thereof, wherein R¹ is heteroaryl, wherein the heteroaryl is substituted with one to three methyl groups.

20. The compound claim 1, or a pharmaceutically salt thereof, wherein B is 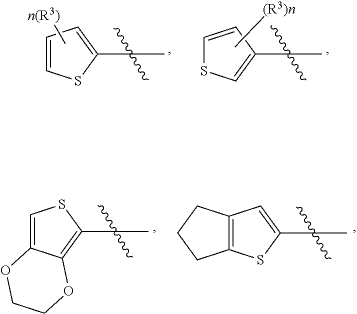
wherein each occurrence of R³ is independently selected from the group consisting of —OC₁-C₆alkyl, C₁-C₆alkyl and halogen; and
n is 1 or 2.

21. The compound of claim 20, or a pharmaceutically salt thereof, wherein each occurrence of R³ is independently selected from the group consisting of ethoxy, methoxy, methyl and chlorine.

22. The compound of claim 1, or a pharmaceutically salt thereof, wherein B is 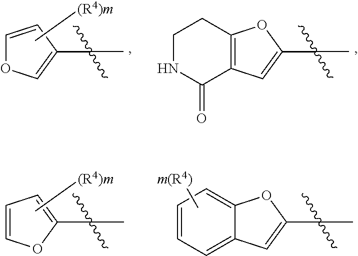
wherein each occurrence of R⁴ is independently selected from the group consisting of —OH, —C₁-C₆alkylOC₁-C₆alkyl, —C₁-C₆alkyl and -haloC₁-C₆alkyl; and
m is 1, 2 or 3.

23. The compound of claim 22, or a pharmaceutically salt thereof, wherein each occurrence of R⁴ is independently selected from the group consisting of —OH, trifluoromethyl, methyl and —CH₂OCH₃.

24. The compound of claim 1, or a pharmaceutically salt thereof, wherein B is 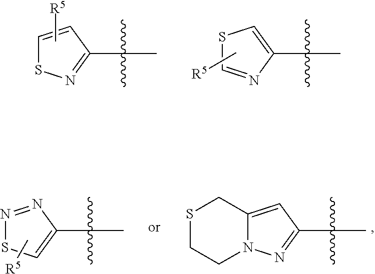
wherein R⁵ is methyl or CH₂phenyl.

25. The compound of claim 1, or a pharmaceutically salt thereof, wherein B is 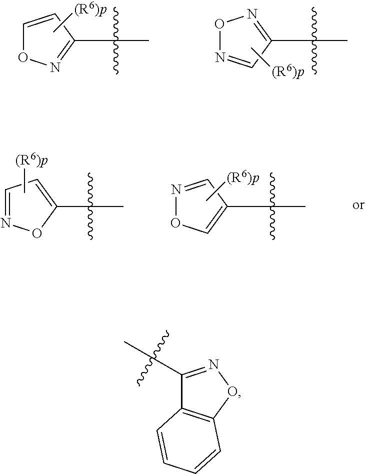
wherein each occurrence of R⁶ is independently selected from the group consisting of haloC₁-C₆alkyl, C₁-C₆alkyl, —C₁-C₆alkylOH, alkoxy, heteroaryl and C₃-C₆cycloalkyl, wherein the heteroaryl is unsubstituted or substituted with one to three C₁-C₆alkyls; and
p is 1, 2, or 3.

26. The compound of claim 25, or a pharmaceutically salt thereof, wherein each occurrence of R⁶ is independently selected from the group consisting of methyl, butyl, cyclopropyl, propyl, ethanol, butanol, dimethylpyridine, methoxy and trifluorophenyl.

27. The compound of claim 1, or a pharmaceutically salt thereof, wherein B is 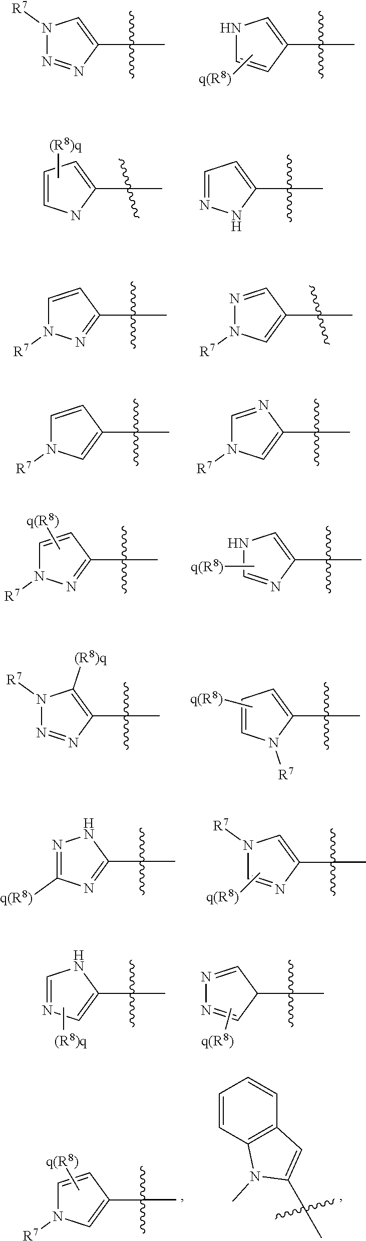 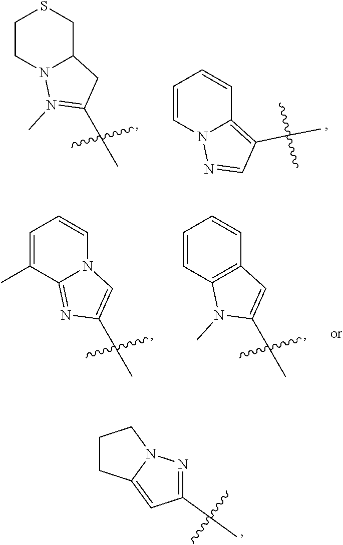
wherein R⁷ is aryl, haloC₁-C₆alkyl, C₁-C₆alkyl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl or —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, or —C₁-C₆alkylCOOH is unsubstituted or substituted with one to three substituents independently selected from the group consisting of C₁-C₆alkyl, halogen, —NH₂, —OH, CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl;
each occurrence of R⁸ is independently selected from the group consisting of C₁-C₆alkyl, —C₁-C₆alkylOH, C₃-C₆cycloalkyl, and halogen;
R² is hydrogen, C₁-C₆alkyl or haloC₁-C₆alkyl;
R^{a} is hydrogen, C₁-C₆alkyl or haloC₁-C₆alkyl; and
q is 1, 2, or 3.

28. The compound of claim 27, or a pharmaceutically salt thereof, wherein R⁷ is dimethylphenyl, dimethylpyridyl, fluoromethyl, fluoroethyl, fluorobutyl, difluoroethyl, difluorobutyl, trifluoromethyl, trifluoroethyl, trifluorobutyl, fluoropentyl, methyl, butyl, hexyl, cyclopropyl, cyclobutyl, cyclohexane, propyl, ethanol, propanol, butanol, dimethylpyridine, methoxy, —CH₂CONH₂ and pyrazole, tetrahydrofuran, trifluorophenyl, oxetane, cyanomethyl, methylphenyl, difluorocyclobutyl, —CH₂SO₂CH₃, tetrahydropyran, pyridine, pyrimidine, pyrazine,

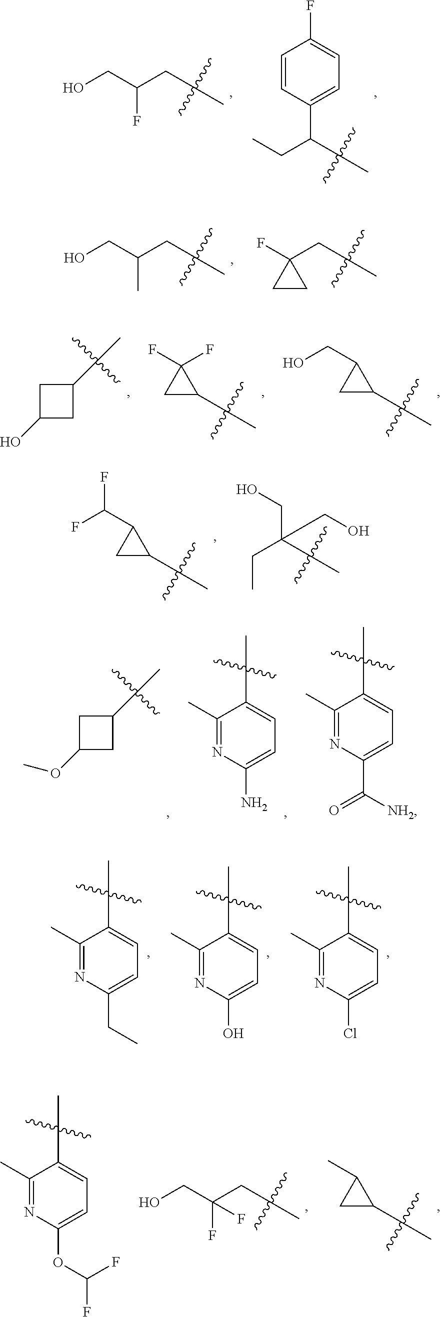

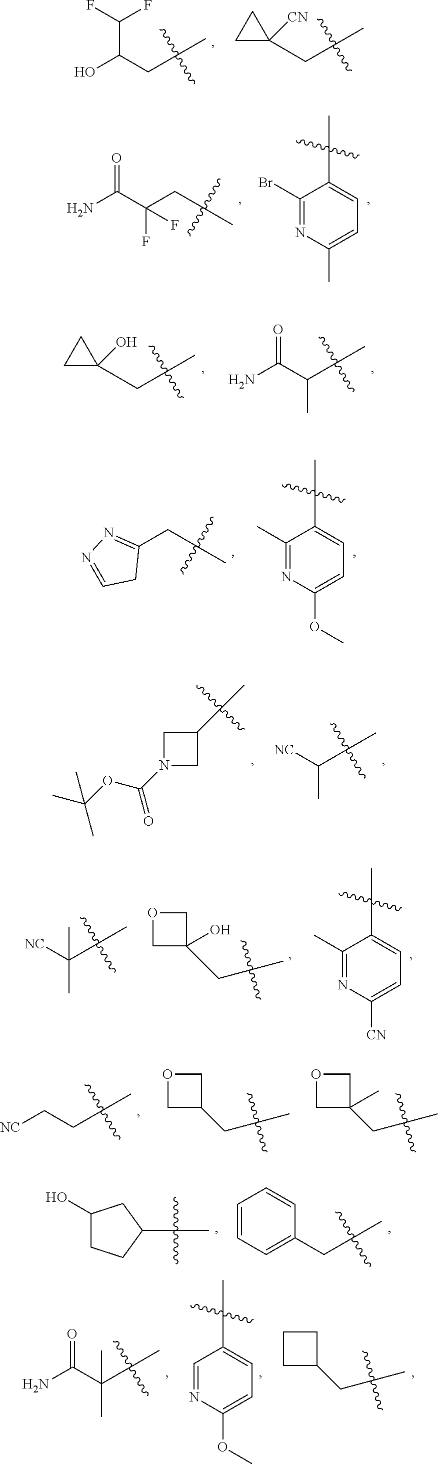

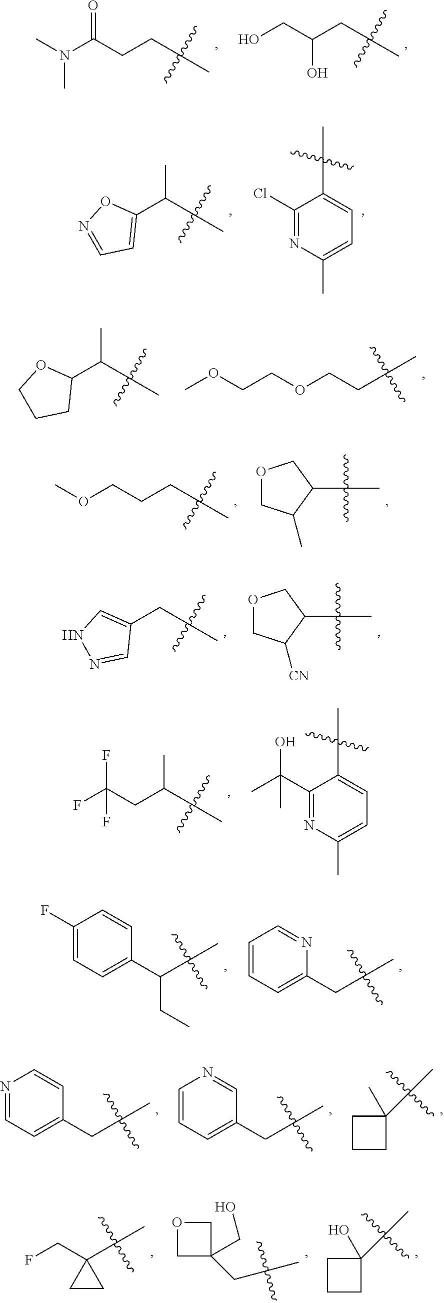

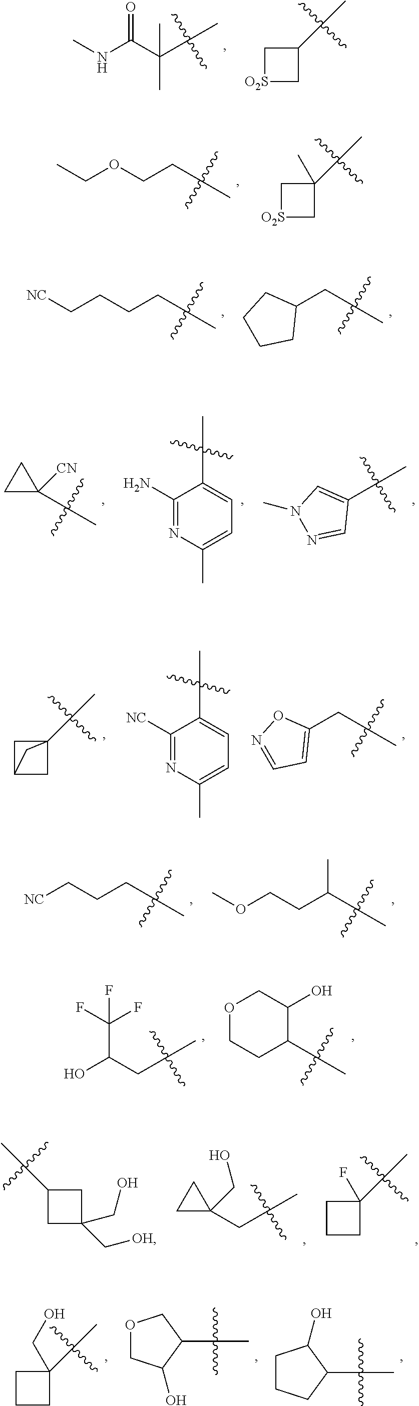

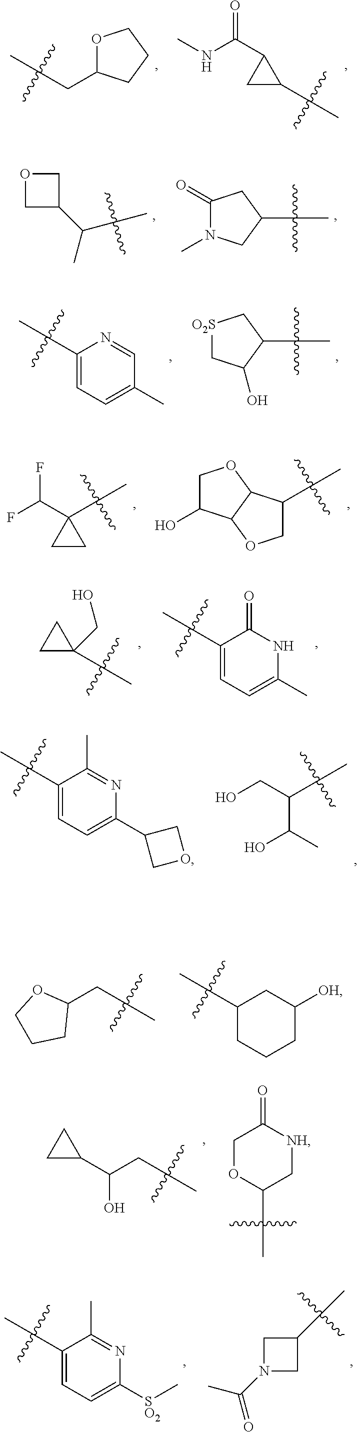

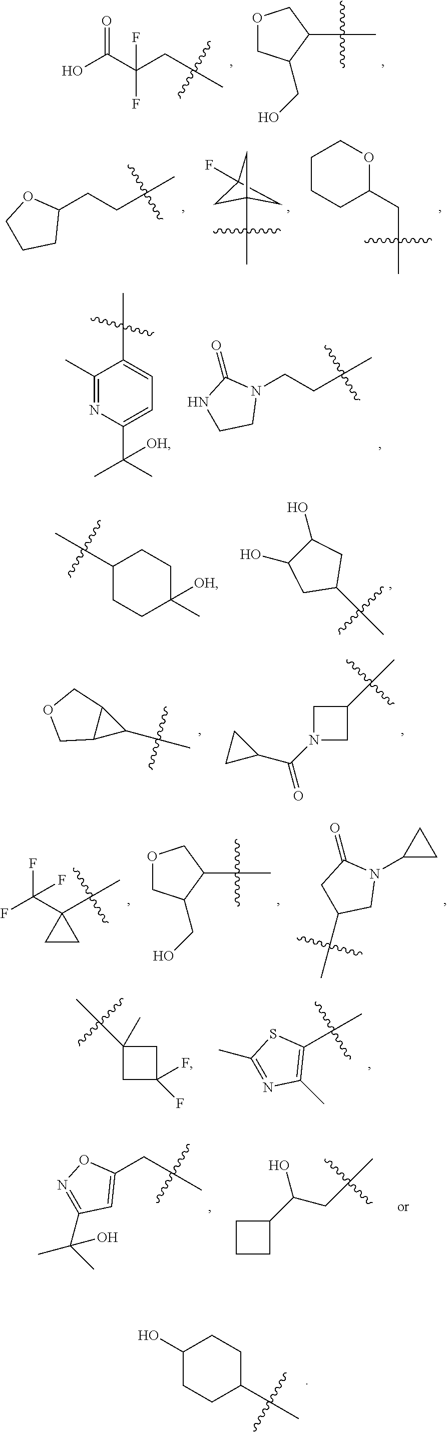

29. The compound of claim 1, or a pharmaceutically salt thereof, wherein B is 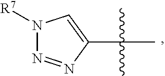
wherein R⁷ is dimethylphenyl, fluoromethyl, fluoroethyl, fluorobutyl, difluoroethyl, difluorobutyl, trifluoromethyl, trifluoroethyl, trifluorobutyl, fluoropentyl, methyl, butyl, hexyl, cyclopropyl, cyclobutyl, cyclohexane, propyl, ethanol, propanol, butanol, dimethylpyridine, methoxy, —CH₂CONH₂ and pyrazole, tetrahydrofuran, trifluorophenyl, oxetane, cyanomethyl, methylphenyl, difluorocyclobutyl, —CH₂SO₂CH₃, tetrahydropyran, pyridine, pyrimidine, pyrazine,

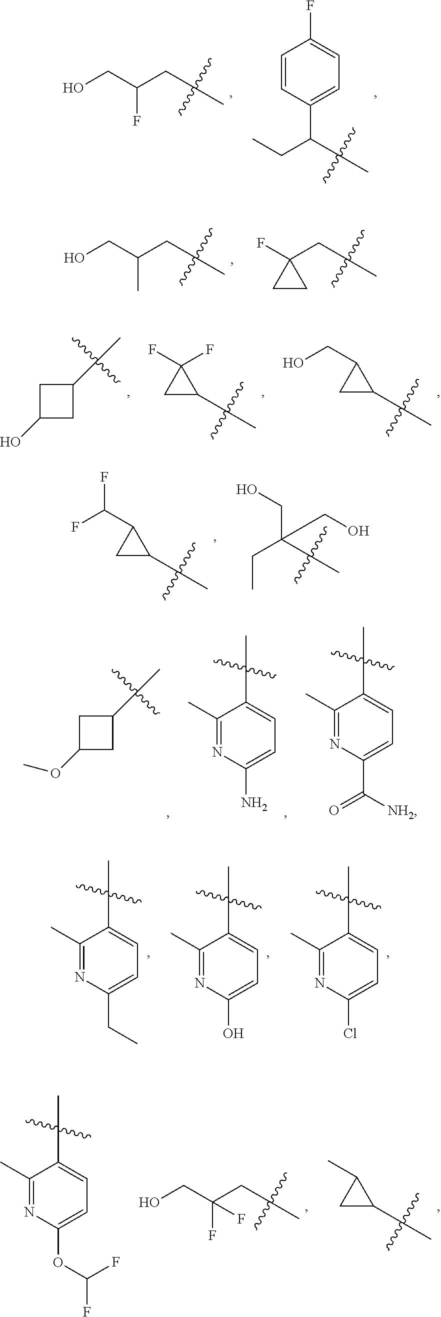

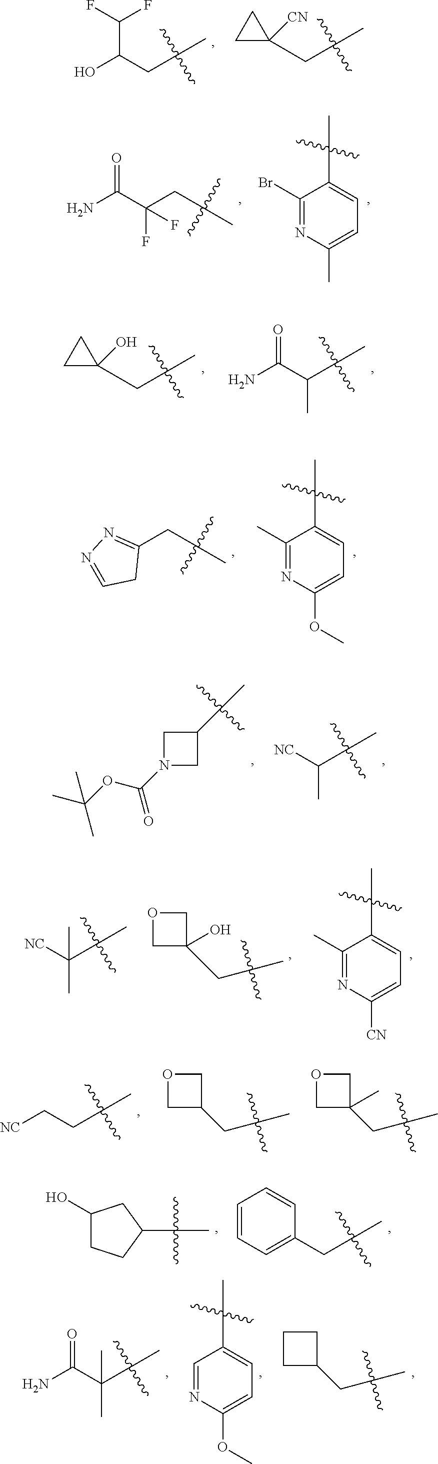

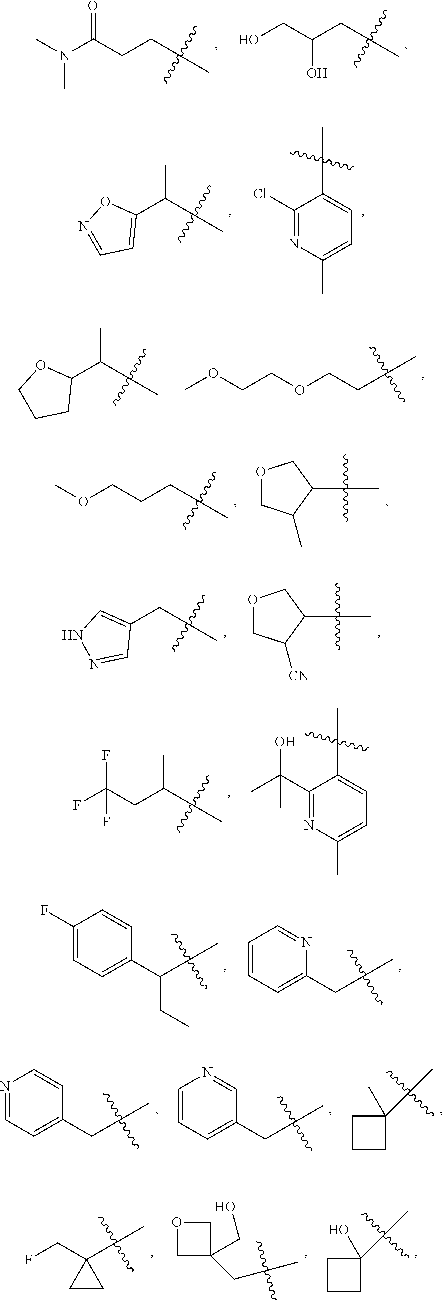

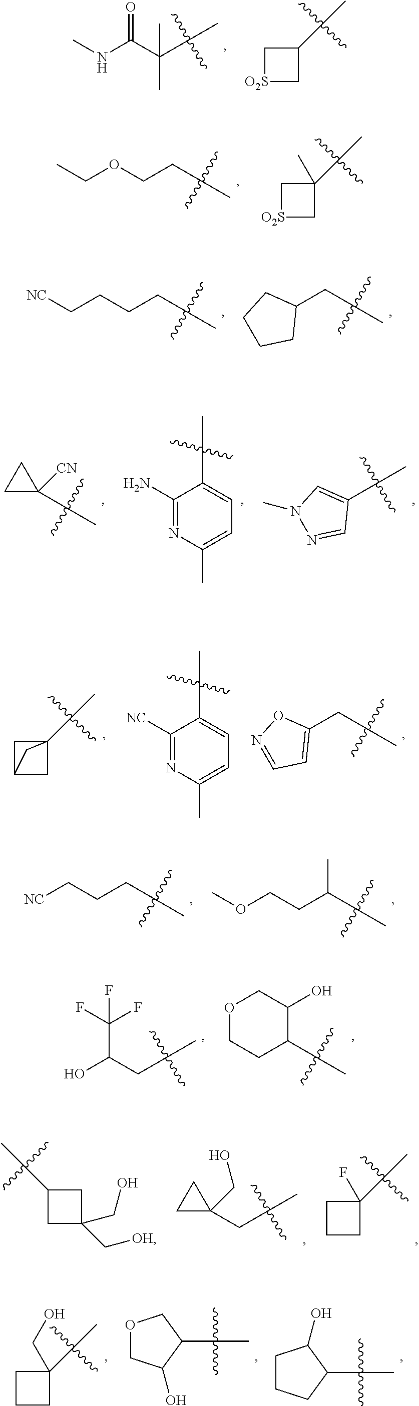

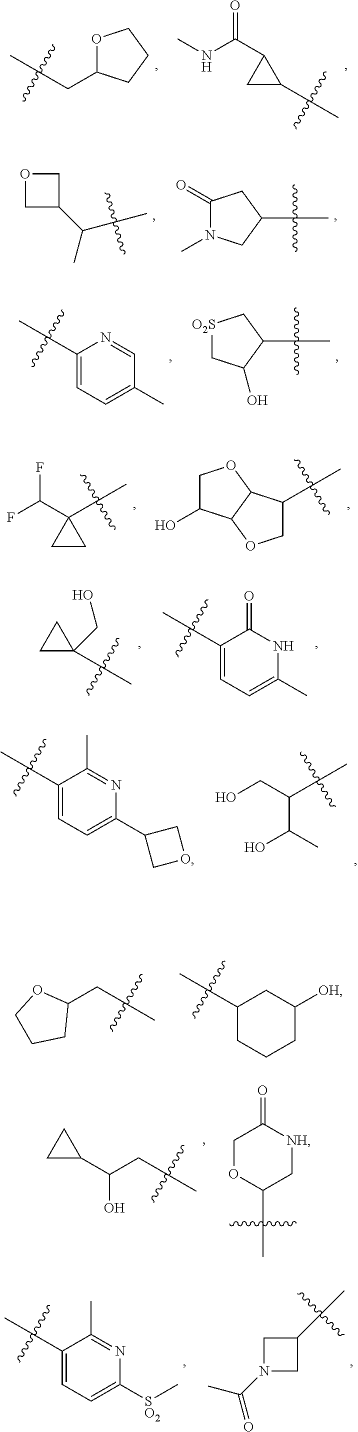

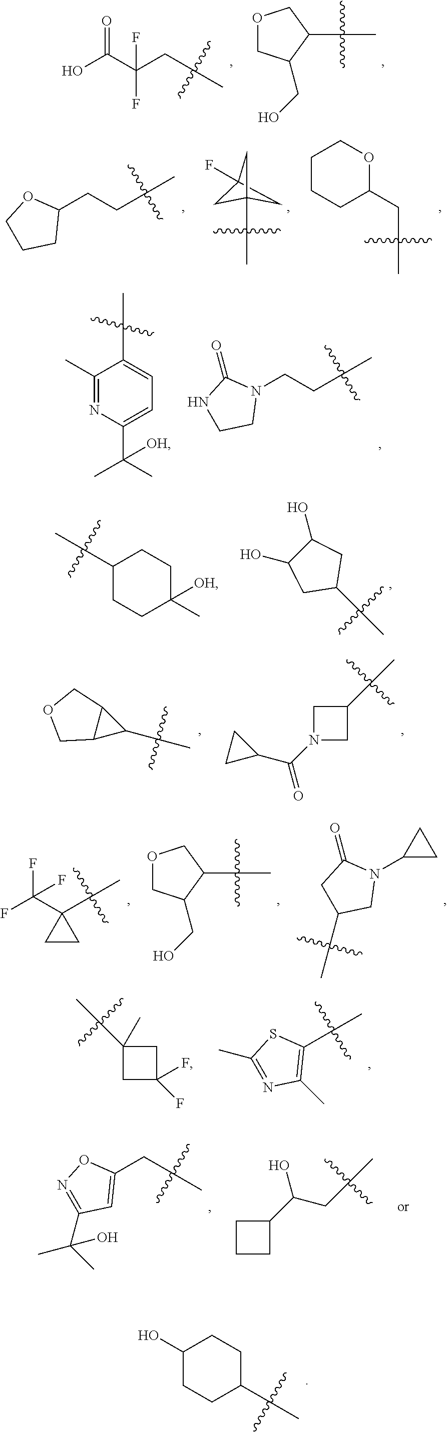

30. The compound of claim 1, or a pharmaceutically salt thereof, wherein R⁷ is dimethylpyridine.

31. A compound of Formula IA: 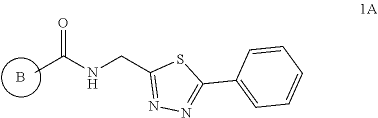
or a pharmaceutically acceptable salt thereof, wherein:
B is a five-membered heteroaryl, wherein the five-membered heteroaryl is substituted with one to three substituents, wherein the substituents are independently selected from the group consisting of aryl, haloC₁-C₆alkyl, C₁-C₆alkyl, —C₁-C₆alkylOH, alkoxy, —OH, C₁-C₆alkylaryl, C₃-C₆cycloalkyl, C₁-C₆alkylC₃-C₆cycloalkyl, heteroaryl, —C₁-C₆alkylCON(R^{a})₂, haloC₁-C₆alkoxy, C₁-C₆alkylheteroaryl, heterocycloalkyl, —C₁-C₆alkylCN, C₁-C₆alkylheterocycloalkyl, —C₁-C₆alkylOC₁-C₆alkylOC₁-C₆alkyl, —C₁-C₆alkylOC₁-C₆alkyl, —C₁-C₆alkylCO₂C₁-C₆alkyl, —C₁-C₆alkylSO₂C₁-C₆alkyl, halogen, and —C₁-C₆alkylCOOH, wherein the aryl, —C₁-C₆alkylOH, C₁-C₆alkylaryl, C₃-C₆cycloalkyl, C₁-C₆alkylC₃-C₆cycloalkyl, heteroaryl, —C₁-C₆alkylCON(R^{a})₂, heterocycloalkyl, C₁-C₆alkylheterocycloalkyl, and —C₁-C₆alkylCOOH is unsubstituted or substituted with one to three substituents independently selected from the group consisting of C₁-C₆alkyl, halogen, —NH₂, —OH, —CON(R^{a})₂, alkoxy, —CO₂C₁-C₆alkyl, —CN, —C₁-C₆alkylOH, haloC₁-C₆alkyl, oxetane, —SO₂C₁-C₆alkyl, —COC₁-C₆alkyl, —COC₃-C₆cycloalkyl and C₃-C₆cycloalkyl; and
R^{a} is hydrogen, C₁-C₆alkyl or haloC₁-C₆alkyl.

32. The compound of claim 31, or a pharmaceutically salt thereof, wherein B is 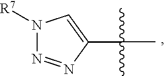
wherein R⁷ is dimethylphenyl, fluoromethyl, fluoroethyl, fluorobutyl, difluoroethyl, difluorobutyl, trifluoromethyl, trifluoroethyl, trifluorobutyl, fluoropentyl, methyl, butyl, hexyl, cyclopropyl, cyclobutyl, cyclohexane, propyl, ethanol, propanol, butanol, dimethylpyridine, methoxy, —CH₂CONH₂ and pyrazole, tetrahydrofuran, trifluorophenyl, oxetane, cyanomethyl, methylphenyl, difluorocyclobutyl, —CH₂SO₂CH₃, tetrahydropyran, pyridine, pyrimidine, pyrazine,

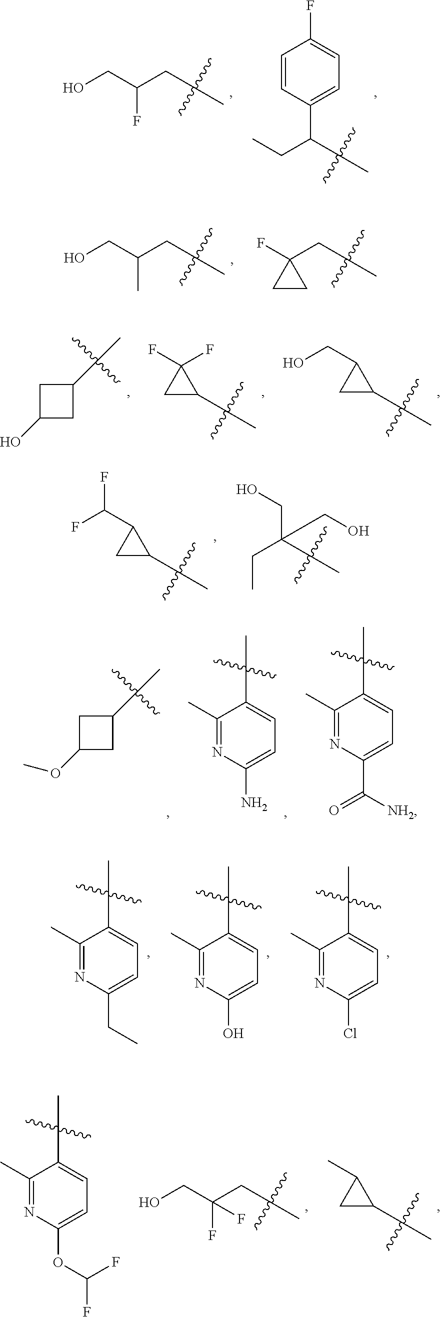

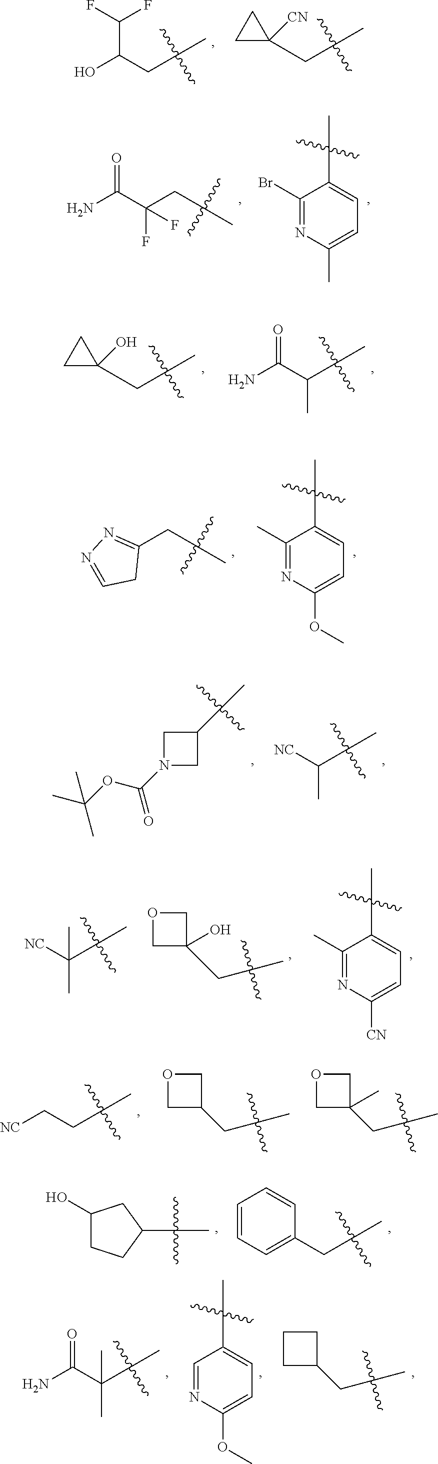

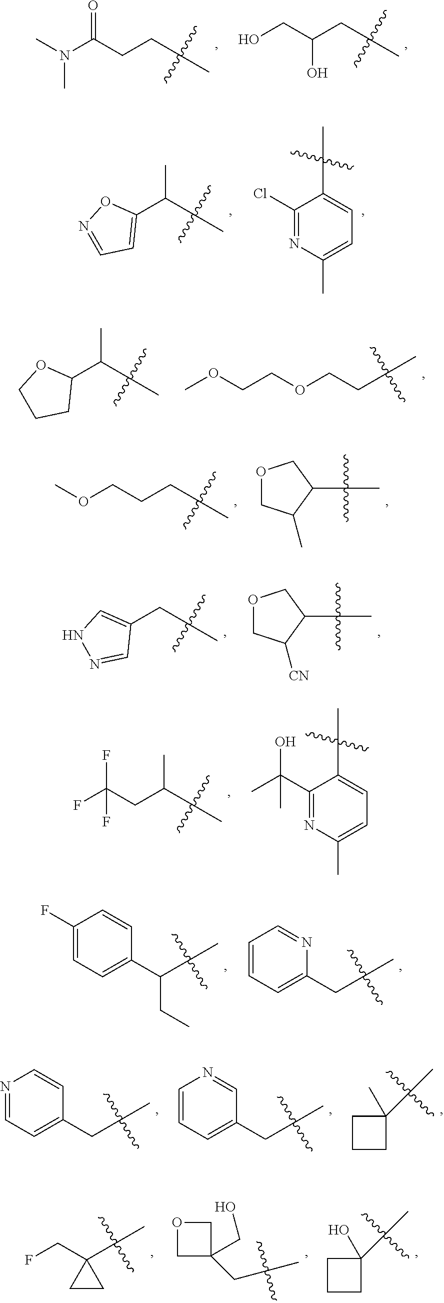

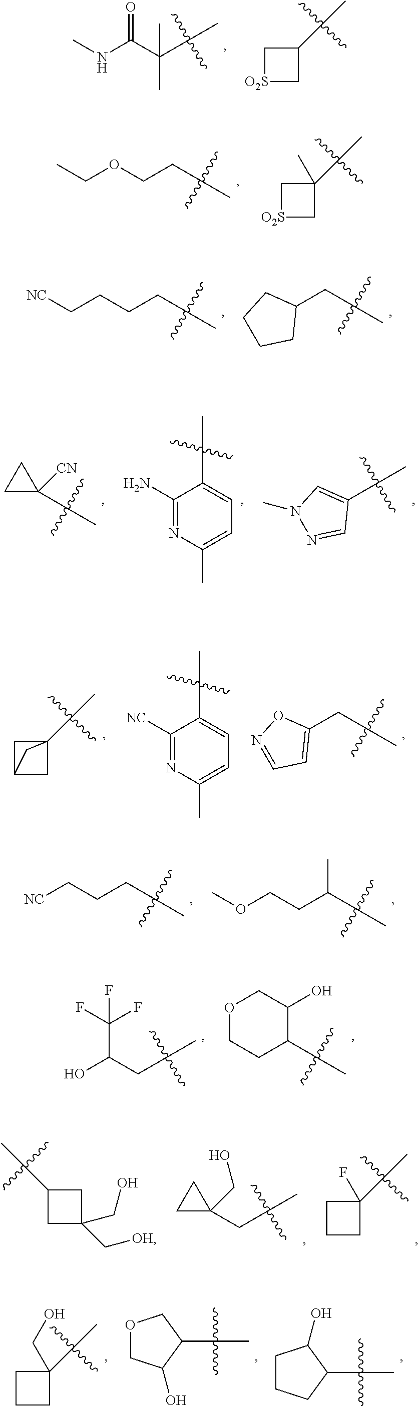

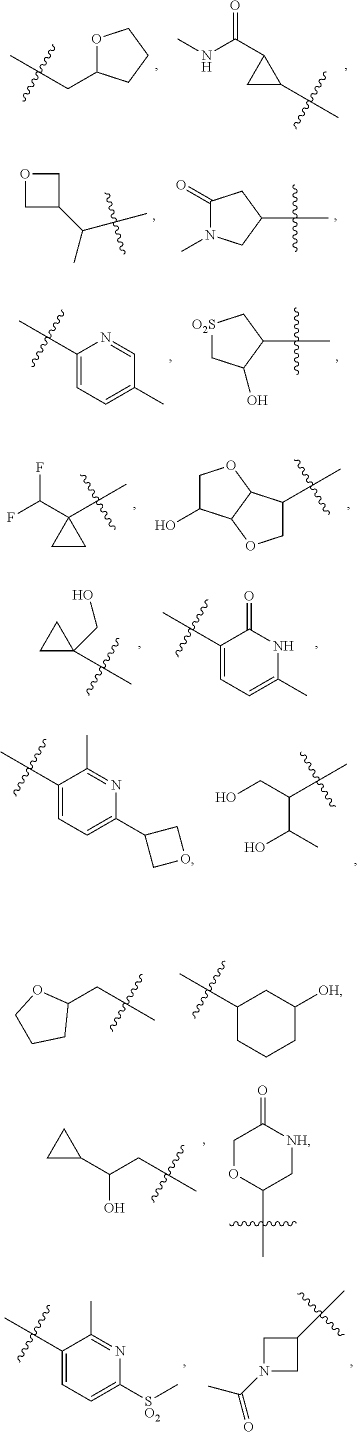

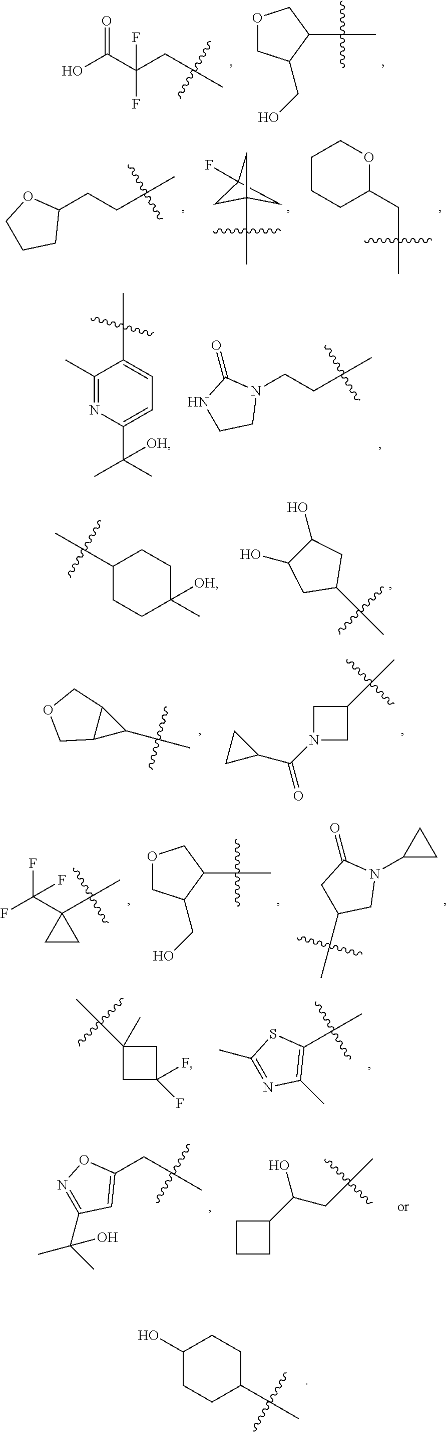

33. A compound selected from the group consisting of:

| Structure | Name |
|---|---|
| 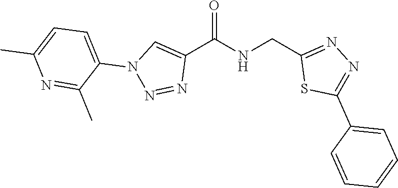 | 1-(2,6-dimethylpyridin-3-yl)-N-((5-phenyl-1,3,4- thiadiazol-2-yl)methyl)-1H-1,2,3-triazole-4- carboxamide |
| 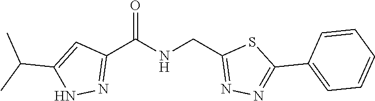 [SMILES: CC(C)c1cc(C(=O)NCc2nnc(-c3ccccc3)s2)n[nH]1] | 5-isopropyl-N-[(5-phenyl-1,3,4-thiadiazol-2- yl)methyl]-1H-pyrazole-3-carboxamide |
| 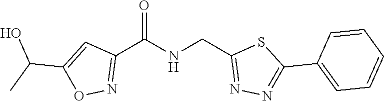 [SMILES: CC(O)c1cc(C(=O)NCc2nnc(-c3ccccc3)s2)no1] | 5-(1-hydroxyethyl)-N-[(5-phenyl-1,3,4-thiadiazol- 2-yl)methyl]isoxazole-3-carboxamide |
| 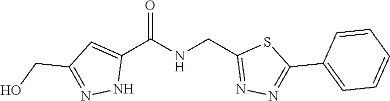 [SMILES: O=C(NCc1nnc(-c2ccccc2)s1)c1cc(CO)n[nH]1] | 3-(hydroxymethyl)-N-((5-phenyl-1,3,4-thiadiazol- 2-yl)methyl)-1H-pyrazole-5-carboxamide |
| 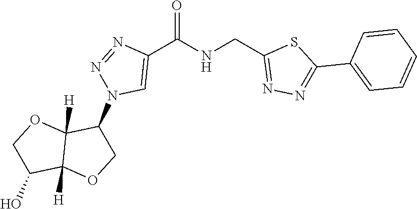 [SMILES: O=C(NCc1nnc(-c2ccccc2)s1)c1cn([C@H]2CO[C@H]3[C@@H]2OC[C@H]3O)nn1] | 1-[(3S,3aR,6R,6aR)-6-hydroxy-2,3,3a,5,6,6a- hexahydrofuro[3,2-b]furan-3-yl]-N-[(5-phenyl- 1,3,4-thiadiazol-2-yl)methyl]triazole-4- carboxamide |
| 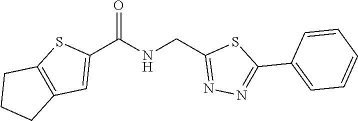 [SMILES: O=C(NCc1nnc(-c2ccccc2)s1)c1cc2c(s1)CCC2] | N-[(5-phenyl-1,3,4-thiadiazol-2-yl)methyl]-5,6- dihydro-4H-cyclopenta[b]thiophene-2-carboxamide |
| 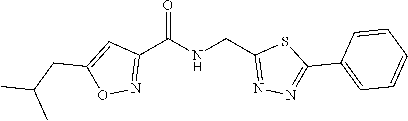 [SMILES: CC(C)Cc1cc(C(=O)NCc2nnc(-c3ccccc3)s2)no1] | 5-isobutyl-N-[(5-phenyl-1,3,4-thiadiazol-2- yl)methyl]isoxazole-3-carboxamide |
| 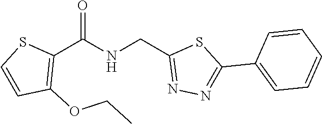 [SMILES: CCOc1ccsc1C(=O)NCc1nnc(-c2ccccc2)s1] | 3-ethoxy-N-[(5-phenyl-1,3,4-thiadiazol-2- yl)methyl]thiophene-2-carboxamide |
| 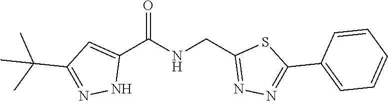 [SMILES: CC(C)(C)c1cc(C(=O)NCc2nnc(-c3ccccc3)s2)[nH]n1] | 3-(tert-butyl)-N-((5-phenyl-1,3,4-thiadiazol-2- yl)methyl)-1H-pyrazole-5-carboxamide |
| 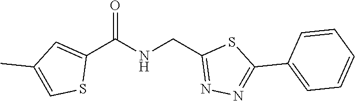 [SMILES: Cc1csc(C(=O)NCc2nnc(-c3ccccc3)s2)c1] | 4-methyl-N-[(5-phenyl-1,3,4-thiadiazol-2- yl)methyl]thiophene-2-carboxamide |
| 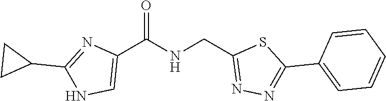 [SMILES: O=C(NCc1nnc(-c2ccccc2)s1)c1c[nH]c(C2CC2)n1] | 2-cyclopropyl-N-((5-phenyl-1,3,4-thiadiazol-2- yl)methyl)-1H-imidazole-4-carboxamide |
| 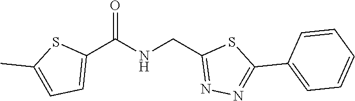 [SMILES: Cc1ccc(C(=O)NCc2nnc(-c3ccccc3)s2)s1] | 5-methyl-N-[(5-phenyl-1,3,4-thiadiazol-2- yl)methyl]thiophene-2-carboxamide |
| 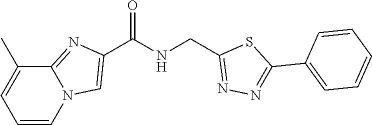 [SMILES: Cc1cccn2cc(C(=O)NCc3nnc(-c4ccccc4)s3)nc12] | 8-methyl-N-((5-phenyl-1,3,4-thiadiazol-2- yl)methyl)imidazo[1,2-a]pyridine-2-carboxamide |
| 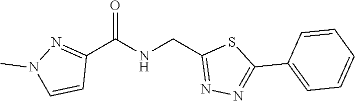 [SMILES: Cn1ccc(C(=O)NCc2nnc(-c3ccccc3)s2)n1] | 1-methyl-N-[(5-phenyl-1,3,4-thiadiazol-2- yl)methyl]pyrazole-3-carboxamide |
| 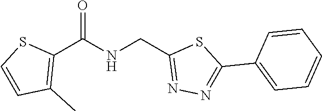 [SMILES: Cc1ccsc1C(=O)NCc1nnc(-c2ccccc2)s1] | 3-methyl-N-[(5-phenyl-1,3,4-thiadiazol-2- yl)methyl]thiophene-2-carboxamide |
| 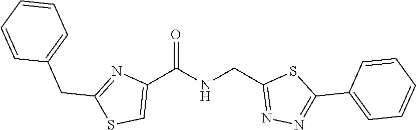 [SMILES: O=C(NCc1nnc(-c2ccccc2)s1)c1csc(Cc2ccccc2)n1] | 2-benzyl-N-((5-phenyl-1,3,4-thiadiazol-2- yl)methyl)thiazole-4-carboxamide |
| 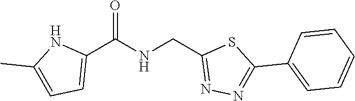 [SMILES: Cc1ccc(C(=O)NCc2nnc(-c3ccccc3)s2)[nH]1] | 5-methyl-N-[(5-phenyl-1,3,4-thiadiazol-2- yl)methyl]-1H-pyrrole-2-carboxamide |
| 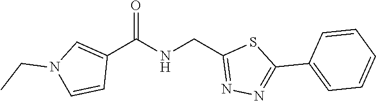 [SMILES: CCn1ccc(C(=O)NCc2nnc(-c3ccccc3)s2)c1] | 1-ethyl-N-[(5-phenyl-1,3,4-thiadiazol-2- yl)methyl]pyrrole-3-carboxamide |
| 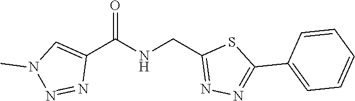 [SMILES: Cn1cc(C(=O)NCc2nnc(-c3ccccc3)s2)nn1] | 1-methyl-N-[(5-phenyl-1,3,4-thiadiazol-2- yl)methyl]triazole-4-carboxamide |
| 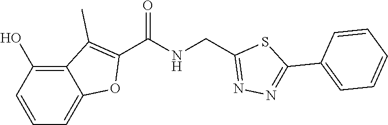 [SMILES: Cc1c(C(=O)NCc2nnc(-c3ccccc3)s2)oc2cccc(O)c12] | 4-hydroxy-3-methyl-N-[(5-phenyl-1,3,4-thiadiazol- 2-yl)methyl]benzofuran-2-carboxamide |
| 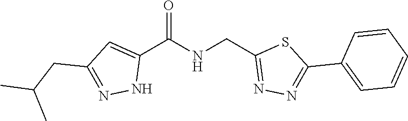 [SMILES: CC(C)Cc1cc(C(=O)NCc2nnc(-c3ccccc3)s2)[nH]n1] | 3-isobutyl-N-((5-phenyl-1,3,4-thiadiazol-2- yl)methyl)-1H-pyrazole-5-carboxamide |
| 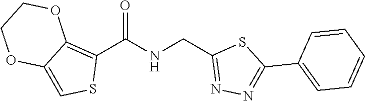 [SMILES: O=C(NCc1nnc(-c2ccccc2)s1)c1scc2c1OCCO2] | N-[(5-phenyl-1,3,4-thiadiazol-2-yl)methyl]-2,3- dihydrothieno[3,4-b][1,4]dioxine-5-carboxamide |
| 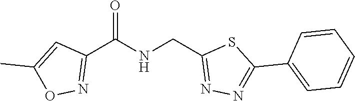 [SMILES: Cc1cc(C(=O)NCc2nnc(-c3ccccc3)s2)no1] | 5-methyl-N-[(5-phenyl-1,3,4-thiadiazol-2- yl)methyl]isoxazole-3-carboxamide |
| 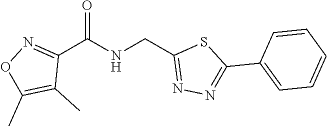 [SMILES: Cc1onc(C(=O)NCc2nnc(-c3ccccc3)s2)c1C] | 4,5-dimethyl-N-[(5-phenyl-1,3,4-thiadiazol-2- yl)methyl]isoxazole-3-carboxamide |
| 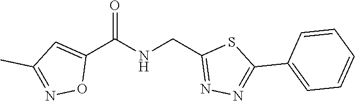 [SMILES: Cc1cc(C(=O)NCc2nnc(-c3ccccc3)s2)on1] | 3-methyl-N-[(5-phenyl-1,3,4-thiadiazol-2- yl)methyl]isoxazole-5-carboxamide |
| 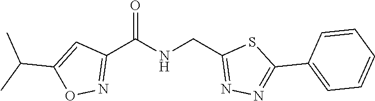 [SMILES: CC(C)c1cc(C(=O)NCc2nnc(-c3ccccc3)s2)no1] | 5-isopropyl-N-[(5-phenyl-1,3,4-thiadiazol-2- yl)methyl]isoxazole-3-carboxamide |
| 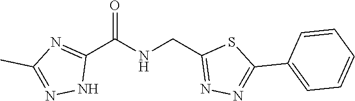 [SMILES: Cc1n[nH]c(C(=O)NCc2nnc(-c3ccccc3)s2)n1] | 3-methyl-N-((5-phenyl-1,3,4-thiadiazol-2- yl)methyl)-1H-1,2,4-triazole-5-carboxamide |
| 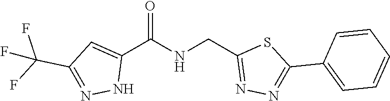 [SMILES: O=C(NCc1nnc(-c2ccccc2)s1)c1cc(C(F)(F)F)n[nH]1] | N-[(5-phenyl-1,3,4-thiadiazol-2-yl)methyl]-3- (trifluoromethyl)-1H-pyrazole-5-carboxamide |
| 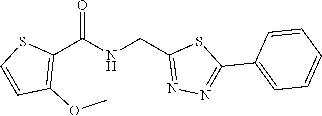 [SMILES: COc1ccsc1C(=O)NCc1nnc(-c2ccccc2)s1] | 3-methoxy-N-[(5-phenyl-1,3,4-thiadiazol-2- yl)methyl]thiophene-2-carboxamide |
| 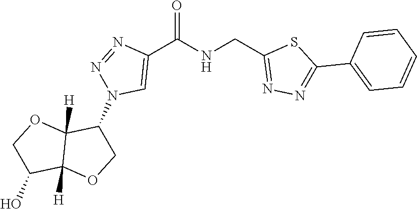 [SMILES: O=C(NCc1nnc(-c2ccccc2)s1)c1cn([C@@H]2CO[C@H]3[C@@H]2OC[C@H]3O)nn1] | 1-((3R,3aR,6R,6aR)-6-hydroxyhexahydrofuro[3,2- b]furan-3-yl)-N-((5-phenyl-1,3,4-thiadiazol-2- yl)methyl)-1H-1,2,3-triazole-4-carboxamide |
| 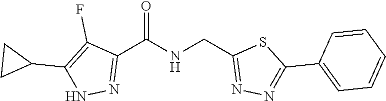 [SMILES: O=C(NCc1nnc(-c2ccccc2)s1)c1n[nH]c(C2CC2)c1F] | 5-cyclopropyl-4-fluoro-N-[(5-phenyl-1,3,4- thiadiazol-2-yl)methyl]-1H-pyrazole-3- carboxamide |
| 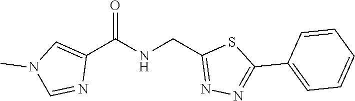 [SMILES: Cn1cnc(C(=O)NCc2nnc(-c3ccccc3)s2)c1] | 1-methyl-N-((5-phenyl-1,3,4-thiadiazol-2- yl)methyl)-1H-imidazole-4-carboxamide |
| 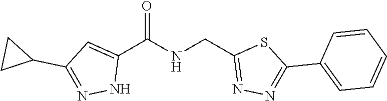 [SMILES: O=C(NCc1nnc(-c2ccccc2)s1)c1cc(C2CC2)n[nH]1] | 3-cyclopropyl-N-[(5-phenyl-1,3,4-thiadiazol-2- yl)methyl]-1H-pyrazole-5-carboxamide |
| 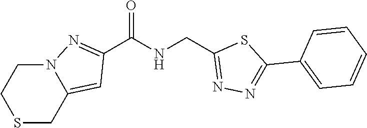 [SMILES: O=C(NCc1nnc(-c2ccccc2)s1)c1cc2n(n1)CCSC2] | N-[(5-phenyl-1,3,4-thiadiazol-2-yl)methyl]-6,7- dihydro-4H-pyrazolo[5,1-c][1,4]thiazine-2- carboxamide |
| 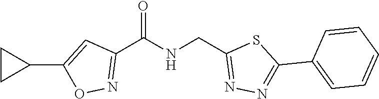 [SMILES: O=C(NCc1nnc(-c2ccccc2)s1)c1cc(C2CC2)on1] | 5-cyclopropyl-N-[(5-phenyl-1,3,4-thiadiazol-2- yl)methyl]isoxazole-3-carboxamide |
| 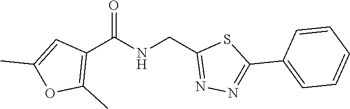 [SMILES: Cc1cc(C(=O)NCc2nnc(-c3ccccc3)s2)c(C)o1] | 2,5-dimethyl-N-[(5-phenyl-1,3,4-thiadiazol-2- yl)methyl]furan-3-carboxamide |
| 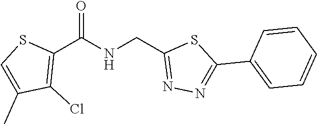 [SMILES: Cc1csc(C(=O)NCc2nnc(-c3ccccc3)s2)c1Cl] | 3-chloro-4-methyl-N-[(5-phenyl-1,3,4-thiadiazol-2- yl)methyl]thiophene-2-carboxamide |
| 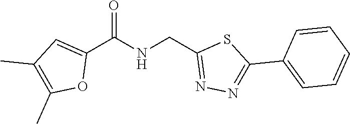 [SMILES: Cc1cc(C(=O)NCc2nnc(-c3ccccc3)s2)oc1C] | 4,5-dimethyl-N-[(5-phenyl-1,3,4-thiadiazol-2- yl)methyl]furan-2-carboxamide |
| 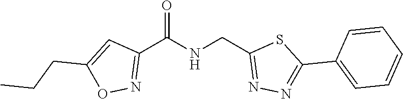 [SMILES: CCCc1cc(C(=O)NCc2nnc(-c3ccccc3)s2)no1] | N-[(5-phenyl-1,3,4-thiadiazol-2-yl)methyl]-5- propyl-isoxazole-3-carboxamide |
| 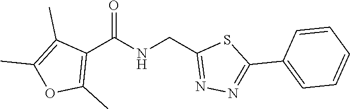 [SMILES: Cc1oc(C)c(C(=O)NCc2nnc(-c3ccccc3)s2)c1C] | 2,4,5-trimethyl-N-[(5-phenyl-1,3,4-thiadiazol-2- yl)methyl]furan-3-carboxamide |
| 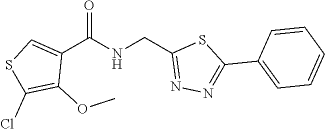 [SMILES: COc1c(C(=O)NCc2nnc(-c3ccccc3)s2)csc1Cl] | 5-chloro-4-methoxy-N-[(5-phenyl-1,3,4-thiadiazol- 2-yl)methyl]thiophene-3-carboxamide |
| 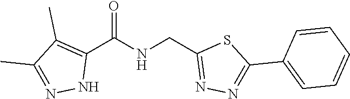 [SMILES: Cc1n[nH]c(C(=O)NCc2nnc(-c3ccccc3)s2)c1C] | 3,4-dimethyl-N-((5-phenyl-1,3,4-thiadiazol-2- yl)methyl)-1H-pyrazole-5-carboxamide |
| 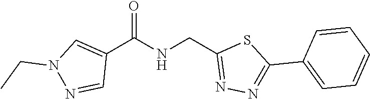 [SMILES: CCn1cc(C(=O)NCc2nnc(-c3ccccc3)s2)cn1] | 1-ethyl-N-[(5-phenyl-1,3,4-thiadiazol-2- yl)methyl]pyrazole-4-carboxamide |
| 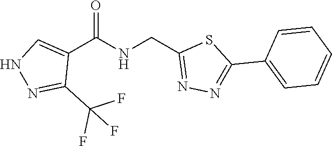 [SMILES: O=C(NCc1nnc(-c2ccccc2)s1)c1c[nH]nc1C(F)(F)F] | N-[(5-phenyl-1,3,4-thiadiazol-2-yl)methyl]-3- (trifluoromethyl)-1H-pyrazole-4-carboxamide |
| 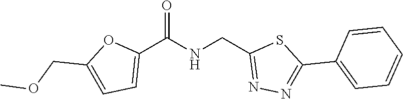 [SMILES: COCc1ccc(C(=O)NCc2nnc(-c3ccccc3)s2)o1] | 5-(methoxymethyl)-N-[(5-phenyl-1,3,4-thiadiazol- 2-yl)methyl]furan-2-carboxamide |
| 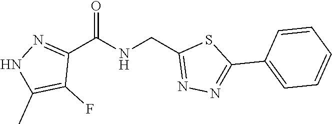 [SMILES: Cc1[nH]nc(C(=O)NCc2nnc(-c3ccccc3)s2)c1F] | 4-fluoro-5-methyl-N-[(5-phenyl-1,3,4-thiadiazol-2- yl)methyl]-1H-pyrazole-3-carboxamide |
| 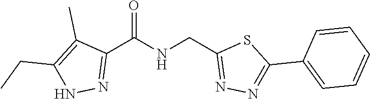 [SMILES: CCc1[nH]nc(C(=O)NCc2nnc(-c3ccccc3)s2)c1C] | 5-ethyl-4-methyl-N-((5-phenyl-1,3,4-thiadiazol-2- yl)methyl)-1H-pyrazole-3-carboxamide |
| 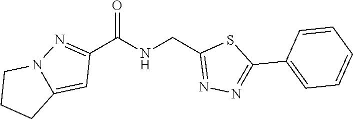 [SMILES: O=C(NCc1nnc(-c2ccccc2)s1)c1cc2n(n1)CCC2] | N-[(5-phenyl-1,3,4-thiadiazol-2-yl)methyl]-5,6- dihydro-4H-pyrrolo[1,2-b]pyrazole-2-carboxamide |
| 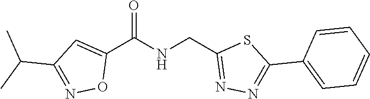 [SMILES: CC(C)c1cc(C(=O)NCc2nnc(-c3ccccc3)s2)on1] | 3-isopropyl-N-[(5-phenyl-1,3,4-thiadiazol-2- yl)methyl]isoxazole-5-carboxamide |
| 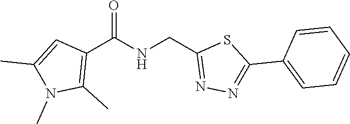 [SMILES: Cc1cc(C(=O)NCc2nnc(-c3ccccc3)s2)c(C)n1C] | 1,2,5-trimethyl-N-[(5-phenyl-1,3,4-thiadiazol-2- yl)methyl]pyrrole-3-carboxamide |
| 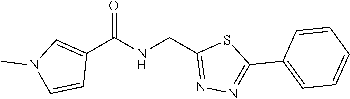 [SMILES: Cn1ccc(C(=O)NCc2nnc(-c3ccccc3)s2)c1] | 1-methyl-N-[(5-phenyl-1,3,4-thiadiazol-2- yl)methyl]pyrrole-3-carboxamide |
| 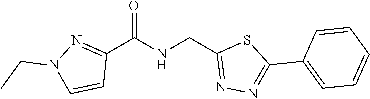 [SMILES: CCn1ccc(C(=O)NCc2nnc(-c3ccccc3)s2)n1] | 1-ethyl-N-[(5-phenyl-1,3,4-thiadiazol-2- yl)methyl]pyrazole-3-carboxamide |
| 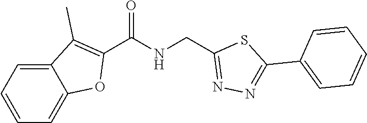 [SMILES: Cc1c(C(=O)NCc2nnc(-c3ccccc3)s2)oc2ccccc12] | 3-methyl-N-[(5-phenyl-1,3,4-thiadiazol-2- yl)methyl]benzofuran-2-carboxamide |
| 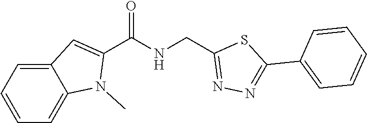 [SMILES: Cn1c(C(=O)NCc2nnc(-c3ccccc3)s2)cc2ccccc21] | 1-methyl-N-((5-phenyl-1,3,4-thiadiazol-2- yl)methyl)-1H-indole-2-carboxamide |
| 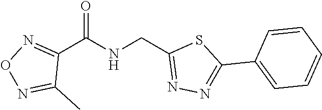 [SMILES: Cc1nonc1C(=O)NCc1nnc(-c2ccccc2)s1] | 4-methyl-N-[(5-phenyl-1,3,4-thiadiazol-2- yl)methyl]-1,2,5-oxadiazole-3-carboxamide |
| 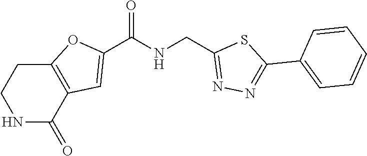 [SMILES: O=C(NCc1nnc(-c2ccccc2)s1)c1cc2c(o1)CCNC2=O] | 4-oxo-N-[(5-phenyl-1,3,4-thiadiazol-2-yl)methyl]- 6,7-dihydro-5H-furo[3,2-c]pyridine-2-carboxamide |
| 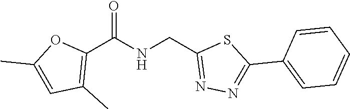 [SMILES: Cc1cc(C)c(C(=O)NCc2nnc(-c3ccccc3)s2)o1] | 3,5-dimethyl-N-[(5-phenyl-1,3,4-thiadiazol-2- yl)methyl]furan-2-carboxamide |
| 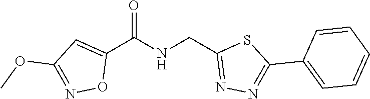 [SMILES: COc1cc(C(=O)NCc2nnc(-c3ccccc3)s2)on1] | 3-methoxy-N-[(5-phenyl-1,3,4-thiadiazol-2- yl)methyl]isoxazole-5-carboxamide |
| 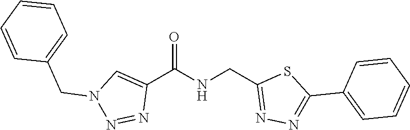 [SMILES: O=C(NCc1nnc(-c2ccccc2)s1)c1cn(Cc2ccccc2)nn1] | 1-benzyl-N-[(5-phenyl-1,3,4-thiadiazol-2- yl)methyl]triazole-4-carboxamide |
| 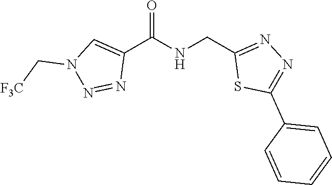 [SMILES: O=C(NCc1nnc(-c2ccccc2)s1)c1cn(CC(F)(F)F)nn1] | N-((5-phenyl-1,3,4-thiadiazol-2-yl)methyl)-1- (2,2,2-trifluoroethyl)-1H-1,2,3-triazole-4- carboxamide |
| 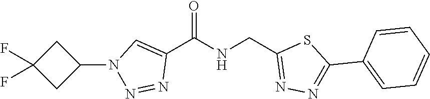 [SMILES: O=C(NCc1nnc(-c2ccccc2)s1)c1cn(C2CC(F)(F)C2)nn1] | 1-(3,3-difluorocyclobutyl)-N-((5-phenyl-1,3,4- thiadiazol-2-yl)methyl)-1H-1,2,3-triazole-4- carboxamide |
| 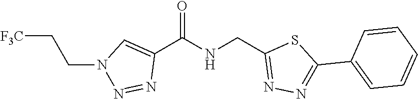 [SMILES: O=C(NCc1nnc(-c2ccccc2)s1)c1cn(CCC(F)(F)F)nn1] | N-((5-phenyl-1,3,4-thiadiazol-2-yl)methyl)-1- (3,3,3-trifluoropropyl)-1H-1,2,3-triazole-4- carboxamide |
| 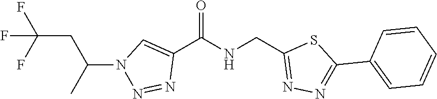 [SMILES: CC(CC(F)(F)F)n1cc(C(=O)NCc2nnc(-c3ccccc3)s2)nn1] | N-((5-phenyl-1,3,4-thiadiazol-2-yl)methyl)-1- (4,4,4-trifluorobutan-2-yl)-1H-1,2,3-triazole-4- carboxamide |
| 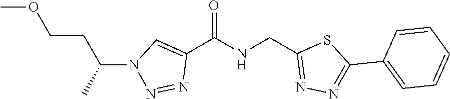 [SMILES: COCC[C@@H](C)n1cc(C(=O)NCc2nnc(-c3ccccc3)s2)nn1] | (R)-1-(4-methoxybutan-2-yl)-N-((5-phenyl-1,3,4- thiadiazol-2-yl)methyl)-1H-1,2,3-triazole-4- carboxamide |
| 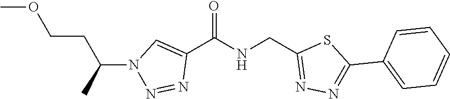 [SMILES: COCC[C@H](C)n1cc(C(=O)NCc2nnc(-c3ccccc3)s2)nn1] | (S)-1-(4-methoxybutan-2-yl)-N-((5-phenyl-1,3,4- thiadiazol-2-yl)methyl)-1H-1,2,3-triazole-4- carboxamide |
| 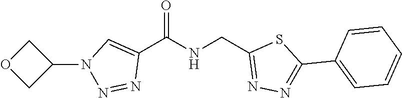 [SMILES: O=C(NCc1nnc(-c2ccccc2)s1)c1cn(C2COC2)nn1] | 1-(oxetan-3-yl)-N-((5-phenyl-1,3,4-thiadiazol-2- yl)methyl)-1H-1,2,3-triazole-4-carboxamide |
| 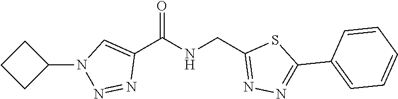 [SMILES: O=C(NCc1nnc(-c2ccccc2)s1)c1cn(C2CCC2)nn1] | 1-cyclobutyl-N-((5-phenyl-1,3,4-thiadiazol-2- yl)methyl)-1H-1,2,3-triazole-4-carboxamide |
| 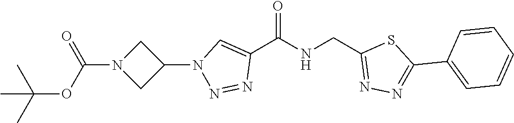 [SMILES: CC(C)(C)OC(=O)N1CC(n2cc(C(=O)NCc3nnc(-c4ccccc4)s3)nn2)C1] | tert-butyl 3-(4-(((5-phenyl-1,3,4-thiadiazol-2- yl)methyl)carbamoyl)-1H-1,2,3-triazol-1- yl)azetidine-1-carboxylate |
| 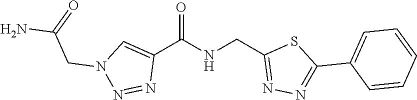 [SMILES: NC(=O)Cn1cc(C(=O)NCc2nnc(-c3ccccc3)s2)nn1] | 1-(2-amino-2-oxoethyl)-N-((5-phenyl-1,3,4- thiadiazol-2-yl)methyl)-1H-1,2,3-triazole-4- carboxamide |
| 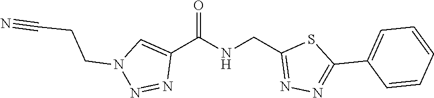 [SMILES: N#CCCn1cc(C(=O)NCc2nnc(-c3ccccc3)s2)nn1] | 1-(2-cyanoethyl)-N-((5-phenyl-1,3,4-thiadiazol-2- yl)methyl)-1H-1,2,3-triazole-4-carboxamide |
| 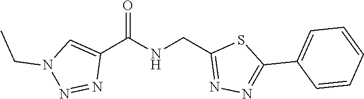 [SMILES: CCn1cc(C(=O)NCc2nnc(-c3ccccc3)s2)nn1] | 1-ethyl-N-((5-phenyl-1,3,4-thiadiazol-2-yl)methyl)- 1H-1,2,3-triazole-4-carboxamide |
| 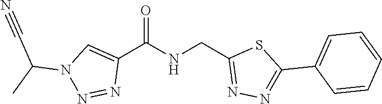 [SMILES: CC(C#N)n1cc(C(=O)NCc2nnc(-c3ccccc3)s2)nn1] | 1-(1-cyanoethyl)-N-((5-phenyl-1,3,4-thiadiazol-2- yl)methyl)-1H-1,2,3-triazole-4-carboxamide |
| 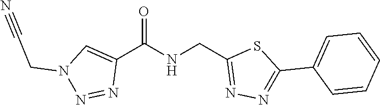 [SMILES: N#CCn1cc(C(=O)NCc2nnc(-c3ccccc3)s2)nn1] | 1-(cyanomethyl)-N-((5-phenyl-1,3,4-thiadiazol-2- yl)methyl)-1H-1,2,3-triazole-4-carboxamide |
| 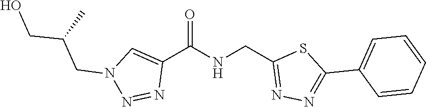 [SMILES: C[C@@H](CO)Cn1cc(C(=O)NCc2nnc(-c3ccccc3)s2)nn1] | (R)-1-(3-hydroxy-2-methylpropyl)-N-((5-phenyl- 1,3,4-thiadiazol-2-yl)methyl)-1H-1,2,3-triazole-4- carboxamide |
| 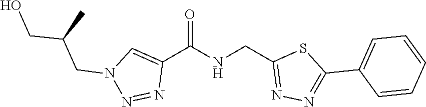 [SMILES: C[C@H](CO)Cn1cc(C(=O)NCc2nnc(-c3ccccc3)s2)nn1] | (S)-1-(3-hydroxy-2-methylpropyl)-N-((5-phenyl- 1,3,4-thiadiazol-2-yl)methyl)-1H-1,2,3-triazole-4- carboxamide |
| 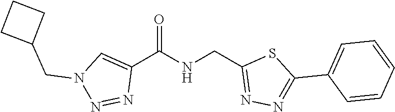 [SMILES: O=C(NCc1nnc(-c2ccccc2)s1)c1cn(CC2CCC2)nn1] | 1-(cyclobutylmethyl)-N-((5-phenyl-1,3,4- thiadiazol-2-yl)methyl)-1H-1,2,3-triazole-4- carboxamide |
| 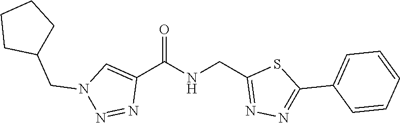 [SMILES: O=C(NCc1nnc(-c2ccccc2)s1)c1cn(CC2CCCC2)nn1] | 1-(cyclopentylmethyl)-N-((5-phenyl-1,3,4- thiadiazol-2-yl)methyl)-1H-1,2,3-triazole-4- carboxamide |
| 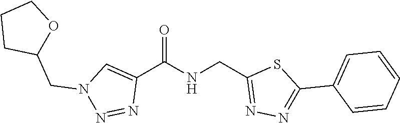 [SMILES: O=C(NCc1nnc(-c2ccccc2)s1)c1cn(CC2CCCO2)nn1] | N-((5-phenyl-1,3,4-thiadiazol-2-yl)methyl)-1- ((tetrahydrofuran-2-yl)methyl)-1H-1,2,3-triazole-4- carboxamide |
| 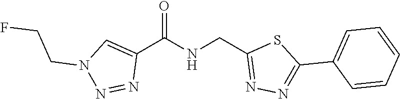 [SMILES: O=C(NCc1nnc(-c2ccccc2)s1)c1cn(CCF)nn1] | 1-(2-fluoroethyl)-N-((5-phenyl-1,3,4-thiadiazol-2- yl)methyl)-1H-1,2,3-triazole-4-carboxamide |
| 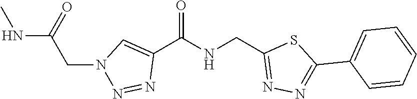 [SMILES: CNC(=O)Cn1cc(C(=O)NCc2nnc(-c3ccccc3)s2)nn1] | 1-(2-(methylamino)-2-oxoethyl)-N-((5-phenyl- 1,3,4-thiadiazol-2-yl)methyl)-1H-1,2,3-triazole-4- carboxamide |
| 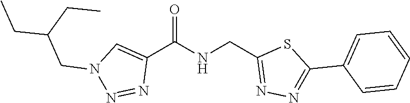 [SMILES: CCC(CC)Cn1cc(C(=O)NCc2nnc(-c3ccccc3)s2)nn1] | 1-(2-ethylbutyl)-N-((5-phenyl-1,3,4-thiadiazol-2- yl)methyl)-1H-1,2,3-triazole-4-carboxamide |
| 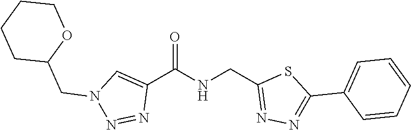 [SMILES: O=C(NCc1nnc(-c2ccccc2)s1)c1cn(CC2CCCCO2)nn1] | N-((5-phenyl-1,3,4-thiadiazol-2-yl)methyl)-1- ((tetrahydro-2H-pyran-2-yl)methyl)-1H-1,2,3- triazole-4-carboxamide |
| 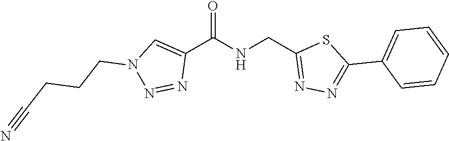 [SMILES: N#CCCCn1cc(C(=O)NCc2nnc(-c3ccccc3)s2)nn1] | 1-(3-cyanopropyl)-N-((5-phenyl-1,3,4-thiadiazol-2- yl)methyl)-1H-1,2,3-triazole-4-carboxamide |
| 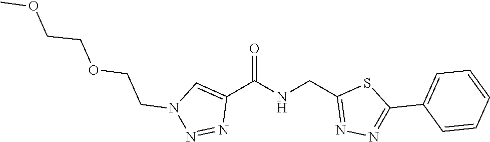 [SMILES: COCCOCCn1cc(C(=O)NCc2nnc(-c3ccccc3)s2)nn1] | 1-(2-(2-methoxyethoxy)ethyl)-N-((5-phenyl-1,3,4- thiadiazol-2-yl)methyl)-1H-1,2,3-triazole-4- carboxamide |
| 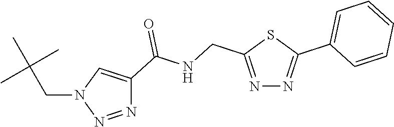 [SMILES: CC(C)(C)Cn1cc(C(=O)NCc2nnc(-c3ccccc3)s2)nn1] | 1-neopentyl-N-((5-phenyl-1,3,4-thiadiazol-2- yl)methyl)-1H-1,2,3-triazole-4-carboxamide |
| 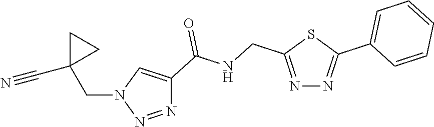 [SMILES: N#CC1(Cn2cc(C(=O)NCc3nnc(-c4ccccc4)s3)nn2)CC1] | 1-((1-cyanocyclopropyl)methyl)-N-((5-phenyl- 1,3,4-thiadiazol-2-yl)methyl)-1H-1,2,3-triazole-4- carboxamide |
| 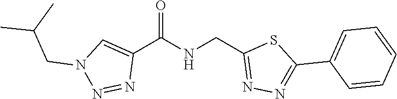 [SMILES: CC(C)Cn1cc(C(=O)NCc2nnc(-c3ccccc3)s2)nn1] | 1-isobutyl-N-((5-phenyl-1,3,4-thiadiazol-2- yl)methyl)-1H-1,2,3-triazole-4-carboxamide |
| 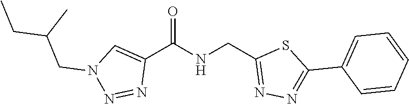 [SMILES: CCC(C)Cn1cc(C(=O)NCc2nnc(-c3ccccc3)s2)nn1] | 1-(2-methylbutyl)-N-((5-phenyl-1,3,4-thiadiazol-2- yl)methyl)-1H-1,2,3-triazole-4-carboxamide |
| 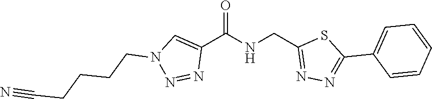 [SMILES: N#CCCCCn1cc(C(=O)NCc2nnc(-c3ccccc3)s2)nn1] | 1-(4-cyanobutyl)-N-((5-phenyl-1,3,4-thiadiazol-2- yl)methyl)-1H-1,2,3-triazole-4-carboxamide |
| 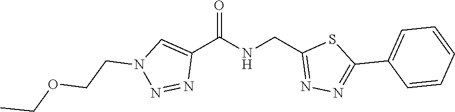 [SMILES: CCOCCn1cc(C(=O)NCc2nnc(-c3ccccc3)s2)nn1] | 1-(2-ethoxyethyl)-N-((5-phenyl-1,3,4-thiadiazol-2- yl)methyl)-1H-1,2,3-triazole-4-carboxamide |
| 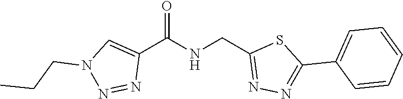 [SMILES: CCCn1cc(C(=O)NCc2nnc(-c3ccccc3)s2)nn1] | N-((5-phenyl-1,3,4-thiadiazol-2-yl)methyl)-1- propyl-1H-1,2,3-triazole-4-carboxamide |
| 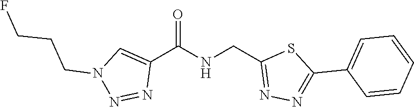 [SMILES: O=C(NCc1nnc(-c2ccccc2)s1)c1cn(CCCF)nn1] | 1-(3-fluoropropyl)-N-((5-phenyl-1,3,4-thiadiazol-2- yl)methyl)-1H-1,2,3-triazole-4-carboxamide |
| 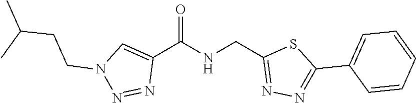 [SMILES: CC(C)CCn1cc(C(=O)NCc2nnc(-c3ccccc3)s2)nn1] | 1-isopentyl-N-((5-phenyl-1,3,4-thiadiazol-2- yl)methyl)-1H-1,2,3-triazole-4-carboxamide |
| 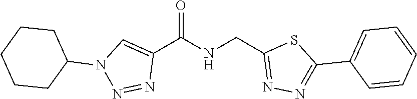 [SMILES: O=C(NCc1nnc(-c2ccccc2)s1)c1cn(C2CCCCC2)nn1] | 1-cyclohexyl-N-((5-phenyl-1,3,4-thiadiazol-2- yl)methyl)-1H-1,2,3-triazole-4-carboxamide |
| 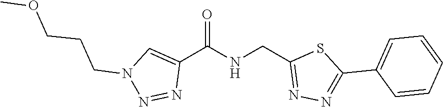 [SMILES: COCCCn1cc(C(=O)NCc2nnc(-c3ccccc3)s2)nn1] | 1-(3-methoxypropyl)-N-((5-phenyl-1,3,4- thiadiazol-2-yl)methyl)-1H-1,2,3-triazole-4- carboxamide |
| 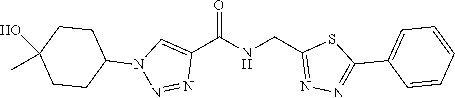 [SMILES: CC1(O)CCC(n2cc(C(=O)NCc3nnc(-c4ccccc4)s3)nn2)CC1] | 1-(4-hydroxy-4-methylcyclohexyl)-N-((5-phenyl- 1,3,4-thiadiazol-2-yl)methyl)-1H-1,2,3-triazole-4- carboxamide |
| 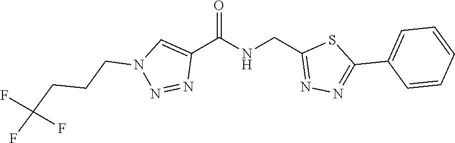 [SMILES: O=C(NCc1nnc(-c2ccccc2)s1)c1cn(CCCC(F)(F)F)nn1] | N-((5-phenyl-1,3,4-thiadiazol-2-yl)methyl)-1- (4,4,4-trifluorobutyl)-1H-1,2,3-triazole-4- carboxamide |
| 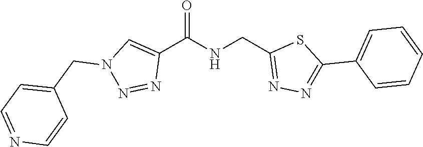 [SMILES: O=C(NCc1nnc(-c2ccccc2)s1)c1cn(Cc2ccncc2)nn1] | N-((5-phenyl-1,3,4-thiadiazol-2-yl)methyl)-1- (pyridin-4-ylmethyl)-1H-1,2,3-triazole-4- carboxamide |
| 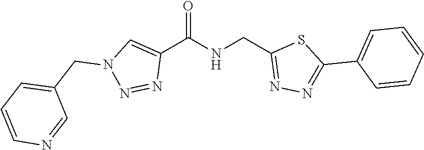 [SMILES: O=C(NCc1nnc(-c2ccccc2)s1)c1cn(Cc2cccnc2)nn1] | N-((5-phenyl-1,3,4-thiadiazol-2-yl)methyl)-1- (pyridin-3-ylmethyl)-1H-1,2,3-triazole-4- carboxamide |
| 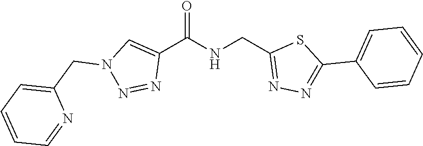 [SMILES: O=C(NCc1nnc(-c2ccccc2)s1)c1cn(Cc2ccccn2)nn1] | N-((5-phenyl-1,3,4-thiadiazol-2-yl)methyl)-1- (pyridin-2-ylmethyl)-1H-1,2,3-triazole-4- carboxamide |
| 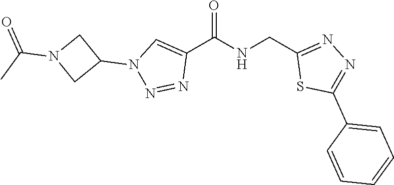 [SMILES: CC(=O)N1CC(n2cc(C(=O)NCc3nnc(-c4ccccc4)s3)nn2)C1] | 1-(1-acetylazetidin-3-yl)-N-((5-phenyl-1,3,4- thiadiazol-2-yl)methyl)-1H-1,2,3-triazole-4- carboxamide |
| 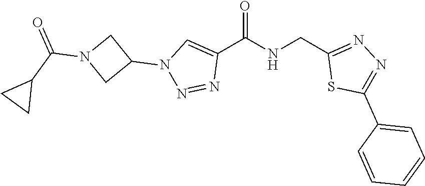 [SMILES: O=C(NCc1nnc(-c2ccccc2)s1)c1cn(C2CN(C(=O)C3CC3)C2)nn1] | 1-(1-(cyclopropanecarbonyl)azetidin-3-yl)-N-((5- phenyl-1,3,4-thiadiazol-2-yl)methyl)-1H-1,2,3- triazole-4-carboxamide |
| 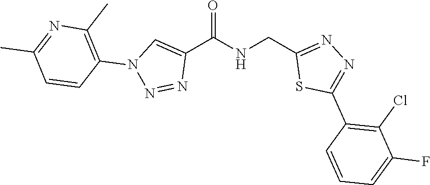 [SMILES: Cc1ccc(-n2cc(C(=O)NCc3nnc(-c4cccc(F)c4Cl)s3)nn2)c(C)n1] | N-((5-(3-chloro-2-fluorophenyl)-1,3,4-thiadiazol-2- yl)methyl)-1-(2,6-dimethylpyridin-3-yl)-1H-1,2,3- triazole-4-carboxamide |
| 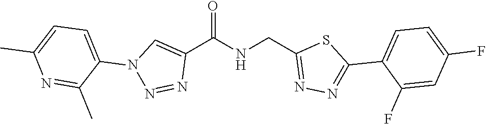 [SMILES: Cc1ccc(-n2cc(C(=O)NCc3nnc(-c4ccc(F)cc4F)s3)nn2)c(C)n1] | N-((5-(2,4-difluorophenyl)-1,3,4-thiadiazol-2- yl)methyl)-1-(2,6-dimethylpyridin-3-yl)-1H-1,2,3- triazole-4-carboxamide |
| 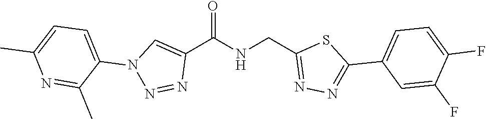 [SMILES: Cc1ccc(-n2cc(C(=O)NCc3nnc(-c4ccc(F)c(F)c4)s3)nn2)c(C)n1] | N-((5-(3,4-difluorophenyl)-1,3,4-thiadiazol-2- yl)methyl)-1-(2,6-dimethylpyridin-3-yl)-1H-1,2,3- triazole-4-carboxamide |
| 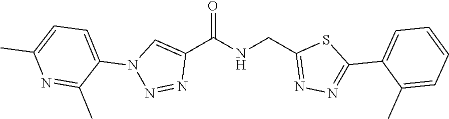 [SMILES: Cc1ccc(-n2cc(C(=O)NCc3nnc(-c4ccccc4C)s3)nn2)c(C)n1] | 1-(2,6-dimethylpyridin-3-yl)-N-((5-(o-tolyl)-1,3,4- thiadiazol-2-yl)methyl)-1H-1,2,3-triazole-4- carboxamide |
| 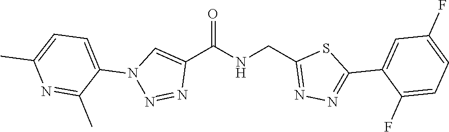 [SMILES: Cc1ccc(-n2cc(C(=O)NCc3nnc(-c4cc(F)ccc4F)s3)nn2)c(C)n1] | N-((5-(2,5-difluorophenyl)-1,3,4-thiadiazol-2- yl)methyl)-1-(2,6-dimethylpyridin-3-yl)-1H-1,2,3- triazole-4-carboxamide |
| 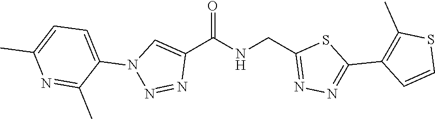 [SMILES: Cc1ccc(-n2cc(C(=O)NCc3nnc(-c4ccsc4C)s3)nn2)c(C)n1] | 1-(2,6-dimethylpyridin-3-yl)-N-((5-(2- methylthiophen-3-yl)-1,3,4-thiadiazol-2- yl)methyl)-1H-1,2,3-triazole-4-carboxamide |
| 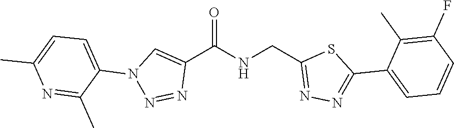 [SMILES: Cc1ccc(-n2cc(C(=O)NCc3nnc(-c4cccc(F)c4C)s3)nn2)c(C)n1] | 1-(2,6-dimethylpyridin-3-yl)-N-((5-(3-fluoro-2- methylphenyl)-1,3,4-thiadiazol-2-yl)methyl)-1H- 1,2,3-triazole-4-carboxamide |
| 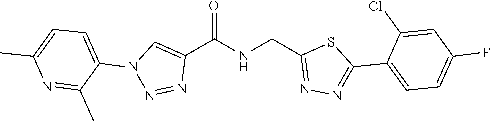 [SMILES: Cc1ccc(-n2cc(C(=O)NCc3nnc(-c4ccc(F)cc4Cl)s3)nn2)c(C)n1] | N-((5-(2-chloro-4-fluorophenyl)-1,3,4-thiadiazol-2- yl)methyl)-1-(2,6-dimethylpyridin-3-yl)-1H-1,2,3- triazole-4-carboxamide |
| 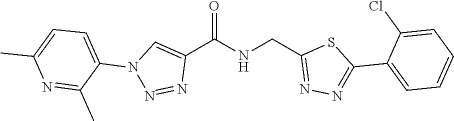 [SMILES: Cc1ccc(-n2cc(C(=O)NCc3nnc(-c4ccccc4Cl)s3)nn2)c(C)n1] | N-((5-(2-chlorophenyl)-1,3,4-thiadiazol-2- yl)methyl)-1-(2,6-dimethylpyridin-3-yl)-1H-1,2,3- triazole-4-carboxamide |
| 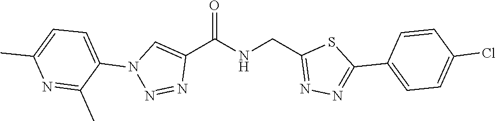 [SMILES: Cc1ccc(-n2cc(C(=O)NCc3nnc(-c4ccc(Cl)cc4)s3)nn2)c(C)n1] | N-((5-(4-chlorophenyl)-1,3,4-thiadiazol-2- yl)methyl)-1-(2,6-dimethylpyridin-3-yl)-1H-1,2,3- triazole-4-carboxamide |
| 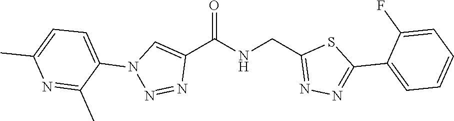 [SMILES: Cc1ccc(-n2cc(C(=O)NCc3nnc(-c4ccccc4F)s3)nn2)c(C)n1] | 1-(2,6-dimethylpyridin-3-yl)-N-((5-(2- fluorophenyl)-1,3,4-thiadiazol-2-yl)methyl)-1H- 1,2,3-triazole-4-carboxamide |
| 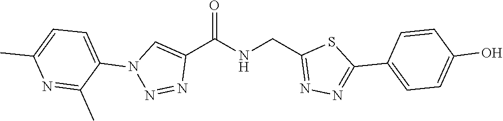 [SMILES: Cc1ccc(-n2cc(C(=O)NCc3nnc(-c4ccc(O)cc4)s3)nn2)c(C)n1] | 1-(2,6-dimethylpyridin-3-yl)-N-((5-(4- hydroxyphenyl)-1,3,4-thiadiazol-2-yl)methyl)-1H- 1,2,3-triazole-4-carboxamide |
| 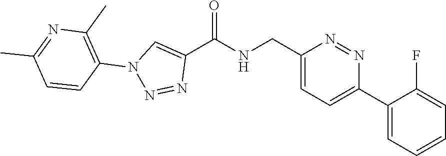 [SMILES: Cc1ccc(-n2cc(C(=O)NCc3ccc(-c4ccccc4F)nn3)nn2)c(C)n1] | Preparation of 1-(2,6-dimethylpyridin-3-yl)-N-((6- (2-fluorophenyl)pyridazin-3-yl)methyl)-1H-1,2,3- triazole-4-carboxamide |
| 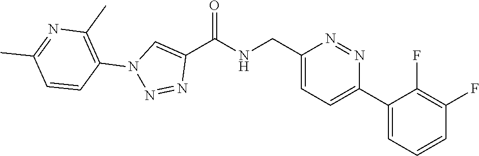 [SMILES: Cc1ccc(-n2cc(C(=O)NCc3ccc(-c4cccc(F)c4F)nn3)nn2)c(C)n1] | N-((6-(2,3-difluorophenyl)pyridazin-3-yl)methyl)- 1-(2,6-dimethylpyridin-3-yl)-1H-1,2,3-triazole-4- carboxamide |
| 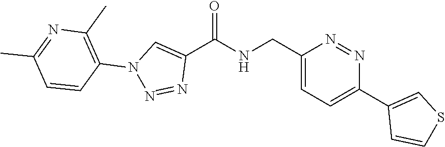 [SMILES: Cc1ccc(-n2cc(C(=O)NCc3ccc(-c4ccsc4)nn3)nn2)c(C)n1] | 1-(2,6-dimethylpyridin-3-yl)-N-((6-(thiophen-3- yl)pyridazin-3-yl)methyl)-1H-1,2,3-triazole-4- carboxamide |
| 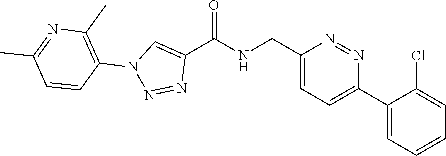 [SMILES: Cc1ccc(-n2cc(C(=O)NCc3ccc(-c4ccccc4Cl)nn3)nn2)c(C)n1] | N-((6-(2-chlorophenyl)pyridazin-3-yl)methyl)-1- (2,6-dimethylpyridin-3-yl)-1H-1,2,3-triazole-4- carboxamide |
| 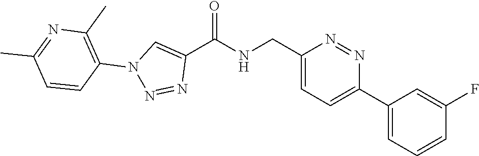 [SMILES: Cc1ccc(-n2cc(C(=O)NCc3ccc(-c4cccc(F)c4)nn3)nn2)c(C)n1] | 1-(2,6-dimethylpyridin-3-yl)-N-((6-(3- fluorophenyl)pyridazin-3-yl)methyl)-1H-1,2,3- triazole-4-carboxamide |
| 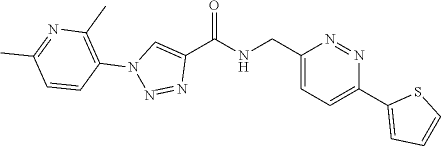 [SMILES: Cc1ccc(-n2cc(C(=O)NCc3ccc(-c4cccs4)nn3)nn2)c(C)n1] | 1-(2,6-dimethylpyridin-3-yl)-N-((6-(thiophen-2- yl)pyridazin-3-yl)methyl)-1H-1,2,3-triazole-4- carboxamide |
| 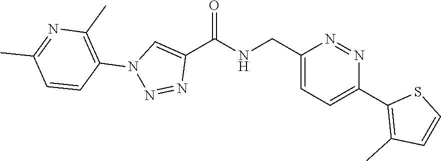 [SMILES: Cc1ccc(-n2cc(C(=O)NCc3ccc(-c4sccc4C)nn3)nn2)c(C)n1] | 1-(2,6-dimethylpyridin-3-yl)-N-((6-(3- methylthiophen-2-yl)pyridazin-3-yl)methyl)-1H- 1,2,3-triazole-4-carboxamide |
| 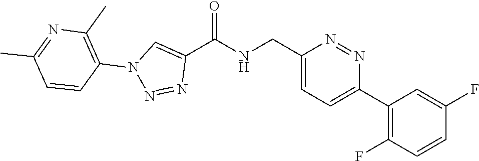 [SMILES: Cc1ccc(-n2cc(C(=O)NCc3ccc(-c4cc(F)ccc4F)nn3)nn2)c(C)n1] | N-((6-(2,5-difluorophenyl)pyridazin-3-yl)methyl)- 1-(2,6-dimethylpyridin-3-yl)-1H-1,2,3-triazole-4- carboxamide |
| 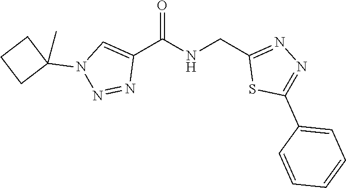 [SMILES: CC1(n2cc(C(=O)NCc3nnc(-c4ccccc4)s3)nn2)CCC1] | 1-(1-methylcyclobutyl)-N-((5-phenyl-1,3,4- thiadiazol-2-yl)methyl)-1H-1,2,3-triazole-4- carboxamide |
| 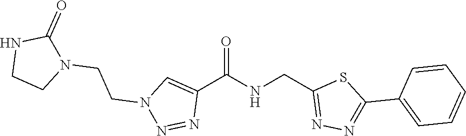 [SMILES: O=C(NCc1nnc(-c2ccccc2)s1)c1cn(CCN2CCNC2=O)nn1] | 1-(2-(2-oxoimidazolidin-1-yl)ethyl)-N-((5-phenyl- 1,3,4-thiadiazol-2-yl)methyl)-1H-1,2,3-triazole-4- carboxamide |
| 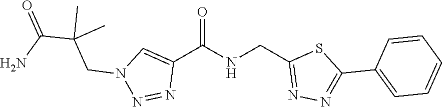 [SMILES: CC(C)(Cn1cc(C(=O)NCc2nnc(-c3ccccc3)s2)nn1)C(N)=O] | 1-(3-amino-2,2-dimethyl-3-oxo-propyl)-N-[(5- phenyl-1,3,4-thiadiazol-2-yl)methyl]triazole-4- carboxamide |
| 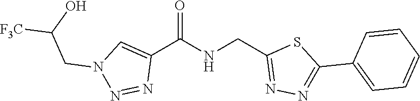 [SMILES: O=C(NCc1nnc(-c2ccccc2)s1)c1cn(CC(O)C(F)(F)F)nn1] | N-((5-phenyl-1,3,4-thiadiazol-2-yl)methyl)-1- (3,3,3-trifluoro-2-hydroxypropyl)-1H-1,2,3- triazole-4-carboxamide |
| 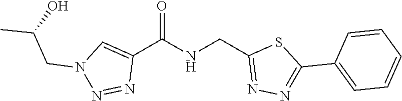 [SMILES: C[C@H](O)Cn1cc(C(=O)NCc2nnc(-c3ccccc3)s2)nn1] | (S)-1-(2-hydroxypropyl)-N-((5-phenyl-1,3,4- thiadiazol-2-yl)methyl)-1H-1,2,3-triazole-4- carboxamide |
| 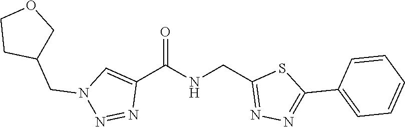 [SMILES: O=C(NCc1nnc(-c2ccccc2)s1)c1cn(CC2CCOC2)nn1] | N-((5-phenyl-1,3,4-thiadiazol-2-yl)methyl)-1- ((tetrahydrofuran-3-yl)methyl)-1H-1,2,3-triazole-4- carboxamide |
| 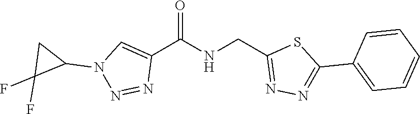 [SMILES: O=C(NCc1nnc(-c2ccccc2)s1)c1cn(C2CC2(F)F)nn1] | 1-(2,2-difluorocyclopropyl)-N-((5-phenyl-1,3,4- thiadiazol-2-yl)methyl)-1H-1,2,3-triazole-4- carboxamide |
| 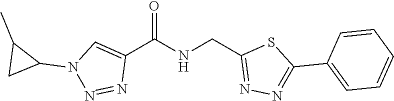 [SMILES: CC1CC1n1cc(C(=O)NCc2nnc(-c3ccccc3)s2)nn1] | Cis-1-(2-methylcyclopropyl)-N-((5-phenyl-1,3,4- thiadiazol-2-yl)methyl)-1H-1,2,3-triazole-4- carboxamide |
| 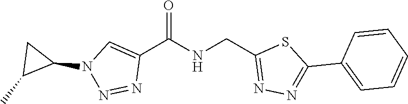 [SMILES: C[C@@H]1C[C@H]1n1cc(C(=O)NCc2nnc(-c3ccccc3)s2)nn1] | 1-((1R,2R)-2-methylcyclopropyl)-N-((5-phenyl- 1,3,4-thiadiazol-2-yl)methyl)-1H-1,2,3-triazole-4- carboxamide |
| 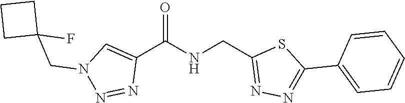 [SMILES: O=C(NCc1nnc(-c2ccccc2)s1)c1cn(CC2(F)CCC2)nn1] | 1-((1-fluorocyclobutyl)methyl)-N-((5-phenyl-1,3,4- thiadiazol-2-yl)methyl)-1H-1,2,3-triazole-4- carboxamide |
| 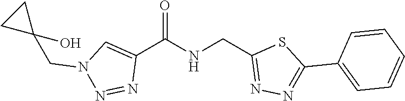 [SMILES: O=C(NCc1nnc(-c2ccccc2)s1)c1cn(CC2(O)CC2)nn1] | 1-((1-hydroxycyclopropyl)methyl)-N-((5-phenyl- 1,3,4-thiadiazol-2-yl)methyl)-1H-1,2,3-triazole-4- carboxamide |
| 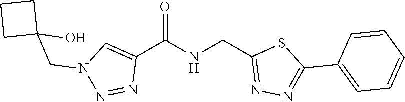 [SMILES: O=C(NCc1nnc(-c2ccccc2)s1)c1cn(CC2(O)CCC2)nn1] | 1-((1-hydroxycyclobutyl)methyl)-N-((5-phenyl- 1,3,4-thiadiazol-2-yl)methyl)-1H-1,2,3-triazole-4- carboxamide |
| 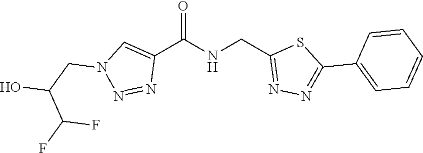 [SMILES: O=C(NCc1nnc(-c2ccccc2)s1)c1cn(CC(O)C(F)F)nn1] | 1-(3,3-difluoro-2-hydroxypropyl)-N-((5-phenyl- 1,3,4-thiadiazol-2-yl)methyl)-1H-1,2,3-triazole-4- carboxamide |
| 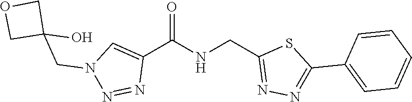 [SMILES: O=C(NCc1nnc(-c2ccccc2)s1)c1cn(CC2(O)COC2)nn1] | 1-((3-hydroxyoxetan-3-yl)methyl)-N-((5-phenyl- 1,3,4-thiadiazol-2-yl)methyl)-1H-1,2,3-triazole-4- carboxamide |
| 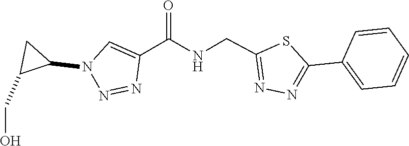 [SMILES: O=C(NCc1nnc(-c2ccccc2)s1)c1cn([C@@H]2C[C@H]2CO)nn1] | Trans-1-(2-(hydroxymethyl)cyclopropyl)-N-((5- phenyl-1,3,4-thiadiazol-2-yl)methyl)-1H-1,2,3- triazole-4-carboxamide |
| 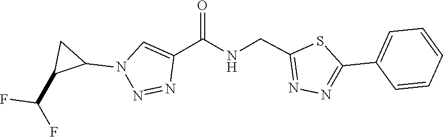 [SMILES: O=C(NCc1nnc(-c2ccccc2)s1)c1cn(C2C[C@@H]2C(F)F)nn1] | Trans-1-(-2-(difluoromethyl)cyclopropyl]-N-[(5- phenyl-1,3,4-thiadiazol-2-yl)methyl]-1H-1,2,3- triazole-4-carboxamide |
| 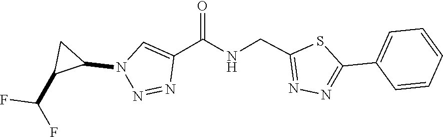 [SMILES: O=C(NCc1nnc(-c2ccccc2)s1)c1cn([C@@H]2C[C@@H]2C(F)F)nn1] | cis-1-(-2-(difluoromethyl)cyclopropyl]-N-[(5- phenyl-1,3,4-thiadiazol-2-yl)methyl]-1H-1,2,3- triazole-4-carboxamide |
| 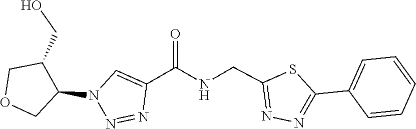 [SMILES: O=C(NCc1nnc(-c2ccccc2)s1)c1cn([C@H]2COC[C@@H]2CO)nn1] | 1-[(3S,4R)-4-(hydroxymethyl)oxolan-3-yl]-N-[(5- phenyl-1,3,4-thiadiazol-2-yl)methyl]-1H-1,2,3- triazole-4-carboxamide |
| 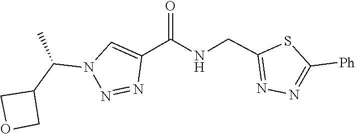 [SMILES: C[C@@H](C1COC1)n1cc(C(=O)NCc2nnc(-c3ccccc3)s2)nn1] | (S)-1-(1-(oxetan-3-yl)ethyl)-N-((5-phenyl-1,3,4- thiadiazol-2-yl)methyl)-1H-1,2,3-triazole-4- carboxamide |
| 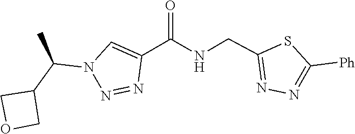 [SMILES: C[C@H](C1COC1)n1cc(C(=O)NCc2nnc(-c3ccccc3)s2)nn1] | (R)-1-(1-(oxetan-3-yl)ethyl)-N-((5-phenyl-1,3,4- thiadiazol-2-yl)methyl)-1H-1,2,3-triazole-4- carboxamide |
| 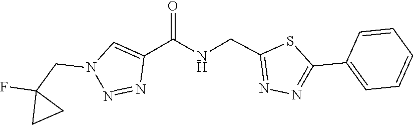 [SMILES: O=C(NCc1nnc(-c2ccccc2)s1)c1cn(CC2(F)CC2)nn1] | 1-((1-fluorocyclopropyl) methyl)-N-((5-phenyl-1,3,4-thiadiazol-2- yl)methyl)-1H-1,2,3-triazole-4-carboxamide |
| 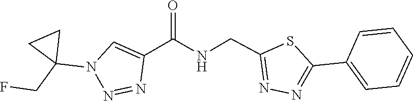 [SMILES: O=C(NCc1nnc(-c2ccccc2)s1)c1cn(C2(CF)CC2)nn1] | 1-(1-(fluoromethyl)cyclopropyl)-N-((5-phenyl- 1,3,4-thiadiazol-2-yl)methyl)-1H-1,2,3-triazole-4- carboxamide |
| 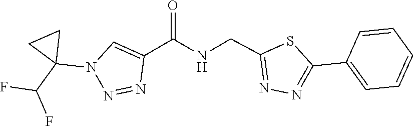 [SMILES: O=C(NCc1nnc(-c2ccccc2)s1)c1cn(C2(C(F)F)CC2)nn1] | 1-(1-(difluoromethyl)cyclopropyl)-N-((5-phenyl- 1,3,4-thiadiazol-2-yl)methyl)-1H-1,2,3-triazole-4- carboxamide |
| 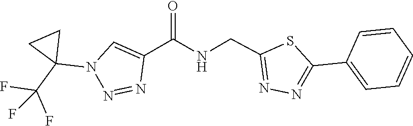 [SMILES: O=C(NCc1nnc(-c2ccccc2)s1)c1cn(C2(C(F)(F)F)CC2)nn1] | N-((5-phenyl-1,3,4-thiadiazol-2-yl)methyl)-1-(1- (trifluoromethyl)cyclopropyl)-1H-1,2,3-triazole-4- carboxamide |
| 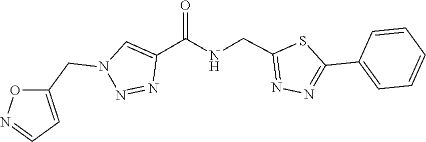 [SMILES: O=C(NCc1nnc(-c2ccccc2)s1)c1cn(Cc2ccno2)nn1] | 1-(isoxazol-5-ylmethyl)-N-((5-phenyl-1,3,4- thiadiazol-2-yl)methyl)-1H-1,2,3-triazole-4- carboxamide |
| 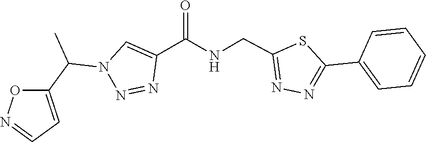 [SMILES: CC(c1ccno1)n1cc(C(=O)NCc2nnc(-c3ccccc3)s2)nn1] | 1-(1-(isoxazol-5-yl) ethyl)-N-((5-phenyl-1,3,4-thiadiazol-2-yl)methyl)- 1H-1,2,3-triazole-4-carboxamide |
| 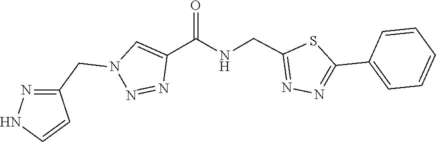 [SMILES: O=C(NCc1nnc(-c2ccccc2)s1)c1cn(Cc2cc[nH]n2)nn1] | 1-((1H-pyrazol-3-yl) methyl)-N-((5-phenyl-1,3,4-thiadiazol-2- yl)methyl)-1H-1,2,3-triazole-4-carboxamide |
| 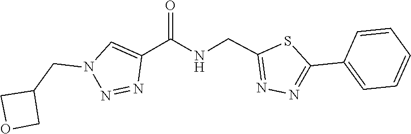 [SMILES: O=C(NCc1nnc(-c2ccccc2)s1)c1cn(CC2COC2)nn1] | 1-(oxetan-3-ylmethyl)-N-((5-phenyl-1,3,4- thiadiazol-2-yl)methyl)-1H-1,2,3-triazole-4- carboxamide |
| 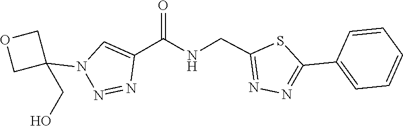 [SMILES: O=C(NCc1nnc(-c2ccccc2)s1)c1cn(C2(CO)COC2)nn1] | 1-(3-(hydroxymethyl)oxetan-3-yl)-N-((5-phenyl- 1,3,4-thiadiazol-2-yl)methyl)-1H- 1,2,3-triazole-4-carboxamide |
| 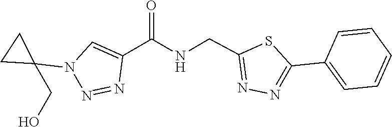 [SMILES: O=C(NCc1nnc(-c2ccccc2)s1)c1cn(C2(CO)CC2)nn1] | 1-(1-(hydroxymethyl)cyclopropyl)-N-((5-phenyl- 1,3,4-thiadiazol-2-yl)methyl)-1H- 1,2,3-triazole-4-carboxamide |
| 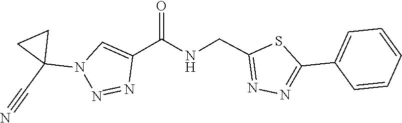 [SMILES: N#CC1(n2cc(C(=O)NCc3nnc(-c4ccccc4)s3)nn2)CC1] | 1-(1-cyanocyclopropyl)-N-((5-phenyl-1,3,4- thiadiazol-2-yl)methyl)-1H-1,2,3-triazole-4- carboxamide |
| 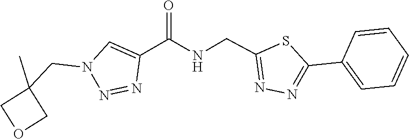 [SMILES: CC1(Cn2cc(C(=O)NCc3nnc(-c4ccccc4)s3)nn2)COC1] | 1-((3-methyloxetan-3-yl)methyl)-N-((5-phenyl- 1,3,4-thiadiazol-2-yl)methyl)-1H-1,2,3-triazole-4- carboxamide |
| 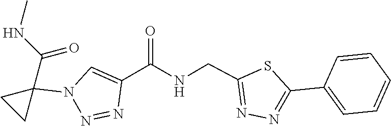 [SMILES: CNC(=O)C1(n2cc(C(=O)NCc3nnc(-c4ccccc4)s3)nn2)CC1] | 1-(1-(methylcarbamoyl)cyclopropyl)-N-((5-phenyl- 1,3,4-thiadiazol-2-yl)methyl)- 1H-1,2,3-triazole-4-carboxamide |
| 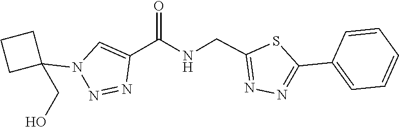 [SMILES: O=C(NCc1nnc(-c2ccccc2)s1)c1cn(C2(CO)CCC2)nn1] | 1-(1-(hydroxymethyl)cyclobutyl)-N-((5-phenyl- 1,3,4-thiadiazol-2-yl)methyl)-1H-1,2,3-triazole-4- carboxamide |
| 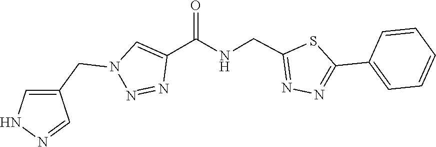 [SMILES: O=C(NCc1nnc(-c2ccccc2)s1)c1cn(Cc2cn[nH]c2)nn1] | 1-((1H-pyrazol-4-yl) methyl)-N-((5-phenyl-1,3,4-thiadiazol-2- yl)methyl)-1H-1,2,3-triazole-4-carboxamide |
| 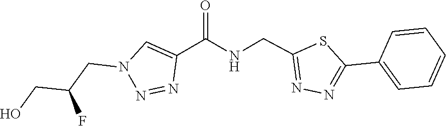 [SMILES: O=C(NCc1nnc(-c2ccccc2)s1)c1cn(C[C@@H](F)CO)nn1] | (R)-1-(2-fluoro-3-hydroxypropyl)-N-((5-phenyl- 1,3,4-thiadiazol-2-yl)methyl)-1H-1,2,3-triazole-4- carboxamide |
| 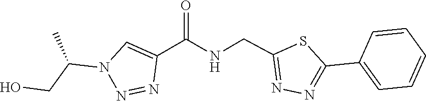 [SMILES: C[C@@H](CO)n1cc(C(=O)NCc2nnc(-c3ccccc3)s2)nn1] | (S)-1-(1-hydroxypropan-2-yl)-N-((5-phenyl-1,3,4- thiadiazol-2-yl)methyl)-1H-1,2,3-triazole-4- carboxamide |
| 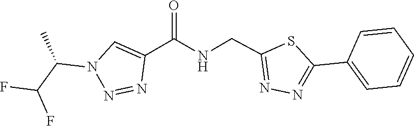 [SMILES: C[C@@H](C(F)F)n1cc(C(=O)NCc2nnc(-c3ccccc3)s2)nn1] | (S)-1-(1,1-difluoropropan-2-yl)-N-((5-phenyl- 1,3,4-thiadiazol-2-yl)methyl)-1H-1,2,3-triazole-4- carboxamide |
| 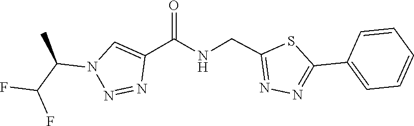 [SMILES: C[C@H](C(F)F)n1cc(C(=O)NCc2nnc(-c3ccccc3)s2)nn1] | (R)-1-(1,1-difluoropropan-2-yl)-N-((5-phenyl- 1,3,4-thiadiazol-2-yl)methyl)-1H-1,2,3-triazole-4- carboxamide |
| 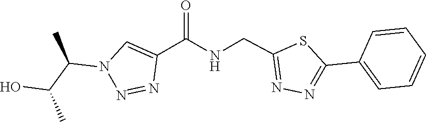 [SMILES: C[C@H](O)[C@@H](C)n1cc(C(=O)NCc2nnc(-c3ccccc3)s2)nn1] | 1-((2R,3S)-3-hydroxybutan-2-yl)-N-((5-phenyl- 1,3,4-thiadiazol-2-yl)methyl)-1H-1,2,3-triazole-4- carboxamide |
| 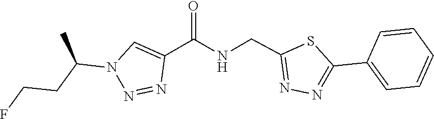 [SMILES: C[C@H](CCF)n1cc(C(=O)NCc2nnc(-c3ccccc3)s2)nn1] | (R)-1-(4-fluorobutan-2-yl)-N-((5-phenyl-1,3,4- thiadiazol-2-yl)methyl)-1H-1,2,3-triazole-4- carboxamide |
| 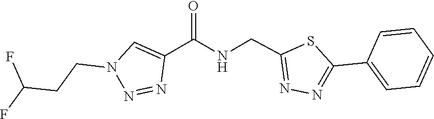 [SMILES: O=C(NCc1nnc(-c2ccccc2)s1)c1cn(CCC(F)F)nn1] | 1-(3,3-difluoropropyl)-N-((5-phenyl-1,3,4- thiadiazol-2-yl)methyl)-1H-1,2,3-triazole-4- carboxamide |
| 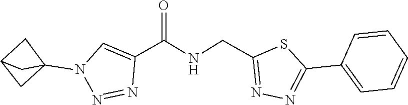 [SMILES: O=C(NCc1nnc(-c2ccccc2)s1)c1cn(C23CC(C2)C3)nn1] | 1-(bicyclo[1.1.1]pentan-1-yl)-N-((5-phenyl-1,3,4- thiadiazol-2-yl)methyl)-1H-1,2,3-triazole-4- carboxamide |
| 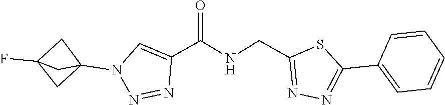 [SMILES: O=C(NCc1nnc(-c2ccccc2)s1)c1cn(C23CC(F)(C2)C3)nn1] | 1-(3-fluorobicyclo[1.1.1]pentan-1-yl)-N-((5- phenyl-1,3,4-thiadiazol-2-yl)methyl)-1H-1,2,3- triazole-4-carboxamide |
| 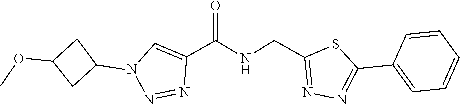 [SMILES: COC1CC(n2cc(C(=O)NCc3nnc(-c4ccccc4)s3)nn2)C1] | 1-(3-methoxycyclobutyl)-N-((5-phenyl-1,3,4- thiadiazol-2-yl)methyl)-1H-1,2,3-triazole-4- carboxamide |
| 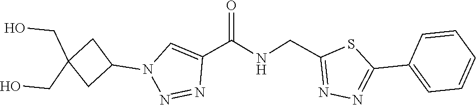 [SMILES: O=C(NCc1nnc(-c2ccccc2)s1)c1cn(C2CC(CO)(CO)C2)nn1] | Preparation of 1-(3,3- bis(hydroxymethyl)cyclobutyl)-N-((5-phenyl-1,3,4- thiadiazol-2-yl)methyl)-1H-1,2,3-triazole-4- carboxamide |
| 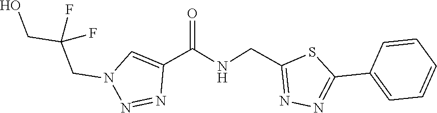 [SMILES: O=C(NCc1nnc(-c2ccccc2)s1)c1cn(CC(F)(F)CO)nn1] | 1-(2,2-difluoro-3-hydroxypropyl)-N-((5-phenyl- 1,3,4-thiadiazol-2-yl)methyl)-1H-1,2,3-triazole-4- carboxamide |
| 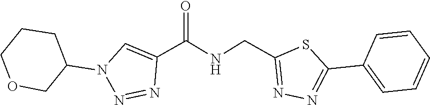 [SMILES: O=C(NCc1nnc(-c2ccccc2)s1)c1cn(C2CCCOC2)nn1] | N-((5-phenyl-1,3,4-thiadiazol-2-yl)methyl)-1- (tetrahydro-2H-pyran-3-yl)-1H-1,2,3-triazole-4- carboxamide |
| 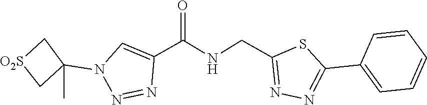 [SMILES: CC1(n2cc(C(=O)NCc3nnc(-c4ccccc4)s3)nn2)CS(=O)(=O)C1] | 1-(3-methyl-1,1-dioxidothietan-3-yl)-N-((5-phenyl- 1,3,4-thiadiazol-2-yl)methyl)-1H-1,2,3-triazole-4- carboxamide |
| 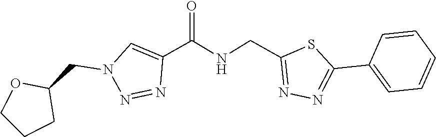 [SMILES: O=C(NCc1nnc(-c2ccccc2)s1)c1cn(C[C@H]2CCCO2)nn1] | (R)-N-((5-phenyl-1,3,4-thiadiazol-2-yl)methyl)-1- ((tetrahydrofuran-2-yl)methyl)-1H-1,2,3-triazole-4- carboxamide |
| 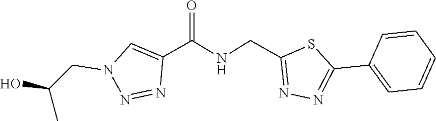 [SMILES: C[C@@H](O)Cn1cc(C(=O)NCc2nnc(-c3ccccc3)s2)nn1] | (R)-1-(2-hydroxypropyl)-N-((5-phenyl-1,3,4- thiadiazol-2-yl)methyl)-1H-1,2,3-triazole-4- carboxamide |
| 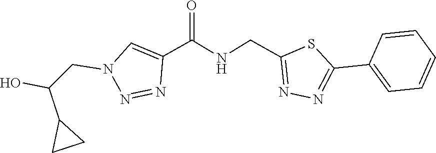 [SMILES: O=C(NCc1nnc(-c2ccccc2)s1)c1cn(CC(O)C2CC2)nn1] | 1-(2-cyclopropyl-2-hydroxyethyl)-N-((5-phenyl- 1,3,4-thiadiazol-2-yl)methyl)-1H-1,2,3-triazole-4- carboxamide |
| 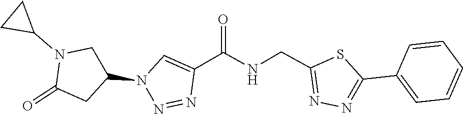 | (R)-1-(1-cyclopropyl-5-oxopyrrolidin-3-yl)-N-((5- phenyl-1,3,4-thiadiazol-2-yl)methyl)-1H-1,2,3- triazole-4-carboxamide |
| 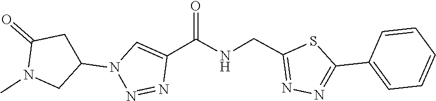 [SMILES: CN1CC(n2cc(C(=O)NCc3nnc(-c4ccccc4)s3)nn2)CC1=O] | 1-(1-methyl-5-oxopyrrolidin-3-yl)-N-((5-phenyl- 1,3,4-thiadiazol-2-yl)methyl)-1H-1,2,3-triazole-4- carboxamide |
| 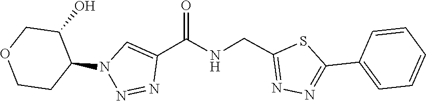 [SMILES: O=C(NCc1nnc(-c2ccccc2)s1)c1cn([C@H]2CCOC[C@@H]2O)nn1] | 1-((3R,4S)-3-hydroxytetrahydro-2H-pyran-4-yl)-N- ((5-phenyl-1,3,4-thiadiazol-2-yl)methyl)-1H-1,2,3- triazole-4-carboxamide |
| 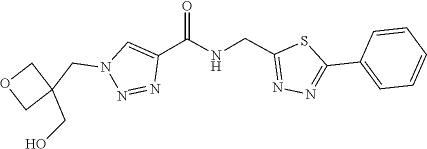 [SMILES: O=C(NCc1nnc(-c2ccccc2)s1)c1cn(CC2(CO)COC2)nn1] | 1-((3-(hydroxymethyl)oxetan-3-yl)methyl)-N-((5- phenyl-1,3,4-thiadiazol-2-yl)methyl)-1H-1,2,3- triazole-4-carboxamide |
| 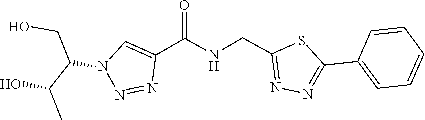 | 1-((2S,3S)-1,3-dihydroxybutan-2-yl)-N-((5-phenyl- 1,3,4-thiadiazol-2-yl)methyl)-1H-1,2,3-triazole-4- carboxamide |
| 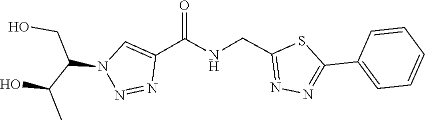 | 1-((2S,3R)-1,3-dihydroxybutan-2-yl)-N-((5-phenyl- 1,3,4-thiadiazol-2-yl)methyl)-1H-1,2,3-triazole-4- carboxamide |
| 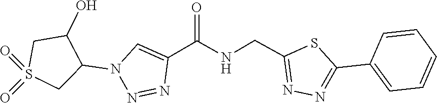 [SMILES: O=C(NCc1nnc(-c2ccccc2)s1)c1cn(C2CS(=O)(=O)CC2O)nn1] | 1-(4-hydroxy-1,1-dioxidotetrahydrothiophen-3-yl)- N-((5-phenyl-1,3,4-thiadiazol-2-yl)methyl)-1H- 1,2,3-triazole-4-carboxamide |
| 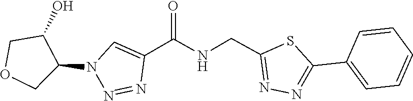 [SMILES: O=C(NCc1nnc(-c2ccccc2)s1)c1cn([C@H]2COC[C@@H]2O)nn1] | 1-((3S,4R)-4-hydroxytetrahydrofuran-3-yl)-N-((5- phenyl-1,3,4-thiadiazol-2-yl)methyl)-1H-1,2,3- triazole-4-carboxamide |
| 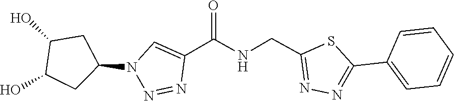 [SMILES: O=C(NCc1nnc(-c2ccccc2)s1)c1cn([C@@H]2C[C@@H](O)[C@@H](O)C2)nn1] | 1-((1s,3R,4S)-3,4-dihydroxycyclopentyl)-N-((5- phenyl-1,3,4-thiadiazol-2-yl)methyl)-1H-1,2,3- triazole-4-carboxamide |
| 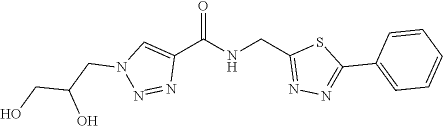 [SMILES: O=C(NCc1nnc(-c2ccccc2)s1)c1cn(CC(O)CO)nn1] | 1-(2,3-dihydroxypropyl)-N-((5-phenyl-1,3,4- thiadiazol-2-yl)methyl)-1H-1,2,3-triazole-4- carboxamide |
| 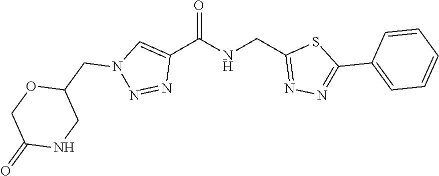 [SMILES: O=C1COC(Cn2cc(C(=O)NCc3nnc(-c4ccccc4)s3)nn2)CN1] | 1-((5-oxomorpholin-2-yl)methyl)-N-((5-phenyl- 1,3,4-thiadiazol-2-yl)methyl)-1H-1,2,3-triazole-4- carboxamide |
| 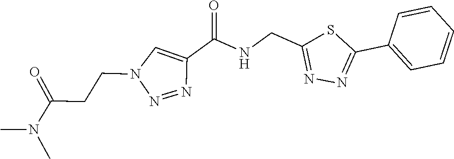 [SMILES: CN(C)C(=O)CCn1cc(C(=O)NCc2nnc(-c3ccccc3)s2)nn1] | 1-(3-(dimethylamino)-3-oxopropyl)-N-((5-phenyl- 1,3,4-thiadiazol-2-yl)methyl)-1H-1,2,3-triazole-4- carboxamide |
| 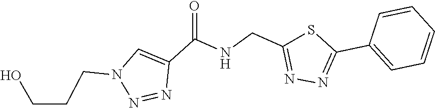 [SMILES: O=C(NCc1nnc(-c2ccccc2)s1)c1cn(CCCO)nn1] | 1-(3-hydroxypropyl)-N-((5-phenyl-1,3,4-thiadiazol- 2-yl)methyl)-1H-1,2,3-triazole-4-carboxamide |
| 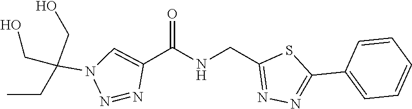 [SMILES: CCC(CO)(CO)n1cc(C(=O)NCc2nnc(-c3ccccc3)s2)nn1] | 1-(1-hydroxy-2-(hydroxymethyl)butan-2-yl)-N-((5- phenyl-1,3,4-thiadiazol-2-yl)methyl)-1H-1,2,3- triazole-4-carboxamide |
| 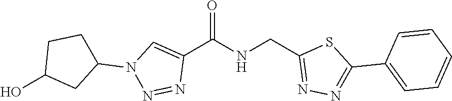 [SMILES: O=C(NCc1nnc(-c2ccccc2)s1)c1cn(C2CCC(O)C2)nn1] | 1-(3-hydroxycyclopentyl)-N-((5-phenyl-1,3,4- thiadiazol-2-yl)methyl)-1H-1,2,3-triazole-4- carboxamide |
| 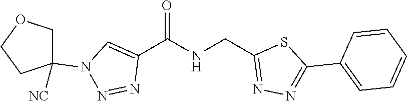 [SMILES: N#CC1(n2cc(C(=O)NCc3nnc(-c4ccccc4)s3)nn2)CCOC1] | 1-(3-cyanotetrahydrofuran-3-yl)-N-((5-phenyl- 1,3,4-thiadiazol-2-yl)methyl)-1H-1,2,3-triazole-4- carboxamide |
| 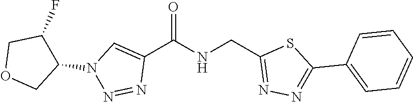 [SMILES: O=C(NCc1nnc(-c2ccccc2)s1)c1cn([C@@H]2COC[C@@H]2F)nn1] | 1-((3R,4R)-4-fluorotetrahydrofuran-3-yl)-N-((5- phenyl-1,3,4-thiadiazol-2-yl)methyl)-1H-1,2,3- triazole-4-carboxamide |
| 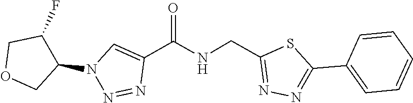 [SMILES: O=C(NCc1nnc(-c2ccccc2)s1)c1cn([C@H]2COC[C@@H]2F)nn1] | 1-((3S,4R)-4-fluorotetrahydrofuran-3-yl)-N-((5- phenyl-1,3,4-thiadiazol-2-yl)methyl)-1H-1,2,3- triazole-4-carboxamide |
| 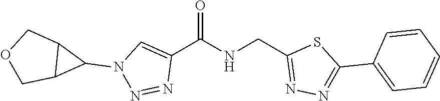 [SMILES: O=C(NCc1nnc(-c2ccccc2)s1)c1cn(C2C3COCC32)nn1] | 1-(3-oxabicyclo[3.1.0]hexan-6-yl)-N-((5-phenyl- 1,3,4-thiadiazol-2-yl)methyl)-1H-1,2,3-triazole-4- carboxamide |
| 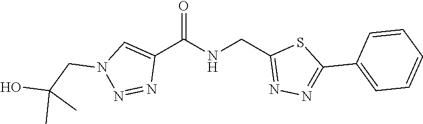 [SMILES: CC(C)(O)Cn1cc(C(=O)NCc2nnc(-c3ccccc3)s2)nn1] | 1-(2-hydroxy-2-methylpropyl)-N-((5-phenyl-1,3,4- thiadiazol-2-yl)methyl)-1H-1,2,3-triazole-4- carboxamide |
| 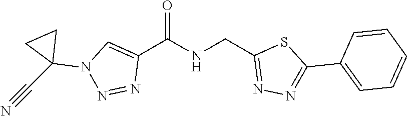 [SMILES: N#CC1(n2cc(C(=O)NCc3nnc(-c4ccccc4)s3)nn2)CC1] | 1-(1-cyanocyclopropyl)-N-((5-phenyl-1,3,4- thiadiazol-2-yl)methyl)-1H-1,2,3-triazole-4- carboxamide |
| 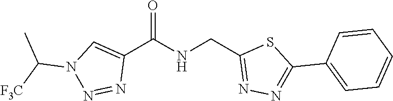 [SMILES: CC(n1cc(C(=O)NCc2nnc(-c3ccccc3)s2)nn1)C(F)(F)F] | N-((5-phenyl-1,3,4-thiadiazol-2-yl)methyl)-1- (1,1,1-trifluoropropan-2-yl)-1H-1,2,3-triazole-4- carboxamide |
| 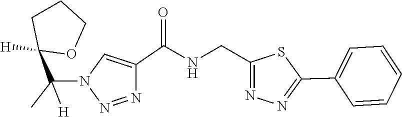 [SMILES: CC([C@H]1CCCO1)n1cc(C(=O)NCc2nnc(-c3ccccc3)s2)nn1] | N-((5-phenyl-1,3,4-thiadiazol-2-yl)methyl)-1-(1- ((R)-tetrahydrofuran-2-yl)ethyl)-1H-1,2,3-triazole- 4-carboxamide |
| 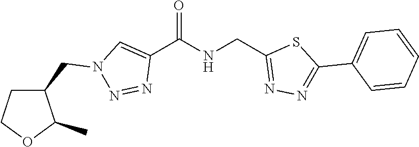 [SMILES: C[C@H]1OCC[C@H]1Cn1cc(C(=O)NCc2nnc(-c3ccccc3)s2)nn1] | 1-(((2R,3S)-2-methyltetrahydrofuran-3-yl)methyl)- N-((5-phenyl-1,3,4-thiadiazol-2-yl)methyl)-1H- 1,2,3-triazole-4-carboxamide |
| 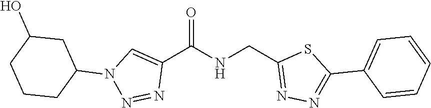 [SMILES: O=C(NCc1nnc(-c2ccccc2)s1)c1cn(C2CCCC(O)C2)nn1] | 1-(3-hydroxycyclohexyl)-N-((5-phenyl-1,3,4- thiadiazol-2-yl)methyl)-1H-1,2,3-triazole-4- carboxamide |
| 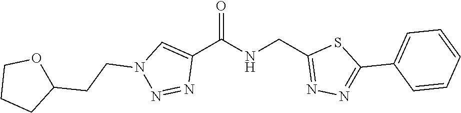 [SMILES: O=C(NCc1nnc(-c2ccccc2)s1)c1cn(CCC2CCCO2)nn1] | N-((5-phenyl-1,3,4-thiadiazol-2-yl)methyl)-1-(2- (tetrahydrofuran-2-yl)ethyl)-1H-1,2,3-triazole-4- carboxamide |
| 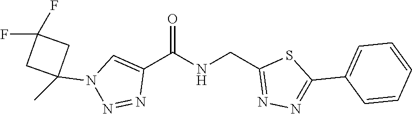 [SMILES: CC1(n2cc(C(=O)NCc3nnc(-c4ccccc4)s3)nn2)CC(F)(F)C1] | 1-(3,3-difluoro-1-methylcyclobutyl)-N-((5-phenyl- 1,3,4-thiadiazol-2-yl)methyl)-1H-1,2,3-triazole-4- carboxamide |
| 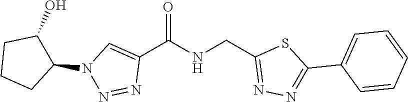 [SMILES: O=C(NCc1nnc(-c2ccccc2)s1)c1cn([C@H]2CCC[C@@H]2O)nn1] | 1-((1S,2S)-2-hydroxycyclopentyl)-N-((5-phenyl- 1,3,4-thiadiazol-2-yl)methyl)-1H-1,2,3-triazole-4- carboxamide |
| 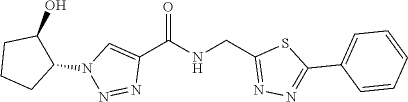 [SMILES: O=C(NCc1nnc(-c2ccccc2)s1)c1cn([C@@H]2CCC[C@H]2O)nn1] | 1-((1R,2R)-2-hydroxycyclopentyl)-N-((5-phenyl- 1,3,4-thiadiazol-2-yl)methyl)-1H-1,2,3-triazole-4- carboxamide |
| 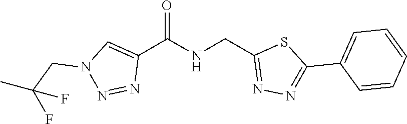 | 1-(2,2-difluoropropyl)-N-((5-phenyl-1,3,4- thiadiazol-2-yl)methyl)-1H-1,2,3-triazole-4- carboxamide |
| 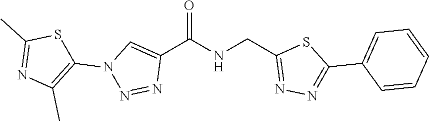 [SMILES: Cc1nc(C)c(-n2cc(C(=O)NCc3nnc(-c4ccccc4)s3)nn2)s1] | 1-(2,4-dimethylthiazol-5-yl)-N-((5-phenyl-1,3,4- thiadiazol-2-yl)methyl)-1H-1,2,3-triazole-4- carboxamide |
| 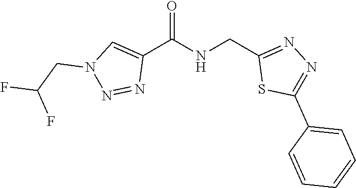 [SMILES: O=C(NCc1nnc(-c2ccccc2)s1)c1cn(CC(F)F)nn1] | Preparation of N-((5-phenyl-1,3,4-thiadiazol-2- yl)methyl)-1-(2,2,2-trifluoroethyl)-1H-1,2,3- triazole-4-carboxamide |
| 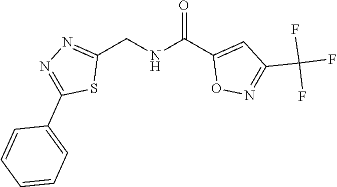 [SMILES: O=C(NCc1nnc(-c2ccccc2)s1)c1cc(C(F)(F)F)no1] | N-[(5-phenyl-1,3,4-thiadiazol-2-yl)methyl]-3- (trifluoromethyl)-1,2-oxazole-5-carboxamide |
| 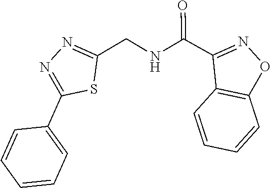 [SMILES: O=C(NCc1nnc(-c2ccccc2)s1)c1noc2ccccc12] | N-[(5-phenyl-1,3,4-thiadiazol-2-yl)methyl]-1,2- benzoxazole-3-carboxamide |
| 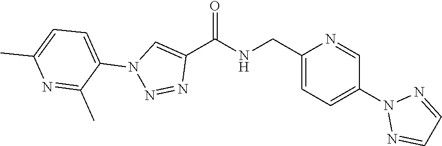 [SMILES: Cc1ccc(-n2cc(C(=O)NCc3ccc(-n4nccn4)cn3)nn2)c(C)n1] | 1-(2,6-dimethylpyridin-3-yl)-N-{[5-(2H-1,2,3- triazol-2-yl)pyridin-2-yl]methyl}-1H-1,2,3-triazole- 4-carboxamide |
| 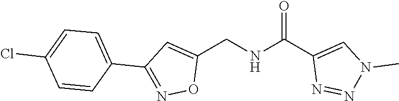 [SMILES: Cn1cc(C(=O)NCc2cc(-c3ccc(Cl)cc3)no2)nn1] | N-{[3-(4-chlorophenyl)-1,2-oxazol-5-yl]methyl}-1- methyl-1H-1,2,3-triazole-4-carboxamide |
| 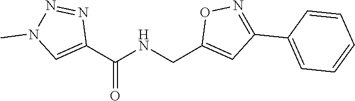 [SMILES: Cn1cc(C(=O)NCc2cc(-c3ccccc3)no2)nn1] | 1-methyl-N-[(3-phenyl-1,2-oxazol-5-yl)methyl]- 1H-1,2,3-triazole-4-carboxamide |
| 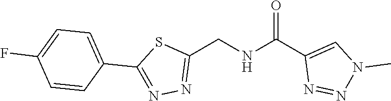 [SMILES: Cn1cc(C(=O)NCc2nnc(-c3ccc(F)cc3)s2)nn1] | N-{[5-(4-fluorophenyl)-1,3,4-thiadiazol-2- yl]methyl}-1-methyl-1H-1,2,3-triazole-4- carboxamide |
| 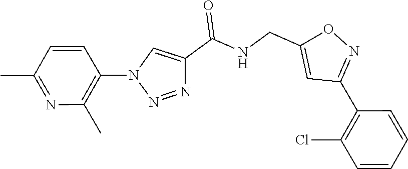 [SMILES: Cc1ccc(-n2cc(C(=O)NCc3cc(-c4ccccc4Cl)no3)nn2)c(C)n1] | N-{[3-(2-chlorophenyl)-1,2-oxazol-5-yl]methyl}-1- (2,6-dimethylpyridin-3-yl)-1H-1,2,3-triazole-4- carboxamide |
| 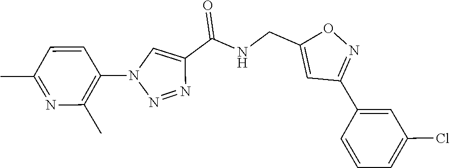 [SMILES: Cc1ccc(-n2cc(C(=O)NCc3cc(-c4cccc(Cl)c4)no3)nn2)c(C)n1] | N-{[3-(3-chlorophenyl)-1,2-oxazol-5-yl]methyl}-1- (2,6-dimethylpyridin-3-yl)-1H-1,2,3-triazole-4- carboxamide |
| 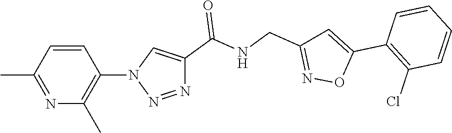 [SMILES: Cc1ccc(-n2cc(C(=O)NCc3cc(-c4ccccc4Cl)on3)nn2)c(C)n1] | N-{[5-(2-chlorophenyl)-1,2-oxazol-3-yl]methyl}-1- (2,6-dimethylpyridin-3-yl)-1H-1,2,3-triazole-4- carboxamide |
| 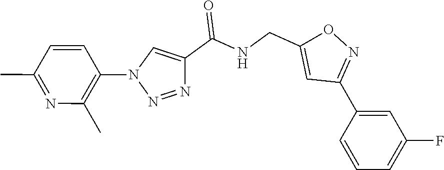 [SMILES: Cc1ccc(-n2cc(C(=O)NCc3cc(-c4cccc(F)c4)no3)nn2)c(C)n1] | 1-(2,6-dimethylpyridin-3-yl)-N-{[3-(3- fluorophenyl)-1,2-oxazol-5-yl]methyl}-1H-1,2,3- triazole-4-carboxamide |
| 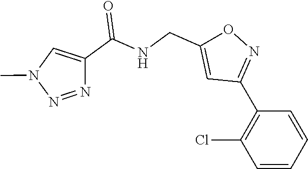 [SMILES: Cn1cc(C(=O)NCc2cc(-c3ccccc3Cl)no2)nn1] | N-{[3-(2-chlorophenyl)-1,2-oxazol-5-yl]methyl}-1- methyl-1H-1,2,3-triazole-4-carboxamide |
| 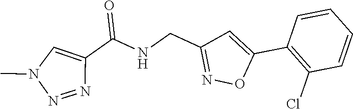 [SMILES: Cn1cc(C(=O)NCc2cc(-c3ccccc3Cl)on2)nn1] | N-{[5-(2-chlorophenyl)-1,2-oxazol-3-yl]methyl}-1- methyl-1H-1,2,3-triazole-4-carboxamide |
| 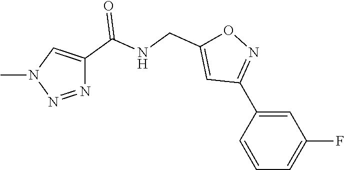 [SMILES: Cn1cc(C(=O)NCc2cc(-c3cccc(F)c3)no2)nn1] | N-{[3-(3-fluorophenyl)-1,2-oxazol-5-yl]methyl}-1- methyl-1H-1,2,3-triazole-4-carboxamide |
| 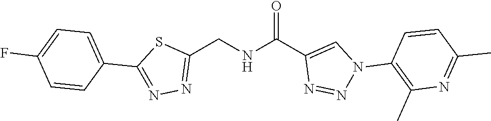 [SMILES: Cc1ccc(-n2cc(C(=O)NCc3nnc(-c4ccc(F)cc4)s3)nn2)c(C)n1] | 1-(2,6-dimethylpyridin-3-yl)-N-{[5-(4- fluorophenyl)-1,3,4-thiadiazol-2-yl]methyl}-1H- 1,2,3-triazole-4-carboxamide |
| 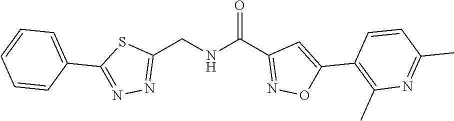 [SMILES: Cc1ccc(-c2cc(C(=O)NCc3nnc(-c4ccccc4)s3)no2)c(C)n1] | 5-(2,6-dimethylpyridin-3-yl)-N-[(5-phenyl-1,3,4- thiadiazol-2-yl)methyl]-1,2-oxazole-3-carboxamide |
| 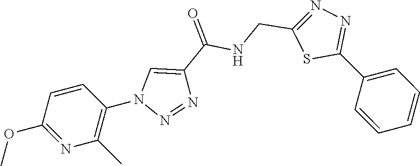 [SMILES: COc1ccc(-n2cc(C(=O)NCc3nnc(-c4ccccc4)s3)nn2)c(C)n1] | 1-(6-methoxy-2-methylpyridin-3-yl)- N-((5-phenyl-1,3,4-thiadiazol-2-yl)methyl)-1H- 1,2,3-triazole-4-carboxamide |
| 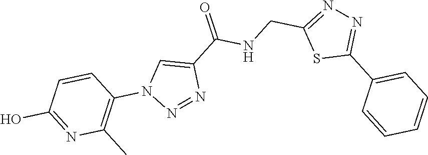 [SMILES: Cc1nc(O)ccc1-n1cc(C(=O)NCc2nnc(-c3ccccc3)s2)nn1] | 1-(6-hydroxy-2-methylpyridin-3-yl)-N- ((5-phenyl-1,3,4-thiadiazol-2-yl)methyl)- 1H-1,2,3-triazole-4-carboxamide |
| 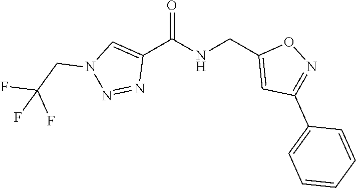 | N-((3-phenylisoxazol-5-yl)methyl)-1-(2,2,2- trifluoroethyl)-1H-1,2,3-triazole-4-carboxamide |
| 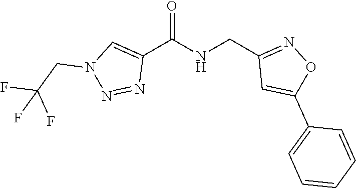 [SMILES: O=C(NCc1cc(-c2ccccc2)on1)c1cn(CC(F)(F)F)nn1] | N-((5-phenylisoxazol-3-yl)methyl)-1-(2,2,2- trifluoroethyl)-1H-1,2,3-triazole-4-carboxamide |
| 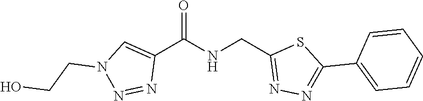 [SMILES: O=C(NCc1nnc(-c2ccccc2)s1)c1cn(CCO)nn1] | 1-(2-hydroxyethyl)-N-((5-phenyl-1,3,4-thiadiazol- 2-yl)methyl)-1H-1,2,3-triazole-4-carboxamide |
| 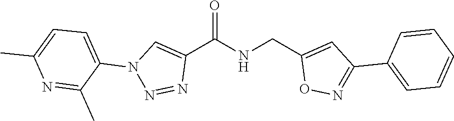 [SMILES: Cc1ccc(-n2cc(C(=O)NCc3cc(-c4ccccc4)no3)nn2)c(C)n1] | 1-(2,6-dimethylpyridin-3-yl)-N-((3-phenylisoxazol- 5-yl)methyl)-1H-1,2,3-triazole-4-carboxamide |
| 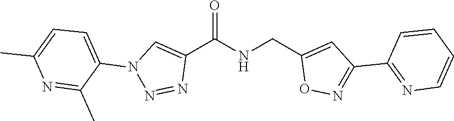 [SMILES: Cc1ccc(-n2cc(C(=O)NCc3cc(-c4ccccn4)no3)nn2)c(C)n1] | 1-(2,6-dimethylpyridin-3-yl)-N-((3-(pyridin-2- yl)isoxazol-5-yl)methyl)-1H-1,2,3-triazole-4- carboxamide |
| 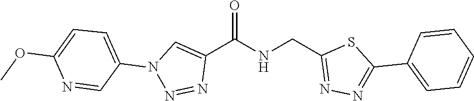 [SMILES: COc1ccc(-n2cc(C(=O)NCc3nnc(-c4ccccc4)s3)nn2)cn1] | 1-(6-methoxypyridin-3-yl)-N-((5-phenyl-1,3,4- thiadiazol-2-yl)methyl)-1H-1,2,3-triazole-4- carboxamide |
| 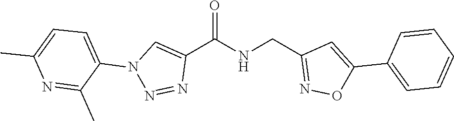 [SMILES: Cc1ccc(-n2cc(C(=O)NCc3cc(-c4ccccc4)on3)nn2)c(C)n1] | 1-(2,6-dimethylpyridin-3-yl)-N-((5-phenylisoxazol- 3-yl)methyl)-1H-1,2,3-triazole-4-carboxamide |
| 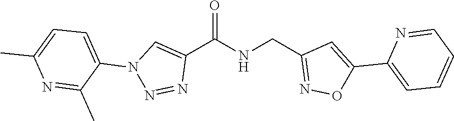 [SMILES: Cc1ccc(-n2cc(C(=O)NCc3cc(-c4ccccn4)on3)nn2)c(C)n1] | 1-(2,6-dimethylpyridin-3-yl)-N-((5-(pyridin-2- yl)isoxazol-3-yl)methyl)-1H-1,2,3-triazole-4- carboxamide |
| 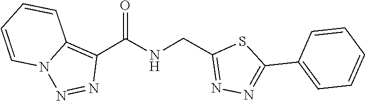 [SMILES: O=C(NCc1nnc(-c2ccccc2)s1)c1nnn2ccccc12] | N-((5-phenyl-1,3,4-thiadiazol-2-yl)methyl)- [1,2,3]triazolo[1,5-a]pyridine-3-carboxamide |
| 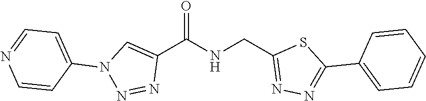 [SMILES: O=C(NCc1nnc(-c2ccccc2)s1)c1cn(-c2ccncc2)nn1] | N-((5-phenyl-1,3,4-thiadiazol-2-yl)methyl)-1- (pyridin-4-yl)-1H-1,2,3-triazole-4-carboxamide |
| 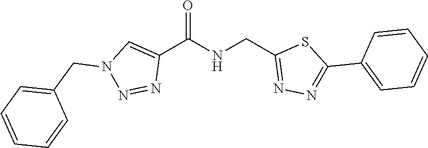 [SMILES: O=C(NCc1nnc(-c2ccccc2)s1)c1cn(Cc2ccccc2)nn1] | 1-benzyl-N-((5-phenyl-1,3,4-thiadiazol-2- yl)methyl)-1H-1,2,3-triazole-4-carboxamide |
| 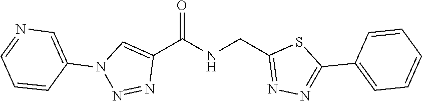 [SMILES: O=C(NCc1nnc(-c2ccccc2)s1)c1cn(-c2cccnc2)nn1] | N-((5-phenyl-1,3,4-thiadiazol-2-yl)methyl)-1- (pyridin-3-yl)-1H-1,2,3-triazole-4-carboxamide |
| 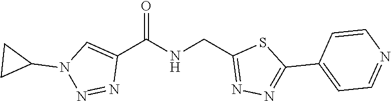 [SMILES: O=C(NCc1nnc(-c2ccncc2)s1)c1cn(C2CC2)nn1] | 1-cyclopropyl-N-((5-(pyridin-4-yl)-1,3,4- thiadiazol-2-yl)methyl)-1H-1,2,3-triazole-4- carboxamide |
| 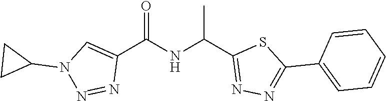 [SMILES: CC(NC(=O)c1cn(C2CC2)nn1)c1nnc(-c2ccccc2)s1] | 1-cyclopropyl-N-(1-(5-phenyl-1,3,4-thiadiazol-2- yl)ethyl)-1H-1,2,3-triazole-4-carboxamide |
| 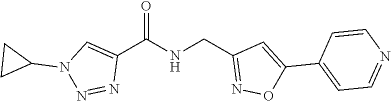 [SMILES: O=C(NCc1cc(-c2ccncc2)on1)c1cn(C2CC2)nn1] | 1-cyclopropyl-N-((5-(pyridin-4-yl)isoxazol-3- yl)methyl)-1H-1,2,3-triazole-4-carboxamide |
| 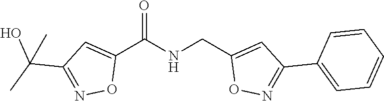 [SMILES: CC(C)(O)c1cc(C(=O)NCc2cc(-c3ccccc3)no2)on1] | 3-(2-hydroxypropan-2-yl)-N-((3-phenylisoxazol-5- yl)methyl)isoxazole-5-carboxamide |
| 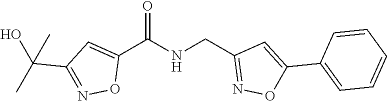 [SMILES: CC(C)(O)c1cc(C(=O)NCc2cc(-c3ccccc3)on2)on1] | 3-(2-hydroxypropan-2-yl)-N-((5-phenylisoxazol-3- yl)methyl)isoxazole-5-carboxamide |
| 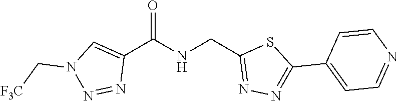 [SMILES: O=C(NCc1nnc(-c2ccncc2)s1)c1cn(CC(F)(F)F)nn1] | N-((5-(pyridin-4-yl)-1,3,4-thiadiazol-2-yl)methyl)- 1-(2,2,2-trifluoroethyl)-1H-1,2,3-triazole-4- carboxamide |
| 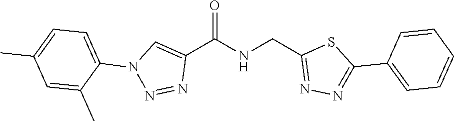 [SMILES: Cc1ccc(-n2cc(C(=O)NCc3nnc(-c4ccccc4)s3)nn2)c(C)c1] | 1-(2,4-dimethylphenyl)-N-((5-(pyridin-4-yl)-1,3,4- thiadiazol-2-yl)methyl)-1H-1,2,3-triazole-4- carboxamide |
| 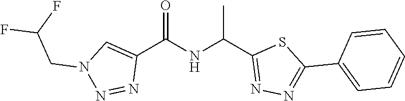 [SMILES: CC(NC(=O)c1cn(CC(F)F)nn1)c1nnc(-c2ccccc2)s1] | 1-(2,2-difluoroethyl)-N-(1-(5-phenyl-1,3,4- thiadiazol-2-yl)ethyl)-1H-1,2,3-triazole-4- carboxamide |
| 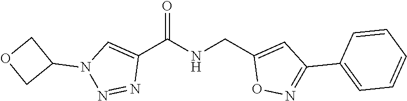 [SMILES: O=C(NCc1cc(-c2ccccc2)no1)c1cn(C2COC2)nn1] | 1-(oxetan-3-yl)-N-((3-phenylisoxazol-5-yl)methyl)- 1H-1,2,3-triazole-4-carboxamide |
| 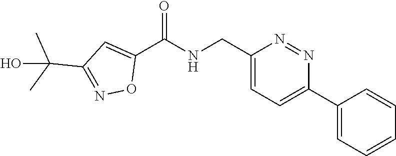 [SMILES: CC(C)(O)c1cc(C(=O)NCc2ccc(-c3ccccc3)nn2)on1] | 3-(2-hydroxypropan-2-yl)-N-((6-phenylpyridazin- 3-yl)methyl)isoxazole-5-carboxamide |
| 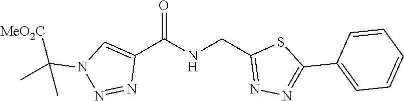 [SMILES: COC(=O)C(C)(C)n1cc(C(=O)NCc2nnc(-c3ccccc3)s2)nn1] | methyl 2-methyl-2-(4-(((5-phenyl-1,3,4-thiadiazol- 2-yl)methyl)carbamoyl)-1H-1,2,3-triazol-1- yl)propanoate |
| 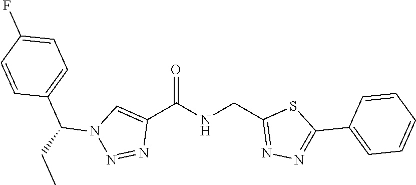 [SMILES: CC[C@H](c1ccc(F)cc1)n1cc(C(=O)NCc2nnc(-c3ccccc3)s2)nn1] | (R)-1-(1-(4-fluorophenyl)propyl)-N-((5-phenyl- 1,3,4-thiadiazol-2-yl)methyl)-1H-1,2,3-triazole-4- carboxamide |
| 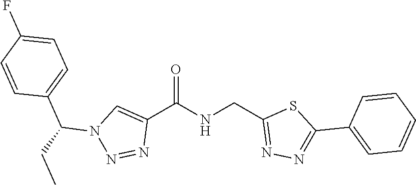 [SMILES: CC[C@H](c1ccc(F)cc1)n1cc(C(=O)NCc2nnc(-c3ccccc3)s2)nn1] | (S)-1-(1-(4-fluorophenyl)propyl)-N-((5-phenyl- 1,3,4-thiadiazol-2-yl)methyl)-1H-1,2,3-triazole-4- carboxamide |
| 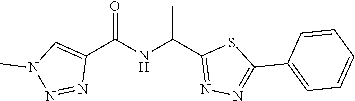 [SMILES: CC(NC(=O)c1cn(C)nn1)c1nnc(-c2ccccc2)s1] | 1-methyl-N-(1-(5-phenyl-1,3,4-thiadiazol-2- yl)ethyl)-1H-1,2,3-triazole-4-carboxamide |
| 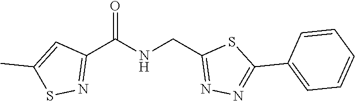 [SMILES: Cc1cc(C(=O)NCc2nnc(-c3ccccc3)s2)ns1] | 5-methyl-N-((5-phenyl-1,3,4-thiadiazol-2- yl)methyl)isothiazole-3-carboxamide |
| 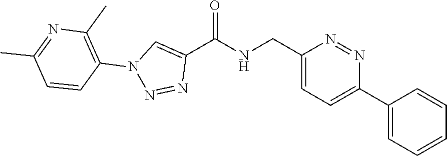 [SMILES: Cc1ccc(-n2cc(C(=O)NCc3ccc(-c4ccccc4)nn3)nn2)c(C)n1] | 1-(2,6-dimethylpyridin-3-yl)-N-((6- phenylpyridazin-3-yl)methyl)-1H-1,2,3-triazole-4- carboxamide |
| 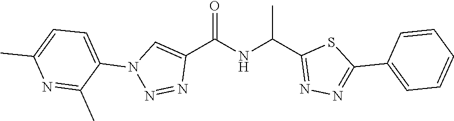 [SMILES: Cc1ccc(-n2cc(C(=O)NC(C)c3nnc(-c4ccccc4)s3)nn2)c(C)n1] | 1-(2,6-dimethylpyridin-3-yl)-N-(1-(5-phenyl-1,3,4- thiadiazol-2-yl)ethyl)-1H-1,2,3-triazole-4- carboxamide |
| 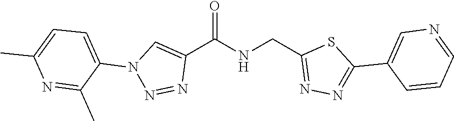 [SMILES: Cc1ccc(-n2cc(C(=O)NCc3nnc(-c4cccnc4)s3)nn2)c(C)n1] | 1-(2,6-dimethylpyridin-3-yl)-N-((5-(pyridin-3-yl)- 1,3,4-thiadiazol-2-yl)methyl)-1H-1,2,3-triazole-4- carboxamide |
| 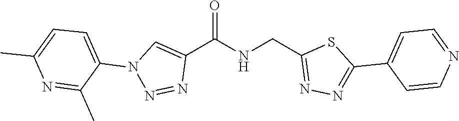 [SMILES: Cc1ccc(-n2cc(C(=O)NCc3nnc(-c4ccncc4)s3)nn2)c(C)n1] | 1-(2,6-dimethylpyridin-3-yl)-N-((5-(pyridin-4-yl)- 1,3,4-thiadiazol-2-yl)methyl)-1H-1,2,3-triazole-4- carboxamide |
| 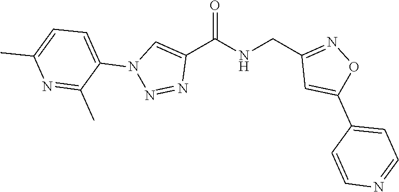 [SMILES: Cc1ccc(-n2cc(C(=O)NCc3cc(-c4ccncc4)on3)nn2)c(C)n1] | 1-(2,6-dimethylpyridin-3-yl)-N-((5-(pyridin-4- yl)isoxazol-3-yl)methyl)-1H-1,2,3-triazole-4- carboxamide |
| 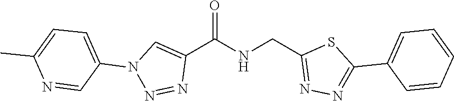 [SMILES: Cc1ccc(-n2cc(C(=O)NCc3nnc(-c4ccccc4)s3)nn2)cn1] | 1-(6-methylpyridin-3-yl)-N-((5-phenyl-1,3,4- thiadiazol-2-yl)methyl)-1H-1,2,3-triazole-4- carboxamide |
| 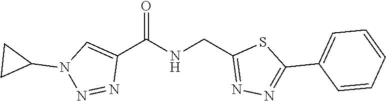 [SMILES: O=C(NCc1nnc(-c2ccccc2)s1)c1cn(C2CC2)nn1] | 1-cyclopropyl-N-((5-phenyl-1,3,4-thiadiazol-2- yl)methyl)-1H-1,2,3-triazole-4-carboxamide |
| 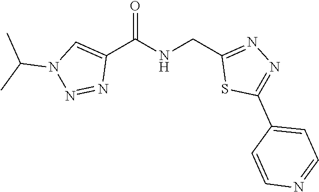 [SMILES: CC(C)n1cc(C(=O)NCc2nnc(-c3ccncc3)s2)nn1] | 1-isopropyl-N-((5-(pyridin-4-yl)-1,3,4-thiadiazol-2- yl)methyl)-1H-1,2,3-triazole-4-carboxamide |
| 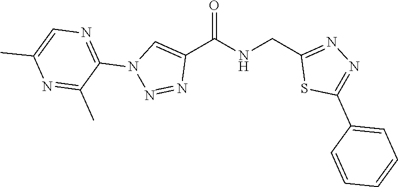 [SMILES: Cc1cnc(-n2cc(C(=O)NCc3nnc(-c4ccccc4)s3)nn2)c(C)n1] | 1-(3,5-dimethylpyrazin-2-yl)-N-((5-phenyl-1,3,4- thiadiazol-2-yl)methyl)-1H-1,2,3-triazole-4- carboxamide |
| 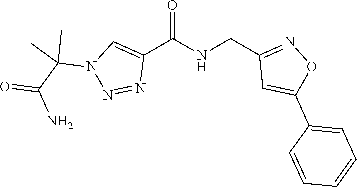 [SMILES: CC(C)(C(N)=O)n1cc(C(=O)NCc2cc(-c3ccccc3)on2)nn1] | 1-(1-amino-2-methyl-1-oxopropan-2-yl)-N-((5- phenylisoxazol-3-yl)methyl)-1H-1,2,3-triazole-4- carboxamide |
| 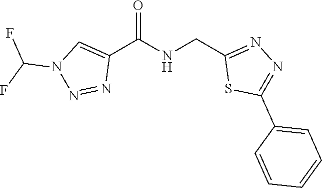 | 1-(difluoromethyl)-N-((5-phenyl-1,3,4-thiadiazol- 2-yl)methyl)-1H-1,2,3-triazole-4-carboxamide |
| 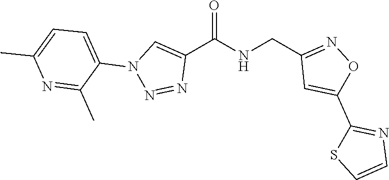 [SMILES: Cc1ccc(-n2cc(C(=O)NCc3cc(-c4nccs4)on3)nn2)c(C)n1] | 1-(2,6-dimethylpyridin-3-yl)-N-((5-(thiazol-2- yl)isoxazol-3-yl)methyl)-1H-1,2,3-triazole-4- carboxamide |
| 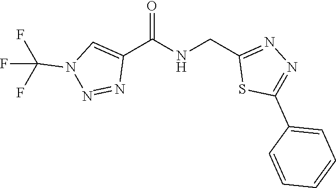 [SMILES: O=C(NCc1nnc(-c2ccccc2)s1)c1cn(C(F)(F)F)nn1] | N-((5-phenyl-1,3,4-thiadiazol-2-yl)methyl)-1- (trifluoromethyl)-1H-1,2,3-triazole-4-carboxamide |
| 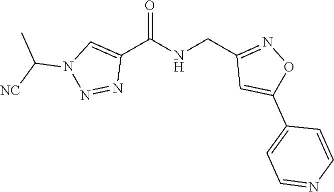 [SMILES: CC(C#N)n1cc(C(=O)NCc2cc(-c3ccncc3)on2)nn1] | 1-(1-cyanoethyl)-N-((5-(pyridin-4-yl)isoxazol-3- yl)methyl)-1H-1,2,3-triazole-4-carboxamide |
| 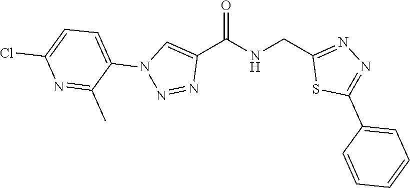 [SMILES: Cc1nc(Cl)ccc1-n1cc(C(=O)NCc2nnc(-c3ccccc3)s2)nn1] | 1-(6-chloro-2-methylpyridin-3-yl)-N-((5-phenyl- 1,3,4-thiadiazol-2-yl)methyl)-1H-1,2,3-triazole-4- carboxamide |
| 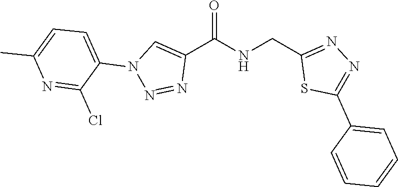 [SMILES: Cc1ccc(-n2cc(C(=O)NCc3nnc(-c4ccccc4)s3)nn2)c(Cl)n1] | 1-(2-chloro-6-methylpyridin-3-yl)-N-((5-phenyl- 1,3,4-thiadiazol-2-yl)methyl)-1H-1,2,3-triazole-4- carboxamide |
| 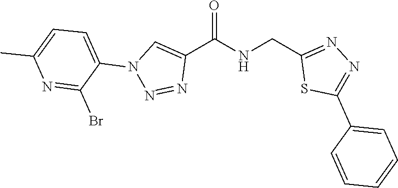 [SMILES: Cc1ccc(-n2cc(C(=O)NCc3nnc(-c4ccccc4)s3)nn2)c(Br)n1] | 1-(2-bromo-6-methylpyridin-3-yl)-N-((5-phenyl- 1,3,4-thiadiazol-2-yl)methyl)-1H-1,2,3-triazole-4- carboxamide |
| 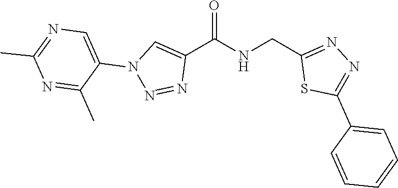 [SMILES: Cc1ncc(-n2cc(C(=O)NCc3nnc(-c4ccccc4)s3)nn2)c(C)n1] | 1-(2,4-dimethylpyrimidin-5-yl)-N-((5-phenyl-1,3,4- thiadiazol-2-yl)methyl)-1H-1,2,3-triazole-4- carboxamide |
| 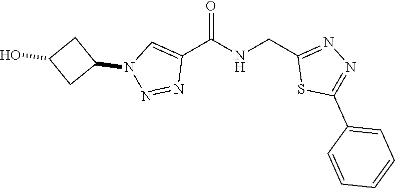 | 1-trans-3-hydroxycyclobutyl)-N-((5-phenyl-1,3,4- thiadiazol-2-yl)methyl)-1H-1,2,3-triazole-4- carboxamide |
| 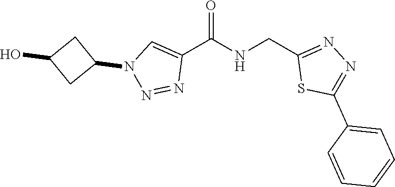 | 1-cis-3-hydroxycyclobutyl)-N-((5-phenyl-1,3,4- thiadiazol-2-yl)methyl)-1H-1,2,3-triazole-4- carboxamide |
| 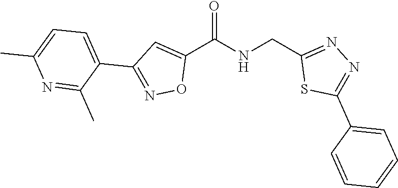 [SMILES: Cc1ccc(-c2cc(C(=O)NCc3nnc(-c4ccccc4)s3)on2)c(C)n1] | 3-(2,6-dimethylpyridin-3-yl)-N-((5-phenyl-1,3,4- thiadiazol-2-yl)methyl)isoxazole-5-carboxamide |
| 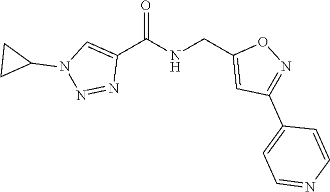 [SMILES: O=C(NCc1cc(-c2ccncc2)no1)c1cn(C2CC2)nn1] | 1-cyclopropyl-N-((3-(pyridin-4-yl)isoxazol-5- yl)methyl)-1H-1,2,3-triazole-4-carboxamide |
| 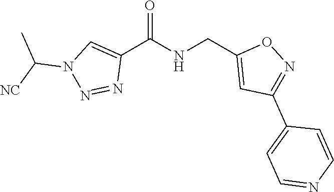 | 1-(1-cyanoethyl)-N-((3-(pyridin-4-yl)isoxazol-5- yl)methyl)-1H-1,2,3-triazole-4-carboxamide |
| 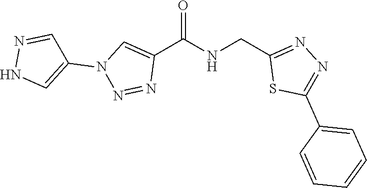 [SMILES: O=C(NCc1nnc(-c2ccccc2)s1)c1cn(-c2cn[nH]c2)nn1] | N-((5-phenyl-1,3,4-thiadiazol-2-yl)methyl)-1-(1H- pyrazol-4-yl)-1H-1,2,3-triazole-4-carboxamide |
| 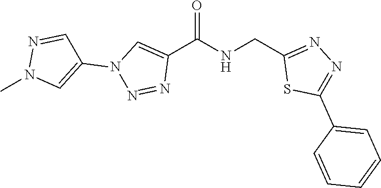 [SMILES: Cn1cc(-n2cc(C(=O)NCc3nnc(-c4ccccc4)s3)nn2)cn1] | 1-(1-methyl-1H-pyrazol-4-yl)-N-((5-phenyl-1,3,4- thiadiazol-2-yl)methyl)-1H-1,2,3-triazole-4- carboxamide |
| 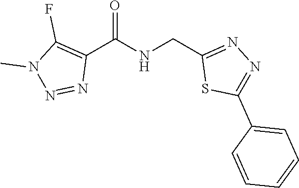 [SMILES: Cn1nnc(C(=O)NCc2nnc(-c3ccccc3)s2)c1F] | 5-fluoro-1-methyl-N-((5-phenyl-1,3,4-thiadiazol-2- yl)methyl)-1H-1,2,3-triazole-4-carboxamide |
| 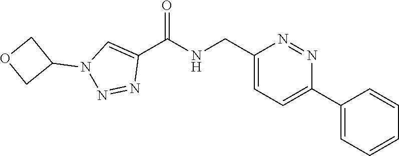 [SMILES: O=C(NCc1ccc(-c2ccccc2)nn1)c1cn(C2COC2)nn1] | 1-(oxetan-3-yl)-N-((3-phenylisoxazol-5-yl)methyl)- 1H-1,2,3-triazole-4-carboxamide |
| 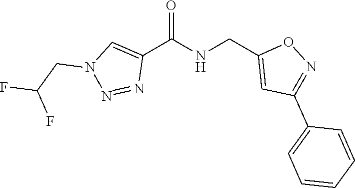 | 1-(2,2-difluoroethyl)-N-((3-phenylisoxazol-5- yl)methyl)-1H-1,2,3-triazole-4-carboxamide |
| 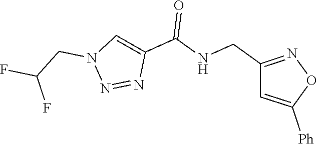 [SMILES: O=C(NCc1cc(-c2ccccc2)on1)c1cn(CC(F)F)nn1] | 1-(2,2-difluoroethyl)-N-((5-phenylisoxazol-3- yl)methyl)-1H-1,2,3-triazole-4-carboxamide |
| 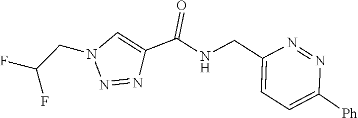 [SMILES: O=C(NCc1ccc(-c2ccccc2)nn1)c1cn(CC(F)F)nn1] | 1-(2,2-difluoroethyl)-N-((6-phenylpyridazin-3- yl)methyl)-1H-1,2,3-triazole-4-carboxamide |
| 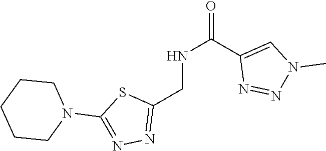 [SMILES: Cn1cc(C(=O)NCc2nnc(N3CCCCC3)s2)nn1] | 1-methyl-N-((5-(piperidin-1-yl)-1,3,4-thiadiazol-2- yl)methyl)-1H-1,2,3-triazole-4-carboxamide |
| 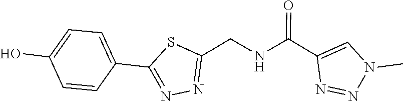 [SMILES: Cn1cc(C(=O)NCc2nnc(-c3ccc(O)cc3)s2)nn1] | N-[5-(4-hydroxyphenyl)-1,3,4-thiadiazol-2- yl]methyl]-1-methyl-triazole-4-carboxamide |
| 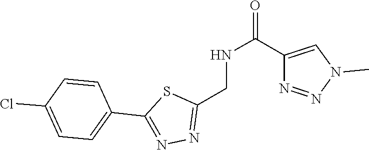 [SMILES: Cn1cc(C(=O)NCc2nnc(-c3ccc(Cl)cc3)s2)nn1] | N-((5-(4-chlorophenyl)-1,3,4-thiadiazol-2- yl)methyl)-1-methyl- 1H-1,2,3-triazole-4-carboxamide |
| 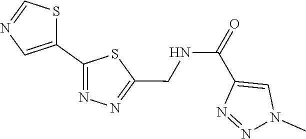 [SMILES: Cn1cc(C(=O)NCc2nnc(-c3cncs3)s2)nn1] | 1-methyl-N-((5-(thiazol-5-yl)-1,3,4-thiadiazol-2- yl)methyl)-1H-1,2,3-triazole-4-carboxamide |
| 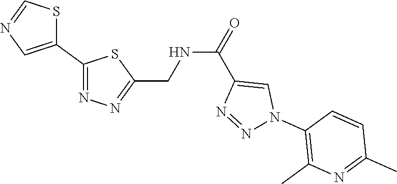 [SMILES: Cc1ccc(-n2cc(C(=O)NCc3nnc(-c4cncs4)s3)nn2)c(C)n1] | 1-(2,6-dimethylpyridin-3-yl)-N-((5-(thiazol- 5-yl)-1,3,4-thiadiazol-2-yl)methyl)-1H-1,2,3- triazole-4-carboxamide |
| 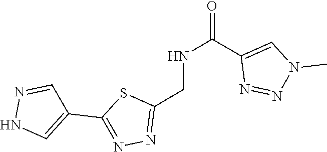 [SMILES: Cn1cc(C(=O)NCc2nnc(-c3cn[nH]c3)s2)nn1] | N-((5-(1H-pyrazol-4-yl)-1,3,4-thiadiazol-2- yl)methyl)-1-methyl-1H-1,2,3-triazole-4- carboxamide |
| 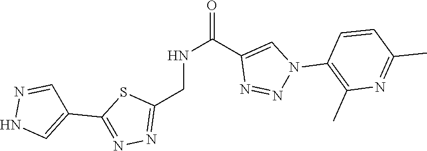 [SMILES: Cc1ccc(-n2cc(C(=O)NCc3nnc(-c4cn[nH]c4)s3)nn2)c(C)n1] | N-((5-(1H-pyrazol-4-yl)-1,3,4-thiadiazol-2- yl)methyl)-1-(2,6-dimethylpyridin-3-yl)-1H-1,2,3- triazole-4-carboxamide |
| 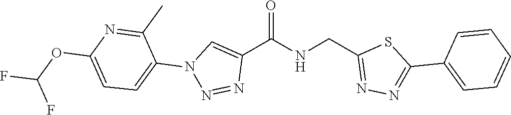 [SMILES: Cc1nc(OC(F)F)ccc1-n1cc(C(=O)NCc2nnc(-c3ccccc3)s2)nn1] | 1-(6-(difluoromethoxy)-2-methylpyridin-3-yl)-N- ((5-phenyl-1,3,4-thiadiazol-2-yl)methyl)-1H-1,2,3- triazole-4-carboxamide |
| 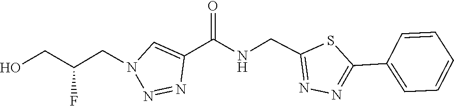 [SMILES: O=C(NCc1nnc(-c2ccccc2)s1)c1cn(C[C@H](F)CO)nn1] | (S)-1-(2-fluoro-3-hydroxypropyl)-N-((5-phenyl- 1,3,4-thiadiazol-2-yl)methyl)-1H-1,2,3-triazole-4- carboxamide |
| 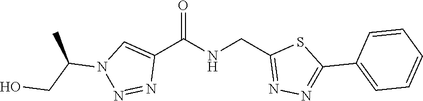 [SMILES: C[C@H](CO)n1cc(C(=O)NCc2nnc(-c3ccccc3)s2)nn1] | (R)-1-(1-hydroxypropan-2-yl)-N-((5-phenyl-1,3,4- thiadiazol-2-yl)methyl)-1H-1,2,3-triazole-4- carboxamide |
| 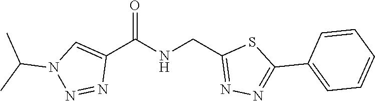 | 1-isopropyl-N-((5-phenyl-1,3,4-thiadiazol-2- yl)methyl)-1H-1,2,3-triazole-4-carboxamide |
| 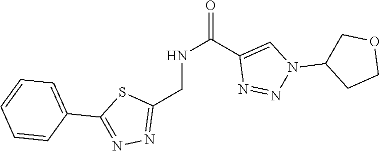 [SMILES: O=C(NCc1nnc(-c2ccccc2)s1)c1cn(C2CCOC2)nn1] | N-((5-phenyl-1,3,4-thiadiazol-2-yl)methyl)-1- (tetrahydrofuran-3-yl)-1H-1,2,3-triazole-4- carboxamide |
| 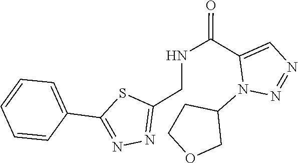 [SMILES: O=C(NCc1nnc(-c2ccccc2)s1)c1cnnn1C1CCOC1] | N-((5-phenyl-1,3,4-thiadiazol-2-yl)methyl)-1- (tetrahydrofuran-3-yl)-1H-1,2,3-triazole-5- carboxamide |
| 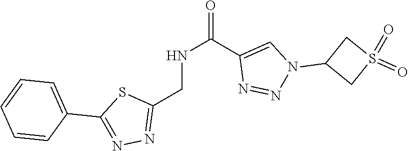 [SMILES: O=C(NCc1nnc(-c2ccccc2)s1)c1cn(C2CS(=O)(=O)C2)nn1] | 1-(1,1-dioxidothietan-3-yl)-N-((5-phenyl-1,3,4- thiadiazol-2-yl)methyl)-1H-1,2,3-triazole-4- carboxamide |
| 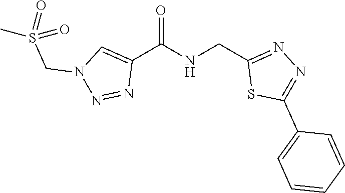 | (1-((methylsulfonyl)methyl)-N-((5-phenyl-1,3,4- thiadiazol-2-yl)methyl)-1H-1,2,3-triazole-4- carboxamide |
| 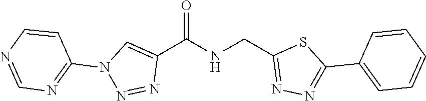 [SMILES: O=C(NCc1nnc(-c2ccccc2)s1)c1cn(-c2ccncn2)nn1] | N-((5-phenyl-1,3,4-thiadiazol-2-yl)methyl)-1- (pyrimidin-4-yl)-1H-1,2,3-triazole-4-carboxamide |
| 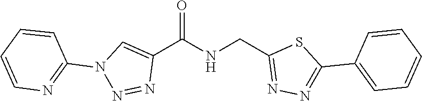 [SMILES: O=C(NCc1nnc(-c2ccccc2)s1)c1cn(-c2ccccn2)nn1] | N-((5-phenyl-1,3,4-thiadiazol-2-yl)methyl)-1- (pyridin-2-yl)-1H-1,2,3-triazole-4-carboxamide |
| 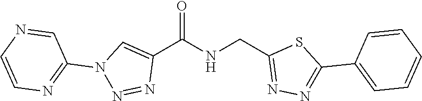 [SMILES: O=C(NCc1nnc(-c2ccccc2)s1)c1cn(-c2cnccn2)nn1] | N-((5-phenyl-1,3,4-thiadiazol-2-yl)methyl)-1- (pyrazin-2-yl)-1H-1,2,3-triazole-4-carboxamide |
| 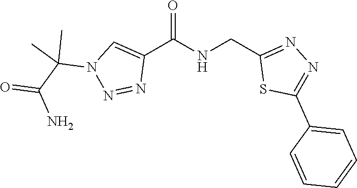 [SMILES: CC(C)(C(N)=O)n1cc(C(=O)NCc2nnc(-c3ccccc3)s2)nn1] | 1-(1-amino-2-methyl-1-oxopropan-2-yl)-N-((5- phenyl-1,3,4-thiadiazol-2-yl)methyl)-1H-1,2,3- triazole-4-carboxamide |
| 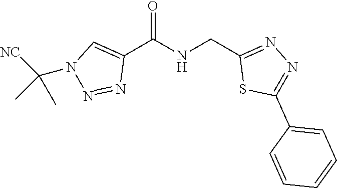 | 1-(2-cyanopropan-2-yl)-N-((5-phenyl-1,3,4- thiadiazol-2-yl)methyl)-1H-1,2,3-triazole-4- carboxamide |
| 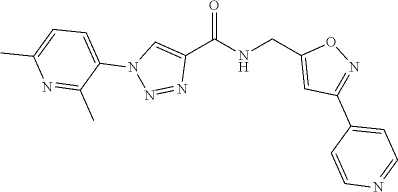 [SMILES: Cc1ccc(-n2cc(C(=O)NCc3cc(-c4ccncc4)no3)nn2)c(C)n1] | 1-(2,6-dimethylpyridin-3-yl)-N-((3-(pyridin-4- yl)isoxazol-5-yl)methyl)-1H-1,2,3-triazole-4- carboxamide |
| 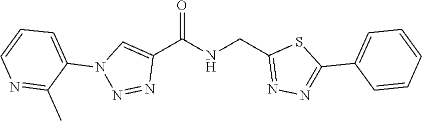 [SMILES: Cc1ncccc1-n1cc(C(=O)NCc2nnc(-c3ccccc3)s2)nn1] | 1-(2-methylpyridin-3-yl)-N-((5-phenyl-1,3,4- thiadiazol-2-yl)methyl)-1H-1,2,3-triazole-4- carboxamide |
| 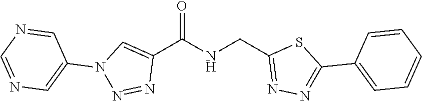 [SMILES: O=C(NCc1nnc(-c2ccccc2)s1)c1cn(-c2cncnc2)nn1] | N-((5-phenyl-1,3,4-thiadiazol-2-yl)methyl)-1- (pyrimidin-5-yl)-1H-1,2,3-triazole-4-carboxamide |
| 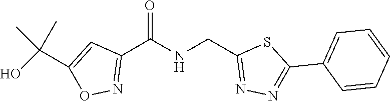 [SMILES: CC(C)(O)c1cc(C(=O)NCc2nnc(-c3ccccc3)s2)no1] | 5-(2-hydroxypropan-2-yl)-N-((5-phenyl-1,3,4- thiadiazol-2-yl)methyl)isoxazole-3-carboxamide |
| 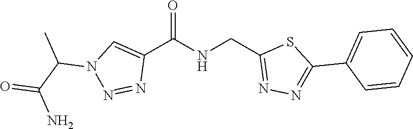 | 1-(1-amino-1-oxopropan-2-yl)-N-((5-phenyl-1,3,4- thiadiazol-2-yl)methyl)-1H-1,2,3-triazole-4- carboxamide |
| 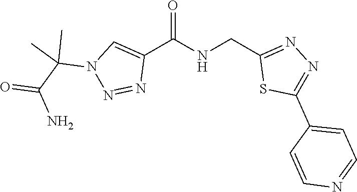 [SMILES: CC(C)(C(N)=O)n1cc(C(=O)NCc2nnc(-c3ccncc3)s2)nn1] | 1-(1-amino-2-methyl-1-oxopropan-2-yl)-N-((5- (pyridin-4-yl)-1,3,4-thiadiazol-2-yl)methyl)-1H- 1,2,3-triazole-4-carboxamide |
| 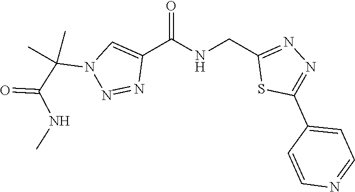 [SMILES: CNC(=O)C(C)(C)n1cc(C(=O)NCc2nnc(-c3ccncc3)s2)nn1] | 1-(2-methyl-1-(methylamino)-1-oxopropan-2-yl)- N-((5-(pyridin-4-yl)-1,3,4-thiadiazol-2-yl)methyl)- 1H-1,2,3-triazole-4-carboxamide |
| 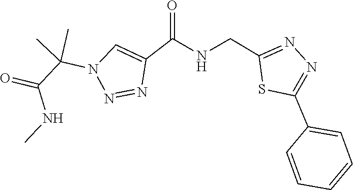 [SMILES: CNC(=O)C(C)(C)n1cc(C(=O)NCc2nnc(-c3ccccc3)s2)nn1] | 1-(2-methyl-1-(methylamino)-1-oxopropan-2-yl)- N-((5-phenyl-1,3,4-thiadiazol-2-yl)methyl)-1H- 1,2,3-triazole-4-carboxamide |
| 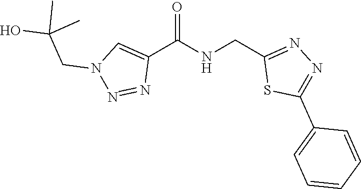 | 1-(2-hydroxy-2-methylpropyl)-N-((5-phenyl-1,3,4- thiadiazol-2-yl)methyl)-1H-1,2,3-triazole-4- carboxamide |
| 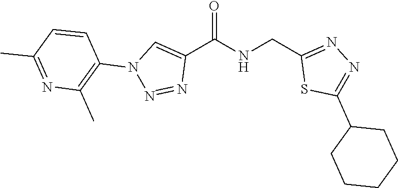 [SMILES: Cc1ccc(-n2cc(C(=O)NCc3nnc(C4CCCCC4)s3)nn2)c(C)n1] | N-((5-cyclohexyl-1,3,4-thiadiazol-2-yl)methyl)-1- (2,6-dimethylpyridin-3-yl)-1H-1,2,3-triazole-4- carboxamide |
| 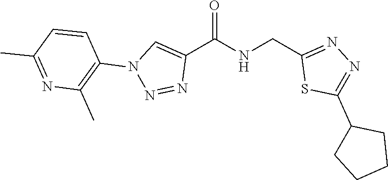 [SMILES: Cc1ccc(-n2cc(C(=O)NCc3nnc(C4CCCC4)s3)nn2)c(C)n1] | N-((5-cyclopentyl-1,3,4-thiadiazol-2-yl)methyl)-1- (2,6-dimethylpyridin-3-yl)-1H-1,2,3-triazole-4- carboxamide |
| 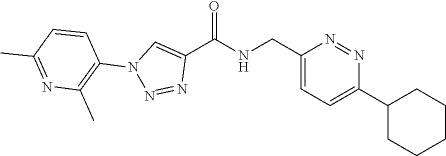 [SMILES: Cc1ccc(-n2cc(C(=O)NCc3ccc(C4CCCCC4)nn3)nn2)c(C)n1] | N-((6-cyclohexylpyridazin-3-yl)methyl)-1-(2,6- dimethylpyridin-3-yl)-1H-1,2,3-triazole-4- carboxamide |
| 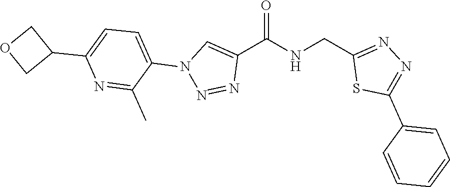 [SMILES: Cc1nc(C2COC2)ccc1-n1cc(C(=O)NCc2nnc(-c3ccccc3)s2)nn1] | 1-(2-methyl-6-(oxetan-3-yl)pyridin-3-yl)-N-((5- phenyl-1,3,4-thiadiazol-2-yl)methyl)-1H-1,2,3- triazole-4-carboxamide |
| 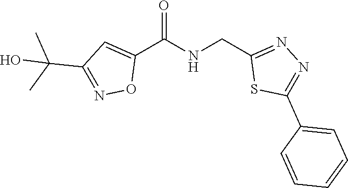 [SMILES: CC(C)(O)c1cc(C(=O)NCc2nnc(-c3ccccc3)s2)on1] | 3-(2-hydroxypropan-2-yl)-N-((5-phenyl-1,3,4- thiadiazol-2-yl)methyl)isoxazole-5-carboxamide |
| 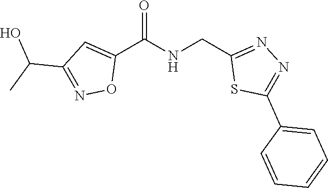 [SMILES: CC(O)c1cc(C(=O)NCc2nnc(-c3ccccc3)s2)on1] | 3-(1-hydroxyethyl)-N-((5-phenyl-1,3,4-thiadiazol- 2-yl)methyl)isoxazole-5-carboxamide |
| 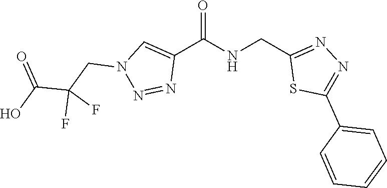 [SMILES: O=C(NCc1nnc(-c2ccccc2)s1)c1cn(CC(F)(F)C(=O)O)nn1] | 2,2-difluoro-3-(4-(((5-phenyl-1,3,4-thiadiazol-2- yl)methyl)carbamoyl)-1H-1,2,3-triazol-1- yl)propanoic acid |
| 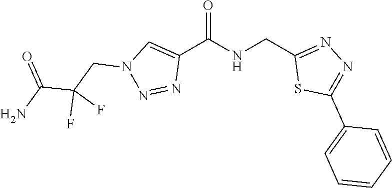 | 1-(3-amino-2,2-difluoro-3-oxopropyl)-N-((5- phenyl-1,3,4-thiadiazol-2-yl)methyl)-1H-1,2,3- triazole-4-carboxamide |
| 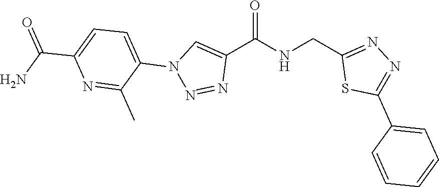 [SMILES: Cc1nc(C(N)=O)ccc1-n1cc(C(=O)NCc2nnc(-c3ccccc3)s2)nn1] | 6-methyl-5-(4-(((5-phenyl-1,3,4-thiadiazol-2- yl)methyl)carbamoyl)-1H-1,2,3-triazol-1- yl)picolinamide |
| 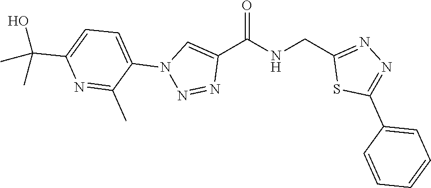 [SMILES: Cc1nc(C(C)(C)O)ccc1-n1cc(C(=O)NCc2nnc(-c3ccccc3)s2)nn1] | 1-(6-(2-hydroxypropan-2-yl)-2-methylpyridin-3- yl)-N-((5-phenyl-1,3,4-thiadiazol-2-yl)methyl)-1H- 1,2,3-triazole-4-carboxamide |
| 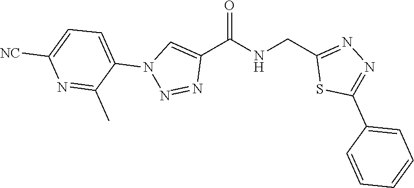 [SMILES: Cc1nc(C#N)ccc1-n1cc(C(=O)NCc2nnc(-c3ccccc3)s2)nn1] | 1-(6-cyano-2-methylpyridin-3-yl)-N-((5-phenyl- 1,3,4-thiadiazol-2-yl)methyl)-1H-1,2,3-triazole-4- carboxamide |
| 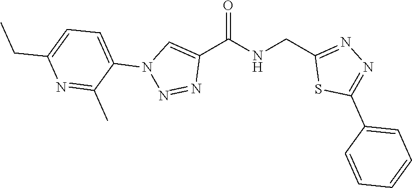 [SMILES: CCc1ccc(-n2cc(C(=O)NCc3nnc(-c4ccccc4)s3)nn2)c(C)n1] | 1-(6-ethyl-2-methylpyridin-3-yl)-N-((5-phenyl- 1,3,4-thiadiazol-2-yl)methyl)-1H-1,2,3-triazole-4- carboxamide |
| 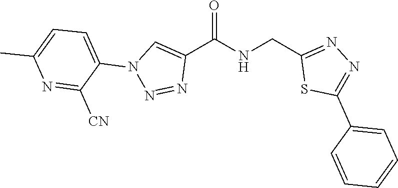 [SMILES: Cc1ccc(-n2cc(C(=O)NCc3nnc(-c4ccccc4)s3)nn2)c(C#N)n1] | 1-(2-cyano-6-methylpyridin-3-yl)-N-((5-phenyl- 1,3,4-thiadiazol-2-yl)methyl)-1H-1,2,3-triazole-4- carboxamide |
| 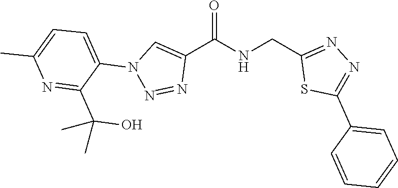 [SMILES: Cc1ccc(-n2cc(C(=O)NCc3nnc(-c4ccccc4)s3)nn2)c(C(C)(C)O)n1] | 1-(2-(2-hydroxypropan-2-yl)-6-methylpyridin-3- yl)-N-((5-phenyl-1,3,4-thiadiazol-2-yl)methyl)-1H- 1,2,3-triazole-4-carboxamide |
| 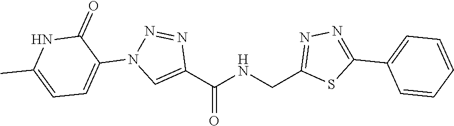 [SMILES: Cc1ccc(-n2cc(C(=O)NCc3nnc(-c4ccccc4)s3)nn2)c(=O)[nH]1] | 1-(6-methyl-2-oxo-1,2-dihydropyridin-3-yl)-N-((5- phenyl-1,3,4-thiadiazol-2-yl)methyl)-1H-1,2,3- triazole-4-carboxamide |
| 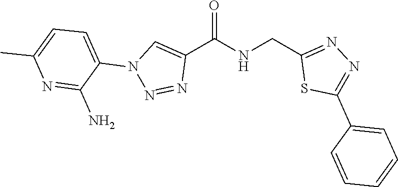 [SMILES: Cc1ccc(-n2cc(C(=O)NCc3nnc(-c4ccccc4)s3)nn2)c(N)n1] | 1-(2-amino-6-methylpyridin-3-yl)-N-((5-phenyl- 1,3,4-thiadiazol-2-yl)methyl)-1H-1,2,3-triazole-4- carboxamide |
| 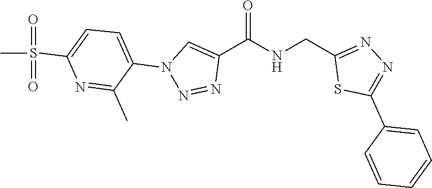 [SMILES: Cc1nc(S(C)(=O)=O)ccc1-n1cc(C(=O)NCc2nnc(-c3ccccc3)s2)nn1] | 1-(2-methyl-6-(methylsulfonyl)pyridin-3-yl)- N-((5-phenyl-1,3,4-thiadiazol-2-yl)methyl)-1H- 1,2,3-triazole-4-carboxamide |
| 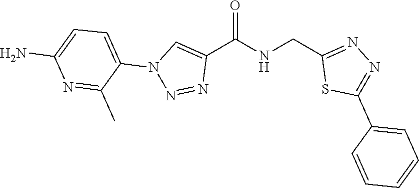 [SMILES: Cc1nc(N)ccc1-n1cc(C(=O)NCc2nnc(-c3ccccc3)s2)nn1] | 1-(6-amino-2-methylpyridin-3-yl)-N-((5-phenyl- 1,3,4-thiadiazol-2-yl)methyl)-1H-1,2,3-triazole-4- carboxamide |
| 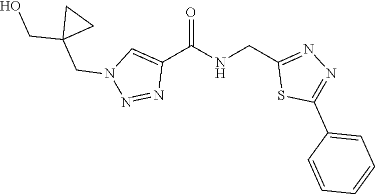 | 1-((1-(hydroxymethyl)cyclopropyl)methyl)-N-((5- phenyl-1,3,4-thiadiazol-2-yl)methyl)-1H-1,2,3- triazole-4-carboxamide |
| 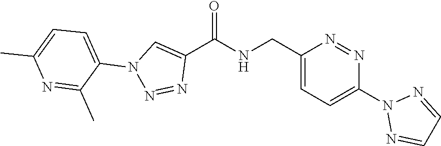 [SMILES: Cc1ccc(-n2cc(C(=O)NCc3ccc(-n4nccn4)nn3)nn2)c(C)n1] | N-((6-(2H-1,2,3-triazol-2-yl)pyridazin-3- yl)methyl)-1-(2,6-dimethylpyridin-3-yl)-1H-1,2,3- triazole-4-carboxamide |
| 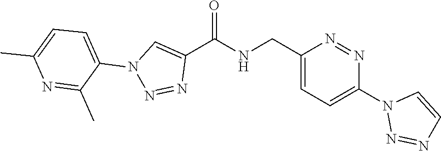 [SMILES: Cc1ccc(-n2cc(C(=O)NCc3ccc(-n4ccnn4)nn3)nn2)c(C)n1] | N-((6-(1H-1,2,3-triazol-1-yl)pyridazin-3- yl)methyl)-1-(2,6-dimethylpyridin-3-yl)-1H-1,2,3- triazole-4-carboxamide |
| 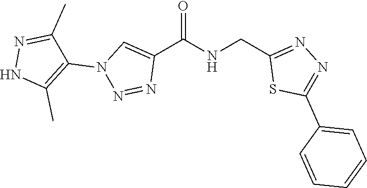 [SMILES: Cc1n[nH]c(C)c1-n1cc(C(=O)NCc2nnc(-c3ccccc3)s2)nn1] | 1-(3,5-dimethyl-1H-pyrazol-4-yl)-N-((5-phenyl- 1,3,4-thiadiazol-2-yl)methyl)-1H-1,2,3-triazole-4- carboxamide |
| 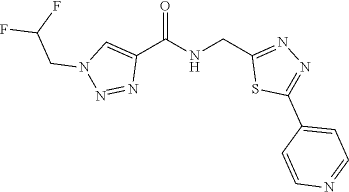 | 1-(2,2-difluoroethyl)-N-((5-(pyridin-4-yl)-1,3,4- thiadiazol-2-yl)methyl)-1H-1,2,3-triazole-4- carboxamide; and |
| 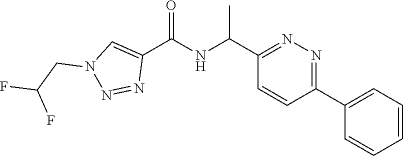 [SMILES: CC(NC(=O)c1cn(CC(F)F)nn1)c1ccc(-c2ccccc2)nn1] | 1-(2,2-difluoroethyl)-N-(1-(6-phenylpyridazin-3- yl)ethyl)-1H-1,2,3-triazole-4-carboxamide |

or a pharmaceutically acceptable salt thereof.

34. A compound having the following structure 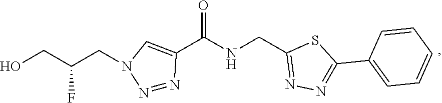 [SMILES: O=C(NCc1nnc(-c2ccccc2)s1)c1cn(C[C@H](F)CO)nn1]
or pharmaceutically acceptable salt thereof.

35. A compound having the following structure

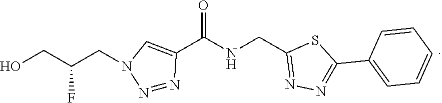 [SMILES: O=C(NCc1nnc(-c2ccccc2)s1)c1cn(C[C@H](F)CO)nn1]

36. A method of treating cancer comprising administering to a patient in need thereof a compound, or pharmaceutically acceptable salt thereof, of claim 1.

37. A method of treating cancer comprising administering to a patient in need thereof a compound, or pharmaceutically acceptable salt thereof, of claim 1 and an additional agent

38. The method of claim 37, wherein the additional agent is a PD-1 antagonist.

39. The method of claim 38, wherein the PD-1 antagonist is pembrolizumab, nivolumab, atezolizumab, durvalumab, avelumab, cemiplimab, or dostarlimab.

40. The method of claim 39, wherein the PD-1 antagonist is pembrolizumab.

41. The method of claim 36, wherein the cancer is a solid tumor or a lymphoma displaying IL4I1-expressing cells.

42. The method of claim 36, wherein the cancer is melanoma, non-small cell lung cancer, head and neck squamous cell cancer, classical Hodgkin lymphoma, primary mediastinal large B-cell lymphoma, urothelial cancer, microsatellite instability-high or mismatch repair deficient cancer, gastric cancer, esophageal cancer, cervical cancer, hepatocellular carcinoma, Merkel cell carcinoma, renal cell carcinoma, endometrial carcinoma, tumor mutational burden-high cancer, cutaneous squamous cell carcinoma, or triple negative breast cancer.

43. The method of claim 36, wherein the cancer is primary mediastinal large B-cell lymphoma, classical Hodgkin lymphoma, nodular lymphocyte predominant Hodgkin lymphoma, non-mediastinal diffuse large B-cell lymphoma, large B-cell lymphoma, acute myeloid leukemia or small lymphocytic lymphoma/chronic lymphocytic leukemia.

44. The method of treatment of claim 36, wherein the compound is administered to the patient orally.

45. A pharmaceutical composition comprising a compound of claim 1, or a pharmaceutically acceptable salt thereof and a pharmaceutically acceptable carrier.

46. A pharmaceutical composition comprising a compound of claim 1 and a pharmaceutically acceptable carrier.

47. A pharmaceutical composition comprising a compound of claim 3, or a pharmaceutically acceptable salt thereof, and a pharmaceutically acceptable carrier.
